SEQUENCE LISTING (6 sequences)

SEQ ID NO: 1 - protein, 449 aa, Artificial Sequence
  Cetuximab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Cetuximab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 223 aa, Artificial Sequence
  Matuzumab heavy chain Fab
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 4 - protein, 212 aa, Artificial Sequence
  Matuzumab light chain
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 5 - protein, 448 aa, Artificial Sequence
  Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0127913 A1
(43) Pub. Date: Apr. 24, 2025

(54) BIFUNCTIONAL FOLATE RECEPTOR BINDING COMPOUNDS

(71) Applicant: LYCIA THERAPEUTICS, INC., South San Francisco, CA (US)

(72) Inventors: Jason G. LEWIS, South San Francisco, CA (US); Eric D. TURTLE, South San Francisco, CA (US); Tao CHEN, South San Francisco, CA (US); Darin HILDEBRANDT, South San Francisco, CA (US)

(21) Appl. No.: 18/568,779

(22) PCT Filed: Jun. 24, 2022
(86) PCT No.: PCT/US2022/073164

§ 371 Date: Dec. 8, 2023

Related U.S. Application Data
(60) Provisional application No. 63/214,773, filed on Jun. 24, 2021.
(60) Provisional application No. 63/214,774, filed on Jun. 24, 2021.

(51) Int. Cl. A61K 47/68; A61K 47/55

(52) U.S. Cl. CPC A61K 47/6803; A61K 47/55

(57) ABSTRACT

The present disclosure provides a class of compounds including a ligand moiety that specifically binds to a cell surface receptor, such as a folate receptor. The cell surface folate binding compound can trigger the receptor to internalize into the cell a bound compound. The ligand moieties of this disclosure can be linked to a variety of moieties of interest without impacting the specific binding to, and function of, the cell surface receptor, e.g., folate receptor. Also provided are compounds that are conjugates of the ligand moieties linked to a biomolecule, such as an antibody, which conjugates can harness cellular pathways to remove specific proteins of interest from the cell surface or from the extracellular milieu. Also provided herein are methods of using the conjugates to target a polypeptide of interest for sequestration and/or lysosomal degradation.

### 1. CROSS REFERENCE TO RELATED APPLICATIONS

This application claims the benefit of U.S. Provisional Application No. 63/214,774, filed Jun. 24, 2021, and U.S. Provisional Application No. 63/214,773 filed Jun. 24, 2021, which are hereby incorporated in their entirety by reference.

### 2. INTRODUCTION

Folate receptors on cells can bind ligands such as folate and reduced folic acid derivatives to mediate delivery of molecules containing such ligands into the interior of the cells. Human proteins from the family include folate receptor 1, folate receptor 2, and folate receptor gamma. Folate-based diagnostic and therapeutic agents are used intracellularly.

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches. Thus, there is a need for therapies that can target a wider range of proteins.

Thus, there is a need for therapies that can target a wider range of proteins. For example, there is a need for therapies that can harness a subject's own cellular pathways to remove specific proteins of interest from the cell surface or the extracellular milieu.

### 3. SUMMARY OF THE INVENTION

The present disclosure provides a class of compounds including a ligand moiety that specifically binds to a cell surface receptor such as a folate receptor. The cell surface folate binding compound can trigger the receptor to internalize into the cell a bound compound. The ligand moieties of this disclosure can be linked to a variety of moieties of interest without impacting the specific binding to, and function of, the cell surface folate receptor, and provide for internalization of the linked moieties of interest into the cell. In some embodiments, ligand moieties of this disclosure can be linked to a variety of TNFα inhibitor moieties without impacting the specific binding to, and function of, the cell surface receptor, e.g., folate receptor. For example, the bifunctional compounds described herein linked to a TNFα inhibitor may sequester and/or degrade TNFα in a cell's lysosome. In some embodiments, the linked moiety of interest is itself targeted for delivery or internalization in the cell. Also provided are compounds that are conjugates of the ligand moieties linked to a biomolecule, such as an antibody, which conjugates can harness cellular pathways to remove specific proteins of interest from the cell surface or from the extracellular milieu. For example, the conjugates described herein may sequester and/or degrade a target molecule of interest in a cell's lysosome. Also provided herein are compositions comprising such bifunctional molecules and conjugates and methods of using the bifunctional molecules and conjugates to target a polypeptide of interest for sequestration and/or lysosomal degradation, and methods of using the bifunctional compounds and conjugates to treat disorders or disease.

A first aspect of this disclosure includes a cell surface folate receptor binding compound of formula (I):

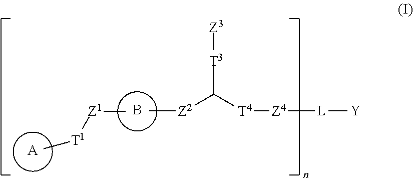

or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted urea, optionally substituted sulfonamide, optionally substituted thiourea, —NR²¹—, —O—, —S—, and optionally substituted (C₁-C₆)alkylene;
Z³ is absent, carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);
each R²¹ is independently selected from H, and optionally substituted (C₁-C₆)alkyl; n is 1 to 100;
L is a linker;
Y is a moiety of interest; and
A is a ring system of formula (II):

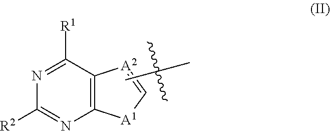

or a tautomer thereof,
wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl;
with the proviso that at least one of following applies:
1) T³ is optionally substituted (C₁-C₆)alkylene (e.g., —CH₂CH₂—);
2) L is a non-cleavable linker and Y is an extracellular target-binding moiety;
3) when A is of formula (II-A) or (II-A′), or a tautomer thereof:

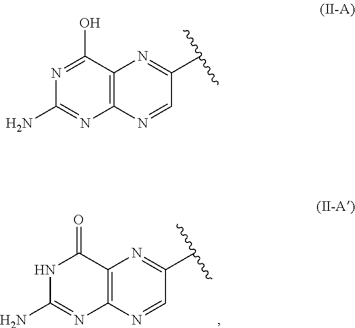 [SMILES: *c1cnc2nc(N)nc(O)c2n1 ; *c1cnc2nc(N)[nH]c(=O)c2n1]

then Z¹ is not NR²¹, and/or B is not 1,4-linked phenyl;
4) when A is of formula (II-B), or a tautomer thereof:

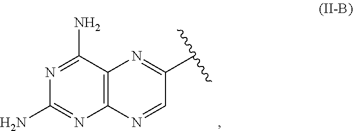 [SMILES: *c1cnc2nc(N)nc(N)c2n1]

then Z¹ is not NR²¹, and/or B is not 1,4-linked phenyl; and/or
5) when A is of formula (II-C) or (II-C′), or a tautomer thereof:

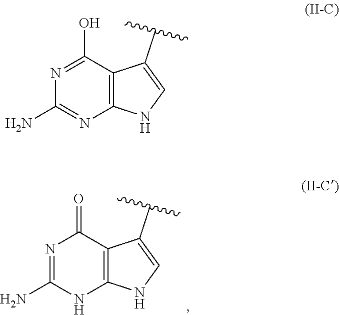 [SMILES: *c1c[nH]c2nc(N)nc(O)c12 ; *c1c[nH]c2[nH]c(N)nc(=O)c12]

then T¹-Z¹ is not —CH₂CH₂—, and/or B is not phenyl.

In some embodiments of formula (I), Y is antibody or antibody fragment that specifically binds the target protein and the compound is of formula (VIIIa):

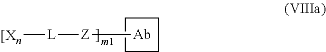

or a pharmaceutically acceptable salt thereof,
wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

In some embodiments, Y is a TNFα inhibitor. In some embodiments, provided herein are TNFα-degrading compounds of formula (I-A):

X-L-Y (I-A)

or a salt thereof, wherein:
X is a moiety that binds a cell surface folate receptor;
L is a linker having a backbone of 10 to 50 atoms in length; and
Y is an allosteric desymmetrization TNFα inhibitor.

A second aspect of this disclosure includes a method of internalizing a target protein in a cell comprising a cell surface receptor selected from a folate receptor, where the method includes contacting a cellular sample comprising the cell and the target protein with an effective amount of a compound (e.g., as described herein) that specifically binds the target protein and specifically binds the cell surface receptor to facilitate cellular uptake of the target protein.

A third aspect of this disclosure includes a method of reducing levels of a target protein in a biological system, where the method includes contacting the biological system with an effective amount of a compound (e.g., as described herein) that specifically binds the target protein and specifically binds a cell surface receptor of cells in the biological system to facilitate cellular uptake and degradation of the target protein.

A fourth aspect of this disclosure includes a method of treating a disease or disorder associated with a target protein, where the method includes administering to a subject in need thereof an effective amount of a compound (e.g., as described herein), wherein the compound specifically binds the target protein.

In certain embodiments the disease or disorder is an inflammatory disease, an autoimmune disease, or a cancer.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows surface plasmon resonance (SPR) sensorgrams measuring 1:1 binding of compound (1-21) to folate receptor 2 (FOLR2). Further details are described in Example 51 of the experimental section.

FIGS. 2A-2C show SPR sensorgrams which illustrate binding kinetics of exemplary compounds to TNFα trimer. SPR binding of compound (1-16) to TNFα trimer (FIG. 2A), compound (1-21) to TNFα trimer (FIG. 2B), and compound (1-25) to TNFα trimer (FIG. 2C). Further details are described in Example 52 of the experimental section.

FIGS. 3A-3B show SPR sensorgrams which demonstrate co-engagement of exemplary compound (1-21) TNFα trimer complex to folate receptor (FIG. 3A), and exemplary compound (1-18) TNFα trimer complex to folate receptor (FIG. 3B). Further details are described in Example 53 of the experimental section.

FIG. 4 shows that exemplary compound (1-17) stimulated uptake of labelled TNFα in THP-1 cells in a dose-dependent manner as measured by median fluorescence intensity of pHrodo dye. Further details are described in Example 54 of the experimental section.

FIG. 5 shows that exemplary compound (I-17) mediated degradation and rescue of TNFα in THP-1 cells. Further details are described in Example 55 of the experimental section.

FIG. 6 shows that exemplary compound (1-17) mediated depletion of TNFα from the media of THP-1 cells. Further details are described in Example 56 of the experimental section.

FIGS. 7A-7B demonstrate folate receptor-dependent uptake of target protein IgE using an exemplary omalizumab-folate receptor ligand conjugate of this disclosure. FIG. 7A shows cell uptake of IgE-Alexa647 was enhanced across the dose range in both the wild type (WT) and folate receptor 2 (FOLR2) over expressing cells with the exemplary omalizumab-folate receptor ligand conjugate. FIG. 7B shows increased uptake was observed using the exemplary conjugate in FOLR2 overexpressing THP-1 cells compared to WT cells, and addition of folic acid decreased that uptake back to the WT level. Further details are described in Example 57 of the experimental section.

FIGS. 8A-8B illustrates the stimulation of uptake and degradation of target protein DQ-BSA by an exemplary conjugate. FIG. 8A shows the exemplary conjugate enhanced uptake of DQ-BSA and resulted in proteolysis and dequenching of BODIPY dye in the endolysosomal pathway. In the presence of protease inhibitors (PI), the intracellular fluorescent signal was diminished. FIG. 8B shows that the uptake and degradation is folate receptor mediated. In the presence of folic acid (FA) the fluorescent signal of anti-BSA control antibody without folate (anti-BSA) was the same as anti-BSA conjugate with the folate receptor ligand (anti-BSA/Compound I-4B conjugate).

FIG. 9 illustrates complex formation observed on SPR for example compounds (TNFα+10 nM compound binding FOLR2), where the example compounds are: I-14, I-15, I-22, I-23, I-24, I-16, I-18, I-20, I-21, I-25, and I-26.

FIG. 10A-10C illustrates example compound mediated uptake of TNFα in THP-1 cells. Illustrated are comparisons of compounds with various linker lengths (FIG. 10A), and comparison of compounds with various TNFα binding moieties (“Y”) (FIGS. 10B, and 10C). Note in FIG. 10A-10C: “N” represents the number of ethylene glycol moieties in the linker.

FIG. 11 illustrates affinity-dependent clearance of an OMA-folate example compound (OMA-I-4B) as compared to OMA by itself and an OMA-ref. compound with reduced affinity.

FIG. 12 illustrates example compound mediated uptake of TNFα in THP-1 cells. As shown in FIG. 12 compounds I-87 and I-92 both showed a dose-dependent uptake of TNFα compared the no compound control (DMSO) with compound I-87 showing greater potency (EC50=15.29 nM).

### 5. DETAILED DESCRIPTION OF THE INVENTION

As summarized above, this disclosure provides classes of compounds including a ligand moiety that specifically binds to a cell surface receptor. Also provided herein are conjugates that comprise a moiety, X, that binds to such a cell surface receptor, for example, an internalizing cell surface receptor, for example, for sequestration and/or lysosomal degradation. In certain embodiments, the cell surface receptor is a folate receptor. In some embodiments this disclosure provides lysosome-targeting bifunctional compounds that specifically bind TNFα (also referred to as TNF-alpha). For example, provided herein are bifunctional compounds including a binding moiety for a cell surface receptor (e.g., a folate receptor), covalently connected via a linker to a TNFα binding moiety.

This disclosure includes compounds of formulae (I), (IIIA) and (IIIB) (e.g., as described in more detail herein below).

Each component of the bifunctional compounds of this disclosure, conjugates and methods of this disclosure are described in greater detail below. A particular class of folate binding compounds is described. Also described are biomolecule conjugates that include a cell surface receptor binding moiety (X) that binds to a folate receptor. Linkers (L) and moieties of interest (Y) which find use in the folate binding compounds, and the biomolecule conjugates are also described. Methods in which the compounds and conjugates of this disclosure find use are also described.

### 5.1. Folate Receptor Binding Compounds

As summarized above, this disclosure provides a class of compounds including a ligand moiety that specifically binds to a cell surface folate receptor. The folate receptor ligand moieties of this disclosure can be linked to a variety of moieties of interest without impacting the specific binding to, and function of, the cell surface folate receptor. The inventors have demonstrated that compounds of this disclosure can utilize the functions of cell surface folate receptors in a biological system, e.g., for internalization and sequestration of a compound to the lysosome of a cell, and in some cases subsequent lysosomal degradation. The compounds of this disclosure find use in a variety of applications.

The compounds of this disclosure can specifically bind to a cell surface folate receptor, for example, an internalizing folate cell surface receptor. In particular embodiments, the surface folate receptor is a human folate receptor. In particular embodiments, the folate receptor is folate receptor 1 (FRα). In certain cases, the folate receptor is folate receptor 2 (FRβ).

The folate binding compounds of this disclosure include a moiety (X) that specifically binds to the cell surface folate receptor. The folate binding compounds can be monovalent or multivalent (e.g., bivalent or trivalent or of higher valency), where a monovalent compound includes a single folate receptor ligand moiety, and a monovalent compound includes two or more such moieties.

A compound comprising such X (e.g., as described herein), may bind to other receptors, for example, may bind with lower affinity as determined by, e.g., immunoassays or other assays known in the art. In a specific embodiment, X, or a compound as described herein comprising such X specifically binds to the cell surface folate receptor with an affinity that is at least 2 logs, 2.5 logs, 3 logs, 4 logs or greater than the affinity when X or the compound or the conjugate bind to another cell surface receptor. In a specific embodiment, X, or a compound as described herein comprising X, specifically binds to a folate receptor with an affinity (K_{d}) less than or equal to 20 mM. In particular embodiments, such binding is with an affinity (K_{d}) less than or equal to about 20 mM, about 10 mM, about 1 mM, about 100 uM, about 10 uM, about 1 uM, about 100 nM, about 10 nM, or less than or equal to about 1 nM. Unless otherwise noted, “binds,” “binds to,” “specifically binds” or “specifically binds to” in this context are used interchangeably.

In certain embodiments, the folate receptor binding moiety X is able to bind to a folate specific cell surface receptor, and direct (or target) the molecule to this receptor. In certain embodiments, the folate receptor binding moiety X is capable of binding to the folate receptor and directing (or targeting) a compound or conjugate described herein for internalization and sequestration to the lysosome, and/or subsequent lysosomal degradation.

In some embodiments, the folate binding moiety X includes a folate heterocyclic ring, or analog thereof, that is linked via a linking moiety comprising a cyclic group (e.g., aryl, heteroaryl, heterocycle, or cycloalkyl) to an amino acid derivative (e.g., a glutamic acid). The linking moiety can be of 1-10 atoms in length, such as 1-6, or 1-5 atoms in length. The linking moieties cyclic group can be any convenient group including, aryl, (e.g., phenyl), heteroaryl, (e.g., pyridine, thiophene), heterocyclic (e.g., piperidine), cycloalkyl (e.g., cyclohexyl), and bicycloalkyl groups. In some embodiments, the linking moieties cyclic group is aryl. The amino acid derivative can be any convenient amino acid group including, glutamic acid, and aspartic acid.

In some embodiments, the folate heterocyclic ring of X is linked via an optionally substituted aryl or heteroaryl group to an amino acid derivative (e.g., a glutamic acid) that together provide a moiety having a desirable binding affinity and activity at the folate receptor of interest. Multiple folate binding moieties X can be linked together to provide multivalent binding to the folate receptor. The folate binding moiety or moieties X can be further linked to any convenient moiety or molecule of interest (e.g., as described herein). In certain embodiments, the folate binding moiety X includes a glutamic acid moiety that is linked to a molecule of interest via a linker. In certain cases, the folate binding moiety X is linked to the molecule of interest via a linker covalently bonded to the gamma position of the glutamic acid moiety. In other cases, the folate binding moiety X is linked to the molecule of interest via a linker covalently bonded to the alpha position of the glutamic acid moiety.

Accordingly, provided herein are folate binding moiety X, of formula (Ia):

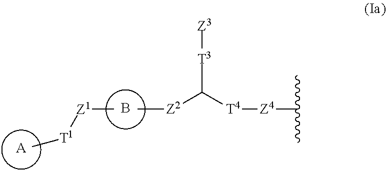

wherein:
A is a ring system of formula (XII):

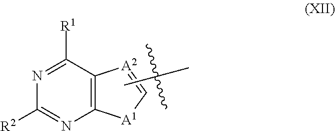

or a tautomer thereof, wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is absent, carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, sulfonamide, amine, ether, optionally substituted aryl, optionally substituted heterocycle, and optionally substituted heteroaryl);
each R²¹ is independently selected from H, and optionally substituted (C₁-C₆)alkyl; and [symbol] represents the point of attachment to -L-Y (e.g., as described herein).

The folate binding moiety X of formula (Ia) can be incorporated into the compounds of this disclosure by attachment of a moiety of interest (Y) to the Z⁴ group via a linking moiety. It is understood that in the compounds of formula (Ia), the group or linking moiety attached to Z⁴ can, in some cases, be considered to be part of the folate binding moiety (X) and provide for desirable binding to the folate receptor. In certain other cases, the group or linking moiety attached to Z⁴ can be considered part of the linker L (e.g., of formula (IV) as described herein).

Accordingly, in one embodiment, provided herein are folate binding compounds of formula (I):

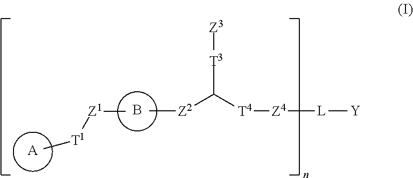

or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is absent, carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);
each R²¹ is independently selected from H, and optionally substituted (C₁-C₆)alkyl;
n is 1 to 100;
L is a linker;
Y is a moiety of interest; and
A is a ring system of formula (II):

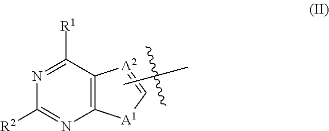

or a tautomer thereof, wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl.

In some embodiments of formula (I), at least one of following applies:
1) T³ is optionally substituted (C₁-C₆)alkylene (e.g., —CH₂CH₂—);
2) L is a non-cleavable linker and Y is an extracellular target-binding moiety;
3) when A is of formula (II-A) or (II-A′), or a tautomer thereof:

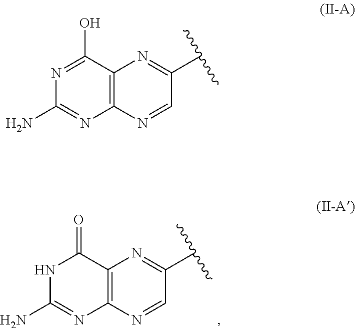 [SMILES: *c1cnc2nc(N)nc(O)c2n1 ; *c1cnc2nc(N)[nH]c(=O)c2n1]

then Z¹ is not NR²¹, and/or B is not 1,4-linked phenyl;
4) when A is of formula (II-B), or a tautomer thereof:

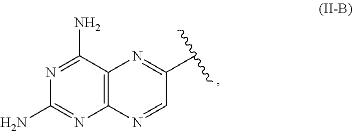 [SMILES: *c1cnc2nc(N)nc(N)c2n1]

then Z¹ is not NR²¹, and/or B is not 1,4-linked phenyl; and/or
5) when A is of formula (II-C) or (II-C′), or a tautomer thereof:

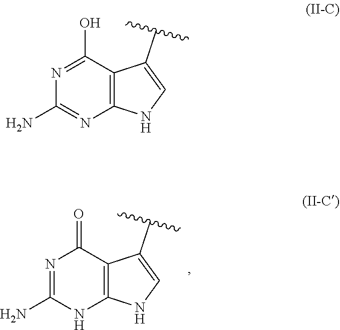 [SMILES: *c1c[nH]c2nc(N)nc(O)c12 ; *c1c[nH]c2[nH]c(N)nc(=O)c12]

then T¹-Z¹ is not —CH₂CH₂—, and/or B is not phenyl.

In some embodiments of formula (I), T³ is optionally substituted (C₁-C₆)alkylene. In certain cases, T³ is (C₁-C₆)alkylene, i.e., hexyl, pentyl, butyl, propyl, ethyl or methyl. In certain cases, T³ is (C₁-C₃)alkylene. In certain cases, T³ is-CH₂CH₂CH₂—. In certain cases, T³ is —CH₂CH₂—. In certain cases, T³ is —CH₂—.

In some embodiments of formula (I), T⁴ is absent. Accordingly, in some embodiments, the compound is of formula (IIIA):

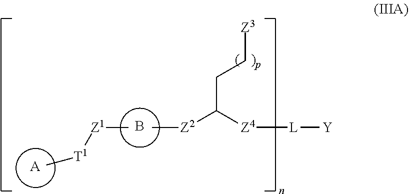

wherein p is 0 or 1.

In certain other embodiments of formula (I), T⁴ is optionally substituted (C₁-C₆)alkylene. In certain cases, each T⁴ is (C₁-C₆)alkylene, i.e., hexyl, pentyl, butyl, propyl, ethyl or methyl. In certain cases, each T⁴ is (C₁-C₃)alkylene. In certain cases, each T⁴ is-CH₂CH₂CH₂—. In certain cases, each T⁴ is —CH₂CH₂—. In certain cases, each T⁴ is —CH₂—.

In some embodiments of formula (I), T³ is absent. Accordingly, in some embodiments, the compound is of formula (IIIB):

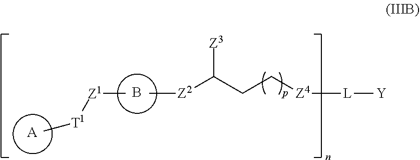

wherein p is 0 or 1.

In certain embodiments of any one of formulae (I), (IIIA) or (IIIB), Z³ is a carboxyl group, or a prodrug thereof. In certain other embodiments, Z³ is a carboxyl bioisostere, or a prodrug thereof. A carboxyl bioisostere is a group with similar physical or chemical properties to a carboxyl group. In certain cases, the carboxyl bioisostere produces broadly similar biological properties to the corresponding carboxyl group. In certain cases, the carboxyl bioisostere may modify the activity of the compound, and may alter the metabolism of the compound. The subject compounds can include both acyclic and cyclic carboxylic acid bioisosteres. Carboxyl bioisosteres that can be utilized in the subject compounds includes, but is not limited to, hydroxamic acids, phosphonic acids, sulphonic acids, sulfonamides, acylsulfonamides, sulfonylureas, tetrazoles, thiazolidinediones, oxazolidinediones, 5-oxo-1,2,4-oxadiazole, 5-oxo-1,2,4-thiadiazole, 5-thioxo-1,2,4-oxadiazole, isothiazoles, difluorophenols, tetramic acids, squaric acids, 3-hydroxyquinolin-2-ones, and 4-hydroxyquinolin-2-ones. In certain embodiments, the carboxyl bioisostere is a moiety as described in Ballatore et al. 2013, ChemMedChem., 8(3): 385-395.

In certain embodiments, a prodrug derivative of the carboxyl bioisostere group (Z³) may be incorporated into the compounds. For example, an ester prodrug group (e.g., —CO₂Et, or —CO₂CH₂CH₂—R″, where R″ is a heterocycle, e.g., N-morpholino) is included instead of a carboxylic acid group. Exemplary ester containing compounds are described herein. See e.g., compounds 1-56 and 1-57 of Table 6.

The term “pro-drug” refers to an agent which is converted into the drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drag. An example, without limitation, of a pro-drug would be a compound of the present disclosure wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

In certain embodiments, the carboxyl bioisostere, or a prodrug thereof, is a moiety of one of the following structures:

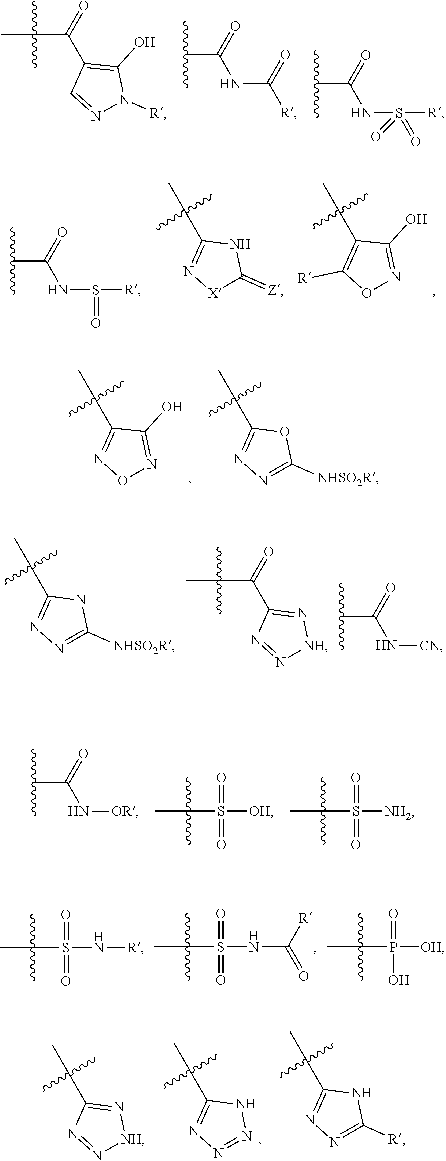

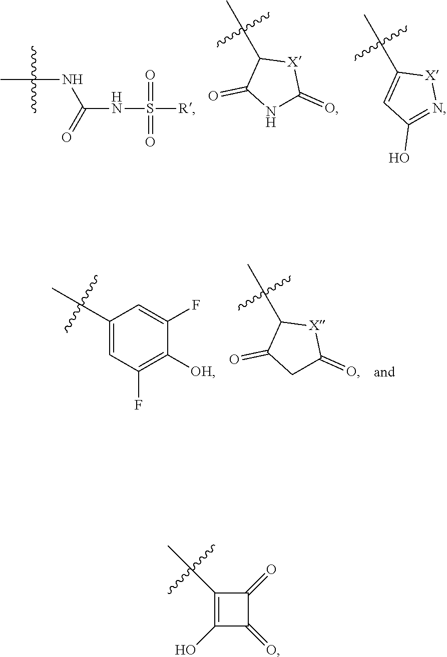

where:
each R′ is independently H or an optionally substituted moiety selected from (C₁₋₁₀)alkyl, (C₂₋₁₀)alkenyl, (C₂₋₁₀)heteroalkyl, (C₃₋₈)cyclic ring selected from cycloalkyl, aryl, heterocycle, or heteroaryl;
each X′ is independently O or S; and

### X″ is NH, O, or CH₂.

In certain embodiments, Z³ is selected from —COOH, —COOR²², —CH₂OH, —CH₂OR²², —CN, and tetrazole, wherein R²² is optionally substituted (C₁-C₆)alkyl. In certain cases, Z³ is —COOH. In certain cases, Z³ is —COOR²², and R²² is optionally substituted (C₁₋₃)alkyl. In certain cases, R²² is methyl, ethyl or propyl. In certain cases, R²² is substituted methyl, ethyl, or propyl. In certain cases, Z³ is —CH₂OH, or —CH₂OR²², and R²² is optionally substituted (C₁₋₃)alkyl. In certain cases, Z³ is —CN. In certain cases, Z³ is tetrazole.

In certain embodiments, Z³ is selected from one of the following structures:

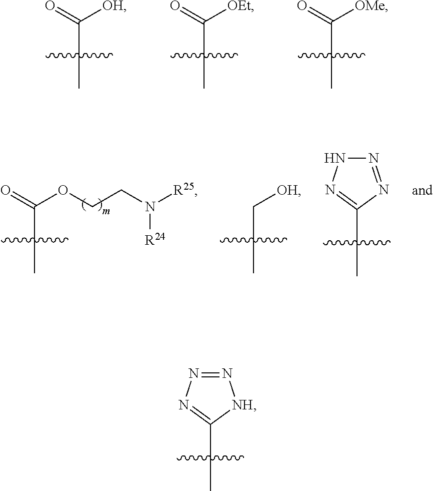

wherein:
R²⁴ and R²⁵ are independently selected from H and optionally substituted (C₁-C₆)alkyl, or R²⁴ and R²⁵ are cyclically linked to provide an optionally substituted 5 or 6-membered heterocycle; and
m is 1 to 5. In certain cases, R²⁴ and R²⁵ are H. In certain embodiments, R²⁴ and R²⁵ is optionally substituted (C₁₋₃)alkyl. In certain cases, R²⁴ and R²⁵ are cyclically linked to provide an optionally substituted 5-membered heterocycle. In certain other cases, R²⁴ and R²⁵ are cyclically linked to provide an optionally substituted 6-membered heterocycle. In certain cases, Z³ is of the following structure:

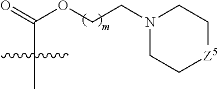

wherein
Z⁵ is O, NH or NR²¹; and
R²¹ is (C₁-C₆)alkyl.

In certain cases, Z⁵ is O and m is 1. In certain cases, Z⁵ is NH, and m is 1. In certain cases, Z⁵ is NCH₃, and m is 1.

In certain embodiments of any one of formulae (I), (IIIA) or (IIIB), Z³ is absent. Accordingly, in some embodiments, the compound is of formula (IIIC):

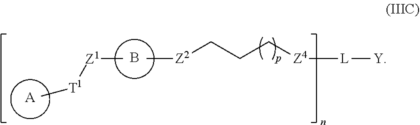

In some embodiments of any one of formulae (I), (IIIA), (IIIB, or (IIIC), Z² is a linking moiety (e.g., as described herein). In certain cases, Z² is an optionally substituted amide. In certain cases, Z² is an optionally substituted sulfonamide. In certain cases, Z² is an optionally substituted urea. In certain cases, Z² is an optionally substituted thiourea. In certain embodiments, Z² is —CONR²¹—. In certain cases, Z² is —O—. In certain case, Z² is —S—. In certain cases, Z² is an optionally substituted (C₁-C₆)alkylene. In certain cases, Z² is an amide bioisostere (e.g., as described herein below).

In certain embodiments, Z² is —CONR²¹—, wherein R²¹ is selected from H, and optionally substituted (C₁-C₆)alkyl. In certain cases, R²¹ is H. In certain other cases, R²¹ is optionally substituted (C₁-C₃)alkyl. In certain cases, R²¹ is methyl. In certain cases, R²¹ is ethyl.

In certain embodiments, Z² is —CONR²¹—, —SO₂NR²¹—, —NR²¹CO—, —NR²¹C(═O)NR²¹—, or —NR²¹C(═S)NR²¹, wherein each R²¹ is independently selected from H, and optionally substituted (C₁-C₆)alkyl. In certain cases, each R²¹ is H. In certain other cases, each R² is optionally substituted (C₁-C₃)alkyl. In certain cases, R²¹ is methyl. In certain cases, R²¹ is ethyl.

In certain cases of any one of formulae (I), (IIIA), (IIIB), or (IIIC), Z⁴ is a linking moiety selected from ester, amide, sulfonamide, urea, thiourea, amine, ether, thioether, optionally substituted aryl, optionally substituted heterocycle, optionally substituted spiroheterocycle, and optionally substituted heteroaryl. In certain cases, Z⁴ is a linking moiety selected from amide or amide bioisostere. In certain cases, Z⁴ is an amine. In certain cases, Z⁴ is an ether. In certain cases, Z⁴ is a thioether. In certain cases, Z⁴ is an optionally substituted aryl. In certain cases, Z⁴ is a 1,4-phenyl group. In certain cases, Z⁴ is an optionally substituted heteroaryl. In certain cases, Z⁴ is a oxadiazole. In certain cases, Z⁴ is a triazole. In certain cases, Z⁴ is an optionally substituted heterocycle. In certain cases, Z⁴ is a piperazine. In certain cases, Z⁴ is an optionally substituted spiroheterocycle. In certain cases, Z⁴ is 3,9-diazaspiro[5.5]undecane.

In certain cases, Z⁴ is an amide bioisostere. An amide bioisostere is a group with similar physical or chemical properties to an amide group. In certain cases, the amide bioisostere produces broadly similar biological properties to the corresponding amide group. In certain cases, the amide bioisostere may modify the activity of the compound, and may alter the metabolism of the compound. The subject compounds can include both acyclic and cyclic amide bioisosteres. Amide bioisosteres that can be utilized in the subject compounds includes, but is not limited to, imidazoles, triazoles, thiazoles, oxadiazoles, tetrazoles, indoles, olefins, fluoroalkenes, ureas, esters, thioamides, phosphonamidates, sulfonamides, trifluoro ethylamines, amidines, and carbamates. In some cases, the amide bioisostere is a 5-membered ring heterocycle, e.g., a triazole, an oxadiazole, an imidazole, a tetrazole, or a pyrazole. In certain cases, the amide bioisostere is a six membered heteroaryl, e.g., a pyrazine or a pyridine. In certain cases, the amide bioisostere is a retroinverted, or reverse amide, e.g., —NHC(O)— converted to —C(O)NH—. In certain cases, the amide bioisostere is a urea. In certain cases, the amide bioisostere is a carbamate. In certain cases, the amide bioisostere is an amidine. In certain cases, the amide bioisostere is a thioamide. In certain cases, the amide bioisostere is a trifluoroethylamine. In certain cases, the amide bioisostere is a sulfonamide. In certain cases, the amide bioisostere is a phosphonamidate. In certain cases, the amide bioisostere is an olefin. In certain embodiments, the amide bioisostere is a moiety as described in Kumari et al. 2020, J. Med. Chem., 63: 12290-12358. In certain embodiments, the amide bioisostere is a moiety of one of the following structures:

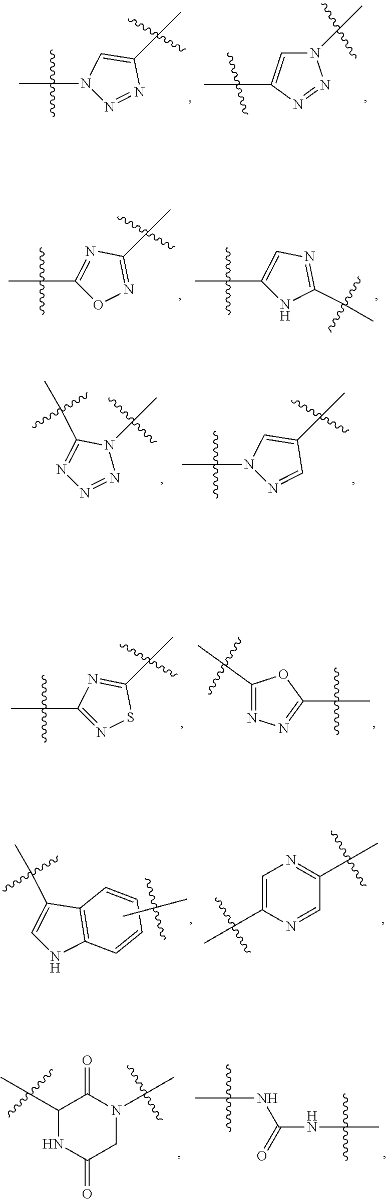

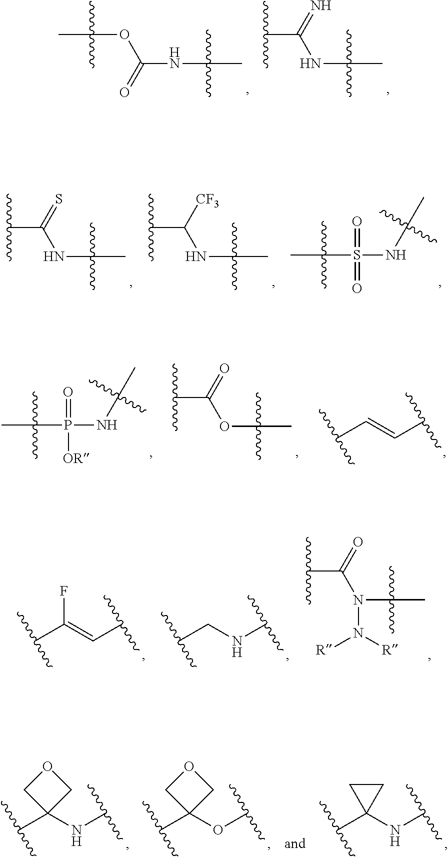

Where R″ is an optionally substituted (C₁-C₆)alkyl.

In certain cases, Z⁴ is a linking moiety selected from —CONR²¹—, —NR²¹—, —O—, —S—, optionally substituted aryl (e.g., 1,4-phenyl) and optionally substituted heteroaryl (e.g., oxadiazole or triazole), wherein R²¹ is selected from H, and optionally substituted (C₁-C₆)alkyl. In certain cases, R² is methyl. In certain cases, R²¹ is ethyl.

In some embodiments, Z⁴ is a linking group selected from:

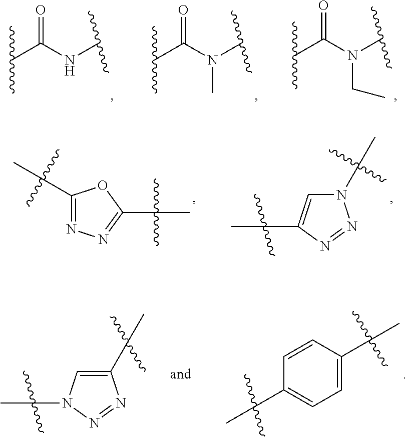

In some embodiments, Z⁴ is a linking group selected from:

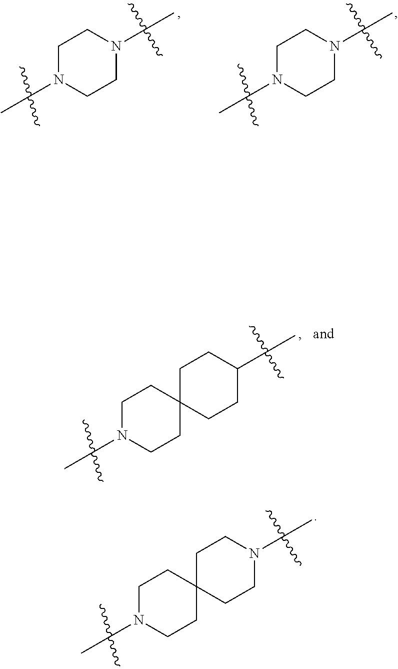 [SMILES: *N1CCN(*)CC1 ; *N1CCN(*)CC1 ; *C1CCC2(CC1)CCN(*)CC2 ; *N1CCC2(CC1)CCN(*)CC2]

In some embodiments of formula (I) or (IIIA), —Z²CH(-T³-Z³)T⁴Z⁴— is selected from the following structures:

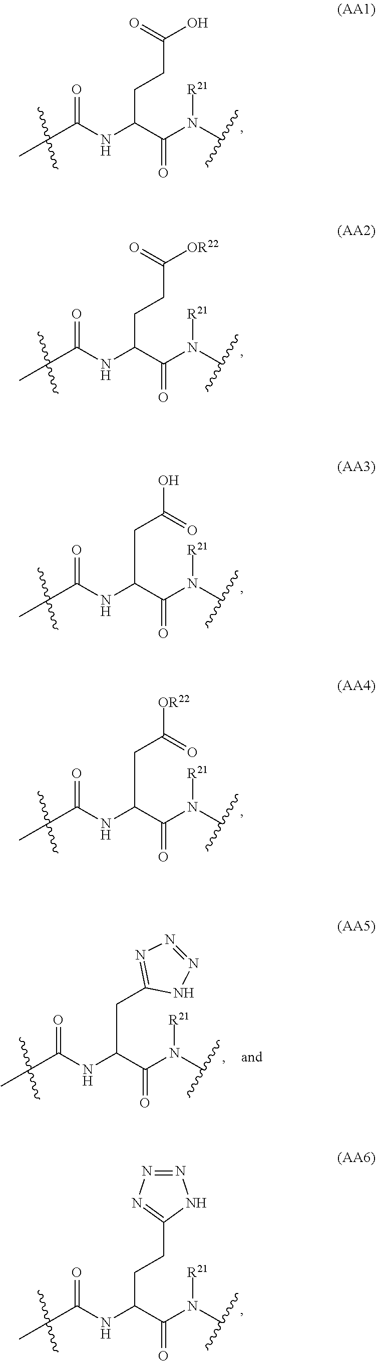

or a tautomer thereof, or a salt thereof.

In some embodiments of formula (I) or (IIIA), —Z²CH(-T³-Z³)T⁴Z⁴— is selected from the following structures:

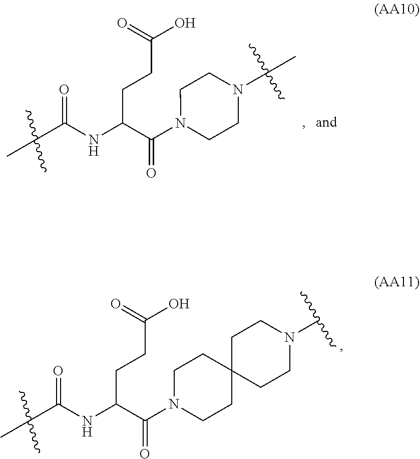 [SMILES: *C(=O)NC(CCC(=O)O)C(=O)N1CCN(*)CC1 ; *C(=O)NC(CCC(=O)O)C(=O)N1CCC2(CCN(*)CC2)CC1]

or a tautomer thereof, or a salt thereof.

In some embodiments of formula (I) or (IIIB), —Z²CH(-T³-Z³)T⁴Z⁴— of formula (I) is selected from the following structures:

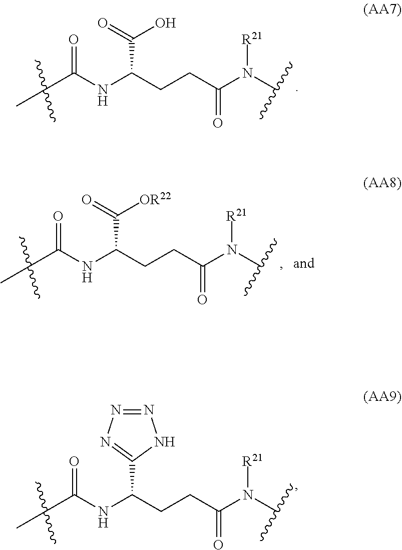

or a tautomer thereof, or a salt thereof.

In some embodiments of formula (I) or (IIIB), —Z²CH(-T³-Z³)T⁴Z⁴— of formula (I) is selected from the following structures:

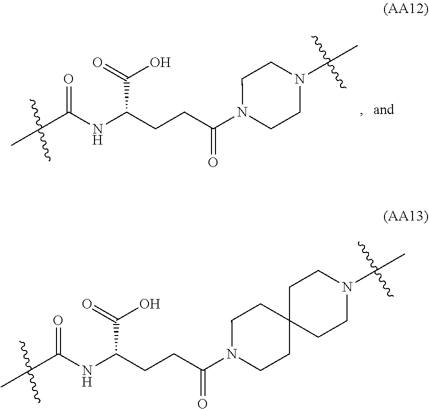 [SMILES: *C(=O)N[C@@H](CCC(=O)N1CCN(*)CC1)C(=O)O ; *C(=O)N[C@@H](CCC(=O)N1CCC2(CCN(*)CC2)CC1)C(=O)O]

or a tautomer thereof, or a salt thereof.

In some embodiments of formula (I) or (IIIC) —Z²CH(-T³-Z³)T⁴Z⁴— of formula (I) is selected from the following structures:

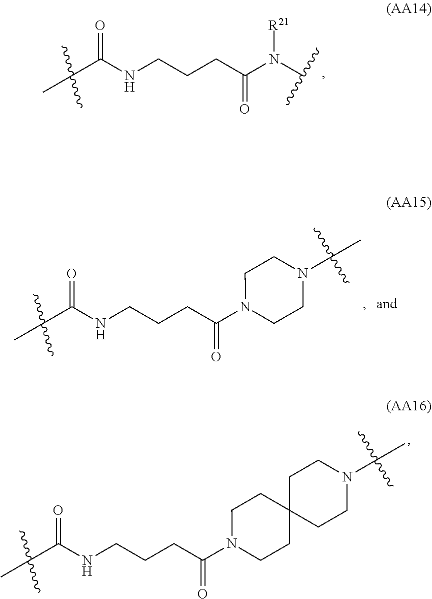

or a tautomer thereof, or a salt thereof.

In certain cases of (AA2), (AA4) or (AA8), R²² is optionally substituted (C₁-C₆)alkyl. In certain cases, R²² is methyl. In certain cases, R²² is ethyl. In some cases, R²² is propyl. In certain cases, R²² is substituted (C₁-C₆)alkyl. In certain cases, R²² is of the formula —(CH₂)mCH₂N(R²⁴)(R²⁵), where R²⁴ and R²⁵ are independently selected from H and optionally substituted (C₁-C₆)alkyl, or R²⁴ and R²⁵ are cyclically linked to provide an optionally substituted 5 or 6-membered heterocycle; and m is 1 to 5. In certain cases, R²⁴ and R²⁵ are H. In certain embodiments, R²⁴ and R²⁵ is optionally substituted (C₁₋₃)alkyl. In certain cases, R²⁴ and R²⁵ are cyclically linked to provide an optionally substituted 5-membered heterocycle. In certain other cases, R²⁴ and R²⁵ are cyclically linked to provide an optionally substituted 6-membered heterocycle. In certain cases, R²² is of the following structure:

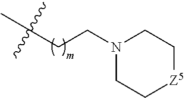

wherein
Z⁵ is O, NH or NR²¹; and R²¹ is (C₁-C₆)alkyl. In certain cases, Z⁵ is O and m is 1. In certain cases, Z⁵ is NH, and m is 1. In certain cases, Z⁵ is NCH₃, and m is 1.

In certain embodiments of any one of (AA1)-(AA9), R²¹ is H. In certain cases, R²¹ is methyl. In certain cases, R²¹ is ethyl. In certain cases, R²¹ is propyl. In certain cases, R²¹ is propargyl.

In some embodiments of formula (I) or (IIIA), —Z²CH(-T³-Z³)T⁴Z⁴— is of the structure (AA1). In certain cases, —Z²CH(-T³-Z³)T⁴Z⁴— is of the structure (AA2). In certain cases, —Z²CH(-T³-Z³)T⁴Z⁴— is of the structure (AA3). In certain cases, —Z²CH(-T³-Z³)T⁴Z⁴— is of the structure (AA4). In certain cases, —Z²CH(-T³-Z³)T⁴Z⁴— is of the structure (AA5). In certain cases, —Z²CH(-T³-Z³)T⁴Z⁴— is of the structure (AA6). In certain cases, —Z²CH(-T³-Z³)T⁴Z⁴— is of the structure (AA10). In certain cases, —Z²CH(-T³-Z³)T⁴Z⁴— is of the structure (AA11).

In certain embodiments of formula (I) or (IIIB), —Z²CH(-T³-Z³)T⁴Z⁴— is of the structure (AA7). In certain cases, —Z²CH(-T³-Z³)T⁴Z⁴— is of the structure (AA8). In certain other cases, —Z²CH(-T³-Z³)T⁴Z⁴— is of the structure (AA9). In certain other cases, —Z²CH(-T³-Z³)T⁴Z⁴— is of the structure (AA12). In certain other cases, —Z²CH(-T³-Z³)T⁴Z⁴— is of the structure (AA13).

In certain embodiments of formula (I) or (IIIC), —Z²CH(-T³-Z³)T⁴Z⁴— is of the structure (AA14). In certain other cases, —Z²CH(-T³-Z³)T⁴Z⁴— is of the structure (AA15). In certain other cases, —Z²CH(-T³-Z³)T⁴Z⁴— is of the structure (AA16).

In certain embodiments of the subject compounds, A¹ of ring system A is selected from —N═CR³—, —CR³═N—, or —CR³═CR³—. In certain cases, A¹ of ring system A is N═CR³—. In certain cases, A¹ of ring system A is —CR³═N—. In certain other cases, A¹ of ring system A is —CR³═CR³—.

In some embodiments of the subject compounds, A is of formula (IIA):

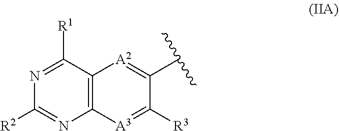

or a tautomer thereof, or a salt thereof, wherein:
A² is selected from N, and CR³;
A³ is independently selected from N, and CR²¹.

In certain embodiments of formula (IIA), A² and A³ are each N. In certain embodiments, A² is N and A³ is CR²¹. In certain cases, A² is CR³ and A³ is N. In certain other embodiments, A² and A³ are each independently CR³.

In certain embodiments of formula (IIA), each R³ is H. In certain other embodiments, R³ is halogen. In certain cases, the halogen is fluoride. In certain cases, R³ is OH. In certain cases, R³ is optionally substituted (C₁-C₆)alkyl. In certain cases, R³ is optionally substituted (C₁-C₆)alkoxy. In certain cases, R³ is COOH. In certain cases, R³ is NO₂. In certain cases, R³ is CN. In certain cases, R³ is NH₂, or —N(R²¹)₂. In certain cases, R³ is —OCOR²¹ or —COOR²¹. In certain other cases, R³ is —CONHR²¹, or —NHCOR²¹.

In certain embodiments of formula (IIA), R² is —NH₂. In certain embodiments, R² is optionally substituted (C₁-C₆)alkyl. In certain embodiments, R² is —CH₃. In certain embodiments, R² is —CH₂OH. In certain other embodiments, R² is H.

In certain embodiments of formula (IIA), R¹ is OH. In certain embodiments, R² is NH₂.

In certain embodiments of the subject compounds, A is selected from:

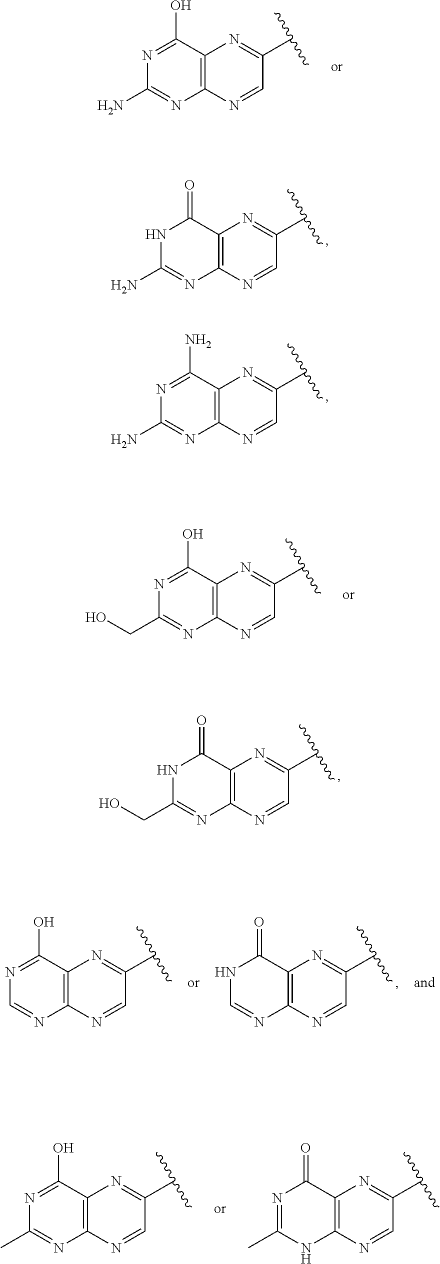

or a tautomer thereof.

In certain embodiments of the subject compounds, A¹ of ring system A is selected from —NR²¹—, —S—, —O— or —C(R²¹)₂—. In certain cases, A¹ of ring system A is —NR²¹—. In certain cases, A¹ of ring system A is —S—. In certain cases, A¹ of the ring system A is —O—. In certain other cases, A¹ of ring system A is —C(R²¹)₂—.

In some embodiments of the subject compounds, A is of formula (IIB) or (IIC):

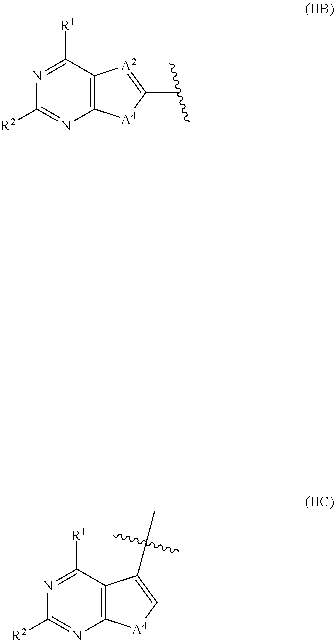

or a tautomer thereof, or a salt thereof, wherein A⁴ is selected from NR²¹, S, and O.

In certain embodiments of formula (IIB), A² is CR³. In certain cases, A² is N. In certain cases of formula (IIB), A⁴ is NR²¹. In certain cases, A⁴ is S. In certain other embodiments, A⁴ is O. In certain embodiments, A² is CR³ and A⁴ is NR²¹.

In certain embodiments of formula (IIB), each R³ is H. In certain other embodiments, R³ is halogen. In certain cases, the halogen is fluoride. In certain cases, R³ is OH. In certain cases, R³ is optionally substituted (C₁-C₆)alkyl. In certain cases, R³ is optionally substituted (C₁-C₆)alkoxy. In certain cases, R³ is COOH. In certain cases, R³ is NO₂. In certain cases, R³ is CN. In certain cases, R³ is NH₂, or —N(R²¹)₂. In certain cases, R³ is —OCOR²¹ or —COOR²¹. In certain other cases, R³ is —CONHR²¹, or —NHCOR²¹.

In certain embodiments of formula (IIB), R² is —NH₂. In certain embodiments, R² is optionally substituted (C₁-C₆)alkyl. In certain embodiments, R² is —CH₃. In certain embodiments, R² is —CH₂OH. In certain other embodiments, R² is H.

In certain embodiments of formula (IIB), R¹ is OH. In certain embodiments, R² is NH₂.

In certain embodiments of the subject compounds, A is selected from:

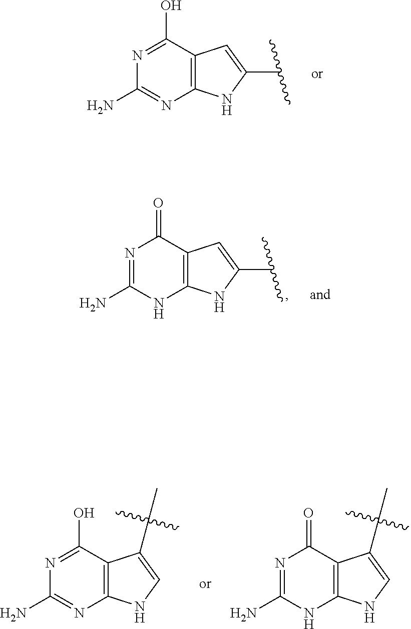 [SMILES: *c1cc2c(O)nc(N)nc2[nH]1 ; *c1cc2c(=O)nc(N)[nH]c2[nH]1 ; *c1c[nH]c2nc(N)nc(O)c12 ; *c1c[nH]c2[nH]c(N)nc(=O)c12]

or a tautomer thereof.

In certain embodiments of any one of formulae (I), (IIIA) or (IIIB), T¹ is CH₂. In certain embodiments, T¹ is CH₂CH₂. In certain other embodiments, T¹ is CH₂CH₂CH₂.

In certain embodiments of any one of formulae (I), (IIIA) or (IIIB), Z¹ is NR¹. In certain cases, R²¹ is H. In certain cases, R²¹ is methyl. In certain cases, R²¹ is ethyl. In certain cases, R²¹ is propyl. In certain cases, R²¹ is propargyl.

In certain cases of any one of formulae (I), (IIIA) or (IIIB), Z¹ is O. In certain other cases, Z¹ is S.

In certain cases of any one of formulae (I), (IIIA) or (IIIB), Z¹ is substituted methylene. In certain cases of any one of formulae (I), (IIIA) or (IIIB), Z¹ is methylene substituted with propargyl (i.e., —CH(propargyl)-. In certain cases of any one of formulae (I), (IIIA) or (IIIB), Z¹ is methylene substituted with (C₁-C₃)alkyl.

In certain embodiments of any one of formulae (I), (IIIA) or (IIIB), T¹-Z¹ is optionally substituted (C₁-C₆)alkylene. In certain cases, T¹-Z¹ is —CH₂CH₂—. In certain cases, T¹-Z¹ is —CH₂CH₂CH₂CH₂—. In certain cases, T¹-Z¹ is —CH₂CH₂CH₂—. In certain embodiments of any one of formulae (I), (IIIA) or (IIIB), T¹-Z¹ is —CH₂CH(propargyl)-.

In certain embodiments of the subject compounds, the B ring system is an optionally substituted aryl. In certain cases, the B ring system is an optionally substituted heteroaryl. In certain cases, the B ring system is an optionally substituted heterocycle. In certain cases, the B ring system is an optionally substituted cycloalkyl. In certain other cases, the B ring system is an optionally substituted bridged bicycle.

In certain embodiments of the subject compounds, the B ring system is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, optionally substituted indole, and optionally substituted bicycloalkyl (e.g., bicyclo[1.1.1]pentane).

In certain embodiments of the subject compounds, the B ring system is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

In certain embodiments of the subject compounds, B—Z² is selected from any one of formulae (BZ1)-(BZ8):

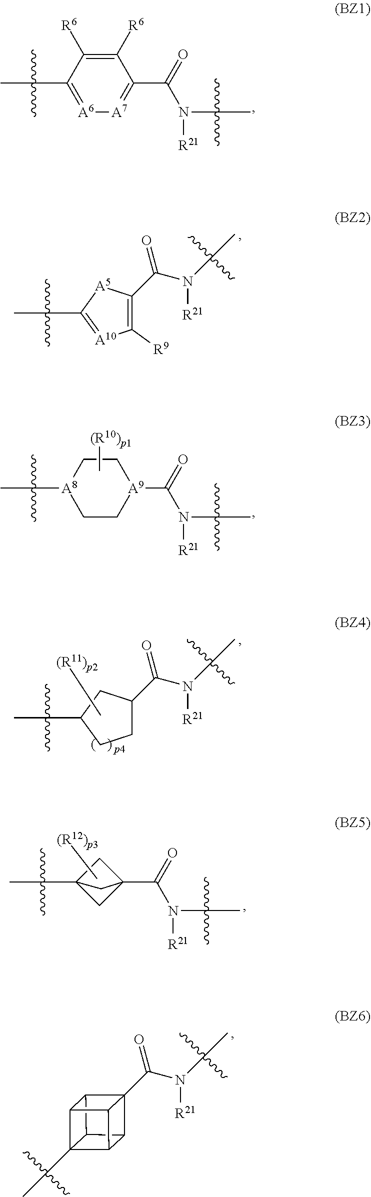

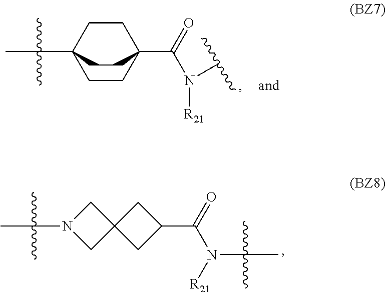

wherein:
A⁵ is selected from NR²¹, S, O, C(R⁵)₂;
A⁶-A⁹ are independently selected from N, and CR⁵; A¹⁰ is selected from N, and CR⁸;
R²¹ is selected from H, and optionally substituted (C₁-C₆)alkyl;
each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

In certain embodiments of the subject compounds, B—Z² is of formula (BZ1). In certain embodiments of formula (BZ1), each A⁶ and A⁷ is CR⁵. In certain cases, at least one of A⁶ and A⁷ is N. In certain cases, A⁶ is CR⁵ and A⁷ is N. In certain other cases, A⁶ is N and A⁷ is CR⁵. In certain cases, R⁵ is H. In certain cases, R is halogen. In certain cases, the halogen is F or Cl. In certain cases, R⁵ is (C₁-C₃)alkyl. In certain cases, R is methyl. In certain cases, each of R⁶ and R⁷ is H. In certain other cases, at least one of R⁶ and R⁷ is a substituent other than H. In certain cases, at least one of R⁶ and R⁷ is halogen. In certain cases, the halogen is F or Cl. In certain cases, at least one of R⁶ and R⁷ is (C₁-C₃)alkyl. In certain cases, at least one of R⁶ and R⁷ is methyl. In certain embodiments of formula (BZ1), R²¹ is H. In certain other embodiments, R²¹ is (C₁-C₃)alkyl. In certain cases, R²¹ is methyl.

In certain embodiments of the subject compounds, B—Z² is of formula (BZ2). In certain embodiments of formula (BZ2), A⁵ is NR²¹, where R²¹ is selected from H or (C₁-C₃)alkyl, e.g., methyl. In certain cases, A⁵ is S. In certain cases, A⁵ is O. In certain other cases, A⁵ is C(R⁵)₂. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, A¹⁰ is CR⁸ and each of R⁸ and R⁹ is H. In certain other cases, A¹⁰ is CR⁸ and at least one of R⁸ and R⁹ is a substituent other than H. In certain cases, A¹⁰ is CR⁸ and at least one of R⁸ and R⁹ is halogen. In certain cases, the halogen is F or Cl. In certain cases, at least one of R⁸ and R⁹ is (C₁-C₃)alkyl. In certain cases, A¹⁰ is CR⁸ and at least one of R⁸ and R⁹ is methyl. In certain embodiments of formula (BZ2), R²¹ is H. In certain other embodiments, R²¹ is (C₁-C₃)alkyl. In certain cases, R²¹ is methyl. In certain embodiments of formula (BZ2), A¹⁰ is CR⁸, where R⁸ is selected from H or (C₁-C₃)alkyl, e.g., methyl. In certain embodiments of formula (BZ2), A¹⁰ is CH. In cases of formula (BZ2), A¹⁰ is N. In certain embodiments of formula (BZ2), A⁵ is NR²¹ and A¹⁰ is CR⁸, where R²¹ and R⁸ are independently selected from H or (C₁-C₃)alkyl, e.g., methyl. In certain embodiments of formula (BZ2), A⁵ is NR²¹ and A¹⁰ is N. In certain embodiments of formula (BZ2), A⁵ is S and A¹⁰ is N.

In certain embodiments of the subject compounds, B—Z² is of formula (BZ3). In certain embodiments of formula (BZ3), each A⁸ and A⁹ is CR⁵. In certain cases, at least one of A⁸ and A⁹ is N. In certain cases, A⁸ is CR⁵ and A⁹ is N. In certain other cases, A⁸ is N and A⁹ is CR⁵. In certain cases, both of A⁸ and A⁹ are N. In certain cases, R⁵ is H. In certain cases, R⁵ is halogen. In certain cases, the halogen is F or Cl. In certain cases, R⁵ is (C₁-C₃)alkyl. In certain cases, R⁵ is methyl. In certain cases, each R¹⁰ is H (or p1 is 0). In certain other cases, p1 is 1 to 10 and at least one R¹⁰ group is a substituent other than H. In certain cases, at least one R¹⁰ group is halogen. In certain cases, the halogen is F or Cl. In certain cases, at least one R¹⁰ group is (C₁-C₃)alkyl. In certain cases, at least one of R¹⁰ group is methyl. In certain embodiments of formula (BZ3), R²¹ is H. In certain other embodiments, R²¹ is (C₁-C₃)alkyl. In certain cases, R²¹ is methyl.

In certain embodiments of the subject compounds, B—Z² is of formula (BZ4). In certain embodiments of formula (BZ4), p4 is 0, such that the B ring system is cyclobutyl. In certain cases, p4 is 1, such that the B ring system is a cyclopentyl. In certain cases, p4 is 2, such that the B ring system is cyclohexyl. In certain cases, p4 is 3, such that the B ring system is cycloheptyl. In certain other cases, p4 is 4, such that the B ring system is cyclooctyl. In certain cases, each R¹¹ is H (or p2 is 0). In certain other cases, p2 is 1 to 14 and at least one R¹¹ group is a substituent other than H. In certain cases, at least one R¹¹ group is halogen. In certain cases, the halogen is F or Cl. In certain cases, at least one R¹¹ group is (C₁-C₃)alkyl. In certain cases, at least one of R¹¹ group is methyl. In certain embodiments of formula (BZ4), R²¹ is H. In certain other embodiments, R²¹ is (C₁-C₃)alkyl. In certain cases, R²¹ is methyl.

In certain embodiments of the subject compounds, B—Z² comprises a bicycloalkyl group and is of any of formulae (BZ5)-(BZ8). In certain embodiments of formula (BZ5), each R¹² is H (or p3 is 0). In certain other cases, p3 is 1 to 4 and at least one R¹² group is a substituent other than H. In certain cases, at least one R¹² group is halogen. In certain cases, the halogen is F or Cl. In certain cases, at least one R¹² group is (C₁-C₃)alkyl. In certain cases, at least one of R¹² group is methyl. In certain embodiments of formula (BZ5), R²¹ is H. In certain other embodiments, R²¹ is (C₁-C₃)alkyl. In certain cases, R²¹ is methyl. In certain embodiments of any of formulae (BZ6)-(BZ8), R²¹ is H. In certain other embodiments, R²¹ is (C₁-C₃)alkyl. In certain cases, R²¹ is methyl.

In certain embodiments of the subject compounds, B—Z² is:

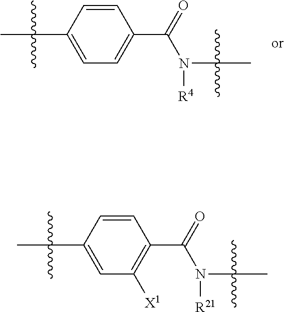

wherein X¹ is halogen. In certain cases, the halogen is F. In certain cases, the halogen is Cl. In certain cases, the halogen is bromide.

In certain embodiments of the subject compounds, T¹-Z¹—B is selected from:

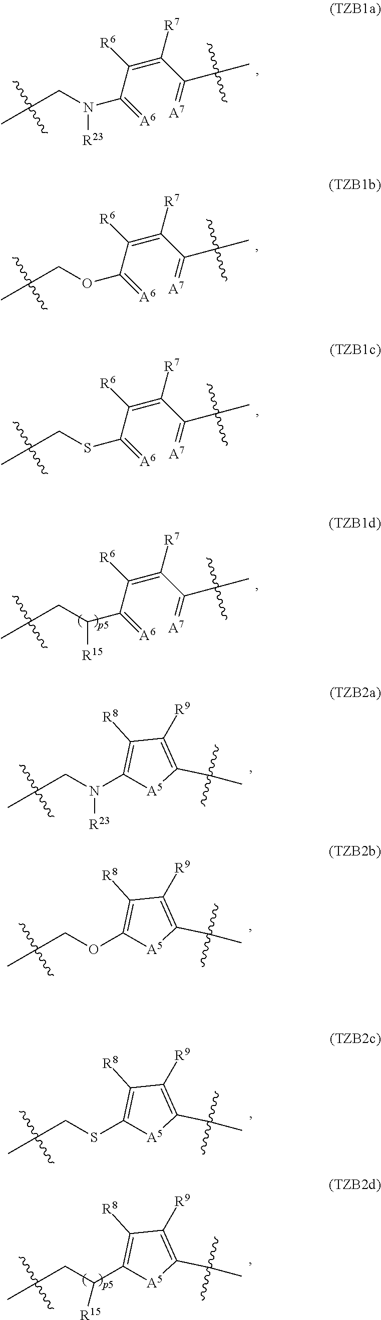

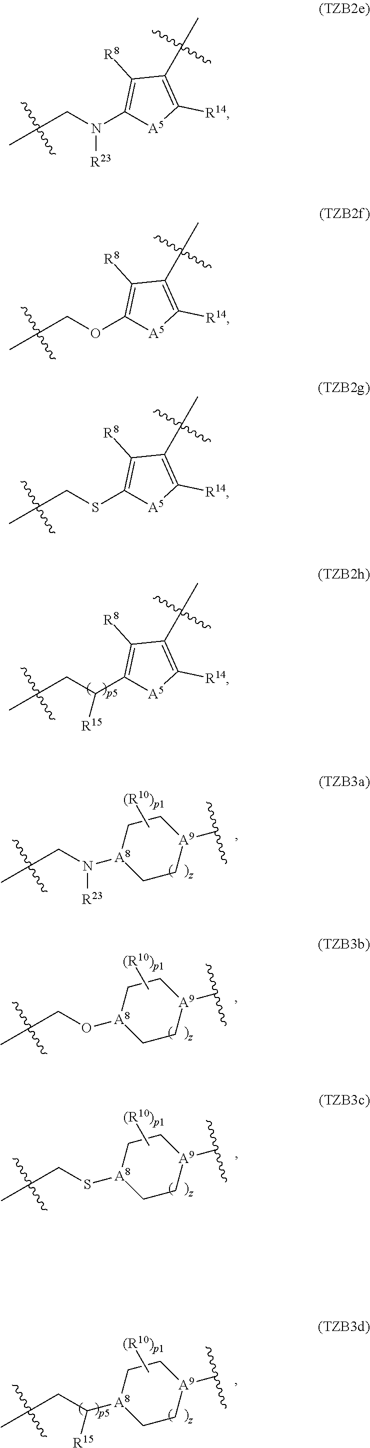

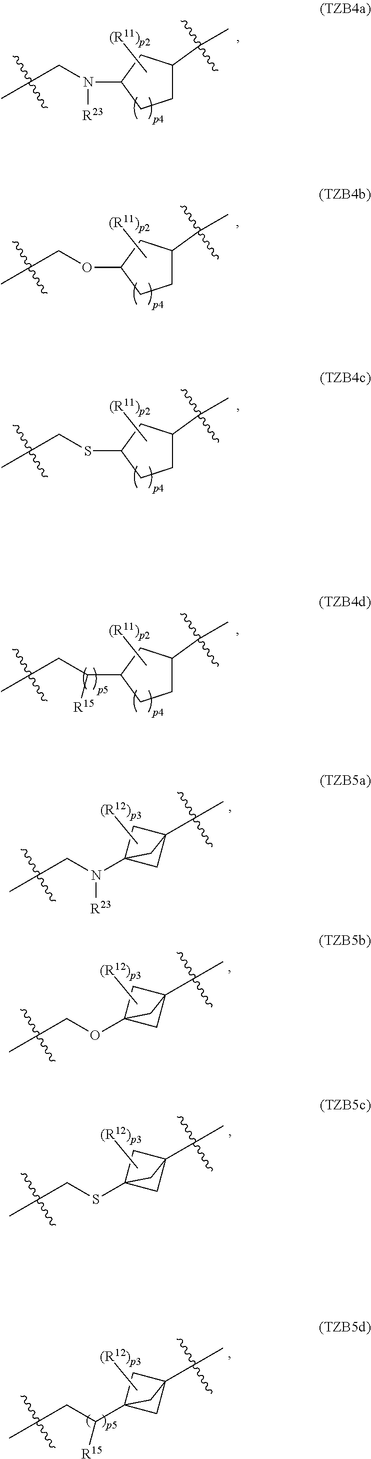

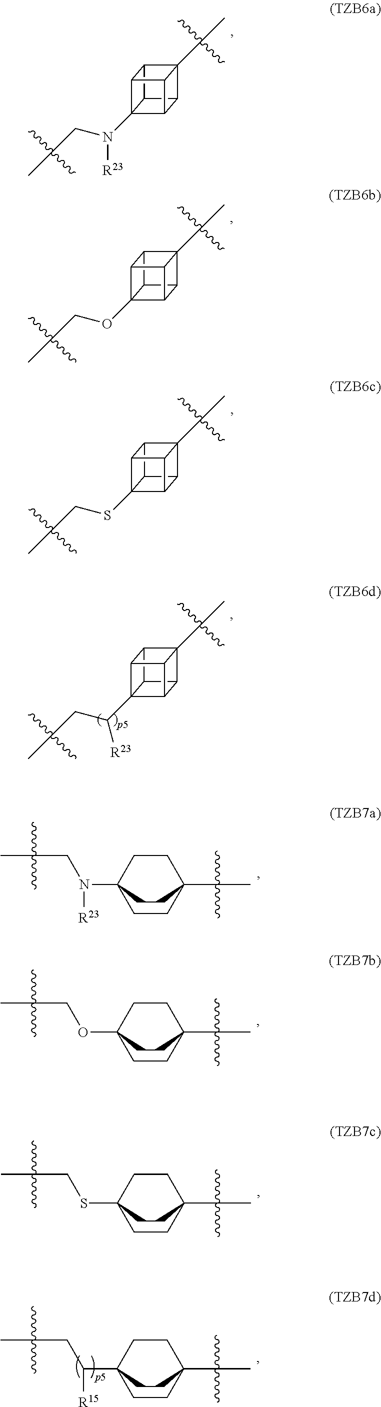

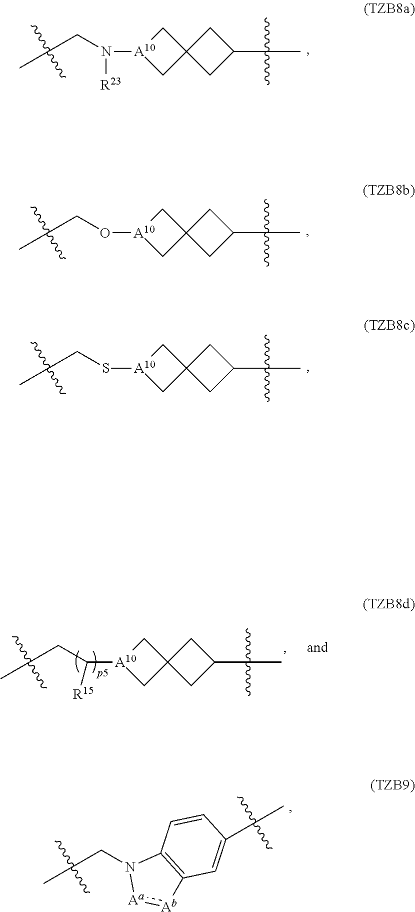

wherein:
A⁵ is selected from NR²¹, S, O, C(R⁵)₂;
A⁶-A¹⁰ are independently selected from N, and CR⁵;
R²³ is H, optionally substituted (C₁-C₆)alkyl, or R²³ forms a 5 or 6 membered cycle together with an atom of the adjacent cycle;
each R⁵ to R¹² and R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
R¹⁵ is H, optionally substituted (C₁-C₆)alkyl, or R¹⁵ forms a 5 or 6 membered cycle together with an atom of the adjacent cycle;
[symbol] is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N
p1 is 0 to 10;
p2 is 0 to 14;
p3 is 0 to 4;
p4 0 to 4; and
p5 is 1 to 3.

In certain embodiments of the subject compounds, T¹-Z¹—B is of any one of formulae (TZB1a)-(TZB1d), and each of A⁶-A⁷, and R⁶-R⁷ are as defined for formula (BZ1). In certain embodiments of formula (TZB1a) or (TZB1d), R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent aryl or heteroaryl ring. In certain embodiments R²³ or R¹⁵ forms a fused 6-membered cycle with an atom of the adjacent aryl or heteroaryl ring. In certain embodiments of formula (TZB1d), p5 is 1. In certain embodiments, p5 is 2. In certain other embodiments, p5 is 3.

In certain embodiments of the subject compounds, T¹-Z¹—B is of any one of formulae (TZB2a)-(TZB2 h), and each of A⁵, and R⁸-R⁹ are as defined for formula (BZ2). In certain embodiments, R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent 5-membered ring. In certain embodiments R²³ or R¹⁵ forms a fused 6-membered cycle with an atom of the adjacent 5-membered ring. In certain embodiments of formula (TZB2d) or (TZB2 h), p5 is 1. In certain embodiments, p5 is 2. In certain other embodiments, p5 is 3.

In certain embodiments of the subject compounds, T¹-Z¹—B is of any one of formulae (TZB3a)-(TZB3d), and each of A⁸-A⁹, R¹⁰, z and p1 are as defined for formula (BZ3). In certain embodiments of formula (TZB3a) or (TZB3d), R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent 6-membered ring. In certain embodiments R²³ or R¹⁵ forms a fused 6-membered cycle with an atom of the adjacent 6-membered ring. In certain embodiments of formula (TZB3d), p5 is 1. In certain embodiments, p5 is 2. In certain other embodiments, p5 is 3.

In certain embodiments of the subject compounds, T¹-Z¹—B is of any one of formulae (TZB4a)-(TZB4d), and each of R¹, p2 and p4 are as defined for formula (BZ4). In certain embodiments of formula (TZB4a) or (TZB4d), R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments R²³ or R¹⁵ forms a fused 5-membered cycle with an atom of the adjacent ring. In certain embodiments R²³ or R¹⁵ forms a fused 6-membered cycle with an atom of the adjacent ring. In certain embodiments of formula (TZB4d), p5 is 1. In certain embodiments, p5 is 2. In certain other embodiments, p5 is 3.

In certain embodiments of the subject compounds, T¹-Z¹—B is selected from any one of formulae (TZB5a)-(TZB5d), (TZB6a)-(TZB6d), (TZB7a)-(TZB7d), and (TZB8a)-(TZB8d), and each of R¹², and p3 are as defined for formula (BZ5). In certain embodiments R²³ or R¹⁵ is H. In certain other embodiments, R²³ or R¹⁵ is optionally substituted (C₁-C₃)alkyl. In certain cases, R²³ or R¹⁵ is methyl. In certain embodiments, R²³ or R¹⁵ is an alkyne moiety of formula —(CH₂)nCCH, where n is 1 or 2. In certain embodiments of formula (TZB4d), (TZB6d), (TZB7d), or (TZB8d), p5 is 1. In certain embodiments, p5 is 2. In certain other embodiments, p5 is 3.

In certain embodiments of the subject compounds, T¹-Z¹—B is of formula (TZB9). In certain cases, the compound of formula (TZB9) is of any one of the following structures:

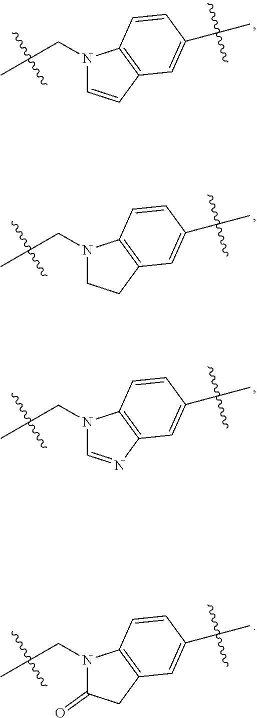 [SMILES: *Cn1ccc2cc(*)ccc21 ; *CN1CCc2cc(*)ccc21 ; *Cn1cnc2cc(*)ccc21 ; *CN1C(=O)Cc2cc(*)ccc21]

In certain embodiments of the subject compounds, T¹-Z¹ is optionally substituted (C₁-C₆)alkylene, and A-T¹-Z¹—B— is selected from one of formulae (AB1)-(AB6):

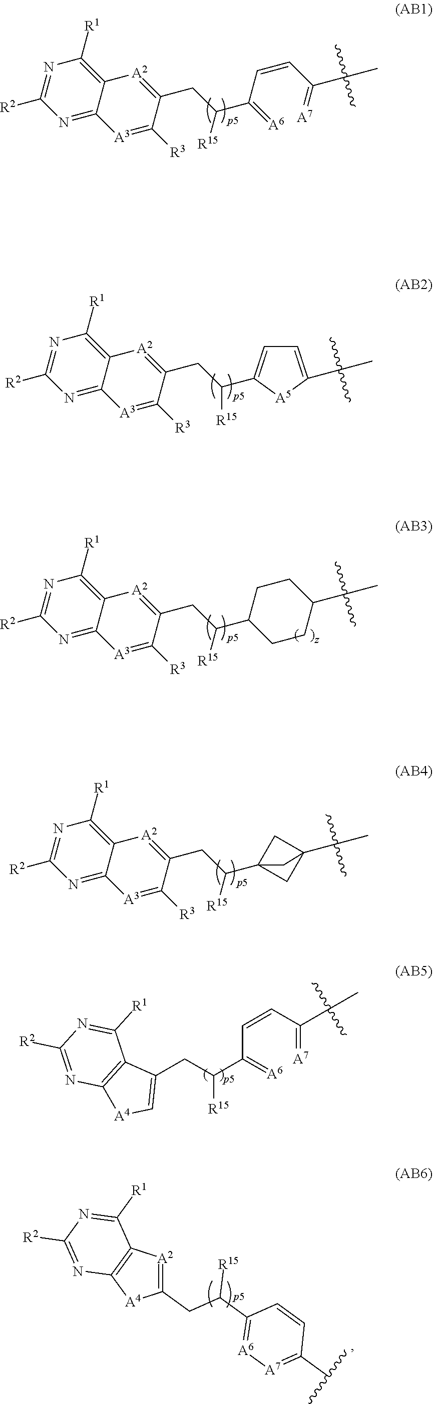

or a tautomer thereof, wherein:
A²-A⁷, R¹-R³ and z are as described herein above;
each R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

In certain embodiments of the subject compounds, A-T¹-Z¹—B— is of formula (AB1), and each of A²-A³, A⁶-A⁷, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain instances, both A⁶ and A⁷ are CH. In certain embodiments of formula (AB1), R⁵ is H. In certain other embodiments, R¹⁵ is optionally substituted (C₁-C₃)alkyl. In certain cases, R⁵ is methyl. In certain embodiments, R¹⁵ is an alkyne moiety of formula —(CH₂)nCCH, where n is 1 or 2. In certain embodiments R⁵ forms a fused 5-membered cycle with an atom of the adjacent aryl or heteroaryl ring. In certain embodiments R¹⁵ forms a fused 6-membered cycle with an atom of the adjacent aryl or heteroaryl ring. In certain embodiments of formula (AB1), p5 is 1. In certain embodiments, p5 is 2. In certain other embodiments, p5 is 3.

In certain embodiments of formula (AB1), the compound is selected from one of the following:

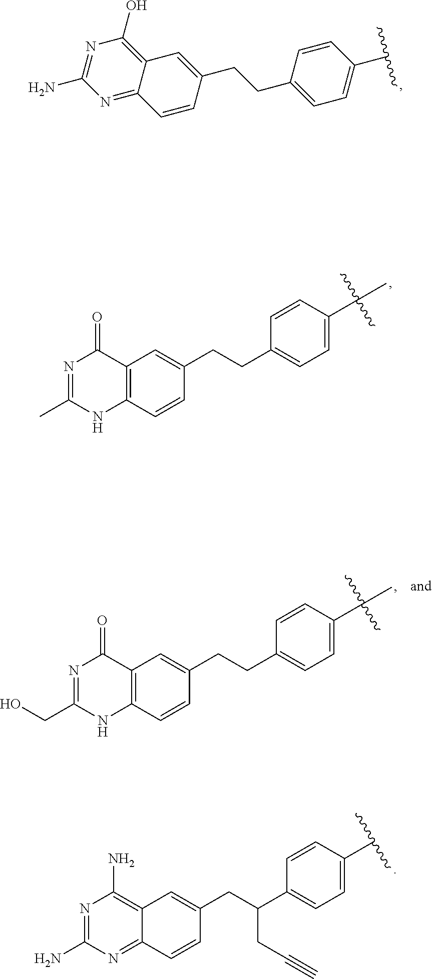 [SMILES: *c1ccc(CCc2ccc3nc(N)nc(O)c3c2)cc1 ; *c1ccc(CCc2ccc3[nH]c(C)nc(=O)c3c2)cc1 ; *c1ccc(CCc2ccc3[nH]c(CO)nc(=O)c3c2)cc1 ; *c1ccc(C(CC#C)Cc2ccc3nc(N)nc(N)c3c2)cc1]

In certain embodiments of the subject compounds, A-T¹-Z¹—B— is of formula (AB2), and each of A²-A³, A⁵, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain instances, A⁵ is S or O. In certain embodiments of formula (AB2), R⁵ is H. In certain other embodiments, R¹⁵ is optionally substituted (C₁-C₃)alkyl. In certain cases, R¹⁵ is methyl. In certain embodiments, R⁵ is an alkyne moiety of formula —(CH₂)nCCH, where n is 1 or 2. In certain embodiments R¹⁵ forms a fused 5-membered cycle with an atom of the adjacent 5-membered ring. In certain embodiments R⁵ forms a fused 6-membered cycle with an atom of the adjacent 5-membered ring. In certain embodiments of formula (AB2), p5 is 1. In certain embodiments, p5 is 2. In certain other embodiments, p5 is 3.

In certain embodiments of the subject compounds, A-T¹-Z¹—B— is of formula (AB3), and each of A²-A³, R¹-R³ and z are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain instances, z is 1. In certain embodiments of formula (AB3), R¹⁵ is H. In certain other embodiments, R¹⁵ is optionally substituted (C₁-C₃)alkyl. In certain cases, R⁵ is methyl. In certain embodiments, R¹⁵ is an alkyne moiety of formula —(CH₂)nCCH, where n is 1 or 2. In certain embodiments R⁵ forms a fused 5-membered cycle with an atom of the adjacent cycloalkyl ring. In certain embodiments R⁵ forms a fused 6-membered cycle with an atom of the adjacent cycloalkyl ring. In certain embodiments of formula (AB1), p5 is 1. In certain embodiments, p5 is 2. In certain other embodiments, p5 is 3.

In certain embodiments of formula (AB3), the compound is of the following structure:

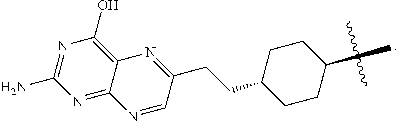 [SMILES: C[C@H]1CC[C@H](CCc2cnc3nc(N)nc(O)c3n2)CC1]

In certain embodiments of the subject compounds, A-T¹-Z¹—B— is of formula (AB4), and each of A²-A³, and R¹-R³ are as described herein. In certain instances, R¹ is OH or NH₂. In certain instances, R² is NH₂, CH₃, or CH₂OH. In certain instances, R³ is H. In certain instances, both A² and A³ are N. In certain other instances, both A² and A³ are CH. In certain embodiments of formula (AB4), R⁵ is H. In certain other embodiments, R¹⁵ is optionally substituted (C₁-C₃)alkyl. In certain cases, R⁵ is methyl. In certain embodiments, R¹⁵ is an alkyne moiety of formula —(CH₂)nCCH, where n is 1 or 2. In certain embodiments of formula (AB4), p5 is 1. In certain embodiments, p5 is 2. In certain other embodiments, p5 is 3.

In certain embodiments of the subject compounds, A-T¹-Z¹—B— is of formula (AB5) or (AB6), and each of A², A⁴, A⁶-A⁷, and R¹-R² are as described herein. In certain instances, R¹ is OH or NH₂. In certain instances, R² is NH₂, CH₃, or CH₂OH. In certain instances of formula (AB6), A² is CH. In certain other instances of formula (AB5) and (AB6), A⁴ is NH. In certain instances, both A⁶ and A⁷ are CH. In certain instances, A⁶ is CH and A⁷ are N. In certain embodiments of formula (AB5) or (AB6), R¹⁵ is H. In certain other embodiments, R¹ is optionally substituted (C₁-C₃)alkyl. In certain cases, R¹⁵ is methyl. In certain embodiments, R⁵ is an alkyne moiety of formula —(CH₂)nCCH, where n is 1 or 2. In certain embodiments R¹⁵ forms a fused 5-membered cycle with an atom of the adjacent aryl or heteroaryl ring. In certain embodiments R¹⁵ forms a fused 6-membered cycle with an atom of the adjacent aryl or heteroaryl ring. In certain embodiments of formula (AB5) or (AB6), p5 is 1. In certain embodiments, p5 is 2. In certain other embodiments, p5 is 3.

In certain embodiments, the compound of formula (AB5) or (AB6) is selected from the following structures:

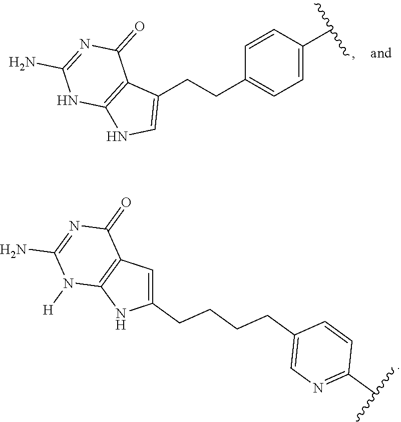 [SMILES: *c1ccc(CCc2c[nH]c3[nH]c(N)nc(=O)c23)cc1 ; *c1ccc(CCCCc2cc3c(=O)nc(N)[nH]c3[nH]2)cn1]

In certain embodiments of the subject compounds, A-T¹-Z¹—B— is selected from one of formulae (AB7)-(AB12):

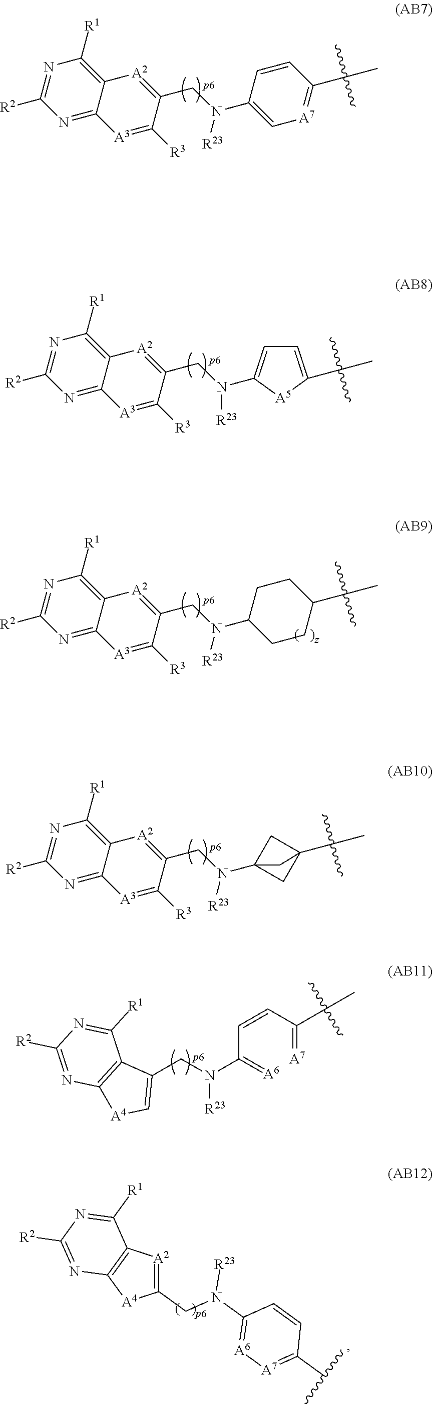

or a tautomer thereof, wherein:
A²-A⁷, R¹-R³ and z are as described herein above;
R²³ is H, optionally substituted (C₁-C₆)alkyl, or R²³ forms a 5 or 6 membered cycle together with an atom of the adjacent cycle;
each p6 is independently 1 to 3.

In certain embodiments of formula (AB7) to (AB12), R²³ is H. In certain other embodiments, R²³ is optionally substituted (C₁-C₃)alkyl. In certain cases, R²³ is methyl. In certain embodiments, R²³ is an alkyne moiety of formula —(CH₂)nCCH, where n is 1 or 2. In certain embodiments R²³ forms a fused 5-membered cycle with an atom of the adjacent aryl or heteroaryl ring. In certain embodiments R²³ forms a fused 6-membered cycle with an atom of the adjacent aryl or heteroaryl ring. In certain embodiments of formula (AB7) to (AB12), p6 is 1. In certain embodiments, p6 is 2. In certain other embodiments, p6 is 3.

In certain embodiments of the subject compounds, A-T¹-Z¹—B— is selected from one of formulae (AB13)-(AB18):

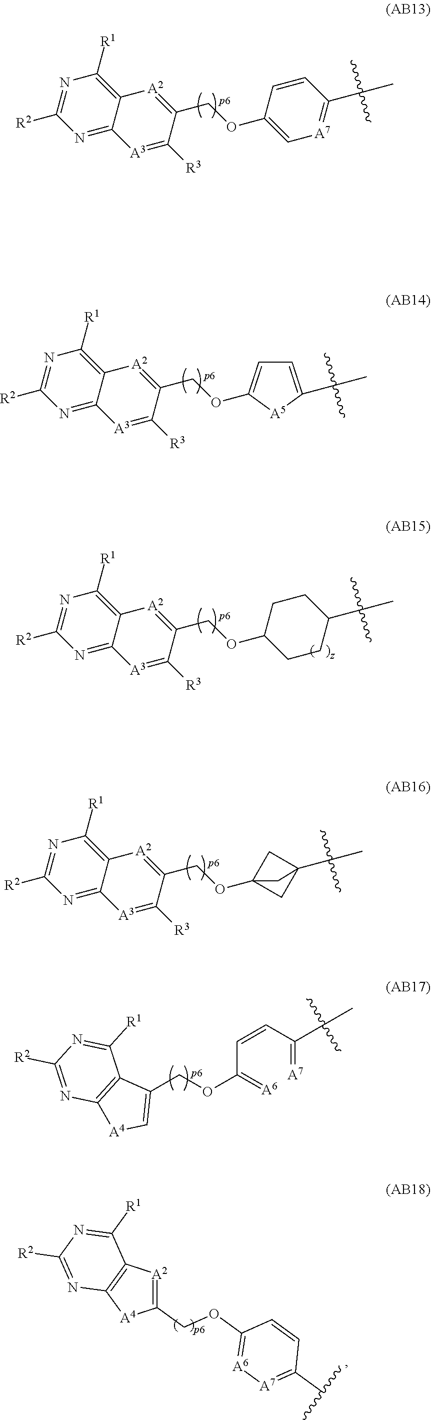

or a tautomer thereof, wherein:
A²-A⁷, R¹-R³ and z are as described herein above; and
each p6 is independently 1 to 3.

In certain embodiments of formula (AB13) to (AB18), p6 is 1. In certain embodiments, p6 is 2. In certain other embodiments, p6 is 3.

In certain embodiments of the subject compounds, A-T¹-Z¹—B— is selected from one of formulae (AB19)-(AB24):

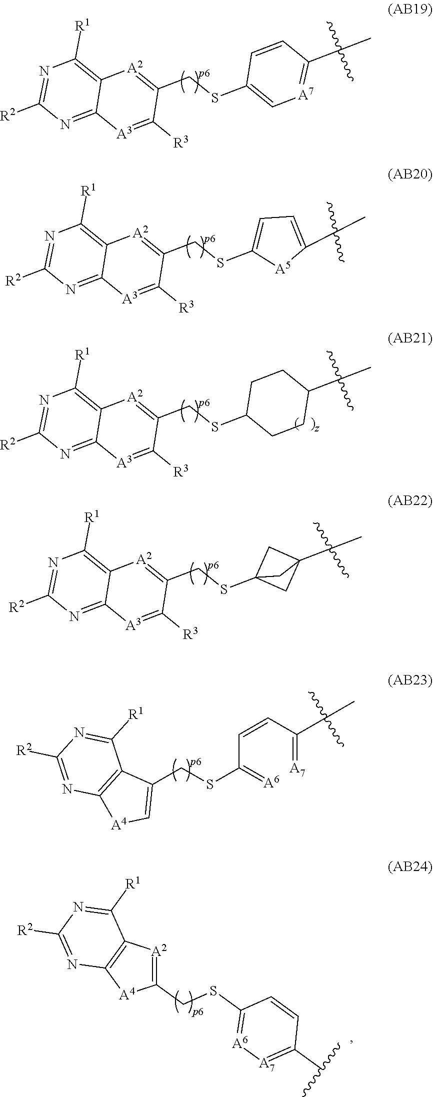

or a tautomer thereof, wherein:
A²-A⁷, R¹-R³ and z are as described herein above; and
each p6 is independently 1 to 3.

In certain embodiments of formula (AB19) to (AB24), p6 is 1. In certain embodiments, p6 is 2. In certain other embodiments, p6 is 3.

In some embodiments, the subject compound comprises a cell surface folate receptor ligand selected from one of the following structures:

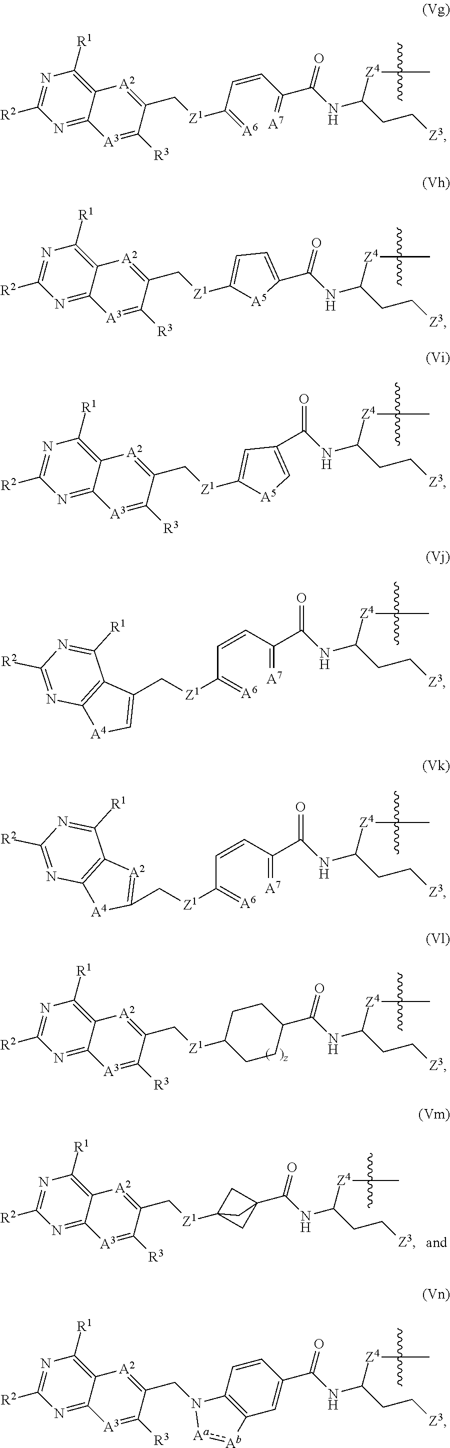

wherein:
A⁵ is selected from NR²¹, S, O, C(R⁵)₂;
A⁶ and A⁷ are independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

In certain embodiments, the subject compound comprises a cell surface folate receptor ligand selected from one of the following structures:

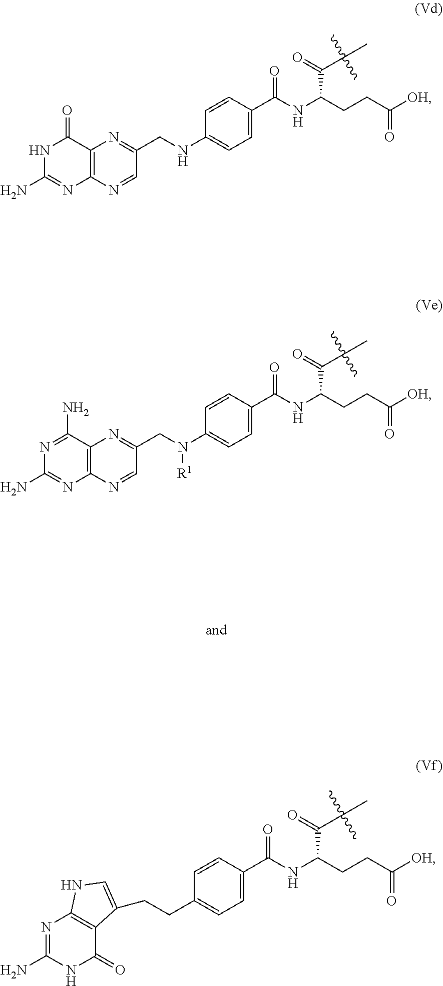

wherein R¹ is —H or —CH₃.

In certain embodiments, the subject compound comprises a cell surface folate receptor ligand is of formula (Vg) and each of R¹-R³, A²-A³, A⁶-A⁷, Z¹ and Z³-Z⁴ are as described herein above.

In certain embodiments, the subject compound comprises a cell surface folate receptor ligand is of formula (Vh) or (Vi) and each of R¹-R³, A²-A³, A⁵, Z¹ and Z³-Z⁴ are as described herein above.

In certain embodiments, the subject compound comprises a cell surface folate receptor ligand is of formula (Vj) or (Vk) and each of R¹-R², A², A⁴, A⁶-A⁷, Z¹ and Z³-Z⁴ are as described herein above.

In certain embodiments, the subject compound comprises a cell surface folate receptor ligand is of formula (Vl) and each of R¹-R³, A²-A³, z, Z¹ and Z³-Z⁴ are as described herein above.

In certain embodiments, the subject compound comprises a cell surface folate receptor ligand is of formula (Vm) and each of R¹-R³, A²-A³, Z¹ and Z³-Z⁴ are as described herein above.

In certain embodiments, the subject compound comprises a cell surface folate receptor ligand is of formula (Vn) and each of R¹-R³, A²-A³, A^{a}-A^{b}, and Z³-Z⁴ are as described herein above.

In some embodiments, the subject compound comprises a cell surface folate receptor ligand selected from one of the following structures:

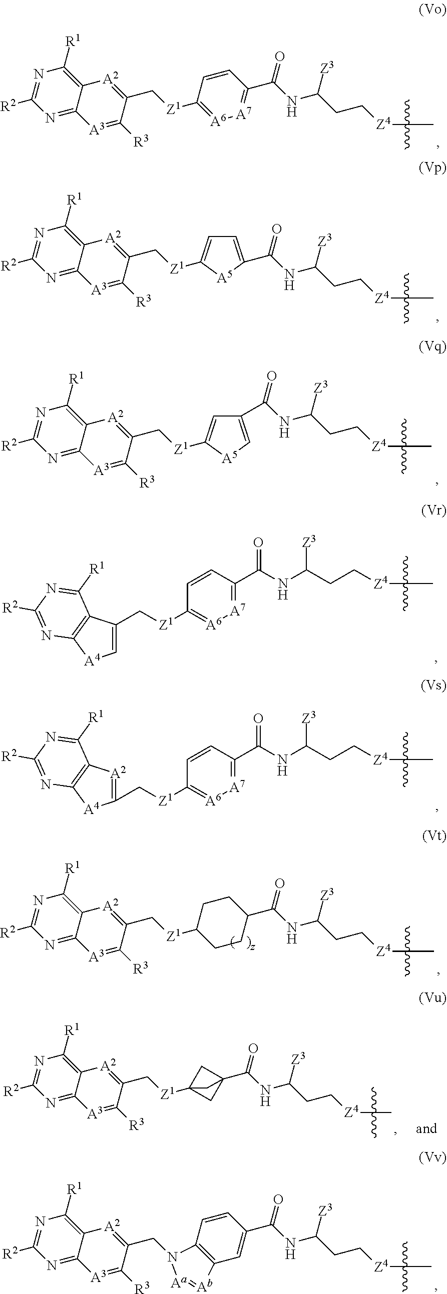

wherein:
A⁵ is selected from NR²¹, S, O, C(R²¹)₂;
A⁶ and A⁷ are each independently selected from N, and, CR²¹;
z is 0 to 3;
[symbol] is a single bond or a double bond;
wherein when [symbol] is a single bond A^{a} is selected from C(R²¹)₂, and C═O, and A^{b} is selected from C(R²¹)₂, and NR²¹; and
when [symbol] is a double bond A^{a} is CR²¹; and A^{b} is selected from CR²¹ and N.

In certain embodiments, the subject compound comprises a cell surface folate receptor ligand selected from one of the following structures:

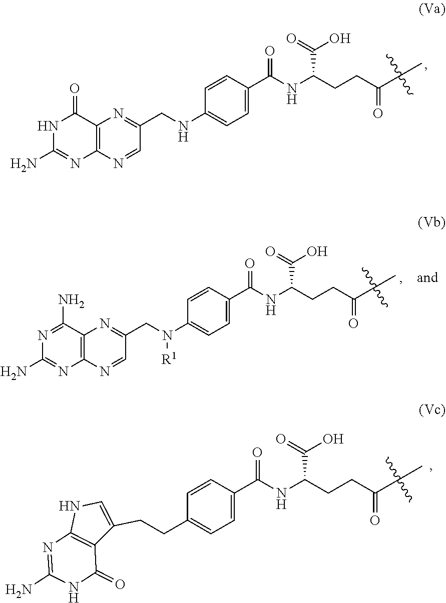

wherein R¹ is —H or —CH₃.

In certain embodiments, the subject compound comprises a cell surface folate receptor ligand is of formula (Vo) and each of R¹-R³, A²-A³, A⁶-A⁷, Z¹ and Z³-Z⁴ are as described herein above.

In certain embodiments, the subject compound comprises a cell surface folate receptor ligand is of formula (Vp) or (Vq) and each of R¹-R³, A²-A³, A⁵, Z¹ and Z³-Z⁴ are as described herein above.

In certain embodiments, the subject compound comprises a cell surface folate receptor ligand is of formula (Vr) or (Vs) and each of R¹-R², A², A⁴, A⁶-A⁷, Z¹ and Z³-Z⁴ are as described herein above.

In certain embodiments, the subject compound comprises a cell surface folate receptor ligand is of formula (Vt) and each of R¹-R³, A²-A³, z, Z¹ and Z³-Z⁴ are as described herein above.

In certain embodiments, the subject compound comprises a cell surface folate receptor ligand is of formula (Vu) and each of R¹-R³, A²-A³, Z¹ and Z³-Z⁴ are as described herein above.

In certain embodiments, the subject compound comprises a cell surface folate receptor ligand is of formula (Vv) and each of R¹-R³, A²-A³, A^{a}-A^{b}, and Z³-Z⁴ are as described herein above.

In certain embodiments, the subject compound comprises a cell surface folate receptor ligand which can be utilized in the preparation of compounds of this disclosure are shown in tables 1-2.

TABLE 1
Exemplary cell surface folate receptor binding moieties

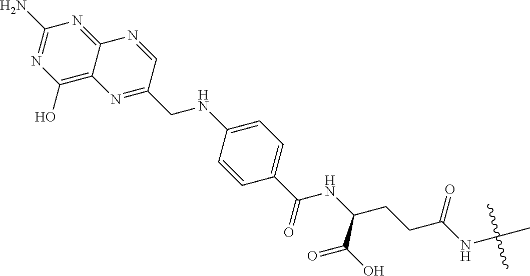 [SMILES: *NC(=O)CC[C@H](NC(=O)c1ccc(NCc2cnc3nc(N)nc(O)c3n2)cc1)C(=O)O]

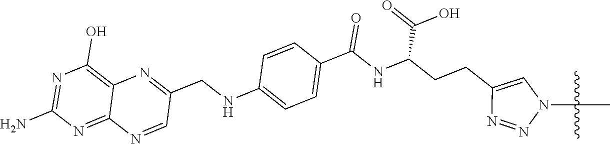 [SMILES: *n1cc(CC[C@H](NC(=O)c2ccc(NCc3cnc4nc(N)nc(O)c4n3)cc2)C(=O)O)nn1]

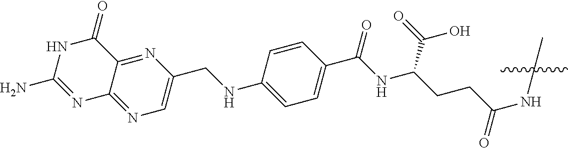 [SMILES: *NC(=O)CC[C@H](NC(=O)c1ccc(NCc2cnc3nc(N)[nH]c(=O)c3n2)cc1)C(=O)O]

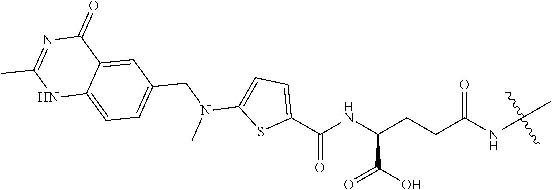 [SMILES: *NC(=O)CC[C@H](NC(=O)c1ccc(N(C)Cc2ccc3[nH]c(C)nc(=O)c3c2)s1)C(=O)O]

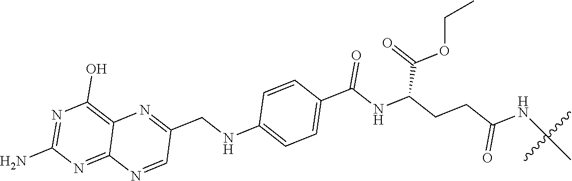 [SMILES: *NC(=O)CC[C@H](NC(=O)c1ccc(NCc2cnc3nc(N)nc(O)c3n2)cc1)C(=O)OCC]

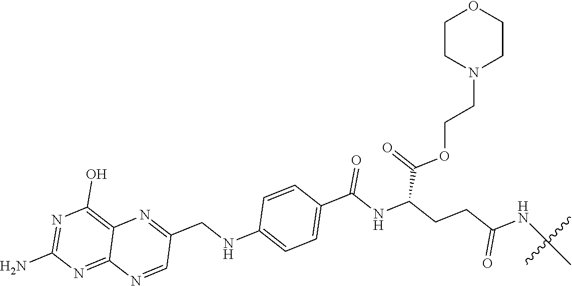 [SMILES: *NC(=O)CC[C@H](NC(=O)c1ccc(NCc2cnc3nc(N)nc(O)c3n2)cc1)C(=O)OCCN1CCOCC1]

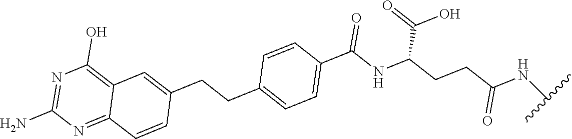 [SMILES: *NC(=O)CC[C@H](NC(=O)c1ccc(CCc2ccc3nc(N)nc(O)c3c2)cc1)C(=O)O]

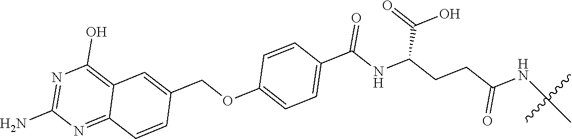 [SMILES: *NC(=O)CC[C@H](NC(=O)c1ccc(OCc2ccc3nc(N)nc(O)c3c2)cc1)C(=O)O]

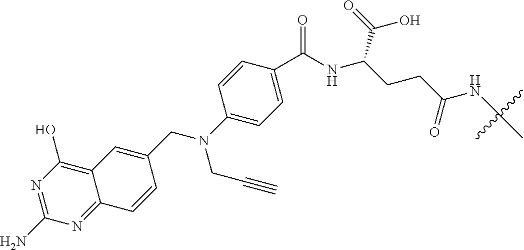 [SMILES: *NC(=O)CC[C@H](NC(=O)c1ccc(N(CC#C)Cc2ccc3nc(N)nc(O)c3c2)cc1)C(=O)O]

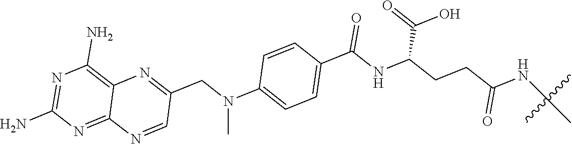 [SMILES: *NC(=O)CC[C@H](NC(=O)c1ccc(N(C)Cc2cnc3nc(N)nc(N)c3n2)cc1)C(=O)O]

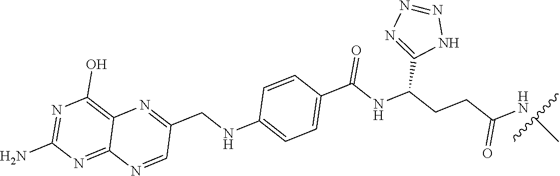

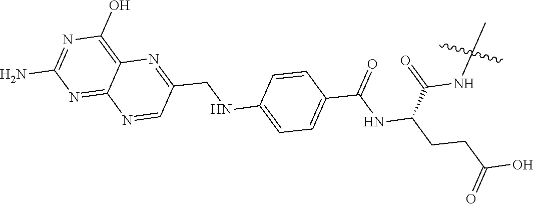 [SMILES: *NC(=O)[C@H](CCC(=O)O)NC(=O)c1ccc(NCc2cnc3nc(N)nc(O)c3n2)cc1]

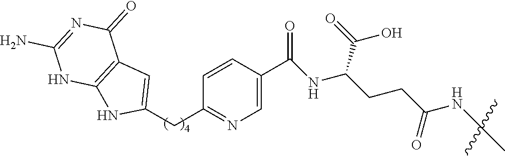

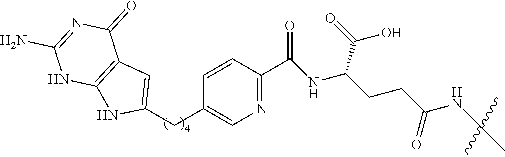

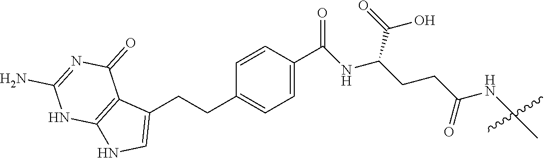 [SMILES: *NC(=O)CC[C@H](NC(=O)c1ccc(CCc2c[nH]c3[nH]c(N)nc(=O)c23)cc1)C(=O)O]

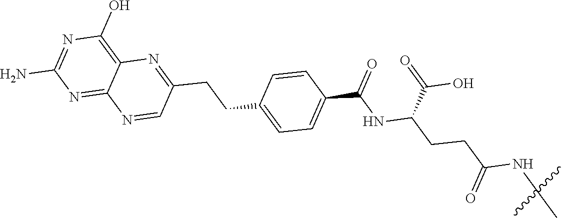

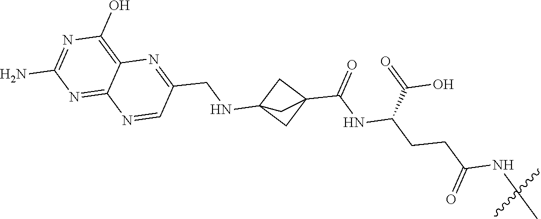 [SMILES: *NC(=O)CC[C@H](NC(=O)C12CC(NCc3cnc4nc(N)nc(O)c4n3)(C1)C2)C(=O)O]

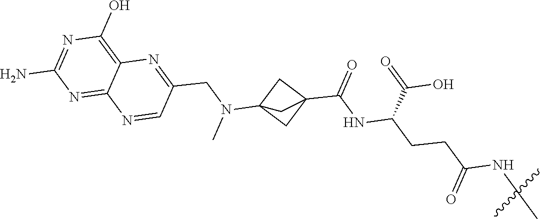 [SMILES: *NC(=O)CC[C@H](NC(=O)C12CC(N(C)Cc3cnc4nc(N)nc(O)c4n3)(C1)C2)C(=O)O]

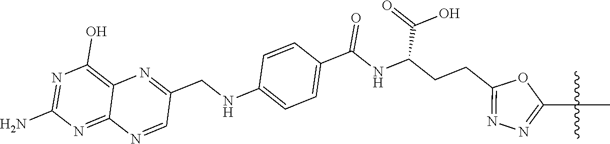 [SMILES: *c1nnc(CC[C@H](NC(=O)c2ccc(NCc3cnc4nc(N)nc(O)c4n3)cc2)C(=O)O)o1]

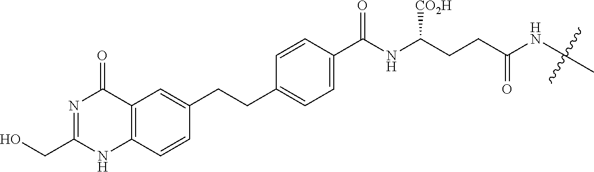 [SMILES: *NC(=O)CC[C@H](NC(=O)c1ccc(CCc2ccc3[nH]c(CO)nc(=O)c3c2)cc1)C(=O)O]

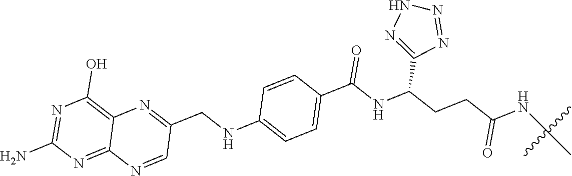 [SMILES: *NC(=O)CC[C@H](NC(=O)c1ccc(NCc2cnc3nc(N)nc(O)c3n2)cc1)c1nn[nH]n1]

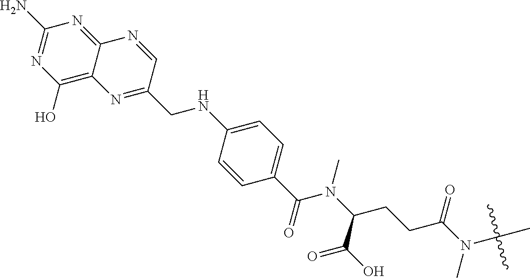 [SMILES: *N(C)C(=O)CC[C@@H](C(=O)O)N(C)C(=O)c1ccc(NCc2cnc3nc(N)nc(O)c3n2)cc1]

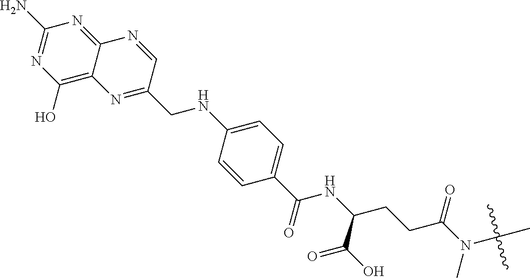 [SMILES: *N(C)C(=O)CC[C@H](NC(=O)c1ccc(NCc2cnc3nc(N)nc(O)c3n2)cc1)C(=O)O]

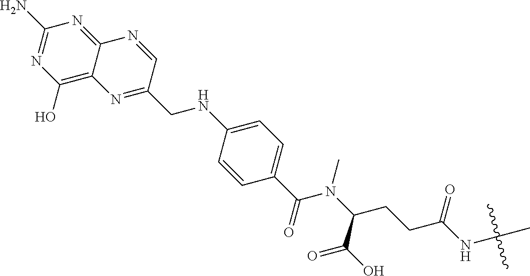 [SMILES: *NC(=O)CC[C@@H](C(=O)O)N(C)C(=O)c1ccc(NCc2cnc3nc(N)nc(O)c3n2)cc1]

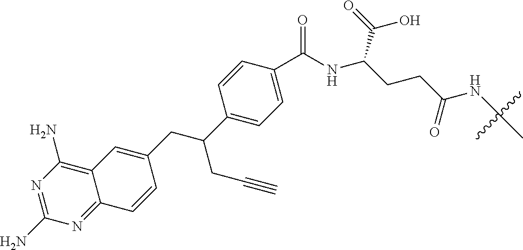 [SMILES: *NC(=O)CC[C@H](NC(=O)c1ccc(C(CC#C)Cc2ccc3nc(N)nc(N)c3c2)cc1)C(=O)O]

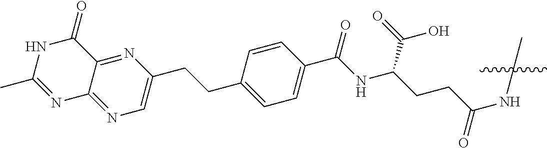 [SMILES: *NC(=O)CC[C@H](NC(=O)c1ccc(CCc2cnc3nc(C)[nH]c(=O)c3n2)cc1)C(=O)O]

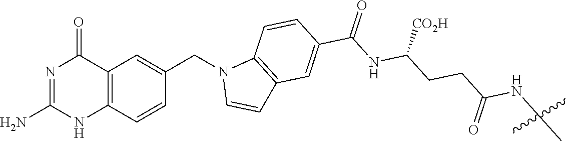 [SMILES: *NC(=O)CC[C@H](NC(=O)c1ccc2c(ccn2Cc2ccc3[nH]c(N)nc(=O)c3c2)c1)C(=O)O]

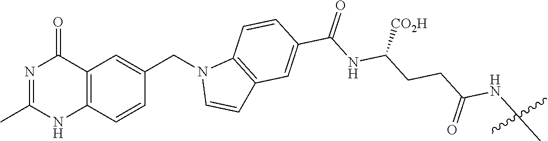 [SMILES: *NC(=O)CC[C@H](NC(=O)c1ccc2c(ccn2Cc2ccc3[nH]c(C)nc(=O)c3c2)c1)C(=O)O]

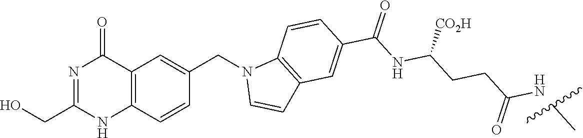

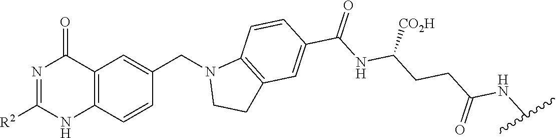

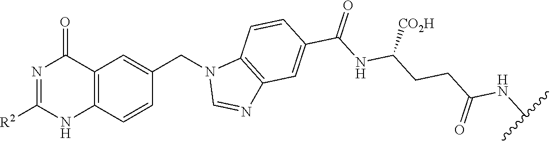

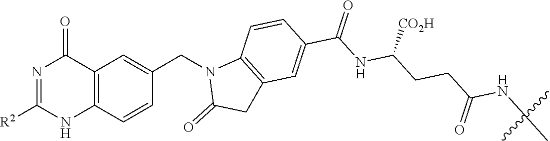

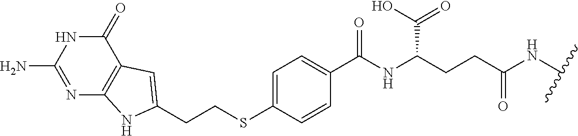 [SMILES: *NC(=O)CC[C@H](NC(=O)c1ccc(SCCc2cc3c(=O)[nH]c(N)nc3[nH]2)cc1)C(=O)O]

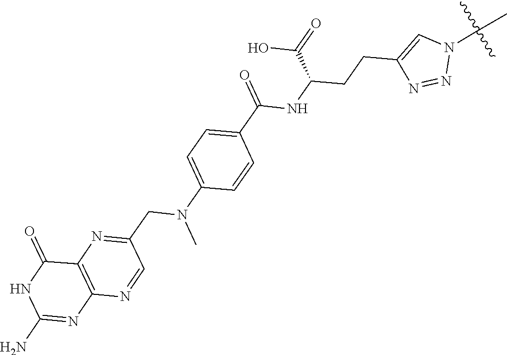 [SMILES: *n1cc(CC[C@H](NC(=O)c2ccc(N(C)Cc3cnc4nc(N)[nH]c(=O)c4n3)cc2)C(=O)O)nn1]

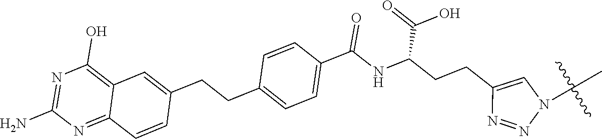 [SMILES: *n1cc(CC[C@H](NC(=O)c2ccc(CCc3ccc4nc(N)nc(O)c4c3)cc2)C(=O)O)nn1]

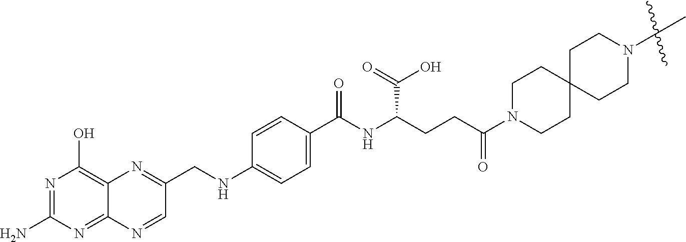 [SMILES: *N1CCC2(CC1)CCN(C(=O)CC[C@H](NC(=O)c1ccc(NCc3cnc4nc(N)nc(O)c4n3)cc1)C(=O)O)CC2]

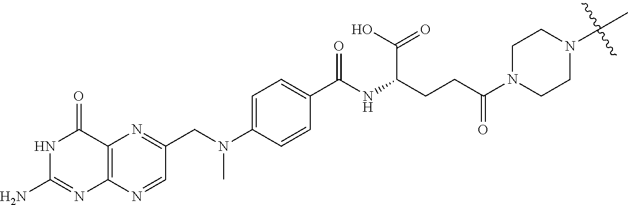 [SMILES: *N1CCN(C(=O)CC[C@H](NC(=O)c2ccc(N(C)Cc3cnc4nc(N)[nH]c(=O)c4n3)cc2)C(=O)O)CC1]

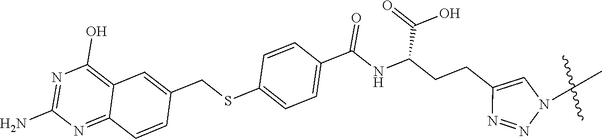 [SMILES: *n1cc(CC[C@H](NC(=O)c2ccc(SCc3ccc4nc(N)nc(O)c4c3)cc2)C(=O)O)nn1]

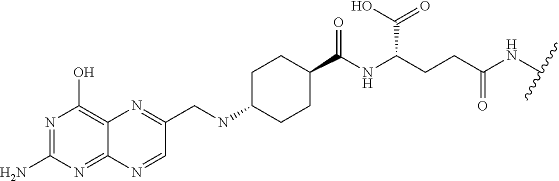 [SMILES: *NC(=O)CC[C@H](NC(=O)[C@H]1CC[C@H](NCc2cnc3nc(N)nc(O)c3n2)CC1)C(=O)O]

TABLE 2
cell surface folate receptor binding moieties

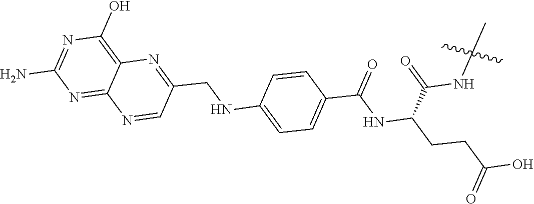 [SMILES: *NC(=O)[C@H](CCC(=O)O)NC(=O)c1ccc(NCc2cnc3nc(N)nc(O)c3n2)cc1]

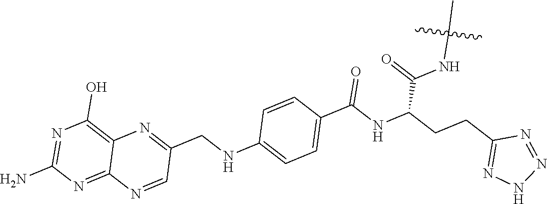 [SMILES: *NC(=O)[C@H](CCc1nn[nH]n1)NC(=O)c1ccc(NCc2cnc3nc(N)nc(O)c3n2)cc1]

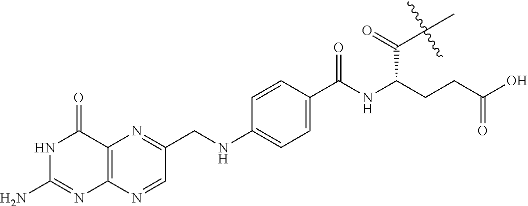 [SMILES: *C(=O)[C@H](CCC(=O)O)NC(=O)c1ccc(NCc2cnc3nc(N)[nH]c(=O)c3n2)cc1]

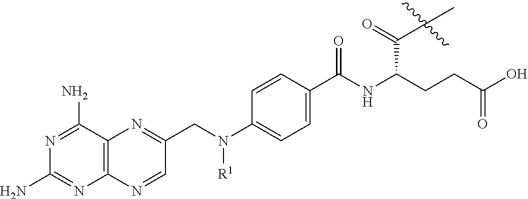

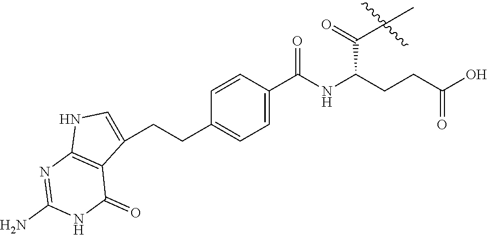 [SMILES: *C(=O)[C@H](CCC(=O)O)NC(=O)c1ccc(CCc2c[nH]c3nc(N)[nH]c(=O)c23)cc1]

In Tables 1 or 2, the [symbol] can represent the point of attachment to -L-Y.

In certain embodiments of the compound of formula (I), (IIIA), or (IIIB), n is 1. In certain cases, n is at least 2. In certain other cases, n is 2 to 20, such as 2 to 15, 2 to 10, 2 to 8, 2 to 6, or 2 to 4. In certain cases, n is 2 to 6. In certain other cases, n is 2 or 3.

Example compounds of formula (I), (IIIA) and (IIIB) are shown in tables 5-9.

### 5.1.1. Linkers

The terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest (Y), e.g., a biomolecule (e.g., an antibody) and the other for conjugation to a moiety (noted as X) that binds to a cell surface folate receptor. For example, the moiety may be folic acid or an analog of a folic acid or folate moiety. When Y is a polypeptide, the polypeptide conjugation reactive terminus of the linker is in some cases a site that is capable of conjugation to the polypeptide through a cysteine thiol or lysine amine group on the polypeptide, and so is can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In certain embodiments, L is between about 10 Å and about 20 Å in length. In certain embodiments, L is between about 15 Å and about 20 Å in length. In certain embodiments, L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to a cell surface folate receptor binding moiety (X), optionally substituted heteroarylene linked to X, optionally substituted heterocyclene linked to X, or optionally substituted cycloalkylene linked to X. In certain embodiments, L is a linker between about 10 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 400 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 200 Å, which comprises an optionally substituted arylene linked to X, optionally substituted heteroarylene linked to X, optionally substituted heterocyclene linked to X, or optionally substituted cycloalkylene linked to X.

In certain embodiments, L separates cell surface folate receptor binding moiety (Y) and Y (or Z) by a backbone comprising at least 10 consecutive atoms. In certain cases, the backbone is at least 12 consecutive atoms. In certain cases, the backbone is at least 14 consecutive atoms. In certain cases, the backbone is at least 16 consecutive atoms. In certain cases, the backbone is at least 18 consecutive atoms. In certain cases, the backbone is at least 20 consecutive atoms. In certain cases, the backbone is at least 22 consecutive atoms. In certain cases, the backbone is at least 24 consecutive atoms. In certain cases, the backbone is at least 26 consecutive atoms. In certain cases, the backbone is at least 28 consecutive atoms. In certain cases, the backbone is at least 30 consecutive atoms. In certain cases, the backbone is at least 32 consecutive atoms. In certain cases, the backbone is at least 34 consecutive atoms. In certain cases, the backbone is at least 36 consecutive atoms. In certain cases, the backbone is at least 38 consecutive atoms. In certain cases, the backbone is at least 40 consecutive atoms. In certain cases, the backbone is up to 50 consecutive atoms. In certain cases, the backbone is up to 60 consecutive atoms. In certain cases, the backbone is up to 70 consecutive atoms. In certain cases, the backbone is up to 80 consecutive atoms. In certain cases, the backbone is up to 90 consecutive atoms. In certain cases, the backbone is up to 100 consecutive atoms.

In certain embodiments, linker L separates cell surface folate receptor binding moiety (X) and Y (or Z) by a chain of 4 to 500 consecutive atoms. In certain embodiments, linker L separates X and Y (or Z) by a chain of 4 to 50 consecutive atoms. In certain embodiments, linker L separates X and Y (or Z) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z) by a chain of 6 to 50 consecutive atoms. In certain embodiments, linker L separates X and Y (or Z) by a chain of 11 to 50 consecutive atoms. In certain embodiments, linker L separates X and Y (or Z) by a chain of 16 to 50 consecutive atoms. In certain embodiments, linker L separates X and Y (or Z) by a chain of 21 to 50 consecutive atoms. In certain embodiments, linker L separates X and Y (or Z) by a chain of 26 to 50 consecutive atoms. In certain embodiments, linker L separates X and Y (or Z) by a chain of 31 to 50 consecutive atoms. In certain embodiments, linker L separates X and Y (or Z) by a chain of 36 to 50 consecutive atoms. In certain embodiments, linker L separates X and Y (or Z) by a chain of 41 to 50 consecutive atoms. In certain embodiments, linker L separates X and Y (or Z) by a chain of 46 to 50 consecutive atoms.

In certain embodiments, linker L separates X and Y (or Z) by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L separates X and Y (or Z) by a chain of 50 or 55 consecutive atoms, by a chain of 56 to 60 consecutive atoms, by a chain of 61 to 65 consecutive atoms, by a chain of 66 to 70 consecutive atoms, by a chain of 71 to 75 consecutive atoms, by a chain of 76 to 80 consecutive atoms, by a chain of 81 to 85 consecutive atoms, by a chain of 86 to 90 consecutive atoms, by a chain of 91 to 95 consecutive atoms, or by a chain of 96 to 100 consecutive atoms.

In certain embodiments, linker L is a chain of 5 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 7 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 10 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 15 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

In certain embodiments, linker L is a chain of 5 to 500 consecutive atoms separating X and Y (or Z) and which comprises an optionally substituted arylene linked to X or optionally substituted heteroarylene linked to X. In certain embodiments, linker L is a chain of 7 to 500 consecutive atoms separating X and Y (or Z) and which comprises an optionally substituted arylene linked to X or optionally substituted heteroarylene linked to X. In certain embodiments, linker L is a chain of 10 to 500 consecutive atoms separating X and Y (or Z) and which comprises an optionally substituted arylene linked to X or optionally substituted heteroarylene linked to X. In certain embodiments, linker L is a chain of 15 to 400 consecutive atoms separating X and Y (or Z) and which comprises an optionally substituted arylene linked to X or optionally substituted heteroarylene linked to X.

In certain embodiments, linker L is a chain of 5 to 500 consecutive atoms separating X and Y (or Z) and which comprises an optionally substituted phenylene linked to X. In certain embodiments, linker L is a chain of 7 to 500 consecutive atoms separating X and Y (or Z) and which comprises an optionally substituted phenylene linked to X. In certain embodiments, linker L is a chain of 10 to 500 consecutive atoms separating X and Y (or Z) and which comprises an optionally substituted phenylene linked to X. In certain embodiments, linker L is a chain of 15 to 400 consecutive atoms separating X and Y (or Z) and which comprises an optionally phenylene linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

It is understood that the linker may be considered as connecting directly to a Z⁴ group of a folate binding moiety (X) (e.g., as described herein). In some embodiments of formula (I), the linker may be considered as connecting directly to the Z³ group. Alternatively, the —Z²CH(-T³-Z³)T⁴Z⁴-group of formula (I) (e.g., as described herein) can be considered part of a linking moiety that connects Z⁴ to Y. The disclosure is meant to include all such configurations of folate binding moiety (X) and linker (L).

In certain embodiments of the subject compounds, L comprises one or more linking moieties independently selected from —C₁₋₆-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, —NH—, and —NMe-, wherein p is 1 to 10.

In certain embodiments of the subject compounds, L comprises one or more —C₁₋₆-alkylene-linking moieties. In certain cases, L comprises one or more —NHCO—C₁₋₆-alkylene-linking moieties. In certain cases, L comprises one or more —CONH—C₁₋₆-alkylene-linking moieties. In certain cases, L comprises one or more —NH C₁₋₆-alkylene-linking moieties. In certain cases, L comprises one or more —NHCONH—C₁₋₆-alkylene-linking moieties. In certain cases, L comprises one or more —NHCSNH—C₁₋₆-alkylene-linking moieties. In certain cases, L comprises one or more —C₁₋₆-alkylene-NHCO— linking moieties. In certain cases, L comprises one or more —C₁₋₆-alkylene-CONH-linking moieties. In certain cases, L comprises one or more —C₁₋₆-alkylene-NH— linking moieties. In certain cases, L comprises one or more —C₁₋₆-alkylene-NHCONH— linking moieties. In certain cases, L comprises one or more —C₁₋₆-alkylene-NHCSNH— linking moieties. In certain cases, L comprises one or more —O(CH₂)ₚ— linking moieties. In certain cases, L comprises one or more —(OCH₂CH₂)ₚ— linking moieties. In certain cases, L comprises one or more —NHCO— linking moieties. In certain cases, L comprises one or more —CONH— linking moieties. In certain cases, L comprises one or more —NHSO₂— linking moieties. In certain cases, L comprises one or more —SO₂NH— linking moieties. In certain cases, L comprises one or more —CO— linking moieties. In certain cases, L comprises one or more —SO₂— linking moieties. In certain cases, L comprises one or more —O-linking moieties. In certain cases, L comprises one or more —S— linking moieties. In certain cases, L comprises one or more pyrrolidine-2,5-dione linking moieties. In certain cases, L comprises one or more —NH— linking moieties. In certain cases, L comprises one or more —Nme- linking moieties.

In certain embodiments of the subject compounds, L comprises repeating ethylene glycol moieties (e.g., —CH₂CH₂O—or —OCH₂CH₂—). In certain case, L comprises I to 20 ethylene glycol moieties. In certain cases, L comprise 2 to 18 ethylene glycol moieties. In certain cases, L comprise 2 to 16 ethylene glycol moieties. In certain cases, L comprises 2 to 14 ethylene glycol moieties. In certain cases, L comprises 2 to 12 ethylene glycol moieties. In certain cases, L comprises 2 to 10 ethylene glycol moieties. In certain cases, L comprises 2 to 8 ethylene glycol moieties. In certain cases, L comprises 2 to 8 ethylene glycol moieties. In certain cases, L comprises 2 to 6 ethylene glycol moieties.

In certain embodiments, L is of formula (IV):

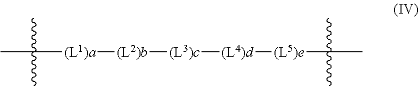

wherein
each L¹ to L⁵ is independently a linking moiety which together provide a linear or branched linker between Z⁴ and Y;
a is 1 or 2;
b, c, d, and e are each independently 0, 1, or 2.

In certain embodiments of formula (IV), -(L¹)ₐ- comprises an optionally substituted alkyl or ethylene glycol linking moiety. In certain cases, L¹ comprises an optionally substituted —C₁₋₆-alkylene-. In certain cases, L¹ comprises an ethylene glycol linking moiety.

In certain embodiments of formula (IV), L¹ is independently selected from: —C₁₋₆-alkylene-, —(CH₂CH₂O)ₜ—, —C₁₋₆-alkylene-NR⁴CO—, —C₁₋₆-alkyleneCONH—, or OCH₂, wherein t is 1 to 20; and R⁴ is independently selected from H, and optionally substituted (C₁-C₆)alkyl. In certain cases, L¹ is —C₁₋₆-alkylene-, such as —C₁₋₃-alkylene-. In certain cases, L¹ is —(CH₂CH₂O)ₜ—, where t is 1 to 20, such as 1 to 15, 1 to 10, 1 to 8, 1 to 6, or 1 to 4. In certain cases, L¹ is —C₁₋₆-alkylene-NR⁴CO—. In certain cases, L¹ is —C₁₋₆-alkyleneCONH—. In certain cases, L¹ is or OCH₂.

In certain embodiments of formula (IV), L² is independently selected from: —NR⁴CO—C₁₋₆-alkylene-, —CONR⁴—C₁₋₆-alkylene,

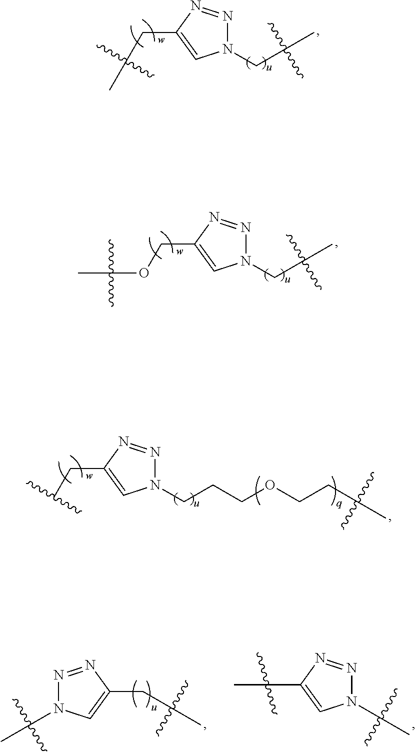

—OCH₂—, and —(OCH₂CH₂)_{q}—, wherein q is 1 to 10, u is 0 to 10, w is 1 to 10, and R⁴ is independently selected from H, and optionally substituted (C₁-C₆)alkyl. In certain cases, L² is —NR⁴CO—C₁₋₆-alkylene-. In certain cases, L² is —CONR⁴—C₁₋₆-alkylene.

In certain cases, L² is

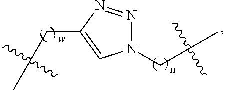

where w is 1 and u is 0 or 1.

In certain cases, L² is

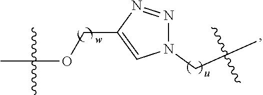

where w is 1 and u is 0 or 1.

In certain cases, L² is

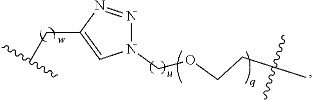

where w is 1, u is 0 or 1, and q is 1.

In certain cases, L² is

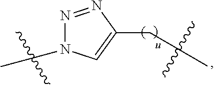

where u is 0 or 1.

In certain cases, L² is

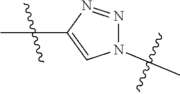 [SMILES: *c1cn(*)nn1]

In certain embodiments, L² is —OCH₂—. In certain other embodiments, L² is (OCH₂CH₂)_{q}—, and q is 1 to 10, 1 to 9, 1 to 8, 1 to 7, 1 to 6, 1 to 5, 1 to 4, 1 to 3 or 1 to 2. In certain cases, q is 2 to 8, such as 2 to 6, 4 to 6, or 2 to 4.

In certain embodiments of formula (IV), L⁴ is absent or independently selected from —C₁₋₆-alkylene-, —(CH₂CH₂O)ₜ—, —C₁₋₆-alkylene-NHCO—, —C₁₋₆-alkyleneCONH—, or OCH₂, wherein t is 1 to 20. In certain cases, L⁴ is absent. In certain cases, L⁴ is —C₁₋₆-alkylene-. In certain cases, L⁴ is —(CH₂CH₂O)ₜ—, where t is 1 to 20, such as 1 to 15, 1 to 12, 1 to 10, 1 to 8, 1 to 6, 1 to 4 or 1 to 3. In certain cases, L⁴ is —C₁₋₆-alkylene-NHCO—. In certain cases, L⁴ is —C₁₋₆-alkyleneCONH—. In certain cases, L⁴ is OCH₂,

In some embodiments of formula (IV), each L³ is a linear or branched linking moiety. In certain cases, L³ is a linear linking moiety. In certain cases, L³ is —OCH₂CH₂—.

In certain embodiments of the subject compounds, n is 2 or more, at least one L³ is present and is a branched linking moiety.

Accordingly, in some embodiments of formula (IV), L³ is a branched linking moiety, e.g., a trivalent linking moiety. For example, an L³ linking moiety can be of the one of the following general formula:

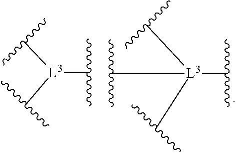

In some embodiments of formula (IV), the branched linking moiety can be of higher valency and be described by one of the one of the following general formula:

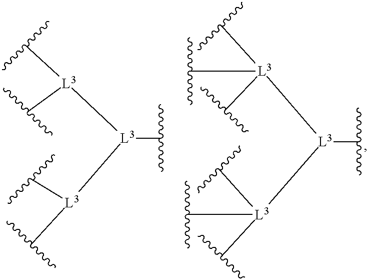

etc.
where any two L³ groups can be directed linked or connected via optional linear linking moieties (e.g., as described herein).

In some embodiments of formula (IV), the branched linking moiety can include one, two or more L³ linking moieties, each being trivalent moieties, which when linked together can provide for multiple branching points for covalent attachment of the ligands and be described by one of the one of the following general formula:

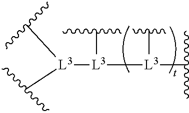

where t is 0 to 500, such as 0 to 100, 0 to 20, or 0 to 10.

In some embodiments, the branched linking moiety (e.g., L³) comprises one or more of: an amino acid residue (e.g., Asp, Lys, Orn, Glu), N-substituted amido (—N(—)C(═O)—), tertiary amino, polyol (e.g., O-substituted glycerol), and the like.

In some embodiments of formula (IV), one or more L³ is a branching moiety selected from

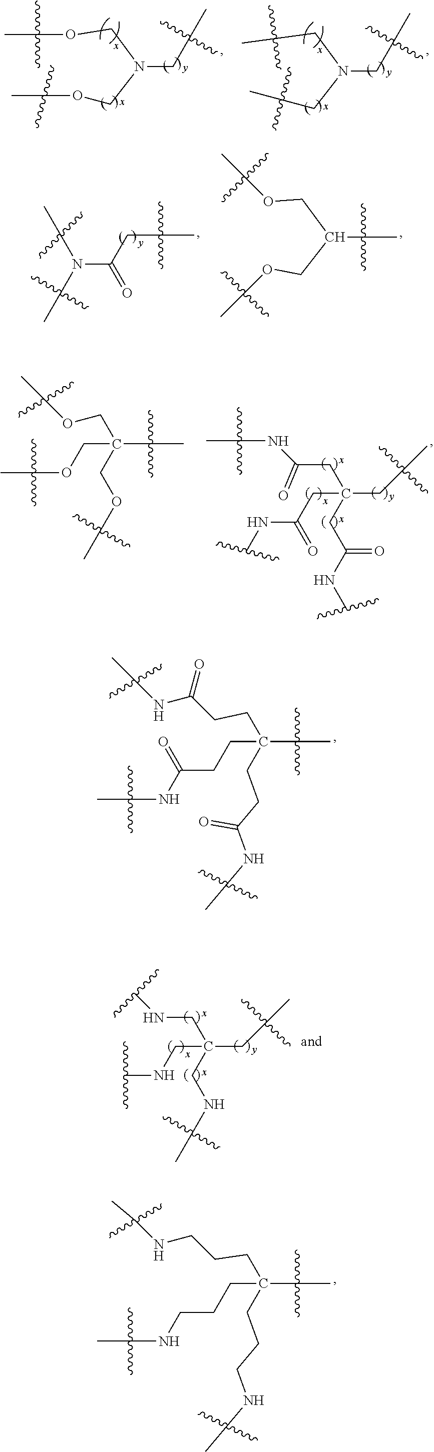

wherein each x and y are each independently 1 to 10, such as 1-6, 1-3, e.g., 1 or 2. In some cases, each x is 1, 2 or 3, e.g., 2.

In some embodiments of formula (IV), one or more L⁵ is independently —CH₂O—; —(CH₂O)ₜ—, —NR⁴CO—, —C₁₋₆-alkylene-,

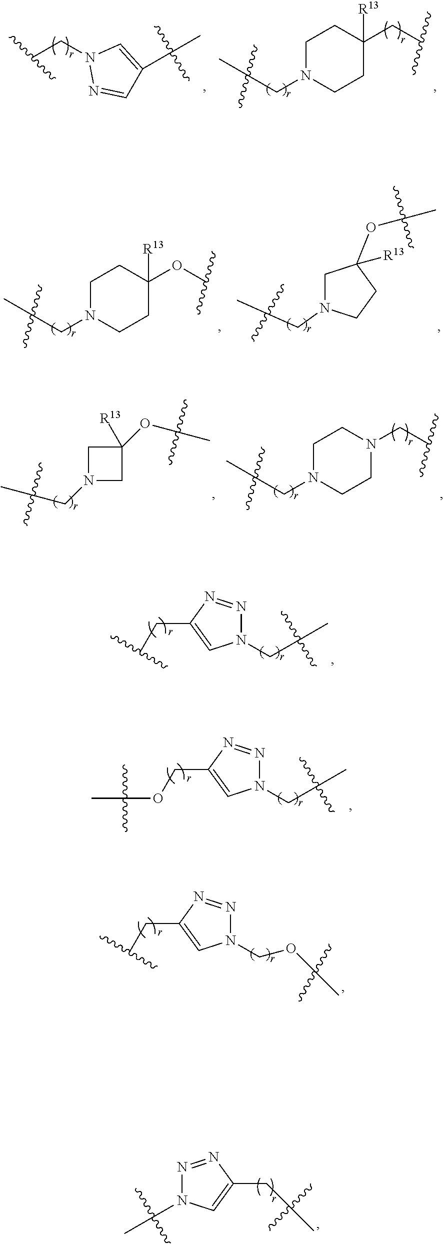

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; each r independently 0 to 20, and any of the L⁵ moieties are optionally further substituted.

In certain cases, L⁵ is —CH₂O—. In certain cases, L⁵ is —(CH₂CH₂O)ₜ—, where t is 1 to 20, such as 1-15, 1-12, 1-10, 1-8, 1-6, or 1 to 4. In certain cases, L⁵ is —NR⁴CO—, where R⁴ is H, or optionally substituted (C₁-C₆)alkyl. In certain cases, L⁵ is —C₁₋₆-alkylene-.

In certain cases, L⁵ is

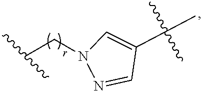

where r is 0 to 20, such as 0 to 15, 0 to 10, 0 to 8, or 0 to 5.

In certain cases, L⁵ is

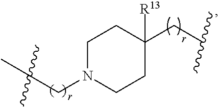

where each r is independently 0 to 20, such as 0 to 15, 0 to 10, 0 to 8, or 0 to 5 and R¹³ is H, or optionally substituted (C₁-C₆)alkyl.

In certain cases, L⁵ is

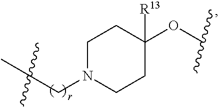

where r is 0 to 20, such as 0 to 15, 0 to 10, 0 to 8, or 0 to 5 and R¹³ is H, or optionally substituted (C₁-C₆)alkyl.

In certain cases, L⁵ is

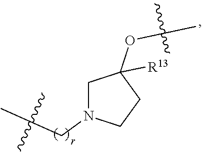

where r is 0 to 20, such as 0 to 15, 0 to 10, 0 to 8, or 0 to 5, and R¹³ is H, or optionally substituted (C₁-C₆)alkyl.

In certain cases, L⁵ is

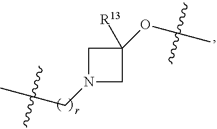

where r is 0 to 20, such as 0 to 15, 0 to 10, 0 to 8, or 0 to 5, and R¹³ is H, or optionally substituted (C₁-C₆)alkyl.

In certain cases, L⁵ is

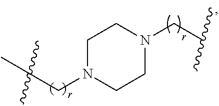

where each r is independently 0 to 20, such as 0 to 15, 0 to 10, 0 to 8, or 0 to 5.

In certain cases, L⁵ is

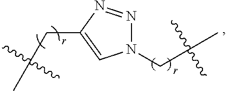

where each r is independently 0 to 20, such as 0 to 15, 0 to 10, 0 to 8, or 0 to 5.

In certain cases, L⁵ is

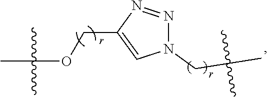

where each r is independently 0 to 20, such as 0 to 15, 0 to 10, 0 to 8, or 0 to 5.

In certain cases, L⁵ is

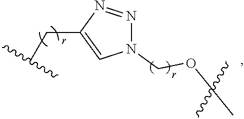

where each r is independently 0 to 20, such as 0 to 15, 0 to 10, 0 to 8, or 0 to 5.

In certain cases, L⁵ is

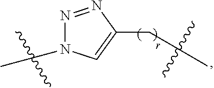

where r is 0 to 20, such as 0 to 15, 0 to 10, 0 to 8, or 0 to 5.

In certain embodiments of formula (IV), a is 1. In certain cases, at least one of b, c, d, and e is not 0. In certain cases, b is 1 or 2. In certain cases, c is 1 or 2. In certain cases, e is 1 or 2. In certain cases, b, d and e are independently 1 or 2. In certain cases, a, b, d, and e are each 1, and c is 0.

In certain embodiments of formula (IV), the linker comprises one or more of: an amino acid residue (e.g., Asp, Lys, Orn, Glu), an amino acid analogue, N-substituted amido (—N(—)C(═O)—), tertiary amino, polyol (e.g., O-substituted glycerol), and the like. Analogs of an amino acid, include but not limited to, unnatural amino acids, as well as other modifications known in the art. The amino acid includes L-amino acids, D-amino acids, or both, and may contain any of a variety of amino acid modifications or analogs known in the art.

In some embodiments of formula (IV), L comprises one or more of the following units:

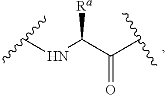

where Ra is (C₁-C₆)alkyl or substituted (C₁-C₆)alkyl, e.g., a (C₁-C₆)alkyl optionally substituted with amine, a tertiary amine, optionally substituted alkoxy, optionally substituted carboxyl, optionally substituted aryl, or optionally substituted heteroaryl.

In some embodiments of formula (IV), L has or comprises the following structure:

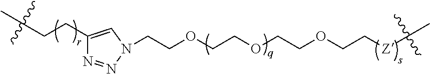

wherein:
r is 0 to 10;
q is 0 to 20 (e.g., 1, 2, 3, 4, 5, 6, 7, 8, 9, or 10);
s is 0 or 1;
Z′ is CO, NHCO, CONH or NH.

In some embodiments of formula (IV), L has or comprises the following structure:

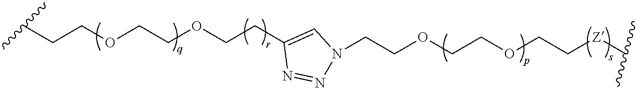

wherein:
r is 0 to 10;
p and q are 0 to 20;
s is 0 or 1; and
Z′ is CO, NHCO, CONH or NH.

In some embodiments of formula (IV), L has or comprises one of the following structures:

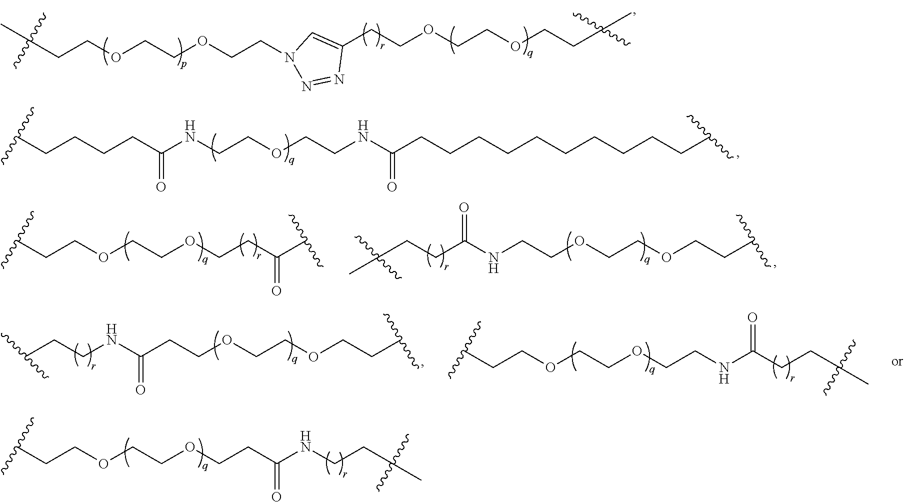

wherein r is 0 to 10, and p and q are independently 0 to 20.

Table 3 shows a variety of example linkers or linking moieties that find use in the compounds described herein. In some embodiments of formula (I), (IIIA) or (IIIB), the compound includes any one of the linkers or linking moieties set forth in Table 3.

TABLE 3
Example linkers of formula (IV)
Compound structure

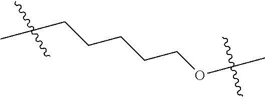 [SMILES: *CCCCCO*]

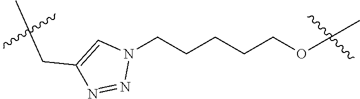 [SMILES: *Cc1cn(CCCCCO*)nn1]

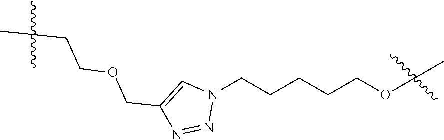 [SMILES: *CCOCc1cn(CCCCCO*)nn1]

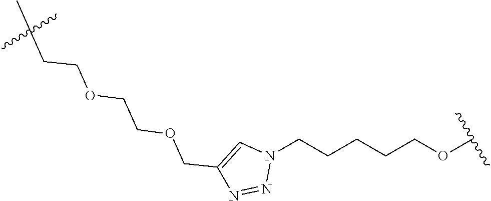 [SMILES: *CCOCCOCc1cn(CCCCCO*)nn1]

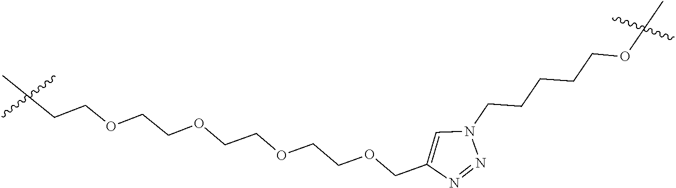 [SMILES: *CCOCCOCCOCCOCc1cn(CCCCCO*)nn1]

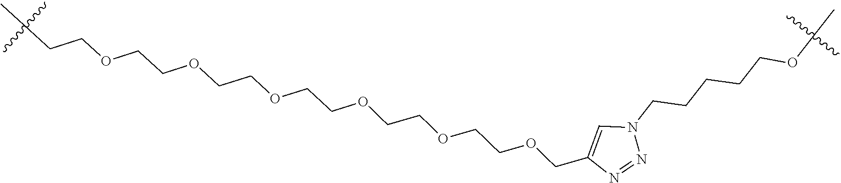 [SMILES: *CCOCCOCCOCCOCCOCCOCc1cn(CCCCCO*)nn1]

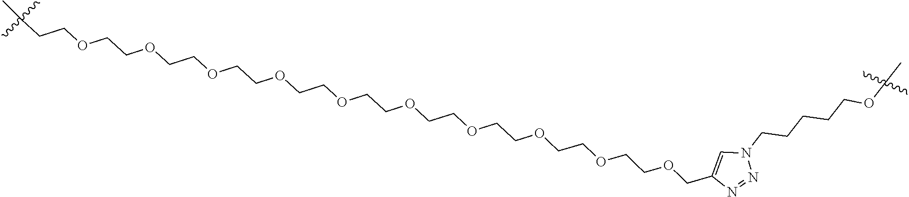 [SMILES: *CCOCCOCCOCCOCCOCCOCCOCCOCCOCCOCc1cn(CCCCCO*)nn1]

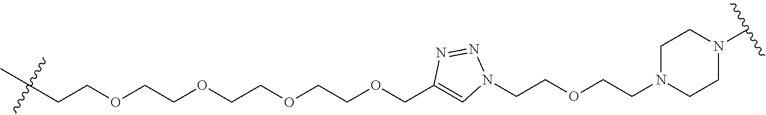 [SMILES: *CCOCCOCCOCCOCc1cn(CCOCCN2CCN(*)CC2)nn1]

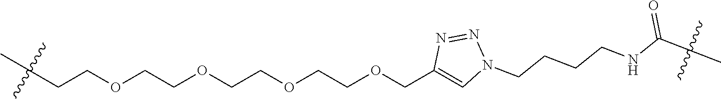 [SMILES: *CCOCCOCCOCCOCc1cn(CCCCNC(*)=O)nn1]

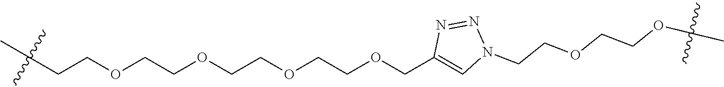 [SMILES: *CCOCCOCCOCCOCc1cn(CCOCCO*)nn1]

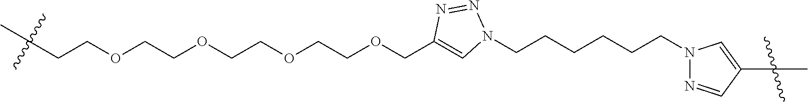 [SMILES: *CCOCCOCCOCCOCc1cn(CCCCCCn2cc(*)cn2)nn1]

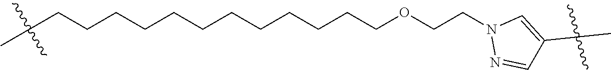 [SMILES: *CCCCCCCCCCCCOCCn1cc(*)cn1]

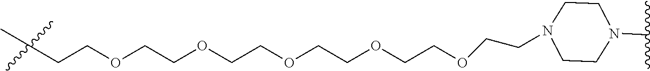 [SMILES: *CCOCCOCCOCCOCCOCCN1CCN(*)CC1]

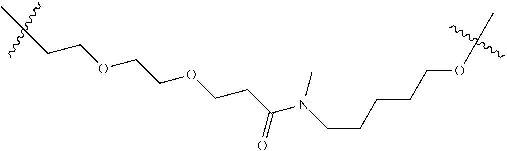 [SMILES: *CCOCCOCCC(=O)N(C)CCCCCO*]

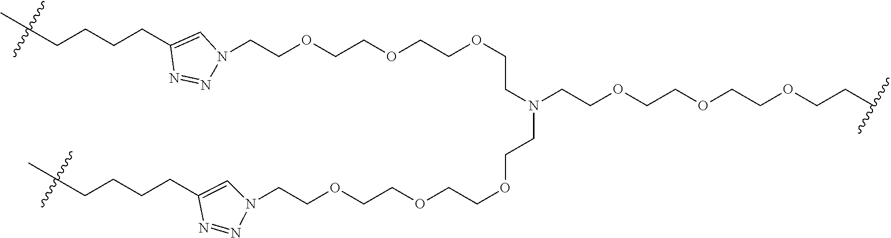 [SMILES: *CCCCc1cn(CCOCCOCCOCCN(CCOCCOCCOCC*)CCOCCOCCOCCn2cc(CCCC*)nn2)nn1]

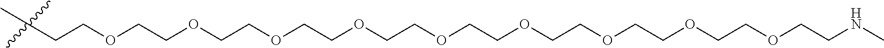 [SMILES: *CCOCCOCCOCCOCCOCCOCCOCCOCCOCCNC]

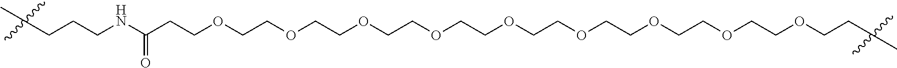 [SMILES: *CCCNC(=O)CCOCCOCCOCCOCCOCCOCCOCCOCCOCC*]

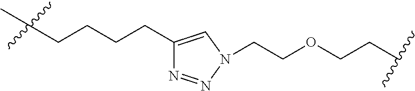 [SMILES: *CCCCc1cn(CCOCC*)nn1]

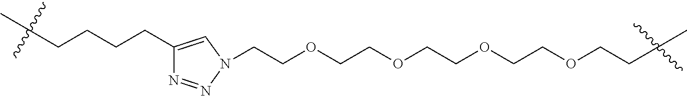 [SMILES: *CCCCc1cn(CCOCCOCCOCCOCC*)nn1]

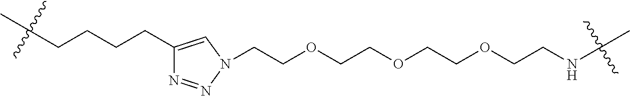 [SMILES: *CCCCc1cn(CCOCCOCCOCCN*)nn1]

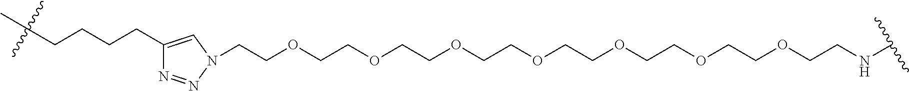 [SMILES: *CCCCc1cn(CCOCCOCCOCCOCCOCCOCCOCCN*)nn1]

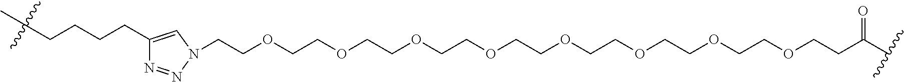 [SMILES: *CCCCc1cn(CCOCCOCCOCCOCCOCCOCCOCCOCCC(*)=O)nn1]

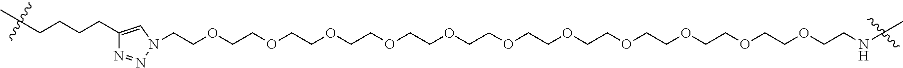 [SMILES: *CCCCc1cn(CCOCCOCCOCCOCCOCCOCCOCCOCCOCCOCCOCCN*)nn1]

In Table 3, the [symbol] can represent the point of attachment to X (on the left hand terminus) and Y (on the right hand terminus). It is understood that in any of the linking groups shown in Table 3, additional terminal groups (e.g., deriving from a functional group linkage) can be incorporated, such as —NH—, —CO—, —O—, —S—S—, —S—, —CONH—, SO2NH—, —NHCO—, NHSO2-, —COO—, etc.

### 5.1.2. Moiety of Interest (Y)

As summarized above, the compounds of this disclosure generally include a linked moiety of interest Y. In some embodiments, the moiety of interest Y is a chemoselective ligation group or a precursor thereof, and the compound can find use in the preparation of a variety of conjugates via conjugation of the chemoselective ligation group to a compatible reactive group of another moiety or molecule of interest, e.g., as described herein.

In some embodiments, the moiety of interest Y to which the folate receptor ligand is linked is itself a target molecule whose delivery to the interior of a target cell is desired. In some embodiments, Y is a target molecule that is a diagnostic agent. In some embodiments, Y is a target molecule that is a therapeutic agent.

In certain other embodiments, the moiety of interest Y to which the folate receptor ligand is linked is a moiety that specifically binds to an extracellular target protein. In certain cases, the target protein is a membrane bound protein. In certain cases, the target protein is a soluble extracellular protein. In such cases, the compounds of this disclosure bind to the extracellular target protein and can provide for folate receptor mediated internalization into the cell. The extracellular target protein of interest can be sequestered and/or degraded in the cell's lysosome.

In certain embodiments, the compound is a conjugate where Y is selected from a small molecule, peptide, protein, a dye, a fluorophore, a monosaccharide, a polysaccharide (e.g., disaccharide, or trisaccharide), lipid, enzyme, enzyme substrate, and a chemoselective ligation group, or precursor thereof.

In certain embodiments of the subject compounds, Y is a target-binding small molecule. In certain cases, Y is a small molecule inhibitor or ligand of the target protein.

In certain embodiments, the target protein of interest is PCSK9. In certain embodiments, Y is a small molecule that binds to PCSK9, such as any binder recited in WO2018/057409, or WO2021072269.

In certain embodiments, the target protein is VEGF.

In certain embodiments, the target protein is TGF-beta.

In certain embodiments, the target protein is IgA. In certain embodiments, Y that binds to IgA includes a peptide binder, or a Fc-alpha receptor peptide mimetic.

In certain embodiments, the target protein is MIF.

In certain embodiments, the target protein is TNFα.

In some embodiments, Y is a target molecule that is a therapeutic agent.

In certain embodiments, Y is a chemotherapeutic agent. In certain embodiments, Y is a cytotoxic anticancer agent. Anticancer agents of interest which can be adapted for use in the subject compounds and conjugates include but are not limited to, antimitotic agent containing an epothilone moiety, antimitotic agent, e.g., maytansinoid DM4, vinca alkaloid, vinblastine, mitomycin C, paclitaxel, taxol or taxol derivative, and the like.

In some embodiments, Y is an immunotherapeutic agent. In some embodiments, Y is a cancer immunotherapeutic.

In some embodiments, Y is a toll-like receptor (TLR) ligand, e.g., a TLR agonist or antagonist. Any convenient TLR can be targeted, including but not limited to TLR3, TLR4, TLR7, TLR8, and TLR9. Any convenient TLR ligands, e.g., agonists, can be adapted for use in the compounds and conjugates of this disclosure, such as the TLR ligands described in US 20180289789. The TLR ligand can be PAMP ligand (pathogen-associated molecular patterns), an endogenous ligand, or a synthetic ligand. In some embodiments, the target TLR is TLR4. In some embodiments, Y is a lipopolysaccharide (LPS). In some embodiments, Y is a TLR ligand selected from al-acid glycoprotein (AGP), monophosphoryl lipid A (MPLA), RC-529, MDF2β, and complete Freund's adjuvant (CFA). In some embodiments, Y is a CpG oligonucleotide, e.g., a TLR9 binding oligonucleotide containing a CpG motifs.

In certain embodiments, Y is a target-binding biomolecule. In certain cases, the biomolecule is selected from peptide, protein, glycoprotein, polynucleotide, aptamer, and antibody or antibody fragment. In certain cases, Y is selected from an antibody or an antibody fragment (e.g., an antigen-binding fragment of an antibody), chimeric fusion protein, an engineered protein domain, and a D-protein binder of target protein.

### 5.1.2.1 Chemoselective Ligation Groups

In certain embodiments of formula (I), Y is a chemoselective ligation group, or a precursor thereof. A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, reductive amination, dialkyl squarate chemistry, etc.

Table 4 illustrates exemplary synthetic precursors of linker components that are used to prepare compounds of this disclosure and which have various chemoselective ligation groups. A variety of other chemical functional groups can also be incorporated as needed to prepare a desired linker.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

In some instances, chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances, Y is a precursor of the reactive functionality or function group capable of conjugation to a compatible group of a second moiety. For example, a carboxylic acid is a precursor of an active ester chemoselective ligation group.

In certain embodiments of formula (I), Y is a reactive moiety capable forming a covalent bond to a polypeptide (e.g., with an amino acid sidechain of a polypeptide having a compatible reactive group). The reactive moiety can be referred to as a chemoselective ligation group.

Example chemoselective ligation groups, and synthetic precursors thereof, which may be adapted for use in the compounds of this disclosure are shown in Table 4.

TABLE 4
Exemplary chemoselective ligation groups and precursors

| Groups | Exemplary structures |
|---|---|
| carboxylic acid or active ester | 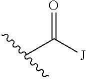 |
| | where J is selected from —OH, —Cl, —Br, —I, —F, —OH, —O—N-succinimide, |
| | —O-(4-nitrophenyl), —O-pentafluorophenyl, —O-tetrafluorophenyl, and |
| | —O—C(O)—OR^{J′}, and R^{J′} is —C_{1-C8} alkyl or -aryl, |
| | 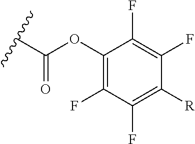 |
| | R is H or F, |
| | 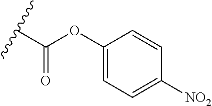 [SMILES: *C(=O)Oc1ccc([N+](=O)[O-])cc1] |
| | 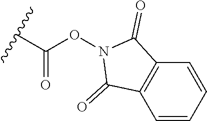 [SMILES: *C(=O)ON1C(=O)c2ccccc2C1=O] |
| | 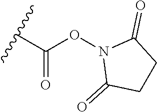 [SMILES: *C(=O)ON1C(=O)CCC1=O] |
| | 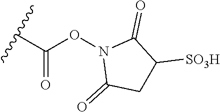 [SMILES: *C(=O)ON1C(=O)CC(S(=O)(=O)O)C1=O] |
| | 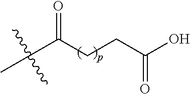 |
| | 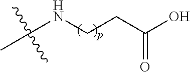 |
| | where p is 0 to 6 |
| maleimide | 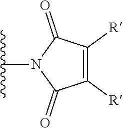 |
| | where each R′ is independently hydrogen or halogen (e.g., bromo) |
| isocyanate or | —NCS |
| isothiocyanate | —NCO |
| | 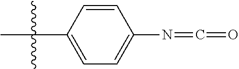 [SMILES: *c1ccc(N=C=O)cc1] |
| | 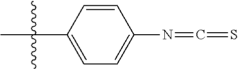 [SMILES: *c1ccc(N=C=S)cc1] |
| alkyl halide alkyl tosylate | 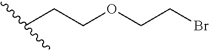 [SMILES: *CCOCCBr] |
| | 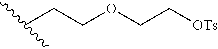 [SMILES: *CCOCCOS(=O)(=O)c1ccc(C)cc1] |
| aldehyde | 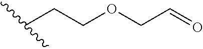 [SMILES: *CCOCC=O] |
| haloacetamide or alpha-leaving group acetamide | 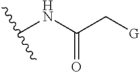 |
| | where G is selected from —F, —Cl, —Br, —I, —O-mesyl, and |
| | —O-tosyl |
| | 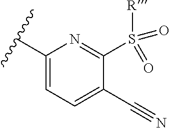 |
| | where R′′′ is alkyl |
| diazirine | 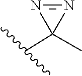 [SMILES: *C1(C)N=N1] |
| sulfonyl halide or vinyl sulfone | 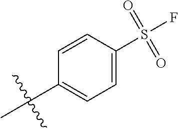 [SMILES: *c1ccc(S(=O)(=O)F)cc1] |
| | 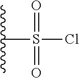 [SMILES: *S(=O)(=O)Cl] |
| | 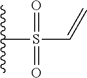 [SMILES: *S(=O)(=O)C=C] |
| hydrazide hydrazino hydroxylamino | 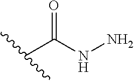 [SMILES: *C(=O)NN] |
| | 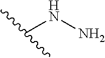 [SMILES: *NN] |
| | 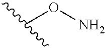 [SMILES: *ON] |
| pyridyl disulfide | 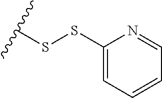 [SMILES: *SSc1ccccn1] |
| (HIPS) hydrazinyl- indolyl group, or (aza-HIPS) hydrazinyl- pyrrolo-pyridinyl group | 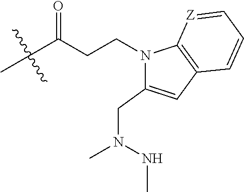 |
| | where Z is CH or N |
| alkyne or cyclooctyne | 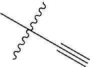 [SMILES: *C#C] |
| | 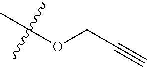 [SMILES: *OCC#C] |
| | 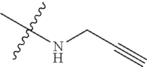 [SMILES: *NCC#C] |
| | 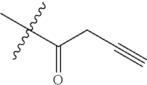 [SMILES: *C(=O)CC#C] |
| | 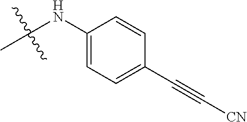 [SMILES: *Nc1ccc(C#CC#N)cc1] |
| | 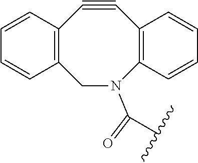 [SMILES: *C(=O)N1Cc2ccccc2C#Cc2ccccc21] |
| | 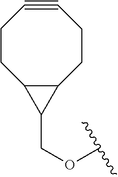 [SMILES: *OCC1C2CCC#CCCC21] |
| azide | 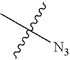 [SMILES: *N=[N+]=[N-]] |
| | 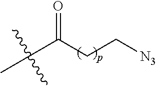 |
| | 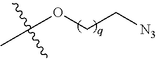 |
| | 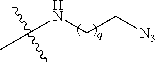 |
| | where p is 0 to 6 and where q is 1 to 6 |
| amine | 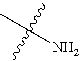 [SMILES: *N] |
| | 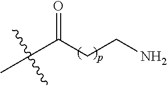 |
| | 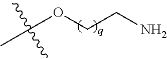 |
| | 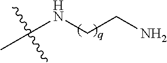 |
| | where p is 0 to 6 and where q is 1 to 6 |

In Table 4, the [symbol] can represent a point of attachment of Y to a linking moiety or a linked X moiety (e.g., FR binding moiety).

### 5.1.2.2 Polypeptide Groups

In certain embodiments, Y is a polypeptide that binds to a soluble (e.g., secreted) polypeptide of interest. In certain embodiments, for example, the polypeptide of interest is a ligand that binds a cell surface receptor and Y is a polypeptide that comprises the ligand binding portion of the cell surface receptor, for example, the extracellular domain of the cell surface receptor, e.g., a ligand-binding domain of the extracellular domain of the cell surface receptor. In certain embodiments, polypeptide of interest is a cell surface receptor and Y is a polypeptide that comprises a ligand that binds the cell surface receptor or a receptor-binding portion of the ligand.

A Y group (e.g., a polypeptide) that binds to a polypeptide of interest binds as “binding” in this context is understood by one skilled in the art. For example, Y, e.g., a polypeptide, an antibody, or a conjugate as described herein comprising such Y groups, may bind to other polypeptides, generally with lower affinity as determined by, e.g., immunoassays or other assays known in the art. In a specific embodiment, Y, or a conjugate as described herein comprising such Y groups that specifically bind to a polypeptide of interest binds to the polypeptide of interest with an affinity that is at least 2 logs, 2.5 logs, 3 logs, 4 logs or greater than the affinity when Y or the conjugate bind to another polypeptide. In another specific embodiment, Y, or a conjugate as described herein comprising such Y groups, does not specifically bind a polypeptide other than the polypeptide of interest. In a specific embodiment, Y, or a conjugate as described herein comprising Y, specifically binds to a polypeptide of interest with an affinity (K_{d}) less than or equal to 20 mM. In particular embodiments, such binding is with an affinity (K_{d}) less than or equal to about 20 mM, about 10 mM, about 1 mM, about 100 μM, about 10 μM, about 1 μM, about 100 nM, about 10 nM, or about 1 nM. Unless otherwise noted, “binds,” “binds to,” “specifically binds” or “specifically binds to” in this context are used interchangeably.

In certain embodiments, for example, the polypeptide of interest is a cell surface receptor and Y comprises an antibody that binds to the cell surface protein, e.g., the extracellular domain of the cell surface receptor. In other embodiments, for example, the polypeptide of interest is a soluble, (e.g., secreted) polypeptide of interest, for example the ligand for a cell surface receptor polypeptide, and Y comprises an antibody that binds to the ligand.

Polypeptides may contain L-amino acids, D-amino acids, or both and may contain any of a variety of amino acid modifications or analogs known in the art. Useful modifications include, e.g., terminal acetylation, amidation, methylation, etc.

In certain embodiments, Y is a polypeptide that comprises about 10, about 20, about 30, about 40, about 50, about 100, about 150, about 200, about 250, about 300, about 350, about 400, about 450, about 500, about 550, about 600, about 650, about 700, about 750, about 800, about 850, about 900, or about 950 amino acids.

In certain embodiments, Y is a polypeptide comprises about 10-50, about 50-100, about 100-150, about 150-200, about 200-250, about 250-300, about 300-350, about 350-400, about 400-450, about 450-500, about 500-600, about 600-700, about 700-800, about 800-900, or about 900-1000 amino acids.

In certain embodiments, Y is an antibody (Ab). In certain embodiments, Ab is a monoclonal antibody. In certain embodiments, Ab is a human antibody. In certain embodiments, Ab is a humanized antibody. In certain embodiments, Ab is a chimeric antibody. In certain embodiments, Ab is a full-length antibody that comprises two heavy chains and two light chains. In particular embodiments, Ab is an IgG antibody, e.g., is an IgG1, IgG2, IgG3 or IgG4 antibody. In certain embodiments, Ab is a single chain antibody. In yet other embodiments, Ab is an antigen-binding fragment of an antibody, e.g., a Fab fragment.

In certain embodiments, the antibody specifically binds to a cancer antigen.

In certain embodiments, the antibody specifically binds to a hepatocyte antigen.

In certain embodiments, the antibody specifically binds to an antigen presented on a macrophage.

In certain embodiments, the antibody specifically binds to an intact complement or a fragment thereof. In certain embodiments, the antibody specifically binds to one or more immunodominant epitope(s) within intact complement or a fragment thereof.

In certain embodiments, the antibody specifically binds to a cell surface receptor. In certain embodiments, the antibody specifically binds to a cell surface receptor ligand.

In certain embodiments, the antibody specifically binds to an epidermal growth factor (EGF) protein, e.g., a human EGF. In certain embodiments, the antibody specifically binds to one or more immunodominant epitope(s) within an EGF protein.

In certain embodiments, the antibody specifically binds to an epidermal growth factor receptor (EGFR) protein, e.g., a human EGFR. In certain embodiments, the antibody specifically binds to one or more immunodominant epitope(s) within an EGFR protein. In a certain embodiment, the antibody comprises the CDRs present in cetuximab. In another certain embodiment, the antibody comprises the variable light chain and variable heavy chain present in cetuximab. In a particular embodiment, the antibody is cetuximab. In a certain embodiment, the antibody comprises the CDRs present in matuzumab. In another certain embodiment, the antibody comprises the variable light chain and variable heavy chain present in matuzumab. In a particular embodiment, the antibody is matuzumab.

In certain embodiments, the antibody specifically binds to vascular endothelial growth factor (VEGF) protein, e.g., human VEGF protein. In certain embodiments, the antibody specifically binds to one or more immunodominant epitope(s) within a VEGF protein.

In certain embodiments, the antibody specifically binds to a vascular endothelial growth factor receptor (VEGFR) protein, e.g., human VEGFR protein. In particular embodiments, the antibody specifically binds vascular endothelial growth factor receptor 2 (VEGFR2) protein, e.g., a human VEGFR2 protein. In other particular embodiments, the antibody specifically binds a vascular endothelial growth factor receptor 3 (VEGFR3) protein, e.g., a human VEGFR3 protein. In certain embodiments, the antibody specifically binds to one or more immunodominant epitope(s) within a VEGFR protein, a VEGFR2 protein or a VEGFR3 protein.

In certain embodiments, the antibody specifically binds to a fibroblast growth factor (FGF), e.g., a human FGF. In certain embodiments, the antibody specifically binds to one or more immunodominant epitope(s) within a FGF protein.

In certain embodiments, the antibody specifically binds to a fibroblast growth factor receptor (FGFR), e.g., a human FGFR. In particular embodiments, the antibody specifically binds fibroblast growth factor receptor 2 (FGFR2) protein, e.g., a human FGFR2 protein, for example, a FGFR2b protein. In other particular embodiments, the antibody specifically binds a fibroblast growth factor receptor 3 (FGFR3) protein, e.g., a human FGFR3 protein. In certain embodiments, the antibody specifically binds to one or more immunodominant epitope(s) within a FGFR protein, a FGFR2 protein or a FGFR3 protein. In a certain embodiment, the antibody comprises the CDRs present in vofatamab. In another certain embodiment, the antibody comprises the variable light chain and the variable heavy chain present in vofatamab. In a particular embodiment is vofatamab. In a certain embodiment, the antibody comprises the CDRs present in bemarituzumab. In another certain embodiment, the antibody comprises the variable light chain and the variable heavy chain present in bemarituzumab. In a particular embodiment is bemarituzumab.

In certain embodiments, the antibody specifically binds to a receptor tyrosine kinase cMET protein. In certain embodiments, the antibody specifically binds to one or more immunodominant epitope(s) within a receptor tyrosine kinase cMET protein. In certain embodiments, the antibody comprises the CDRs present in onartuzumab (MetMAb; see, e.g., CAS number 1133766-06-9). In certain embodiments, the antibody comprises the variable light chain and the heavy chain present in onartuzumab. In certain embodiments, the antibody is onartuzumab. In certain embodiments, the antibody comprises the CDRs present in emibetuzumab (LY2875358; see, e.g., CAS number 1365287-97-3). In certain embodiments, the antibody comprises the variable light chain and the heavy chain present in emibetuzumab. In certain embodiments, the antibody is emibetuzumab. In certain embodiments, the antibody specifically binds to a CD47 protein, e.g., a human CD47 protein. In certain embodiments, the antibody specifically binds to one or more immunodominant epitope(s) within a CD47 protein. In a certain embodiment, the antibody comprises the CDRs present in Hu5F9-G4 (5F9). In another certain embodiment, the antibody comprises the variable light chain and the variable heavy chain present in Hu5F9-G4 (5F9). In a particular embodiment is Hu5F9-G4 (5F9).

In certain embodiments, the antibody specifically binds to an immune checkpoint inhibitor. In certain embodiments, the antibody binds to one or more immunodominant epitope(s) within an immune checkpoint inhibitor.

In certain embodiments, the antibody specifically binds to a programmed death protein, e.g., a human PD-1. In certain embodiments, the antibody specifically binds to one or more immunodominant epitope(s) within PD-1 protein. In a certain embodiment, the antibody comprises the CDRs present in nivolumab. In another certain embodiment, the antibody comprises the variable light chain and variable heavy chain present in nivolumab. In a particular embodiment, the antibody is nivoumab. In a certain embodiment, the antibody comprises the CDRs present in pembrolizumab. In another certain embodiment, the antibody comprises the variable light chain and variable heavy chain present in pembrolizumab. In a particular embodiment, the antibody is pembrolizumab.

In certain embodiments, the antibody specifically binds to a programmed death ligand-1 (PD-L1) protein, e.g., a human PD-L1. In certain embodiments, the antibody specifically binds to one or more immunodominant epitope(s) within PD-L1 protein. In a certain embodiment, the antibody comprises the CDRs present in atezolizumab. In another certain embodiment, the antibody comprises the variable light chain and variable heavy chain present in atezolizumab. In a particular embodiment, the antibody is atezolizumab. In a certain embodiment, the antibody comprises the CDRs present in 29E.2A3 (BioXCell). In another certain embodiment, the antibody comprises the variable light chain and variable heavy chain present in 29E.2A3. In a particular embodiment, the antibody is 29E.2A3.

In certain embodiments, the antibody binds to TIM3. In certain embodiments, the antibody binds to one or more immunodominant epitope(s) within TIM3.

In certain embodiments, the antibody specifically binds to a lectin. In certain embodiments, the antibody specifically binds to one or more immunodominant epitope(s) within a lectin. In certain embodiments, the antibody binds to SIGLEC. In certain embodiments, the antibody binds to one or more immunodominant epitope(s) within SIGLEC. In certain embodiments, the antibody binds to a cytokine receptor. In certain embodiments, the antibody binds to a one or more immunodominant epitope(s) within cytokine receptor. In certain embodiments, the antibody binds to sIL6R. In certain embodiments, the antibody binds to one or more immunodominant epitope(s) within sIL6R. In certain embodiments, the antibody binds to a cytokine. In certain embodiments, the antibody binds to one or more immunodominant epitope(s) within a cytokine. In yet certain embodiments, the antibody binds to MCP-1, TNF (e.g., a TNFalpha), IL1a, IL1b, IL4, IL5, IL6, IL12/IL23, IL13, IL17 or p40. In yet certain embodiments, the antibody binds to one or more immunodominant epitope(s) within MCP-1, TNF (e.g., a TNFalpha), IL1a, IL1b, IL4, IL5, IL6, IL12/IL23, IL13, IL17 or p40.

In certain embodiments, the antibody binds to a major histocompatibility protein (e.g., a MHC class I or class II molecule). In certain embodiments, the antibody binds to one or more immunodominant epitope(s) within a major histocompatibility protein (e.g., a MHC class I or class II molecule). In certain embodiments, the antibody binds to beta 2 microglobulin. In certain embodiments, the antibody binds to one or more immunodominant epitope(s) within beta 2 microglobulin.

The heavy chain and light chain sequences of an exemplary anti-EGFR antibody (see, e.g., cetuximab, CAS number 205923-56-4) are shown in Table A.

TABLE A
Heavy chain
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK
(SEQ ID NO: 1)
Light chain
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 2)

The heavy chain and light chain sequences of an exemplary Fab fragment of an anti-EGFR antibody (see, e.g., matuzumab, NCBI Accession Nos. 3C09H_H and 3C09_L, CAS number 339186-68-4) are shown in Table B.

TABLE B
Heavy chain Fab
QVQLVQSGAEVKKPGASVKVSCKASGYTFTSHWMHWVRQAPGQGLEWIG
EFNPSNGRTNYNEKFKSKATMTVDTSTNTAYMELSSLRSEDTAVYYCAS
RDYDYAGRYFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKS
(SEQ ID NO: 3)
Light chain
DIQMTQSPSSLSASVGDRVTITCSASSSVTYMYWYQQKPGKAPKLLIYD
TSNLASGVPSRFSGSGSGTDYTFTISSLQPEDIATYYCQQWSSHIFTFG
QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGE
(SEQ ID NO: 4)

The heavy chain and light chain sequences of an exemplary anti-PD-L1 antibody (see, e.g., atezolizumab, CAS number 138723-44-3) are shown in Table C.

TABLE C
Heavy chain
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 5)
Light chain
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 6)

### 5.1.2.3 TNFα Binding Moieties

TNF-alpha (TNFα) is a soluble cytokine produced by monocytes and macrophages as part of immune and inflammatory processes and is involved in a diverse range of cellular responses including differentiation, proliferation, inflammation, and cell death. TNFα is a type II transmembrane protein that can be cleaved and secreted as a soluble form. Both the transmembrane and soluble biologically active forms of TNFα are homotrimeric complexes that can signal through TNF receptors 1 and 2 (TNF-R1 and TNF-R2). TNFα is directly involved in systemic inflammation through the regulation of the intracellular NF-κB, JNK and p38-MAPK signaling pathways.

A number of biologic agents (e.g., monoclonal antibody drugs) have been developed to inhibit TNF binding to TNF receptors and shown to be clinically effective in a number of autoinflammatory diseases, including rheumatoid arthritis (RA), psoriatic arthritis, ankylosing spondylitis, Crohn's disease, psoriasis, juvenile idiopathic arthritis, ulcerative colitis, hidradenitis suppurativa, and certain types of uveitis. However, small molecules drugs for TNFα that are capable of directly disrupting the high-affinity interaction of TNFα with its cognate receptor (e.g., TNF-R1) in vivo, with reduction in proinflammatory signaling response, have remained elusive.

Agents that target TNFα targeting having advantages over existing biologic and small molecule agents, in terms of mechanism of action, oral delivery, being nonimmunogenic, and/or more cost-effective are of interest, particularly for use in treating chronic indications associated with TNFα.

The TNFα binding moiety is a small molecule that specifically binds to TNFα. The TNFα binding moiety can be a TNFα inhibitor, such as a competitive inhibitor of TNF receptor binding or an allosteric inhibitor of TNF signaling. The compounds of this disclosure can include a potent TNFα inhibitor, e.g., an inhibitor having sub-micromolar inhibitory activity. In some embodiments, the TNFα inhibitor is an allosteric inhibitor. In some embodiments, the TNFα binding moiety is an allosteric desymmetrization TNFα inhibitor. An allosteric desymmetrization TNFα inhibitor refers to a compound that binds to an allosteric site within TNFα and stabilizes the trimeric unit in a nonsymmetrical conformation that allows the TNFα trimer to recruit only two out of the three copies of TNF Receptor (TNFR, e.g., TNFR1), leading to an incompetent TNFα-TNFR signaling complex.

See e.g., Xiao et al. in Journal of Medicinal Chemistry 2020 63 (23), 15050-15071, and McMillan et al. in Nature Communications (2021) 12:582, which discloses an analysis of the X-ray co-crystal structure of exemplary inhibitors bound to TNFα. Allosteric desymmetrization TNFα inhibitor can act via a particular mechanism of action to provide potent inhibitory activity. For example, (a) the TNFα inhibitor binding site is a cavity within the TNFα trimer created via movement of monomer A, (b) the inhibitor stabilizes the TNFα trimer in an inactive conformation by forming key π-π and hydrogen bonding interactions, (c) an allosteric desymmetrization TNFα inhibitor binds to TNFα trimer leading to major disruption of one TNFR binding site and minor disruption of a second site, while the third site remains unchanged, and (d) the allosteric desymmetrization TNFα inhibitor modulates TNF-R activity through an allosteric mechanism rather than direct competition with TNFR. Thus, the binding of an allosteric desymmetrization TNFα inhibitor to the symmetric TNFα trimer can lead to the formation of an asymmetric trimer which prevents the recruitment of three TNF receptor molecules that are necessary for signaling.

In some embodiments, the TNFα binding moiety (Y) of formula (I) is an allosteric desymmetrization TNFα inhibitor of formula (XII):

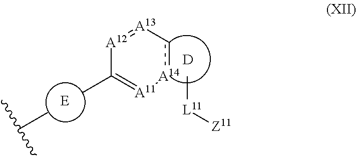

wherein:
A¹¹-A¹³ are independently selected from N and CR³⁰;
A¹⁴ is C or N;
D is a fused 5- or 6-membered ring selected from optionally substituted aryl, optionally substituted heteroaryl, and optionally substituted heterocycle;
E is selected from optionally substituted aryl, optionally substituted heteroaryl, optionally substituted cycloalkyl, and optionally substituted heterocycle;
L¹¹ is a linker comprising 1, 2 or 3 linking moieties each independently selected from optionally substituted (C₁-C₄)alkyl, —C(O)—, —O—, —S—, and —NR³⁶—;
Z¹¹ is a cyclic group selected from optionally substituted aryl, optionally substituted heteroaryl, optionally substituted cycloalkyl, and optionally substituted heterocycle;
each R³⁰ is independently selected from H, halogen (e.g., F), OH, optionally substituted (C₁-C₆)alkyl, optionally substituted (C₁-C₆)alkoxy, and CN; and
each R³⁶ is independently selected from H, and optionally substituted (C₁-C₆)alkyl.

In some embodiments of formula (XII), one of A¹¹-A¹³ is N. In some embodiments of formula (XII), two of A¹¹-A¹³ are N. In some embodiments of formula (XII), at least two of A¹¹-A¹³ are independently CR³⁰. In some embodiments of formula (XII), A¹¹ is N.

In some embodiments of formula (XII), D is a fused 6-membered heterocycle, such as a fused pyridyl ring.

In some embodiments of formula (XII), D is a fused 5-membered heterocycle, such as a fused imidazole ring.

### 4-Amino-Quinoline or Naphthyridine Compounds

Allosteric desymmetrization TNFα inhibitors of interest which can be incorporated into bifunctional compounds of this disclosure include, but are not limited to, TNFα inhibitors including substituted 4-aminoquinoline compounds and substituted 4-aminonaphthyridine compounds described by Xiao et al. in Journal of Medicinal Chemistry 2020 63 (23), 15050-15071, and related compounds described in International publication WO2017/023905 and U.S. Pat. No. 10,865,191, the disclosures of which are herein incorporated by reference in their entirety.

In some embodiments, the TNFα binding moiety of formula (XII), is of formula (XIII):

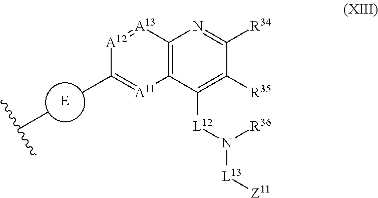

wherein:
A¹¹ is CR³¹ or N;
A¹² is CR³² or N;
A¹³ is CR³³ or N;
L¹² is a bond or —CH₂—;
L¹² is a bond or optionally substituted (C₁-C₃)alkyl;
E is a cyclic group selected from dihydropyridinonyl, phenyl, piperidinyl, pyrazinyl, pyrazolyl, pyridinyl, and pyrimidinyl, wherein the cyclic group E is optionally substituted;
Z¹¹ is a cyclic group selected from C₃₋₆ cycloalkyl, cyclopentenyl, phenyl, furanyl, imidazolyl, indolinyl, isoquinolinyl, isothiazolyl, isoxazolyl, oxazolyl, piperidinyl, pyrazinyl, pyrazolyl, pyridinyl, pyrimidinyl, thiazolyl, and thiophenyl, wherein the cyclic group Z¹¹ is optionally substituted; and
R³¹⁻³⁵ are independently selected from H, halogen, —CN, NO₂, optionally substituted C₁₋₆ alkyl, C₁₋₆ haloalkyl (e.g., —CF₃), and optionally substituted C₁₋₆ alkoxy (e.g., —OCF₃).

In some embodiments of formula (XIII), Y is of formula (XIV):

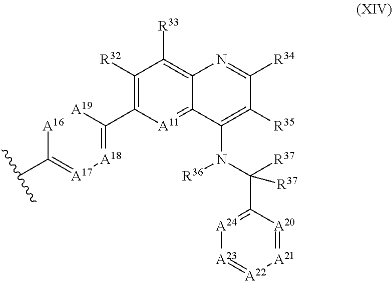

wherein:
A¹⁶ to A²⁴ are independently selected from CR⁵¹ and N;
each R³⁷ is independently selected from H, halogen (e.g., F), and optionally substituted (C₁-C₆)alkyl; and
each R⁵¹ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹, wherein each R²¹ is independently selected from H, and optionally substituted (C₁-C₆)alkyl.

In some embodiments of formula (XIV), Y is of formula (XV):

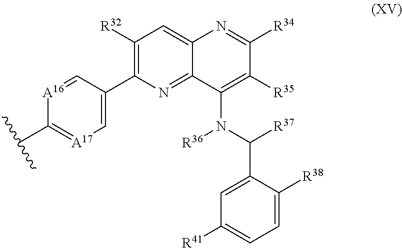

wherein:
A¹⁶ and A¹⁷ are independently selected from N and CH;
R³⁸ and R⁴¹ are independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
R³⁷ is selected from H, halogen (e.g., F), and optionally substituted (C₁-C₆)alkyl.

In some embodiments of formula (XIV)—(XV), R³² is halogen. In some embodiments of formula (XIV)—(XV), R³² is F.

In some embodiments of formula (XIII)—(XV), R³⁴ and R³⁵ are independently selected from H, halogen and optionally substituted (C₁-C₆)alkyl. In some embodiments of formula (XIII)—(XV), R³⁴ is (C₁-C₃)alkyl, and R³⁵ is halogen. In some embodiments of formula (XIII)—(XV), R³⁴ is methyl, and R³⁵ is chloride.

In some embodiments of formula (XIII)—(XV), R³⁷ is not H, and the carbon atom to which R³⁷ is attached is chiral and has an I configuration.

In some embodiments of formula (XIII)—(XV), R³⁷ is (C₁-C₃)alkyl. In some embodiments of formula (XIII)—(V), R³⁷ is methyl.

In some embodiments of formula (XIII)—(XV), R³⁶ is H.

In some embodiments of formula (XIII)—(XV), R′ is halogen. In some embodiments of formula (XIII)—(XV), R³⁸ is F.

In some embodiments of formula (XIII)—(XV), R⁴¹ is CN.

In some embodiments of formula (XV), Y is of formula (Xva):

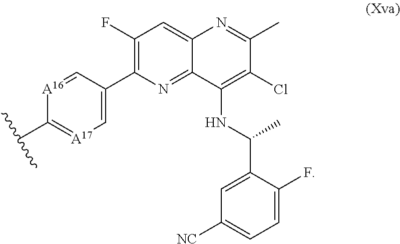

In some embodiments of formula (XV)-(Xva), A¹⁶ and A¹⁷ are each N. In some embodiments of formula (XV)-(Xva), A¹⁶ is CH and A¹⁷ is N. In some embodiments of formula (XV)-(Xva), A¹⁶ and A¹⁷ are each CH.

### Benzimidazole and Imidazopyridine Inhibitors

Allosteric desymmetrization TNFα inhibitors of interest which can be incorporated into bifunctional compounds of this disclosure include, but are not limited to, benzimidazole and imidazopyridine TNFα inhibitor compounds described by O'Connell et al. in Nature Communications (2019) 10:5795, and in U.S. Pat. Nos. 9,550,737 and 9,309,243, the disclosures of which are herein incorporated by reference in their entirety.

In some embodiments, the TNFα binding moiety of formula (I), is of formula (XVI):

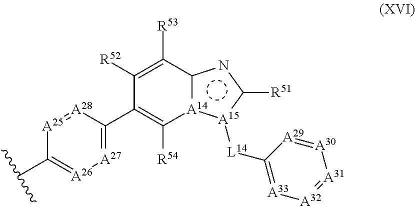

wherein:
A¹⁴ is C and A⁵ is N, or A¹⁴ is N and A¹⁵ is C;
A²⁵ to A³³ are independently selected from N and CR⁵⁵;
L¹⁴ is selected from —N(R⁵⁶)—and optionally substituted methylene (e.g., L¹⁴ is selected from —N(R⁵⁶)—, —CH₂—, —CH(OH)—, —CH(OCH₃)—, —CH(OCH₂CO₂H)—, —CH(NH₂)—, —CH(NHCOCH₃)—, —CH(CO₂H)—, —CH(CO₂benzyl)-, —CH(CH₃)—or —C(CH₃)(OH)—);
R⁵¹ is selected from H, halogen, optionally substituted (C₁-C₆)alkyl (e.g., CF₃), optionally substituted aryl, optionally substituted heteroaryl, optionally substituted cycloalkyl, and optionally substituted heterocycle;
R⁵²-R⁵⁵ are each independently selected from H, halogen (e.g., F), OH, optionally substituted (C₁-C₆)alkyl (e.g., CF₃), optionally substituted (C₁-C₆)alkoxy, and CN; and
R⁵⁶ is selected from H, and optionally substituted (C₁-C₆)alkyl.

In some embodiments of formula (XVI), Y is of formula (XVII) or (XVIII):

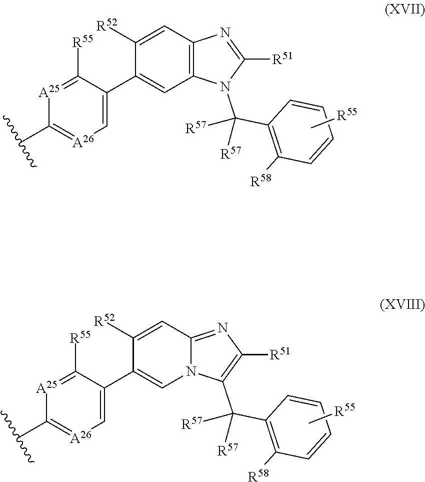

wherein each R⁵⁷ is independently selected from H, and optionally substituted (C₁-C₆)alkyl; and R⁵⁸ is selected from H, halogen (e.g., F), OH, optionally substituted (C₁-C₆)alkyl (e.g., CF₃), optionally substituted (C₁-C₆)alkoxy, and CN.

In some embodiments of formula (XVII) or (XVIII), Y is of formula (XVIIa)-(XVIIIa):

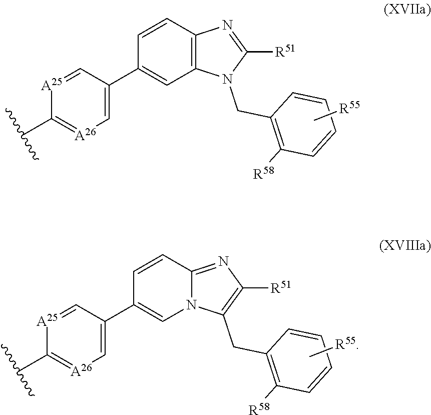

In some embodiments of formula (XVI)—(XVIIIa), R⁵⁸ is C₁₋₆ haloalkoxy. In some embodiments of formula (XVI)—(XVIIIa), R⁵⁸ is —OCHF₂.

In some embodiments of formula (XVI)—(XVIIIa), A²⁵ and A²⁶ are each N.

In some embodiments of formula (XVI)—(XVIIIa), A²⁵ is CH and A²⁶ is N.

In some embodiments of formula (XVI)—(XVIIIa), A²⁵ and A²⁶ are each CH.

In some embodiments of formula (XVI)—(XVIIIa), R⁵¹ is —C₁₋₆ alkyl-O-(optionally substituted aryl). In some embodiments, the optionally substituted aryl is optionally substituted phenyl. In some embodiments, the optionally substituted aryl is phenyl substituted with an optionally substituted heterocycle. In some embodiments of formula (XVI)—(XVIIIa), R⁵¹ is —CH₂—O-phenyl, where the phenyl is optionally further substituted with a heterocyclic group.

In some embodiments of formula (XVI)—(XVIIIa), R⁵¹ is —C₁₋₆ alkyl. In some embodiments of formula (XVI)—(XVIIIa), R⁵¹ is methyl or ethyl.

In some embodiments of formula (XVI)—(XVIIIa), Y has one of the following structures:

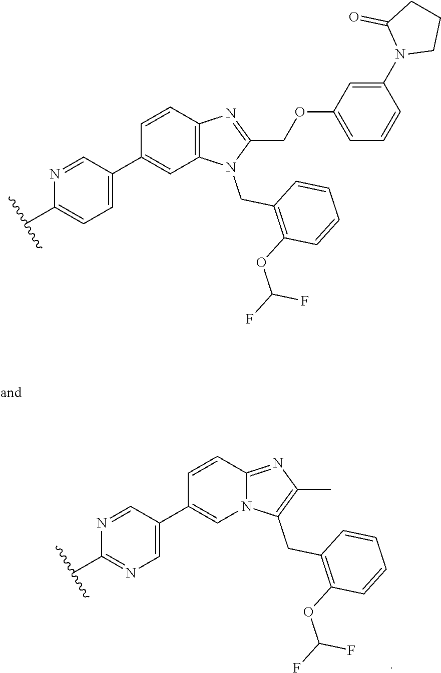 [SMILES: *c1ccc(-c2ccc3nc(COc4cccc(N5CCCC5=O)c4)n(Cc4ccccc4OC(F)F)c3c2)cn1 ; *c1ncc(-c2ccc3nc(C)c(Cc4ccccc4OC(F)F)n3c2)cn1]

### Indazolone and Indolinone Inhibitors

Allosteric desymmetrization TNFα inhibitors of interest which can be incorporated into bifunctional compounds of this disclosure include, but are not limited to, indazolone and indolinone TNFα inhibitor compounds described by Dietrich et al. in J. Medicinal Chemistry, 2021, 64, 1, 417-429, and TNFα inhibitor compounds of International publication WO2016168633 and U.S. Pat. No. 10,160,748, the disclosures of which are herein incorporated by reference in their entirety.

In some embodiments, the TNFα binding moiety of formula (I) is of formula (XXIa):

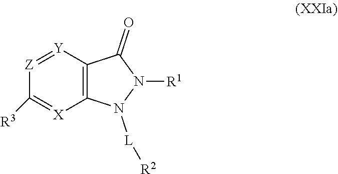

or a pharmaceutically acceptable salt thereof, wherein
X, Y and Z are independently CR⁴ or N, provided that Y and Z are not both N;
L is a bond, optionally substituted (C₁-C₃)alkylene or —C(O)—;
R¹ is H, CD₃, optionally substituted (C₁-C₃)alkyl, or optionally substituted (C₃-C₆)cycloalkyl;
R² is optionally substituted aryl (e.g., optionally substituted phenyl, such as 2-(halomethoxy)-phenyl) or optionally substituted heteroaryl (e.g., optionally substituted 3-pyridyl, such as 2-cyano-3-pyridyl);
R³ is —R^{3a}—R^{3b}, wherein:
   R^{3a} is an optionally substituted saturated, unsaturated or partially saturated heterocyclyl or optionally substituted heteroaryl;
   R^{3b} is —N(R^{a})(R^{b}), —O(R^{a}), optionally substituted (C₁-C₅)alkyl, optionally substituted (C₃-C₆)cycloalkyl, or —(CH₂)ₚ— optionally substituted heterocyclyl; wherein
      R^{a} and R^{b} are independently selected from H, optionally substituted (C₁-C₅)alkyl, and —(CH₂)n-optionally substituted heterocyclyl;
R⁴ is independently H, Cl, CN, F, CF₃, or optionally substituted (C₁-C₃)alkyl;
n is 0 or 1; and
p is 0, 1 or 2.

In some embodiments, the TNFα binding moiety of formula (I) is of formula (XXIb):

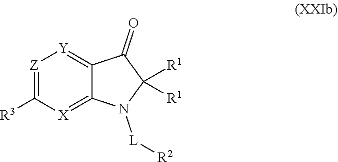

or a pharmaceutically acceptable salt thereof, wherein
X, Y and Z are independently CR⁴ or N, provided that Y and Z are not both N;
L is a bond, optionally substituted (C₁-C₃)alkylene or —C(O)—;
each R¹ is independently H, optionally substituted (C₁-C₃)alkyl, or optionally substituted (C₃-C₆)cycloalkyl;
R² is optionally substituted aryl or optionally substituted heteroaryl;
R³ is —R^{3a}—R^{3b}, wherein:
   R^{3a} is an optionally substituted saturated, unsaturated or partially saturated heterocyclyl or optionally substituted heteroaryl;
   R^{3b} is —N(R^{a})(R^{b}), —O(R^{a}), optionally substituted (C₁-C₅)alkyl, optionally substituted (C₃—C)cycloalkyl, or —(CH₂)ₚ— optionally substituted heterocyclyl; wherein
      R^{a} and R^{b} are independently selected from H, optionally substituted (C₁-C₅)alkyl, and —(CH₂)n-optionally substituted heterocyclyl;
R⁴ is independently H, Cl, CN, F, CF₃, or optionally substituted (C₁-C₃)alkyl;
n is 0 or 1; and
p is 0, 1 or 2.

### 5.2. Example Compounds

This disclosure includes compounds of formula (I) and (IIIA)-(IIIB) which can include:
(1) one or more particular folate binding ligand (X) of formula (Ia) (e.g., as described herein, such as ligands in Tables 1 or 2),
(2) a linker including one or more linking moieties (e.g., as described herein, such as any one or more of the linking moieties of Tables 3); and
(3) a moiety of interest (Y) e.g., as described herein, such as any one of the groups of (Table 4).

Tables 5-8 illustrate several example folate receptor binding compounds of this disclosure. It is understood that this disclosure includes Y (e.g., as described herein) conjugates of each of the exemplary compounds of Tables 5-8. For example, conjugates where the group Y has been conjugated to a different Y, such as a biomolecule or a small molecule ligand for a target protein.

The Y groups of such compounds can be utilized to connect to another Y moiety of interest (e.g., as described below). It is understood that any of these compounds can also be prepared de novo to include an alternative Y moiety of interest (e.g., as described herein below) rather than the Y groups depicted. In some embodiments, such compounds are referred to as a conjugate, e.g., a biomolecule conjugate that specifically binds a target protein.

TABLE 5
Example folate receptor binder-linker compounds

| # | Compound structure |
|---|---|
| I-1 | 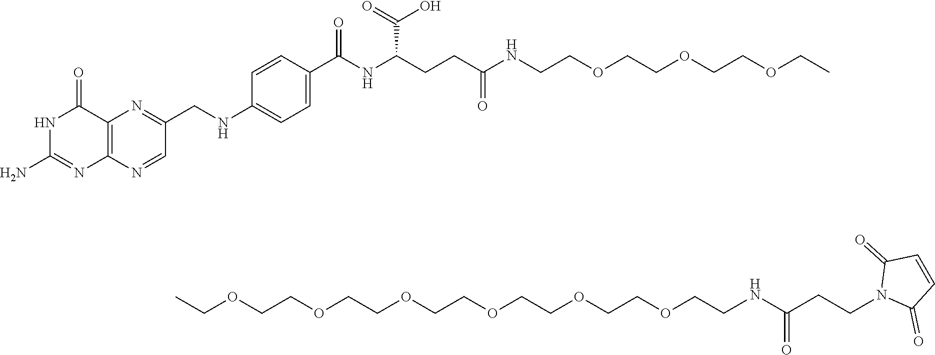 |
| I-2 | 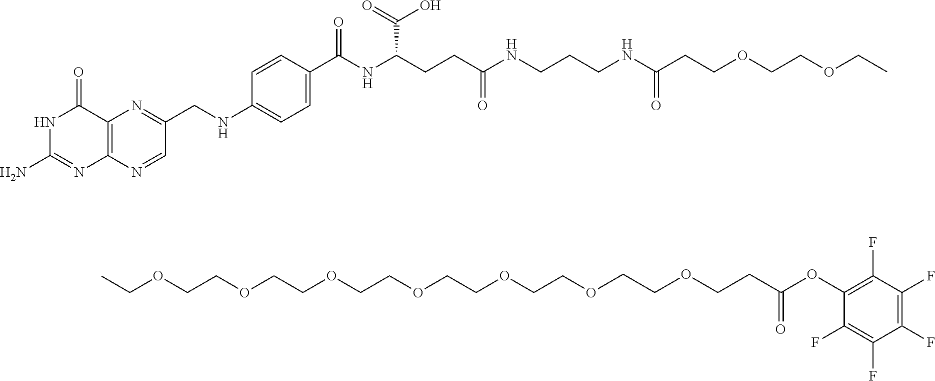 |
| I-3 | 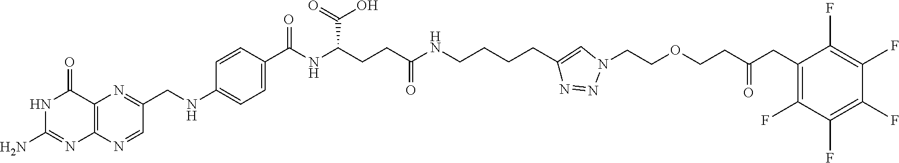 [SMILES: Nc1nc2ncc(CNc3ccc(C(=O)N[C@@H](CCC(=O)NCCCCc4cn(CCOCCC(=O)Cc5c(F)c(F)c(F)c(F)c5F)nn4)C(=O)O)cc3)nc2c(=O)[nH]1] |
| I-4 | 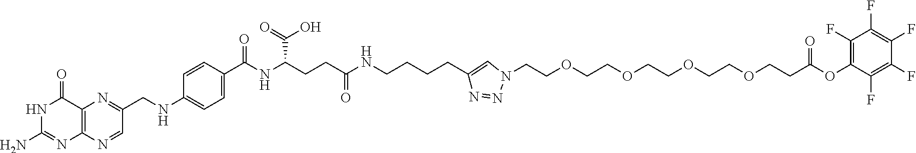 [SMILES: Nc1nc2ncc(CNc3ccc(C(=O)N[C@@H](CCC(=O)NCCCCc4cn(CCOCCOCCOCCOCCC(=O)Oc5c(F)c(F)c(F)c(F)c5F)nn4)C(=O)O)cc3)nc2c(=O)[nH]1] |
| I- 4B | 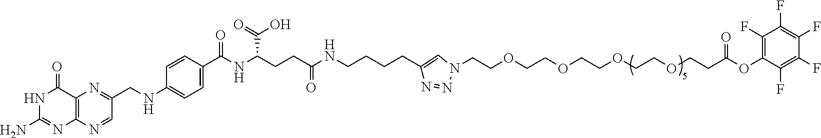 |
| I- 4C | 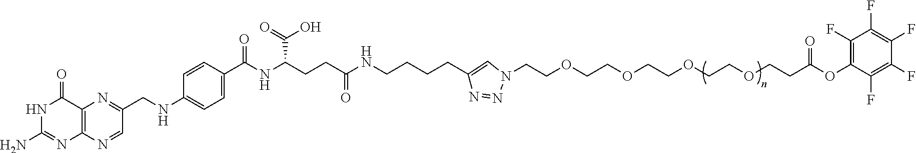 |
| | where n is 0, 1, 2, 3, 4, 5, or 6 |
| I-5 | 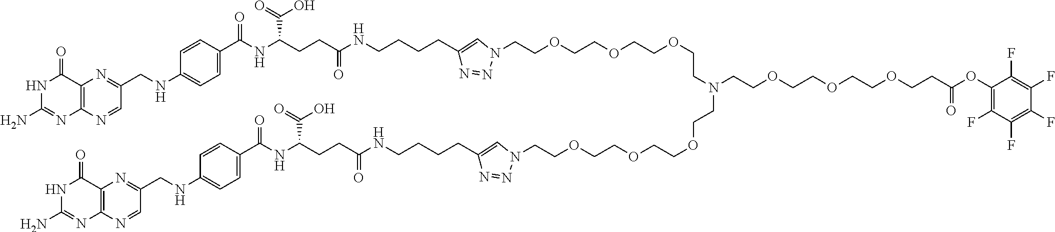 [SMILES: Nc1nc2ncc(CNc3ccc(C(=O)N[C@@H](CCC(=O)NCCCCc4cn(CCOCCOCCOCCN(CCOCCOCCOCCC(=O)Oc5c(F)c(F)c(F)c(F)c5F)CCOCCOCCOCCn5cc(CCCCNC(=O)CC[C@H](NC(=O)c6ccc(NCc7cnc8nc(N)[nH]c(=O)c8n7)cc6)C(=O)O)nn5)nn4)C(=O)O)cc3)nc2c(=O)[nH]1] |
| I-6 | 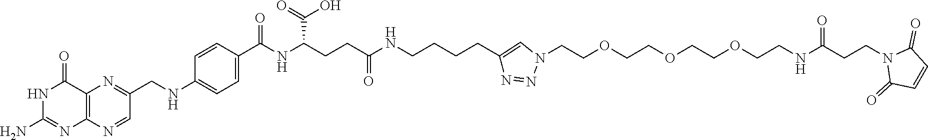 [SMILES: Nc1nc2ncc(CNc3ccc(C(=O)N[C@@H](CCC(=O)NCCCCc4cn(CCOCCOCCOCCNC(=O)CCN5C(=O)C=CC5=O)nn4)C(=O)O)cc3)nc2c(=O)[nH]1] |
| I-7 | 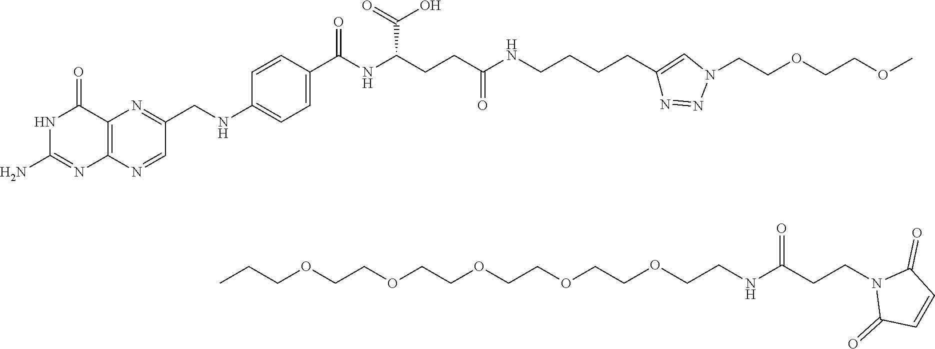 |
| I-8 | 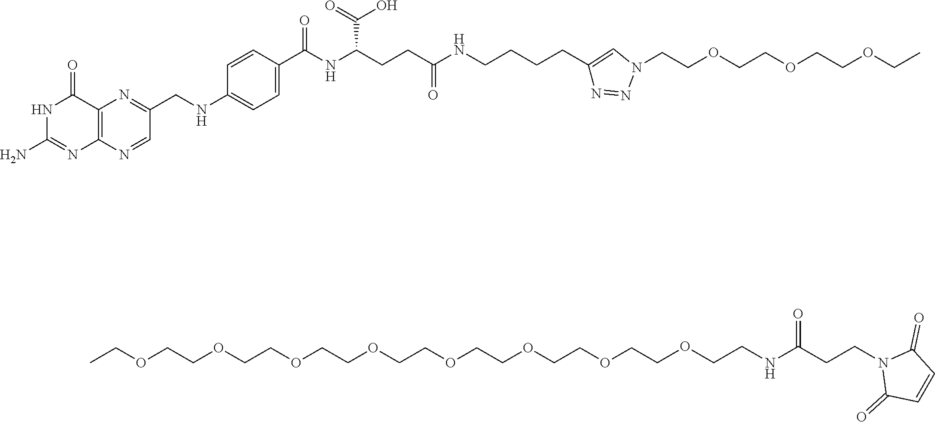 |
| I-9 | 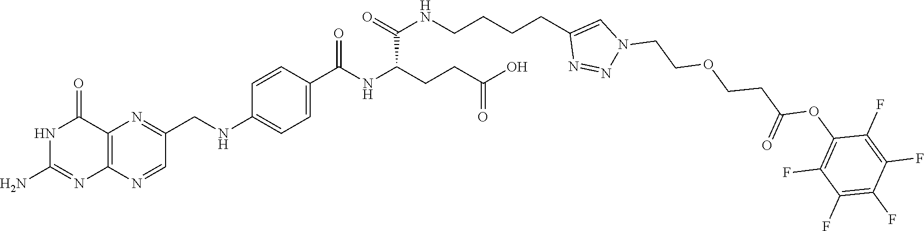 [SMILES: Nc1nc2ncc(CNc3ccc(C(=O)N[C@@H](CCC(=O)O)C(=O)NCCCCc4cn(CCOCCC(=O)Oc5c(F)c(F)c(F)c(F)c5F)nn4)cc3)nc2c(=O)[nH]1] |
| I- 10 | 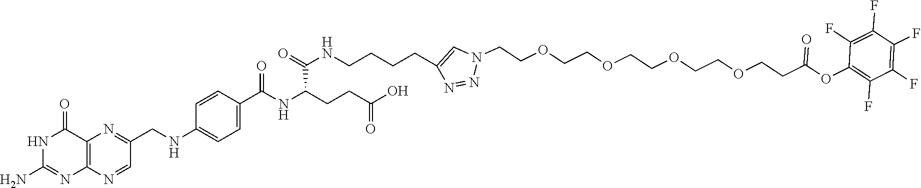 [SMILES: Nc1nc2ncc(CNc3ccc(C(=O)N[C@@H](CCC(=O)O)C(=O)NCCCCc4cn(CCOCCOCCOCCOCCC(=O)Oc5c(F)c(F)c(F)c(F)c5F)nn4)cc3)nc2c(=O)[nH]1] |
| I- 10B | 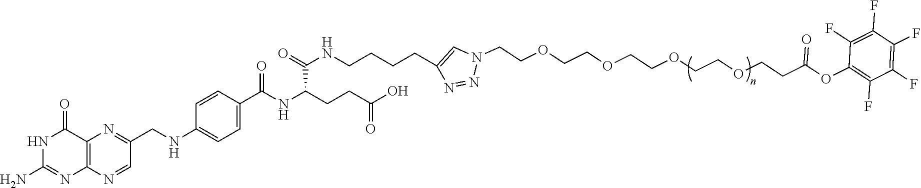 |
| | where n is 0, 1, 2, 3, 4, 5, or 6. |
| I- 76 | 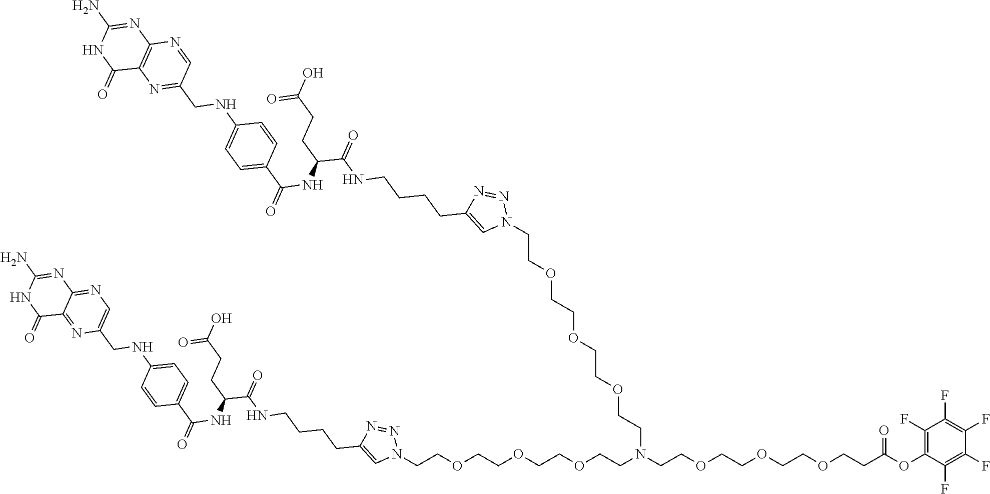 [SMILES: Nc1nc2ncc(CNc3ccc(C(=O)N[C@@H](CCC(=O)O)C(=O)NCCCCc4cn(CCOCCOCCOCCN(CCOCCOCCOCCC(=O)Oc5c(F)c(F)c(F)c(F)c5F)CCOCCOCCOCCn5cc(CCCCNC(=O)[C@H](CCC(=O)O)NC(=O)c6ccc(NCc7cnc8nc(N)[nH]c(=O)c8n7)cc6)nn5)nn4)cc3)nc2c(=O)[nH]1] |
| I- 77 | 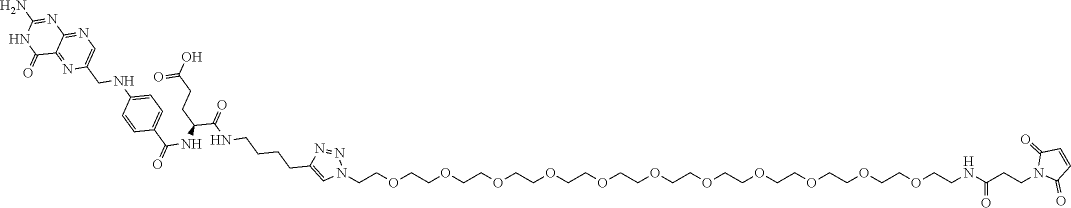 [SMILES: Nc1nc2ncc(CNc3ccc(C(=O)N[C@@H](CCC(=O)O)C(=O)NCCCCc4cn(CCOCCOCCOCCOCCOCCOCCOCCOCCOCCOCCOCCNC(=O)CCN5C(=O)C=CC5=O)nn4)cc3)nc2c(=O)[nH]1] |

TABLE 6
Exemplary folate receptor binding TNF-alpha targeting compounds of Formula (IIIB)

| # | Compound structure |
|---|---|
| I- 11 | 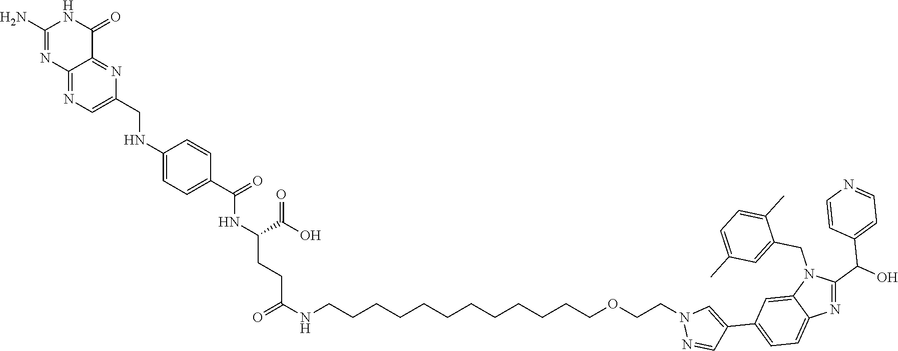 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(NCc6cnc7nc(N)[nH]c(=O)c7n6)cc5)C(=O)O)c4)cc32)c1] |
| I- 12 | 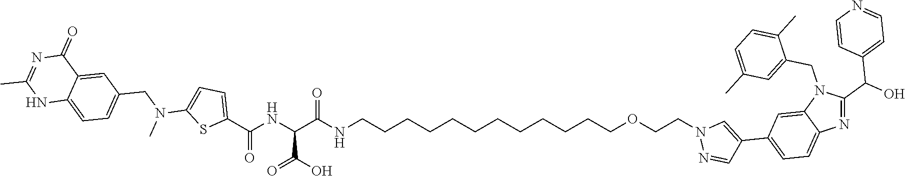 [SMILES: Cc1ccc(C)c(Cn2c(C(O)c3ccncc3)nc3ccc(-c4cnn(CCOCCCCCCCCCCCCNC(=O)[C@H](NC(=O)c5ccc(N(C)Cc6ccc7[nH]c(C)nc(=O)c7c6)s5)C(=O)O)c4)cc32)c1] |
| I- 13 | 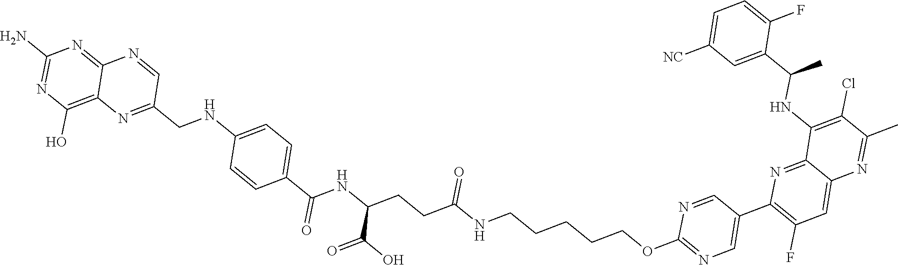 [SMILES: Cc1nc2cc(F)c(-c3cnc(OCCCCCNC(=O)CC[C@H](NC(=O)c4ccc(NCc5cnc6nc(N)nc(O)c6n5)cc4)C(=O)O)nc3)nc2c(N[C@H](C)c2cc(C#N)ccc2F)c1Cl] |
| I- 14 | 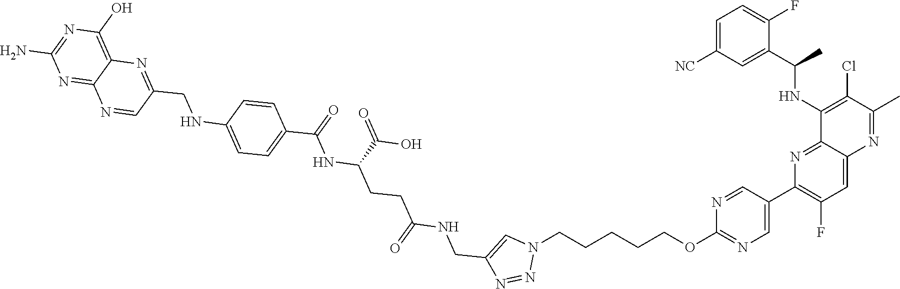 [SMILES: Cc1nc2cc(F)c(-c3cnc(OCCCCCn4cc(CNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 15 | 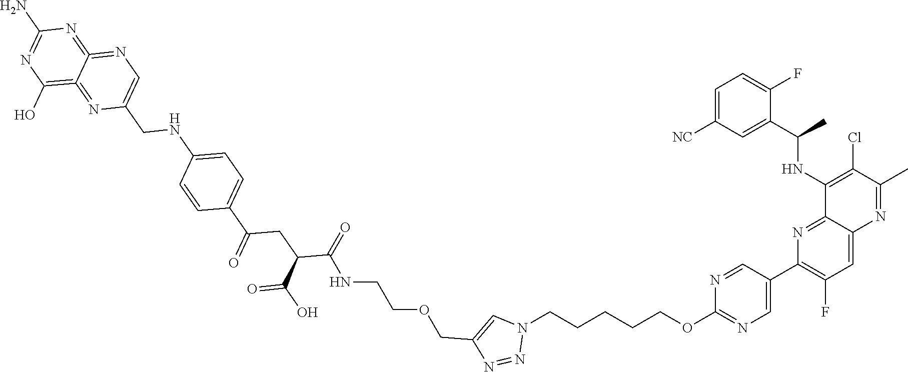 |
| I- 16 | 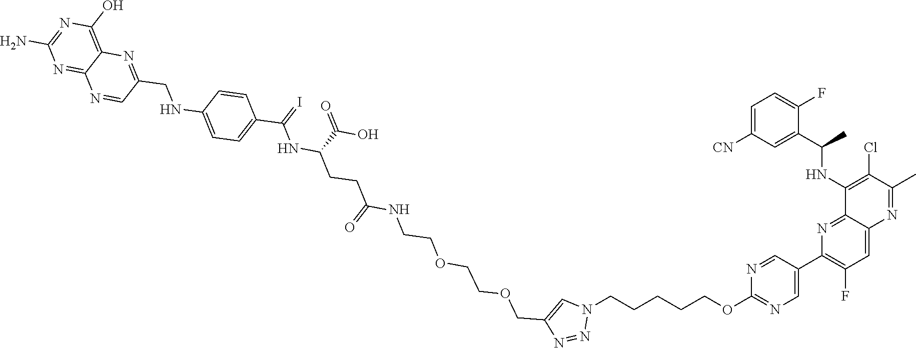 |
| I- 17 | 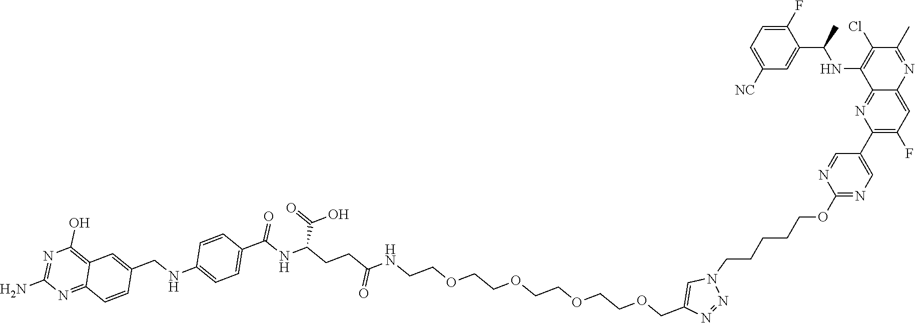 [SMILES: Cc1nc2cc(F)c(-c3cnc(OCCCCCn4cc(COCCOCCOCCOCCNC(=O)CC[C@H](NC(=O)c5ccc(NCc6ccc7nc(N)nc(O)c7c6)cc5)C(=O)O)nn4)nc3)nc2c(N[C@H](C)c2cc(C#N)ccc2F)c1Cl] |
| I- 18 | 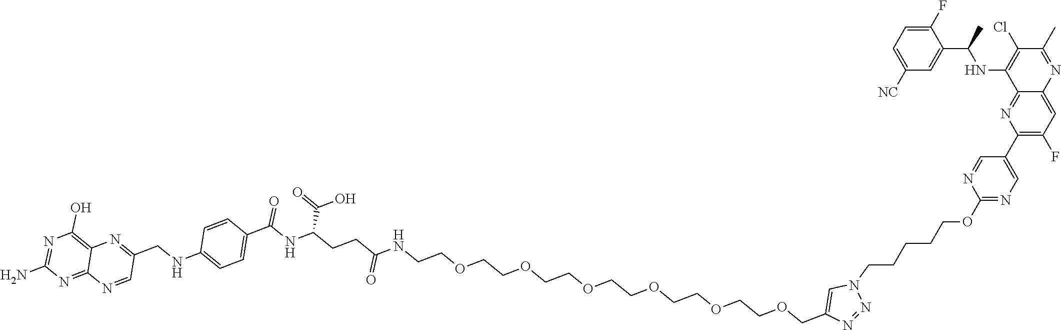 [SMILES: Cc1nc2cc(F)c(-c3cnc(OCCCCCn4cc(COCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 19 | 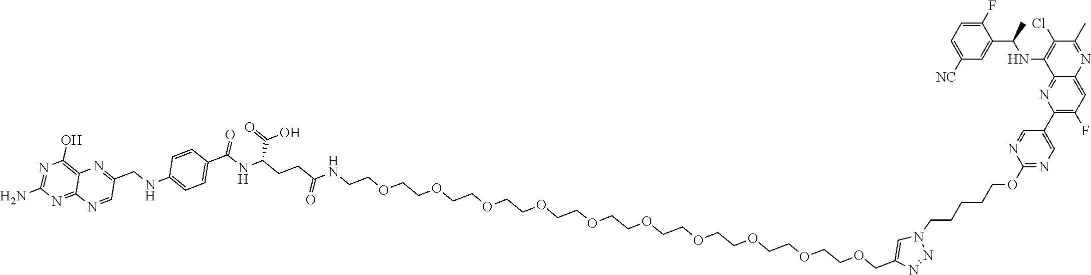 [SMILES: Cc1nc2cc(F)c(-c3cnc(OCCCCCn4cc(COCCOCCOCCOCCOCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 20 | 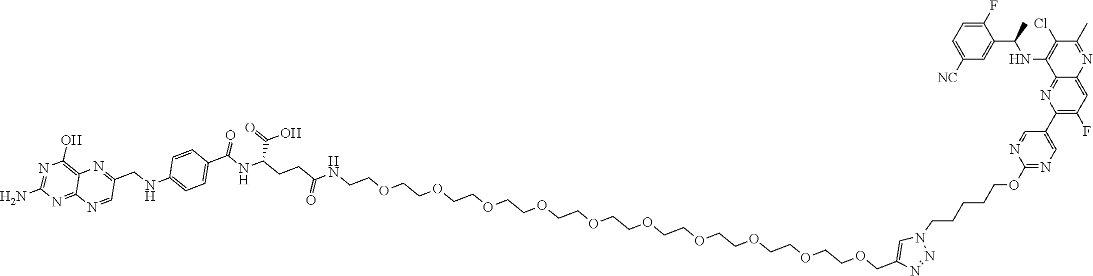 [SMILES: Cc1nc2cc(F)c(-c3cnc(OCCCCCn4cc(COCCOCCOCCOCCOCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 22 | 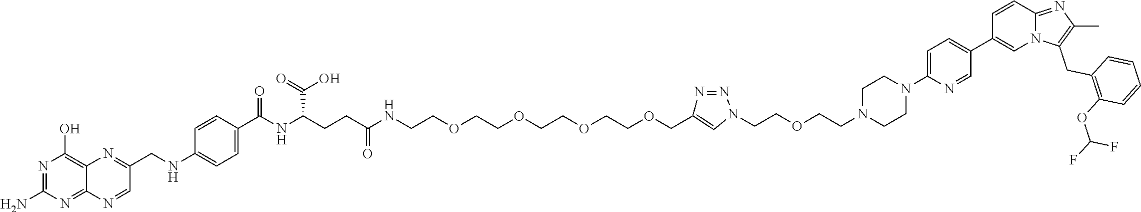 [SMILES: Cc1nc2ccc(-c3ccc(N4CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)cn2c1Cc1ccccc1OC(F)F] |
| I- 23 | 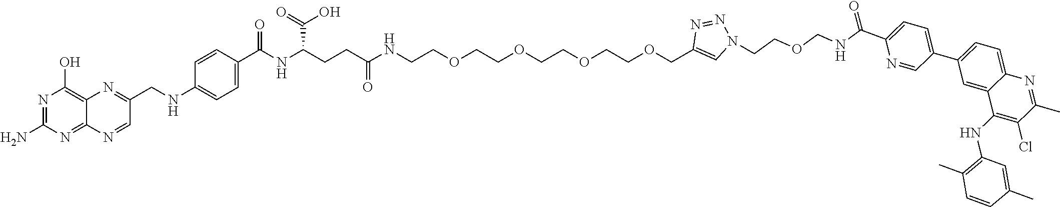 [SMILES: Cc1ccc(C)c(Nc2c(Cl)c(C)nc3ccc(-c4ccc(C(=O)NCOCCn5cc(COCCOCCOCCOCCNC(=O)CC[C@H](NC(=O)c6ccc(NCc7cnc8nc(N)nc(O)c8n7)cc6)C(=O)O)nn5)nc4)cc23)c1] |
| I- 24 | 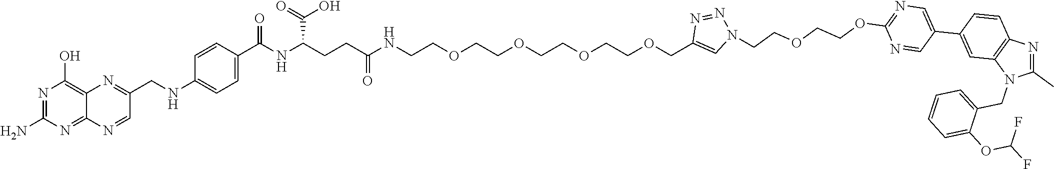 [SMILES: Cc1nc2ccc(-c3cnc(OCCOCCn4cc(COCCOCCOCCOCCNC(=O)CC[C@H](NC(=O)c5ccc(NCc6cnc7nc(N)nc(O)c7n6)cc5)C(=O)O)nn4)nc3)cc2n1Cc1ccccc1OC(F)F] |
| I- 25 | 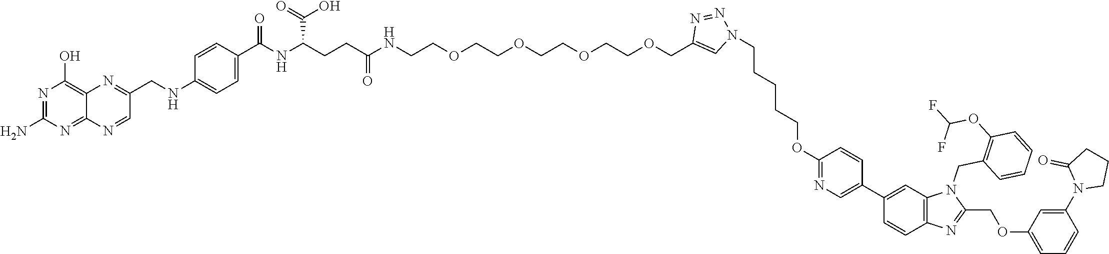 [SMILES: Nc1nc(O)c2nc(CNc3ccc(C(=O)N[C@@H](CCC(=O)NCCOCCOCCOCCOCc4cn(CCCCCOc5ccc(-c6ccc7nc(COc8cccc(N9CCCC9=O)c8)n(Cc8ccccc8OC(F)F)c7c6)cn5)nn4)C(=O)O)cc3)cnc2n1] |
| I- 26 | 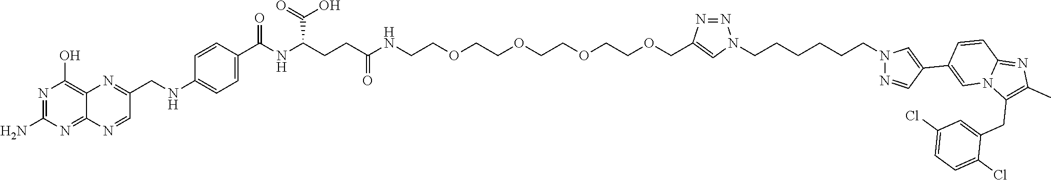 [SMILES: Cc1nc2ccc(-c3cnn(CCCCCCn4cc(COCCOCCOCCOCCNC(=O)CC[C@H](NC(=O)c5ccc(NCc6cnc7nc(N)nc(O)c7n6)cc5)C(=O)O)nn4)c3)cn2c1Cc1cc(Cl)ccc1Cl] |
| I- 27 | 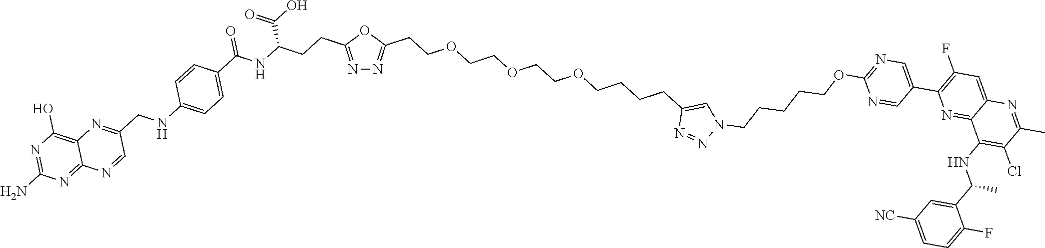 [SMILES: Cc1nc2cc(F)c(-c3cnc(OCCCCCn4cc(CCCCOCCOCCOCCc5nnc(CC[C@H](NC(=O)c6ccc(NCc7cnc8nc(N)nc(O)c8n7)cc6)C(=O)O)o5)nn4)nc3)nc2c(N[C@H](C)c2cc(C#N)ccc2F)c1Cl] |
| I- 28 | 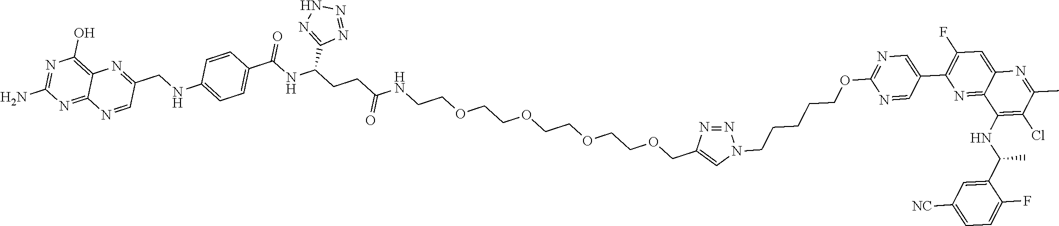 [SMILES: Cc1nc2cc(F)c(-c3cnc(OCCCCCn4cc(COCCOCCOCCOCCNC(=O)CC[C@H](NC(=O)c5ccc(NCc6cnc7nc(N)nc(O)c7n6)cc5)c5nn[nH]n5)nn4)nc3)nc2c(N[C@H](C)c2cc(C#N)ccc2F)c1Cl] |
| I- 29 | 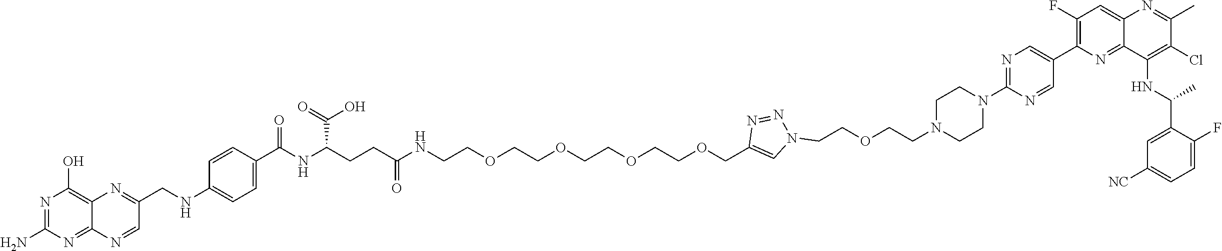 [SMILES: Cc1nc2cc(F)c(-c3cnc(N4CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 30 | 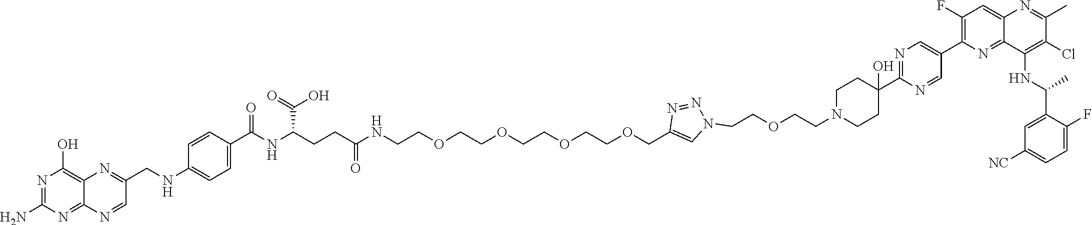 [SMILES: Cc1nc2cc(F)c(-c3cnc(C4(O)CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 31 | 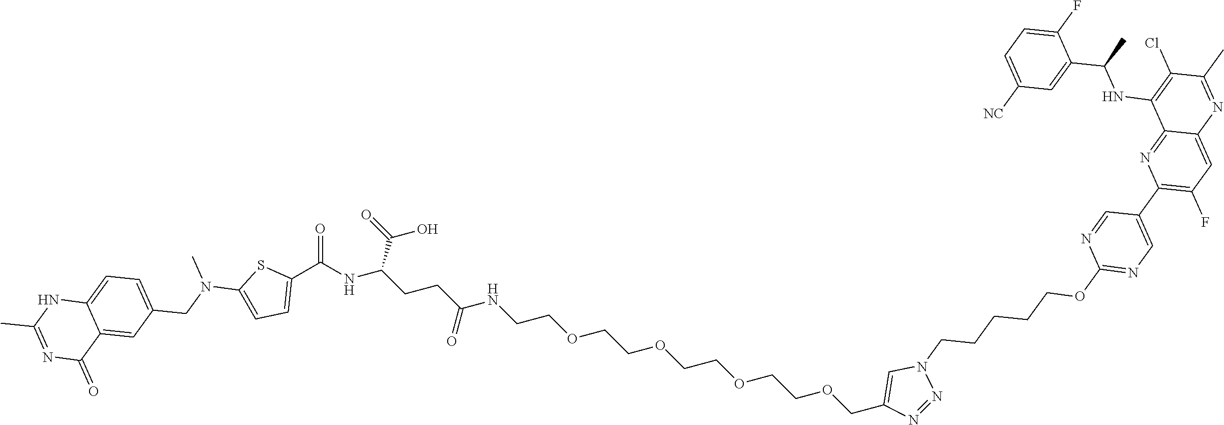 |
| I- 33 | 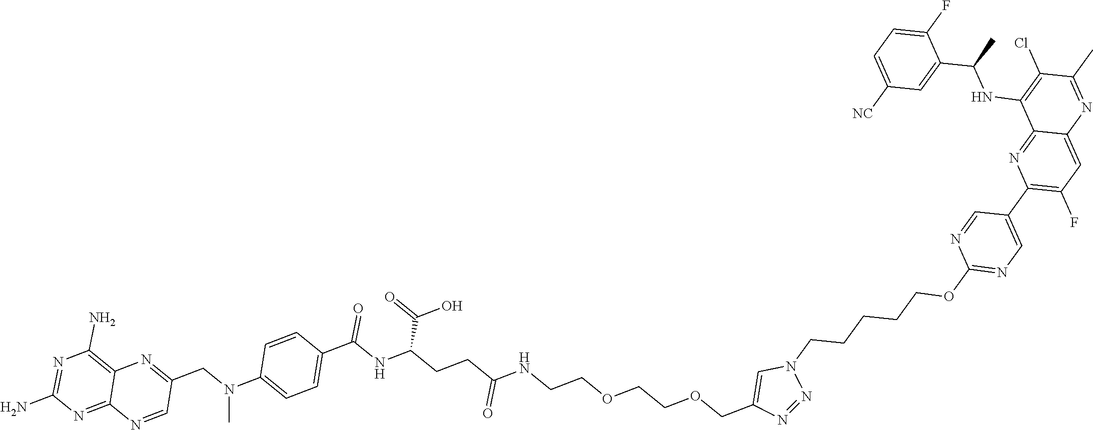 [SMILES: Cc1nc2cc(F)c(-c3cnc(OCCCCCn4cc(COCCOCCNC(=O)CC[C@H](NC(=O)c5ccc(N(C)Cc6cnc7nc(N)nc(N)c7n6)cc5)C(=O)O)nn4)nc3)nc2c(N[C@H](C)c2cc(C#N)ccc2F)c1Cl] |
| I- 36 | 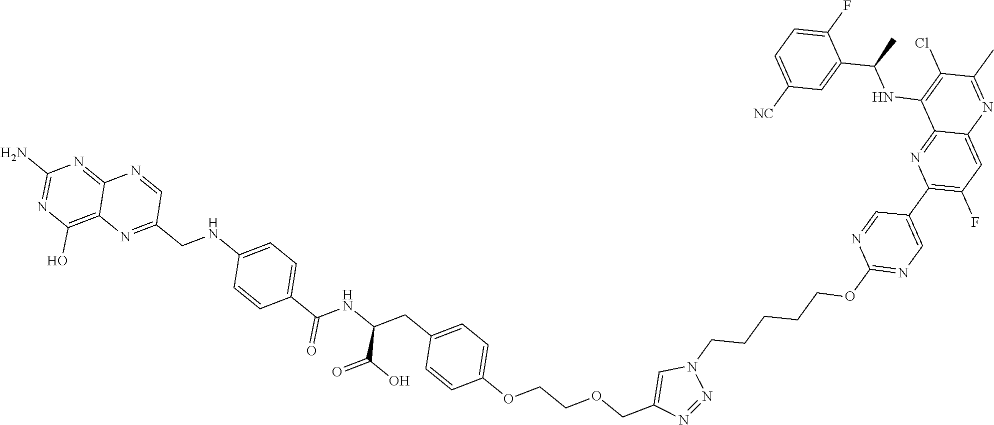 [SMILES: Cc1nc2cc(F)c(-c3cnc(OCCCCCn4cc(COCCOc5ccc(C[C@H](NC(=O)c6ccc(NCc7cnc8nc(N)nc(O)c8n7)cc6)C(=O)O)cc5)nn4)nc3)nc2c(N[C@H](C)c2cc(C#N)ccc2F)c1Cl] |
| I- 40 | 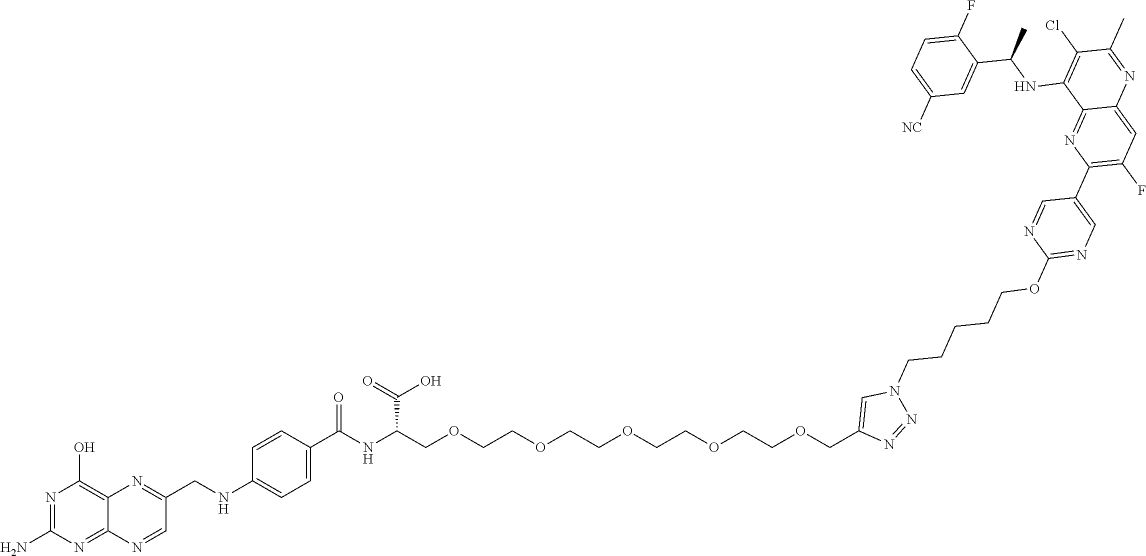 [SMILES: Cc1nc2cc(F)c(-c3cnc(OCCCCCn4cc(COCCOCCOCCOCCOC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 41 | 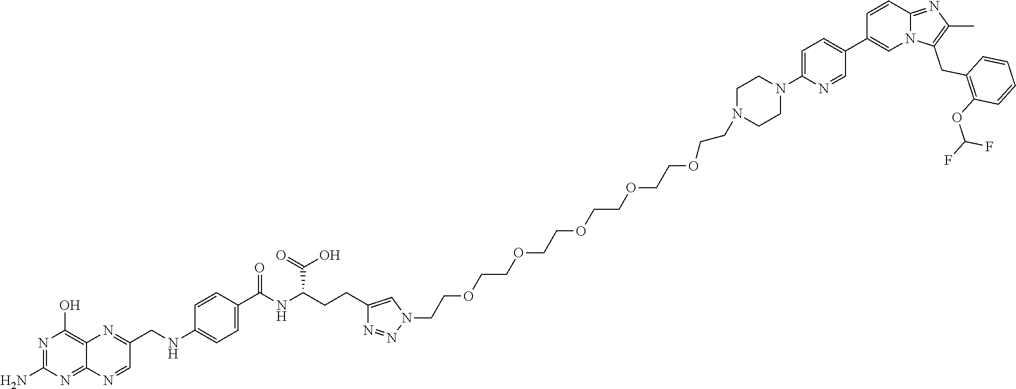 [SMILES: Cc1nc2ccc(-c3ccc(N4CCN(CCOCCOCCOCCOCCOCCn5cc(CC[C@H](NC(=O)c6ccc(NCc7cnc8nc(N)nc(O)c8n7)cc6)C(=O)O)nn5)CC4)nc3)cn2c1Cc1ccccc1OC(F)F] |
| I- 42 | 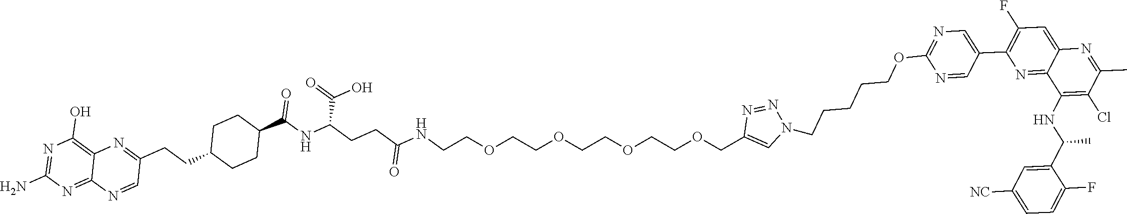 [SMILES: Cc1nc2cc(F)c(-c3cnc(OCCCCCn4cc(COCCOCCOCCOCCNC(=O)CC[C@H](NC(=O)[C@H]5CC[C@H](CCc6cnc7nc(N)nc(O)c7n6)CC5)C(=O)O)nn4)nc3)nc2c(N[C@H](C)c2cc(C#N)ccc2F)c1Cl] |
| I- 43 | 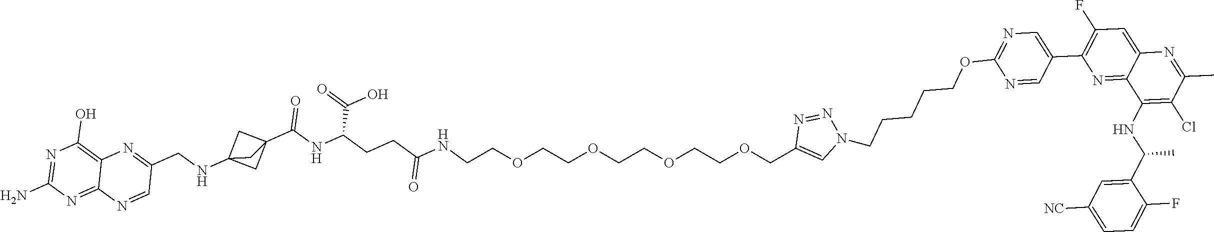 [SMILES: Cc1nc2cc(F)c(-c3cnc(OCCCCCn4cc(COCCOCCOCCOCCNC(=O)CC[C@H](NC(=O)C56CC(NCc7cnc8nc(N)nc(O)c8n7)(C5)C6)C(=O)O)nn4)nc3)nc2c(N[C@H](C)c2cc(C#N)ccc2F)c1Cl] |
| I- 44 | 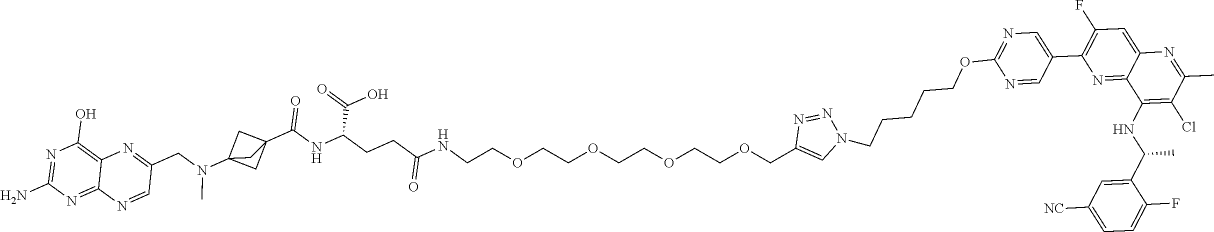 [SMILES: Cc1nc2cc(F)c(-c3cnc(OCCCCCn4cc(COCCOCCOCCOCCNC(=O)CC[C@H](NC(=O)C56CC(N(C)Cc7cnc8nc(N)nc(O)c8n7)(C5)C6)C(=O)O)nn4)nc3)nc2c(N[C@H](C)c2cc(C#N)ccc2F)c1Cl] |
| I- 45 | 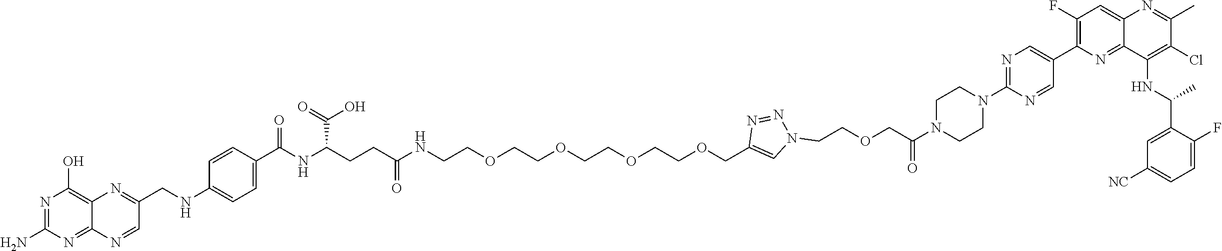 [SMILES: Cc1nc2cc(F)c(-c3cnc(N4CCN(C(=O)COCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 46 | 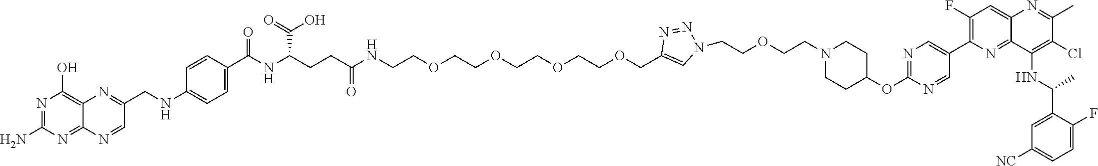 [SMILES: Cc1nc2cc(F)c(-c3cnc(OC4CCN(CCOCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 47 | 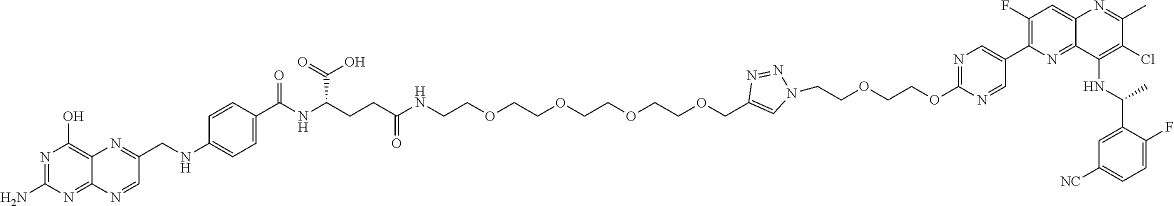 [SMILES: Cc1nc2cc(F)c(-c3cnc(OCCOCCn4cc(COCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 48 | 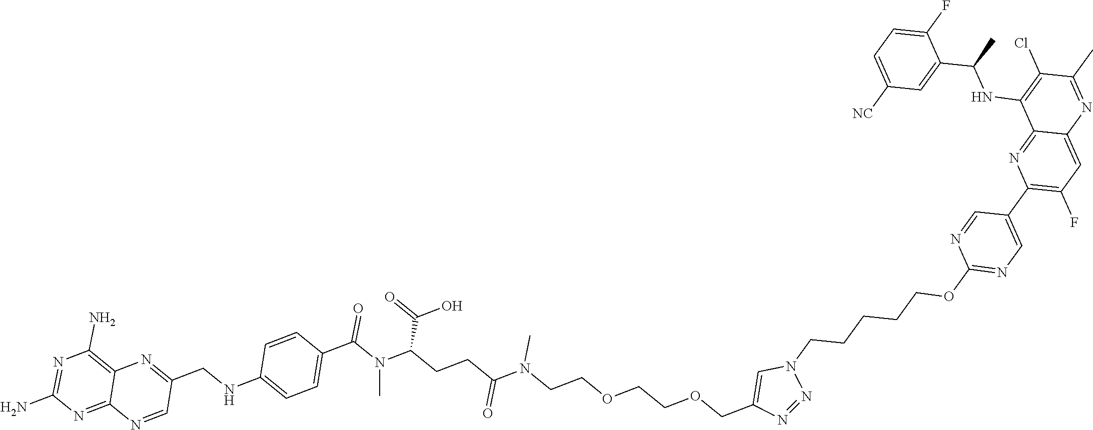 [SMILES: Cc1nc2cc(F)c(-c3cnc(OCCCCCn4cc(COCCOCCN(C)C(=O)CC[C@@H](C(=O)O)N(C)C(=O)c5ccc(NCc6cnc7nc(N)nc(N)c7n6)cc5)nn4)nc3)nc2c(N[C@H](C)c2cc(C#N)ccc2F)c1Cl] |
| I- 49 | 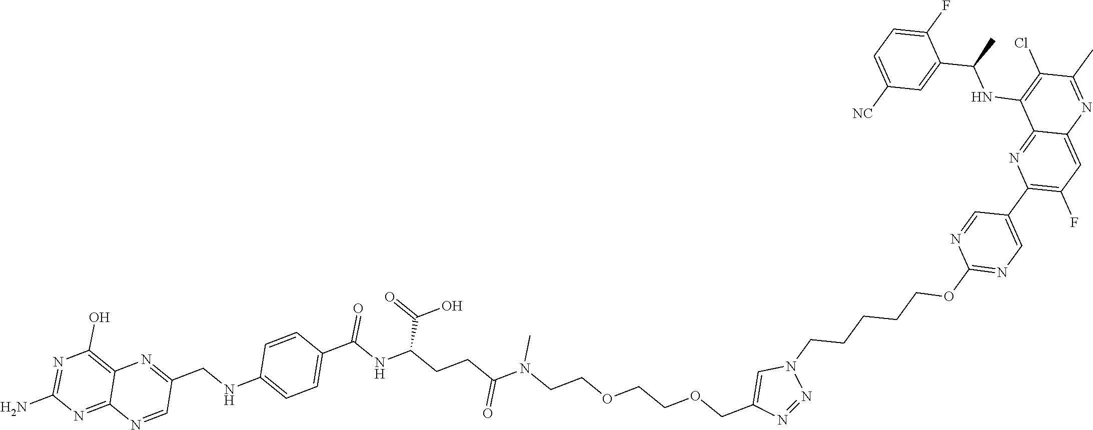 [SMILES: Cc1nc2cc(F)c(-c3cnc(OCCCCCn4cc(COCCOCCN(C)C(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| I- 50 | 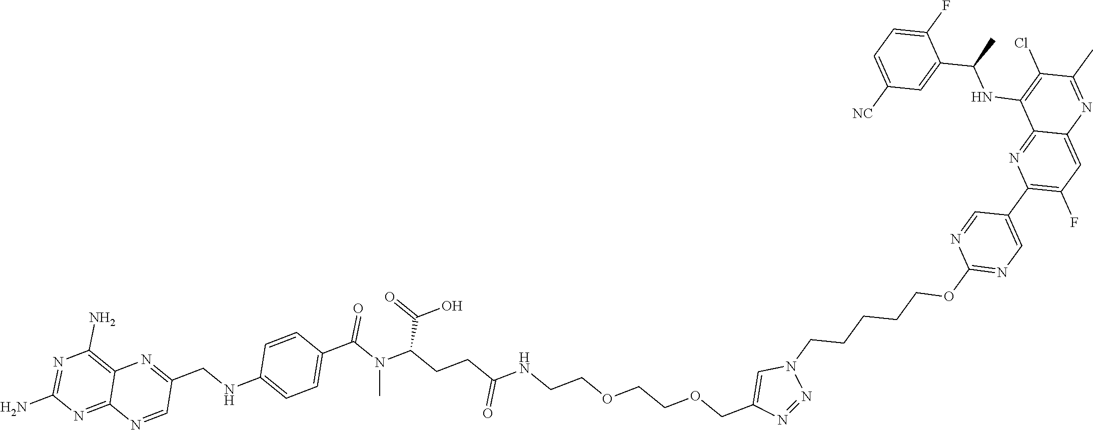 [SMILES: Cc1nc2cc(F)c(-c3cnc(OCCCCCn4cc(COCCOCCNC(=O)CC[C@@H](C(=O)O)N(C)C(=O)c5ccc(NCc6cnc7nc(N)nc(N)c7n6)cc5)nn4)nc3)nc2c(N[C@H](C)c2cc(C#N)ccc2F)c1Cl] |
| I- 51 | 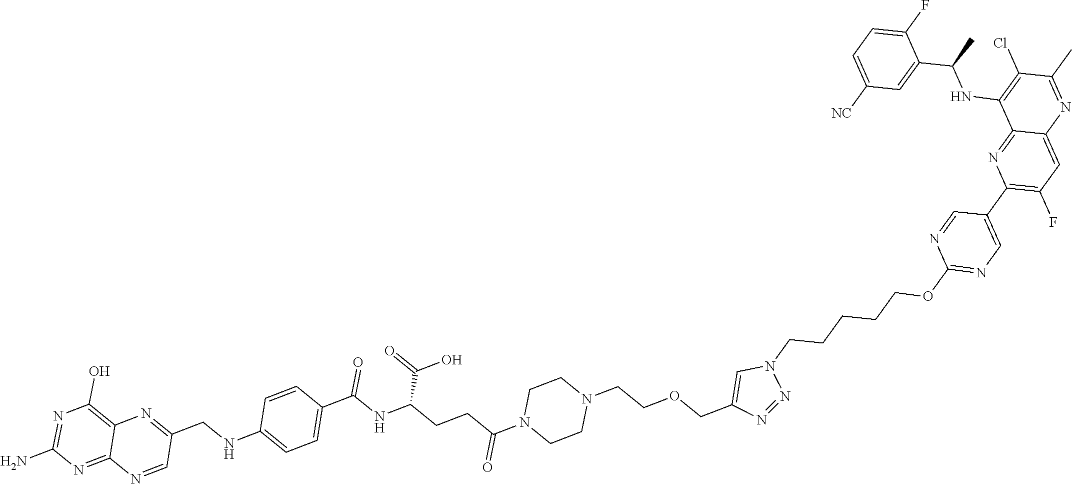 [SMILES: Cc1nc2cc(F)c(-c3cnc(OCCCCCn4cc(COCCN5CCN(C(=O)CC[C@H](NC(=O)c6ccc(NCc7cnc8nc(N)nc(O)c8n7)cc6)C(=O)O)CC5)nn4)nc3)nc2c(N[C@H](C)c2cc(C#N)ccc2F)c1Cl] |
| I- 52 | 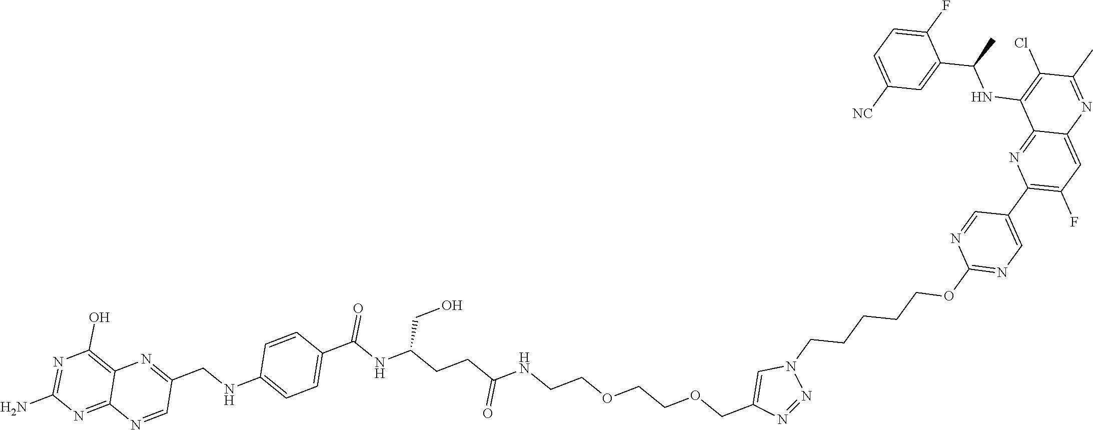 [SMILES: Cc1nc2cc(F)c(-c3cnc(OCCCCCn4cc(COCCOCCNC(=O)CC[C@@H](CO)NC(=O)c5ccc(NCc6cnc7nc(N)nc(O)c7n6)cc5)nn4)nc3)nc2c(N[C@H](C)c2cc(C#N)ccc2F)c1Cl] |
| I- 53 | 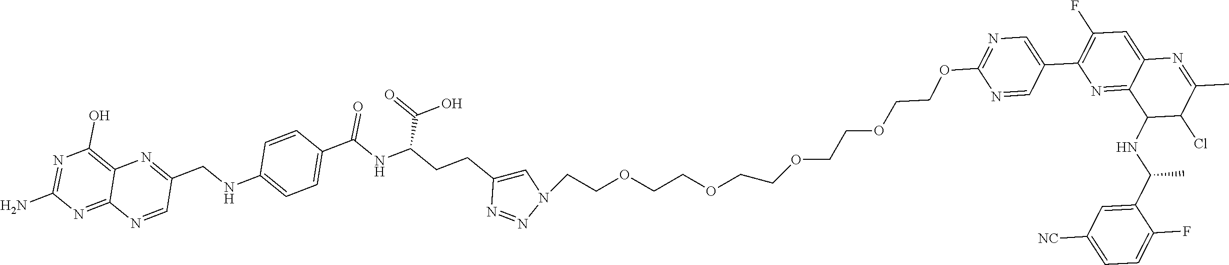 [SMILES: CC1=Nc2cc(F)c(-c3cnc(OCCOCCOCCOCCOCCn4cc(CC[C@H](NC(=O)c5ccc(NCc6cnc7nc(N)nc(O)c7n6)cc5)C(=O)O)nn4)nc3)nc2C(N[C@H](C)c2cc(C#N)ccc2F)C1Cl] |
| I- 54 | 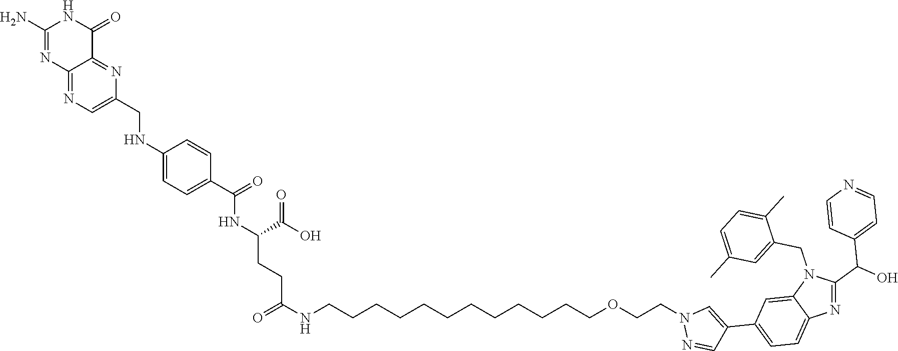 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(NCc6cnc7nc(N)[nH]c(=O)c7n6)cc5)C(=O)O)c4)cc32)c1] |
| I- 55 | 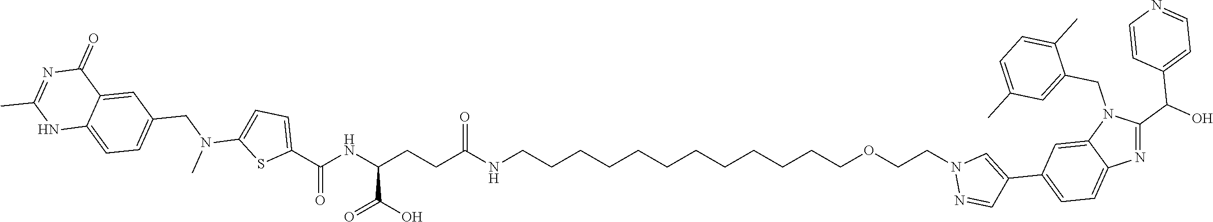 [SMILES: Cc1ccc(C)c(Cn2c(C(O)c3ccncc3)nc3ccc(-c4cnn(CCOCCCCCCCCCCCCNC(=O)CC[C@H](NC(=O)c5ccc(N(C)Cc6ccc7[nH]c(C)nc(=O)c7c6)s5)C(=O)O)c4)cc32)c1] |
| I- 56 | 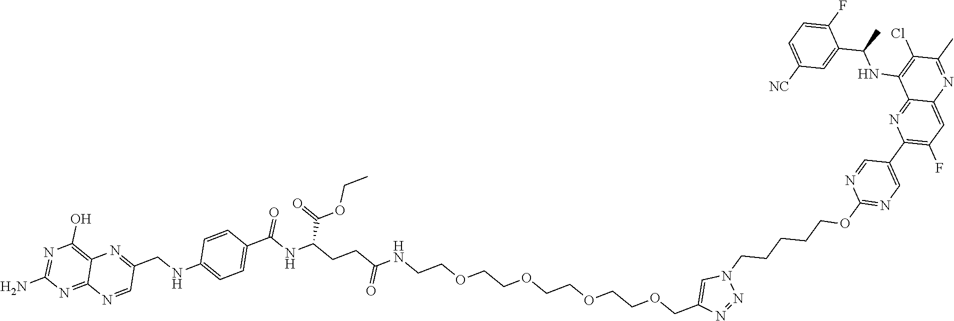 [SMILES: CCOC(=O)[C@H](CCC(=O)NCCOCCOCCOCCOCc1cn(CCCCCOc2ncc(-c3nc4c(N[C@H](C)c5cc(C#N)ccc5F)c(Cl)c(C)nc4cc3F)cn2)nn1)NC(=O)c1ccc(NCc2cnc3nc(N)nc(O)c3n2)cc1] |
| I- 57 | 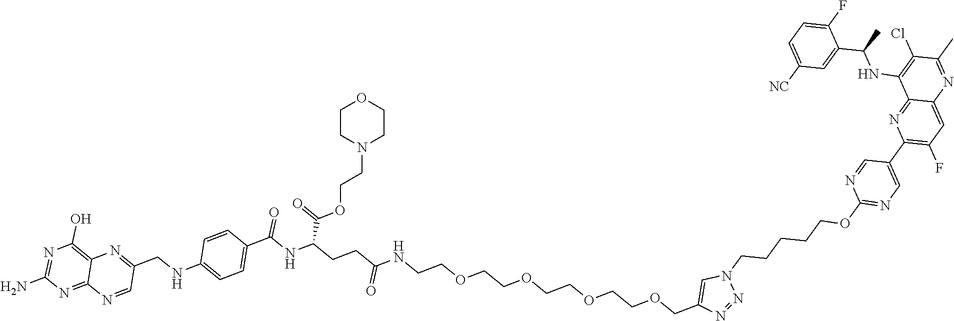 [SMILES: Cc1nc2cc(F)c(-c3cnc(OCCCCCn4cc(COCCOCCOCCOCCNC(=O)CC[C@H](NC(=O)c5ccc(NCc6cnc7nc(N)nc(O)c7n6)cc5)C(=O)OCCN5CCOCC5)nn4)nc3)nc2c(N[C@H](C)c2cc(C#N)ccc2F)c1Cl] |
| I- 58 | 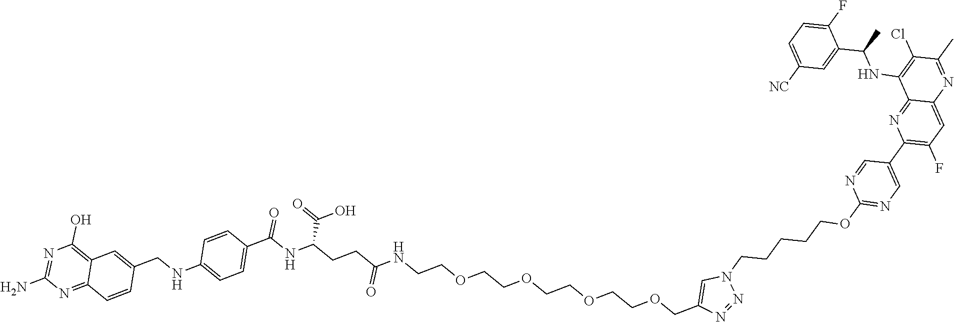 [SMILES: Cc1nc2cc(F)c(-c3cnc(OCCCCCn4cc(COCCOCCOCCOCCNC(=O)CC[C@H](NC(=O)c5ccc(NCc6ccc7nc(N)nc(O)c7c6)cc5)C(=O)O)nn4)nc3)nc2c(N[C@H](C)c2cc(C#N)ccc2F)c1Cl] |
| I- 59 | 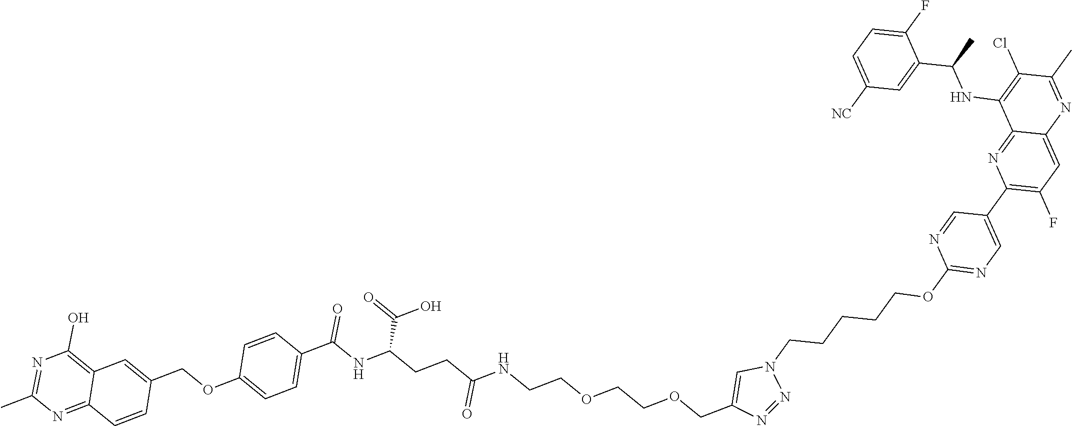 |
| I- 60 | 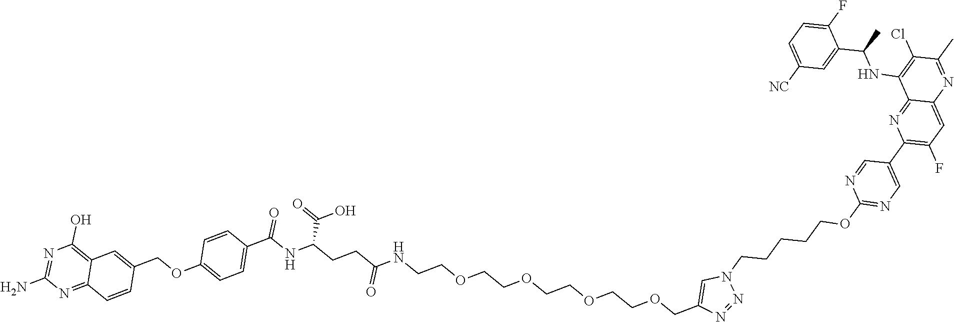 [SMILES: Cc1nc2cc(F)c(-c3cnc(OCCCCCn4cc(COCCOCCOCCOCCNC(=O)CC[C@H](NC(=O)c5ccc(OCc6ccc7nc(N)nc(O)c7c6)cc5)C(=O)O)nn4)nc3)nc2c(N[C@H](C)c2cc(C#N)ccc2F)c1Cl] |
| I- 61 | 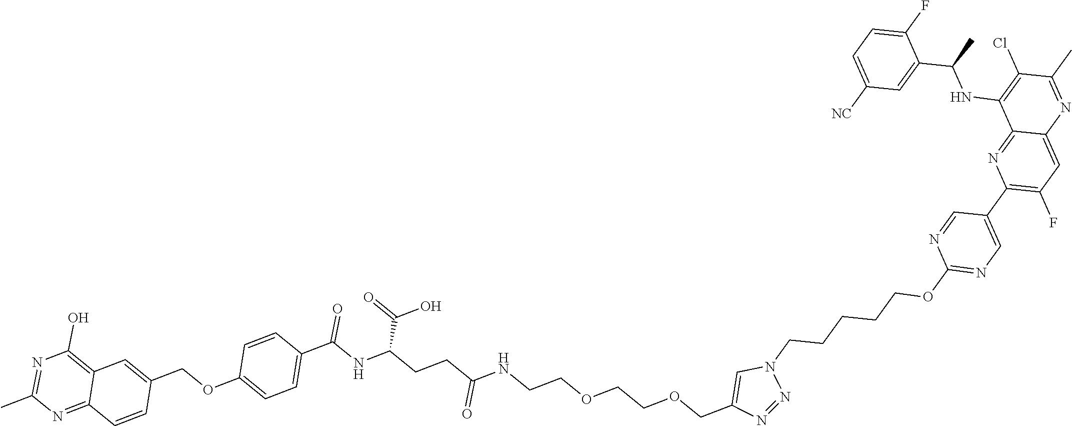 |
| I- 62 | 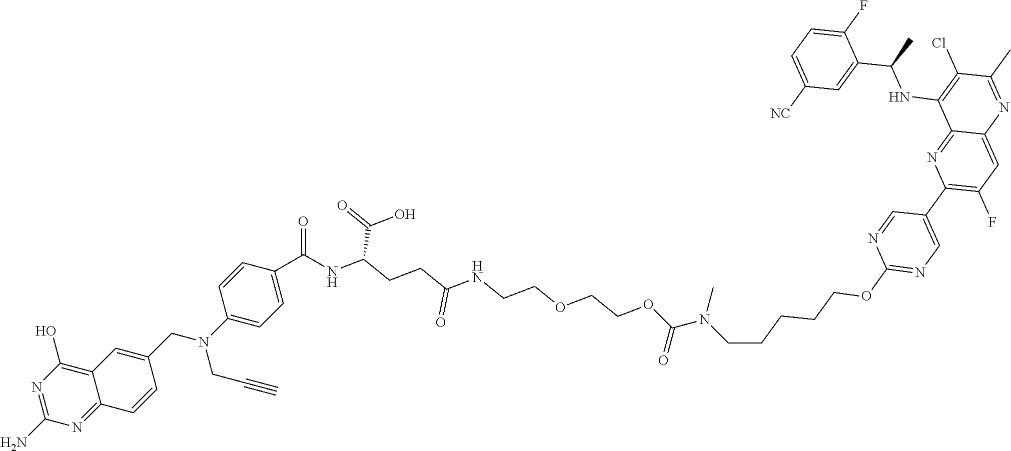 [SMILES: C#CCN(Cc1ccc2nc(N)nc(O)c2c1)c1ccc(C(=O)N[C@@H](CCC(=O)NCCOCCOC(=O)N(C)CCCCCOc2ncc(-c3nc4c(N[C@H](C)c5cc(C#N)ccc5F)c(Cl)c(C)nc4cc3F)cn2)C(=O)O)cc1] |
| I- 63 | 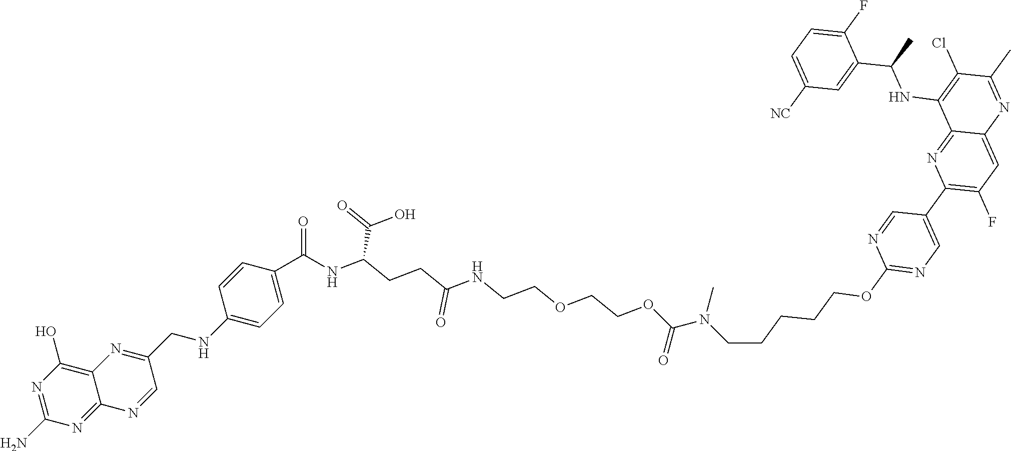 [SMILES: Cc1nc2cc(F)c(-c3cnc(OCCCCCN(C)C(=O)OCCOCCNC(=O)CC[C@H](NC(=O)c4ccc(NCc5cnc6nc(N)nc(O)c6n5)cc4)C(=O)O)nc3)nc2c(N[C@H](C)c2cc(C#N)ccc2F)c1Cl] |
| I- 64 | 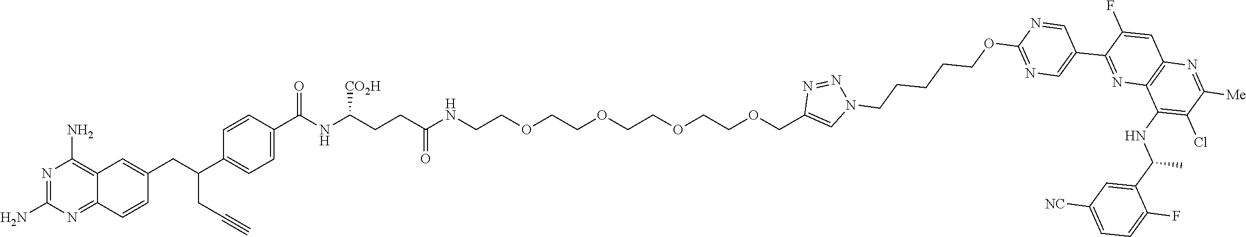 [SMILES: C#CCC(Cc1ccc2nc(N)nc(N)c2c1)c1ccc(C(=O)N[C@@H](CCC(=O)NCCOCCOCCOCCOCc2cn(CCCCCOc3ncc(-c4nc5c(N[C@H](C)c6cc(C#N)ccc6F)c(Cl)c(C)nc5cc4F)cn3)nn2)C(=O)O)cc1] |
| I- 65 | 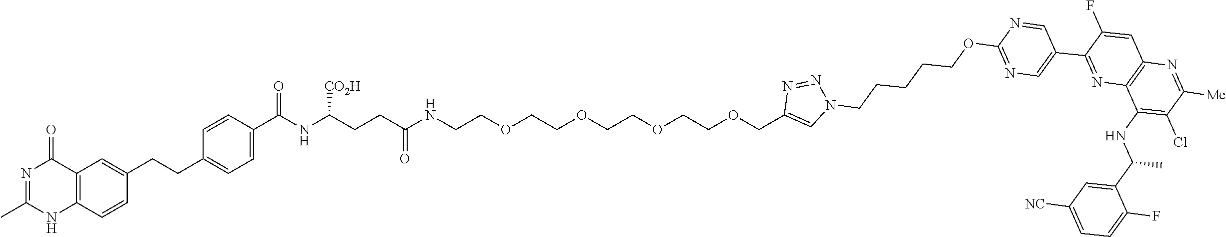 [SMILES: Cc1nc(=O)c2cc(CCc3ccc(C(=O)N[C@@H](CCC(=O)NCCOCCOCCOCCOCc4cn(CCCCCOc5ncc(-c6nc7c(N[C@H](C)c8cc(C#N)ccc8F)c(Cl)c(C)nc7cc6F)cn5)nn4)C(=O)O)cc3)ccc2[nH]1] |
| I- 66 | 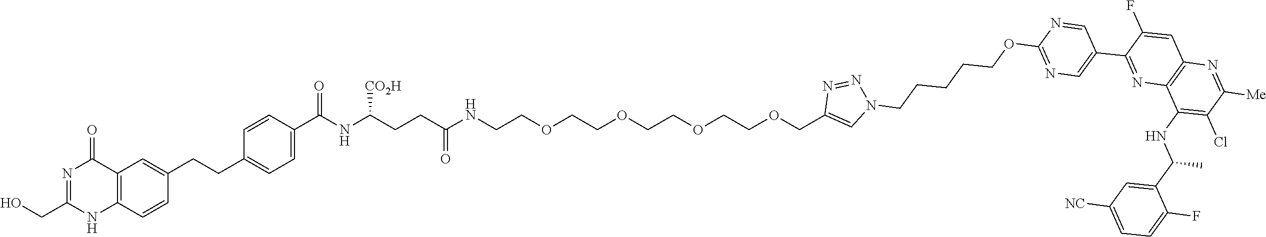 [SMILES: Cc1nc2cc(F)c(-c3cnc(OCCCCCn4cc(COCCOCCOCCOCCNC(=O)CC[C@H](NC(=O)c5ccc(CCc6ccc7[nH]c(CO)nc(=O)c7c6)cc5)C(=O)O)nn4)nc3)nc2c(N[C@H](C)c2cc(C#N)ccc2F)c1Cl] |
| I- 67 | 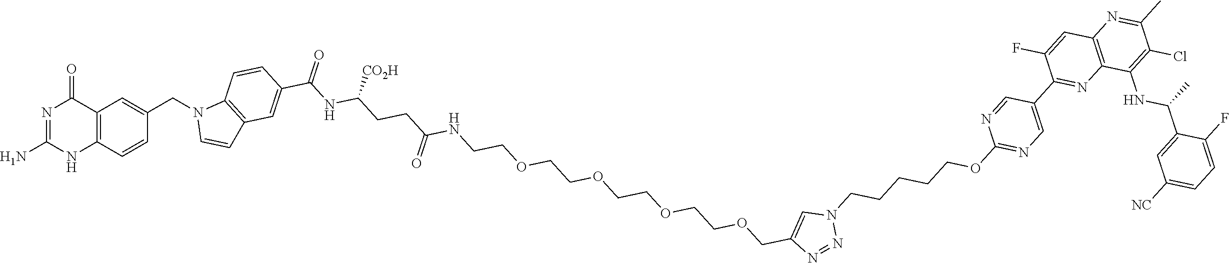 [SMILES: Cc1nc2cc(F)c(-c3cnc(OCCCCCn4cc(COCCOCCOCCOCCNC(=O)CC[C@H](NC(=O)c5ccc6c(ccn6Cc6ccc7[nH]c(N)nc(=O)c7c6)c5)C(=O)O)nn4)nc3)nc2c(N[C@H](C)c2cc(C#N)ccc2F)c1Cl] |
| I- 68 | 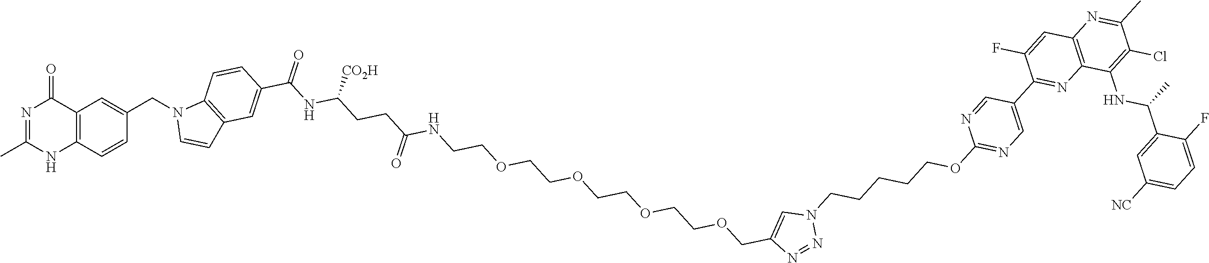 [SMILES: Cc1nc(=O)c2cc(Cn3ccc4cc(C(=O)N[C@@H](CCC(=O)NCCOCCOCCOCCOCc5cn(CCCCCOc6ncc(-c7nc8c(N[C@H](C)c9cc(C#N)ccc9F)c(Cl)c(C)nc8cc7F)cn6)nn5)C(=O)O)ccc43)ccc2[nH]1] |
| I- 69 | 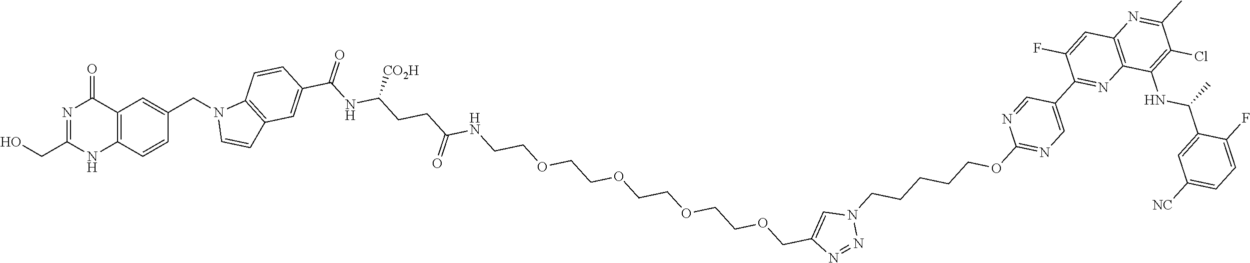 [SMILES: Cc1nc2cc(F)c(-c3cnc(OCCCCCn4cc(COCCOCCOCCOCCNC(=O)CC[C@H](NC(=O)c5ccc6c(ccn6Cc6ccc7[nH]c(CO)nc(=O)c7c6)c5)C(=O)O)nn4)nc3)nc2c(N[C@H](C)c2cc(C#N)ccc2F)c1Cl] |
| I- 70 | 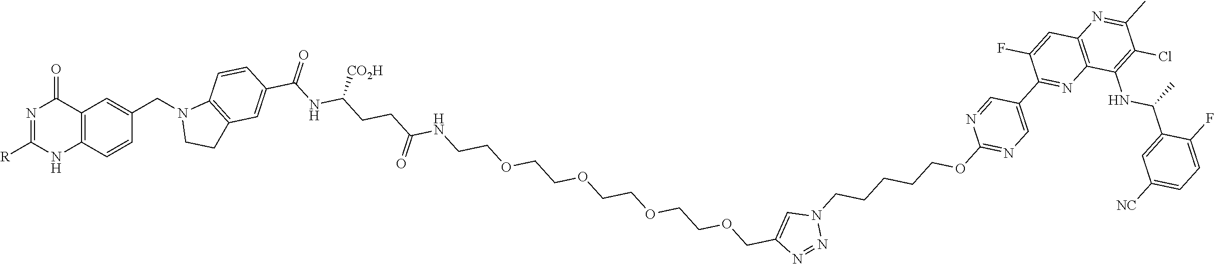 |
| | where R is —NH₂, —Me, or —CH₂—OH (3 separate compounds) |
| I- 71 | 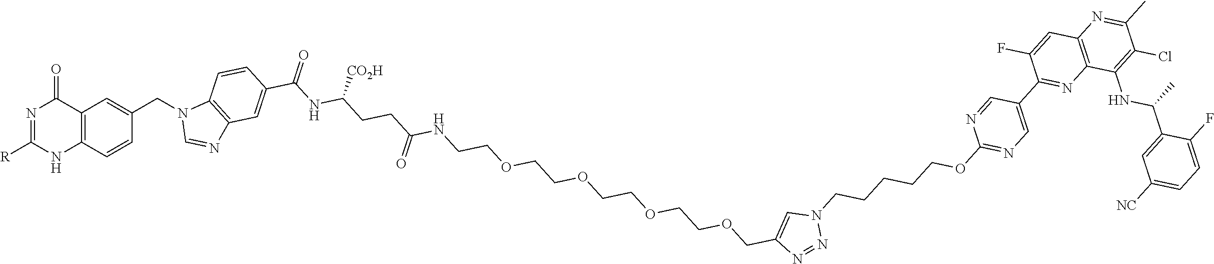 |
| | where R is —NH₂, —Me, or —CH₂—OH (3 separate compounds) |
| I- 72 | 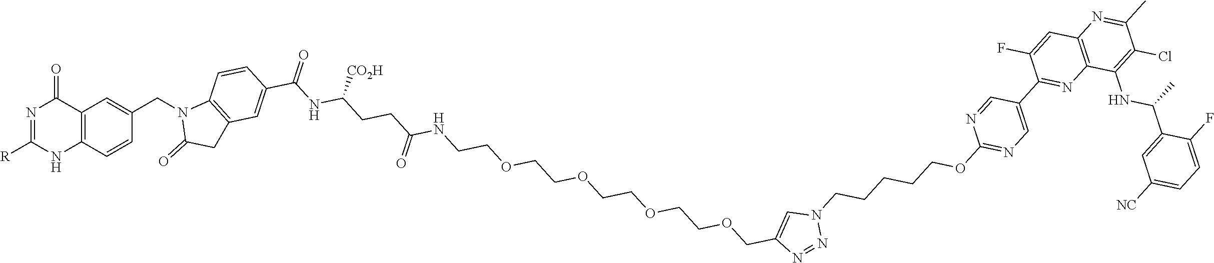 |
| | where R is —NH₂, —Me, or —CH₂—OH (3 separate compounds) |
| I- 73 | 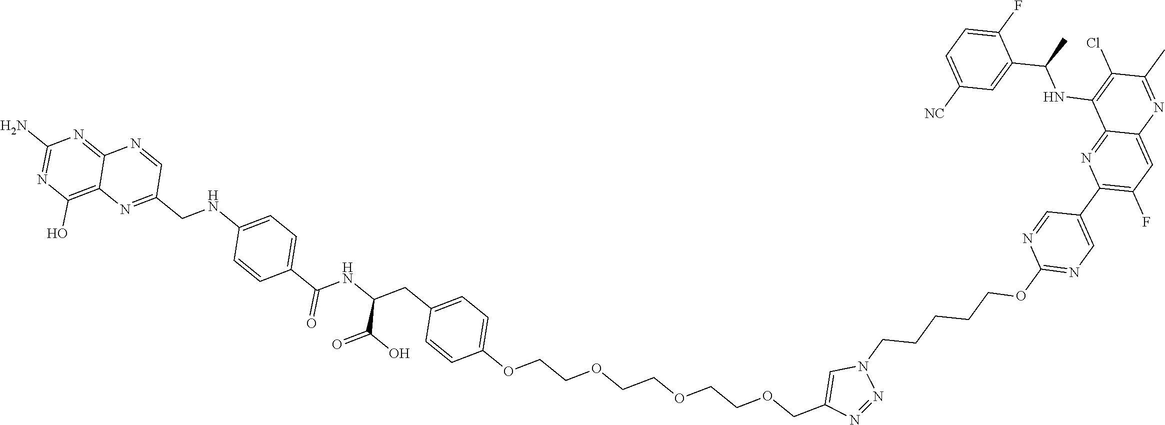 [SMILES: Cc1nc2cc(F)c(-c3cnc(OCCCCCn4cc(COCCOCCOCCOc5ccc(C[C@H](NC(=O)c6ccc(NCc7cnc8nc(N)nc(O)c8n7)cc6)C(=O)O)cc5)nn4)nc3)nc2c(N[C@H](C)c2cc(C#N)ccc2F)c1Cl] |
| I- 78 | 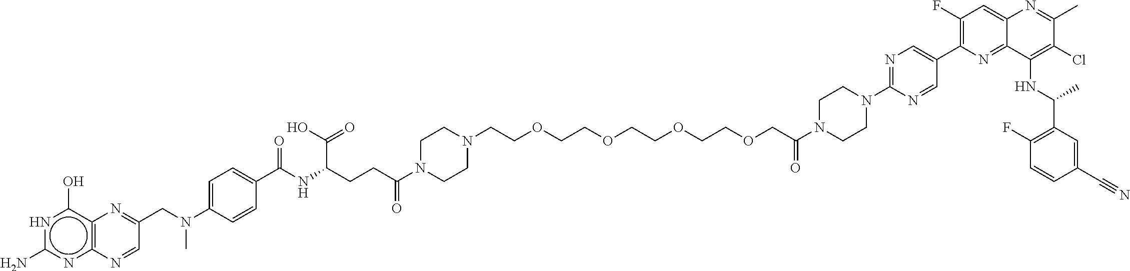 |
| I- 79 | 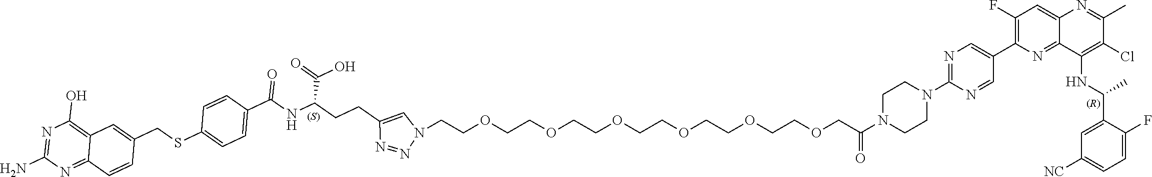 |
| I- 80 | 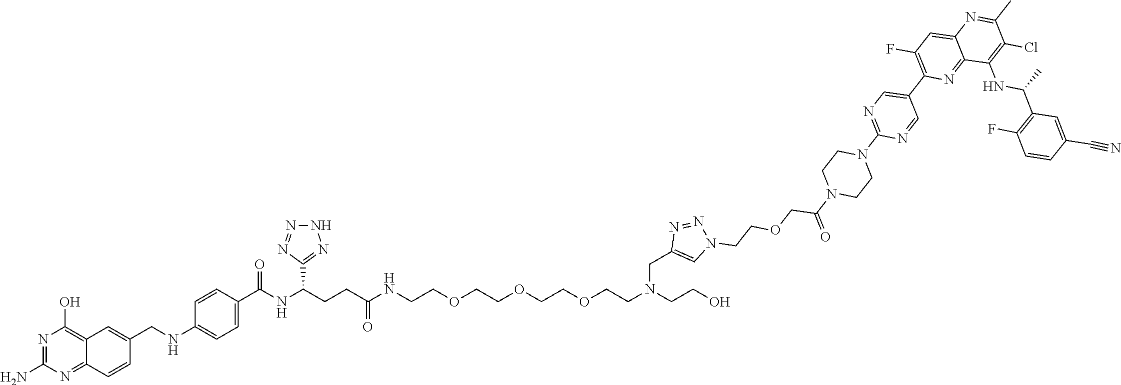 [SMILES: Cc1nc2cc(F)c(-c3cnc(N4CCN(C(=O)COCCn5cc(CN(CCO)CCOCCOCCOCCNC(=O)CC[C@H](NC(=O)c6ccc(NCc7ccc8nc(N)nc(O)c8c7)cc6)c6nn[nH]n6)nn5)CC4)nc3)nc2c(N[C@H](C)c2cc(C#N)ccc2F)c1Cl] |
| I- 81 | 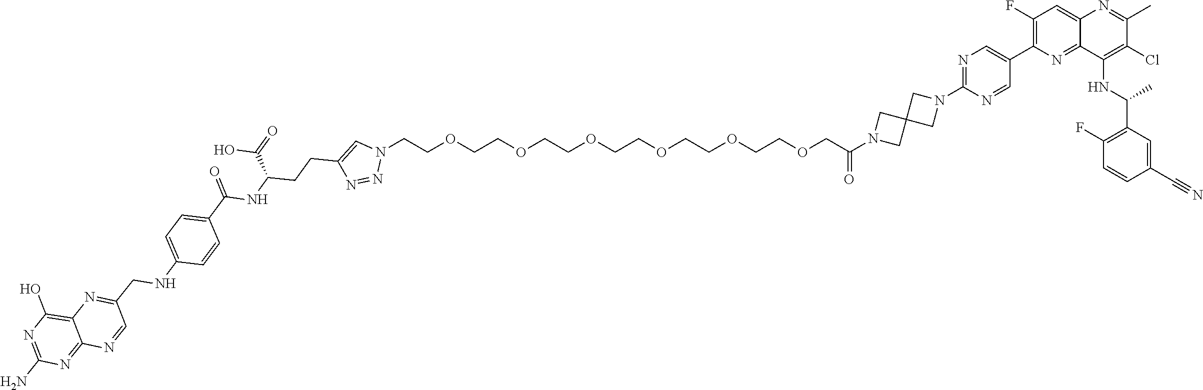 [SMILES: Cc1nc2cc(F)c(-c3cnc(N4CC5(CN(C(=O)COCCOCCOCCOCCOCCOCCn6cc(CC[C@H](NC(=O)c7ccc(NCc8cnc9nc(N)nc(O)c9n8)cc7)C(=O)O)nn6)C5)C4)nc3)nc2c(N[C@H](C)c2cc(C#N)ccc2F)c1Cl] |
| I- 82 | 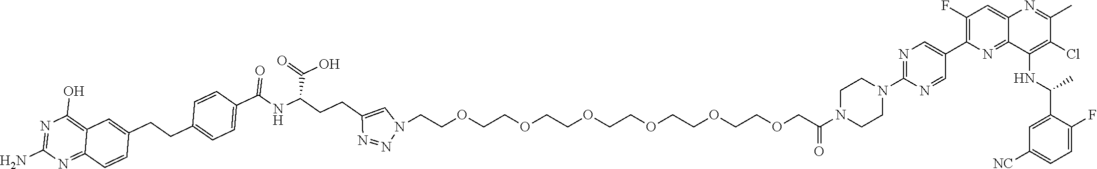 [SMILES: Cc1nc2cc(F)c(-c3cnc(N4CCN(C(=O)COCCOCCOCCOCCOCCOCCn5cc(CC[C@H](NC(=O)c6ccc(CCc7ccc8nc(N)nc(O)c8c7)cc6)C(=O)O)nn5)CC4)nc3)nc2c(N[C@H](C)c2cc(C#N)ccc2F)c1Cl] |
| | ISP2-557 |
| I- 83 | 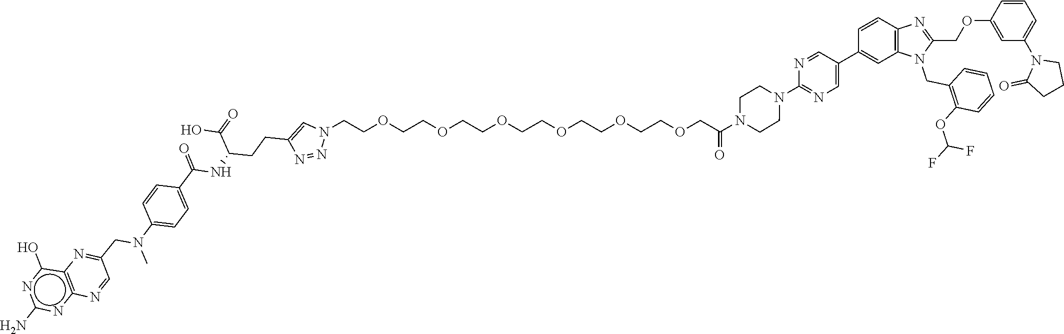 |
| I- 84 | 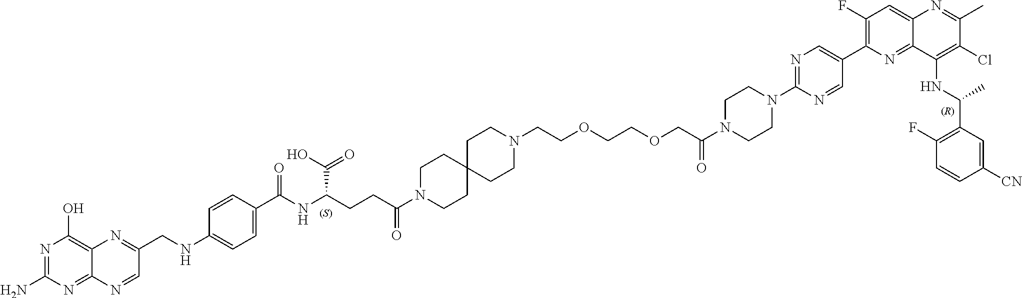 |
| I- 85 | 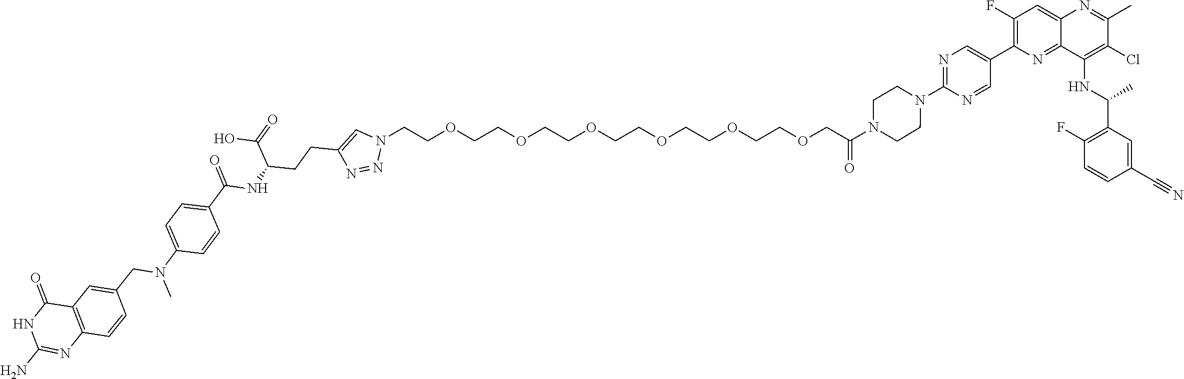 [SMILES: Cc1nc2cc(F)c(-c3cnc(N4CCN(C(=O)COCCOCCOCCOCCOCCOCCn5cc(CC[C@H](NC(=O)c6ccc(N(C)Cc7ccc8nc(N)[nH]c(=O)c8c7)cc6)C(=O)O)nn5)CC4)nc3)nc2c(N[C@H](C)c2cc(C#N)ccc2F)c1Cl] |
| I- 86 | 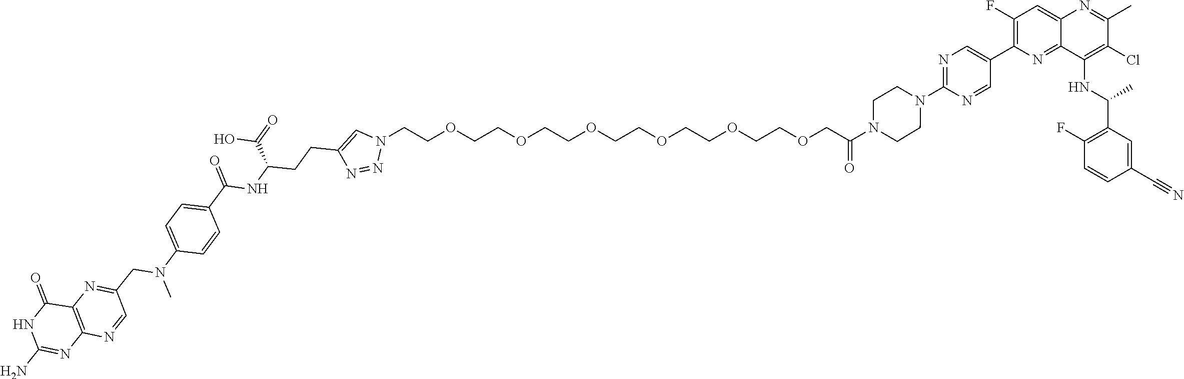 [SMILES: Cc1nc2cc(F)c(-c3cnc(N4CCN(C(=O)COCCOCCOCCOCCOCCOCCn5cc(CC[C@H](NC(=O)c6ccc(N(C)Cc7cnc8nc(N)[nH]c(=O)c8n7)cc6)C(=O)O)nn5)CC4)nc3)nc2c(N[C@H](C)c2cc(C#N)ccc2F)c1Cl] |
| I- 87 | 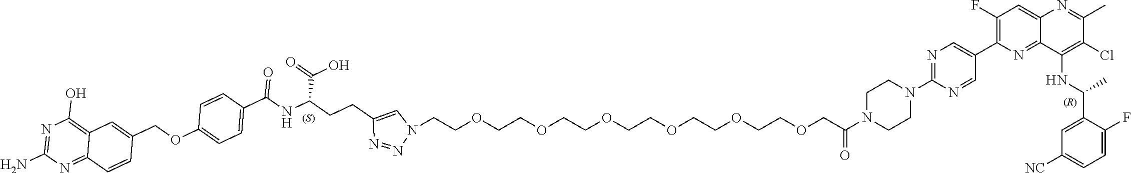 |
| I- 88 | 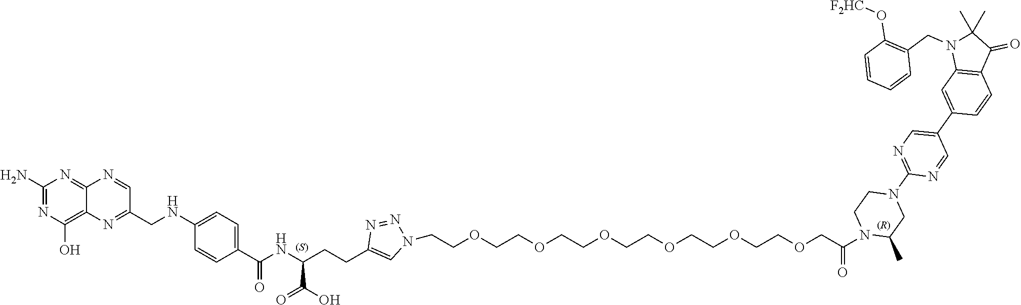 |
| I- 89 | 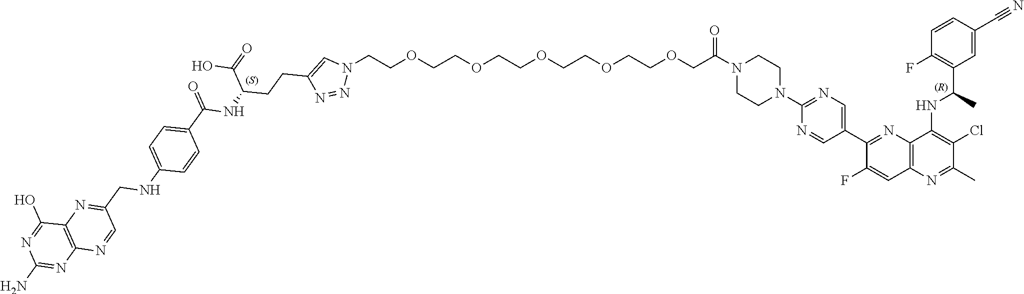 |
| I- 90 | 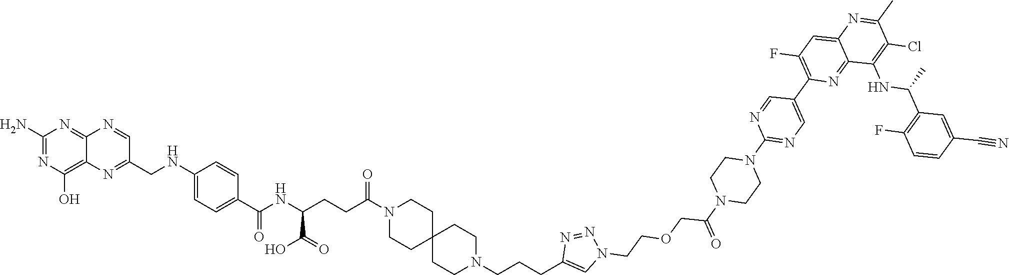 [SMILES: Cc1nc2cc(F)c(-c3cnc(N4CCN(C(=O)COCCn5cc(CCCN6CCC7(CC6)CCN(C(=O)CC[C@H](NC(=O)c6ccc(NCc8cnc9nc(N)nc(O)c9n8)cc6)C(=O)O)CC7)nn5)CC4)nc3)nc2c(N[C@H](C)c2cc(C#N)ccc2F)c1Cl] |
| I- 91 | 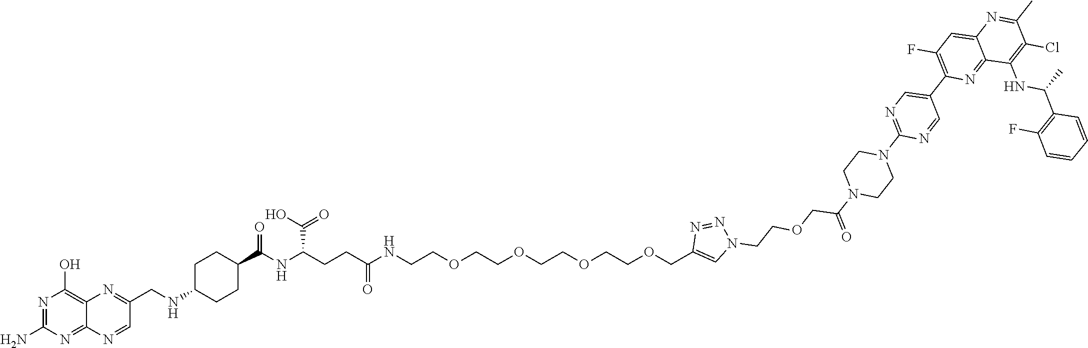 [SMILES: Cc1nc2cc(F)c(-c3cnc(N4CCN(C(=O)COCCn5cc(COCCOCCOCCOCCNC(=O)CC[C@H](NC(=O)[C@H]6CC[C@H](NCc7cnc8nc(N)nc(O)c8n7)CC6)C(=O)O)nn5)CC4)nc3)nc2c(N[C@H](C)c2ccccc2F)c1Cl] |
| I- 92 | 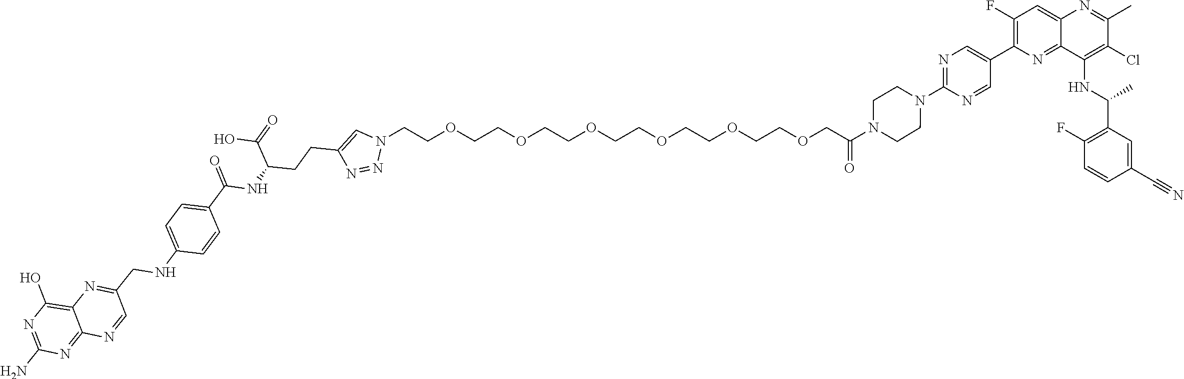 [SMILES: Cc1nc2cc(F)c(-c3cnc(N4CCN(C(=O)COCCOCCOCCOCCOCCOCCn5cc(CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 93 | 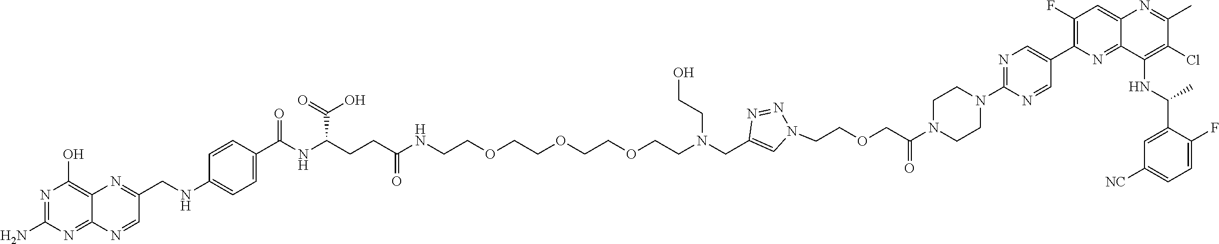 [SMILES: Cc1nc2cc(F)c(-c3cnc(N4CCN(C(=O)COCCn5cc(CN(CCO)CCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 94 | 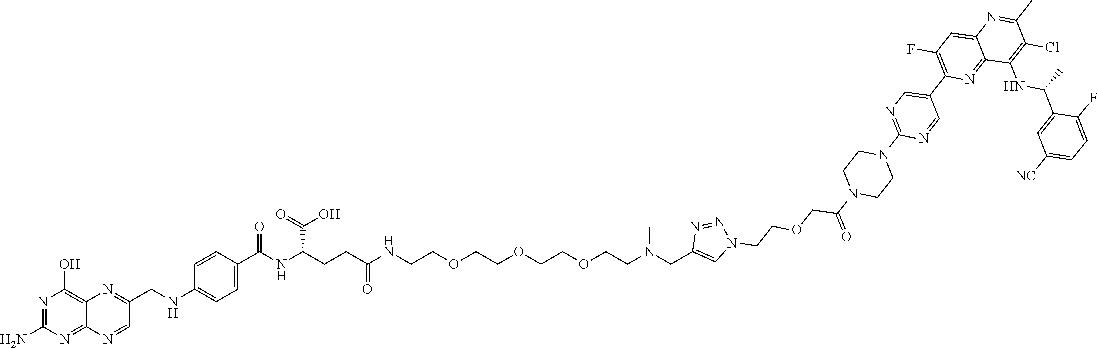 [SMILES: Cc1nc2cc(F)c(-c3cnc(N4CCN(C(=O)COCCn5cc(CN(C)CCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl] |
| I- 95 | 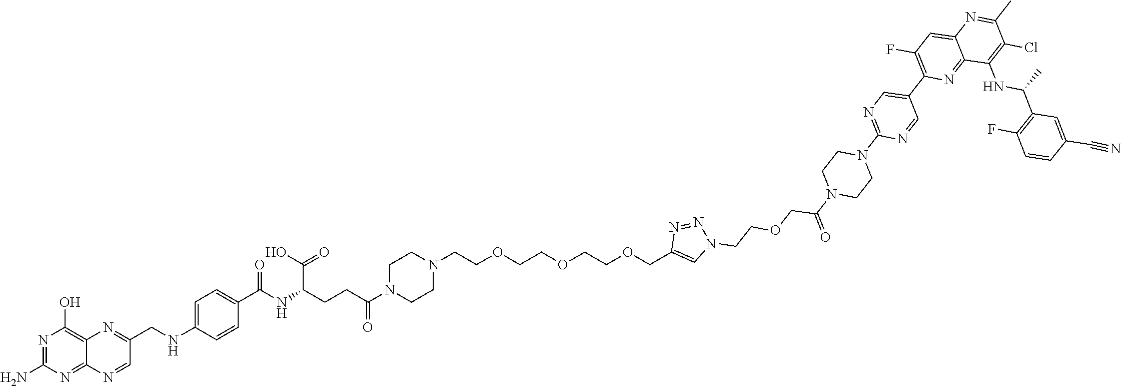 [SMILES: Cc1nc2cc(F)c(-c3cnc(N4CCN(C(=O)COCCn5cc(COCCOCCOCCN6CCN(C(=O)CC[C@H](NC(=O)c7ccc(NCc8cnc9nc(N)nc(O)c9n8)cc7)C(=O)O)CC6)nn5)CC4)nc3)nc2c(N[C@H](C)c2cc(C#N)ccc2F)c1Cl] |

TABLE 7
Exemplary folate receptor binding TNF-alpha targeting compounds of Formula (IIIA)

| # | Compound structure |
|---|---|
| I- 21 | 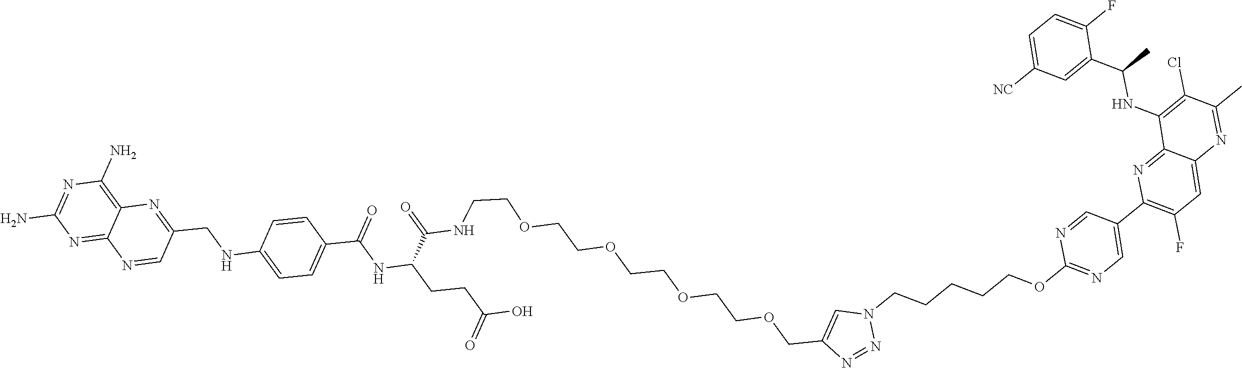 [SMILES: Cc1nc2cc(F)c(-c3cnc(OCCCCCn4cc(COCCOCCOCCOCCNC(=O)[C@H](CCC(=O)O)NC(=O)c5ccc(NCc6cnc7nc(N)nc(N)c7n6)cc5)nn4)nc3)nc2c(N[C@H](C)c2cc(C#N)ccc2F)c1Cl] |
| I- 35 | 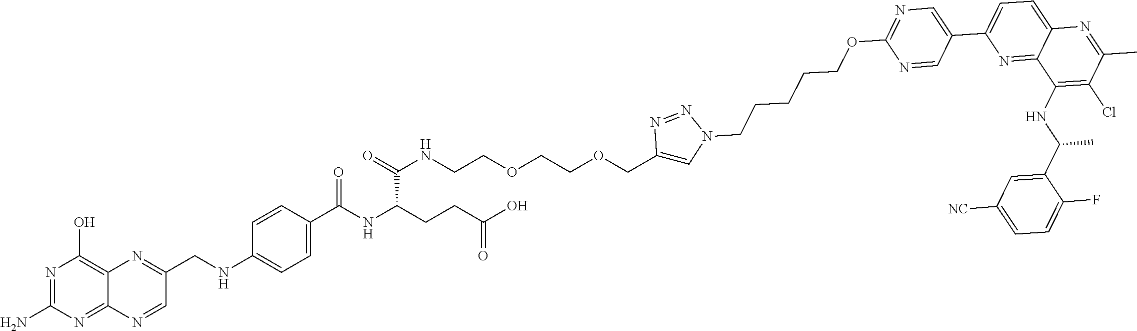 [SMILES: Cc1nc2ccc(-c3cnc(OCCCCCn4cc(COCCOCCNC(=O)[C@H](CCC(=O)O)NC(=O)c5ccc(NCc6cnc7nc(N)nc(O)c7n6)cc5)nn4)nc3)nc2c(N[C@H](C)c2cc(C#N)ccc2F)c1Cl] |
| I- 34 | 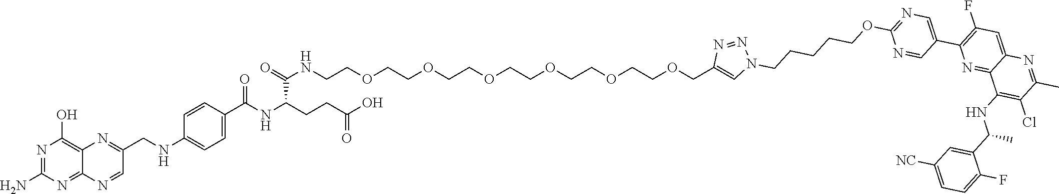 [SMILES: Cc1nc2cc(F)c(-c3cnc(OCCCCCn4cc(COCCOCCOCCOCCOCCOCCNC(=O)[C@H](CCC(=O)O)NC(=O)c5ccc(NCc6cnc7nc(N)nc(O)c7n6)cc5)nn4)nc3)nc2c(N[C@H](C)c2cc(C#N)ccc2F)c1Cl] |
| I- 74 | 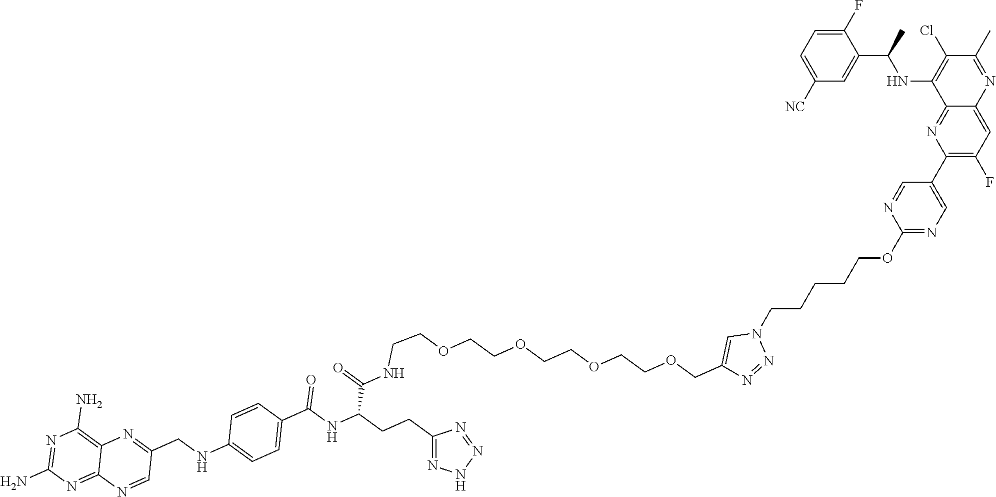 [SMILES: Cc1nc2cc(F)c(-c3cnc(OCCCCCn4cc(COCCOCCOCCOCCNC(=O)[C@H](CCc5nn[nH]n5)NC(=O)c5ccc(NCc6cnc7nc(N)nc(N)c7n6)cc5)nn4)nc3)nc2c(N[C@H](C)c2cc(C#N)ccc2F)c1Cl] |
| I- 97 | 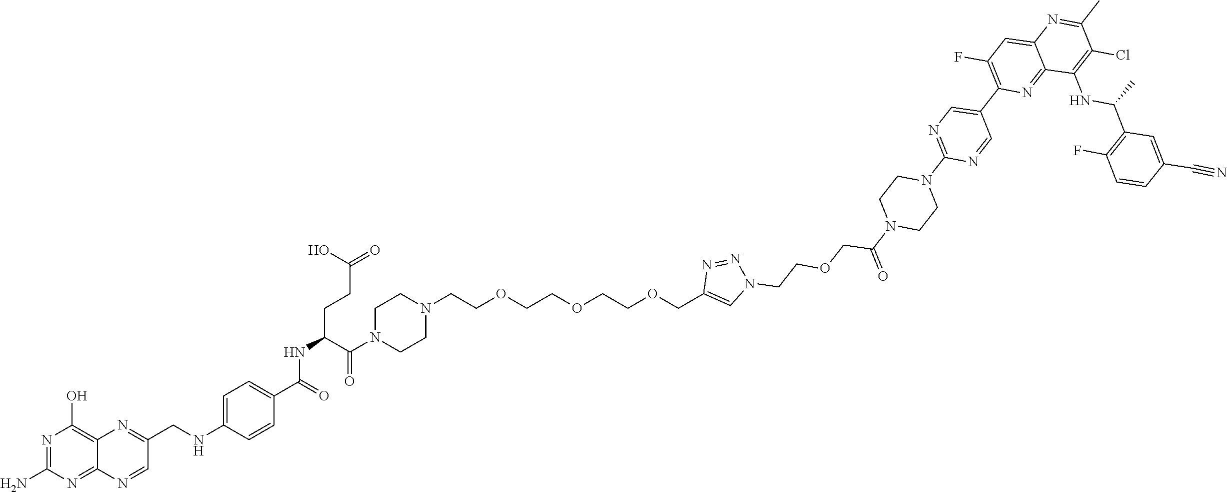 [SMILES: Cc1nc2cc(F)c(-c3cnc(N4CCN(C(=O)COCCn5cc(COCCOCCOCCN6CCN(C(=O)[C@H](CCC(=O)O)NC(=O)c7ccc(NCc8cnc9nc(N)nc(O)c9n8)cc7)CC6)nn5)CC4)nc3)nc2c(N[C@H](C)c2cc(C#N)ccc2F)c1Cl] |

TABLE 8
Exemplary folate receptor binding TNF-alpha targeting compounds of Formula (IIIC)

| # | Compound structure |
|---|---|
| I-96 | 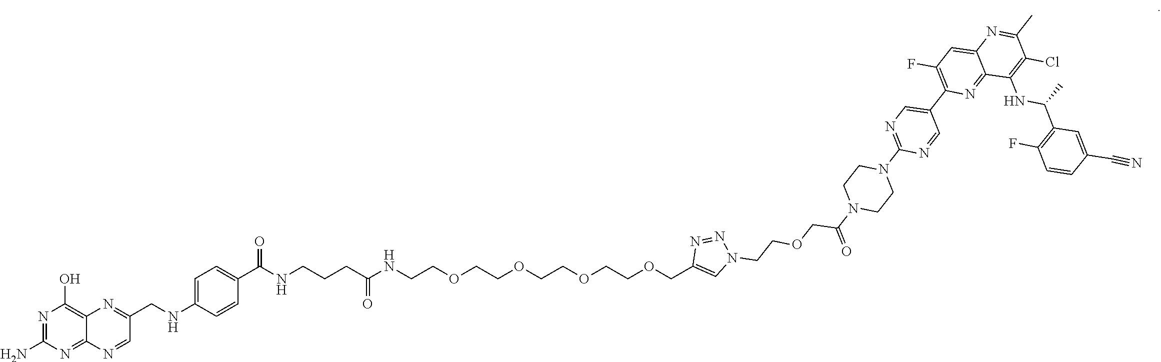 [SMILES: Cc1nc2cc(F)c(-c3cnc(N4CCN(C(=O)COCCn5cc(COCCOCCOCCOCCNC(=O)CCCNC(=O)c6ccc(NCc7cnc8nc(N)nc(O)c8n7)cc6)nn5)CC4)nc3)nc2c(N[C@H](C)c2cc(C#N)ccc2F)c1Cl] |

TABLE 9
Exemplary folate receptor binding TNF-alpha targeting compounds of Formula (IIIB)

| # | Compound structure |
|---|---|
| I-75 | 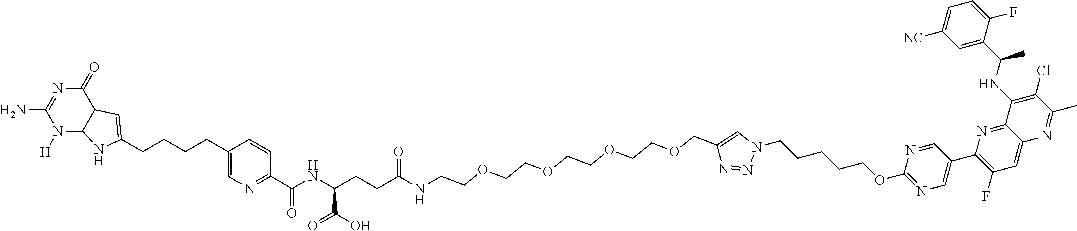 [SMILES: Cc1nc2cc(F)c(-c3cnc(OCCCCCn4cc(COCCOCCOCCOCCNC(=O)CC[C@H](NC(=O)c5ccc(CCCCC6=CC7C(=O)N=C(N)NC7N6)cn5)C(=O)O)nn4)nc3)nc2c(N[C@H](C)c2cc(C#N)ccc2F)c1Cl] |
| I-32 | 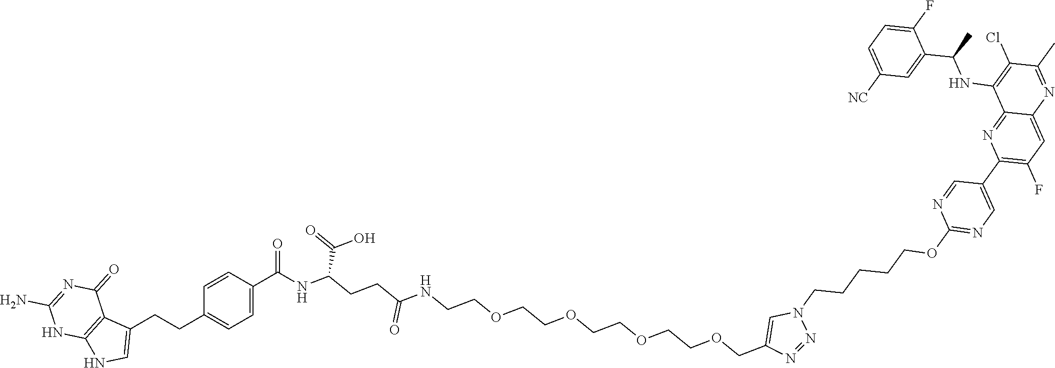 [SMILES: Cc1nc2cc(F)c(-c3cnc(OCCCCCn4cc(COCCOCCOCCOCCNC(=O)CC[C@H](NC(=O)c5ccc(CCc6c[nH]c7[nH]c(N)nc(=O)c67)cc5)C(=O)O)nn4)nc3)nc2c(N[C@H](C)c2cc(C#N)ccc2F)c1Cl] |
| I-98 | 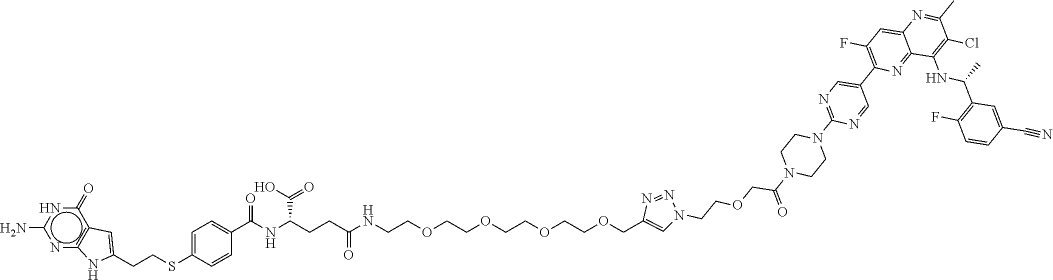 |

TABLE 9
Other example folate receptor binder bifunctional compounds of formula (IIIB)

| # | Compound structure |
|---|---|
| I-37 | 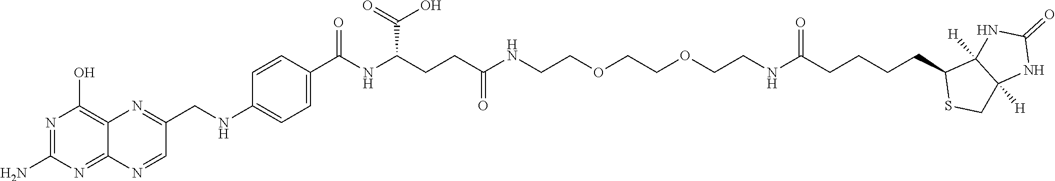 [SMILES: Nc1nc(O)c2nc(CNc3ccc(C(=O)N[C@@H](CCC(=O)NCCOCCOCCNC(=O)CCCC[C@@H]4SC[C@@H]5NC(=O)N[C@@H]54)C(=O)O)cc3)cnc2n1] |
| I-38 | 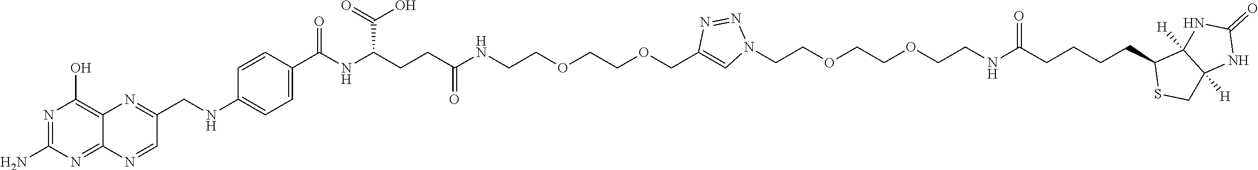 [SMILES: Nc1nc(O)c2nc(CNc3ccc(C(=O)N[C@@H](CCC(=O)NCCOCCOCc4cn(CCOCCOCCNC(=O)CCCC[C@@H]5SC[C@@H]6NC(=O)N[C@@H]65)nn4)C(=O)O)cc3)cnc2n1] |
| I-39 | 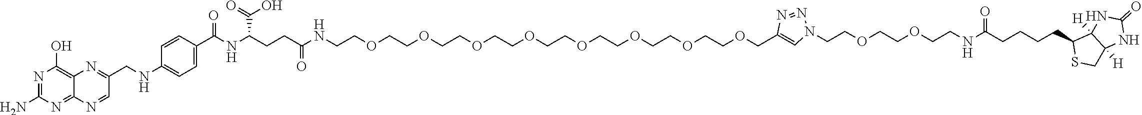 [SMILES: Nc1nc(O)c2nc(CNc3ccc(C(=O)N[C@@H](CCC(=O)NCCOCCOCCOCCOCCOCCOCCOCCOCc4cn(CCOCCOCCNC(=O)CCCC[C@@H]5SC[C@@H]6NC(=O)N[C@@H]65)nn4)C(=O)O)cc3)cnc2n1] |
| I-40 | 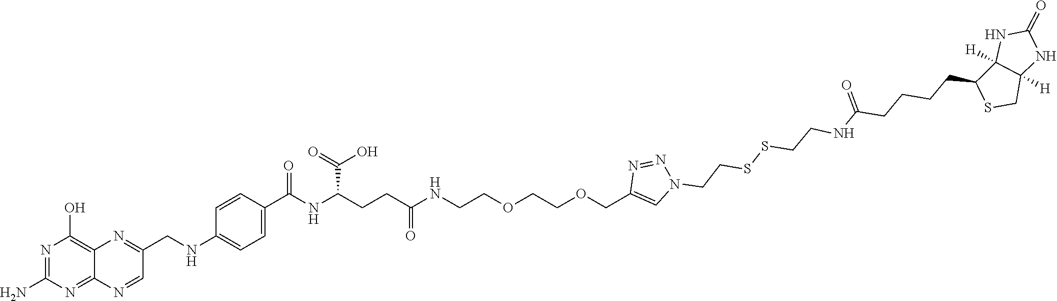 [SMILES: Nc1nc(O)c2nc(CNc3ccc(C(=O)N[C@@H](CCC(=O)NCCOCCOCc4cn(CCSSCCNC(=O)CCCC[C@@H]5SC[C@@H]6NC(=O)N[C@@H]65)nn4)C(=O)O)cc3)cnc2n1] |

### 5.3. Conjugates

The compounds of this disclosure can be referred to as a conjugate, e.g., when the moiety of interest (Y) is a molecule (e.g., as described herein). Such conjugates can be prepared by conjugation of a chemoselective ligation group of any one of the compounds described herein with a compatible reactive group of a molecule Y. The compatible group of the molecule Y can be introduced by modification prior to conjugation, or can be a group present in the molecule. Alternatively, such conjugates can be prepared de novo, e.g., via modification of a Y molecule of interest starting material to introduce a linker, e.g., to which a ligand X can be attached.

Aspects of this disclosure include compounds of formula (I) where the moiety of interest Y is a selected from small molecule, dye, fluorophore, monosaccharide, disaccharide, trisaccharide, and biomolecule.

In some embodiments of the compounds of this disclosure, Y is a biomolecule. In some embodiments, the biomolecule is selected from protein, polynucleotide, polysaccharide, peptide, glycoprotein, lipid, enzyme, antibody, and antibody fragment.

In some embodiments, Y is a molecule that specifically binds to a target molecule, such as an extracellular target protein.

In some embodiments, Y is a molecule that is itself targeted for intracellular delivery.

The compounds of this disclosure can, in some cases, be referred to as a conjugate, e.g., when the moiety of interest (Y) is a molecule such as a biomolecule, where the conjugate can derived from a conjugation or coupling reaction between a chemoselective ligation group and a compatible group on the biomolecule. In some embodiments, the biomolecule is conjugated via a naturally occurring group of the biomolecule. In some embodiments, the biomolecule is conjugated via a compatible functional group that is introduced into the biomolecule prior to chemoselective conjugation. In such cases, the linking moiety between the folate binding moiety (X) and Y incorporates the residual group (e.g., Z) that is the product of the chemoselective ligation chemistry.

### 5.3.1. Target Binding Conjugates

Aspects of this disclosure include compounds of formula (IIIA) or (IIIB) where the moiety of interest Y is a moiety that specifically binds to a target molecule, such as a target protein. The target protein can be the target protein is a membrane bound protein or an extracellular protein. In some embodiments, Y is a small molecule that specifically binds to a target molecule, such as a target protein. In some embodiments of the compounds of this disclosure, Y is a biomolecule that specifically binds to a target protein. This disclosure provides conjugates of the particular folate binding compounds and conjugates. In some embodiments, the conjugate includes a moiety of interest Y that specifically binds a target protein, and can find use in methods of cell uptake or internalization of the target protein via binding to the cell surface receptor, and eventual degradation of the target protein.

In some embodiments, Y is an aptamer that specifically binds to a target molecule, such as a target protein. In some embodiments, Y is a peptide or protein (e.g., peptidic binding motif, protein domain, seered polypeptide, or glycoprotein) that specifically binds to a target molecule, such as a target protein. In some embodiments, Y is an antibody or antibody fragment that specifically binds to a target molecule, such as a target protein. In some embodiments, Y is a polynucleotide or oligonucleotide that specifically binds to a target molecule, such as a target protein or a target nucleic acid.

In some embodiments, one Y biomolecule is conjugated to a single moiety (X) that specifically binds to the cell surface folate receptor via a linker L. In some embodiments, one Y biomolecule is conjugated to one (Xₙ-L)-group, wherein when n=1 the (Xₙ-L)-group is referred to as monovalent, and when n>1 the (Xₙ-L)-group is referred to as multivalent (e.g., bivalent, trivalent, etc.). It is understood that in some embodiments of formula (IIIA) or (IIIB), where Y is a biomolecule, Y can be conjugated to two or more (Xₙ-L)-groups, wherein each (Xₙ-L)-group may itself be monovalent or multivalent (e.g., bivalent, trivalent, etc.). In such cases, the ratio of linked (Xₙ-L)-groups to biomolecule can be referred to as 2 or more.

In some embodiments, Y is an antibody. Accordingly, provided herein are conjugates of the following formula (VIIIa):

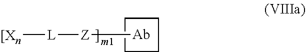

or a pharmaceutically acceptable salt thereof, wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

In some embodiments of formula (VIIIa), L is a linker of formula (IV) (e.g., as described herein). In some embodiments of formula (VIIIa), Xn-L-Z is derived from a compound of formula (I), (IIIA) and (IIIB) (e.g., as described herein), where Y is a chemoselective ligation group.

In some embodiments of formula (VIIIa), L is a linker of formula (IV):

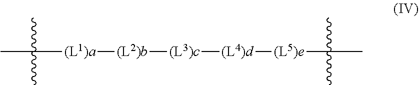

wherein L¹, L², L³, L⁴, L⁵, a, b, c, d, e, and n are defined herein.

In certain embodiments, L is selected from the linkers of Table 3.

In formula (VIIIa), Z can be any convenient residual moiety that results from the covalent linkage or conjugation of a chemoselective ligation group (Y) to a compatible reactive group of an antibody (Ab). In some instances, the compatible reactive group of antibody (Ab) is a group that can naturally be part on the biomolecule. In some instances, the compatible reactive group of antibody (Ab) is one that is introduced or incorporated into the biomolecule prior to conjugation. In such cases, the antibody (Ab) can be a modified version of a biomolecule. For example, a functional group (e.g., an amino group, a carboxylic acid group or a thiol group) of a biomolecule can be modified (e.g., using a chemical reagent such as 2-haloacetyl reagent, or 2-iminothiolane, or the like, or via coupling of a linker group including a chemoselective ligation group, such as an azide, alkyne, or the like) to introduce a compatible chemoselective ligation group.

In some embodiments of formula (VIIIa), Z is selected from the group consisting of

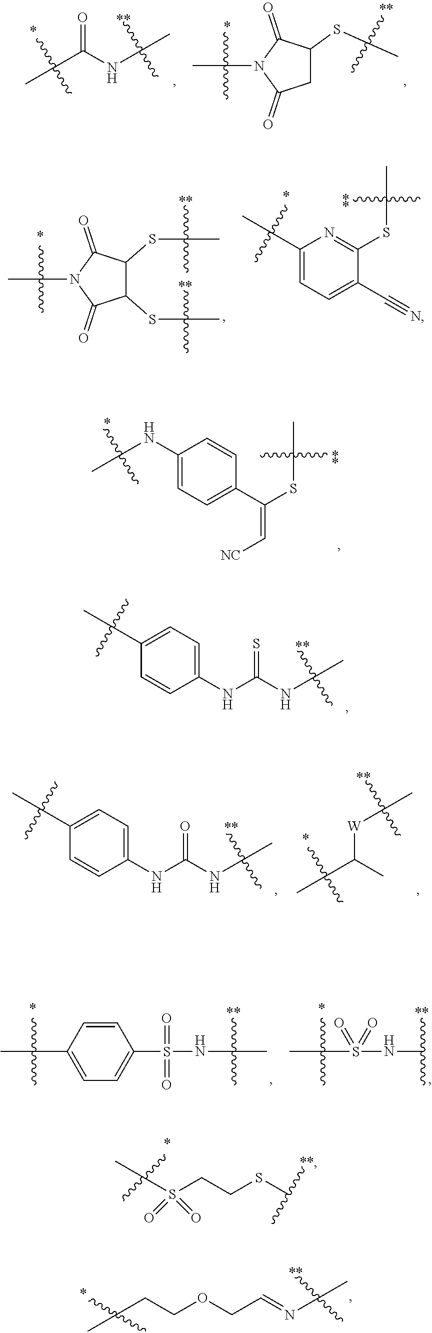

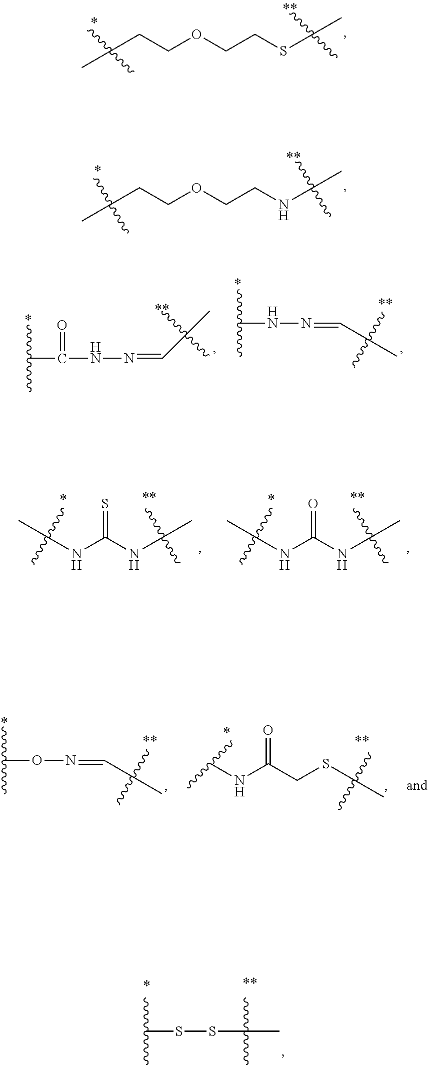

wherein [symbol] represents the point of attachment to the linker L,
wherein [symbol] represents the point of attachment to Ab,

### W is CH₂, N, O or S; and

Ab is an antibody.

In certain embodiments of formula (VIIIa), Z is selected from the group consisting of

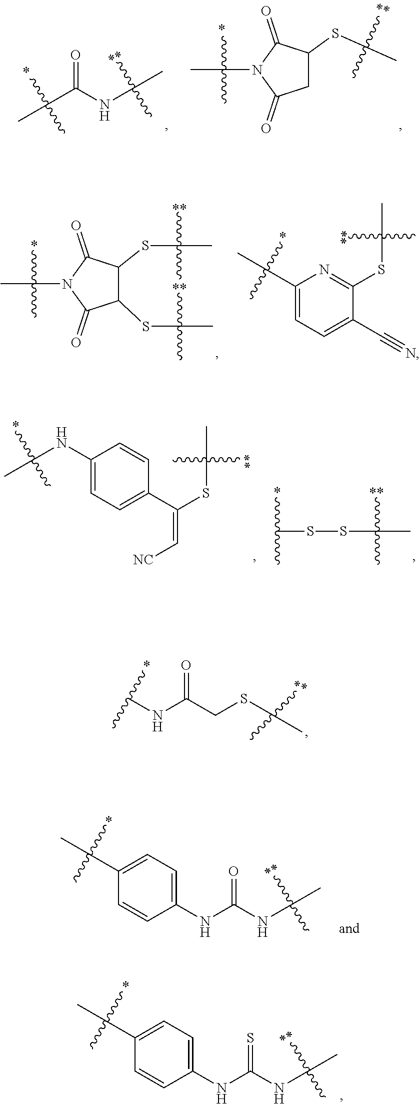

wherein [symbol] represents the point of attachment to L,
wherein [symbol] represents the point of attachment to Ab; and
Ab is an antibody.

In certain embodiments of formula (VIII), Z is selected from the group consisting of

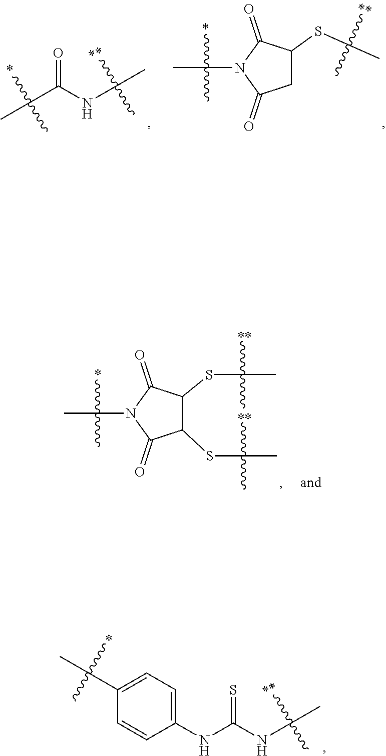

wherein [symbol] represents the point of attachment to L, wherein [symbol] represents the point of attachment to Ab.

In certain embodiments, Z is selected from the moieties of Table 4.

In certain embodiments, n is 1. In certain embodiments, n is 2. In certain embodiments, n is 3. In certain embodiments, n is 4. In certain embodiments, n is 5.

In certain embodiments of formula (VIIIa), n is 1. In certain embodiments, n is 2. In certain embodiments, n is 3. In certain embodiments, n is 4. In certain embodiments, n is 5.

In certain embodiments of formula (VIIIa), the cell surface folate receptor is folate receptor 1 (FRα).

In certain embodiments of formula (VIIIa), the cell surface folate receptor is folate receptor 2 (FRβ).

In certain embodiments of formula (VIIIa), the folate binding moiety X, is of formula (Ia):

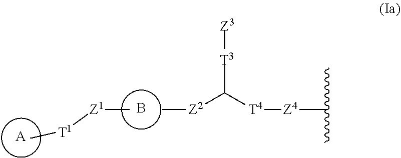

wherein:
A is a ring system of formula (XII):

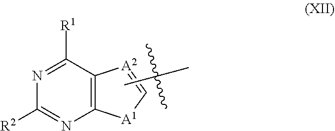

or a tautomer thereof, wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is absent, carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, urea, thiourea, amine, sulfonamide, ether, optionally substituted aryl, optionally substituted heterocycle, and optionally substituted heteroaryl);
each R²¹ is independently selected from H, and optionally substituted (C₁-C₆)alkyl;
and [symbol] represents the point of attachment to -L-Y (e.g., as described herein).

In certain embodiments of formula (VIIIa), X is not folic acid, methotrexate, or pemetrexed.

In certain embodiments of formula (VIIIa), each X is independently of formula (Va), (Vb), (Vc), (Vd), (Ve), or (Vf):

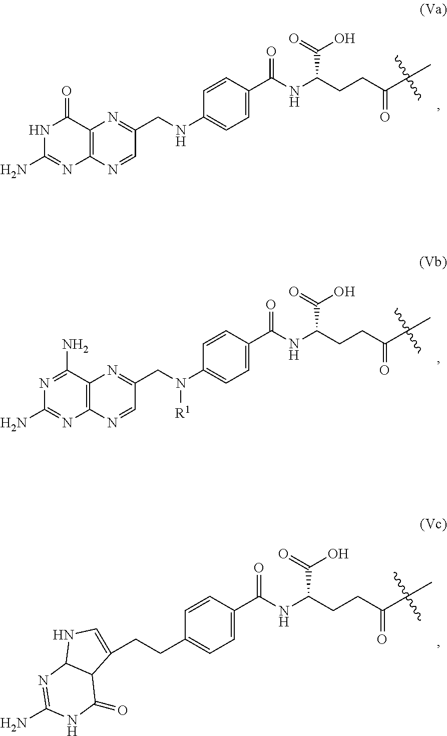

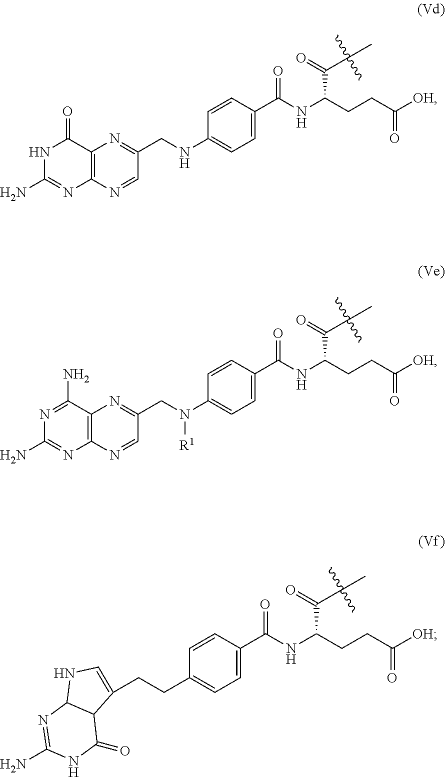

wherein R¹ is —H or —CH₃.

In certain embodiments of formula (VIIIa), each X is independently of formula (Vg)-(Vn):

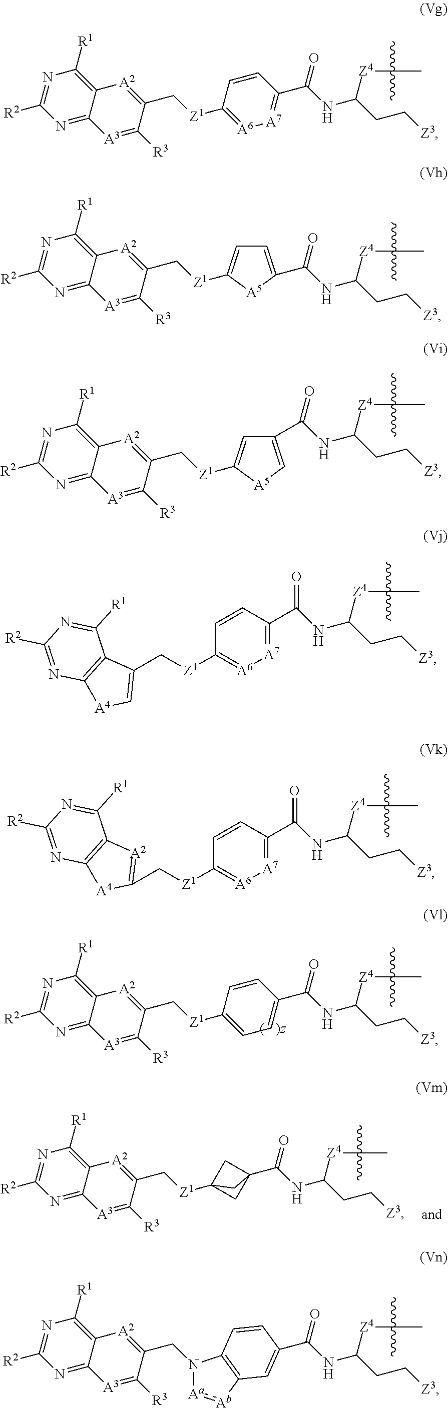

wherein R¹-R³, A²-A⁷, A^{a}-A^{b}, Z¹, Z³-Z⁴ and z are as described herein.

In certain embodiments of formula (VIIIa), each X is independently of formula (VI), (Vm), (Vn), (Vo), or (Vp):

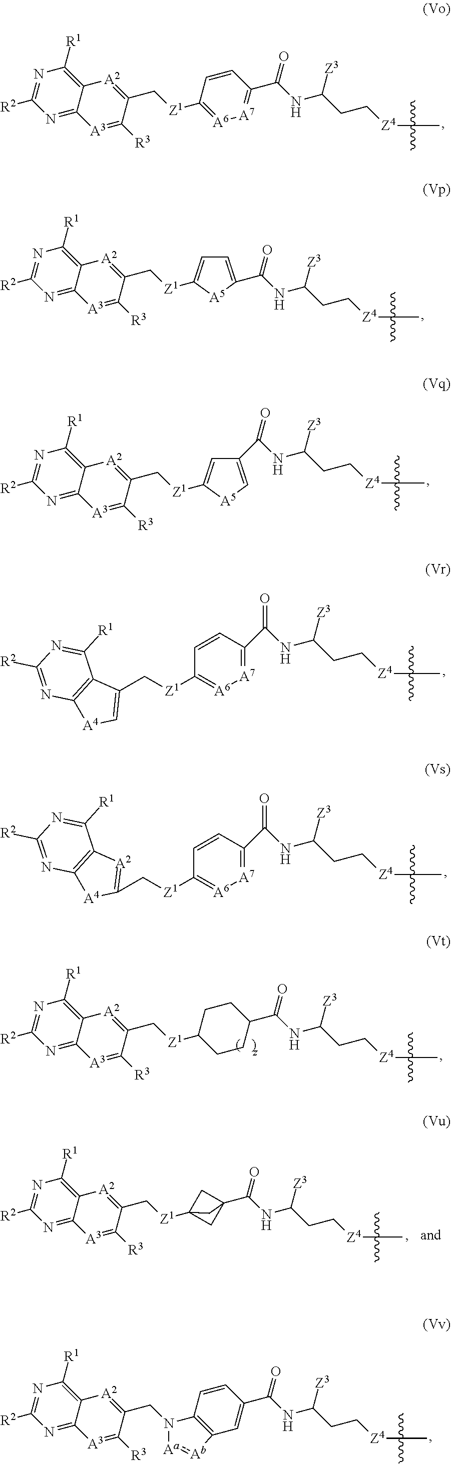

wherein R¹-R³, A²-A⁷, A^{a}-A^{b}, Z¹, Z³-Z⁴ and z are as described herein.

In certain embodiments of formula (VIIIa), each X is independently selected from a compound of Tables 2-3.

In certain embodiments of formula (VIIIa), n is 1 to 6, such as 1 to 5, 1 to 4, 1 to 3, or 1 to 2. In certain cases, n is 2 or less. In certain embodiments, n is 1. In certain embodiments, n is at least 2. In certain instances, n is 2. In certain embodiments, n is 3. In certain embodiments, n is 4. In certain embodiments, n is 5.

In certain embodiments of formula (VIIIa), m1 is 1 to 20, such as 1 to 18, 1 to 16, 1 to 14, 1 to 12, 1 to 10, 1 to 8, 1 to 6, or 1 to 4. In certain instances, m1 is 1 to 12, such as 1 to 10, 1 to 8, 1 to 6, or 1 to 4. In certain instances, m1 is at least about 2. In certain cases, m1 is at least about 3. In certain cases, m1 is at least about 4.

In certain embodiments of formula (VIIIa), Z is a residual moiety resulting from the covalent linkage of a thiol-reactive chemoselective ligation group to one or more cysteine residue(s) of Ab.

In certain embodiments of formula (VIIIa), Z is a residual moiety resulting from the covalent linkage of an amine-reactive chemoselective ligation group to one or more lysine residue(s) of Ab.

In certain embodiments, the conjugates with their linker structures described herein have weaker binding affinity to cell surface receptors. Without being bound to any particular mechanism or theory, such weaker binding affinity may be corrected to longer half-life of the conjugates, and may be useful for tuning (e.g., modifying) the pharmacokinetic properties of the conjugates described herein. In certain embodiments, such weaker binding conjugates still have sufficiently robust uptake.

The term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

The term “pharmaceutically acceptable salt” refers to those salts of the conjugate provided herein, which are formed by the process of the present application which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the conjugate compounds, or separately by reacting the free base function or group of a compound with a suitable organic acid. Examples of pharmaceutically acceptable salts include, but are not limited to, nontoxic acid addition salts, or salts of an amino group formed with inorganic acids such as hydrochloric acid, hydrobromic acid, phosphoric acid, etc., or with organic acids such as acetic acid, maleic acid, tartaric acid, citric acid, succinic acid or malonic acid. Other pharmaceutically acceptable salts include, but are not limited to, adipate, alginate, ascorbate, benzenesulfonate, benzoate, bisulfate, citrate, digluconate, dodecylsulfate, ethanesulfonate, formate, fumarate, gluconate, 2-hydroxy-ethanesulfonate, lactate, laurate, malate, maleate, malonate, methanesulfonate, oleate, oxalate, palmitate, phosphate, propionate, stearate, succinate, sulfate, tartrate, p-toluenesulfonate, valerate salts, and the like. Representative alkali or alkaline earth metal salts include sodium, lithium, potassium, calcium, or magnesium salts, and the like. Further pharmaceutically acceptable salts include, nontoxic ammonium, quaternary ammonium, and amine cations formed using counterions such as halide, hydroxide, carboxylate, sulfate, phosphate, nitrate, alkyl groups having from 1 to 6 carbon atoms (e.g., C₁₋₆ alkyl), sulfonate and aryl sulfonate.

Conjugates of the polypeptide (P), e.g., an antibody (Ab) and compound (Xn-L-Y) may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In certain embodiments of the conjugates described herein, L is bonded through an amide bond to a lysine residue of P. In certain embodiments of the conjugates described herein, L is bonded through a thioether bond to a cysteine residue of P. In certain embodiments of the conjugates described herein, L is bonded through an amide bond to a lysine residue of Ab, as depicted above. In certain embodiments of the conjugates described herein, L is bonded through a thioether bond to a cysteine residue of Ab, as depicted above. In certain embodiments of the conjugates described herein, L is bonded through two thioether bonds to two cysteine residues of Ab, wherein the two cysteine residues are from an opened cysteine-cysteine disulfide bond in Ab, as depicted above. In certain embodiments, the opened cysteine-cysteine disulfide bond is an interchain disulfide bond.

In certain embodiments of the conjugates described herein, when L is bonded through an amide bond to a lysine residue of P, m is an integer from 1 to 80. In certain embodiments of the conjugates described herein, when L is bonded through a thioether bond to a cysteine residue of P, m is an integer from 1 to 8.

In certain embodiments, conjugation to the polypeptide P or the antibody Ab may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide or antibody folding and assembly or alter polypeptide or antigen binding (see, e.g., Junutula et al., J. Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety. All such methodologies are contemplated for use in connection with making the conjugates described herein.

Loading of the compounds of formula (I) to the polypeptides (e.g., antibodies) described herein is represented by “ml” in formula (VIIIa), and is the average number of units of “Xn-L-” or “Xn-” per conjugate molecule. As used herein, the term “DAR” refers to the average value of “m” or the loading of the conjugate. The number of “X” moieties (e.g., folate moieties) per each unit of “Xn-L-” or “Xn-” is represented by “n” in formula (IIIa). As used herein, the term “valency” or “valencies” refers to the number of “X” moieties per unit (“n”). It will be understood that loading, or DAR, is not necessarily equivalent to the number of “X” moieties per conjugate molecule. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and four “Xn-L-” units per conjugate (m=4), there will be 2×4=8 “X” moieties per conjugate. Accordingly, for the conjugates described herein, the total number of “X” moieties per conjugate molecule will be n×m. As used herein, the term “total valency” or “total valencies” refers to the total number of “X” moieties per conjugate molecule (n×m; total valency).

DAR (loading) may range from 1 to 80 units per conjugate. The conjugates provided herein may include collections of polypeptides, antibodies or antigen binding fragments conjugated with a range of units, e.g., from 1 to 80. The average number of units per polypeptide or antibody in preparations of the conjugate from conjugation reactions may be characterized by conventional means such as mass spectroscopy. The quantitative distribution of DAR (loading) in terms of m may also be determined. In some instances, separation, purification, and characterization of homogeneous conjugate where m is a certain value may be achieved by means such as electrophoresis.

In certain embodiments, the DAR for a conjugate provided herein ranges from 1 to 80. In certain embodiments, the DAR for a conjugate provided herein ranges from 1 to 70. In certain embodiments, the DAR for a conjugate provided herein ranges from 1 to 60. In certain embodiments, the DAR for a conjugate provided herein ranges from 1 to 50. In certain embodiments, the DAR for a conjugate provided herein ranges from 1 to 40. In certain embodiments, the DAR for a conjugate provided herein ranges from 1 to 35. In certain embodiments, the DAR for a conjugate provided herein ranges from 1 to 30. In certain embodiments, the DAR for a conjugate provided herein ranges from 1 to 25. In certain embodiments, the DAR for a conjugate provided herein ranges from 1 to 20. In certain embodiments, the DAR for a conjugate provided herein ranges from 1 to 18. In certain embodiments, the DAR for a conjugate provided herein ranges from 1 to 15. In certain embodiments, the DAR for a conjugate provided herein ranges from 1 to 12. In certain embodiments, the DAR for a conjugate provided herein ranges from 1 to 10. In certain embodiments, the DAR for a conjugate provided herein ranges from 1 to 9. In certain embodiments, the DAR for a conjugate provided herein ranges from 1 to 8. In certain embodiments, the DAR for a conjugate provided herein ranges from 1 to 7. In certain embodiments, the DAR for a conjugate provided herein ranges from 1 to 6. In certain embodiments, the DAR for a conjugate provided herein ranges from 1 to 5. In certain embodiments, the DAR for a conjugate provided herein ranges from 1 to 4. In certain embodiments, the DAR for a conjugate provided herein ranges from 1 to 3. In certain embodiments, the DAR for a conjugate provided herein ranges from 2 to 12. In certain embodiments, the DAR for a conjugate provided herein ranges from 2 to 10. In certain embodiments, the DAR for a conjugate provided herein ranges from 2 to 9. In certain embodiments, the DAR for a conjugate provided herein ranges from 2 to 8. In certain embodiments, the DAR for a conjugate provided herein ranges from 2 to 7. In certain embodiments, the DAR for a conjugate provided herein ranges from 2 to 6. In certain embodiments, the DAR for a conjugate provided herein ranges from 2 to 5. In certain embodiments, the DAR for a conjugate provided herein ranges from 2 to 4. In certain embodiments, the DAR for a conjugate provided herein ranges from 3 to 12. In certain embodiments, the DAR for a conjugate provided herein ranges from 3 to 10. In certain embodiments, the DAR for a conjugate provided herein ranges from 3 to 9. In certain embodiments, the DAR for a conjugate provided herein ranges from 3 to 8. In certain embodiments, the DAR for a conjugate provided herein ranges from 3 to 7. In certain embodiments, the DAR for a conjugate provided herein ranges from 3 to 6. In certain embodiments, the DAR for a conjugate provided herein ranges from 3 to 5. In certain embodiments, the DAR for a conjugate provided herein ranges from 3 to 4.

In certain embodiments, the DAR for a conjugate provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the DAR for a conjugate provided herein is about 1, about 2, about 3, about 4, about 5, about 6, about 7, about 8, about 9, about 10, about 11, about 12, or more. In some embodiments, the DAR for a conjugate provided herein is about 3.1, about 3.2, about 3.3, about 3.4, about 3.5, about 3.6, about 3.7, about 3.8, or about 3.9.

In some embodiments, the DAR for a conjugate provided herein ranges from 2 to 20, 2 to 19, 2 to 18, 2 to 17, 2 to 16, 2 to 15, 2 to 14, or 2 to 13. In some embodiments, the DAR for a conjugate provided herein ranges from 3 to 20, 3 to 19, 3 to 18, 3 to 17, 3 to 16, 3 to 15, 3 to 14, or 3 to 13. In some embodiments, the DAR for a conjugate provided herein is about 1. In some embodiments, the DAR for a conjugate provided herein is about 2. In some embodiments, the DAR for a conjugate provided herein is about 3. In some embodiments, the DAR for a conjugate provided herein is about 4. In some embodiments, the DAR for a conjugate provided herein is about 3.8. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8. In some embodiments, the DAR for a conjugate provided herein is about 9. In some embodiments, the DAR for a conjugate provided herein is about 10. In some embodiments, the DAR for a conjugate provided herein is about 11. In some embodiments, the DAR for a conjugate provided herein is about 12. In some embodiments, the DAR for a conjugate provided herein is about 13. In some embodiments, the DAR for a conjugate provided herein is about 14. In some embodiments, the DAR for a conjugate provided herein is about 15. In some embodiments, the DAR for a conjugate provided herein is about 16. In some embodiments, the DAR for a conjugate provided herein is about 17. In some embodiments, the DAR for a conjugate provided herein is about 18. In some embodiments, the DAR for a conjugate provided herein is about 19. In some embodiments, the DAR for a conjugate provided herein is about 20.

In some embodiments, the DAR for a conjugate provided herein is about 25. In some embodiments, the DAR for a conjugate provided herein is about 30. In some embodiments, the DAR for a conjugate provided herein is about 35. In some embodiments, the DAR for a conjugate provided herein is about 40. In some embodiments, the DAR for a conjugate provided herein is about 50. In some embodiments, the DAR for a conjugate provided herein is about 60. In some embodiments, the DAR for a conjugate provided herein is about 70. In some embodiments, the DAR for a conjugate provided herein is about 80.

In certain embodiments, fewer than the theoretical maximum of units are conjugated to the polypeptide, e.g., antibody, during a conjugation reaction. A polypeptide may contain, for example, lysine residues that do not react with the compound or linker reagent. Generally, for example, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the compound is conjugated via a lysine residue on the antibody. In some embodiments, the linker unit or a drug unit is conjugated via a cysteine residue on the antibody.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

The DAR (loading) of a conjugate may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the polypeptide, such that the number and position of cysteine residues is modified for control of the number and/or position of linker-drug attachments (such as for thiomabs prepared as disclosed in WO2006/034488 (herein incorporated by reference in its entirety)).

It is to be understood that the preparation of the conjugates described herein may result in a mixture of conjugates with a distribution of one or more units attached to a polypeptide, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

### 5.4. Pharmaceutical Compositions

In another embodiment, provided herein are pharmaceutical compositions comprising one or more conjugates disclosed herein and a pharmaceutically acceptable carrier.

In certain embodiments, the pharmaceutical compositions provided herein contain therapeutically effective amounts of one or more of the conjugates provided herein, and optionally one or more additional prophylactic or therapeutic agents, in a pharmaceutically acceptable carrier.

Pharmaceutical carriers suitable for administration of the conjugates provided herein include any such carriers known to those skilled in the art to be suitable for the particular mode of administration.

The conjugates described herein can be formulated as the sole pharmaceutically active ingredient in the composition or can be combined with other active ingredients.

In certain embodiments, the conjugate is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, a conjugate described herein may be mixed with a suitable pharmaceutical carrier. The concentration of the conjugate in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of conjugate is dissolved, suspended, dispersed or otherwise mixed in a selected carrier at an effective concentration such that the treated condition is relieved, prevented, or one or more symptoms are ameliorated.

Concentrations of the conjugate in a pharmaceutical composition provided herein will depend on, e.g., the physicochemical characteristics of the conjugate, the dosage schedule, and amount administered as well as other factors known to those of skill in the art.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a conjugate sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the conjugates herein are in a liquid pharmaceutical formulation. Liquid pharmaceutically administrable formulations can, for example, be prepared by dissolving, dispersing, or otherwise mixing a conjugate and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 22nd ed., Pharmaceutical Press, Philadelphia, PA Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a conjugate described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a conjugate provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the conjugates provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

### 5.5. Uses and Methods

### 5.5.1. Uses of Conjugates

In one aspect, provided herein are methods of using the conjugates described herein to remove a polypeptide of interest (a target protein) from a cell's surface. In one aspect, provided herein are methods of using the conjugates described herein to remove a polypeptide of interest (a target protein) from the extracellular milieu. For example, in one embodiment, provided herein are methods of using the conjugates described herein to remove a polypeptide of interest (a target protein) from the surface of a cell by sequestering the target protein in the cell's lysosome. In another embodiment, provided herein are methods of using the conjugates described herein to remove a polypeptide of interest (a target protein) from the extracellular space (the extracellular milieu) of a cell by sequestering the target protein in the cell's lysosome. In another embodiment, provided herein are methods of using the conjugates described herein to remove a polypeptide of interest (a target protein) from the surface of a cell by sequestering the target protein in the cell's lysosome and degrading the target protein. In another embodiment, provided herein are methods of using the conjugates described herein to remove a polypeptide of interest (a target protein) from the extracellular space (the extracellular milieu) of a cell by sequestering the target protein in the cell's lysosome and degrading the target protein.

Removal of a target protein may refer to reduction, or depletion, of the target protein from the cell surface or from the extracellular space, or the extracellular milieu, that is, a reduction, or depletion, of the amount of the target protein on the cell surface or in the extracellular milieu.

In one aspect, provided herein are methods of using the conjugates described herein to sequester a polypeptide of interest (a target protein) in a cell's lysosome. In one aspect, provided herein are methods of using the conjugates described herein to sequester a polypeptide of interest (a target protein) in a cell's lysosome and to degrade the polypeptide of interest.

In one aspect, provided herein are methods of using the conjugates described herein to degrade a polypeptide of interest (a target protein).

In one aspect, provided herein are methods of depleting a polypeptide of interest (a target protein) described herein by degradation through a cell's lysosomal pathway.

In another aspect, provided herein are methods of depleting a polypeptide of interest (a target protein) described herein by administering to a subject in need thereof an effective amount of a conjugate or pharmaceutically acceptable salt described herein, or a pharmaceutical composition described herein. In certain embodiments, the subject is a mammal (e.g., human).

In certain embodiments, the target protein is a VEGF protein, an EGFR protein, a VEGFR protein, a PD-L1 protein, an FGFR2 protein or an FGFR3 protein.

### 5.5.2. Uses of Bifunctional Molecules Including TNFα Binding Moieties

Also provided herein are methods of using the compounds described herein to remove TNFα from the extracellular milieu of a cellular sample or biological system. For example, the TNFα can be removed by sequestration in the cell's lysosome. In one embodiment, provided herein are methods of using the compounds described herein to remove TNFα from the extracellular space (the extracellular milieu) by sequestering the TNFα in the cell's lysosome and degrading the TNFα.

Removal of TNFα may refer to reduction, or depletion, of the TNFα from the cell surface or from the extracellular space, or the extracellular milieu, that is, a reduction, or depletion, of the amount of the TNFα on the cell surface or in the extracellular milieu.

In one embodiment, provided herein are methods of using the compounds described herein to degrade TNFα. In one aspect, provided herein are methods of depleting TNFα by degradation through a cell's lysosomal pathway.

The compounds of this disclosure including TNFα binding moieties can modulate the activity of TNFα. Accordingly, the compounds of this disclosure including TNFα binding moieties have utility in treating conditions associated with the modulation of TNFα. In one embodiment, provided herein are methods of treating a disease or disorder associated with TNFα by administering to a subject, e.g., a human, in need thereof an effective amount of a compound or pharmaceutically acceptable salt described herein, or a pharmaceutical composition described herein including TNFα binding moieties. The disease or disorder associated with TNFα for which the compounds could be of benefit include inflammatory and autoimmune disorders, neurological and neurodegenerative disorders, pain and nociceptive disorders, cardiovascular disorders, metabolic disorders, ocular disorders, and oncological disorders.

### 5.5.3. Methods of Treatment

More generally, provided herein are methods of treating a disease or disorder by administering to a subject, e.g., a human, in need thereof an effective amount of a bifunctional compound, conjugate or pharmaceutically acceptable salt described herein, or a pharmaceutical composition described herein.

The terms “administer”, “administration”, or “administering” refer to the act of injecting or otherwise physically delivering a substance (e.g., a conjugate or pharmaceutical composition provided herein) to a subject or a patient (e.g., human), such as by mucosal, topical, intradermal, parenteral, intravenous, intramuscular delivery and/or any other method of physical delivery described herein or known in the art. In a particular embodiment, administration is by intravenous infusion.

The terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result.

In certain embodiments, when the disorder or disease is cancer, “effective amount” or “therapeutically effective amount” mean that amount of a conjugate or pharmaceutical composition provided herein which, when administered to a human suffering from a cancer, is sufficient to effect treatment for the cancer. “Treating” or “treatment” of the cancer includes one or more of:

(1) limiting/inhibiting growth of the cancer, e.g. limiting its development;
(2) reducing/preventing spread of the cancer, e.g. reducing/preventing metastases;
(3) relieving the cancer, e.g. causing regression of the cancer,
(4) reducing/preventing recurrence of the cancer; and
(5) palliating symptoms of the cancer.

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

In certain embodiments, the disease or disorder is treated by depletion of the target protein by degradation through the lysosomal pathway.

In certain embodiments, the disease or disorder is treated by depletion of certain proteins, for example, soluble proteins, e.g., secreted proteins, cell surface proteins (for example, cell surface receptor proteins, e.g., tyrosine kinase receptors, soluble cytokine receptors, and immune checkpoint receptors, e.g., EGFR, VEGFR, FGFR, and PD-L1), lectins, complements, lipoproteins, transport proteins, MHC class I and class II molecules, cytokines, chemokines, and/or receptors, or fragments or subunits of any of the foregoing. In certain embodiments, the disease or disorder is treated by depletion of target TNFα in a human patient by degradation through the lysosomal pathway.

In certain embodiments, the disease or disorder is a cancer.

In certain embodiments, the cancer is selected from the group consisting of bladder cancer, breast cancer, cervical cancer, cholangiocarcinoma, endometrial cancer, hepatocellular carcinoma, kidney cancer, melanoma, myeloid neoplasms, non-small cell lung cancer (NSCLC), Ewing's sarcoma, and Hodgkin's Lymphoma.

In certain embodiments, the cancer is a solid tumor.

In certain embodiments, the disease or disorder is an inflammatory or autoimmune disease.

In certain embodiments, the disease or disorder is an inflammatory disease.

In certain embodiments, the disease or disorder is an autoimmune disease.

### 5.6. Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

The term “compounds” refers to at least one compound. For example, a compound of Formula (I) or (X) includes a compound of the formula, and/or two or more compounds of Formula (I).

Unless otherwise indicated, any heteroatom with unsatisfied valences is assumed to have hydrogen atoms sufficient to satisfy the valences.

Throughout the specification, groups and substituents thereof may be chosen by one skilled in the field to provide stable moieties and compounds.

The terms “halo” and “halogen,” refer to F, Cl, Br, and I.

The term “cyano” refers to the group —CN.

The term “amino” refers to the group —NH₂.

The term “hydroxy” refers to the group —OH.

The term “nitro” refers to the group —NO₂.

The term “oxo” refers to the group ═O.

The term “alkyl” refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₆ alkyl” denotes straight and branched chain alkyl groups with one to six carbon atoms.

The term “haloalkyl” is intended to include both branched and straight-chain saturated aliphatic hydrocarbon groups substituted with one or more halogen atoms. For example, “C₁₋₄ haloalkyl” is intended to include C₁, C₂, C₃, and C₄ alkyl groups substituted with one or more halogen atoms. Representative examples of haloalkyl groups include, but are not limited to, —CF₃, —CCl₃, —CFCl₂, and —CH₂CF₃.

The term “fluoroalkyl” is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “aminoalkyl” includes both branched and straight-chain saturated alkyl groups substituted with one or more amine groups. For example, “aminoalkyl” includes —CH₂NH₂, —CH₂CH₂NH₂, and C₁₋₄ aminoalkyl.

The term “alkenyl” refers to a straight or branched chain hydrocarbon radical containing from 2 to 12 carbon atoms and at least one carbon-carbon double bond.

Exemplary such groups include ethenyl or allyl. For example, “C₂₋₆ alkenyl” denotes straight and branched chain alkenyl groups with two to six carbon atoms.

The term “alkynyl” refers to a straight or branched chain hydrocarbon radical containing from 2 to 12 carbon atoms and at least one carbon to carbon triple bond.

Exemplary such groups include ethynyl. For example, “C₂₋₆ alkynyl” denotes straight and branched chain alkynyl groups with two to six carbon atoms.

The term “cycloalkyl,” as used herein, refers to a group derived from a saturated monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “cycloalkenyl,” as used herein, refers to a group derived from a non-aromatic monocyclic or polycyclic hydrocarbon molecule having at least one carbon-carbon double bond, by removal of one hydrogen atom from a saturated ring carbon atom. Representative examples of cycloalkenyl groups include, but are not limited to, cyclobutenyl, cyclopentenyl, and cyclohexenyl. When numbers appear in a subscript after the symbol “C”, the subscript defines with more specificity the number of carbon atoms that a particular cycloalkyl group may contain. For example, “C₄₋₆ cycloalkenyl” denotes cycloalkenyl groups with four to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “haloalkoxy” and “—O(haloalkyl)” represent a haloalkyl group as defined above attached through an oxygen linkage (—O—). For example, “C₁₋₄ haloalkoxy” is intended to include C₁, C₂, C₃, and C₄ haloalkoxy groups.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The terms “hydroxyalkoxy” and “—O(hydroxyalkyl)” represent a hydroxyalkyl group as defined above attached through an oxygen linkage (—O—). For example, “C₁₋₄ hydroxyalkoxy” is intended to include C₁, C₂, C₃, and C₄ hydroxyalkoxy groups.

The term “alkylthio,” refers to an alkyl group attached to the parent molecular moiety through a sulfur atom, for example, methylthio group (—SCH₃). For example, “C₁₋₃ alkylthio” denotes alkylthio groups with one to three carbon atoms.

The term “arylthio,” refers to an aryl group attached to the parent molecular moiety through a sulfur atom, for example, phenylthio group (—S(phenyl)).

The terms “carbocycle”, “carbocyclo”, “carbocyclic” or “carbocyclyl” are used interchangeably and refer to cyclic groups having at least one saturated or partially saturated non-aromatic ring wherein all atoms of all rings are carbon. The carbocyclyl ring may be unsubstituted or may contain one or more substituents as valence allows. Thus, the term includes nonaromatic rings such as for example, cycloalkyl, cycloalkenyl, and cycloalkynyl rings. Exemplary bicyclic carbocyclyl groups include, indanyl, indenyl, dihydronaphthalenyl, tetrahydronaphthenyl, hexahydronaphthalenyl, octahydronaphthalenyl, decahydronaphthalenyl, bicycloheptanyl, bicyclooctanyl, and bicyclononanyl.

The term “aryl” refers to a group of atoms derived from a molecule containing aromatic ring(s) by removing one hydrogen that is bonded to the aromatic ring(s). Heteroaryl groups that have two or more rings must include only aromatic rings. Representative examples of aryl groups include, but are not limited to, phenyl and naphthyl. The aryl ring may be unsubstituted or may contain one or more substituents as valence allows.

The term “benzyl” refers to a methyl group in which one of the hydrogen atoms is replaced by a phenyl group. The phenyl ring may be unsubstituted or may contain one or more substituents as valence allows.

The term “aryloxy” refers to an aryl group attached to the parent molecular moiety through an oxygen atom, for example, phenoxy group (—O(phenyl)).

The term “heteroatom” refers to oxygen (O), sulfur (S), and nitrogen (N).

The terms “heterocycle”, “heterocyclo”, “heterocyclic”, and “heterocyclyl” are used interchangeably and refer to cyclic groups having at least saturated or partially saturated non-aromatic ring and wherein one or more of the rings have at least one heteroatom (0, S or N), said heteroatom containing ring preferably having 1 to 3 heteroatoms independently selected from O, S, and/or N. The ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The heterocyclo group may be attached at any available nitrogen or carbon atom. The heterocyclo ring may be unsubstituted or may contain one or more substituents as valence allows.

Exemplary monocyclic heterocyclyl groups include pyrrolidinyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, dihydroisoindolyl, and tetrahydroquinolinyl.

The term “heteroaryl” refers to substituted and unsubstituted aromatic 5- or 6-membered monocyclic groups and 9- or 10-membered bicyclic groups that have at least one heteroatom (0, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

The term “spirocarbocyclo”, “spirocarbocyclic”, or “spirocarbocyclyl” refers to a carbocyclyl ring attached to the molecular moiety by a carbon atom in the carbocyclyl ring that is shared with the molecular moiety.

The term “spiroheterocyclo”, “spiroheterocyclic”, or “spiroheterocyclyl” refers to a heterocyclyl ring attached to the molecular moiety by a carbon atom in the heterocyclyl ring that is shared with the molecular moiety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) or (X) can be provided as amorphous solids or crystalline solids. Lyophilization can be employed to provide the compounds as amorphous solids.

It should further be understood that solvates (e.g., hydrates) of the compounds of Formula (I) or (X) are also within the scope of the present disclosure. The term “solvate” means a physical association of a compound of Formula (I) or (X) with one or more solvent molecules, whether organic or inorganic. This physical association includes hydrogen bonding. In certain instances, the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The terms “antibody” and “immunoglobulin” are terms of art and can be used interchangeably herein, and refer to a molecule with an antigen binding site that specifically binds an antigen.

In a certain embodiments, an isolated antibody (e.g., monoclonal antibody) described herein, or an antigen-binding fragment thereof, which specifically binds to a protein of interest, for example, EGFR, is conjugated to one or more lysosomal targeting moieties, for example, via a linker.

An “antigen” is a moiety or molecule that contains an epitope to which an antibody can specifically bind. As such, an antigen is also is specifically bound by an antibody. In a specific embodiment, the antigen, to which an antibody described herein binds, is a protein of interest, for example, EGFR (e.g., human EGFR), or a fragment thereof, or for example, an extracellular domain of EGFR (e.g., human EGFR).

An “epitope” is a term known in the art and refers to a localized region of an antigen to which an antibody can specifically bind. An epitope can be a linear epitope of contiguous amino acids or can comprise amino acids from two or more non-contiguous regions of the antigen.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

The term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (a), delta (6), epsilon (a), gamma (γ) and mu (p), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

The terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; Al-Lazikani et al., 1997, J Mol. Biol., 273: 927-948; Chothia et al., 1992, J Mol. Biol., 227: 799-817; Tramontano et al., 1990, J Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half-life modulation, conjugate function and complement binding. In another aspect, an antibody fragment is a monovalent antibody that has an in vivo half-life substantially similar to an intact antibody. For example, such an antibody fragment may comprise on antigen binding arm linked to an Fc sequence capable of conferring in vivo stability to the fragment.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ⁴C and/or ⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

### 5.7. Exemplary Embodiments

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

Notwithstanding the appended claims, aspects of the present disclosure are illustrated by the following clauses.

Clause 1. A TNFα-degrading compound of formula (I):

X-L-Y (I)

or a salt thereof,
wherein:
X is a moiety that binds a cell surface folate receptor;
L is a linker having a backbone of 10 to 50 atoms in length; and
Y is an allosteric desymmetrization TNFα inhibitor.

Clause 2. The compound of clause 1, wherein L has a backbone of 12 to 40 atoms in length.

Clause 3. The compound of clause 2, wherein L has a backbone of 16 to 32 atoms in length.

Clause 4. The compound of clause 3, wherein L has a backbone of 20 to 30 atoms in length.

Clause 5. The compound of any one of clauses 1 to 4, wherein Y is of formula (XII):

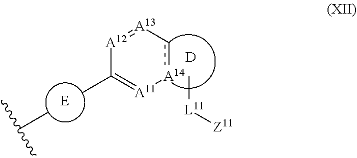

wherein:
A¹¹-A¹³ are independently selected from N and CR³⁰;
A¹⁴ is C or N;
D is a fused 5- or 6-membered ring selected from optionally substituted aryl, optionally substituted heteroaryl, and optionally substituted heterocycle;
E is selected from optionally substituted aryl, optionally substituted heteroaryl, optionally substituted cycloalkyl, and optionally substituted heterocycle;
L¹¹ is a linker comprising 1, 2 or 3 linking moieties each independently selected from optionally substituted (C₁-C₄)alkyl, —C(O)—, —O—, —S—, and —NR³⁶—;
Z¹¹ is a cyclic group selected from optionally substituted aryl, optionally substituted heteroaryl, optionally substituted cycloalkyl, and optionally substituted heterocycle;
each R³⁰ is independently selected from H, halogen (e.g., F), OH, optionally substituted (C₁-C₆)alkyl, optionally substituted (C₁-C₆)alkoxy, and CN; and
each R³⁶ is independently selected from H, and optionally substituted (C₁-C₆)alkyl.

Clause 6. The compound of any one of clauses 1 to 5, wherein Y is of formula (XIII):

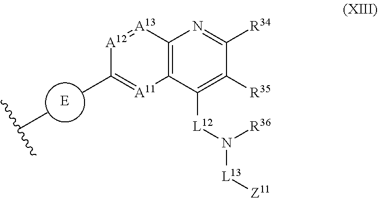

wherein:
A¹¹ is CR³¹ or N;
A¹² is CR³² or N;
A¹³ is CR³³ or N;
L¹² is a bond or —CH₂—;
L¹² is a bond or optionally substituted (C₁-C₃)alkyl;
E is a cyclic group selected from dihydropyridinonyl, phenyl, piperidinyl, pyrazinyl, pyrazolyl, pyridinyl, and pyrimidinyl, wherein the cyclic group E is optionally substituted;
Z¹¹ is a cyclic group selected from C₃₋₆ cycloalkyl, cyclopentenyl, phenyl, furanyl, imidazolyl, indolinyl, isoquinolinyl, isothiazolyl, isoxazolyl, oxazolyl, piperidinyl, pyrazinyl, pyrazolyl, pyridinyl, pyrimidinyl, thiazolyl, and thiophenyl, wherein the cyclic group Z¹¹ is optionally substituted; and
R³¹⁻³⁵ are independently selected from H, halogen, —CN, NO₂, optionally substituted C₁₋₆ alkyl, C₁₋₆ haloalkyl (e.g., —CF₃), and optionally substituted C₁₋₆ alkoxy (e.g., —OCF₃).

Clause 7. The compound of clause 6, wherein Y is of formula (XIV):

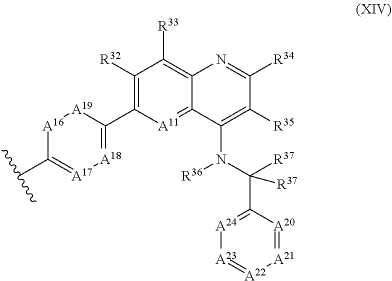

wherein:
A¹⁶ to A²⁴ are independently selected from CR⁵¹ and N;
each R³⁷ is independently selected from H, halogen (e.g., F), and optionally substituted (C₁-C₆)alkyl; and
each R⁵¹ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹, wherein each R²¹ is independently selected from H, and optionally substituted (C₁-C₆)alkyl.

Clause 8. The compound of clause 6, wherein Y is of formula (XV):

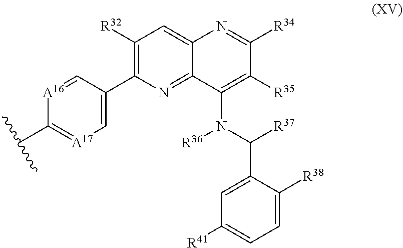

wherein:
A¹⁶ and A⁷ are independently selected from N and CH;
R³⁸ and R⁴¹ are independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
R³⁷ is selected from H, halogen, and optionally substituted (C₁-C₆)alkyl.

Clause 9. The compound of clause 7 or 8, wherein R³² is halogen.

Clause 10. The compound of any one of clauses 7 to 9, wherein R³⁴ and R³⁵ are independently selected from H, halogen and optionally substituted (C₁-C₆)alkyl.

Clause 11. The compound of clause 10, wherein:
R³⁴ is (C₁-C₃)alkyl (e.g., methyl); and
R³⁵ is halogen (e.g., chloride).

Clause 12. The compound of any one of clauses 7 to 11, wherein R³⁷ is not H, and the carbon atom to which R³⁷ is attached is of the (R) configuration.

Clause 13. The compound of any one of clauses 7 to 12, wherein R³⁷ is (C₁-C₃)alkyl.

Clause 14. The compound of any one of clauses 7 to 13, wherein R³⁶ is H.

Clause 15. The compound of any one of clauses 7 to 14, wherein R³⁸ is halogen.

Clause 16. The compound of any one of clauses 7 to 15, wherein R⁴¹ is CN.

Clause 17. The compound of any one of clauses 7 to 16, wherein Y is of formula (XVa):

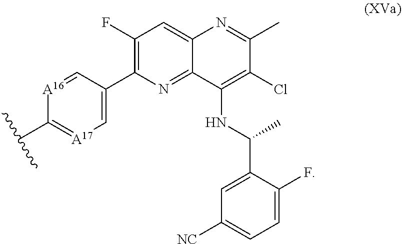

Clause 18. The compound of any one of clauses 7 to 17, wherein A¹⁶ and A⁷ are each N.

Clause 19. The compound of any one of clauses 7 to 17, wherein A¹⁶ is CH and A⁷ is N.

Clause 20. The compound of any one of clauses 7 to 17, wherein A¹⁶ and A¹⁷ are each CH.

Clause 21. The compound of any one of clauses 1 to 5, wherein Y is of formula (XVI):

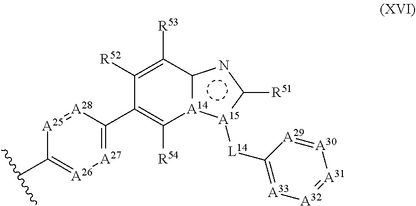

wherein:
A¹⁴ is C and A¹⁵ is N, or A¹⁴ is N and A⁵ is C;
A²⁵ to A³³ are independently selected from N and CR⁵⁵;
L¹⁴ is selected from —N(R⁵⁶)—and optionally substituted methylene (e.g., L¹⁴ is selected from —N(R⁵⁶)—, —CH₂—, —CH(OH)—, —CH(OCH₃)—, —CH(OCH₂CO₂H)—, —CH(NH₂)—, —CH(NHCOCH₃)—, —CH(CO₂H)—, —CH(CO₂benzyl)-, —CH(CH₃)—or —C(CH3)(OH)—);
R⁵¹ is selected from H, halogen, optionally substituted (C₁-C₆)alkyl, optionally substituted aryl, optionally substituted heteroaryl, optionally substituted cycloalkyl, and optionally substituted heterocycle;
R⁵²-R⁵⁵ are each independently selected from H, halogen (e.g., F), OH, optionally substituted (C₁-C₆)alkyl, optionally substituted (C₁-C₆)alkoxy, and CN; and
R⁵⁶ is selected from H, and optionally substituted (C₁-C₆)alkyl.

Clause 22. The compound of clause 21, wherein Y is of formula (XVII)—(XVIII):

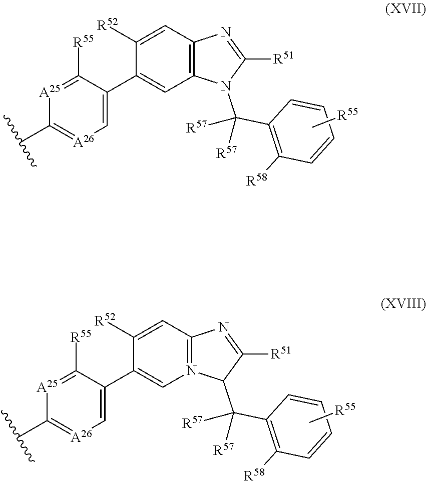

wherein:
each R⁵⁷ is independently selected from H, and optionally substituted (C₁-C₆)alkyl; and
R⁵⁸ is selected from H, halogen (e.g., F), OH, optionally substituted (C₁-C₆)alkyl (e.g., CF₃), optionally substituted (C₁-C₆)alkoxy, and CN.

Clause 23. The compound of clause 22, wherein Y is of formula (XVIIa)-(XVIIIa):

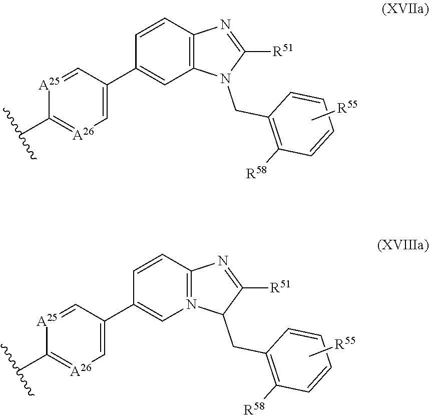

wherein R⁵⁸ is C₁₋₆ haloalkoxy.

Clause 24. The compound of any one of clauses 22 to 23, wherein A²⁵ and A²⁶ are each N.

Clause 25. The compound of any one of clauses 22 to 23, wherein A²⁵ is CH and A²⁶ is N.

Clause 26. The compound of any one of clauses 22 to 23, wherein A²⁵ and A²⁶ are each CH.

Clause 27. The compound of any one of clauses 22 to 26, wherein R⁵⁸ is —OCHF₂.

Clause 28. The compound of any one of clauses 22 to 27, wherein R⁵¹ is —C₁₋₆ alkyl-O-(optionally substituted aryl).

Clause 29. The compound of any one of clauses 22 to 27, wherein R⁵¹ is —C₁₋₆ alkyl.

Clause 30. The compound of clause 28 or 29, wherein Y has one of the following structures:

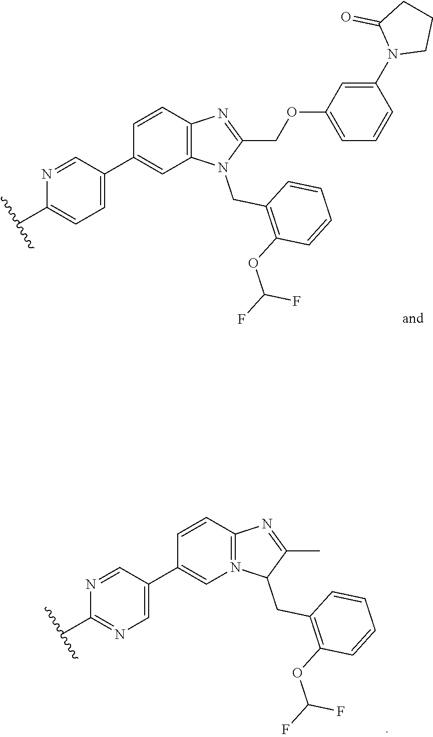

Clause 31. The compound of any one of clauses 1 to 24, wherein L comprises one or more linking moieties independently selected from —C₁₋₆-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, —NH—, and —NMe—, wherein p is 1 to 10.

Clause 32. The compound of clause 31, wherein L comprises repeating ethylene glycol moieties (e.g., —CH₂CH₂O—or —OCH₂CH₂—).

Clause 33. The compound of clause 32, wherein L comprises 1 to 20 ethylene glycol moieties.

Clause 34. The compound of clause 33, wherein L comprises 2 to 10 ethylene glycol moieties.

Clause 35. The compound of clause 34, wherein L comprises 6 to 10 ethylene glycol moieties.

Clause 36. The compound of clause 35, wherein L comprises 4 to 6 ethylene glycol moieties.

Clause 37. The compound of any one of clauses 1 to 36, wherein L is of formula (XIX):

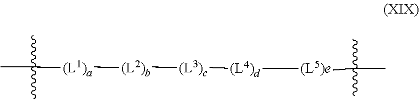

wherein
each L¹ to L⁵ is independently a linking moiety which together provide a linear linker between X and Y;
a is 1 or 2;
b, c, d, and e are each independently 0, 1, or 2.

Clause 38. The compound of clause 37, wherein -(L¹)ₐ- comprises an optionally substituted alkyl or ethylene glycol linking moiety.

Clause 39. The compound of clause 37 or 38, wherein each L′ is independently selected from:

—C₁₋₆-alkylene-, —(CH₂CH₂O)ₜ—, —C₁₋₆-alkylene-NHCO—, —C₁₋₆-alkyleneCONH—, and OCH₂, wherein t is 1 to 20.

Clause 40. The compound of any one of clauses 37 to 39, wherein:
each L² is independently selected from —NR²¹CO—C₁₋₆-alkylene-, —CONR²¹—C₁₋₆-alkylene,

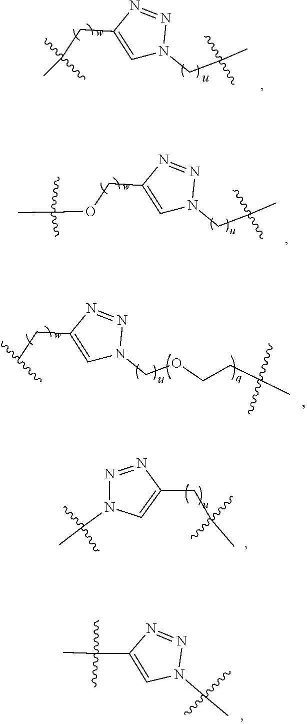

—OCH₂—, and —(OCH₂CH₂)_{q}—, wherein q is 1 to 10, u is 0 to 10, w is 1 to 10, and R²¹ is independently selected from H, and optionally substituted (C₁-C₆)alkyl; and
each L³ and L⁴ is independently absent or selected from —C₁₋₆-alkylene-, —(CH₂CH₂O)ₜ—, —C₁₋₆-alkylene-NHCO—, —C₁₋₆-alkyleneCONH—, and OCH₂, wherein t is 1 to 20.

Clause 41. The compound of any one of clauses 37 to 40, wherein:
each L⁵ is independently selected from —CH₂O—; —(CH₂CH₂O)ₜ—, —NR²¹CO—, —C₁₋₆-alkylene-,

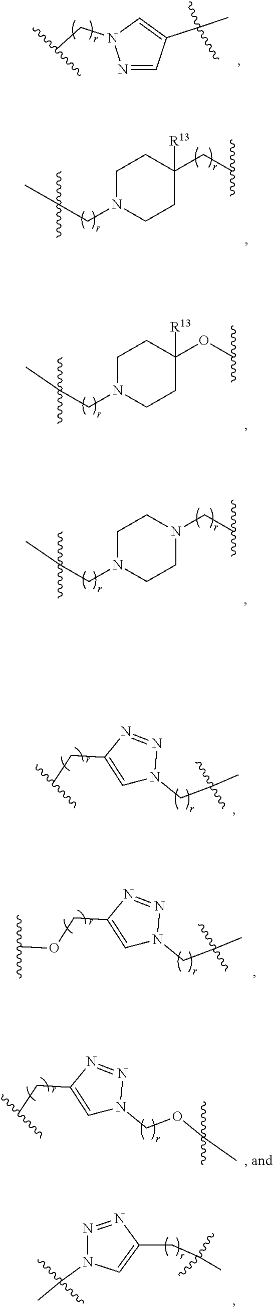

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
each r independently 0 to 20;
R²¹ is selected from H, and optionally substituted (C₁-C₆)alkyl.

Clause 42. The compound of any one of clauses 37 to 41, wherein a is 1.

Clause 43. The compound of any one of clauses 37 to 42, wherein at least one of b, c, d, and e is not 0.

Clause 44. The compound of any one of clauses 37 to 43, wherein b, d, and e are each independently 1 or 2.

Clause 45. The compound of any one of clauses 37 to 44, wherein a, b, d, and e are each 1, and c is 0.

Clause 46. The compound of any one of clauses 1 to 45, wherein L is a non-cleavable linker.

Clause 47. The compound of any one of clauses 1 to 46, wherein the linker L is selected from any one of the structures of Table 3.

Clause 48. The compound of any one of clauses 1 to 47, wherein X is of formula (Ia):

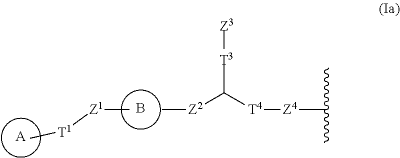

wherein:
A is a ring system of formula (II):

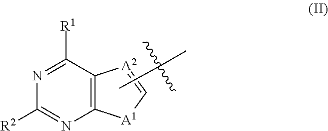

or a tautomer thereof, wherein:
R¹ and R² are independently selected from OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl
T¹ is an optionally substituted (C₁-C₃)alkylene;
Z¹ is selected from —NR²³—, —O—, —S—, and optionally substituted (C₁-C₃)alkylene, wherein R²³ is H, or optionally substituted (C₁-C₆)alkyl, or R²³ is cyclically linked with a ring atom of the B-ring to provide a fused 5 or 6 membered heterocycle ring optionally substituted;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted urea, optionally substituted thiourea, optionally substituted sulfonamide, —NR²—, —O—, —S—, and optionally substituted (C₁-C₆)alkylene;
Z³ is carboxyl or carboxyl bioisostere, or a prodrug thereof;
T³ is absent, or is selected from optionally substituted (C₁-C₆)alkylene;
T⁴ is optionally substituted (C₁-C₆)alkylene, or is absent;
Z⁴ is a linking moiety;
each R²¹ is independently selected from H, and optionally substituted (C₁-C₆)alkyl.

Clause 49. A TNFα-degrading compound of formula (Ia):

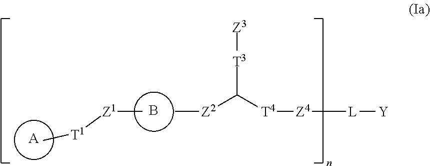

or a salt thereof,
wherein:
Y is a small molecule TNFα inhibitor;
L is a linker;
n is 1 to 3;
A is a ring system of formula (II):

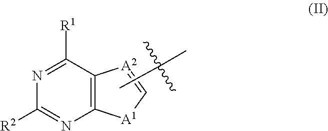

or a tautomer thereof, wherein:
   R¹ and R² are independently selected from H, OH, NR²¹, and optionally substituted (C₁-C₆)alkyl (e.g., —CH₃ or —CH₂OH);
   A¹ is selected from —N═CR³—, —CR³═N—, —CR³═CR³—, NR²¹, S, O, and C(R⁴)₂;
   A² is selected from N, and CR³;
   each R³ is independently selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
   each R⁴ is independently selected from H, halogen, and optionally substituted (C₁-C₆)alkyl;
T¹ is an optionally substituted (C₁-C₃)alkylene;
Z¹ is selected from —NR²³—, —O—, —S—, and optionally substituted (C₁-C₃)alkylene, wherein R²³ is H, or optionally substituted (C₁-C₆)alkyl, or R²³ is cyclically linked with a ring atom of the B-ring to provide a fused 5 or 6 membered heterocycle ring optionally substituted;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted urea, optionally substituted thiourea, optionally substituted sulfonamide, —NR²¹—, —O—, —S—, and optionally substituted (C₁-C₆)alkylene;
Z³ is carboxyl or carboxyl bioisostere, or a prodrug thereof;
T³ is absent, or is selected from optionally substituted (C₁-C₆)alkylene;
T⁴ is optionally substituted (C₁-C₆)alkylene, or is absent;
Z⁴ is a linking moiety; and
each R²¹ is independently selected from H, and optionally substituted (C₁-C₆)alkyl; wherein at least one of following applies:
1) T³ is optionally substituted (C₁-C₆)alkylene;
2) L is a non-cleavable linker and Y is an allosteric desymmetrization TNFα inhibitor;
3) when A is of formula (II-A) or (II-A′), or a tautomer thereof:

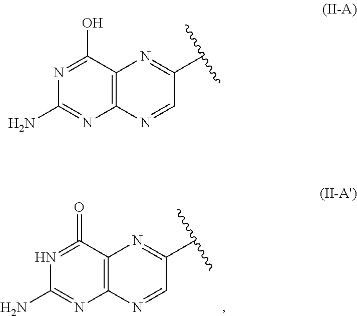

then Z¹ is not NR²¹, and/or B is not 1,4-linked phenyl;
4) when A is of formula (XII—B), or a tautomer thereof:

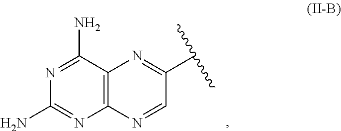 [SMILES: *c1cnc2nc(N)nc(N)c2n1]

then Z¹ is not NR²¹, and/or B is not phenyl; and/or
5) when A is of formula (II-C) or (II-C′), or a tautomer thereof:

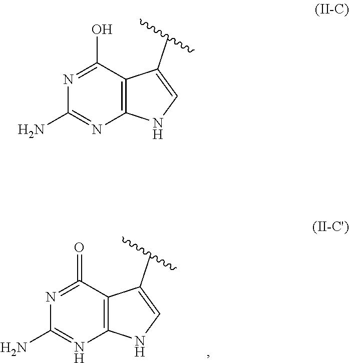

then T¹-Z¹ is not —CH₂CH₂—, and/or B is not 1,4-linked phenyl.

Clause 50. The compound of clause 49, wherein L has a backbone of 10 to 100 atoms in length (e.g., 1 to 50, 12 to 40, 16 to 32, or 20 to 30 atoms in length).

Clause 51. The compound of clause 49 or 50, wherein n is 1.

Clause 52. The compound of any one of clauses 48 to 51, wherein T³ is optionally substituted (C₁-C₆)alkylene.

Clause 53. The compound of clause 52, wherein T³ is (C₁-C₃)alkylene.

Clause 54. The compound of clause 53, wherein T³ is —CH₂CH₂—.

Clause 55. The compound of any one of clauses 48 to 54, wherein T⁴ is absent.

Clause 56. The compound of any one of clauses 48 to 55, wherein T³ is absent.

Clause 57. The compound of clause 56, wherein T⁴ is optionally substituted (C₁-C₆)alkylene.

Clause 58. The compound of clause 57, wherein T⁴ is (C₁-C₃)alkylene.

Clause 59. The compound of clause 57, wherein T⁴ is —CH₂CH₂—or —CH₂—.

Clause 60. The compound of any one of clauses 48 to 59, wherein Z³ is selected from —COOH, —COOR²², —CH₂OH, —CH₂OR²², —CN, and tetrazole, wherein R²¹ is optionally substituted (C₁-C₆)alkyl.

Clause 61. The compound of clause 60, wherein Z³ is selected from:

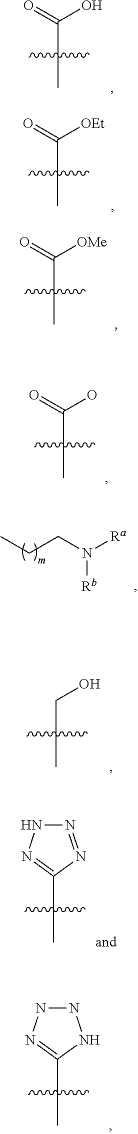

wherein:
R^{a} and R^{b} are independently selected from H and optionally substituted (C₁-C₆)alkyl, or R^{a} and R^{b} are cyclically linked to provide an optionally substituted 5 or 6-membered heterocycle; and
m is 1 to 5.

Clause 62. The compound of clause 61, wherein Z³ is COOH.

Clause 63. The compound of clause 61, wherein Z³ is

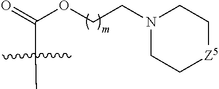

wherein
Z⁵ is CH₂, O, NH or NR²¹; and
R²¹ is (C₁-C₆)alkyl.

Clause 64. The compound of clause 63, wherein Z⁵ is O, NH or NMe, and m is 1.

Clause 65. The compound of any one of clauses 48 to 64, wherein Z² is —CONR²¹—, —NR²¹CO—, —SO₂NR²¹—, —NR²¹C(═O)NR²¹—, or —NR²¹C(═S)NR²¹, wherein each R²¹ is independently selected from H, and optionally substituted (C₁-C₆)alkyl.

Clause 66. The compound of any one of clauses 48 to 65, wherein Z⁴ is a linking moiety selected from one or more of —CONR²¹—, —SO₂NR²¹—, —NR²¹—, —O—, —S—, optionally substituted aryl (e.g., 1,4-phenyl) and optionally substituted heteroaryl (e.g., oxadiazole or triazole), wherein R²¹ is selected from H, and optionally substituted (C₁-C₆)alkyl.

Clause 67. The compound of clause 66, wherein Z⁴ is a linking group selected from:

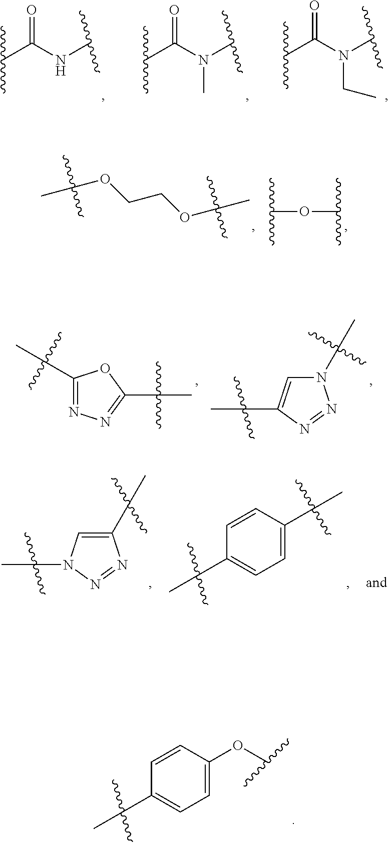

Clause 68. The compound of any one of clauses 48 to 55, wherein —Z²CH(-T³-Z³)T⁴Z⁴— of formula (X) or (XI) is selected from the following structures:

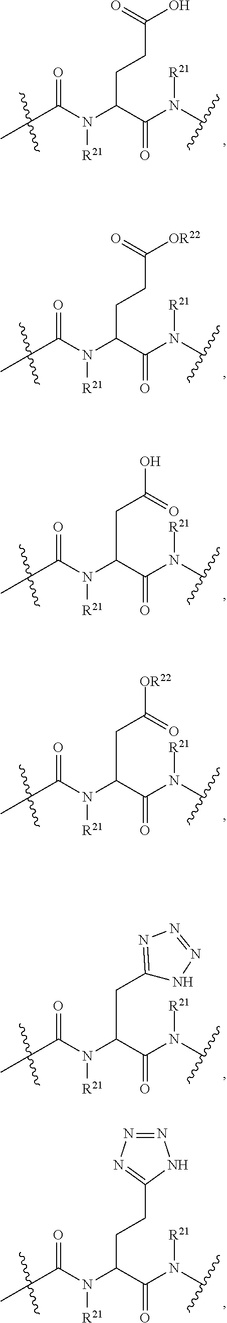

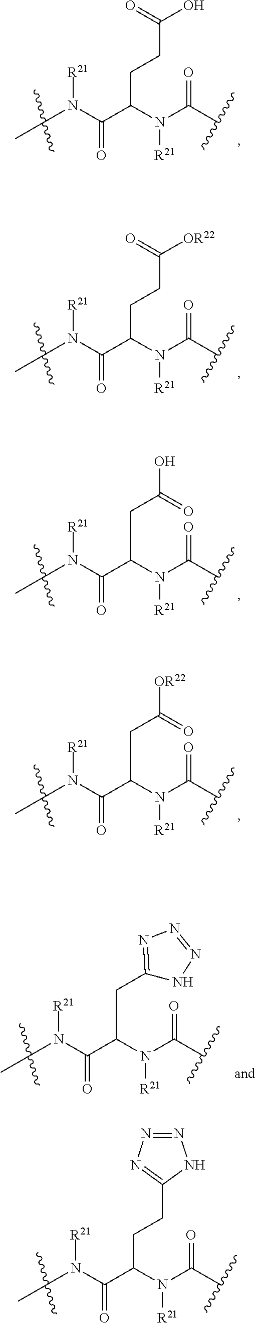

or a tautomer thereof, or a salt thereof.

Clause 69. The compound of clause 59, wherein —Z²CH(-T³-Z³)T⁴Z⁴—of formula (I) or (Ia) is selected from the following structures:

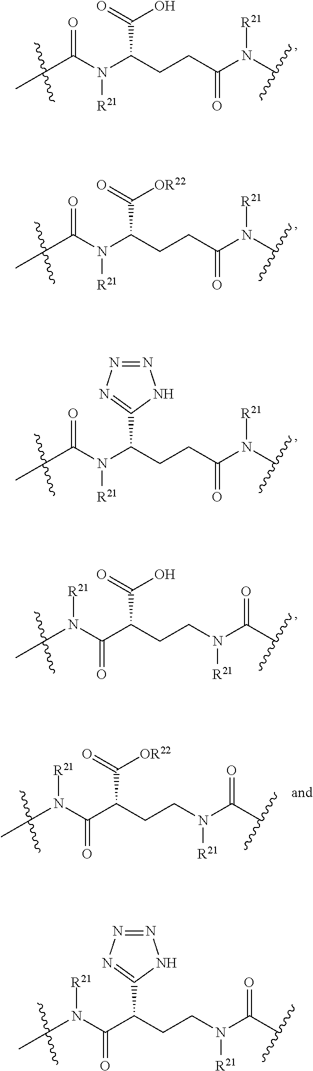

or a tautomer thereof, or a salt thereof.

Clause 70. The compound of any one of clauses 48 to 69, wherein A¹ of ring system A of formula (XII) is —N═CR³—, —CR³═N—, or —CR³═CR³—.

Clause 71. The compound of clause 70, wherein A of formula (II) is of formula (III):

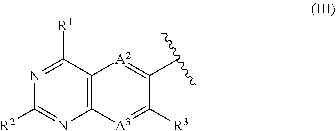

or a tautomer thereof, or a salt thereof, wherein:
A² is selected from N, and CR³;
A³ is selected from N, and CR¹.

Clause 72. The compound of clause 71, wherein A² and A³ are each N.

Clause 73. The compound of clause 71, wherein A² and A³ are each independently CR³.

Clause 74. The compound of any one of clauses 71 to 73, wherein each R³ is H.

Clause 75. The compound of any one of clauses 71 to 74, wherein R² is —NH₂.

Clause 76. The compound of any one of clauses 71 to 74, wherein R² is optionally substituted (C₁-C₆)alkyl.

Clause 77. The compound of any one of clauses 71 to 76, wherein R² is —CH₃ or —CH₂OH.

Clause 78. The compound of any one of clauses 71 to 77, wherein R¹ is OH.

Clause 79. The compound of any one of clauses 71 to 78, wherein A is selected from:

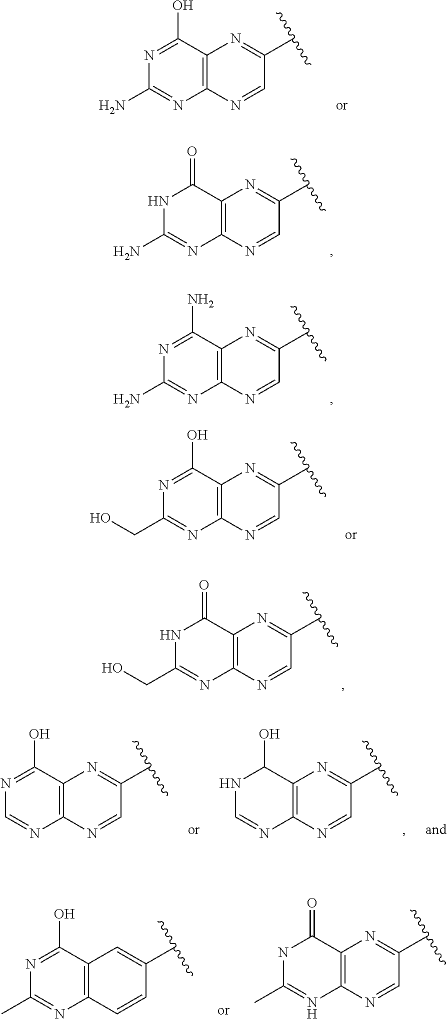

or a tautomer thereof.

Clause 80. The compound of any one of clauses 48 to 69, wherein A¹ of ring system A of formula (II) is NR²¹, S, O, or C(R²)₂.

Clause 81. The compound of clause 80, wherein A is of formula (IVa) or (IVb):

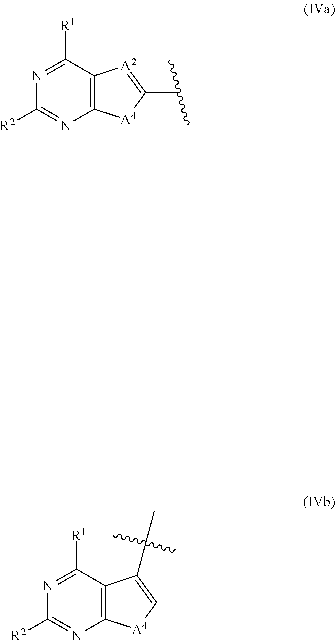

or a tautomer thereof, or a salt thereof, wherein A⁴ is selected from NR²¹, S, and O.

Clause 82. The compound of clause 81, wherein A⁴ is NR²¹.

Clause 83. The compound of clause 81 or 82, wherein A² is CR³.

Clause 84. The compound of any one of clauses 80 to 83, wherein R² is —NH₂.

Clause 85. The compound of any one of clauses 80 to 83, wherein R² is optionally substituted (C₁-C₆)alkyl (e.g., —CH₃ or —CH₂OH).

Clause 86. The compound of any one of clauses 80 to 85, wherein R¹ is OH.

Clause 87. The compound of any one of clauses 80 to 86, wherein A is selected from:

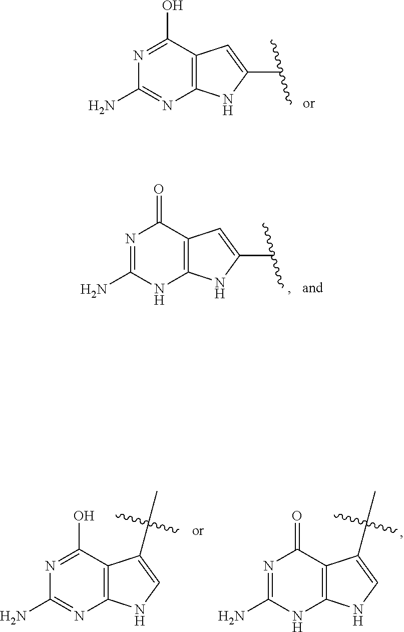

or a tautomer thereof.

Clause 88. The compound of any one of clauses 48 to 87, wherein T¹ of formula (I) or (Ia) is CH₂.

Clause 89. The compound of any one of clauses 48 to 88, wherein Z¹ of formula (I) or (Ia) is NR²¹.

Clause 90. The compound of clause 89, wherein R²¹ is H.

Clause 91. The compound of clause 89, wherein R²¹ is methyl, ethyl, propyl, or propargyl.

Clause 92. The compound of any one of clauses 48 to 88, wherein Z¹ of formula (I) or (Ia) is O or S.

Clause 93. The compound of any one of clauses 48 to 88, wherein T¹-Z¹ of formula (I) or (Ia) is optionally substituted (C₁-C₆)alkylene.

Clause 94. The compound of clause 93, wherein T¹-Z¹ is —CH₂CH₂—.

Clause 95. The compound of clause 93, wherein T¹-Z¹ is —CH₂CH₂CH₂CH₂—or —CH₂CH₂CH₂—.

Clause 96. The compound of any one of clauses 48 to 95, wherein B of formula (I) or (Ia) is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, and optionally substituted bicycloalkyl (e.g., bicyclo[1.1.1]pentane).

Clause 97. The compound of clause 96, wherein B is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

Clause 98. The compound of clause 96 or 97, wherein —B—Z²—of formula (I) or (Ia) is selected from:

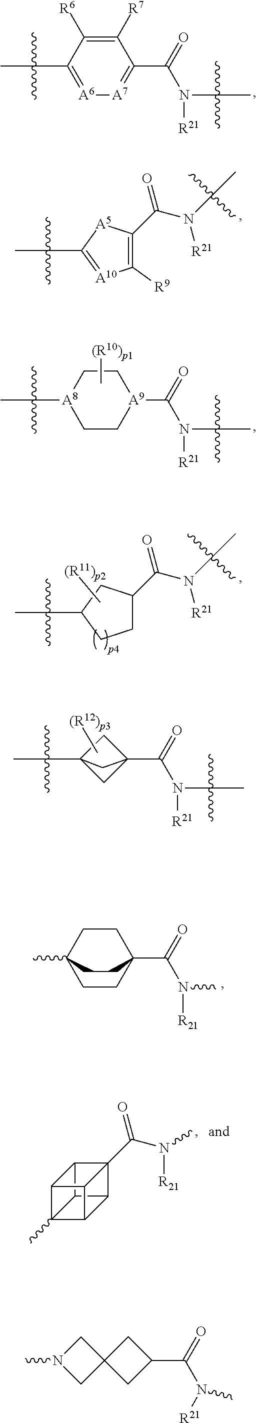

wherein:
A⁵ is selected from NR²¹, S, O, C(R²²)₂;
A⁶-A⁹ are independently selected from N, and CR²²;
A¹⁰ is selected from N, and CR;
R²¹ is selected from H, and optionally substituted (C₁-C₆)alkyl;
each R⁶ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

Clause 99. The compound of clause 98, wherein B—Z² is:

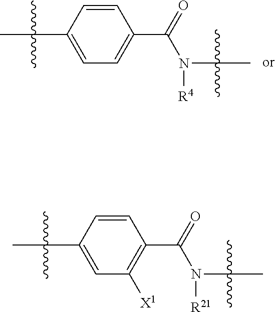

wherein X¹ is halogen.

Clause 100. The compound of clause 48, wherein A-T¹-Z¹—B—is selected from one of the following:

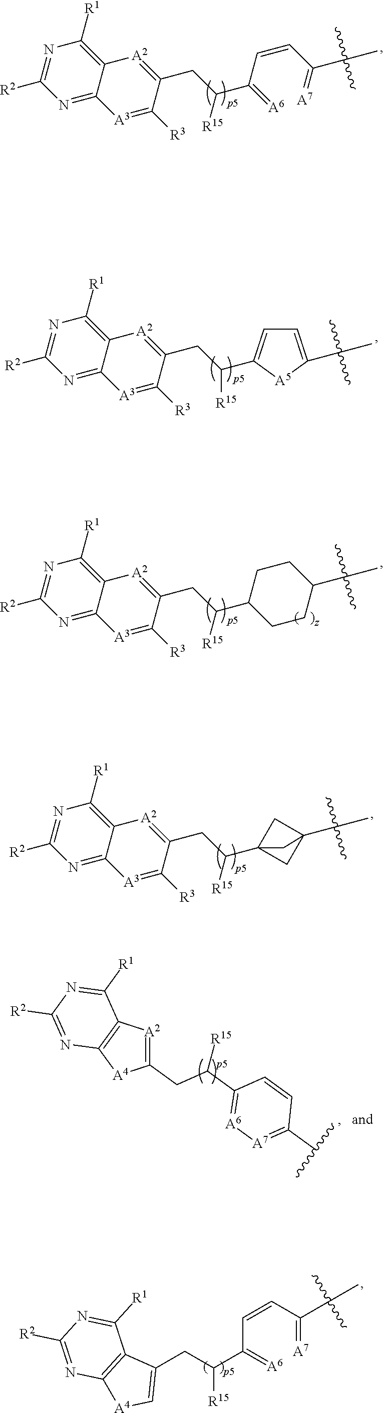

wherein:
A⁵ is selected from NR²¹, S, O, C(R⁵)₂;
A⁶ and A⁷ are independently selected from N, and, CR⁵;
z is 0 to 3
each R⁵ and R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

Clause 101. The compound of any one of clauses 1 to 49, wherein X of formula (I) or formula (Ia) comprises one of the following structures:

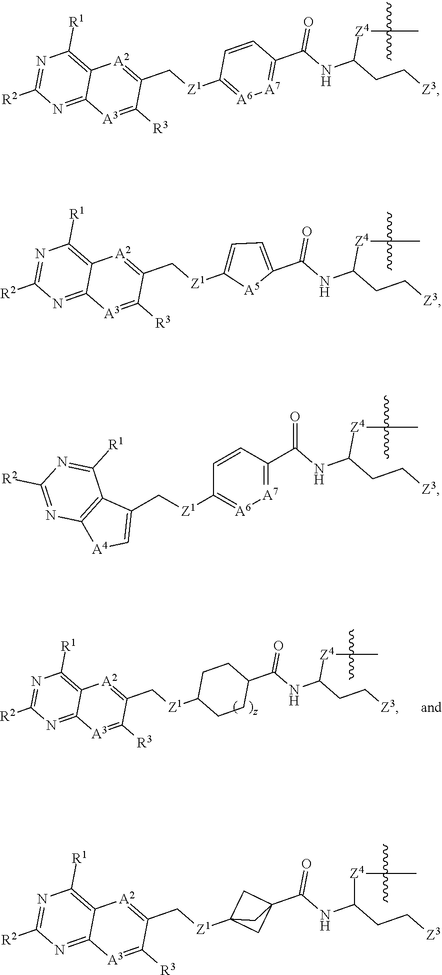

wherein:
A⁵ is selected from NR, S, O, C(R²²)₂;
A⁶ and A⁷ are independently selected from N, and, CR²²; and
z is 0 to 3.

Clause 102. The compound of any one of clauses 1 to 49, wherein X of formula (I) or formula (Ia) comprises one of the following structures:

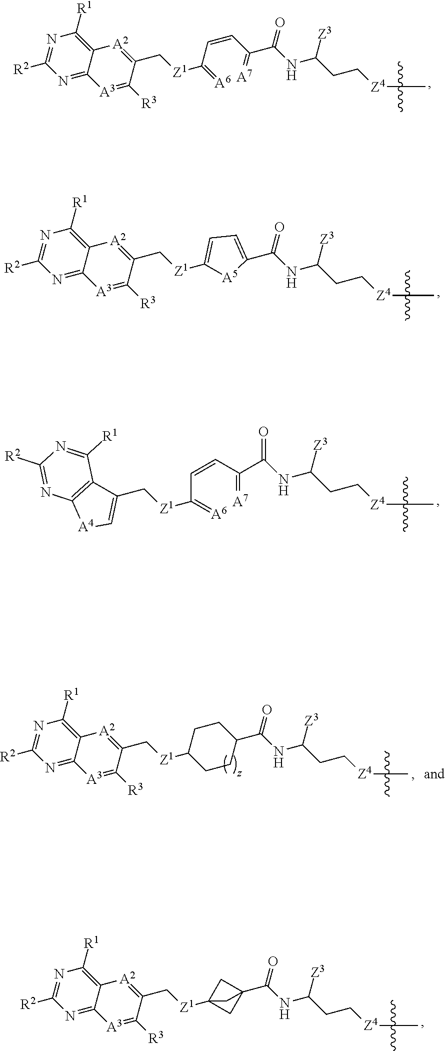

wherein:
A⁵ is selected from NR²¹, S, O, C(R²²)₂;
A⁶ and A⁷ are each independently selected from N, and, CR²; and
z is 0 to 3.

Clause 103. The compound of any one of clauses 1 to 101, wherein X of formula (I) or formula (Ia) comprises a structure of Table 1 or 2, or a tautomer thereof, or a salt thereof.

Clause 104. The compound of any one of clauses 1 to 101, wherein Y is as defined in any one of claims 5 to 30.

Clause 105. The compound on any one of clauses 1 to 103, wherein the compound of formula (I) is of one of Tables 4 to 6, or a tautomer thereof, or a salt thereof.

Clause 106. A method of internalizing TNFα protein in a cell, the method comprising:
contacting a sample that comprises cells having a cell surface folate receptor and extracellular TNFα protein with an effective amount of a compound according to any one of clauses 1 to 104, wherein the compound specifically binds the TNFα protein and specifically binds the cell surface folate receptor to facilitate cellular uptake of the TNFα protein.

Clause 107. A method of reducing levels of TNFα protein in a biological system, the method comprising contacting the biological system with an effective amount of a compound according to any one of clauses 1 to 104, wherein the compound specifically binds the TNFα protein and specifically binds a cell surface receptor of cells in the biological system to facilitate cellular uptake and degradation of the TNFα protein.

Clause 108. The method of clause 107, wherein the biological system comprises cells that comprise a folate cell surface receptor.

Clause 109. The method of clause 107 or 108, wherein the biological system is a human subject.

Clause 110. The method of any one of claims 107 to 109, wherein the biological system is an in vitro cellular sample.

Clause 111. A method of treating a disease or disorder associated with a TNFα protein, the method comprising administering to a subject in need thereof an effective amount of a compound according to any one of clauses 1 to 104, wherein the compound specifically binds the TNFα protein.

Clause 112. The method of clause 111, wherein the disease or disorder is an inflammatory disease.

Clause 113. The method of clause 111, wherein the disease or disorder is an autoimmune disease.

Clause 114. The method of clause 111, wherein the disease or disorder is cance

Clause 201. A cell surface folate receptor binding compound of formula (I):

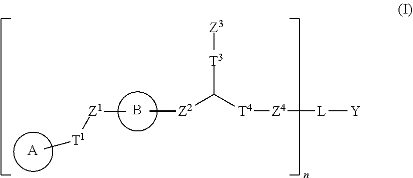

or a salt thereof, wherein:
T¹ is an optionally substituted (C₁-C₃)alkylene;
   Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
   B is a ring system selected from optionally substituted aryl, optionally substituted heteroaryl, optionally substituted heterocycle, optionally substituted cycloalkyl, and optionally substituted bridged bicycle;
   Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is absent, carboxyl or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety (e.g., a linking moiety selected from ester, amide, sulfonamide, urea, thiourea, amine, ether, optionally substituted aryl, optionally substituted heterocycle, and optionally substituted heteroaryl);
each R²¹ is independently selected from H, and optionally substituted (C₁-C₆)alkyl;
   n is 1 to 100;
L is a linker;
Y is a moiety of interest; and
A is a ring system of formula (II):

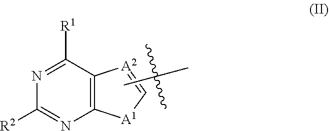

or a tautomer thereof, wherein:
R¹ and R² are independently selected from H, OH, NR²¹, and optionally substituted (C₁-C₆)alkyl (e.g., —CH₃ or —CH₂OH);
A¹ is selected from —N═CR³—, —CR³═N—, —CR³═CR³—, NR²¹, S, O, and C(R⁴)₂;
A² is selected from N, and CR³;
each R³ is independently selected from H, halogen (e.g., F), OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen (e.g., F), and optionally substituted (C₁-C₆)alkyl;
with the proviso that at least one of following applies:
1) T³ is optionally substituted (C₁-C₆)alkylene (e.g., —CH₂CH₂—);
2) L is a non-cleavable linker and Y is an extracellular target-binding moiety;
3) when A is of formula (II-A) or (II-A′), or a tautomer thereof:

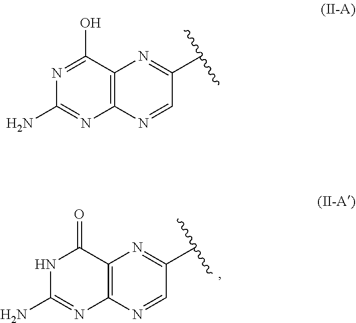 [SMILES: *c1cnc2nc(N)nc(O)c2n1 ; *c1cnc2nc(N)[nH]c(=O)c2n1]

then Z is not NR²¹, and/or B is not 1,4-linked phenyl;
4) when A is of formula (II-B), or a tautomer thereof:

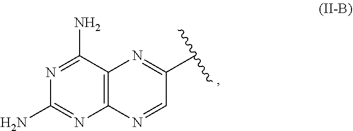 [SMILES: *c1cnc2nc(N)nc(N)c2n1]

then Z¹ is not NR²¹, and/or B is not 1,4-linked phenyl; and/or
5) when A is of formula (II-C) or (II-C′), or a tautomer thereof:

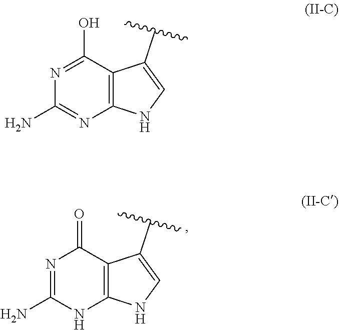 [SMILES: *c1c[nH]c2nc(N)nc(O)c12 ; *c1c[nH]c2[nH]c(N)nc(=O)c12 ; ","]

then T¹-Z¹ is not —CH₂CH₂—, and/or B is not phenyl.

Clause 202. The compound of clause 201, wherein T³ is optionally substituted (C₁-C₆)alkylene.

Clause 203. The compound of clause 202, wherein T³ is (C₁-C₃)alkylene.

Clause 204. The compound of clause 203, wherein T³ is —CH₂CH₂—.

Clause 205. The compound of any one of clauses 201 to 204, wherein T⁴ is absent.

Clause 206. The compound of clause 205, wherein the compound is of formula (IIIA):

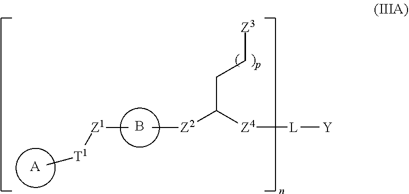

wherein p is 0 or 1.

Clause 207. The compound of clause 201, wherein T³ is absent.

Clause 208. The compound of clause 207, wherein T⁴ is optionally substituted (C₁-C₆)alkylene.

Clause 209. The compound of clause 208, wherein T⁴ is (C₁-C₃)alkylene.

Clause 210. The compound of clause 209, wherein T⁴ is —CH₂CH₂—.

Clause 211. The compound of any one of clauses 207 to 210, wherein the compound is of formula (IIIB):

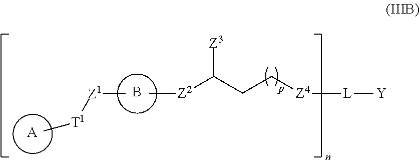

wherein p is 0 or 1.

Clause 212. The compound of any one of clauses 201 to 211, wherein Z³ is selected from —COOH, —COOR²², —CH₂OH, —CH₂OR²², —CN, and tetrazole, wherein R²² is optionally substituted (C₁-C₆)alkyl.

Clause 213. The compound of clause 212, wherein Z³ is selected from:

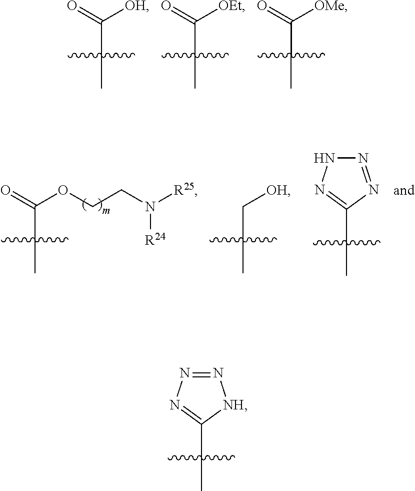

wherein:
R²⁴ and R²⁵ are independently selected from H and optionally substituted (C₁-C₆)alkyl, or R²⁴ and R²⁵ are cyclically linked to provide an optionally substituted 5 or 6-membered heterocycle; and
m is 1 to 5.

Clause 214. The compound of clause 213, wherein Z³ is COOH.

Clause 215. The compound of clause 213, wherein Z³ is

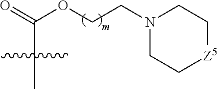

wherein
Z⁵ is O, NH or NR²¹; and
R²¹ is (C₁-C₆)alkyl.

Clause 216. The compound of clause 215, wherein Z⁵ is O, NH or NMe, and m is 1.

Clause 217. The compound of any one of claims 201 to 210, wherein Z³ is absent, and the compound is of formula (IIIC):

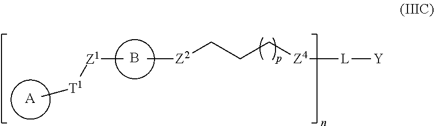

wherein p is 0 or 1.

Clause 218. The compound of clause 210, wherein Z² is —CONR²¹—, wherein R²¹ is selected from H, and optionally substituted (C₁-C₆)alkyl.

Clause 219. The compound of any one of clauses 212 to 118, wherein Z² is —CONR²¹—, —NR²¹CO—, —SO₂NR²¹—, —NR²¹C(═O)NR²¹—, or —NR²¹C(═S)NR²¹, wherein each R²¹ is independently selected from H, and optionally substituted (C₁-C₆)alkyl.

Clause 220. The compound of any one of clauses 212 to 219, wherein Z⁴ is a linking moiety selected from —CONR²¹—, —NR²¹—, —O—, —S—, optionally substituted aryl (e.g., 1,4-phenyl) and optionally substituted heteroaryl (e.g., oxadiazole or triazole), wherein R²¹ is selected from H, and optionally substituted (C₁-C₆)alkyl.

Clause 221. The compound of clause 220, wherein Z⁴ is a linking group selected from:

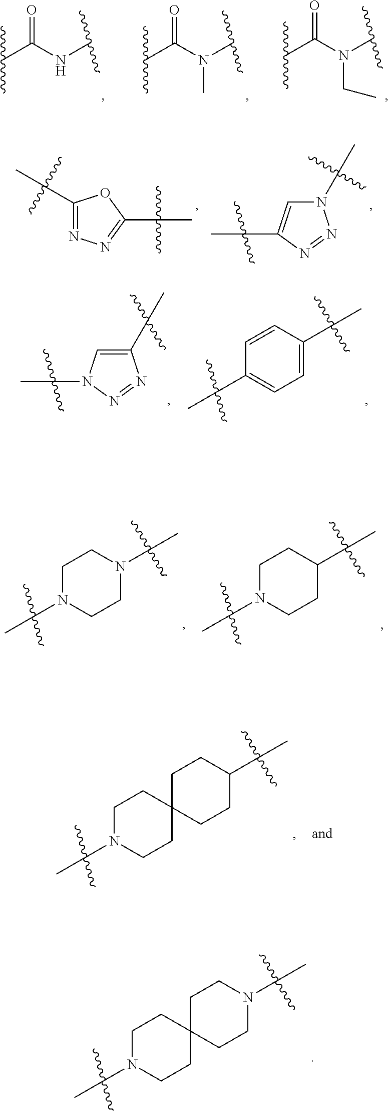

Clause 222. The compound of any one of clauses 112 to 121, wherein —Z²CH(-T³-Z³)T⁴Z⁴—of formula (I) is selected from the following structures:

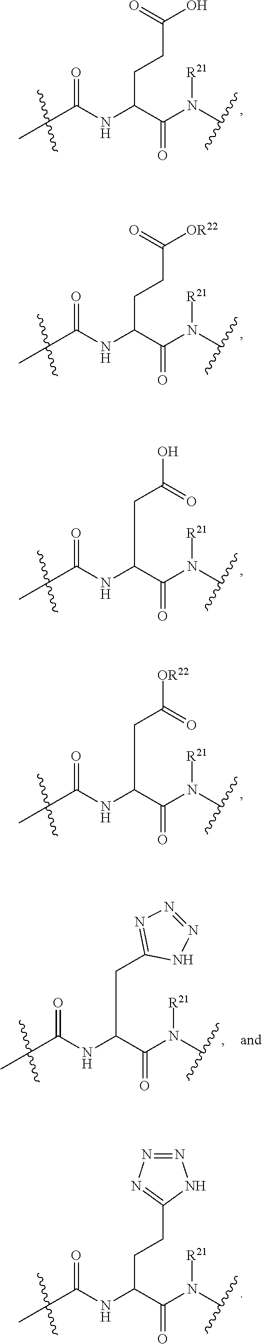

or a tautomer thereof, or a salt thereof.

Clause 223. The compound of any one of clauses 112 to 121, wherein —Z²CH(-T³-Z³)T⁴Z⁴—of formula (I) is selected from the following structures:

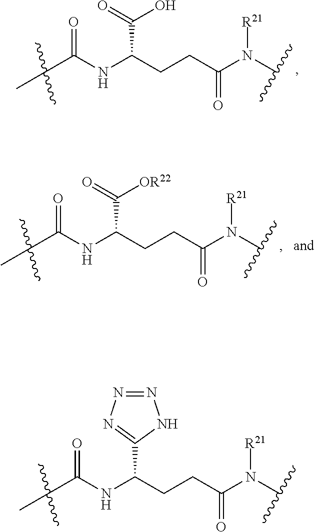

or a tautomer thereof, or a salt thereof.

Clause 224. The compound of any one of clauses 112 to 121, wherein —Z²CH(-T³-Z³)T⁴Z⁴—of formula (I) is selected from the following structures:

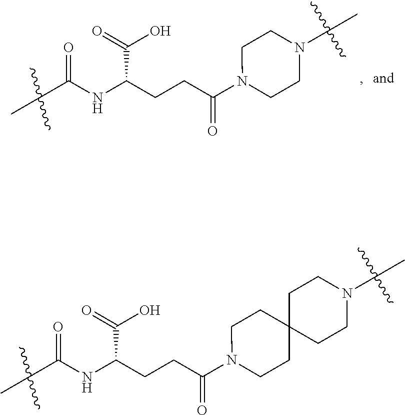 [SMILES: *C(=O)N[C@@H](CCC(=O)N1CCN(*)CC1)C(=O)O ; *C(=O)N[C@@H](CCC(=O)N1CCC2(CCN(*)CC2)CC1)C(=O)O]

or a tautomer thereof, or a salt thereof.

Clause 226. The compound of any one of clauses 212 to 221, wherein —Z²CH(-T³-Z³)T⁴Z⁴—of formula (I) is selected from the following structures:

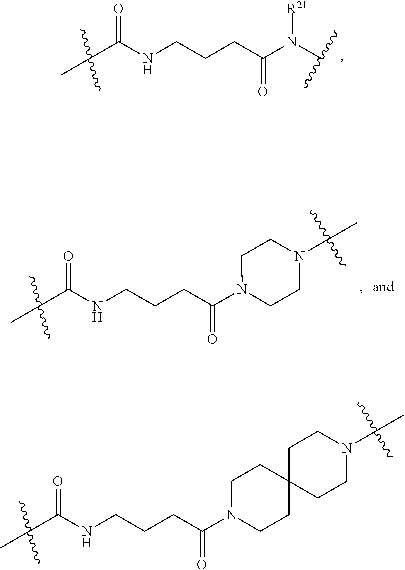

or a tautomer thereof, or a salt thereof.

Clause 227. The compound of any one of clauses 201 to 126, wherein A¹ of ring system A is independently —N═CR³—, —CR³═N—, or —CR³═CR³—.

Clause 228. The compound of clause 227, wherein A is of formula (IIA):

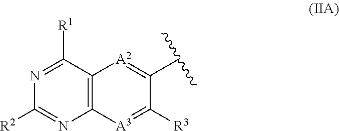

or a tautomer thereof, or a salt thereof, wherein:
A² is selected from N, and CR³;
A³ is independently selected from N, and CR²¹.

Clause 229. The compound of clause 228, wherein A² and A³ are each N.

Clause 230. The compound of clause 228, wherein A² and A³ are each independently CR³.

Clause 231. The compound of any one of clauses 227 to 230, wherein each R³ is H.

Clause 232. The compound of any one of clauses 227 to 231, wherein R² is —NH₂.

Clause 233. The compound of any one of clauses 227 to 231, wherein R² is optionally substituted (C₁-C₆)alkyl.

Clause 234. The compound of any one of clauses 227 to 231, wherein R² is —CH₃ or —CH₂OH.

Clause 235. The compound of any one of clauses 227 to 234, wherein R¹ is OH.

Clause 236. The compound of any one of clauses 227 to 235, wherein A is selected from:

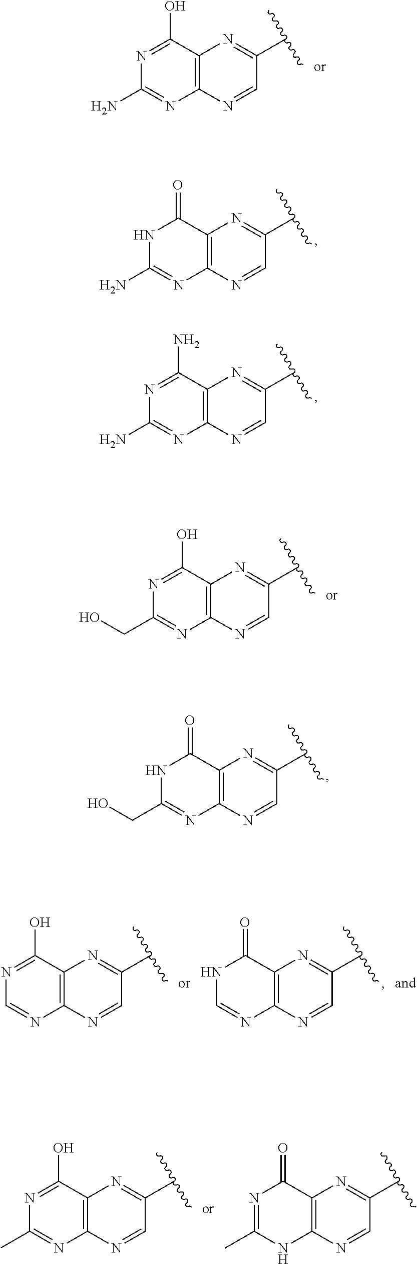

or a tautomer thereof.

Clause 237. The compound of any one of clauses 201 to 226, wherein A¹ of ring system A is NR²¹, S, O, or C(R²)₂.

Clause 238. The compound of clause 237, wherein A is of formula (IIB) or (IIC):

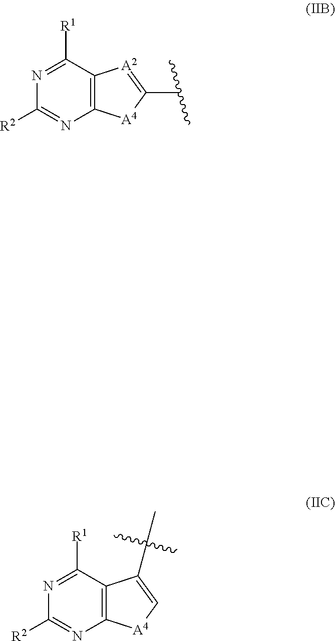

or a tautomer thereof, or a salt thereof, wherein A⁴ is selected from NR²¹, S, and O.

Clause 239. The compound of clause 238, wherein A⁴ is NR²¹.

Clause 240. The compound of clause 238 or 239, wherein A² is CR³.

Clause 241. The compound of any one of clauses 237 to 240, wherein R² is —NH₂.

Clause 242. The compound of any one of clauses 237 to 240, wherein R² is optionally substituted (C₁-C₆)alkyl (e.g., —CH₃ or —CH₂OH).

Clause 243. The compound of any one of clauses 237 to 242, wherein R¹ is OH.

Clause 244. The compound of any one of clauses 237 to 243, wherein A is:

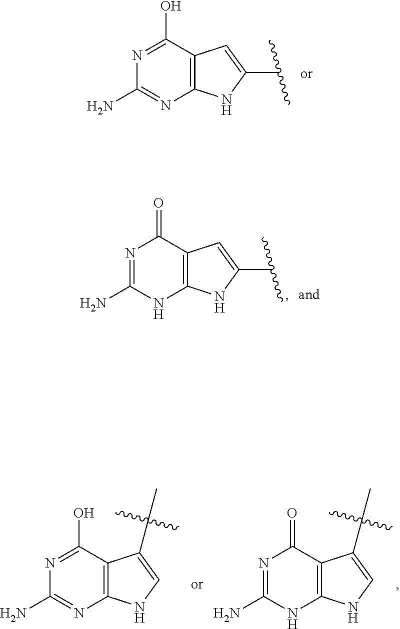 [SMILES: *c1cc2c(O)nc(N)nc2[nH]1 ; *c1cc2c(=O)nc(N)[nH]c2[nH]1 ; *c1c[nH]c2nc(N)nc(O)c12 ; *c1c[nH]c2[nH]c(N)nc(=O)c12]

or a tautomer thereof.

Clause 245. The compound of any one of clauses 227 to 244, wherein T¹ is CH₂.

Clause 246. The compound of any one of clauses 227 to 245, wherein Z¹ is NR²¹.

Clause 247. The compound of clause 246, wherein R²¹ is H.

Clause 248. The compound of clause 246, wherein R²¹ is methyl, ethyl, propyl, or propargyl.

Clause 249. The compound of any one of clauses 227 to 245, wherein Z¹ is O or S.

Clause 250. The compound of any one of clauses 227 to 245, wherein T¹-Z¹ is optionally substituted (C₁-C₆)alkylene.

Clause 251. The compound of clause 250, wherein T¹-Z¹ is —CH₂CH₂—.

Clause 252. The compound of clause 250, wherein T¹-Z¹ is —CH₂CH₂CH₂CH₂—or —CH₂CH₂CH₂—.

Clause 253. The compound of any one of clauses 201 to 252, wherein B is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, optionally substituted indole, and optionally substituted bicycloalkyl (e.g., bicyclo[1.1.1]pentane).

Clause 254. The compound of clause 253, wherein B is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

Clause 255. The compound of clause 253 or 254, wherein —B—Z²—is selected from:

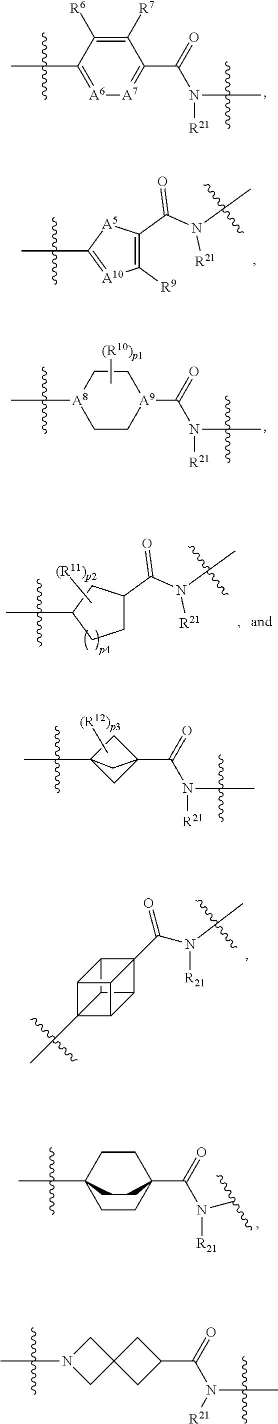

wherein:
A⁵ is selected from NR²¹, S, O, C(R⁵)₂;
A⁶-A⁹ are independently selected from N, and CR⁵; R²¹ is selected from H, and optionally substituted (C₁-C₆)alkyl;
A¹⁰ is selected from N, and CR;
each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

Clause 256. The compound of clause 255, wherein B—Z² is:

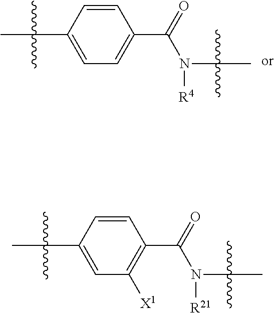

wherein X¹ is halogen.

Clause 257. The compound of clause 250, wherein A-T¹-Z¹—B—is selected from one of the following:

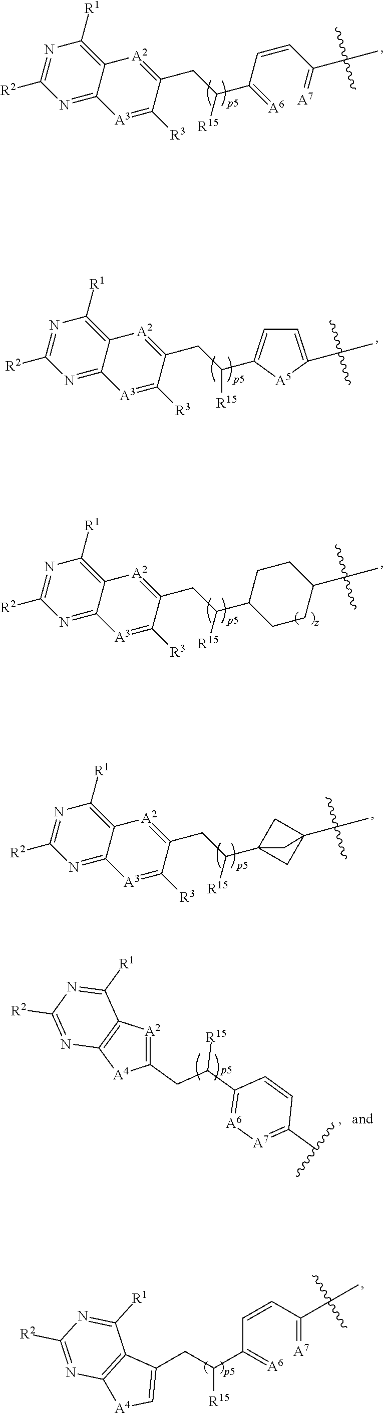

wherein:
A⁵ is selected from NR²¹, S, O, C(R⁵)₂;
A⁶ and A⁷ are independently selected from N, and, CR⁵;
z is 0 to 3
each R⁵ and R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

Clause 258. The compound of clause 201, wherein the compound comprises a cell surface folate receptor ligand selected from:

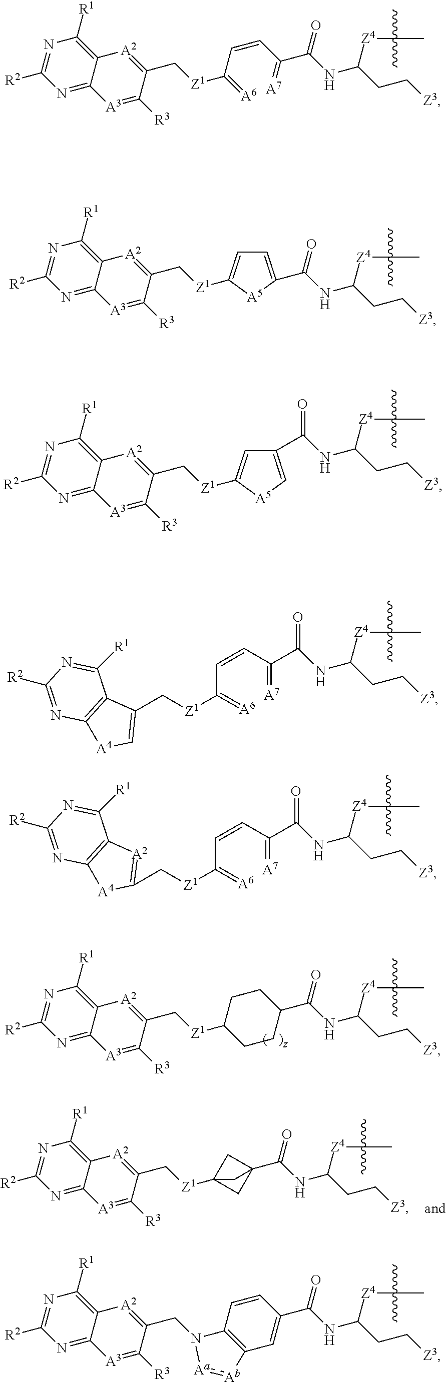

wherein:
A⁵ is selected from NR²¹, S, O, C(R⁵)₂;
A⁶ and A⁷ are independently selected from N, and, CR;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

Clause 259. The compound of clause 201, wherein the compound comprises a cell surface folate receptor ligand selected from:

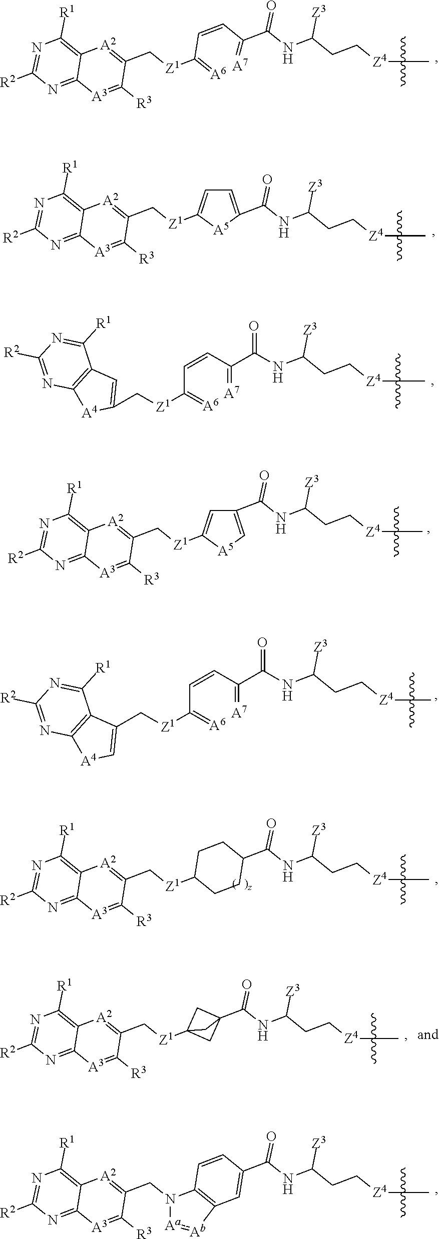

wherein:
A⁵ is selected from NR²¹, S, O, C(R⁵)₂;
A⁶ and A⁷ are each independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

Clause 260. The compound of any one of clauses 201 to 259, wherein n is 1.

Clause 261. The compound of any one of clauses 201 to 259, wherein n is at least 2.

Clause 262. The compound of clause 261, wherein n is 2 to 20 (e.g., n is 2 to 6, such as 2 or 3).

Clause 263. The compound of any one of clauses 201 to 262, wherein L comprises a backbone of at least 10 consecutive atoms (e.g., by a backbone of at least 12, at least 14, or at least 16 consecutive atoms, e.g., and wherein the backbone is up to 100 consecutive atoms).

Clause 264. The compound of any one of clauses 261 to 263, wherein L comprises one or more linking moieties independently selected from —C₁₋₆-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, —NH—, and —NMe—, wherein p is 1 to 10.

Clause 265. The compound of clause 263, wherein L comprises repeating ethylene glycol moieties (e.g., —CH₂CH₂O—or —OCH₂CH₂—).

Clause 266. The compound of clause 263 or 264, wherein L comprises 1 to 20 ethylene glycol moieties (e.g., 2 to 10, or 4 to 6 ethylene glycol moieties).

Clause 267. The compound of any one of clauses 201 to 266, wherein L is of formula (IV):

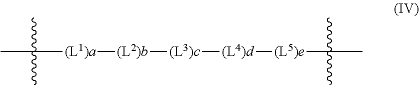

wherein
each L¹ to L⁵ is independently a linking moiety which together provide a linear or branched linker between Z⁴ and Y;
a is 1 or 2; and
b, c, d, and e are each independently 0, 1, or 2.

Clause 268. The compound of clause 267, wherein -(L¹)ₐ- comprises an optionally substituted alkyl or ethylene glycol linking moiety.

Clause 269. The compound of clause 267 or 268, wherein each L¹ is independently selected from: —C₁₋₆-alkylene-, —(CH₂CH₂O)ₜ—, —C₁₋₆-alkylene-NR⁴CO—, —C₁₋₆-alkyleneCONH—, or OCH₂, wherein t is 1 to 20; and R⁴ is independently selected from H, and optionally substituted (C₁-C₆)alkyl.

Clause 270. The compound of any one of clauses 267 to 269, wherein:

each L² is independently selected from —NR⁴CO—C₁₋₆-alkylene-, —CONR⁴—C₁₋₆-alkylene,

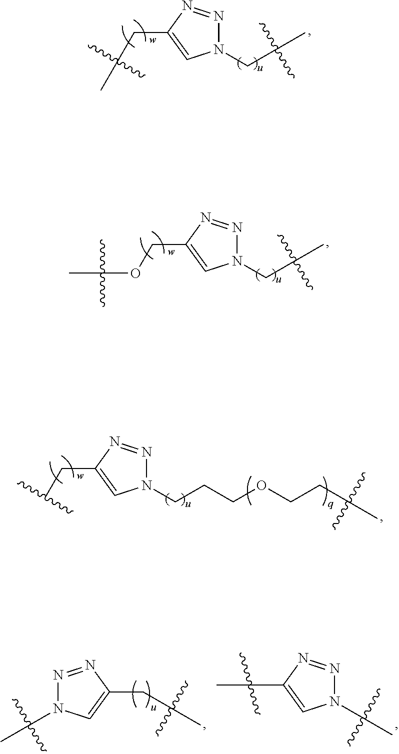

—OCH₂—, and —(OCH₂CH₂)_{q}—, wherein q is 1 to 10, u is 0 to 10, w is 1 to 10, and R⁴ is independently selected from H, and optionally substituted (C₁-C₆)alkyl; and
each L⁴ is absent or independently selected from —C₁₋₆-alkylene-, —(CH₂CH₂O)ₜ—, —C₁₋₆-alkylene-NHCO—, —C₁₋₆-alkyleneCONH—, or OCH₂,
wherein t is 1 to 20.

Clause 271. The compound of any one of clauses 267 to 270, wherein when n is 2 or more, at least one L³ is present and is a branched linking moiety.

Clause 272. The compound of any one of clauses 267 to 271, wherein each L³ is independently selected from:

—OCH₂CH₂—,

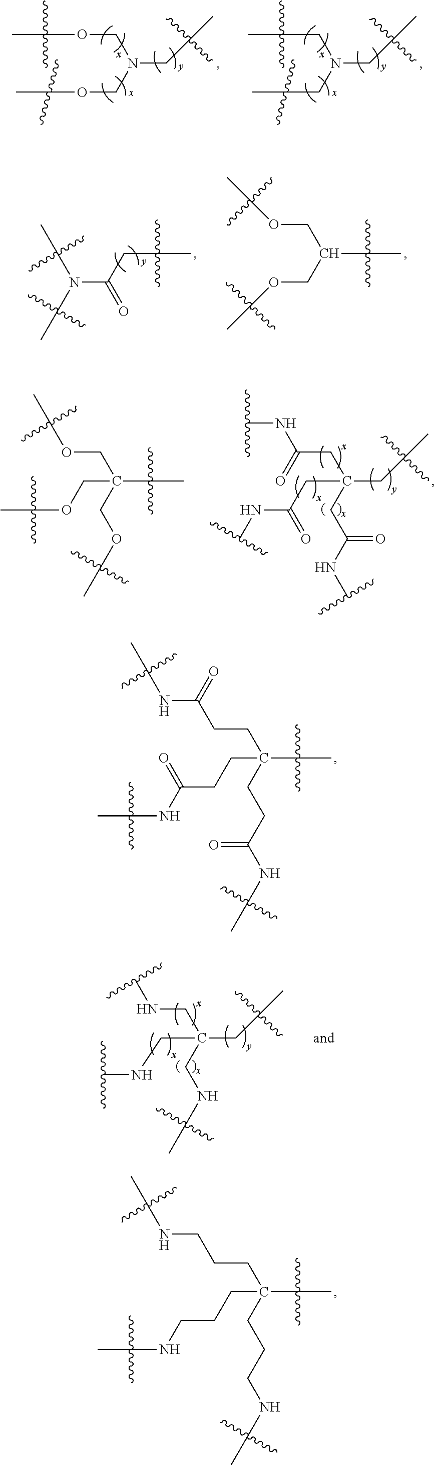

wherein each x and y are each independently 1 to 10.

Clause 273. The compound of any one of clauses 262 to 272, wherein:
each L⁵ is independently —CH₂O—; —(CH₂CH₂O)ₜ—, —NR⁴CO—, —C₁₋₆-alkylene-,

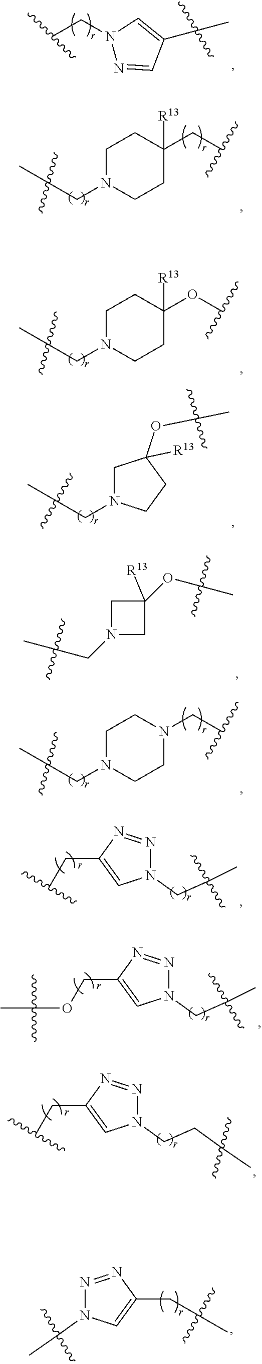

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
   each r independently 0 to 20.

Clause 274. The compound of any one of clauses 267 to 273, wherein a is 1.

Clause 275. The compound of any one of clauses 267 to 274, wherein at least one of b, c, d, and e is not 0.

Clause 276. The compound of any one of clauses 267 to 275, wherein b, d, and e are each independently 1 or 2.

Clause 277. The compound of any one of clauses 267 to 276, wherein a, b, d, and e are each 1, and c is 0.

Clause 278. The compound of any one of clauses 267 to 277, wherein the linker L is selected from any one of the structures of Table 3.

Clause 279. The compound of any one of clauses 201 to 278, wherein the compound comprises a cell surface folate receptor ligand of one of the structures of Tables 1 or 2.

Clause 280. The compound of any one of clauses 201 to 279, wherein Y is selected from small molecule, dye, fluorophore, monosaccharide, polysaccharide (e.g., disaccharide, or trisaccharide), lipid, enzyme, enzyme substrate and chemoselective ligation group or precursor thereof.

Clause 281. The compound of any one of clauses 201 to 280, wherein Y is a moiety that specifically binds an extracellular target protein.

Clause 282. The compound of clause 281, wherein the target protein is a membrane bound protein.

Clause 283. The compound of clause 281, wherein the target protein is a soluble extracellular protein.

Clause 284. The compound of any one of clauses 201 to 283, wherein Y is a target-binding small molecule.

Clause 285. The compound of clause 284, wherein Y is a small molecule inhibitor or ligand of the target protein.

Clause 286. The compound of clause 285, wherein Y is an allosteric desymmetrization TNFα inhibitor.

Clause 287. The compound of any one of clauses 201 to 285, wherein Y is a target-binding biomolecule.

Clause 288. The compound of clause 187, wherein the biomolecule is selected from peptide, protein, glycoprotein, polynucleotide, aptamer, and antibody or antibody fragment.

Clause 289. The compound of clause 188, wherein Y is selected from antibody, antibody fragment (e.g., antigen-binding fragment of an antibody), chimeric fusion protein, an engineered protein domain, and D-protein binder of target protein.

Clause 290. The compound of clause 189 wherein Y is antibody or antibody fragment that specifically binds the target protein and the compound is of formula (VIIIa):

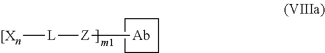

or a pharmaceutically acceptable salt thereof, wherein:
n is 1 to 20;
m1 is an average loading of 1 to 80;
   each X is a moiety that binds to a cell surface folate receptor;
each L is a linker;
each Z is a residual moiety resulting from the covalent linkage of a chemoselective ligation group to a compatible group of Ab; and
Ab is the antibody or antibody fragment that specifically binds the target protein.

Clause 291. The compound of clause 290, wherein X is not folic acid, methotrexate, or pemetrexed.

Clause 292. The compound of any one of clauses 290 to 291, wherein X is selected from:

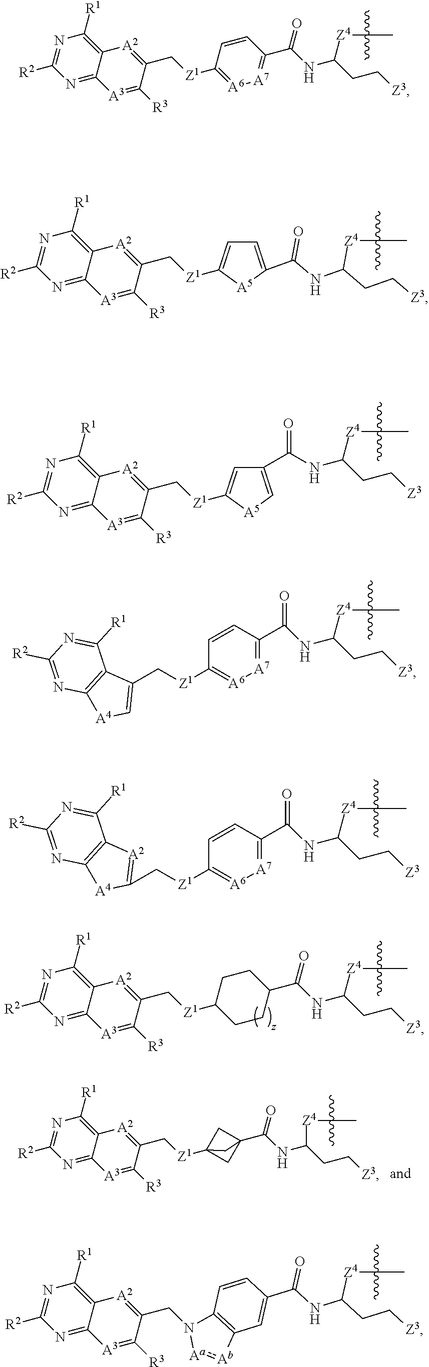

wherein:
A⁵ is selected from NR²¹, S, O, C(R⁵)₂;
A⁶ and A⁷ are independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹;
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

Clause 293. The compound of any one of clauses 290 to 291, wherein X is selected from:

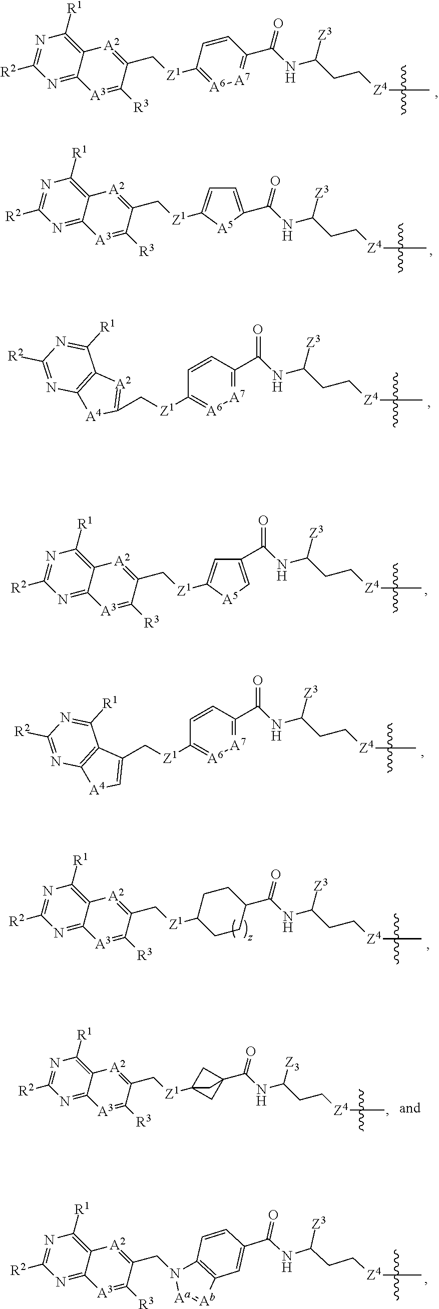

wherein:
A⁵ is selected from NR, S, O, C(R⁵)₂;
A⁶ and A⁷ are each independently selected from N, and, CR⁵;
z is 0 to 3;
[symbol] is a single bond or a double bond;
wherein when [symbol] is a single bond A^{a} is selected from C(R⁵)₂, and C═O, and A^{b} is selected from C(R⁵)₂, and NR²¹;
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵. The compound of any one of clauses 85 to 88, wherein n is 1 to 6.

Clause 294. The compound of clause 293, wherein n is 2 or less.

Clause 295. The compound of clause 294, wherein n is 1.

Clause 296. The compound of any one of clauses 291 to 294, wherein n is at least 2.

Clause 297. The compound of clause 296, wherein n is 2.

Clause 298. The compound of clause 296, wherein n is 3.

Clause 299. The compound of clause 296, wherein n is 4.

Clause 300. The compound of any one of clauses 291 to 299, wherein m1 is 1 to 20.

Clause 301. The compound of clause 300, wherein m1 is 1 to 12.

Clause 302. The compound of clause 300 or 301, wherein m1 is at least about 2.

Clause 303. The compound of clause 300 or 301, wherein m1 is at least about 3.

Clause 304. The compound of clause 300 or 301, wherein m1 is at least about 4.

Clause 305. The compound of any one of clauses 291 to 304, wherein Z is a residual moiety resulting from the covalent linkage of a thiol-reactive chemoselective ligation group to one or more cysteine residue(s) of Ab.

Clause 306. The compound of any one of clauses 291 to 304, wherein Z is a residual moiety resulting from the covalent linkage of an amine-reactive chemoselective ligation group to one or more lysine residue(s) of Ab.

Clause 307. The compound of any one of clauses 291 to 306, wherein the antibody or antibody fragment is an IgG antibody.

Clause 308. The compound of any one of clauses 291 to 106, wherein the antibody or antibody fragment is a humanized antibody.

Clause 309. The compound of any one of clauses 291 to 308, wherein the antibody or antibody fragment specifically binds to a secreted or soluble protein.

Clause 310. The compound of any one of clauses 291 to 308, wherein the antibody or antibody fragment specifically binds to a cell surface receptor.

Clause 311. A method of internalizing a target protein in a cell comprising a cell surface folate receptor, the method comprising: contacting a cellular sample comprising the cell and the target protein with an effective amount of a compound according to any one of clauses 201 to 310, wherein the compound specifically binds the target protein and specifically binds the cell surface folate receptor to facilitate cellular uptake of the target protein.

Clause 312. The method of clause 311, wherein the target protein is a membrane bound protein.

Clause 313. The method of clause 311, wherein the target protein is an extracellular protein.

Clause 314. The method of any one of clauses 311 to 313, wherein the compound or conjugate comprises an antibody or antibody fragment (Ab) that specifically binds the target protein.

Clause 315. A method of reducing levels of a target protein in a biological system, the method comprising: contacting the biological system with an effective amount of a compound according to any one of clauses 201 to 310, wherein the compound specifically binds the target protein and specifically binds a cell surface receptor of cells in the biological system to facilitate cellular uptake and degradation of the target protein.

Clause 316. The method of clause 315, wherein the biological system comprises cells that comprise a folate cell surface receptor.

Clause 317. The method of clause 315 or 316, wherein the biological system is a human subject.

Clause 318. The method of any one of clauses 315 to 317, wherein the biological system is an in vitro cellular sample.

Clause 319. The method of any one of clauses 315 to 318, wherein the target protein is a membrane bound protein.

Clause 320. The method of any one of clauses 315 to 318, wherein the target protein is an extracellular protein.

Clause 321. A method of treating a disease or disorder associated with a target protein, the method comprising: administering to a subject in need thereof an effective amount of a compound according to any one of clauses 201 to 210, wherein the compound specifically binds the target protein.

Clause 322. The method of clause 321, wherein the disease or disorder is an inflammatory disease.

Clause 323. The method of clause 321, wherein the disease or disorder is an autoimmune disease.

Clause 324. The method of clause 321, wherein the disease or disorder is a cancer.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 6. EXAMPLES

The following examples are offered to illustrate the present disclosure and are not to be construed in any way as limiting the scope of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### 6.1. Preparation of Compounds

The following are illustrative schemes and examples of how the compounds described herein can be prepared and tested. Although the examples can represent only some embodiments, it should be understood that the following examples are illustrative and not limiting. All substituents, unless otherwise specified, are as previously defined. The reagents and starting materials are readily available to one of ordinary skill in the art. The specific synthetic steps for each of the routes described may be adapted and combined in different ways, or in conjunction with steps from different schemes, to prepare the compounds described herein.

### 6.1.1. Example 1: Compound I-1

### Synthesis of (S)-38-(4-(((2-amino-4-oxo-3,4-dihydropteridin-6-yl)methyl)amino)benzamido)-1-(2,5-dioxo-2,5-dihydro-1H-pyrrol-1-yl)-3,35-dioxo-7,10,13,16,19,22,25,28,31-nonaoxa-4,34-diazanonatriacontan-39-oic acid (Compound I-1)

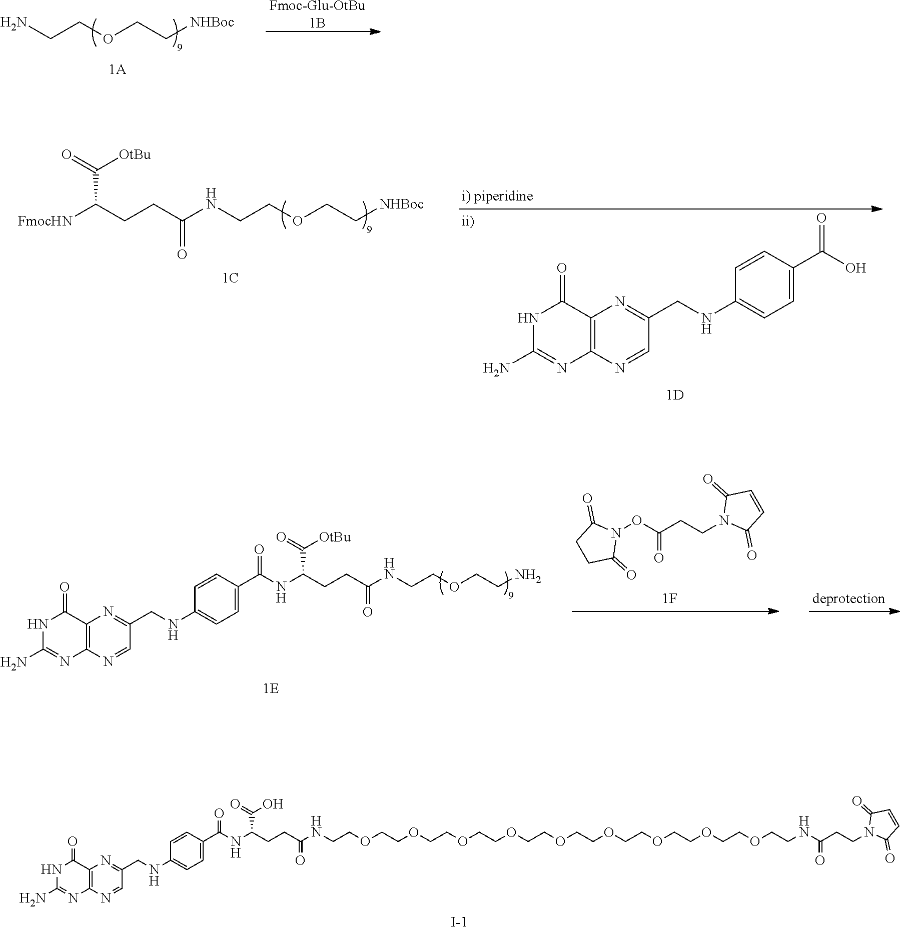

To a solution of Compound 1A and Fmoc-Glu-OtBu (1B3) in DMF is added DIPEA, HOBt and HBTU in DMF. The reaction mixture is stirred at room temperature and monitored by TLC until ninhydrin test shows no free amine is observed. Upon completion, DMF is removed under vacuum and the reaction mixture is purified by preparatory HPLC. Fractions containing the desired product were combined and lyophilized to dryness to afford Compound 1C.

To a solution of Compound 1C in DMF is added piperidine, and the reaction mixture is stirred at room temperature for 1 h. A mixture of pteroic acid (1D), DIPEA, HOBt and HBTU in DMF/DMSO is added to the solution of Compound 1C. The reaction is monitored by LCMS until no free amine is observed. The reaction mixture is purified by preparatory HPLC. Fractions containing the coupled product were combined and lyophilized to dryness. The protected intermediate in DCM is treated with excess TFA and stirred at room temperature until deprotected. The mixture is concentrated to dryness to afford Compound 1E.

To a stirred solution of Compound 1E and Compound 1F in DMF, DIPEA is added and reaction mixture is stirred for 3 h. The progress of reaction is monitored by LC-MS and stirred until completion. The reaction mixture is purified by preparatory HPLC to afford Compound I-1.

### 6.1.2. Example 2: Compound I-2

### Synthesis of (S)-40-(4-(((2-amino-4-oxo-3,4-dihydropteridin-6-yl)methyl)amino)benzamido)-1,31,37-trioxo-1-(perfluorophenoxy)-4,7,10,13,16,19,22,25,28-nonaoxa-32,36-diazahentetracontan-41-oic acid (Compound I-2)

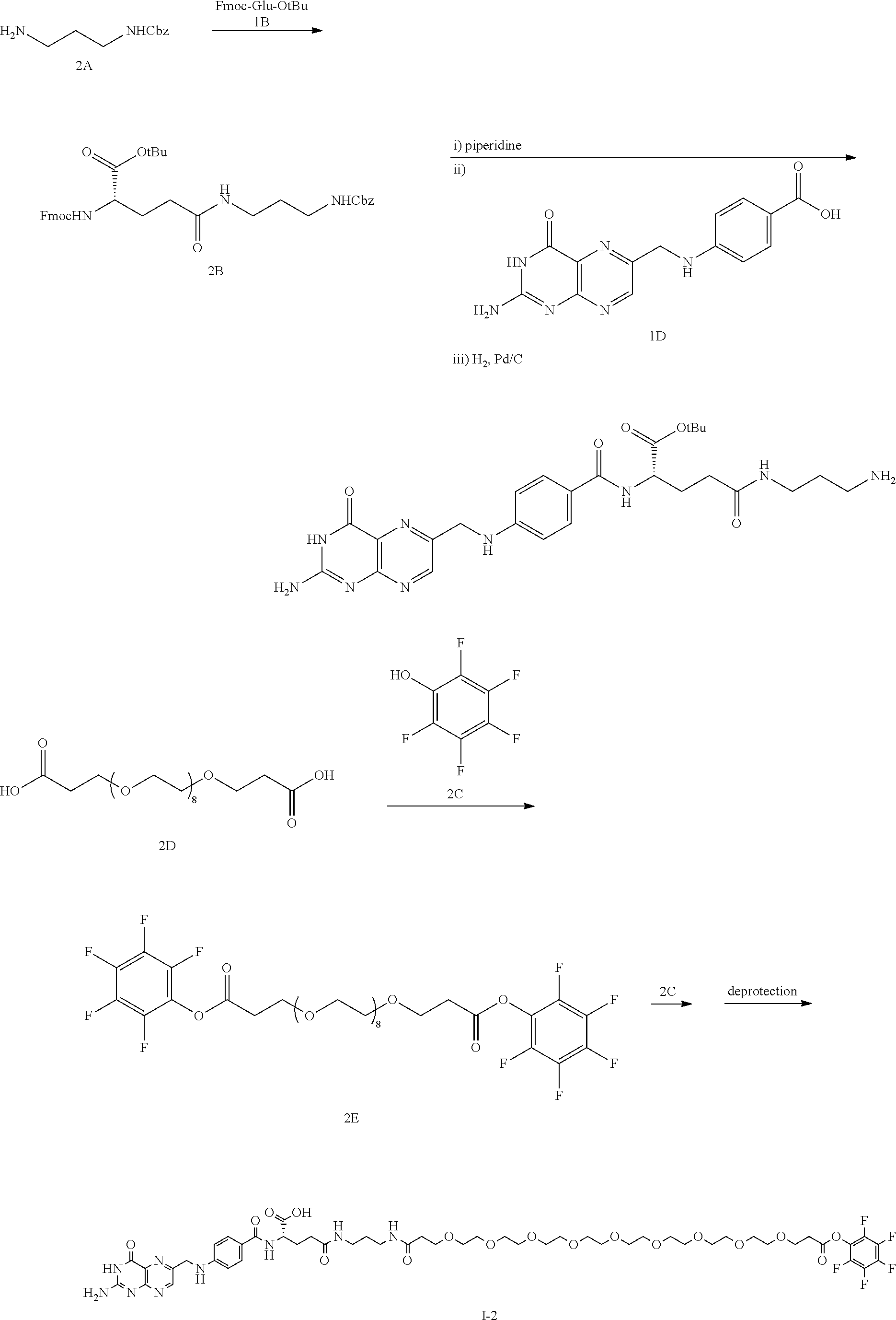

To a solution of Compound 2A and Fmoc-Glu-OtBu (1B) in DMF is added DIPEA, HOBt and HBTU in DMF. The reaction mixture is stirred at room temperature and monitored by TLC until ninhydrin test shows no free amine is observed. Upon completion, DMF is removed under vacuum and the reaction mixture is purified by preparatory HPLC. Fractions containing the desired product are combined and lyophilized to dryness to afford Compound 2B.

To a solution of Compound 2B in DMF is added piperidine, and the reaction mixture is stirred at room temperature for 1 h. A mixture of -pteroic acid (1D), DIPEA, HOBt and HBTU in DMF is added to the solution of Compound 1C. The reaction is monitored by LCMS until no free amine is detected. Upon completion, the reaction mixture is purified by preparatory HPLC. Fractions containing the desired product are combined and lyophilized to dryness. The protected intermediate is dissolved in EtOH/EtOAc and Pd/C is added and the mixture placed under a hydrogen atmosphere and stirred until the Cbz group is removed. The mixture is filtered and concentrated, and the crude material is purified by preparative HPLC to afford Compound 2C.

A solution of Compound 2D and pentafluorophenol in ethyl acetate is cooled at 0° C., N,N′-diisopropylcarbodiimide is added and reaction mixture is stirred at room temperature for 3 h. Reaction mixture is filtered through Celite bed and Celite bed was washed with ethyl acetate. The filtrate is concentrated to get crude product which is purified by column chromatography using silica gel (100-200 mesh) and 0-10% ethyl acetate in hexane to afford Compound 2E.

To a stirred solution of Compound 2C and Compound 2E in DMF, DIPEA is added and reaction mixture is stirred for 3 h. The progress of reaction was monitored by LC-MS. After the completion of reaction, a 95:2.5:2.5 mixture of TFA:TIPS:H₂O is added to the reaction mixture and the reaction mixture is stirred at room temperature for 30 min. The reaction mixture is concentrated under reduced pressure to afford crude. The crude is purified by preparatory HPLC to afford Compound I-2.

### 6.1.3. Example 3: Compounds Prepared from Intermediate 3C

### Synthesis of N²-(4-(((2-amino-4-oxo-3,4-dihydropteridin-6-yl)methyl)amino)benzoyl)-N⁵-(4-(1-(2-(3-oxo-3-(perfluorophenoxy)propoxy)ethyl)-1H-1,2,3-triazol-4-yl)butyl)-L-glutamine (Compound I-3)

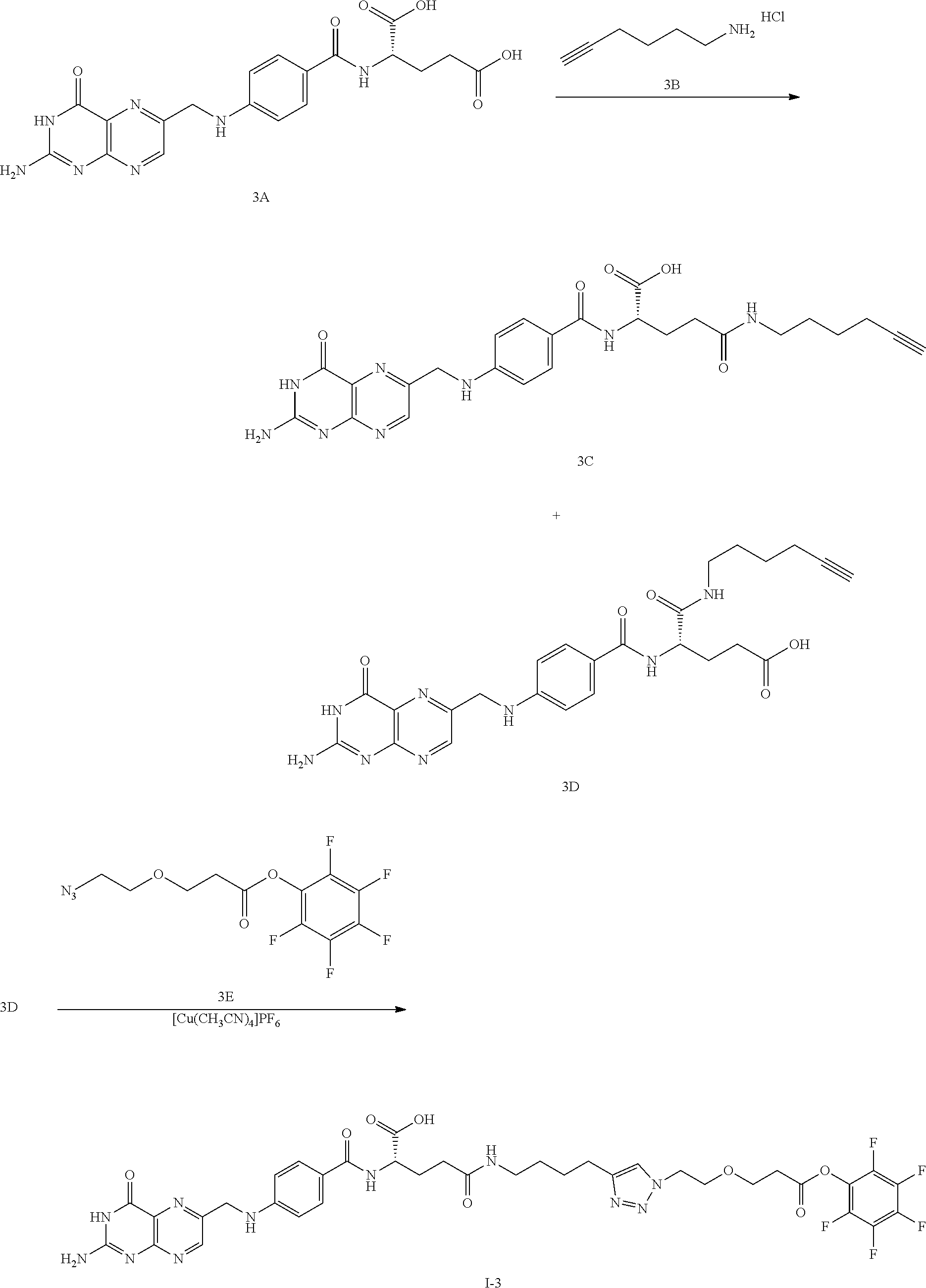

To a solution of (4-(((2-amino-4-oxo-3,4-dihydropteridin-6-yl)methyl)amino)benzoyl)-L-glutamic acid (3A) (1.0 eq, 1.0 g, 2.27 mmol) in N,N-dimethylformamide (20 mL) and dimethyl sulfoxide (20 mL), N-hydroxysuccinimide (1.1 eq, 0.287 g, 2.49 mmol), 1-(3-Dimethylaminopropyl)-3-ethylcarbodiimide hydrochloride (1.1 eq, 0.478 g, 2.49 mmol) and N,N-diisopropylethylamine (3.0 eq, 1.25 mL, 6.80 mmol) were added and reaction mixture was stirred at room temperature for 30 minutes. Then, hex-5-yn-1-amine hydrochloride (3B) (1.1 eq., 0.333 g, 2.49 mmol) was added and reaction mixture was stirred at room temperature for 16 h. After completion, reaction mixture was poured into 30% acetone in diethyl ether to get solid which was filtered off and dried to afford crude. Crude was purified by prep HPLC (22-35% acetonitrile in water with 0.1% TFA). Fractions containing the desired product were combined and lyophilized to dryness to afford a mixture of Compounds 3C and 3D, which was repurified by prep HPLC (20-40% acetonitrile in water with 0.1% acetic acid). Fractions containing the desired product were combined and lyophilized to dryness to afford Compound 3C as a yellow solid, Yield: 0.080 g, 6.72%; LC-MS m/z 521.0 [M+1]⁺ and Compound 3D as a yellow solid, Yield: 0.040 g, 2.78%; LC-MS m/z 521.0 [M+1]⁺.

To a solution of Compound 3C (1.0 eq., 0.025 g, 0.048 mmol) in dimethylsulfoxide (0.5 mL), perfluorophenyl 3-(2-azidoethoxy)propanoate (3E) (1.0 eq., 0.015 g, 0.048 mmol) was added and stirred for 5 minutes. Then, tetrakis(acetonitrile)copper(I) hexafluorophosphate (2.8 eq., 0.050 g, 0.134 mmol) was added and reaction mixture was stirred at room temperature for 30 minutes. After completion, reaction mixture was diluted with acetonitrile and purified by prep HPLC (40-55% acetonitrile in water with 0.1% acetic acid). Fractions containing the desired product were combined and lyophilized to dryness to afford Compound I-3 as a yellow solid. Yield: 0.0088 g, 20.7%; LC-MS m/z 846.18 [M+1]⁺; ¹H NMR (400 MHz, DMSO-d₆) δ12.51 (bs, 1H), 11.42 (bs, 1H), 8.64 (s, 1H), 8.19 (d, J=8.0 Hz, 1H), 7.81 (t, J=5.6 Hz, 1H), 7.72 (s, 1H), 7.64 (d, J=8.4 Hz, 2H), 6.94 (bs, 3H), 6.63 (d, J=8.0 Hz, 2H), 4.48-4.44 (m, 4H), 4.25 (bs, 1H), 3.81 (t, J=5.6 Hz, 2H), 3.74 (t, J=6.0 Hz, 2H), 3.01 (t, J=5.2 Hz, 4H), 2.56-2.54 (m, 2H), 2.16-2.14 (m, 2H), 2.03-2.02 (m, 1H), 1.91-1.87 (m, 1H), 1.54-1.48 (m, 2H), 1.41-1.35 (m, 2H).

Compounds I-4 to I-8 were prepared by reacting intermediate compound 3C with an appropriate azide containing intermediate by adapting the general method described above for compound I-3. Table X outlines the data for each of the compounds that were synthesized according to the general method outlined for the synthesis of compound I-3.

TABLE X
Data for compounds synthesized from intermediate 3C

| # | Compound structure, name and Data |
|---|---|
| I-4 | 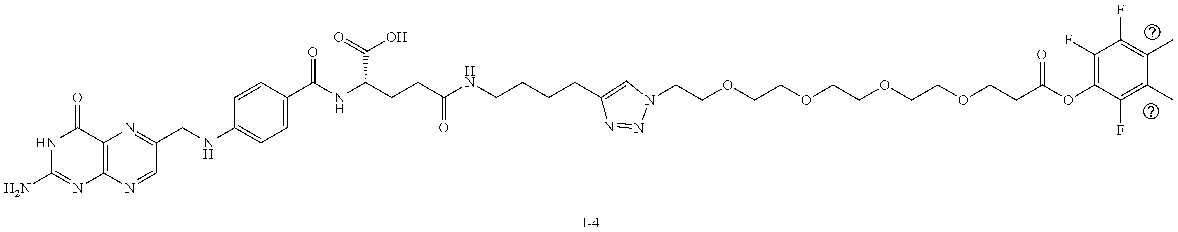 |
| | N²-(4-(((2-amino-4-oxo-3,4-dihydropteridin-6-yl)methyl)amino)benzoyl)-N⁵-(4-(1-(15- |
| | oxo-15-(perfluorophenoxy)-3,6,9,12-tetraoxapentadecyl)-1H-1,2,3-triazol-4-yl)butyl)-L- |
| | glutamine |
| | LC-MS: m/z 978.25 [M + 1]⁺ |
| | ¹H NMR (400 MHz, DMSO-d₆): δ 12.44 (bs, 1H), 11.42 (bs, 1H), 8.66 (s, 1H), 8.20 (d, J = 7.6 |
| | Hz, 1H), 7.82-7.80 (m, 1H), 7.78 (s, 1H), 7.64 (d, J = 8.0 Hz, 2H), 7.12 (bs, 2H), 6.63 (d, J = |
| | 8.4 Hz, 2H), 4.49 (s, 2H), 4.43 (t, J = 5.2 Hz, 2H), 4.26-4.23 (m, 2H), 3.78-3.73 (m, 4H), 3.52- |
| | 3.47 (m, 11H), 3.05-2.99 (m, 4H), 2.58-2.55 (m, 2H), 2.16-2.15 (m, 2H), 2.07-2.02 (m, 1H), |
| | 1.93-1.85 (m, 2H), 1.56-1.50 (m, 2H), 1.40-1.37 (m, 2H) |
| I-5 | 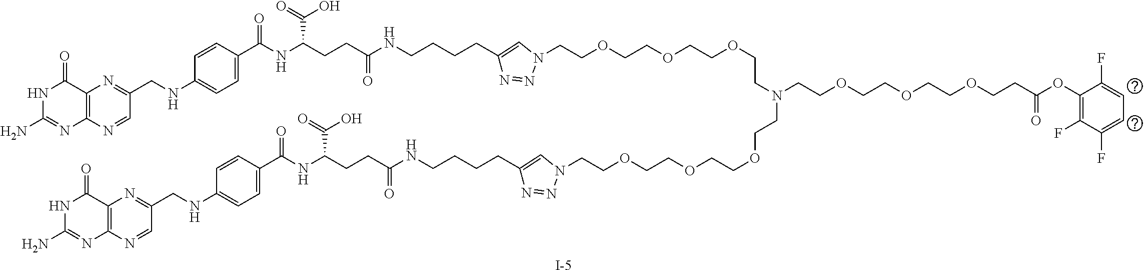 |
| | (2S,2′S)-5,5′-((((12-(2-(2-(2-(3-oxo-3-(perfluorophenoxy)propoxy)ethoxy)ethoxy)ethyl)- |
| | 3,6,9,15,18,21-hexaoxa-12-azatricosane-1,23-diyl)bis(1H-1,2,3-triazole-1,4- |
| | diyl))bis(butane-4,1-diyl))bis(azanediyl))bis(2-(4-(((2-amino-4-oxo-3,4-dihydropteridin-6- |
| | yl)methyl)amino)benzamido)-5-oxopentanoic acid) |
| | LCMS m/z 1830.85 [M + 1]⁺ |
| | ¹H NMR (400 MHz, DMSO-d₆ with D₂O exchange) δ 8.62 (s, 2H), 7.73 (s, 2H), 7.61 |
| | (d, J = 8.4 Hz, 2H), 6.62 (d, J = 8.8 Hz, 2H), 4.47 (s, 4H), 4.40 (bs, 4H), 4.26-4.23 (m, |
| | 2H), 3.76-3.68 (m, 10H), 3.49-3.48 (m, 26H), 3.44-3.42 (m, 6H), 3.02-3.00 (m, 4H), |
| | 2.98-2.93 (m, 2H), 2.50 (bs, 4H), 2.18-2.14 (m, 4H), 2.10-2.00 (m, 2H), 1.91-1.89 (m, |
| | 2H), 1.52-1.48 (m, 4H), 1.46-1.33 (m, 4H) |
| I-6 | 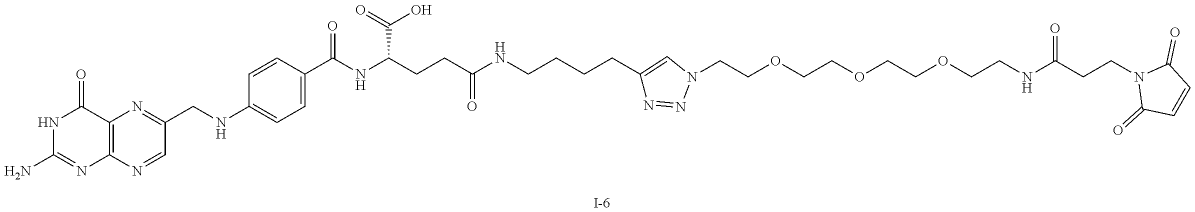 |
| | N²-(4-(((2-amino-4-oxo-3,4-dihydropteridin-6-yl)methyl)amino)benzoyl)-N⁵-(4-(1-(15- |
| | (2,5-dioxo-2,5-dihydro-1H-pyrrol-1-yl)-13-oxo-3,6,9-trioxa-12-azapentadecyl)-1H-1,2,3- |
| | triazol-4-yl)butyl)-L-glutamine |
| | LC-MS: m/z 890.32 [M + 1]⁺ |
| | ¹H NMR (400 MHz, DMSO-d₆): δ 12.5 (bs, 1H), 11.5 (bs, 1H), 8.64 (s, 1H), 8.20 (d, J = 7.44 |
| | Hz, 1H), 8.03 (t, J = 5.32 Hz, 1H), 7.82 (t, J = 5.84 Hz, 1H), 7.78 (s, 1H), 7.64 (d, J = 8.72 Hz, |
| | 2H), 6.99 (s, 2H), 6.63 (d, J = 8.68 Hz, 2H), 4.48 (s, 2H), 4.44 (t, J = 5.4 Hz, 2H), 4.30-4.20 |
| | (m, 1H), 3.77 (t, J = 5.16 Hz, 2H), 3.59-3.45 (m, 10H), 3.34 (t, J = 5.92 Hz, 2H), 3.15-3.12 (m, |
| | 2H), 3.03-3.00 (m, 2H), 2.57 (t, J = 7.6 Hz, 2H), 2.33-2.31 (m, 2H), 2.17-2.15 (m, 2H), 2.10- |
| | 2.00 (m, 1H), 1.90-1.80 (m, 1H), 1.56-1.52 (m, 2H), 1.40-1.36 (m, 2H) |
| I-7 | 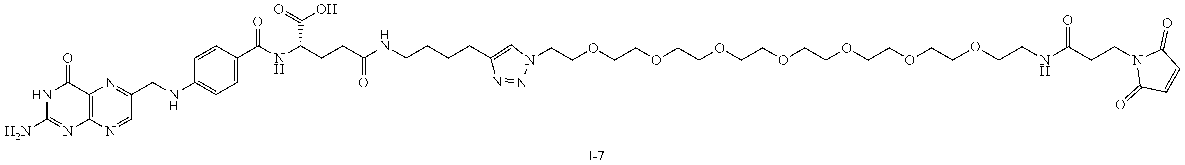 |
| | N²-(4-(((2-amino-4-oxo-3,4-dihydropteridin-6-yl)methyl)amino)benzoyl)-N⁵-(4-(1-(27- |
| | (2,5-dioxo-2,5-dihydro-1H-pyrrol-1-yl)-25-oxo-3,6,9,12,15,18,21-heptaoxa-24- |
| | azaheptacosyl)-1H-1,2,3-triazol-4-yl)butyl)-L-glutamine |
| | LCMS m/z 1066.34 [M + 1]⁺ |
| | ¹H NMR (400 MHz, DMSO-d₆) δ 12.44 (bs, 1H), 8.66 (s, 1H), 8.19 (d, J = 7.2 Hz, |
| | 1H), 8.01 (t, J = 4.8 Hz, 1H), 7.81 (t, J = 5.2 Hz, 1H), 7.78 (s, 1H), 7.64 (d, J = 8.8 Hz, |
| | 2H), 7.20-7.07 (m, 2H), 6.99 (s, 2H), 6.63 (d, J = 8.8 Hz, 2H), 4.50 (s, 2H), 4.44 (t, J = |
| | 5.2 Hz, 2H), 4.30-4.20 (m, 1H), 3.77 (t, J = 5.2 Hz, 2H), 3.60-3.47 (m, 26H), 3.35 (t, J = |
| | 6.0 Hz, 2H), 3.16-3.13 (m, 2H), 3.03-3.02 (m, 2H), 2.57 (t, J = 7.2 Hz, 2H), 2.34- |
| | 2.30 (m, 2H), 2.17-2.15 (m, 2H), 2.10-2.00 (m, 1H), 1.90-1.80 (m, 1H), 1.56-1.52 (m, |
| | 2H), 1.40-1.36 (m, 2H) |
| I-8 | 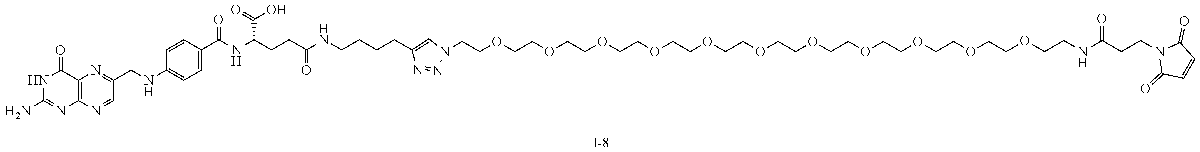 |
| | N²-(4-(((2-amino-4-oxo-3,4-dihydropteridin-6-yl)methyl)amino)benzoyl)-N⁵-(4-(1-(39- |
| | (2,5-dioxo-2,5-dihydro-1H-pyrrol-1-yl)-37-oxo-3,6,9,12,15,18,21,24,27,30,33-undecaoxa- |
| | 36-azanonatriacontyl)-1H-1,2,3-triazol-4-yl)butyl)-L-glutamine |
| | LCMS m/z 1242.47 [M + 1]⁺ |
| | ¹H NMR (400 MHz, DMSO-d₆) δ 12.50 (bs, 1H), 8.64 (s, 1H), 8.15 (bs, 1H), 8.01 (t, J = |
| | 6.0 Hz, 1H), 7.83-7.81 (m, 1H), 7.78 (s, 1H), 7.63 (d, J = 8.8 Hz, 2H), 7.00 (s, 2H), |
| | 6.92 (t, J = 6.0 Hz, 2H), 6.63 (d, J = 8.8 Hz, 2H), 4.48-4.43 (m, 4H), 4.25 (bs, 1H), 3.77 |
| | (t, J = 5.2 Hz, 2H), 3.58 (t, J = 7.2 Hz, 2H), 3.49-3.47 (m, 40H), 3.37-3.35 (m, 2H), |
| | 3.14-3.13 (m, 2H), 3.03-3.02 (m, 2H), 2.59-2.55 (m, 2H), 2.34-2.30 (m, 2H), 2.20-2.15 |
| | (m, 2H), 2.10-2.00 (m, 1H), 1.90-1.80 (m, 1H), 1.56-1.52 (m, 2H), 1.41-1.37 (m, 2H). |

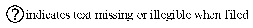

### 6.1.4. Example 4: Compound I-9

### Synthesis of (S)-4-(4-(((2-amino-4-oxo-3,4-dihydropteridin-6-yl)methyl)amino)benzamido)-5-oxo-5-((4-(1-(2-(3-oxo-3-(perfluorophenoxy)propoxy)ethyl)-1H-1,2,3-triazol-4-yl)butyl)amino)pentanoic acid (Compound I-9)

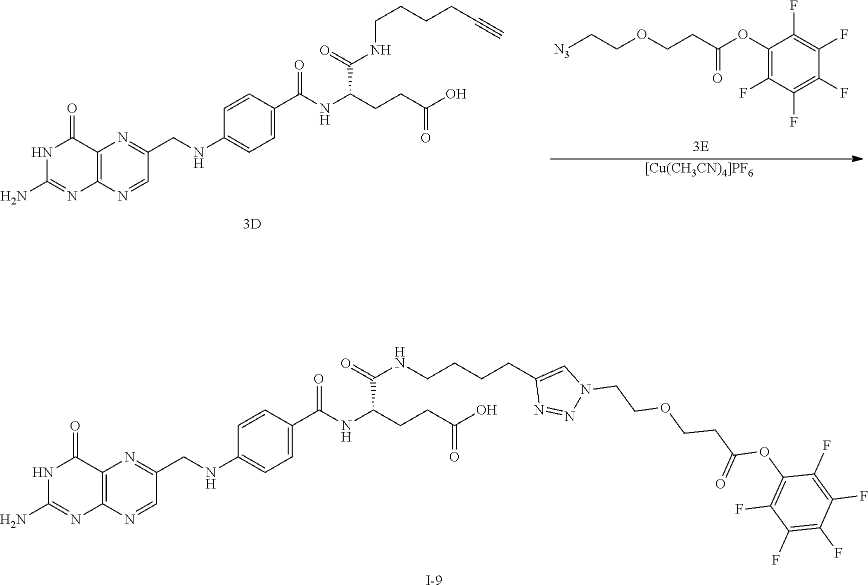

To a solution of Compound 3D (1.0 eq., 0.020 g, 0.038 mmol) in dimethylsulfoxide (0.5 mL), Compound 3E (1.0 eq., 0.012 g, 0.038 mmol) was added and stirred for 5 minutes. Then, tetrakis(acetonitrile)copper(I) hexafluorophosphate (2.8 eq., 0.040 g, 0.108 mmol) was added and reaction mixture was stirred at room temperature for 30 minutes. After completion, reaction mixture was diluted with acetonitrile and purified by prep HPLC (38-47% acetonitrile in water with 0.1% TFA). Fractions containing the desired product were combined and lyophilized to dryness to afford Compound I-9 as a yellow solid. Yield: 0.0052 g, 15.6%; LC-MS m/z 846.1 [M+1]⁺; ¹H NMR (400 MHz, DMSO-d₆) δ8.65 (s, 1H), 7.92 (d, J=8.0 Hz, 1H), 7.83 (t, J=5.2 Hz, 1H), 7.71 (s, 1H), 7.64 (d, J=8.8 Hz, 2H), 7.20-6.94 (m, 2H), 6.62 (d, J=8.8 Hz, 2H), 4.49 (s, 2H), 4.45 (t, J=5.2 Hz, 2H), 4.34-4.29 (m, 1H), 3.81 (t, J=5.2 Hz, 2H), 3.74 (t, J=6.0 Hz, 2H), 3.05-2.99 (m, 4H), 2.58-2.55 (m, 2H), 2.23 (t, J=10.4 Hz, 2H), 1.96-1.93 (m, 1H), 1.86-1.80 (m, 1H), 1.55-1.49 (m, 2H), 1.43-1.38 (m, 2H).

Compound I-10 was prepared by reacting intermediate compound 3D with an appropriate azide containing intermediate by adapting the general method described above for compound I-10. The structure, name and data for compound I-10 is provided below:

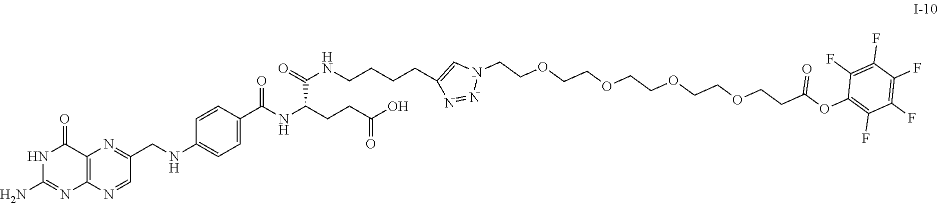 [SMILES: Nc1nc2ncc(CNc3ccc(C(=O)N[C@@H](CCC(=O)O)C(=O)NCCCCc4cn(CCOCCOCCOCCOCCC(=O)Oc5c(F)c(F)c(F)c(F)c5F)nn4)cc3)nc2c(=O)[nH]1]

### (S)-4-(4-(((2-amino-4-oxo-3,4-dihydropteridin-6-yl)methyl)amino)benzamido)-5-oxo-5-((4-(1-(15-oxo-15-(perfluorophenoxy)-3,6,9,12-tetraoxapentadecyl)-1H-1,2,3-triazol-4-yl)butyl)amino)pentanoic acid

LC-MS: m/z 978.30 [M+1]⁺.

¹H NMR (400 MHz, DMSO-d₆):″δ12.05 (bs, 1H), 11.41 (s, 1H), 8.64 (s, 1H), 7.92 (d, J=7.6 Hz, 1H), 7.83 (t, J=5.6 Hz, 1H), 7.76 (s, 1H), 7.64 (d, J=8.8 Hz, 2H), 6.94-6.83 (m, 2H), 6.62 (d, J=8.8 Hz, 2H), 4.48-4.47 (m, 2H), 4.43 (t, J=5.2 Hz, 2H), 4.34-4.29 (m, 1H), 3.77-3.73 (m, 4H), 3.55-3.44 (m, 13H), 3.06-2.99 (m, 4H), 2.58-2.56 (m, 2H), 2.23-2.22 (m, 2H), 1.96-1.93 (m, 1H), 1.86-1.80 (m, 1H), 1.57-1.53 (m, 2H), 1.43-1.39 (m, 2H).

### 6.1.5. Example 5: Compound I-11

### Synthesis of intermediate: (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol (Compound 121)

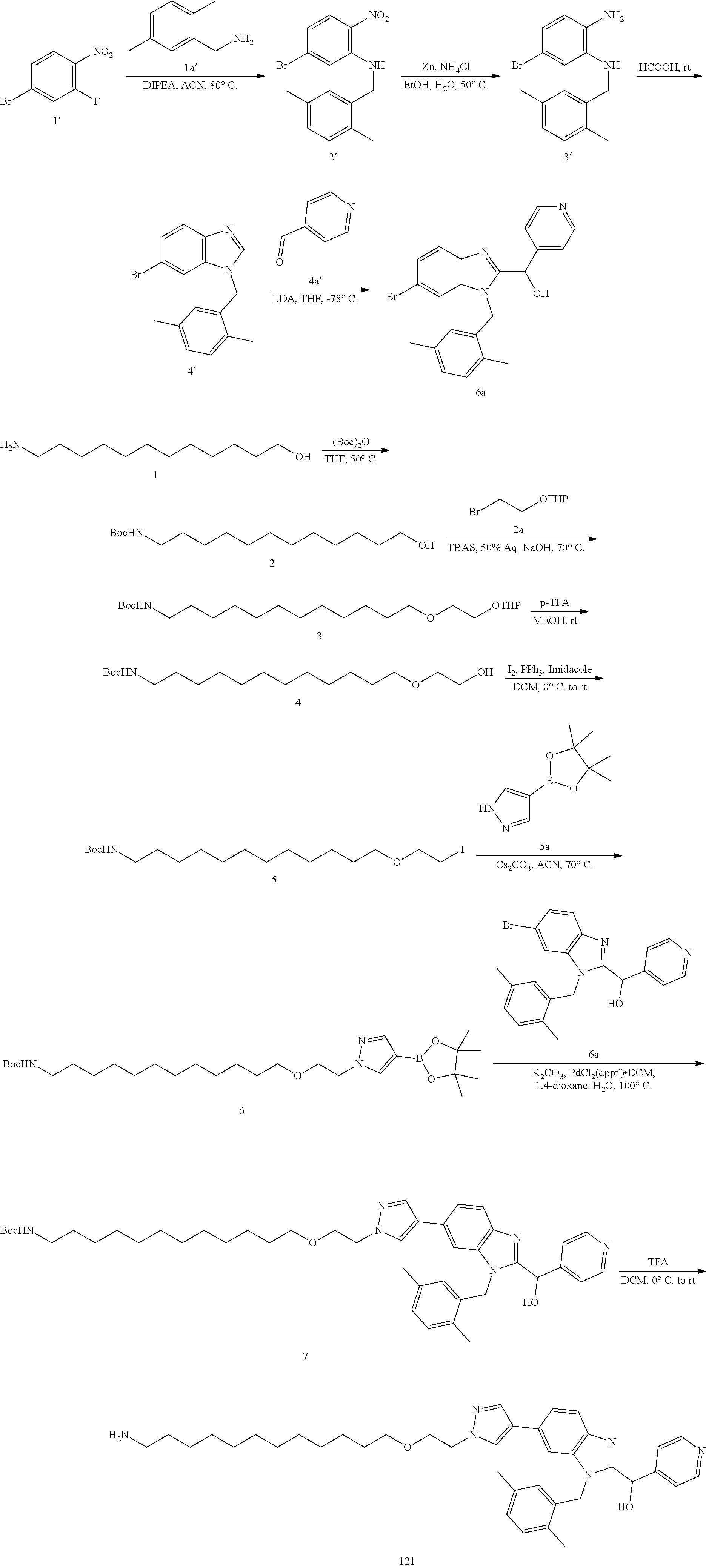

To a solution of 4-bromo-2-fluoro-1-nitrobenzene (1′, 1.0 eq, 10.0 g, 45.5 mmol) and (2,5-dimethylphenyl)methanamine (1a′, 1.1 eq, 6.76 g, 50.0 mmol) in acetonitrile (200 mL), N,N-diisopropylethylamine (3.0 eq, 24.5 mL, 136.0 mmol) was added and reaction was heated at 70° C. for 3 h. After completion, reaction mixture was diluted with water and extracted with ethyl acetate. The organic layer was dried over anhydrous sodium sulfate, filtered and concentrated to get 5-bromo-N-(2,5-dimethylbenzyl)-2-nitroaniline (2′) as a yellow solid. Yield: 15.0 g, 98.45%; LCMS m/z 334.96 [M+1]⁺.

To a solution of 5-bromo-N-(2,5-dimethylbenzyl)-2-nitroaniline (2′, 1.0 eq., 15.0 g, 44.7 mmol) in ethanol (360 mL) and water (40 mL), zinc powder (4.0 eq., 11.7 g, 179.0 mmol) and ammonium chloride (7.0 eq., 16.8 g, 313.0 mmol) were added and reaction mixture was heated at 50° C. for 3 h. After completion, reaction mixture was cooled, filtered through celite bed, celite bed was washed with ethyl acetate and filtrate was concentrated. Then, water was added and extracted with ethyl acetate. The organic layer was dried over anhydrous sodium sulfate, filtered and concentrated to get crude which was purified by column chromatography using silica gel (100-200 mesh) and 0-40% ethyl acetate in hexane to afford 5-bromo-N¹-(2,5-dimethylbenzyl)benzene-1,2-diamine (3′) as a brown solid. Yield: 12.0 g, 87.86%; LCMS m/z 305.08 [M+1].

5-bromo-N¹-(2,5-dimethylbenzyl)benzene-1,2-diamine (3′, 1.0 eq, 12.0 g, 39.3 mmol) and formic acid (120 mL) was stirred at room temperature for 16 h. After completion, reaction mixture was concentrated, ethyl acetate was added, washed with saturated aqueous sodium bicarbonate solution and water. The organic layer was dried over anhydrous sodium sulfate, filtered and concentrated to get crude which was purified by column chromatography using silica gel (100-200 mesh) and 0-50% ethyl acetate in hexane to afford 6-bromo-1-(2,5-dimethylbenzyl)-1H-benzo[d]imidazole (4′) as a pink solid. Yield: 8.7 g, 68.03%; LCMS m/z 315.03 [M+1]⁺.

A solution of 6-bromo-1-(2,5-dimethylbenzyl)-1H-benzo[d]imidazole (4′, 1.0 eq, 1.1 g, 3.49 mmol) in tetrahydrofuran (30 mL) was cooled at −78° C., lithium diisopropylamide (2.0 M in tetrahydrofuran, 2.5 eq, 4.36 mL, 8.72 mmol) was added and reaction mixture was stirred for 2 h at -78° C., then isonicotinaldehyde (4a′, 1.4 eq, 0.523 g, 4.89 mmol) was added and the reaction mixture was stirred at −78° C. for 30 minutes. After that, reaction mixture was quenched with saturated aqueous ammonium chloride solution and allowed to warm to room temperature. Then, reaction mixture was extracted with ethyl acetate. The organic layer was dried over anhydrous sodium sulfate, filtered and concentrated to get crude which was purified by column chromatography using silica gel (100-200 mesh) and 0-5% methanol in dichloromethane to afford (6-bromo-1-(2,5-dimethylbenzyl)-1H-benzo[d]imidazol-2-yl)(pyridin-4-yl)methanol (6a) as a brown solid. Yield: 1.0 g, 67.85%; LCMS m/z 422.08 [M+1]⁺.

A solution of 12-aminododecan-1-ol (1, 1.0 eq, 6.10 g, 30.3 mmol) and di-tert-butyl dicarbonate (1.1 eq, 8.08 mL, 33.3 mmol) in tetrahydrofuran (60 mL) was heated at 50° C. for 3 h. After completion, reaction mixture was concentrated to get crude which was purified by column chromatography using silica gel (100-200 mesh) and 0-25% ethyl acetate in hexane to afford tert-butyl (12-hydroxydodecyl)carbamate (2) as a white solid. Yield: 8.0 g, 87.59%; LCMS m/z 302.0 [M+1]⁺.

To a suspension of tert-butyl (12-hydroxydodecyl)carbamate (2, 1.0 eq, 5.0 g, 16.6 mmol) and 2-(2-bromoethoxy)tetrahydro-2H-pyran (2a, 2.0 eq, 6.94 g, 33.2 mmol) in 50% aqueous sodium hydroxide solution (50 mL), tetrabutylammonium sulfate (0.1 eq, 1.93 g, 1.66 mmol) was added and reaction mixture was heated at 70° C. for 16 h. After completion, reaction mixture was cooled, water was added and extracted with ethyl acetate. The organic layer was dried over anhydrous sodium sulfate, filtered and concentrated to get crude which was purified by column chromatography using silica gel (100-200 mesh) and 0-10% ethyl acetate in hexane to afford tert-butyl (12-(2-((tetrahydro-2H-pyran-2-yl)oxy)ethoxy)dodecyl)carbamate (3) as a colourless viscous liquid. Yield: 6.0 g, 82.83%; LCMS m/z 430.2 [M+1]⁺.

To a solution of tert-butyl (12-(2-((tetrahydro-2H-pyran-2-yl)oxy)ethoxy)dodecyl)carbamate (3, 1.0 eq, 6.0 g, 14.0 mmol) in methanol (60 mL), p-toluenesulfonic acid monohydrate (0.1 eq, 0.266 g, 1.40 mmol) was added and reaction mixture was stirred at room temperature for 4 h. After completion, reaction mixture was concentrated, diluted with ethyl acetate, washed with saturated aqueous sodium bicarbonate solution and water. The organic layer was dried over anhydrous sodium sulfate, filtered and concentrated to afford tert-butyl (12-(2-hydroxyethoxy)dodecyl)carbamate (4) as a colourless viscous liquid. Yield: 4.4 g, 91.19%; LCMS m/z 346.0 [M+1]⁺.

A solution of tert-butyl (12-(2-((tetrahydro-2H-pyran-2-yl)oxy)ethoxy)dodecyl)carbamate (4, 1.0 eq, 4.2 g, 12.2 mmol) in dichloromethane (40 mL) was cooled at 0° C., triphenylphosphine (1.5 eq, 4.78 g, 18.2 mmol), imidazole (1.5 eq, 1.24 g, 18.2 mmol) and iodine (1.5 eq, 4.63 g, 18.2 mmol) were added and reaction mixture was stirred at room temperature for 2 h. After that, reaction mixture was diluted with water and extracted with dichloromethane. The organic layer was dried over sodium sulfate, filtered and concentrated to get crude which was purified by column chromatography using silica gel (100-200 mesh) and 0-7% ethyl acetate in hexane to afford tert-butyl (12-(2-iodoethoxy)dodecyl)carbamate (5) as an off white solid. Yield: 4.0 g, 72.26%; LCMS m/z 456.0 [M+1]⁺.

A suspension of 4-(4,4,5,5-tetramethyl-1,3,2-dioxaborolan-2-yl)-1H-pyrazole (5a, 1.0 eq, 1.0 g, 5.15 mmol), tert-butyl (12-(2-iodoethoxy)dodecyl)carbamate (5, 0.8 eq, 1.88 g, 4.12 mmol) and cesium carbonate (2.0 eq, 3.36 g, 10.3 mmol) in acetonitrile (20 mL) was heated at 70° C. for 16 h. After completion, reaction mixture was cooled and concentrated to get crude which was purified by column chromatography using silica gel (100-200 mesh) and 0-30% ethyl acetate in hexane to afford tert-butyl (12-(2-(4-(4,4,5,5-tetramethyl-1,3,2-dioxaborolan-2-yl)-1H-pyrazol-1-yl)ethoxy)dodecyl)carbamate (6) as a colourless viscous liquid. Yield: 1.9 g, 70.37%; LCMS m/z 522.2 [M+1]⁺.

A solution of (6-bromo-1-(2,5-dimethylbenzyl)-1H-benzo[d]imidazol-2-yl)(pyridin-4-yl)methanol (6a, 1.0 eq, 1.0 g, 2.37 mmol), tert-butyl (12-(2-(4-(4,4,5,5-tetramethyl-1,3,2-dioxaborolan-2-yl)-1H-pyrazol-1-yl)ethoxy)dodecyl)carbamate (6, 1.5 eq, 1.85 g, 3.55 mmol) and potassium carbonate (2.0 eq, 0.654 g, 4.74 mmol) in 1,4-dioxane (8 mL) and water (2 mL) was degassed under nitrogen for 5 minutes. Then, [1,1′-bis(diphenylphosphino)ferrocene]dichloropalladium(II) complex with dichloromethane (0.05 eq, 0.096 g, 0.118 mmol) was added and reaction mixture was heated at 100° C. for 16 h. After completion, reaction mixture was cooled, water was added and extracted with ethyl acetate. The organic layer was dried over anhydrous sodium sulfate, filtered and concentrated to get crude which was purified by column chromatography using silica gel (100-200 mesh) and 0-8% methanol in dichloromethane to afford tert-butyl (12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)carbamate (7) as a brown solid. Yield: 1.25 g, 71.63%; LCMS m/z 737.36 [M+1]⁺.

A solution of tert-butyl (12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)carbamate (7, 1.0 eq, 1.25 g, 1.70 mmol) in dichloromethane (6 mL) was cooled at 0° C., trifluoroacetic acid (6 mL) was added dropwise and reaction mixture was stirred at room temperature for 3 h. After completion, reaction mixture was concentrated, water was added, neutralized with solid sodium bicarbonate and extracted with 10% methanol in dichloromethane. The organic layer was dried over anhydrous sodium sulfate, filtered and concentrated to get crude which was purified by column chromatography using silica gel (100-200 mesh) and 0-20% methanol in dichloromethane to afford (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol (Compound 121) as a light brown solid. Yield: 0.800 g, 73.11%; LCMS m/z 637.34 [M+1]+; ¹H NMR (400 MHz, DMSO-d₆) δ8.38 (d, J=6.0 Hz, 2H), 8.03 (s, 1H), 7.78 (s, 1H), 7.62 (d, J=8.4 Hz, 1H), 7.44-7.41 (m, 2H), 7.29-7.26 (m, 2H), 7.05 (d, J=7.6 Hz, 1H), 6.86 (d, J=7.6 Hz, 1H), 6.01 (s, 1H), 5.81 (s, 1H), 5.59 (d, J=17.6 Hz, 1H), 5.45 (d, J=17.2 Hz, 1H), 4.20 (t, J=5.2 Hz, 2H), 3.69 (t, J=5.2 Hz, 2H), 2.58-2.54 (m, 2H), 2.32 (s, 3H), 1.90 (s, 3H), 1.38-1.33 (m, 4H), 1.23-1.13 (m, 20H).

### Synthesis of N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutamine (Compound I-11)

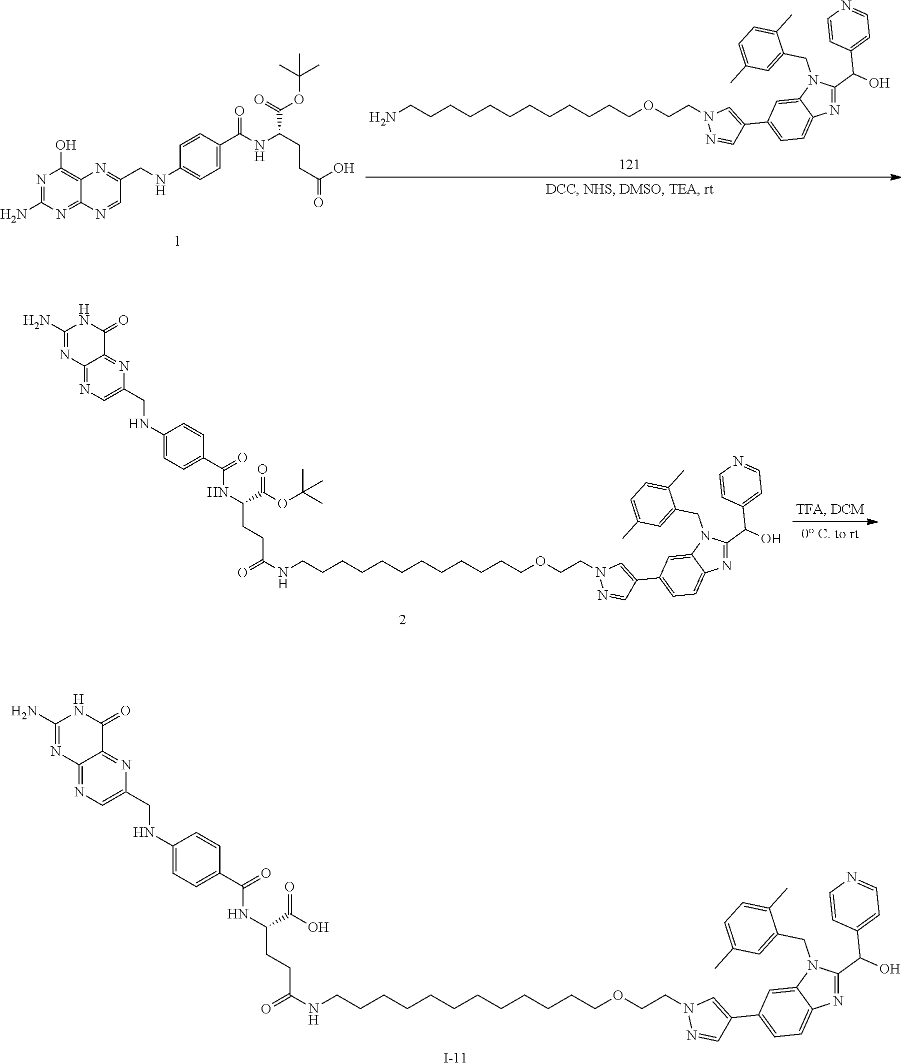

To a solution of 4-bromo-2-fluoro-1-nitrobenzene (1.0 eq, 10.0 g, 45.5 mmol) and (2,5-dimethylphenyl)methanamine (1.1 eq, 6.76 g, 50.0 mmol) in acetonitrile (200 mL), N,N-diisopropylethylamine (3.0 eq, 24.5 mL, 136.0 mmol) was added and reaction was heated at 70° C. for 3 h. After completion, reaction mixture was diluted with water and extracted with ethyl acetate. The organic layer was dried over anhydrous sodium sulfate, filtered and concentrated to get 5-bromo-N-(2,5-dimethylbenzyl)-2-nitroaniline as a yellow solid. Yield: 15.0 g, 98.4%; LCMS m/z 334.9 [M+1]⁺.

To a solution of 5-bromo-N-(2,5-dimethylbenzyl)-2-nitroaniline (1.0 eq., 15.0 g, 44.7 mmol) in ethanol (360 mL) and water (40 mL), zinc powder (4.0 eq., 11.7 g, 179.0 mmol) and ammonium chloride (7.0 eq., 16.8 g, 313.0 mmol) were added and reaction mixture was heated at 50° C. for 3 h. After completion, reaction mixture was cooled, filtered through celite bed, celite bed was washed with ethyl acetate and filtrate was concentrated. Then, water was added and the mixture extracted with ethyl acetate. The organic layer was dried over anhydrous sodium sulfate, filtered and concentrated to get crude which was purified by column chromatography using silica gel (100-200 mesh) and 0-40% ethyl acetate in hexane to afford 5-bromo-N¹-(2,5-dimethylbenzyl)benzene-1,2-diamine as a brown solid. Yield: 12.0 g, 87.8%; LCMS m/z 305.1 [M+1]⁺.

5-bromo-N¹-(2,5-dimethylbenzyl)benzene-1,2-diamine (1.0 eq, 12.0 g, 39.3 mmol) and formic acid (120 mL) was stirred at room temperature for 16 h. After completion, reaction mixture was concentrated, ethyl acetate was added, washed with saturated aqueous sodium bicarbonate solution and water. The organic layer was dried over anhydrous sodium sulfate, filtered and concentrated to get crude which was purified by column chromatography using silica gel (100-200 mesh) and 0-50% ethyl acetate in hexane to afford 6-bromo-1-(2,5-dimethylbenzyl)-1H-benzo[d]imidazole as a pink solid. Yield: 8.7 g, 68.0%; LCMS m/z 315.0 [M+1]⁺.

A solution of 6-bromo-1-(2,5-dimethylbenzyl)-1H-benzo[d]imidazole (1.0 eq, 1.1 g, 3.49 mmol) in tetrahydrofuran (30 mL) was cooled at −78° C., lithium diisopropylamide (2.0 M in tetrahydrofuran, 2.5 eq, 4.36 mL, 8.72 mmol) was added and reaction mixture was stirred for 2 h at -78° C., then isonicotinaldehyde (4a′, 1.4 eq, 0.523 g, 4.89 mmol) was added and the reaction mixture was stirred at −78° C. for 30 minutes. After that, the reaction mixture was quenched with saturated aqueous ammonium chloride solution and allowed to warm to room temperature. Then, the reaction mixture was extracted with ethyl acetate. The organic layer was dried over anhydrous sodium sulfate, filtered and concentrated to get crude product which was purified by column chromatography using silica gel (100-200 mesh) and 0-5% methanol in dichloromethane to afford (6-bromo-1-(2,5-dimethylbenzyl)-1H-benzo[d]imidazol-2-yl)(pyridin-4-yl)methanol as a brown solid. Yield: 1.0 g, 67.8%; LCMS m/z 422.0 [M+1]⁺.

A solution of 12-aminododecan-1-ol (1.0 eq, 6.10 g, 30.3 mmol) and di-tert-butyl dicarbonate (1.1 eq, 8.08 mL, 33.3 mmol) in tetrahydrofuran (60 mL) was heated at 50° C. for 3 h. After completion, the reaction mixture was concentrated to get crude material which was purified by column chromatography using silica gel (100-200 mesh) and 0-25% ethyl acetate in hexane to afford tert-butyl (12-hydroxydodecyl)carbamate as awhite solid. Yield: 8.0 g, 87.6%; LCMS m/z 302.0 [M+1]⁺.

To a suspension of tert-butyl (12-hydroxydodecyl)carbamate (1.0 eq, 5.0 g, 16.6 mmol) and 2-(2-bromoethoxy)tetrahydro-2H-pyran (2.0 eq, 6.94 g, 33.2 mmol) in 50% aqueous sodium hydroxide solution (50 mL), tetrabutylammonium sulfate (0.1 eq, 1.93 g, 1.66 mmol) was added and reaction mixture was heated at 70° C. for 16 h. After completion, reaction mixture was cooled, water was added and extracted with ethyl acetate. The organic layer was dried over anhydrous sodium sulfate, filtered and concentrated to get crude material which was purified by column chromatography using silica gel (100-200 mesh) and 0-10% ethyl acetate in hexane to afford tert-butyl (12-(2-((tetrahydro-2H-pyran-2-yl)oxy)ethoxy)dodecyl)carbamate as a colorless viscous liquid. Yield: 6.0 g, 82.8%; LCMS m/z 430.2 [M+1]⁺.

To a solution of tert-butyl (12-(2-((tetrahydro-2H-pyran-2-yl)oxy)ethoxy)dodecyl)carbamate (1.0 eq, 6.0 g, 14.0 mmol) in methanol (60 mL), p-toluenesulfonic acid monohydrate (0.1 eq, 0.266 g, 1.40 mmol) was added and reaction mixture was stirred at room temperature for 4 h. After completion, reaction mixture was concentrated, diluted with ethyl acetate, washed with saturated aqueous sodium bicarbonate solution and water. The organic layer was dried over anhydrous sodium sulfate, filtered and concentrated to afford tert-butyl (12-(2-hydroxyethoxy)dodecyl)carbamate as a colorless viscous liquid. Yield: 4.4 g, 91.1%; LCMS m/z 346.0 [M+1]⁺.

A solution of tert-butyl (12-(2-((tetrahydro-2H-pyran-2-yl)oxy)ethoxy)dodecyl)carbamate (1.0 eq, 4.2 g, 12.2 mmol) in dichloromethane (40 mL) was cooled at 0° C., triphenylphosphine (1.5 eq, 4.78 g, 18.2 mmol), imidazole (1.5 eq, 1.24 g, 18.2 mmol) and iodine (1.5 eq, 4.63 g, 18.2 mmol) were added and reaction mixture was stirred at room temperature for 2 h. After that, reaction mixture was diluted with water and extracted with dichloromethane. The organic layer was dried over sodium sulfate, filtered and concentrated to get crude which was purified by column chromatography using silica gel (100-200 mesh) and 0-7% ethyl acetate in hexane to afford tert-butyl (12-(2-iodoethoxy)dodecyl)carbamate as an off white solid. Yield: 4.0 g, 72.2%; LCMS m/z 456.0 [M+1]⁺.

A suspension of 4-(4,4,5,5-tetramethyl-1,3,2-dioxaborolan-2-yl)-1H-pyrazole (1.0 eq, 1.0 g, 5.15 mmol), tert-butyl (12-(2-iodoethoxy)dodecyl)carbamate (0.8 eq, 1.88 g, 4.12 mmol) and cesium carbonate (2.0 eq, 3.36 g, 10.3 mmol) in acetonitrile (20 mL) was heated at 70° C. for 16 h. After completion, reaction mixture was cooled and concentrated to get crude which was purified by column chromatography using silica gel (100-200 mesh) and 0-30% ethyl acetate in hexane to afford tert-butyl (12-(2-(4-(4,4,5,5-tetramethyl-1,3,2-dioxaborolan-2-yl)-1H-pyrazol-1-yl)ethoxy)dodecyl)carbamate as a colorless viscous liquid. Yield: 1.9 g, 70.3%; LCMS m/z 522.2 [M+1]⁺.

A solution of (6-bromo-1-(2,5-dimethylbenzyl)-1H-benzo[d]imidazol-2-yl)(pyridin-4-yl)methanol (1.0 eq, 1.0 g, 2.37 mmol), tert-butyl (12-(2-(4-(4,4,5,5-tetramethyl-1,3,2-dioxaborolan-2-yl)-1H-pyrazol-1-yl)ethoxy)dodecyl)carbamate (1.5 eq, 1.85 g, 3.55 mmol) and potassium carbonate (2.0 eq, 0.654 g, 4.74 mmol) in 1,4-dioxane (8 mL) and water (2 mL) was degassed under nitrogen for 5 minutes. Then, [1,1′-bis(diphenylphosphino)ferrocene]dichloropalladium(II) complex with dichloromethane (0.05 eq, 0.096 g, 0.118 mmol) was added and reaction mixture was heated at 100° C. for 16 h. After completion, reaction mixture was cooled, water was added and extracted with ethyl acetate. The organic layer was dried over anhydrous sodium sulfate, filtered and concentrated to get crude which was purified by column chromatography using silica gel (100-200 mesh) and 0-8% methanol in dichloromethane to afford tert-butyl (12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)carbamate as a brown solid. Yield: 1.25 g, 71.6%; LCMS m/z 737.3 [M+1]⁺.

A solution of tert-butyl (12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)carbamate (1.0 eq, 1.25 g, 1.70 mmol) in dichloromethane (6 mL) was cooled at 0° C., trifluoroacetic acid (6 mL) was added dropwise and reaction mixture was stirred at room temperature for 3 h. After completion, reaction mixture was concentrated, water was added, neutralized with solid sodium bicarbonate and extracted with 10% methanol in dichloromethane. The organic layer was dried over anhydrous sodium sulfate, filtered and concentrated to get crude which was purified by column chromatography using silica gel (100-200 mesh) and 0-20% methanol in dichloromethane to afford (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol as a light brown solid. Yield: 0.800 g, 73.1%; LCMS m/z 637.3 [M+1]⁺; H NMR (400 MHz, DMSO-d₆) δ8.38 (d, J=6.0 Hz, 2H), 8.03 (s, 1H), 7.78 (s, 1H), 7.62 (d, J=8.4 Hz, 1H), 7.44-7.41 (m, 2H), 7.29-7.26 (m, 2H), 7.05 (d, J=7.6 Hz, 1H), 6.86 (d, J=7.6 Hz, 1H), 6.01 (s, 1H), 5.81 (s, 1H), 5.59 (d, J=17.6 Hz, 1H), 5.45 (d, J=17.2 Hz, 1H), 4.20 (t, J=5.2 Hz, 2H), 3.69 (t, J=5.2 Hz, 2H), 2.58-2.54 (m, 2H), 2.32 (s, 3H), 1.90 (s, 3H), 1.38-1.33 (m, 4H), 1.23-1.13 (m, 20H).

To a solution of (S)-4-(4-(((2-amino-4-hydroxypteridin-6-yl)methyl)amino)benzamido)-5-(tert-butoxy)-5-oxopentanoic acid (1, 1.0 eq, 0.200 g, 0.402 mmol) in dimethylsulfoxide (20 mL), 1-hydroxypyrrolidine-2,5-dione (2.0 eq, 0.0925 g, 0.804 mmol) and N,N′-dicyclohexylmethanediimine (2.0 eq, 0.166 g, 0.804 mmol) were added under nitrogen atmosphere in dark (covered with aluminum foil). The reaction mixture was stirred at room temperature for 16 h. After the completion of reaction, the reaction mixture was filtered through sintered glass filter. To the filtrate, a solution of (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol (1.0 eq, 0.25 g, 0.402 mmol) in dimethylsulfoxide (2 mL) followed by triethylamine (2.0 eq, 0.11 mL, 0.081 mmol) were added. The reaction mixture was stirred under dark condition (covered with Aluminum foil) at 35° C. for 16 h. After the completion of reaction, reaction mixture was purified by reverse phase column chromatography (using 40 g C-18 spherical column 30-40% acetonitrile in water). The desired fractions were concentrated under reduced pressure to afford tert-butyl N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutaminate (2) as a yellow solid. Yield 0.08 g, 17%; LCMS m/z 1116.7 [M+1]⁺.

To a stirred solution of tert-butyl N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutaminate (2, 1.0 eq, 0.08 g, 0.071 mmol) in dichloromethane (2.5 mL) at 0° C., trifluoroacetic acid (0.5 mL) was added and reaction mixture was stirred at room temperature for 3 h. After completion, reaction mixture was concentrated to get crude. The crude was purified by prep-HPLC using (30-40% acetonitrile in water with 0.1% trifluoroacetic acid). Fractions containing the desired product were combined and lyophilized to dryness to afford N2-(4-(((2-amino-4-oxo-3,4-dihydropteridin-6-yl)methyl)amino)benzoyl)-N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-L-glutamine (Cpd. No. I-11) as a yellow solid. Yield 0.003 g, 4%; LCMS m/z 531.2 [M+2]++; ¹H NMR (400 MHz, DMSO-d6) δ8.64 (s, 1H), 8.55 (d, J=5.2 Hz, 2H), 8.19 (d, J=14.8 Hz, 1H), 8.07 (s, 1H), 7.82-7.79 (m, 2H), 7.66-7.63 (m, 3H), 7.59-7.57 (m, 3H), 7.49 (d, J=8.4 Hz, 1H), 7.07-7.04 (m, 3H), 6.86 (d, J=7.2 Hz, 1H), 6.63 (d, J=8.4 Hz, 2H), 6.23 (s, 1H), 5.76 (s, 1H), 5.64 (d, J=16.8 Hz, 1H), 5.52 (d, J=17.2 Hz, 2H), 4.48 (s, 2H), 4.27-4.19 (m, 3H), 3.70-3.69 (m, 3H), 3.31 (t, J=6.0 Hz, 3H), 3.01-2.95 (m, 2H), 2.33 (s, 3H), 2.17-2.15 (m, 2H), 2.04-2.02 (m, 1H), 1.89 (s, 4H), 1.38-1.32 (m, 4H), 1.15-1.11 (m, 17H).

### 6.1.6. Example 6: Compound I-12

### N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-N2-(5-(methyl((2-methyl-4-oxo-1,4-dihydroquinazolin-6-yl)methyl)amino)thiophene-2-carbonyl)-L-glutamine (Compound I-12)

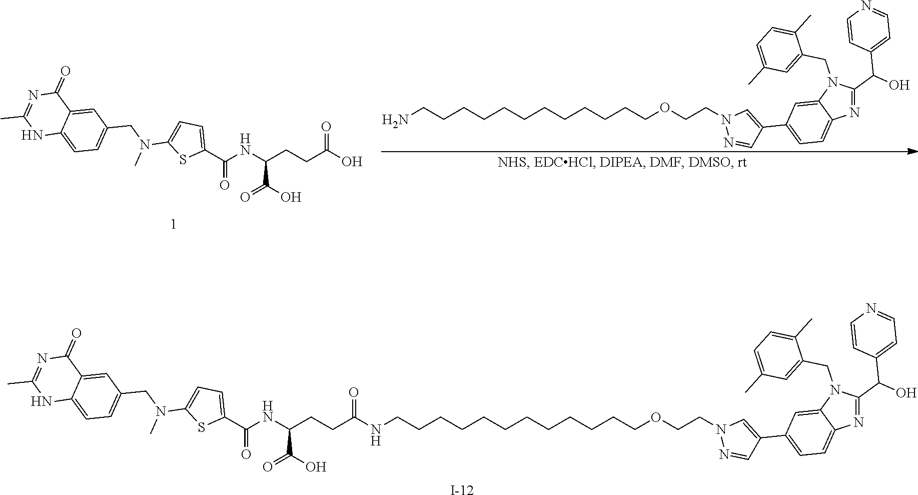

To a solution of (5-(methyl((2-methyl-4-oxo-1,4-dihydroquinazolin-6-yl)methyl)amino)thiophene-2-carbonyl)-L-glutamic acid (1, 1.0 eq, 0.070 g, 0.153 mmol) in N,N-dimethylformamide (1 mL) and dimethyl sulfoxide (1 mL), N-hydroxysuccinimide (1.1 eq, 0.019 g, 0.168 mmol), 1-(3-Dimethylaminopropyl)-3-ethylcarbodiimide hydrochloride (EDC·HCl) (1.1 eq, 0.032 g, 0.168 mmol) and N,N-diisopropylethylamine (3.0 eq, 0.084 mL, 0.458 mmol) were added and reaction mixture was stirred at room temperature for 30 minutes. Then, (6-(1-(2-((12-aminododecyl)oxy)ethyl)-1H-pyrazol-4-yl)-1-(2,5-dimethylbenzyl)-1H-benzo[d]imidazol-2-yl)(pyridin-4-yl)methanol (1.0 eq, 0.097 g, 0.153 mmol) was added and reaction mixture was stirred at room temperature for 16 h. After completion, reaction mixture was directly purified by prep HPLC (30-45% acetonitrile in water with 0.1% TFA). Fractions containing the desired product were combined and lyophilized to dryness to afford N5-(12-(2-(4-(1-(2,5-dimethylbenzyl)-2-(hydroxy(pyridin-4-yl)methyl)-1H-benzo[d]imidazol-6-yl)-1H-pyrazol-1-yl)ethoxy)dodecyl)-N2-(5-(methyl((2-methyl-4-oxo-1,4-dihydroquinazolin-6-yl)methyl)amino)thiophene-2-carbonyl)-L-glutamine (Cpd. No. I-12) as a yellow solid. Yield: 0.028 g, 17.0%; LC-MS m/z 539.5 [M+2]++; ¹H NMR (400 MHz, DMSO-d6 with D₂O) δ8.24 (d, J=5.6 Hz, 2H), 7.90 (s, 1H), 7.85 (s, 1H), 7.72 (s, 1H), 7.61 (d, J=8.4 Hz, 2H), 7.49 (d, J=8.4 Hz, 1H), 7.41-7.39 (m, 2H), 7.28 (s, 1H), 7.22 (d, J=5.6 Hz, 2H), 6.96 (d, J=7.6 Hz, 1H), 6.77 (d, J=7.6 Hz, 1H), 6.04 (s, 1H), 5.87-5.84 (m, 1H), 5.59 (s, 1H), 5.51 (d, J=16.8 Hz, 1H), 5.34 (d, J=16.8 Hz, 1H), 4.53 (s, 2H), 3.63-3.62 (m, 2H), 3.20 (t, J=6.0 Hz, 2H), 2.96 (s, 3H), 2.91 (t, J=6.8 Hz, 2H), 2.29 (s, 3H), 2.21 (s, 3H), 2.12-2.10 (m, 2H), 1.97-1.95 (m, 1H), 1.89 (d, J=4.0 Hz, 1H), 1.86-1.73 (m, 1H), 1.77 (d, J=8.0 Hz, 2H), 1.27-1.21 (m, 5H), 1.04-0.83 (m, 15H).

### 6.1.7. Example 7: Compound I-13

### N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutamine (Compound I-13)

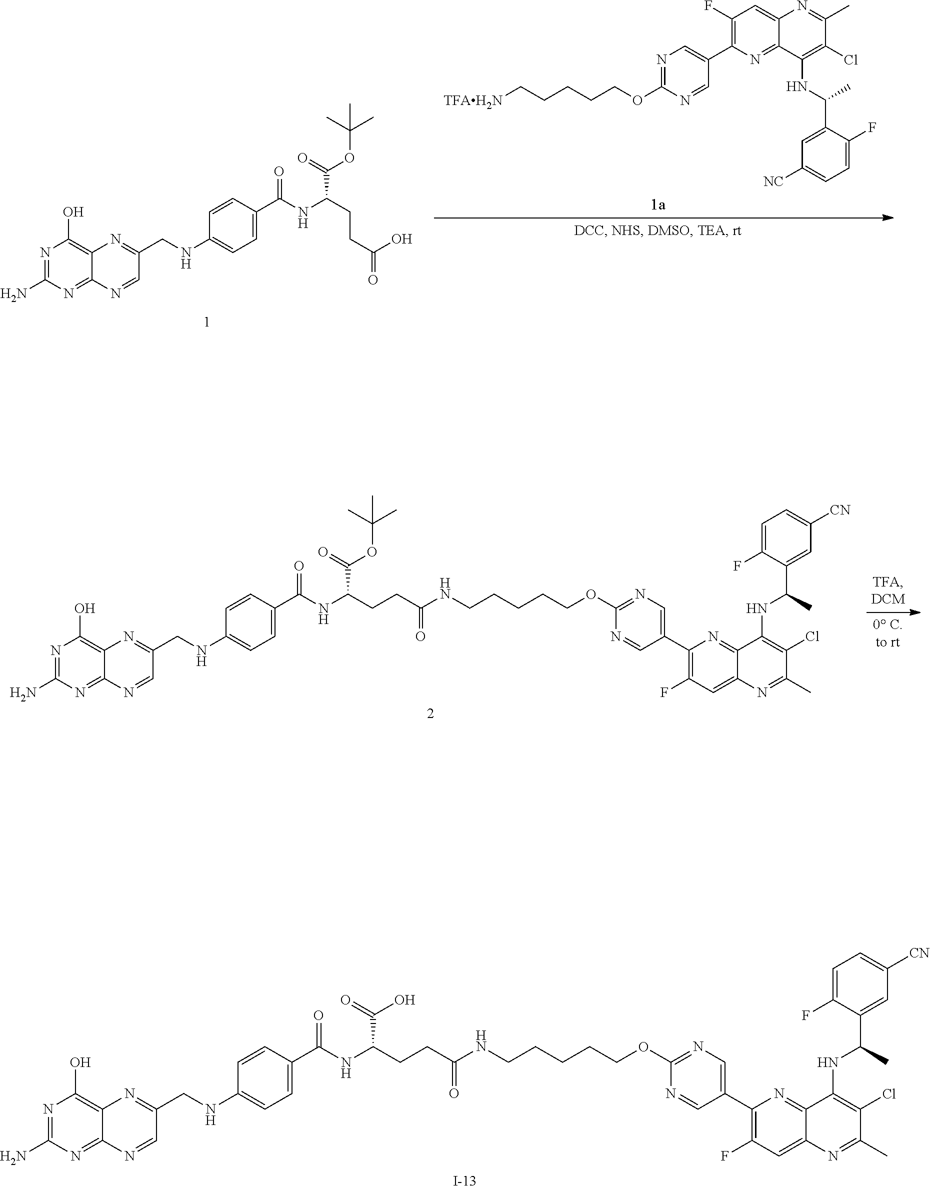

To a solution of (S)-4-(4-(((2-amino-4-hydroxypteridin-6-yl)methyl)amino)benzamido)-5-(tert-butoxy)-5-oxopentanoic acid (1, 1.0 eq, 0.200 g, 0.402 mmol) in dimethylsulfoxide (20 mL), 1-hydroxypyrrolidine-2,5-dione (2.0 eq, 0.0925 g, 0.804 mmol), and N,N′-dicyclohexylmethanediimine (2.0 eq, 0.166 g, 0.804 mmol) were added under nitrogen atmosphere in dark (covered with Aluminum foil). The reaction mixture was stirred at room temperature for 16 h. After completion (monitored by LCMS), the reaction mixture was filtered through sintered. To the filtrate, a solution of (R)-3-(1-((3-chloro-7-fluoro-2-methyl-6-(2-((5-((2,2,2-trifluoroacetyl)-14-azaneyl)pentyl)oxy)pyrimidin-5-yl)-1,5-naphthyridin-4-yl)amino)ethyl)-4-fluorobenzonitrile (TFA salt) (1a, 1.0 eq, 0.25 g, 0.402 mmol) in dimethylsulfoxide (2 mL) followed by triethylamine (4.0 eq, 0.22 mL, 1.61 mmol) were added. The reaction mixture was stirred under dark condition (covered with Aluminium foil) at 35° C. for 16 h. After completion, the reaction mixture was purified by reverse phase column chromatography (using 40 g C-18 spherical column 30-60% acetonitrile in water). The desired fractions were concentrated under reduced pressure to afford tert-butyl N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutaminate (2) as a yellow solid. Yield: 0.10 g, 24%; LCMS: m/z 1018.5 [M+1]⁺.

To a stirred solution of tert-butyl N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutaminate (2, 1.0 eq, 0.10 g, 0.098 mmol) in dichloromethane (5 mL) at 0° C., trifluoroacetic acid (1.0 mL) was added and reaction mixture was stirred at room temperature for 3 h. After completion, the reaction mixture was concentrated to get crude product. The crude material was purified by prep-HPLC using (eluting from a C₁₈ column, with 20-60% acetonitrile in water with 0.1% trifluoroacetic acid). Fractions containing desired compound were lyophilized to dryness to afford N2-(4-(((2-amino-4-hydroxypteridin-6-yl)methyl)amino)benzoyl)-N5-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-L-glutamine (Cpd. No. I-13) as a yellow solid. Yield: 0.025 g, 26.4%; LCMS: m/z 481.4 [M+2]++; ¹H NMR (400 MHz, DMSO-d6 with D₂O) δ8.89 (s, 2H), 8.61 (s, 1H), 8.06 (d, J=10.8 Hz, 1H), 7.90 (d, J=6.0 Hz, 1H), 7.75-7.70 (m, 1H), 7.59 (d, J=8.8 Hz, 2H), 7.24 (t, J=10.0 Hz, 1H), 6.60 (d, J=8.4 Hz, 2H), 6.49-6.46 (m, 1H), 4.44 (s, 2H), 4.34 (t, J=6.4 Hz, 2H), 4.25-4.22 (m, 1H), 3.03-3.01 (m, 2H), 2.67 (s, 3H), 2.19-2.16 (m, 2H), 2.05-2.01 (m, 1H), 1.92-1.90 (m, 1H), 1.73-1.69 (m, 2H), 1.66 (d, J=6.8 Hz, 3H), 1.41-1.36 (m, 4H).

### 6.1.8. Example 8: Compound I-14

### N2-(4-(((2-amino-4-hydroxypteridin-6-yl)methyl)amino)benzoyl)-N5-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methyl)-L-glutamine (Compound I-14)

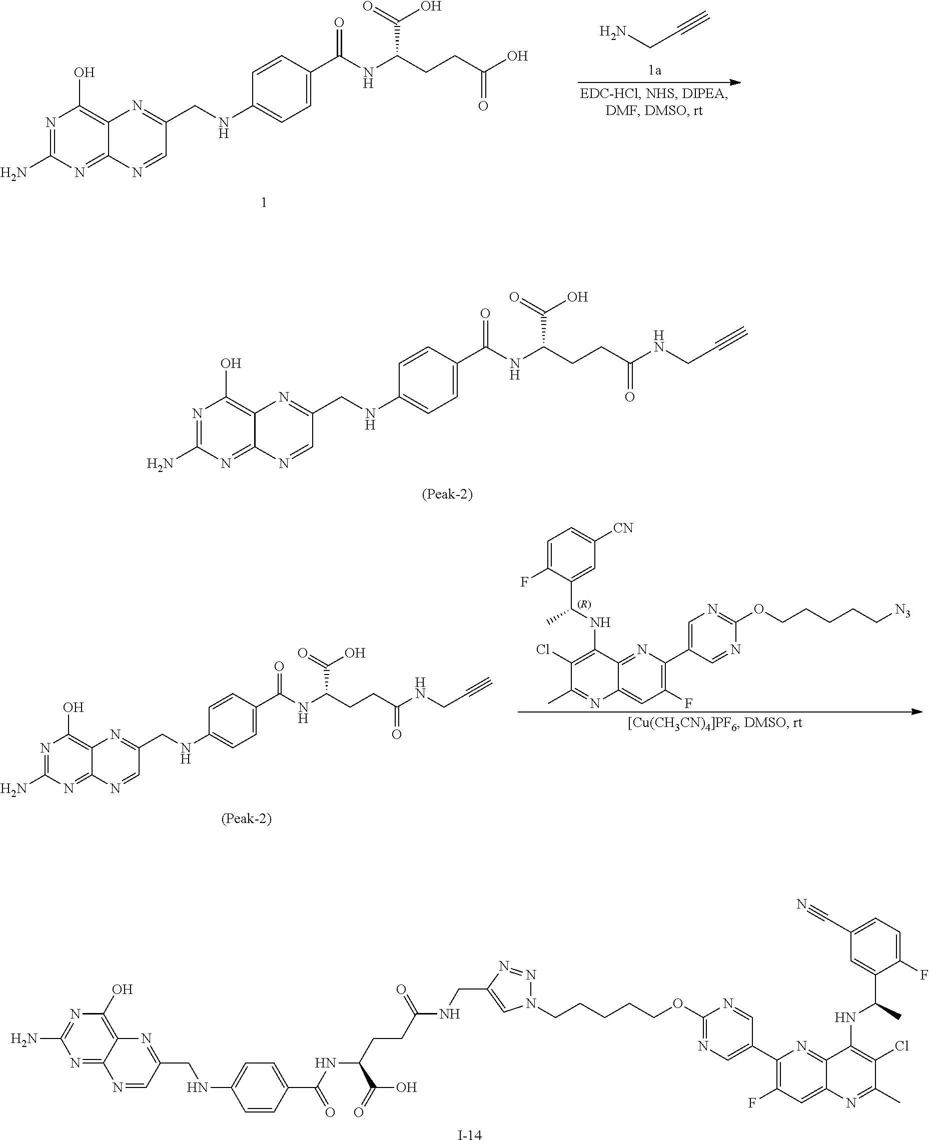

To a suspension of Folic acid (1, 1.0 eq, 2.0 g, 4.53 mmol) in N,N-dimethylformamide (40 mL) and dimethyl sulfoxide (40 mL), N-hydroxysuccinimide (1.5 eq, 0.782 g, 6.80 mmol), 1-(3-Dimethylaminopropyl)-3-ethylcarbodiimide hydrochloride (EDC·HCl) (1.5 eq, 1.3 g, 6.80 mmol) and N,N-diisopropylethylamine (3.0 eq, 2.51 mL, 13.6 mmol) were added and reaction mixture was stirred at room temperature for 30 minutes. Then, prop-2-yn-1-amine (1a, 1.5 eq, 0.374 g, 6.80 mmol) was added and reaction mixture was stirred at room temperature for 16 h. After completion, reaction mixture was directly purified by prep HPLC (10-25% acetonitrile in water with 0.1% acetic acid). Fractions containing the desired product were combined and lyophilized to dryness to afford N2-(4-(((2-amino-4-hydroxypteridin-6-yl)methyl)amino)benzoyl)-N5-(prop-2-yn-1-yl)-L-glutamine (Peak-2) as a yellow solid. Yield: 0.250 g, 11.5%; LCMS m/z 479.3 [M+1]⁺.

To a solution of N2-(4-(((2-amino-4-hydroxypteridin-6-yl)methyl)amino)benzoyl)-N5-(prop-2-yn-1-yl)-L-glutamine (Peak-2, 1.0 eq, 0.030 g, 0.062 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.01 eq, 0.035 g, 0.063 mmol) in dimethylsulfoxide (1.5 mL), tetrakis(acetonitrile)copper(I) hexafluorophosphate (2.8 eq, 0.065 g, 0.176 mmol) was added and reaction mixture was stirred at room temperature for 1 h. After completion, reaction mixture was directly purified by prep HPLC (20-48% acetonitrile in water with 0.1% formic acid). Fractions containing the desired product were combined and lyophilized to dryness to afford N2-(4-(((2-amino-4-hydroxypteridin-6-yl)methyl)amino)benzoyl)-N5-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methyl)-L-glutamine (Cpd. No. I-14) as a yellow solid. Yield: 0.015 g, 22.9%; LCMS m/z 1042.3 [M+1]⁺; ¹H-NMR (400 MHz, DMSO-d6 with D₂O) δ9.00 (s, 2H), 8.61 (s, 1H), 8.11 (d, J=11.6 Hz, 1H), 7.96 (dd, J=1.6 Hz, 6.8 Hz, 1H), 7.86 (s, 1H), 7.76-7.73 (m, 1H), 7.62 (d, J=8.8 Hz, 2H), 7.27 (d, J=10.0 Hz, 1H), 6.62 (d, J=8.8 Hz, 2H), 6.35 (d, J=7.6 Hz, 1H), 4.45 (s, 2H), 4.38 (t, J=6.4 Hz, 2H), 4.33-4.20 (m, 5H), 2.63 (s, 3H), 2.50-2.49 (m, 1H), 2.24-2.20 (m, 2H), 2.08-2.06 (m, 1H), 1.92-1.75 (m, 5H), 1.64 (d, J=6.8 Hz, 3H), 1.39-1.35 (m, 2H).

Compounds I-15 to I-18 were also prepared from compound 1 by adapting the general method described above for compound I-14. Table X outlines the data for each of the compounds that were synthesized according to the general method outlined for the synthesis of compound I-3.

TABLE X
Data for compounds synthesized from compound 1

| # | Compound structure, name and Data |
|---|---|
| I-15 | 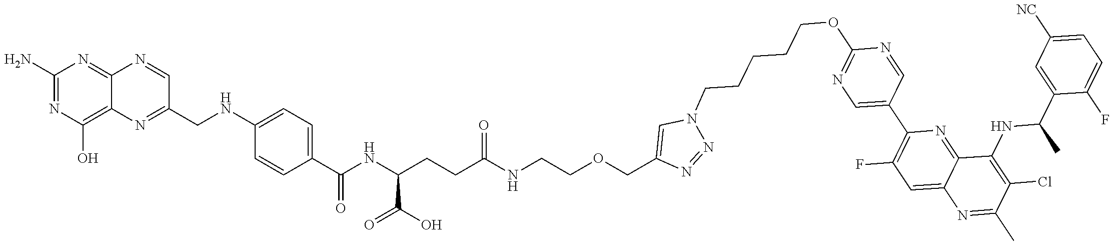 [SMILES: Cc1nc2cc(F)c(-c3cnc(OCCCCCn4cc(COCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| | N2-(4-(((2-amino-4-hydroxypteridin-6-yl)methyl)amino)benzoyl)-N5-(2-((1-(5-((5-(7- |
| | chloro-8-((I-1-(5-cyano-2-fluorophenyl)ethyl)amino)-3-fluoro-6-methyl-1,5-naphthyridin- |
| | 2-yl)pyrimidin-2-yl)oxy)pentyl)-1H-1,2,3-triazol 4-yl)methoxy)ethyl)-L-glutamine |
| | LCMS m/z 1086.3 [M + 1]⁺ |
| | ¹H NMR (400 MHz, DMSO-d6 with D₂O) δ 8.88 (s, 2H), 8.61 (s, 1H), 8.05 (d, J = 10.4 Hz, |
| | 1H), 8.00 (s, 1H), 7.89-7.88 (s, 1H), 7.72 (bs, 1H), 7.58 (d, J = 8.4 Hz, 2H), 7.22 (t, J = 9.6 |
| | Hz, 1H), 6.59 (d, J = 6.0 Hz, 2H), 6.49-6.48 (m, 1H), 4.44 (s, 4H), 4.37-4.33 (m, 4H), 4.24- |
| | 4.20 (m, 1H), 3.37 (t, J = 6.0 Hz, 2H), 3.17-3.16 (m, 2H), 2.67 (s, 3H), 2.16-2.14 (m, 2H), |
| | 2.01-1.98 (m, 1H), 1.88-1.83 (m, 3H), 1.75-1.73 (m, 2H), 1.66 (d, J = 6.4 Hz, 3H), 1.35-1.33 |
| | (m, 2H) |
| I-16 | 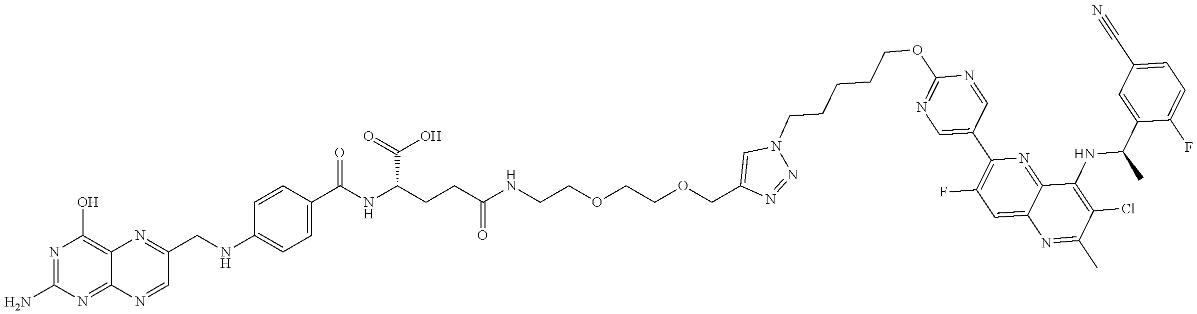 [SMILES: Cc1nc2cc(F)c(-c3cnc(OCCCCCn4cc(COCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| | N2-(4-(((2-amino-4-hydroxypteridin-6-yl)methyl)amino)benzoyl)-N5-(2-(2-((1-(5-((5-(7- |
| | chloro-8-((I-1-(5-cyano-2-fluorophenyl)ethyl)amino)-3-fluoro-6-methyl-1,5-naphthyridin- |
| | 2-yl)pyrimidin-2-yl)oxy)pentyl)-1H-1,2,3-triazol-4-yl)methoxy)ethoxy)ethyl)-L- |
| | glutamine (Compound I-16) |
| | LCMS m/z 1130.99 [M + 1]⁺ |
| | ¹H NMR (400 MHz, DMSO-d6 with D₂O) δ 8.89 (s, 2H), 8.62 (s, 1H), 8.08-8.03 (m, 2H), |
| | 7.90 (d, J = 6.8 Hz, 1H), 7.74-7.72 (m, 1H), 7.58 (d, J = 8.8 Hz, 2H), 7.23 (t, J = 9.6 Hz, 1H), |
| | 6.60 (d, J = 8.4 Hz, 2H), 6.50 (d, J = 7.2 Hz, 1H), 4.46 (s, 4H), 4.35 (d, J = 6.4 Hz, 4H), 4.23- |
| | 4.20 (m, 1H), 3.46 (d, J = 9.6 Hz, 4H), 3.32 (t, J = 5.2 Hz, 2H), 3.13 (t, J = 6.8 Hz, 2H), 2.69 |
| | (s, 3H), 2.16-2.15 (m, 2H), 2.06-1.99 (m, 1H), 1.88-1.84 (m, 3H), 1.78-1.76 (m, 2H), 1.66 (d, J = |
| | 6.8 Hz, 3H), 1.36-1.34 (m, 2H) |
| I-17 | 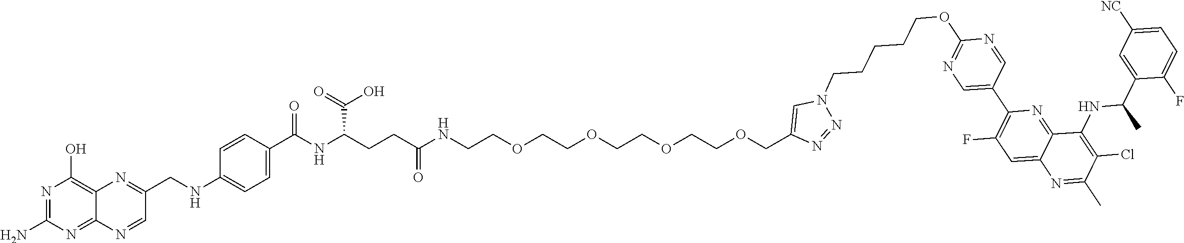 [SMILES: Cc1nc2cc(F)c(-c3cnc(OCCCCCn4cc(COCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| | (S)-18-(4-(((2-amino-4-hydroxypteridin-6-yl)methyl)amino)benzamido)-1-(1-(5-((5-(7- |
| | chloro-8-((I-1-(5-cyano-2-fluorophenyl)ethyl)amino)-3-fluoro-6-methyl-1,5-naphthyridin- |
| | 2-yl)pyrimidin-2-yl)oxy)pentyl)-1H-1,2,3-triazol-4-yl)-15-oxo-2,5,8,11-tetraoxa-14- |
| | azanonadecan-19-oic acid (Compound I-17) |
| | LCMS m/z 1218.6 [M + 1]+ |
| | ¹H NMR (400 MHz, DMSO-d6 with D₂O) δ 8.85 (s, 2H), 8.62 (s, 1H), 8.06-8.02 (m, 2H), |
| | 7.87 (d, J = 4.8 Hz, 1H), 7.73-7.70 (m, 1H), 7.57 (d, J = 8.8 Hz, 2H), 7.22 (t, J = 9.6 Hz, 1H), |
| | 6.59 (d, J = 8.8 Hz, 2H), 6.52 (d, J = 6.8 Hz, 1H), 4.46 (s, 4H), 4.35-4.32 (m, 4H), 4.24-4.20 |
| | (m, 1H), 3.49-3.44 (m, 4H), 3.41 (d, J = 5.6 Hz, 8H), 3.31 (t, J = 6.0 Hz, 2H), 3.13-3.12 (m, |
| | 2H), 2.67 (s, 3H), 2.16 (t, J = 6.8 Hz, 2H), 1.99-1.98 (m, 1H), 1.86 (t, J = 6.8 Hz, 3H), 1.77 (t, |
| | J = 6.4 Hz, 2H), 1.66 (d, J = 6.4 Hz, 3H), 1.37-1.35 (m, 2H) |
| I-18 | 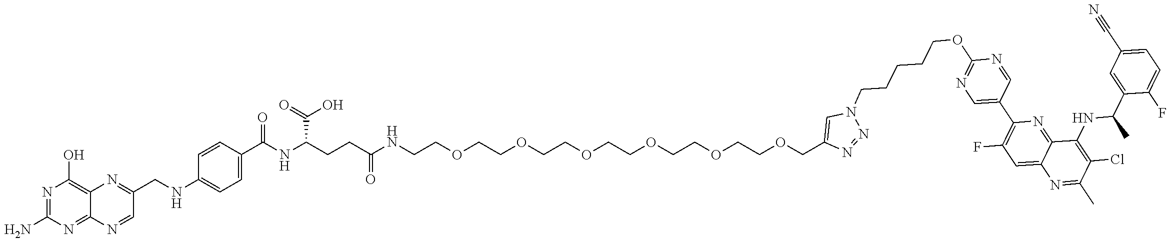 [SMILES: Cc1nc2cc(F)c(-c3cnc(OCCCCCn4cc(COCCOCCOCCOCCOCCOCCNC(=O)CC[C@H](NC(=O)c5ccc(NCc6cnc7nc(N)nc(O)c7n6)cc5)C(=O)O)nn4)nc3)nc2c(N[C@H](C)c2cc(C#N)ccc2F)c1Cl] |
| | (S)-24-(4-(((2-amino-4-hydroxypteridin-6-yl)methyl)amino)benzamido)-1-(1-(5-((5-(7- |
| | chloro-8-((I-1-(5-cyano-2-fluorophenyl)ethyl)amino)-3-fluoro-6-methyl-1,5-naphthyridin- |
| | 2-yl)pyrimidin-2-yl)oxy)pentyl)-1H-1,2,3-triazol-4-yl)-21-oxo-2,5,8,11,14,17-hexaoxa-20- |
| | azapentacosan-25-oic acid (Compound I-18) |
| | LCMS m/z 654.0 [M + 2]++ |
| | ¹H NMR (400 MHz, DMSO-d6 with D₂O) δ 8.79 (s, 2H), 8.61 (s, 1H), 8.02 (d, J = 12.0 Hz, |
| | 2H), 7.83 (d, J = 12.0 Hz, 1H), 7.69 (bs, 1H), 7.55 (d, J = 8.0 Hz, 2H), 7.19 (t, J = 9.6 Hz, 1H), |
| | 6.58 (d, J = 8.0 Hz, 3H), 4.45 (s, 4H), 4.34 (bs, 4H), 4.22-4.20 (m, 1H), 3.47-3.45 (m, 20H), |
| | 3.31 (bs, 2H), 3.12 (bs, 2H), 2.68 (s, 3H), 2.17-2.15 (m, 2H), 1.99 (bs, 1H), 1.86-1.85 (m, 3H), |
| | 1.74-1.72 (m, 2H), 1.66 (d, J = 6.4 Hz, 3H), 1.32 (bs, 2H) |

### 6.1.9. Example 9: Compound I-19

### (S)-30-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-27-oxo-2,5,8,11,14,17,20,23-octaoxa-26-azahentriacontan-31-oic acid (Compound I-19)

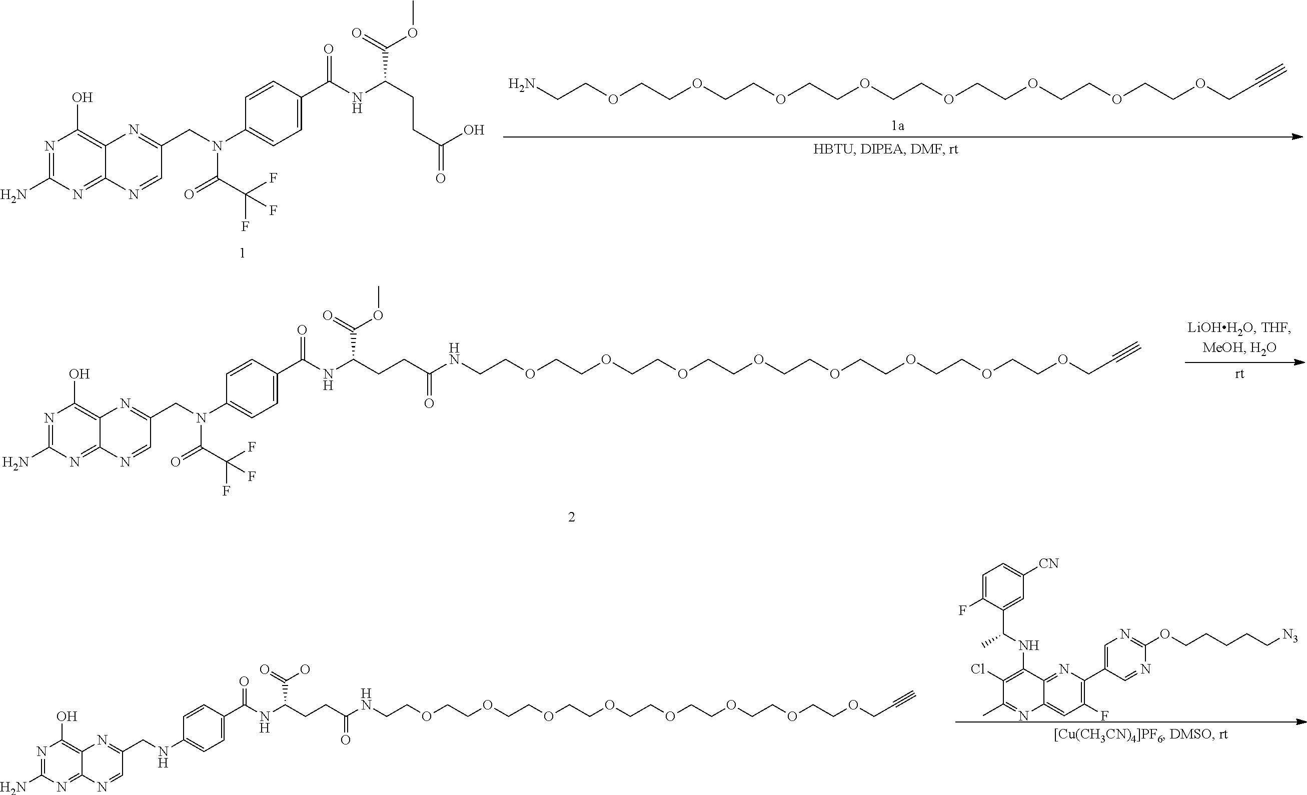 [SMILES: COC(=O)[C@H](CCC(=O)O)NC(=O)c1ccc(N(Cc2cnc3nc(N)nc(O)c3n2)C(=O)C(F)(F)F)cc1 ; C#CCOCCOCCOCCOCCOCCOCCOCCOCCN ; C#CCOCCOCCOCCOCCOCCOCCOCCOCCNC(=O)CC[C@H](NC(=O)c1ccc(N(Cc2cnc3nc(N)nc(O)c3n2)C(=O)C(F)(F)F)cc1)C(=O)OC ; C#CCOCCOCCOCCOCCOCCOCCOCCOCCNC(=O)CC[C@H](NC(=O)c1ccc(NCc2cnc3nc(N)nc(O)c3n2)cc1)C(=O)O ; Cc1nc2cc(F)c(-c3cnc(OCCCCCN=[N+]=[N-])nc3)nc2c(N[C@H](C)c2cc(C#N)ccc2F)c1Cl]

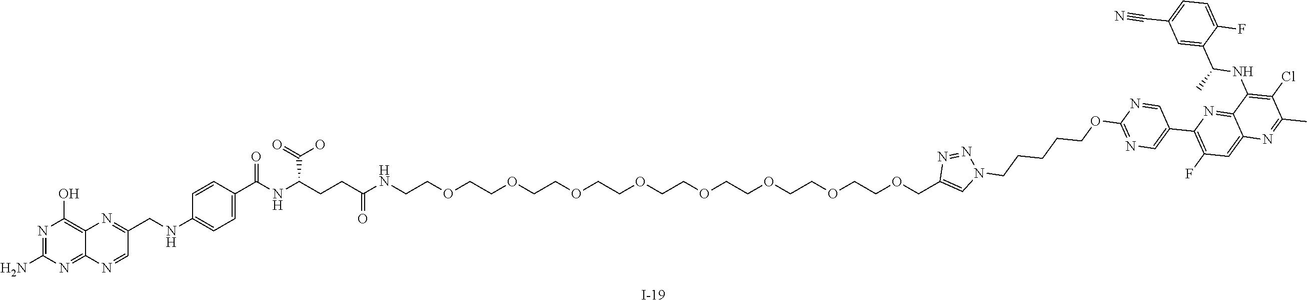

To a solution of (S)-4-(4-(N-((2-amino-4-hydroxypteridin-6-yl)methyl)-2,2,2-trifluoroacetamido)benzamido)-5-methoxy-5-oxopentanoic acid (1, 1.0 eq, 0.31 g, 0.562 mmol) in N,N-dimethylformamide (6 mL), 3,6,9,12,15,18,21,24-octaoxaheptacos-26-yn-1-amine (1a, 1.0 eq, 0.229 g, 0.562 mmol), 2-(1H-benzotriazol-1-yl)-1,1,3,3-tetramethyluronium hexafluorophosphate (HBTU) (1.2 eq, 0.266 g, 0.674 mmol) and N,N-diisopropylethyl amine (1.5 eq, 0.14 mL, 0.843 mmol) were added. The reaction mixture was stirred at room temperature under nitrogen atmosphere in dark (covered with aluminum foil) for 16 h. After the completion of reaction, reaction mixture was purified with reverse phase column chromatography (using 40 g C-18 spherical column eluting 30-60% acetonitrile in water). The desired fractions were concentrated under reduced pressure to afford methyl (S)-32-(4-(N-((2-amino-4-hydroxypteridin-6-yl)methyl)-2,2,2-trifluoroacetamido)benzamido)-29-oxo-4,7,10,13,16,19,22,25-octaoxa-28-azatritriacont-1-yn-33-oate (2) as a yellow solid. Yield 0.320 g, 60%; LCMS m/z 941.28 [M+1]⁺.

To a stirred solution of methyl (S)-32-(4-(N-((2-amino-4-hydroxypteridin-6-yl)methyl)-2,2,2-trifluoroacetamido)benzamido)-29-oxo-4,7,10,13,16,19,22,25-octaoxa-28-azatritriacont-1-yn-33-oate (2, 1.0. eq, 0.250 g, 0.265 mmol) in tetrahydrofuran:methanol:water (1:1:1:) (6 mL), lithium hydroxide monohydrate (10.0 eq, 0.11 g, 2.65 mmol) was added and reaction mixture was stirred for at room temperature 16 h. After completion, reaction mixture was concentrated to get crude. The crude was purified by prep-HPLC using 20-60% acetonitrile in water with 0.1% trifluoroacetic acid). The desired fractions were lyophilized to afford (S)-32-(4-(((2-amino-4-hydroxypteridin-6-yl)methyl)amino)benzamido)-29-oxo-4,7,10,13,16,19,22,25-octaoxa-28-azatritriacont-1-yn-33-oic acid as a yellow solid. Yield 0.092 g, 41%; LCMS m/z 831.19 [M+1]⁺.

To a solution of (S)-32-(4-(((2-amino-4-hydroxypteridin-6-yl)methyl)amino)benzamido)-29-oxo-4,7,10,13,16,19,22,25-octaoxa-28-azatritriacont-1-yn-33-oic acid (1.0 eq, 0.044 g, 0.053 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.05 eq, 0.031 g, 0.055 mmol) in dimethylsulfoxide (1.5 mL), tetrakis(acetonitrile)copper(I) hexafluorophosphate (2.8 eq, 0.055 g, 0.148 mmol) was added and reaction mixture was stirred at room temperature for 0.5 h. After completion, reaction mixture was quenched with acetic acid (0.15 mL) and directly purified by prep HPLC (eluting from C18 column with 30-70% acetonitrile in water with 0.1% trifluoroacetic acid). Fractions containing the desired product were combined and lyophilized to dryness to afford (S)-30-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-27-oxo-2,5,8,11,14,17,20,23-octaoxa-26-azahentriacontan-31-oic acid (Cpd. No. I-19) as a yellow solid. Yield: 0.035 g, 47%; LCMS m/z 698.2 [M+2]++; ¹H-NMR (400 MHz, DMSO-d6 with D₂O) δ 8.98 (s, 2H), 8.66 (s, 1H), 8.15 (d, J=10.8 Hz, 1H), 8.08 (s, 1H), 7.99-7.97 (m, 1H), 7.79-7.76 (m, 1H), 7.63 (d, J=8.4 Hz, 2H), 7.32-7.27 (m, 1H), 6.63 (d, J=8.8 Hz, 2H), 6.49-6.45 (m, 1H), 4.49 (s, 4H), 4.41-4.36 (m, 4H), 4.27-4.23 (m, 1H), 3.52-3.49 (m, 4H), 3.47-3.45 (m, 24H), 3.36-3.33 (m, 2H), 3.16-3.13 (m, 2H), 2.67 (s, 3H), 2.20-2.16 (m, 2H), 2.10-1.92 (m, 1H), 1.93-1.86 (m, 3H), 1.82-1.78 (m, 2H), 1.68 (d, J=6.8 Hz, 3H), 1.41-1.37 (m, 2H).

### 6.1.10. Example 10: Compound I-20

### (S)-36-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-33-oxo-2,5,8,11,14,17,20,23,26,29-decaoxa-32-azaheptatriacontan-37-oic acid (Compound I-20)

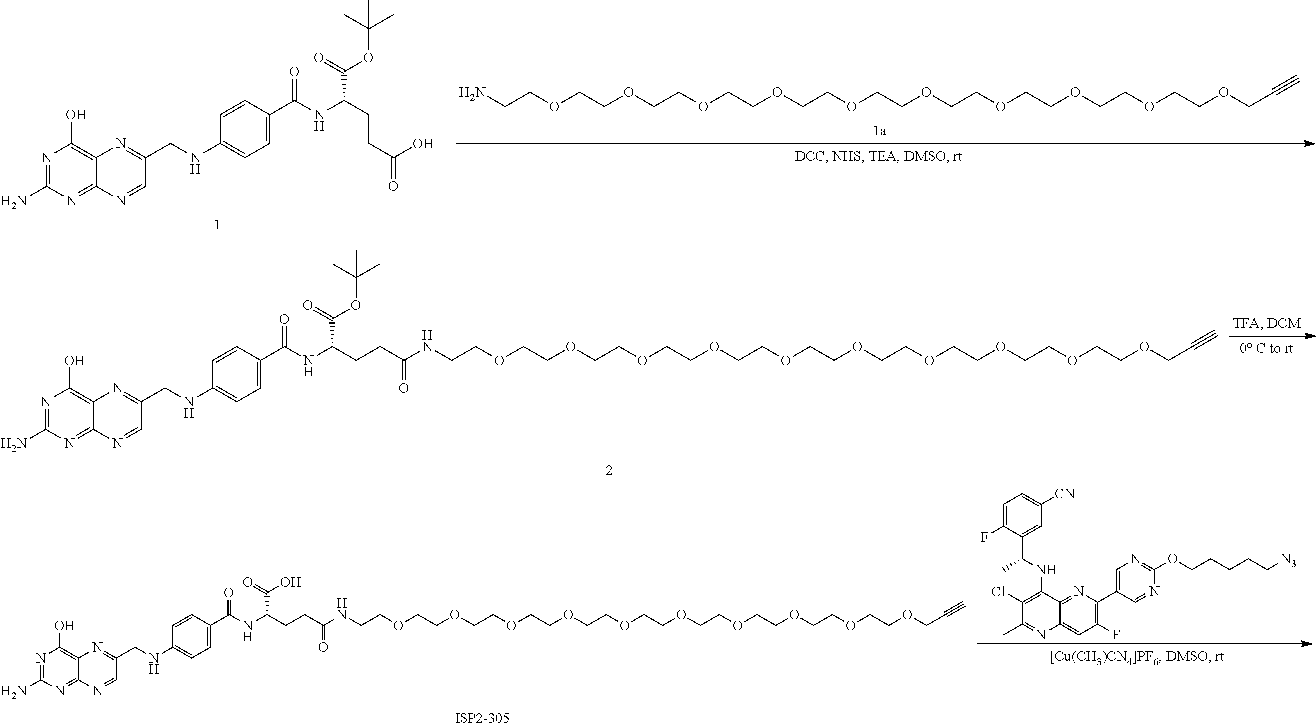 [SMILES: CC(C)(C)OC(=O)[C@H](CCC(=O)O)NC(=O)c1ccc(NCc2cnc3nc(N)nc(O)c3n2)cc1 ; C#CCOCCOCCOCCOCCOCCOCCOCCOCCOCCOCCN ; C#CCOCCOCCOCCOCCOCCOCCOCCOCCOCCOCCNC(=O)CC[C@H](NC(=O)c1ccc(NCc2cnc3nc(N)nc(O)c3n2)cc1)C(=O)OC(C)(C)C ; C#CCOCCOCCOCCOCCOCCOCCOCCOCCOCCOCCNC(=O)CC[C@H](NC(=O)c1ccc(NCc2cnc3nc(N)nc(O)c3n2)cc1)C(=O)O ; Cc1nc2cc(F)c(-c3cnc(OCCCCCN=[N+]=[N-])nc3)nc2c(N[C@H](C)c2cc(C#N)ccc2F)c1Cl]

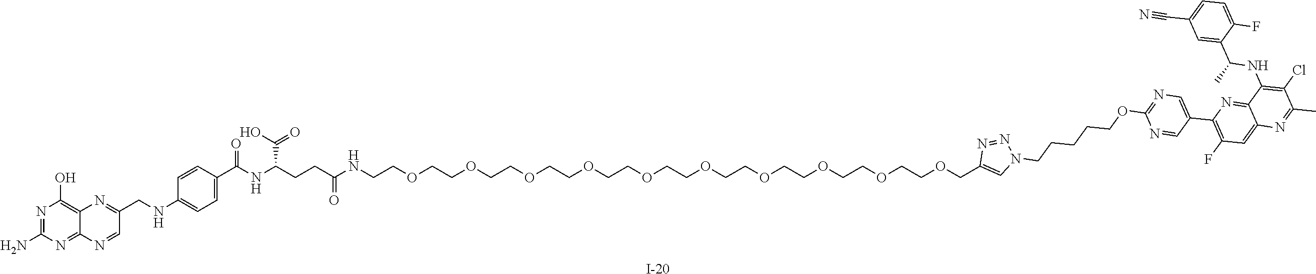

To a solution of (S)-4-(4-(((2-amino-4-hydroxypteridin-6-yl)methyl)amino)benzamido)-5-(tert-butoxy)-5-oxopentanoic acid (1, 1.0 eq, 0.200 g, 0.402 mmol) in dimethylsulfoxide (20 mL), 1-hydroxypyrrolidine-2,5-dione (2.0 eq, 0.0925 g, 0.804 mmol), N,N′-dicyclohexylmethanediimine (2.0 eq, 0.166 g, 0.804 mmol) were added under nitrogen atmosphere in dark (covered with Aluminium foil). The reaction mixture was stirred at room temperature for 16 h. After the completion of reaction, reaction mixture was filtered through sintered glass funnel. To the filtrate solution, 3,6,9,12,15,18,21,24,27,30-decaoxatritriacont-32-yn-1-amine (1a, 1.0 eq, 0.20 g, 0.402 mmol) in dimethylsulfoxide (2 mL) followed by triethyl amine (2.0 eq, 0.11 mL, 0.804 mmol) were added. The reaction mixture was stirred at 35° C. for 16 h. After the completion of reaction, reaction mixture was purified with reverse phase column chromatography (using 40 g C-18 spherical column eluting 30-60% acetonitrile in water). The desired fractions were concentrated under reduced pressure to afford tert-butyl (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oate (2) as a yellow solid. Yield 0.200 g, 51%; LCMS m/z 975.62 [M+1]⁺.

To a stirred solution of tert-butyl (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oate (2, 1.0 eq, 0.20 g, 0.205 mmol) in dichloromethane (10 mL) at 0° C., trifluroacetic acid (2.0 mL) was added and reaction mixture was stirred at room temperature for 3 h. After completion, reaction mixture was concentrated. The crude was purified by prep-HPLC using 20-60% acetonitrile in water with 0.1% triethylamine. The desired fractions were lyophilized to afford (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oic acid as a yellow solid. Yield 0.025 g, 13.6%; LCMS m/z 919.70 [M+1]⁺.

To a solution of (S)-38-(4-(((2-amino-4-hydroxypteridin-6-yl)methyl)amino)benzamido)-35-oxo-4,7,10,13,16,19,22,25,28,31-decaoxa-34-azanonatriacont-1-yn-39-oic acid (1.0 eq, 0.023 g, 0.025 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.01 eq, 0.014 g, 0.025 mmol) in dimethylsulfoxide (1.5 mL), tetrakis(acetonitrile)copper(I) hexafluorophosphate (2.8 eq, 0.026 g, 0.070 mmol) was added and reaction mixture was stirred at room temperature for 0.5 h. After completion, reaction mixture was quenched with acetic acid (0.15 mL) and directly purified by prep HPLC (30-70% acetonitrile in water with 0.1% trifluoroacetic acid). Fractions containing the desired product were combined and lyophilized to dryness to afford (S)-36-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-33-oxo-2,5,8,11,14,17,20,23,26,29-decaoxa-32-azaheptatriacontan-37-oic acid (Cpd. No. I-20) as a yellow solid. Yield: 0.013 g, 35%; LCMS m/z 742.25 [M+2]++; ¹H NMR (400 MHz, DMSO-d6 with D₂O) δ8.81 (s, 2H), 8.58 (s, 1H), 7.79 (d, J=14.8 Hz, 2H), 7.80 (d, J=5.6 Hz, 1H), 7.65 (t, J=4.4 Hz, 1H), 7.54 (d, J=8.4 Hz, 2H), 7.16 (t, J=10.0 Hz, 1H), 6.58 (d, J=8.8 Hz, 2H), 6.43 (t, J=5.6 Hz, 1H), 4.44 (d, J=2.0 Hz, 4H), 4.33 (d, J=6.4 Hz, 4H), 3.47-3.40 (m, 35H), 3.31 (t, J=9.2 Hz, 2H), 3.13 (d, J=5.2 Hz, 2H), 2.64 (d, J=16.4 Hz, 3H), 2.17 (t, J=7.2 Hz, 2H), 2.01-1.99 (m, 2H), 1.93-1.85 (m, 4H), 1.75-1.72 (m, 2H), 1.63-1.62 (m, 3H), 1.39-1.31 (m, 2H).

### 6.1.11. Example 11: Compound I-21

### (S)-16-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Compound I-21)

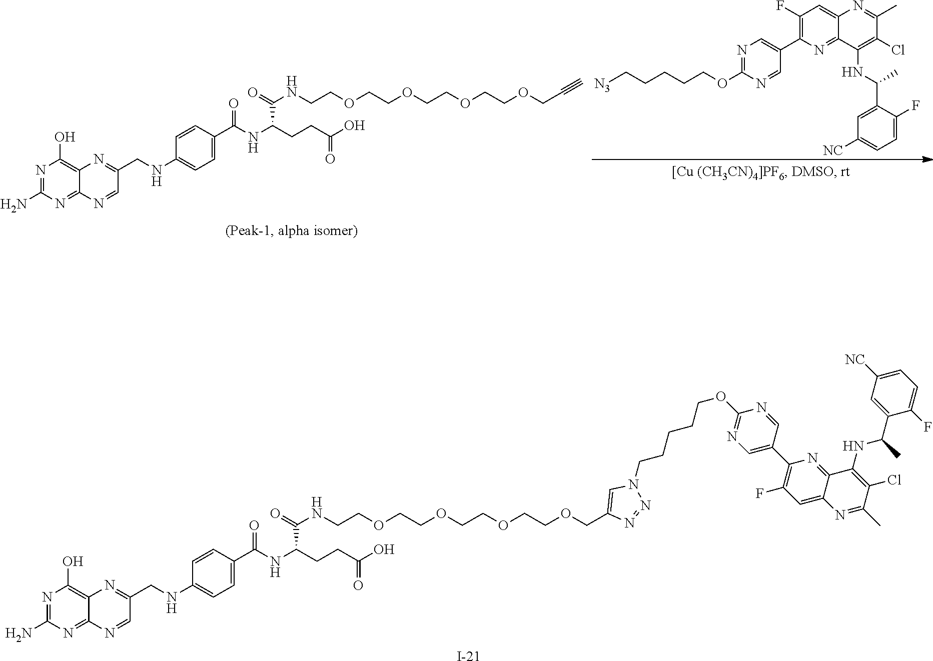

To a solution of (S)-18-(4-(((2-amino-4-hydroxypteridin-6-yl)methyl)amino)benzamido)-17-oxo-4,7,10,13-tetraoxa-16-azahenicos-1-yn-21-oic acid (Peak-1, alpha isomer, 1.0 eq, 0.040 g, 0.061 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.034 g, 0.061 mmol) in dimethylsulfoxide (1.0 mL), tetrakis(acetonitrile)copper(I) hexafluorophosphate (2.8 eq, 0.063 g, 0.171 mmol) was added and reaction mixture was stirred at room temperature for 1 h. After completion, reaction mixture was directly purified by prep HPLC (20-35% acetonitrile in water with 0.1% TFA). Fractions containing the desired product were combined and lyophilized to dryness to afford (S)-16-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Cpd. No. I-21) as a yellow solid. Yield: 0.020 g, 27.0%; LCMS m/z 610.0 [M+1]; ¹H NMR (400 MHz, DMSO-d6 with D₂O) δ8.82 (s, 2H), 8.60 (s, 1H), 8.02-8.00 (m, 2H), 7.84 (d, J=7.6 Hz, 1H), 7.69 (bs, 1H), 7.55 (d, J=8.0 Hz, 2H), 7.19 (t, J=9.6 Hz, 1H), 6.57 (d, J=8.8 Hz, 2H), 6.53-6.48 (m, 1H), 4.44 (t, J=10.0 Hz, 3H), 4.35-4.29 (m, 5H), 3.47-3.34 (m, 12H), 3.21-3.09 (m, 3H), 2.66 (s, 3H), 2.23-2.21 (m, 2H), 1.91-1.85 (m, 6H), 1.74 (bs, 2H), 1.65 (d, J=6.4 Hz, 3H), 1.31 (bs, 2H).

### 6.1.12. Example 12: Compound I-22

### Synthesis of rac-(R)-18-(4-(((2-amino-4-hydroxypteridin-6-yl)methyl)amino)benzamido)-1-(1-(2-(2-(4-(5-(3-(2-(difluoromethoxy)benzyl)-2-methylimidazo[1,2-a]pyridin-6-yl)pyridin-2-yl)piperazin-1-yl)ethoxy)ethyl)-1H-1,2,3-triazol-4-yl)-15-oxo-2,5,8,11-tetraoxa-14-azanonadecan-19-oic acid (Compound I-22)

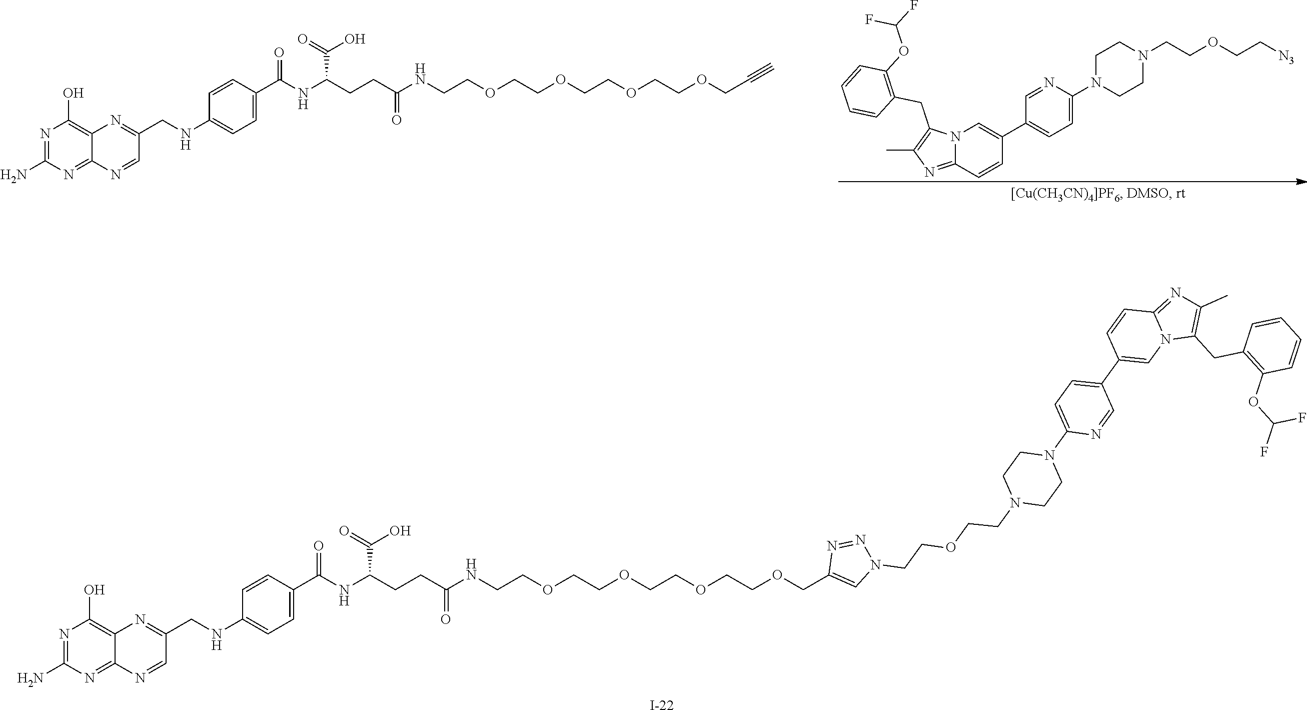

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.030 g, 0.0458 mmol) and 6-(6-(4-(2-(2-azidoethoxy)ethyl)piperazin-1-yl)pyridin-3-yl)-3-(2-(difluoromethoxy)benzyl)-2-methylimidazo[1,2-a]pyridine (1.0 eq, 0.0258 g, 0.0458 mmol) in anhydrous dimethylsulfoxide (1.5 mL), tetrakis(acetonitrile)copper(I) hexafluorophosphate (2.8 eq, 0.0478 g, 0.128 mmol) was added and reaction mixture was stirred at room temperature for 0.5 h. After completion, the reaction mixture was quenched with acetic acid (0.2 mL) and directly purified by prep HPLC (15-35% acetonitrile in water with 0.1% trifluoroacetic acid). All the fractions containing desired compound were combined and lyophilized to afford rac-(R)-18-(4-(((2-amino-4-hydroxypteridin-6-yl)methyl)amino)benzamido)-1-(1-(2-(2-(4-(5-(3-(2-(difluoromethoxy)benzyl)-2-methylimidazo[1,2-a]pyridin-6-yl)pyridin-2-yl)piperazin-1-yl)ethoxy)ethyl)-1H-1,2,3-triazol-4-yl)-15-oxo-2,5,8,11-tetraoxa-14-azanonadecan-19-oic acid (Cpd. No. I-22) as a yellow solid. Yield: 0.017 g, 30.48%; LCMS m/z 1217.97 [M+1]⁺; ¹H NMR (400 MHz, DMSO-d6 with D₂O) δ8.73 (s, 1H), 8.61 (s, 1H), 8.51 (d, J=2.4 Hz, 1H), 8.16 (d, J=9.2 Hz, 1H), 8.08 (s, 1H), 7.97 (dd, J=7.6 and 2.4 Hz, 1H), 7.91 (d, J=9.2 Hz, 1H), 7.60 (d, J=8.4 Hz, 2H), 7.36-6.96 (m, 6H), 6.61 (d, J=8.8 Hz, 2H), 4.56-4.55 (m, 2H), 4.49-4.46 (m, 6H), 4.47-4.32 (m, 1H), 4.26-4.22 (m, 1H), 3.86 (t, J=4.8 Hz, 2H), 3.53-3.52 (m, 2H), 3.47-3.46 (m, 2H), 3.40-3.39 (m, 9H), 3.32-3.29 (m, 4H), 3.25-3.18 (m, 2H), 3.13-3.05 (m, 3H), 3.09-2.95 (m, 3H), 2.39 (s, 3H), 2.18-2.15 (m, 2H), 2.02-2.00 (m, 1H), 1.90-1.86 (m, 1H).

### 6.1.13. Example 13: Compound I-23

### (S)-18-(4-(((2-amino-4-hydroxypteridin-6-yl)methyl)amino)benzamido)-1-(1-(4-(5-(3-chloro-4-((2,5-dimethylphenyl)amino)-2-methylquinolin-6-yl)picolinamido)butyl)-1H-1,2,3-triazol-4-yl)-15-oxo-2,5,8,11-tetraoxa-14-azanonadecan-19-oic acid. (Compound I-23)

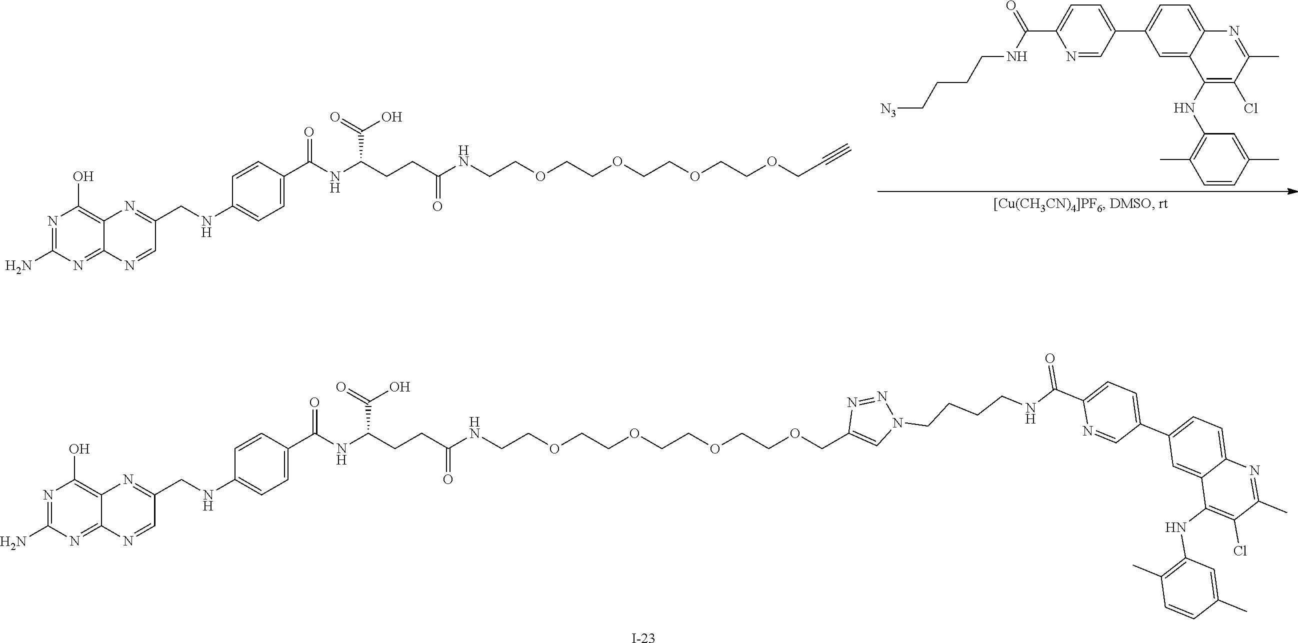

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and N-(4-azidobutyl)-5-(3-chloro-4-((2,5-dimethylphenyl)amino)-2-methylquinolin-6-yl)picolinamide (1.0 eq, 0.0236 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (15-37% acetonitrile in water with 0.1% trifluoroacetic acid). Fractions containing the desired product were combined and lyophilized to dryness to afford (S)-18-(4-(((2-amino-4-hydroxypteridin-6-yl)methyl)amino)benzamido)-1-(1-(4-(5-(3-chloro-4-((2,5-dimethylphenyl)amino)-2-methylquinolin-6-yl)picolinamido)butyl)-1H-1,2,3-triazol-4-yl)-15-oxo-2,5,8,11-tetraoxa-14-azanonadecan-19-oic acid (Cpd. No. I-23) as a yellow solid. Yield: 0.019 g; 35.48%. LCMS: m/z 585.21 [M+2]++; ¹H NMR (400 MHz, DMSO-d6 with D₂O) δ 8.68 (s, 1H), 8.62 (s, 1H), 8.30 (d, J=9.2 Hz, 1H), 8.09 (s, 1H), 8.06-7.99 (m, 3H), 7.94 (d, J=7.2 Hz, 1H), 7.60 (d, J=7.2 Hz, 2H), 7.30 (d, J=8 Hz, 1H), 7.21 (d, J=7.6 Hz, 1H), 7.09 (s, 1H), 6.60 (d, J=8.4 Hz, 2H), 4.46 (s, 4H), 4.35 (t, J=6.8 Hz, 2H), 4.25-4.21 (m, 1H), 3.48 (d, J=7.6 Hz, 4H), 3.42-3.41 (m, 8H), 3.32-3.30 (m, 4H), 3.13-3.11 (m, 2H), 2.75 (s, 3H), 2.24 (s, 3H), 2.17-2.14 (m, 5H), 2.03-2.00 (m, 2H), 1.90-1.81 (m, 4H), 1.49-1.47 (m, 2H).

### 6.1.14. Example 14: Compound I-24

### (S)-18-(4-(((2-amino-4-hydroxypteridin-6-yl)methyl)amino)benzamido)-1-(1-(2-(2-((5-(1-(2-(difluoromethoxy)benzyl)-2-methyl-1H-benzo[d]imidazol-6-yl)pyrimidin-2-yl)oxy)ethoxy)ethyl)-1H-1,2,3-triazol-4-yl)-15-oxo-2,5,8,11-tetraoxa-14-azanonadecan-19-oic acid (Compound I-24)

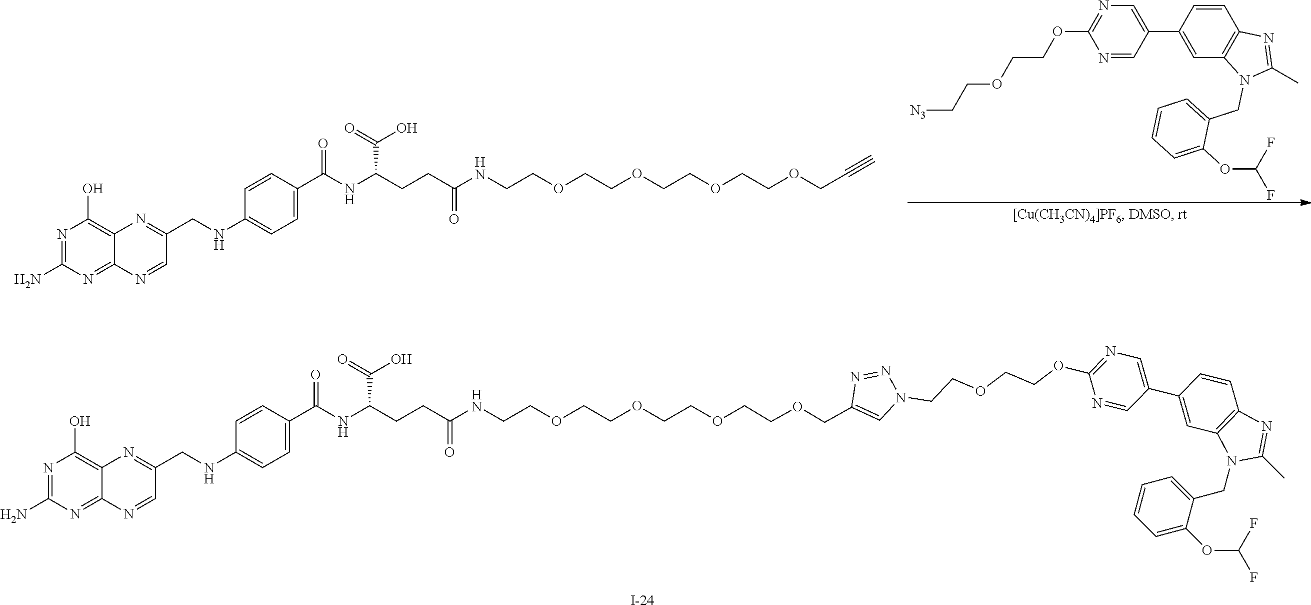

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and 6-(2-(2-(2-azidoethoxy)ethoxy)pyrimidin-5-yl)-1-(2-(difluoromethoxy)benzyl)-2-methyl-1H-benzo[d]imidazole (1.0 eq, 0.0227 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (20-42% acetonitrile in water with 0.1% trifluoroacetic acid). Fractions containing the desired product were combined and lyophilized to dryness to afford (S)-18-(4-(((2-amino-4-hydroxypteridin-6-yl)methyl)amino)benzamido)-1-(1-(2-(2-((5-(1-(2-(difluoromethoxy)benzyl)-2-methyl-1H-benzo[d]imidazol-6-yl)pyrimidin-2-yl)oxy)ethoxy)ethyl)-1H-1,2,3-triazol-4-yl)-15-oxo-2,5,8,11-tetraoxa-14-azanonadecan-19-oic acid (Cpd. No. I-24) as a yellow solid. Yield: 0.023 g; 43.66%; LC-MS; m/z 575.98 [M+2]”; ¹H NMR (400 MHz, DMSO-d6 with D₂O) δ 8.90 (s, 2H), 8.64 (s, 1H), 8.13 (s, 1H), 8.01 (s, 1H), 7.87 (s, 2H), 7.61 (d, J=8.4 Hz, 2H), 7.45-7.05 (m, 5H), 6.62 (d, J=8.8 Hz, 2H), 5.73 (s, 2H), 4.51-4.44 (m, 8H), 4.24-4.23 (m, 1H), 3.86 (bs, 2H), 3.76 (bs, 2H), 3.47-3.41 (m, 13H), 3.32-3.30 (m, 2H), 3.13-3.11 (m, 2H), 2.78 (s, 3H), 2.18-2.16 (m, 2H), 2.10-2.00 (m, 1H), 1.90-1.88 (m, 1H).

### 6.1.15. Example 15: Compound I-25

### (S)-18-(4-(((2-amino-4-hydroxypteridin-6-yl)methyl)amino)benzamido)-1-(1-(5-((5-(1-(2-(difluoromethoxy)benzyl)-2-((3-(2-oxopyrrolidin-1-yl)phenoxy)methyl)-1H-benzo[d]imidazol-6-yl)pyridin-2-yl)oxy)pentyl)-1H-1,2,3-triazol-4-yl)-15-oxo-2,5,8,11-tetraoxa-14-azanonadecan-19-oic acid (Compound I-25)

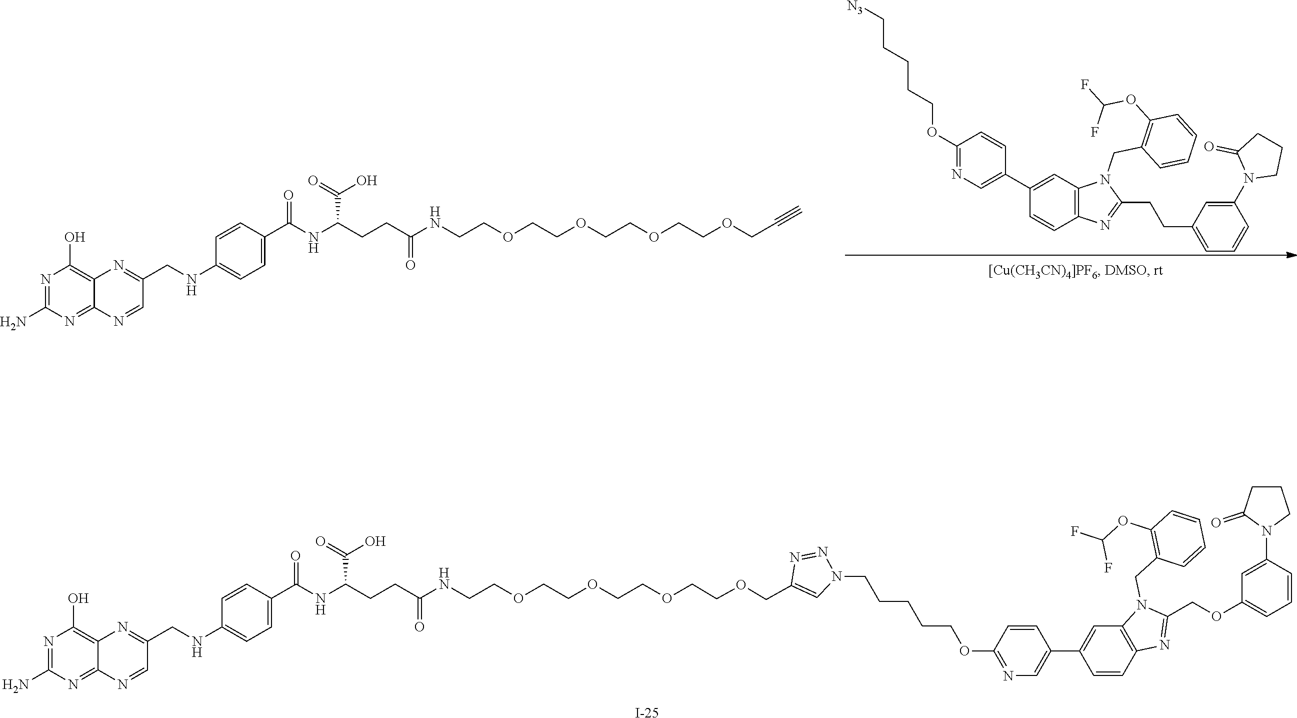

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and 1-(3-((6-(6-((5-azidopentyl)oxy)pyridin-3-yl)-1-(2-(difluoromethoxy)benzyl)-1H-benzo[d]imidazol-2-yl)methoxy)phenyl)pyrrolidin-2-one (1.0 eq, 0.0306 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (25-38% acetonitrile in water with 0.1% trifluoroacetic acid). Fractions containing the desired product were combined and lyophilized to dryness to afford (S)-18-(4-(((2-amino-4-hydroxypteridin-6-yl)methyl)amino)benzamido)-1-(1-(5-((5-(1-(2-(difluoromethoxy)benzyl)-2-((3-(2-oxopyrrolidin-1-yl)phenoxy)methyl)-1H-benzo[d]imidazol-6-yl)pyridin-2-yl)oxy)pentyl)-1H-1,2,3-triazol-4-yl)-15-oxo-2,5,8,11-tetraoxa-14-azanonadecan-19-oic acid (Cpd. No. I-25) as a yellow solid. Yield: 0.032 g; 52%; LCMS; m/z 662.28 [M+2]++; ¹H NMR (400 MHz, DMSO-d6 with D₂O) δ 8.67 (s, 1H), 8.40 (s, 1H), 8.04 (s, 1H), 7.97 (dd, J=8.8 and 2.4 Hz, 1H), 7.82 (d, J=8.4 Hz, 2H), 7.66-7.61 (m, 3H), 7.38-7.01 (m, 7H), 6.95 (d, J=7.6 Hz, 1H), 6.86 (d, J=8.4 Hz, 1H), 6.74 (d, J=8.4 Hz, 1H), 6.62 (d, J=8.8 Hz, 2H), 5.71 (s, 2H), 5.48 (s, 2H), 4.49 (d, J=14.0 Hz, 4H), 4.33 (t, J=6.8 Hz, 2H), 4.26-4.21 (m, 3H), 3.76-3.74 (m, 2H), 3.49-3.47 (m, 4H), 3.43-3.42 (m, 8H), 3.32 (t, J=5.6 Hz, 2H), 3.13 (t, J=5.6 Hz, 2H), 2.17 (t, J=6.8 Hz, 2H), 2.03 (t, J=7.6 Hz, 3H), 1.86-1.82 (m, 3H), 1.73-1.69 (m, 2H), 1.34-1.32 (m, 2H).

### 6.1.16. Example 16: Compound I-26

### (S)-18-(4-(((2-amino-4-hydroxypteridin-6-yl)methyl)amino)benzamido)-1-(1-(6-(4-(3-(2,5-dichlorobenzyl)-2-methylimidazo[1,2-a]pyridin-6-yl)-1H-pyrazol-1-yl)hexyl)-1H-1,2,3-triazol-4-yl)-15-oxo-2,5,8,11-tetraoxa-14-azanonadecan-19-oic acid (Compound I-26)

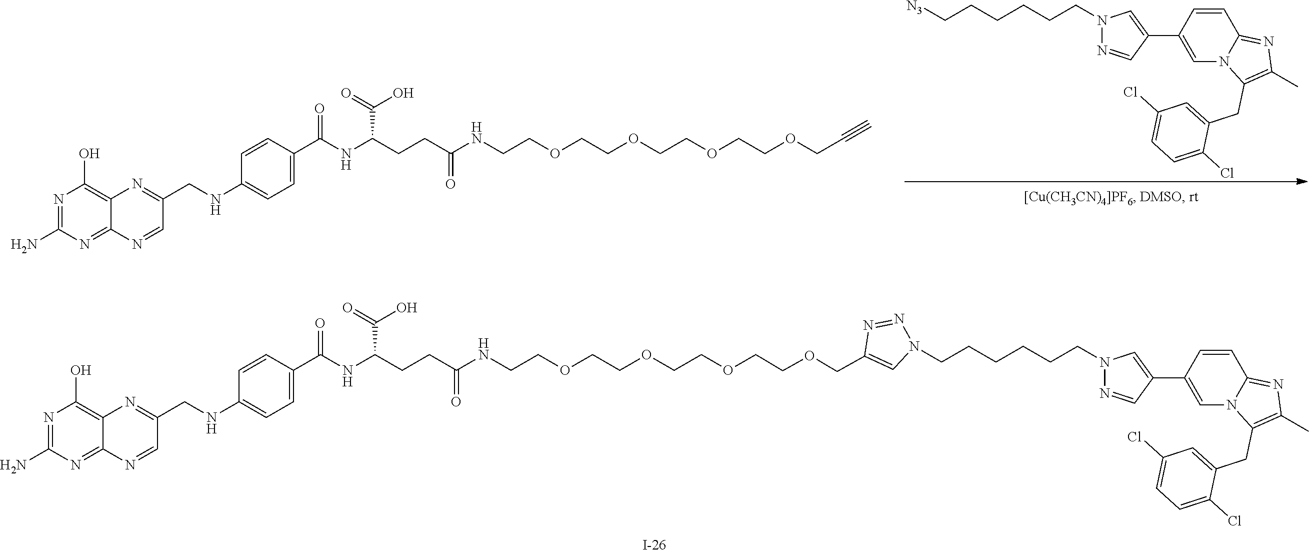

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.03 g, 0.0458 mmol) and 6-(1-(6-azidohexyl)-1H-pyrazol-4-yl)-3-(2,5-dichlorobenzyl)-2-methylimidazo[1,2-a]pyridine (1.0 eq, 0.022 g, 0.0458 mmol) in dimethylsulfoxide (1.5 mL), tetrakis(acetonitrile)copper(I) hexafluorophosphate (2.8 eq, 0.0478 g, 0.128 mmol) was added. The reaction mixture was stirred at room temperature for 1 h and reaction mixture was directly purified by prep HPLC (15-35% acetonitrile in water with 0.1% TFA). Fractions containing the desired product were combined and lyophilized to dryness to afford (S)-18-(4-(((2-amino-4-hydroxypteridin-6-yl)methyl)amino)benzamido)-1-(1-(6-(4-(3-(2,5-dichlorobenzyl)-2-methylimidazo[1,2-a]pyridin-6-yl)-1H-pyrazol-1-yl)hexyl)-1H-1,2,3-triazol-4-yl)-15-oxo-2,5,8,11-tetraoxa-14-azanonadecan-19-oic acid (Cpd. No. I-26) as a yellow solid. Yield: 0.033 g; 63%: LCMS; m/z 569.18 [M+2]++; ¹H NMR (400 MHz, DMSO-d6 with D₂O) δ8.79 (s, 1H), 8.65 (s, 1H), 8.36 (s, 1H), 8.19 (d, J=7.4 Hz, 1H), 8.15 (d, J=9.2 Hz, 1H), 8.05 (d, J=8.4 Hz, 2H), 7.96 (d, J=9.2 Hz, 1H), 7.89 (t, J=5.6 Hz, 1H), 7.64 (d, J=8.8 Hz, 2H), 7.57 (d, J=8.4 Hz, 1H), 7.41 (dd, J=8.4 & 2.4 Hz, 1H), 7.34 (d, J=2.4H, 2H), 7.09 (brs, 1H), 6.62 (d, J=8.4H, 1H), 4.55 (s, 2H), 4.48 (s, 4H), 4.32-4.26 (m, 5H), 4.11-4.09 (m, 3H), 3.52-3.50 (m, 4H), 3.36-3.33 (m, 2H), 3.17-3.13 (m, 2H), 2.33 (s, 3H), 2.20-2.16 (m, 2H), 2.05-2.01 (m, 1H), 1.91-1.86 (m, 1H), 1.79-1.75 (m, 4H), 1.24-1.23 (m, 4H).

### 6.1.17. Example 17: Compound I-27

### (S)-2-(4-(((2-amino-4-hydroxypteridin-6-yl)methyl)amino)benzamido)-4-(5-(1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-2,5,8,11-tetraoxatridecan-13-yl)-1,3,4-oxadiazol-2-yl)butanoic acid (Compound I-27)

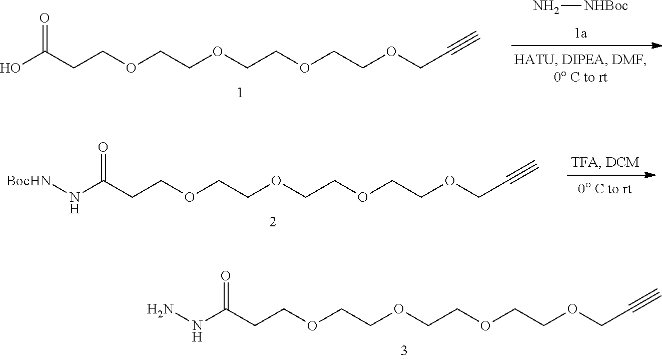 [SMILES: CC(C)(C)OC(=O)NN ; C#CCOCCOCCOCCOCCC(=O)O ; C#CCOCCOCCOCCOCCC(=O)NNC(=O)OC(C)(C)C ; C#CCOCCOCCOCCOCCC(=O)NN]

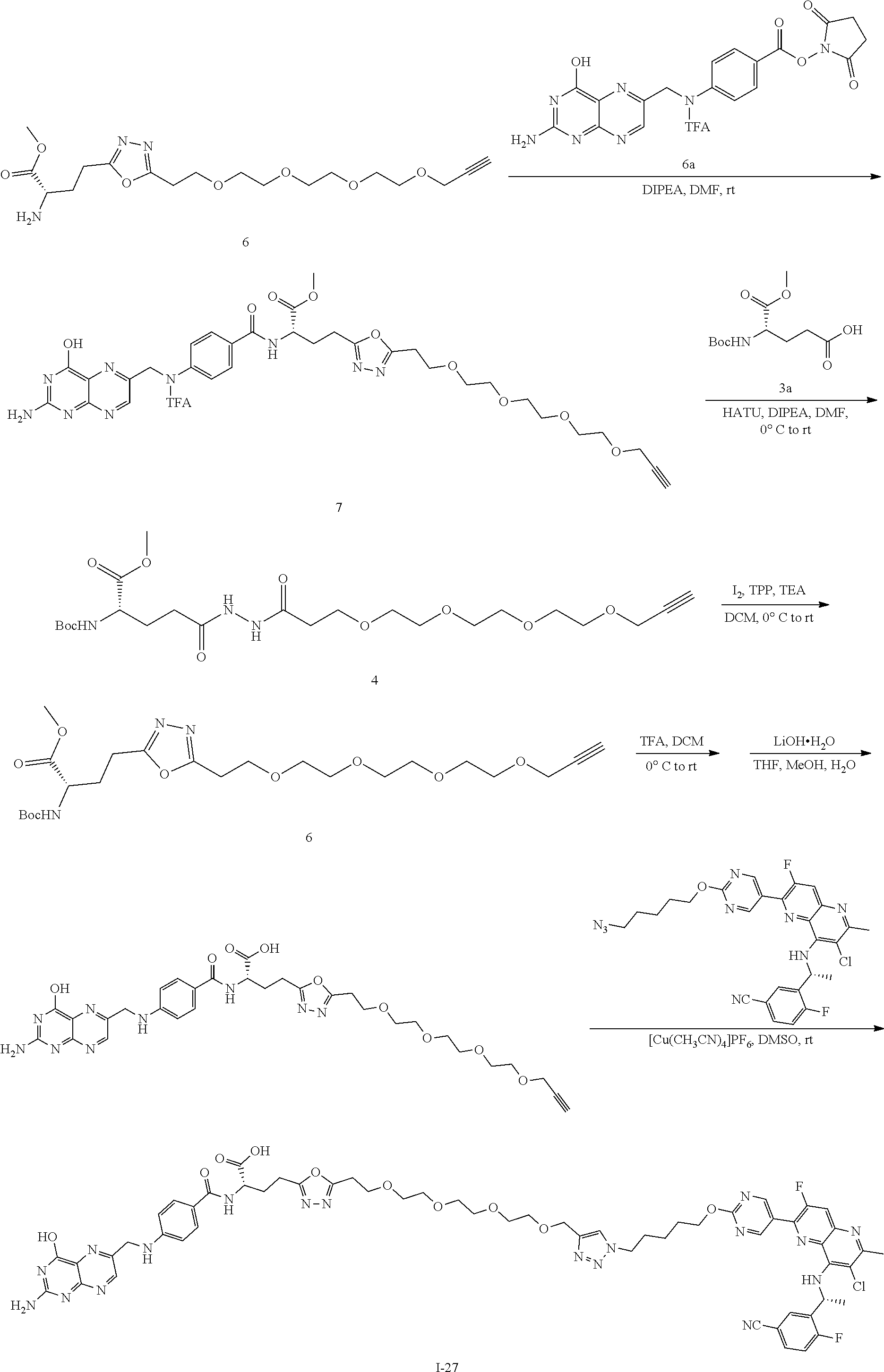

A solution of 4,7,10,13-tetraoxahexadec-15-ynoic acid (1, 1.0 eq, 1.0 g, 3.84 mmol) and tert-butyl hydrazinecarboxylate (1a, 1.0 eq, 0.762 g, 5.76 mmol) in N,N-dimethylformamide (20 mL) was cooled at 0° C., N,N-diisopropylethylamine (3.0 eq, 2.13 mL, 11.5 mmol) and 1-[bis(dimethylamino)methylene]-1H-1,2,3-triazolo[4,5-b]pyridinium 3-oxid hexafluorophosphate (HATU) (1.5 eq, 2.19 g, 5.76 mmol) were added and the reaction mixture was stirred at room temperature for 16 h. After completion, water was added to the reaction mixture and product extracted with ethyl acetate. The organic layer was washed with water, dried over anhydrous sodium sulfate, filtered and concentrated to get crude material which was purified by column chromatography using silica gel (100-200 mesh) and 0-6% methanol in dichloromethane to afford tert-butyl 4-oxo-7,10,13,16-tetraoxa-2,3-diazanonadec-18-ynoate (2) as a light brown viscous liquid. Yield: 0.780 g, 54.2%; LCMS m/z 375.2[M+1]⁺.

A solution of tert-butyl 4-oxo-7,10,13,16-tetraoxa-2,3-diazanonadec-18-ynoate (2, 1.0 eq, 0.780 g, 2.08 mmol) in dichloromethane (4 mL) was cooled at 0° C. then trifluoroacetic acid (4 mL) was added and the reaction mixture was stirred at room temperature for 3 h. After that, the reaction mixture was concentrated, azeotroped with dichloromethane (2-3 times) and dried to afford 4,7,10,13-tetraoxahexadec-15-ynehydrazide (3) as a light brown viscous liquid. Yield: 0.950 g (crude); LCMS m/z 275.0 [M+1]⁺.

A solution of (S)-4-((tert-butoxycarbonyl)amino)-5-methoxy-5-oxopentanoic acid (3a, 1.0 eq, 0.150 g, 0.574 mmol) in NN-dimethylformamide (2 mL) was cooled at 0° C., 4,7,10,13-tetraoxahexadec-15-ynehydrazide (3, 1.1 eq, 0.173 g, 0.632 mmol), N,N-diisopropylethylamine (3.0 eq, 0.31 mL, 1.72 mmol) and 1-[bis(dimethylamino)methylene]-1H-1,2,3-triazolo[4,5-b]pyridinium 3-oxid hexafluorophosphate (HATU) (1.5 eq, 0.327 g, 0.861 mmol) were added and the reaction mixture was stirred at room temperature for 16 h. After completion, water was added to the reaction mixture and products extracted with ethyl acetate. The organic layer was washed with water, dried over anhydrous sodium sulfate, filtered and concentrated to get crude material which was purified by column chromatography using silica gel (100-200 mesh) and 04% methanol in dichloromethane to afford methyl (S)-22-((tert-butoxycarbonyl)amino)-16,19-dioxo-4,7,10,13-tetraoxa-17,18-diazatricos-1-yn-23-oate (4) as a colorless viscous liquid. Yield: 0.085 g, 26.5%; LCMS m/z 518.2[M+1]⁺.

A solution of triphenylphosphine (2.0 eq, 0.203 g, 0.773 mmol) and iodine (2.0 eq, 0.196 g, 0.773 mmol) in dichloromethane (2 mL) was stirred at room temperature for 10 minutes. Then, the solution was cooled to 0° C. before adding triethylamine (4.0 eq, 0.22 mL, 1.55 mmol) and the reaction was again warmed and stirred at room temperature for 10 minutes. A solution of methyl (S)-22-((tert-butoxycarbonyl)amino)-16,19-dioxo-4,7,10,13-tetraoxa-17,18-diazatricos-1-yn-23-oate (4, 1.0 eq, 0.200 g, 0.386 mmol) in dichloromethane (2 mL) was added at 0° C. and the reaction mixture was warmed and stirred at room temperature for 1 h. After completion, water was added to the reaction mixture and products extracted with dichloromethane. The organic layer was dried over anhydrous sodium sulfate, filtered and concentrated to get crude which was purified by column chromatography using silica gel (100-200 mesh) and 0-5% methanol in dichloromethane to afford methyl (S)-4-(5-(3,6,9,12-tetraoxapentadec-14-yn-1-yl)-1,3,4-oxadiazol-2-yl)-2-((tert-butoxycarbonyl)amino)butanoate (5) as a light yellow viscous liquid. Yield: 0.200 g, 94.5%; LCMS m/z 500.2 [M+1]⁺.

A solution of methyl (S)-4-(5-(3,6,9,12-tetraoxapentadec-14-yn-1-yl)-1,3,4-oxadiazol-2-yl)-2-((tert-butoxycarbonyl)amino)butanoate (5, 1.0 eq, 0.190 g, 0.380 mmol) in dichloromethane (1.5 mL) was cooled at 0° C. then trifluoroacetic acid (1.5 mL) was added and the reaction mixture was stirred at room temperature for 3 h. The reaction was then concentrated, azeotroped with dichloromethane (2-3 times), washed with diethyl ether (2-3 times) and dried to afford methyl (S)-4-(5-(3,6,9,12-tetraoxapentadec-14-yn-1-yl)-1,3,4-oxadiazol-2-yl)-2-aminobutanoate (6) as a colorless viscous liquid. Yield: 0.210 g (crude); LCMS m/z 400.2 [M+1]⁺.

To a suspension of 2,5-dioxopyrrolidin-1-yl 4-(N-((2-amino-4-hydroxypteridin-6-yl)methyl)-2,2,2-trifluoroacetamido)benzoate (6a, 1.0 eq) and methyl (S)-4-(5-(3,6,9,12-tetraoxapentadec-14-yn-1-yl)-1,3,4-oxadiazol-2-yl)-2-aminobutanoate (6, 1.2 eq) in N,N-dimethylformamide (10 vol.), N,N-diisopropylethylamine (5.0 eq) is added and the reaction mixture is stirred at room temperature for 16 h. After completion, the reaction mixture is concentrated to get crude material which is purified to afford methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate (7). LCMS m/z 790.2 [M+1]⁺.

To a suspension of methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate (7, 1.0 eq) in tetrahydrofuran:methanol:water (3:2:1) (10 vol), lithium hydroxide monohydrate (4.0 eq) is added and the reaction mixture is stirred at room temperature for 4 h. After completion, the reaction mixture is concentrated to give crude material which is purified to afford methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate. LCMS m/z 680.2 [M+1]⁺.

To a solution of methyl (S)-4-(5-(3,6,9,12-tetraoxapentadec-14-yn-1-yl)-1,3,4-oxadiazol-2-yl)-2-(4-(N-((2-amino-4-hydroxypteridin-6-yl)methyl)-2,2,2-trifluoroacetamido)benzamido)butanoate (1.0 eq) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq) in dimethylsulfoxide (20 vol), tetrakis(acetonitrile)copper(I) hexafluorophosphate (2.8 eq) is added and the reaction mixture is stirred at room temperature for 1 h. After completion, was added the reaction mixture is quenched with acetic acid and purified to afford (S)-2-(4-(((2-amino-4-hydroxypteridin-6-yl)methyl)amino)benzamido)-4-(5-(1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-2,5,8,11-tetraoxatridecan-13-yl)-1,3,4-oxadiazol-2-yl)butanoic acid (Cpd. No. I-27). LCMS m/z 622.2 [M+2]⁺⁺.

### 6.1.18. Example 18: Compound I-28

### 4-(((2-amino-4-hydroxypteridin-6-yl)methyl)amino)-N—((S)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-18-(2H-tetrazol-5-yl)-2,5,8,11-tetraoxa-14-azaoctadecan-18-yl)benzamide (I-28)

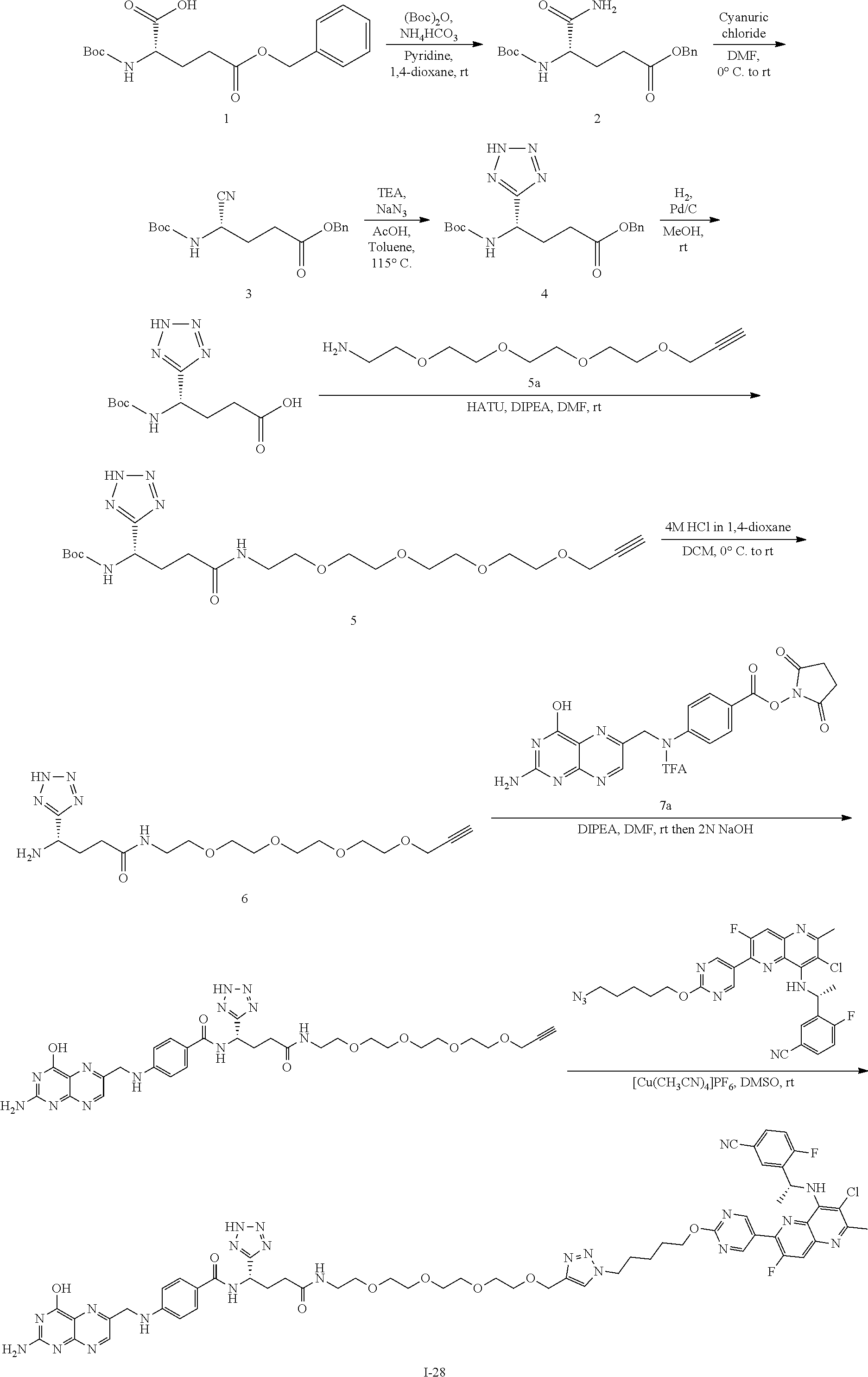

To a solution of (S)-5-(benzyloxy)-2-((tert-butoxycarbonyl)amino)-5-oxopentanoic acid (1, 1.0 eq, 1.0 g, 2.96 mmol) in 1,4-dioxane (20 mL), Boc anhydride (1.3 eq., 0.885 mL, 3.85 mmol), ammonium bicarbonate (1.3 eq., 0.305 g, 3.85 mmol) and pyridine (1.3 eq., 0.31 mL, 3.85 mmol) were added and the reaction mixture was stirred at room temperature for 16 h. After completion, the reaction mixture was concentrated, water was added and products extracted with ethyl acetate. The organic layer was dried over anhydrous sodium sulfate, filtered and concentrated to give a solid which was washed with hexane (2-3 times) and dried to afford benzyl (S)-5-amino-4-((tert-butoxycarbonyl)amino)-5-oxopentanoate (2) as a white solid. Yield: 1.05 g (crude), LCMS m/z 337.2 [M+1]⁺.

To a solution benzyl (S)-5-amino-4-((tert-butoxycarbonyl)amino)-5-oxopentanoate (2, 1.0 eq, 0.100 g, 0.297 mmol) in NN-dimethylformamide (1 mL) at 0° C. was added cyanuric chloride (0.65 eq, 0.035 g, 0.193 mmol) and the reaction mixture was stirred at room temperature for 16 h. After completion, water was added to the reaction mixture and products extracted with ethyl acetate. The organic layer was washed with water, dried over anhydrous sodium sulfate, filtered and concentrated to get crude material which was purified by column chromatography using silica gel (100-200 mesh) and 0-8% methanol in dichloromethane to afford benzyl (S)-4-((tert-butoxycarbonyl)amino)-4-cyanobutanoate (3) as a white solid. Yield: 0.075 g, 79.24%; LCMS m/z 319.2 [M+1]⁺.

To a solution of triethylamine (4.0 eq, 1.34 mL, 9.30 mmol) in toluene (10 mL) was added acetic acid (4.0 eq, 0.55 mL, 9.30 mmol) and the reaction mixture was stirred at room temperature for 5 minutes. Next, benzyl (S)-4-((tert-butoxycarbonyl)amino)-4-cyanobutanoate (3, 1.0 eq, 0.740 g, 2.32 mmol) and sodium azide (4.0 eq, 0.604 g, 9.30 mmol) were added and the reaction mixture was heated at 115° C. for 16 h. After completion, the reaction mixture was cooled and filtered and the filtrate was concentrated to give crude material which was purified by column chromatography using silica gel (100-200 mesh) and 0-10% methanol with 0.1% acetic acid in dichloromethane to afford benzyl (S)-4-((tert-butoxycarbonyl)amino)-4-(2H-tetrazol-5-yl)butanoate (4) as a white solid. Yield: 0.690 g, 81.44%; LCMS m/z 362.2 [M+1]⁺.

To a solution of benzyl (S)-4-((tert-butoxycarbonyl)amino)-4-(2H-tetrazol-5-yl)butanoate (4, 1.0 eq, 0.690 g, 1.91 mmol) in methanol (10 mL), 10% palladium on carbon (0.300 g) was added and the reaction mixture was stirred at room temperature under hydrogen gas atmosphere for 3 h. After completion, the reaction mixture was filtered through sintered glass funnel and the filtrate was concentrated, washed with diethyl ether and dried to afford (S)-4-((tert-butoxycarbonyl)amino)-4-(2H-tetrazol-5-yl)butanoic acid as a white solid. Yield: 0.500 g, 96.54%; LCMS m/z 272.0 [M+1]⁺.

To a solution of (S)-4-((tert-butoxycarbonyl)amino)-4-(2H-tetrazol-5-yl)butanoic acid (0.200 g, 0.737 mmol) in NN-dimethylformamide (2 mL) at 0° C. was added 3,6,9,12-tetraoxapentadec-14-yn-1-amine (5a, 1.0 eq, 0.171 g, 0.737 mmol), N,N-diisopropylethylamine (3.0 eq, 0.392 mL, 2.21 mmol) and 1-[bis(dimethylamino)methylene]-1H-1,2,3-triazolo[4,5-b]pyridinium 3-oxid hexafluorophosphate (HATU) (1.5 eq, 0.420 g, 1.11 mmol) and the reaction mixture was stirred at room temperature for 16 h. Water was added to the reaction mixture and products extracted with 10% methanol in dichloromethane. The organic layer was dried over anhydrous sodium sulfate, filtered and concentrated to give crude material which was purified by column chromatography using silica gel (100-200 mesh) and 0-15% methanol in dichloromethane to afford tert-butyl (S)-(17-oxo-20-(2H-tetrazol-5-yl)-4,7,10,13-tetraoxa-16-azaicos-1-yn-20-yl)carbamate (5) as a light brown viscous liquid. Yield: 0.080 g, 22.39%; LCMS m/z 485.2 [M+1]⁺.

A solution of tert-butyl N-[(1S)-3-[(3,6,9,12-tetraoxapentadec-14-yn-1-yl)carbamoyl]-1-(2H-1,2,3,4-tetrazol-5-yl)propyl]carbamate (5, 1.0 eq, 0.050 g, 0.103 mmol) in dichloromethane (0.5 mL) at 0° C. was treated with 4M hydrochloric acid in 1,4-dioxane (0.5 mL) and the reaction mixture was stirred at room temperature for 3 h. After completion, the reaction mixture was concentrated, azeotroped with dichloromethane (2-3 times), washed with diethyl ether (2-3 times) and dried to afford (S)-4-amino-N-(3,6,9,12-tetraoxapentadec-14-yn-1-yl)-4-(2H-tetrazol-5-yl)butanamide (6) as a light brown viscous liquid. Yield: 0.050 g (crude); LCMS m/z 385.2 [M+1]⁺.

To a solution of (S)-4-amino-N-(3,6,9,12-tetraoxapentadec-14-yn-1-yl)-4-(2H-tetrazol-5-yl)butanamide (6, 1.2 eq) and 2,5-dioxopyrrolidin-1-yl 4-(N-((2-amino-4-hydroxypteridin-6-yl)methyl)-2,2,2-trifluoroacetamido)benzoate (7a, 1.0 eq) in N,N-dimethylformamide (10 vol.), N,N-diisopropylethylamine (5.0 eq) is added and the reaction mixture is stirred at room temperature for 16 h. Next, 2M aqueous sodium hydroxide solution (3 vol.) is added and the reaction mixture is stirred at room temperature for 30 minutes then purified to afford (S)-4-(((2-amino-4-hydroxypteridin-6-yl)methyl)amino)-N-(17-oxo-20-(2H-tetrazol-5-yl)-4,7,10,13-tetraoxa-16-azaicos-1-yn-20-yl)benzamide. LCMS m/z 679.3 [M+1]⁺.

To a solution of (S)-4-(((2-amino-4-hydroxypteridin-6-yl)methyl)amino)-N-(17-oxo-20-(2H-tetrazol-5-yl)-4,7,10,13-tetraoxa-16-azaicos-1-yn-20-yl)benzamide (1.0 eq) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq) in dimethylsulfoxide (20 vol) is added tetrakis(acetonitrile)copper(I) hexafluorophosphate (2.8 eq) and the reaction mixture is stirred at room temperature for 1 h. After completion, the reaction mixture is quenched with acetic acid and purified to afford 4-(((2-amino-4-hydroxypteridin-6-yl)methyl)amino)-N—((S)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-18-(2H-tetrazol-5-yl)-2,5,8,11-tetraoxa-14-azaoctadecan-18-yl)benzamide (Cpd. No. I-28). LCMS m/z 621.7 [M+2]⁺⁺.

### 6.1.19. Example 19: Compound I-29

### (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)ethoxy)ethyl)-1H-1,2,3-triazol-4-yl)-15-oxo-2,5,8,11-tetraoxa-14-azanonadecan-19-oic acid (Compound I-29)

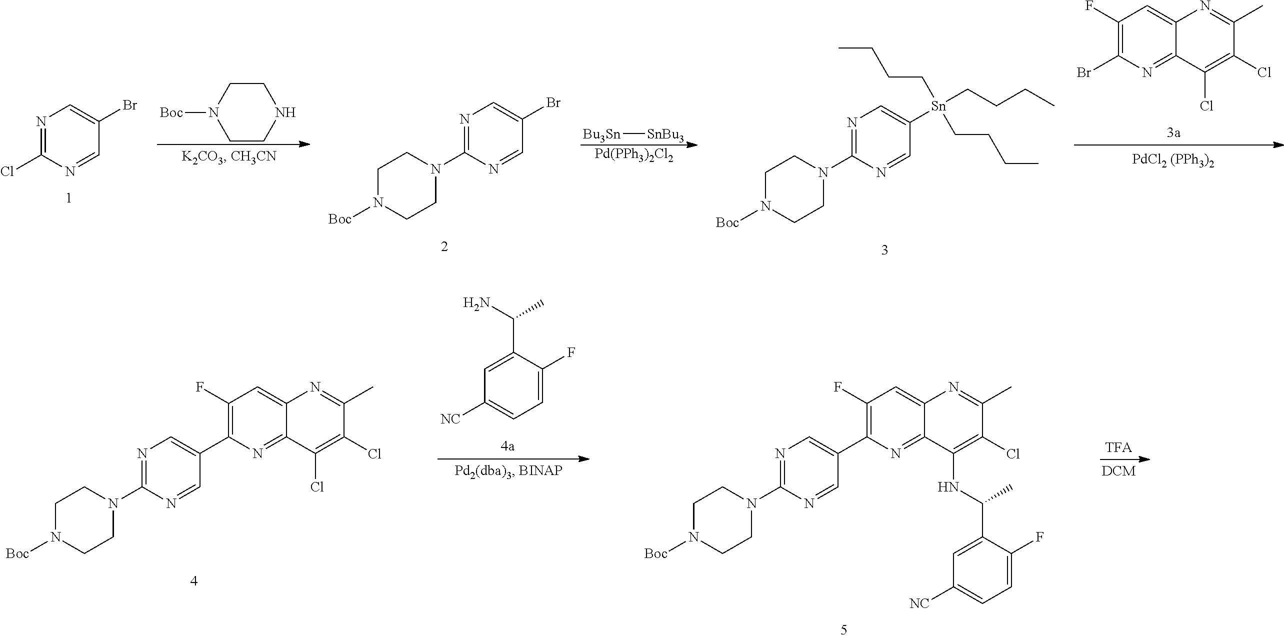

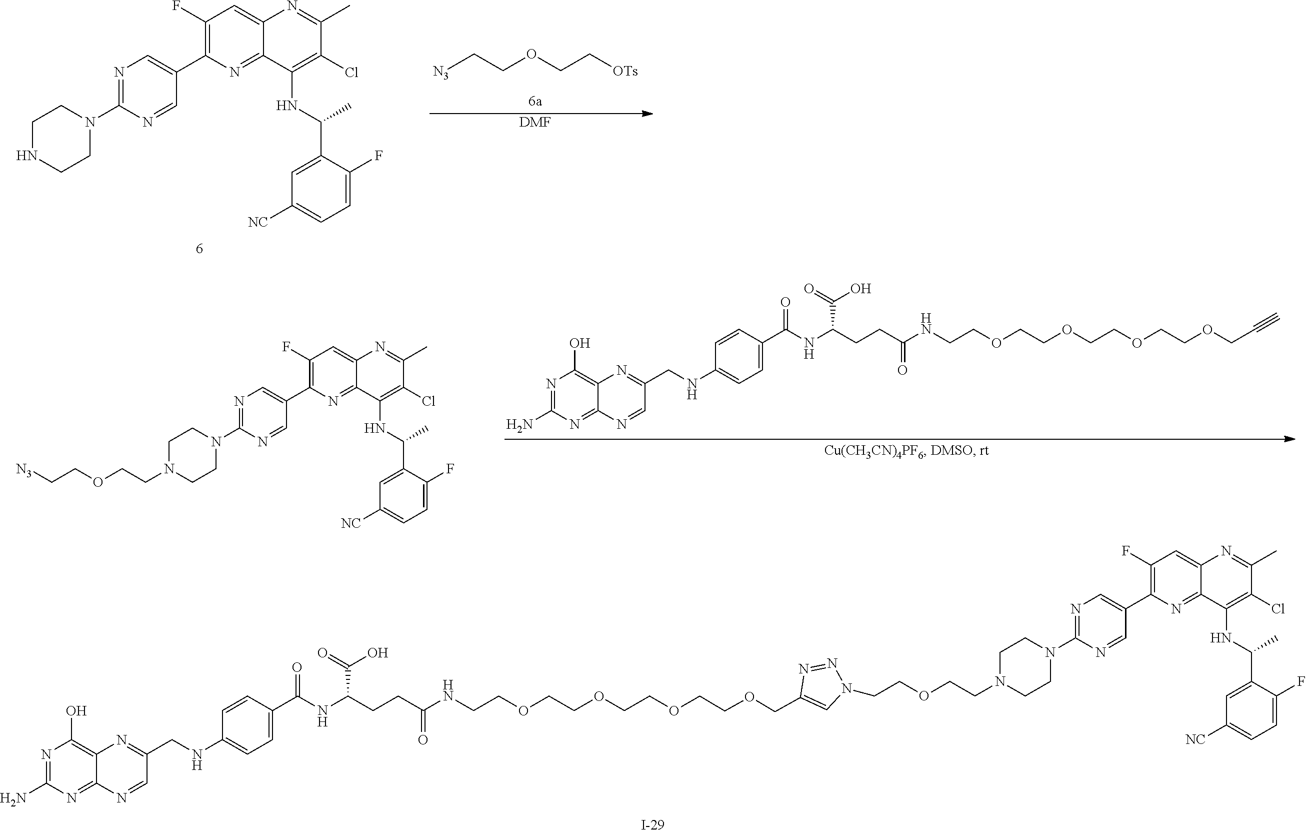

To a solution of 5-bromo-2-chloropyrimidine (1, 1.0 eq, 2.00 g, 10.3 mmol) and tert-butyl piperazine-1-carboxylate (1a, 1.0 eq, 1.93 g, 10.3 mmol) in acetonitrile (16 mL), potassium carbonate (2.0 eq, 2.86 g, 20.7 mmol) was added at room temperature, and the reaction mixture was heated at 110° C. for 16 h. After completion, the reaction mixture was concentrated under reduced pressure to give crude material which was purified by flash column chromatography over silica gel using 5-30% ethyl acetate in hexane as eluent to afford tert-butyl 4-(5-bromopyrimidin-2-yl)piperazine-1-carboxylate (2) as a white solid. Yield 2.60 g, 73.3%; LCMS m/z 243.0 [M−Boc+l]⁺.

A solution of tert-butyl 4-(5-bromopyrimidin-2-yl)piperazine-1-carboxylate (2, 1.0 eq, 3.20 g, 9.32 mmol) and bis(tributylstannane)(1.2 eq, 6.51 g, 11.2 mmol) in 1,4-dioxane (90 mL) was purged with nitrogen for 10 minutes then palladium (II) bis(triphenylphosphane)dichloride (0.05 eq, 0.327 g, 0.466 mmol) was added and reaction mixture was again purged with nitrogen. The reaction mixture was then heated at 100° C. for 16 h. After completion, the reaction mixture was filtered through a pad of celite and the filtrate was concentrated under reduced pressure to give crude material which was purified by flash column chromatography over silica gel using 5-30% ethyl acetate in hexane as eluent to afford tert-butyl 4-[5-(tributylstannyl)pyrimidin-2-yl]piperazine-1-carboxylate (3) as colorless viscous liquid. Yield 1.30 g, 25.1%; LCMS m/z 555.26 [M+1]⁺.

A solution of 6-bromo-3,4-dichloro-7-fluoro-2-methyl-1,5-naphthyridine (3a, 1.0 eq, 1.70 g, 5.48 mmol) and tert-butyl 4-[5-(tributylstannyl)pyrimidin-2-yl]piperazine-1-carboxylate (3, 1.1 eq, 3.34 g, 6.03 mmol) in 1,4-dioxane (20 mL) was purged with nitrogen gas for 10 minutes then, palladium(II) bis(triphenylphosphane)dichloride (0.05 eq 0.192 g, 0.27.4 mmol) was added and again purged with nitrogen for 10 minutes. The reaction mixture was stirred at 100° C. under a nitrogen atmosphere for 4 h. After completion, the reaction mixture was filtered through a pad of celite and the filtrate was concentrated under reduced pressure to give crude material which was purified by flash column chromatography over silica gel using 15-50% ethyl acetate in hexane as eluent to afford tert-butyl 4-(5-(7,8-dichloro-3-fluoro-6-methyl-1,5-naphthyridin-2-yl)pyrimidin-2-yl)piperazine-1-carboxylate (4) as off white solid. Yield 1.0 g, 37.0%; LCMS m/z 491.08 [M−1]⁻.

A solution of (R)-3-(1-aminoethyl)-4-fluorobenzonitrile hydrochloride (4a, 1.0 eq, 0.20 g, 1.22 mmol) and tert-butyl 4-(5-(7,8-dichloro-3-fluoro-6-methyl-1,5-naphthyridin-2-yl)pyrimidin-2-yl)piperazine-1-carboxylate (4, 1.0 eq, 0.60 g, 1.22 mmol), cesium carbonate (1.5 eq, 0.59 g, 1.83 mmol), tris(dibenzylideneacetone)-dipalladium(0) (0.11 g, 0.1 eq, 0.122 mmol), 2,2′-bis(diphenylphosphino)-1,1′-binaphthyl (0.20 eq, 0.15 g, 0.244 mmol) in anhydrous toluene (12 mL) and 1,4-dioxane (12 mL) was purged with nitrogen for 10 minutes. The reaction mixture was then stirred at 100° C. under a nitrogen atmosphere for 4 h. After the completion of reaction, reaction mixture was filtered through celite and washed with ethyl acetate, water was added, and extracted with ethyl acetate. The combined organic layer was dried over sodium sulfate, filtered, and concentrated under reduced pressure to get crude which was purified by flash chromatography over silica gel using 20 to 50% of ethyl acetate in hexanes as eluent to afford tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)piperazine-1-carboxylate (5) as an off white solid. Yield 0.40 g, 40.2%; LCMS m/z 621.33 [M+1]⁺.

To a stirred solution of tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)piperazine-1-carboxylate (5, 1.0 eq, 0.40 g, 0.644 mmol) in dichloromethane (8 mL) at 0° C., trifluoroacetic acid (2 mL) was added. The reaction mixture was then stirred at room temperature for 4 h. After completion, the reaction mixture was concentrated under reduced pressure to afford crude (R)-3-(1-((3-chloro-7-fluoro-2-methyl-6-(2-(piperazin-1-yl)pyrimidin-5-yl)-1,5-naphthyridin-4-yl)amino)ethyl)-4-fluorobenzonitrile (6) as a brown viscous liquid. Yield 0.40 g, LCMS m/z 521.28 [M+1]⁺.

A solution of (R)-3-(1-((3-chloro-7-fluoro-2-methyl-6-(2-(piperazin-1-yl)pyrimidin-5-yl)-1,5-naphthyridin-4-yl)amino)ethyl)-4-fluorobenzonitrile (6, 1.0 eq, 0.16 g, 0.307 mmol) and 2-(2-azidoethoxy)ethyl 4-methylbenzenesulfonate (1.1 eq, 0.096 g, 0.338 mmol) in N,N-dimethyl formamide (4.0 mL), was stirred at 60° C. for 16 h. After completion, the reaction mixture was diluted with ice water and extracted with ethyl acetate. The organic layer was dried over anhydrous sodium sulphate, filtered and concentrated under reduced pressure to give crude material which was purified by flash column chromatography over silica gel using 0-5% methanol in dichloromethane as eluent to afford (R)-3-(1-((6-(2-(4-(2-(2-azidoethoxy)ethyl)piperazin-1-yl)pyrimidin-5-yl)-3-chloro-7-fluoro-2-methyl-1,5-naphthyridin-4-yl)amino)ethyl)-4-fluorobenzonitrile. Yield 0.045 g, 23.1%; LCMS m/z 634.44 [M+1]⁺.

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq, 0.030 g, 0.045 mmol) and (R)-3-(1-((6-(2-(4-(2-(2-azidoethoxy)ethyl)piperazin-1-yl)pyrimidin-5-yl)-3-chloro-7-fluoro-2-methyl-1,5-naphthyridin-4-yl)amino)ethyl)-4-fluorobenzonitrile (1.2 eq, 0.045 g, 0.055 mmol) in dimethyl sulfoxide (1.5 mL), tetrakis(acetonitrile)copper(I) hexafluorophosphate (2.8 eq, 0.047 g, 0.128 mmol) was added and the reaction mixture was stirred at room temperature for 0.5 h. After completion, the reaction mixture was quenched with acetic acid (0.10 mL) and directly purified by prep HPLC (26-40% acetonitrile in water with 0.10% TFA). Fractions containing the desired product were combined and lyophilized to dryness to afford (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)ethoxy)ethyl)-1H-1,2,3-triazol-4-yl)-15-oxo-2,5,8,11-tetraoxa-14-azanonadecan-19-oic acid (Cpd. No. I-29) as a yellow solid. Yield: 0.025 g, 42.3%; LCMS m/z 645.05 [M+2]⁺⁺; ¹H NMR (400 MHz, DMSO-d₆ with D₂O) δ8.83 (s, 2H), 8.63 (s, 1H), 8.09 (s, 1H), 8.05 (d, J=10.8 Hz, 1H), 7.95 (d, J=6.0 Hz, 1H), 7.77 (bs, 1H), 7.59 (d, J=8.4 Hz, 2H), 7.31 (t, J=9.6 Hz, 1H), 6.60 (d, J=8.0 Hz, 2H), 6.50 (d, J=6.4 Hz, 1H), 4.75 (bs, 2H), 4.57 (bs, 2H), 4.50 (s, 2H), 4.46 (s, 2H), 4.25-4.20 (m, 1H), 3.87 (s, 2H), 3.54 (s, 2H), 3.48 (s, 3H), 3.40 (d, J=6.8 Hz, 10H), 3.30 (t, J=4.8 Hz, 5H), 3.13-3.08 (m, 2H), 2.67-2.66 (m, 3H), 2.15 (t, J=6.8 Hz, 2H), 2.02-1.99 (m, 1H), 1.90-1.87 (m, 1H), 1.67 (d, J=6.4 Hz, 3H).

### 6.1.20. Example 20: Compound I-30

### (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)-4-hydroxypiperidin-1-yl)ethoxy)ethyl)-1H-1,2,3-triazol-4-yl)-15-oxo-2,5,8,11-tetraoxa-14-azanonadecan-19-oic acid (Compound I-30)

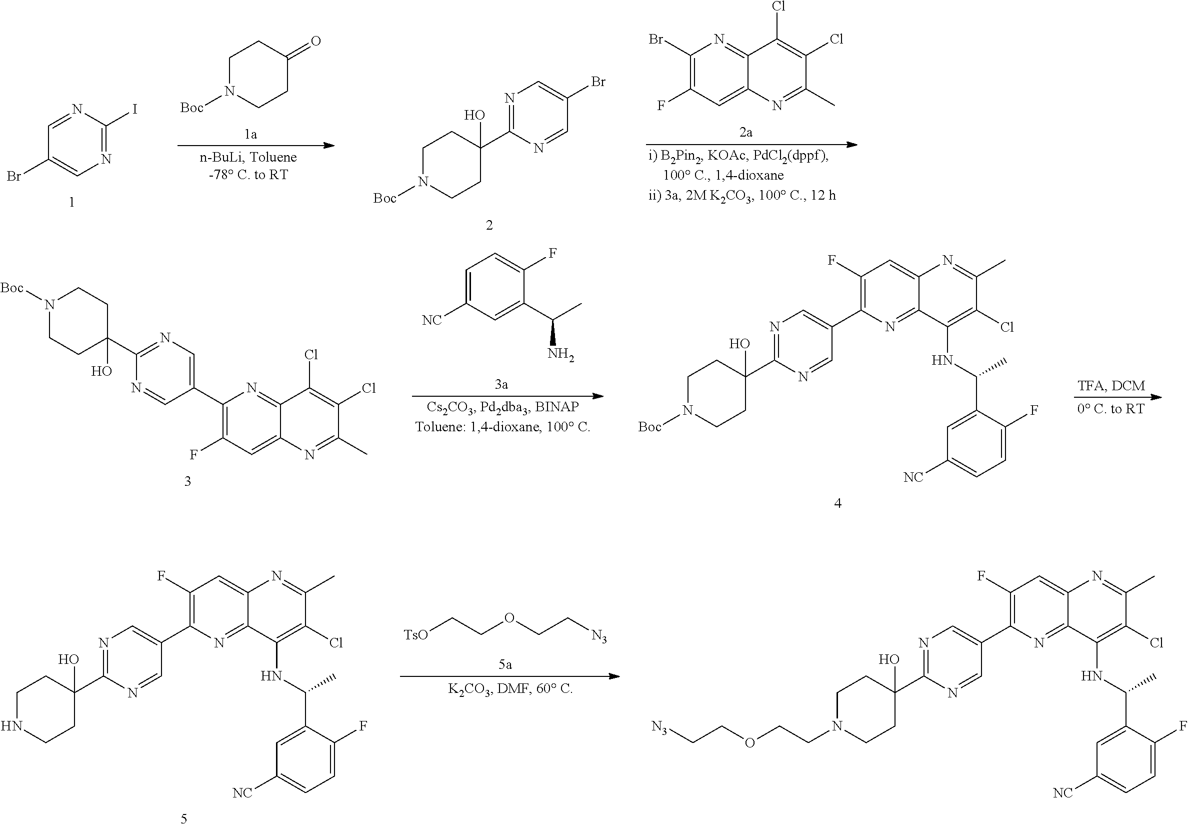

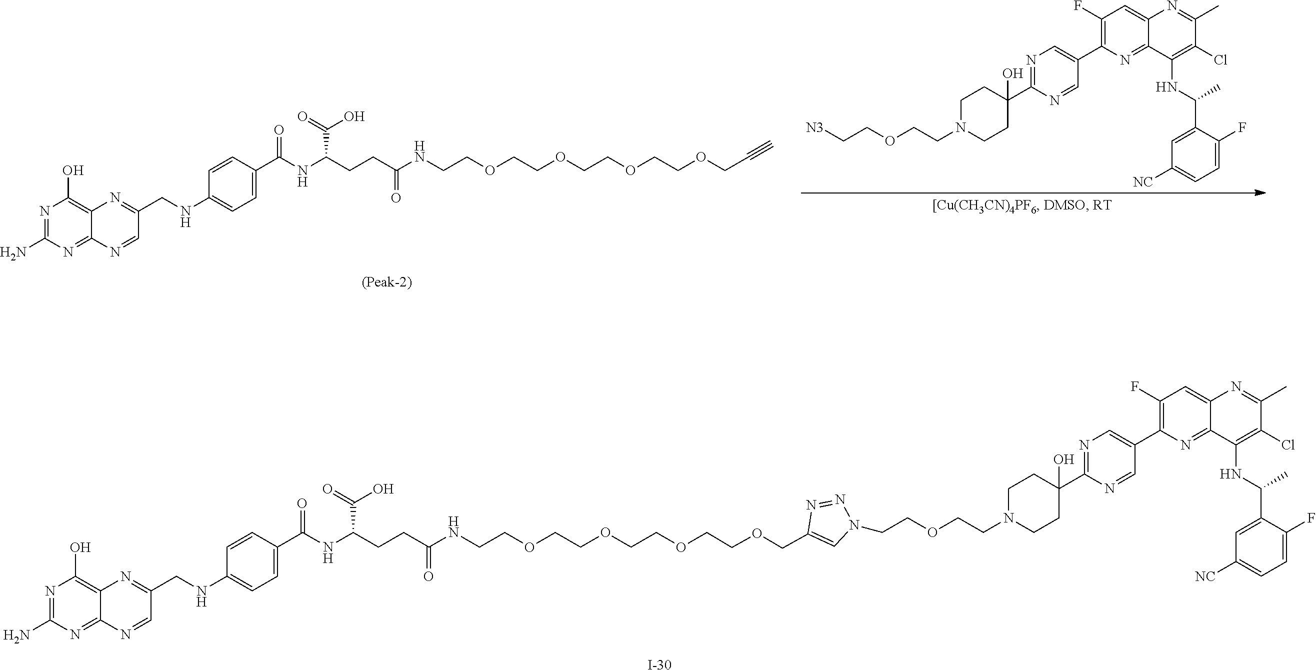

A solution of 5-bromo-2-iodopyrimidine (1, 1.0 eq, 5.0 g, 17.6 mmol) in toluene (100 mL) was cooled at −78° C., n-butyl lithium (2.5 M in Hexane, 1.1 eq, 7.72 mL, 19.3 mmol) was added and the reaction mixture was stirred at −78° C. for 30 minutes. A solution of tert-butyl 4-oxopiperndine-1-carboxylate (1a, 1.1 eq, 3.85 g, 19.3 mmol) in toluene (25 mL) was added and the reaction mixture was stirred at room temperature for 16 h. After completion, the reaction mixture was again cooled at −78° C., quenched with saturated aqueous ammonium chloride and extracted with ethyl acetate. The organic layer was dried over anhydrous sodium sulfate, filtered and concentrated to give crude material which was purified by column chromatography using silica gel (100-200 mesh) and 0-20% ethyl acetate in hexane to afford tert-butyl 4-(5-bromopyrimidin-2-yl)-4-hydroxypiperidine-1-carboxylate (2) as a light yellow viscous liquid. Yield: 1.8 g, 23.5%; LC-MS m/z 358.13 [M+1]⁺.

A mixture of tert-butyl 4-(5-bromopyrimidin-2-yl)-4-hydroxypiperidine-1-carboxylate (2, 1.2 eq, 1.59 g, 4.45 mmol), 4,4,5,5-tetramethyl-2-(4,4,5,5-tetramethyl-1,3,2-dioxaborolan-2-yl)-1,3,2-dioxaborolane (1.5 eq, 1.15 g, 3.71 mmol) and potassium acetate (1.8 eq, 0.655 g, 6.68 mmol) in 1,4-dioxane (20.0 mL) was purged with nitrogen for 5 minutes. [1,1′-Bis(diphenylphosphino)ferrocene]palladium(II) dichloride dichloromethane complex (0.125 eq, 0.378 g, 0.464 mmol) was then added and the reaction mixture was again purged with nitrogen for 5 minutes. The reaction mixture was heated at 80° C. for 3 h. After cooling to room temperature, 6-bromo-3,4-dichloro-7-fluoro-2-methyl-1,5-naphthyridine (2a, 1.0 eq, 1.15 g, 3.71 mmol) and potassium carbonate (2M aqueous solution) (2.5 eq, 4.64 mL, 9.28 mmol) were added to the reaction. The reaction mixture was purged with nitrogen and again heated at 100° C. for 12 h. After completion, the reaction mixture was diluted with ethyl acetate and water. The organic layer was separated, dried over sodium sulfate, filtered and concentrated under reduced pressure to obtain crude material which was purified by flash chromatography (silica mesh: 100-200; elution: 15-20% ethyl acetate in hexane) to afford tert-butyl 4-(5-(7,8-dichloro-3-fluoro-6-methyl-1,5-naphthyridin-2-yl)pyrimidin-2-yl)-4-hydroxypiperidine-1-carboxylate (3) as light pink solid. Yield: 0.420 g, 22.3%; LC-MS m/z 508.12 [M+1]⁺.

A solution of tert-butyl 4-(5-(7,8-dichloro-3-fluoro-6-methyl-1,5-naphthyridin-2-yl)pyrimidin-2-yl)-4-hydroxypiperidine-1-carboxylate (3, 1.0 eq, 0.49 g, 0.964 mmol), 3-[(1R)-1-aminoethyl]-4-fluorobenzonitrile hydrochloride (3a, 1.2 eq, 0.190 g, 1.16 mmol) and cesium carbonate (1.5 eq, 0.471 g, 1.45 mmol) in 1,4-dioxane:toluene (1:1) (5.0 mL: 5.0 mL) was purged with nitrogen for 5 minutes. [2′-(diphenylphosphanyl)-[1,1′-binaphthalen]-2-yl]diphenylphosphane (0.2 eq, 0.12 g, 0.193 mmol) and tris((1E,4E)-1,5-diphenylpenta-1,4-dien-3-one) palladium (0.1 eq, 0.078 g, 0.096 mmol) were added and the reaction mixture was again purged with nitrogen for 5 minutes. The reaction mixture was heated at 100° C. for 12 h. After completion, the reaction mixture was diluted with ethyl acetate and water. The organic layer was separated, dried over sodium sulfate, filtered, and concentrated under reduced pressure to obtain crude. The crude was purified by flash chromatography (silica mesh: 100-200:elution: 15 to 30% ethyl acetate in hexanes) to afford tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)-4-hydroxypiperidine-1-carboxylate (4) as yellow solid. Yield: 0.35 g, 57%: LC-MS m/z 637.1 [M+1]⁺.

To a solution of tert-butyl (R)-4-(5-(7-chloro-8-((1-(5-cyano-2-fluorophenyl)ethyl)amino)-3-fluoro-6-methyl-1,5-naphthyridin-2-yl)pyrimidin-2-yl)-4-hydroxypiperidine-1-carboxylate (4, 1.0 eq, 0.1 g, 0.157 mmol) in dichloromethane (0.5 mL) was added 2,2,2-trifluoroacetic acid (0.5 mL) at 0° C. The reaction mixture was stirred at room temperature for 4 h. After that, the reaction mixture was concentrated under reduced pressure, azeotroped with dichloromethane (2 or 3 times) and dried to afford (R)-3-(1-((3-chloro-7-fluoro-6-(2-(4-hydroxypiperidin-4-yl)pyrimidin-5-yl)-2-methyl-1,5-naphthyridin-4-yl)amino)ethyl)-4-fluorobenzonitrile (5) as yellow viscous liquid. Yield: 0.15 g (crude); LC-MS m/z 537.1 [M+1]⁺.

To a solution of (R)-3-(1-((3-chloro-7-fluoro-6-(2-(4-hydroxypiperidin-4-yl)pyrimidin-5-yl)-2-methyl-1,5-naphthyridin-4-yl)amino)ethyl)-4-fluorobenzonitrile (5, 1.0 eq, 0.15 g, 0.280 mmol) in N,N dimethylformamide (1.5 mL) was added potassium carbonate (3.0 eq, 0.116 g, 0.840 mmol). A solution of 2-(2-azidoethoxy)ethyl 4-methylbenzenesulfonate (1.1 eq, 0.087 g, 0.308 mmol) in N,N-dimethylformamide (0.5 mL) was added and the reaction mixture was heated at 60° C. for 5 h. After completion, the reaction mixture was diluted with water and extracted with ethyl acetate. The organic layer was dried over sodium sulfate, filtered and concentrated under reduced pressure to obtain crude. The crude was purified by flash chromatography (silica mesh: 100-200; elution: 2-3% methanol in dichloromethane) to afford (R)-3-(1-((6-(2-(1-(2-(2-azidoethoxy)ethyl)-4-hydroxypiperidin-4-yl)pyrimidin-5-yl)-3-chloro-7-fluoro-2-methyl-1,5-naphthyridin-4-yl)amino)ethyl)-4-fluorobenzonitrile as yellow liquid. Yield: 0.06 g, 33%; LCMS: 649.23 [M+1]+.

To a solution of (S)-20-(4-(((2-amino-4-hydroxypteridin-6-yl)methyl)amino)benzamido)-17-oxo-4,7,10,13-tetraoxa-16-azahenicos-1-yn-21-oic acid (1.0 eq) and (R)-3-(1-((6-(2-(1-(2-(2-azidoethoxy)ethyl)-4-hydroxypiperidin-4-yl)pyrimidin-5-yl)-3-chloro-7-fluoro-2-methyl-1,5-naphthyridin-4-yl)amino)ethyl)-4-fluorobenzonitrile (1.0 eq) in dimethylsulfoxide (20 vol), tetrakis(acetonitrile)copper(I) hexafluorophosphate (2.8 eq) is added and the reaction mixture is stirred at room temperature for 1 h. After completion, the reaction mixture is quenched with acetic acid and purified to afford (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)-4-hydroxypiperidin-1-yl)ethoxy)ethyl)-1H-1,2,3-triazol-4-yl)-15-oxo-2,5,8,11-tetraoxa-14-azanonadecan-19-oic acid (Cpd. No. I-30).

### 6.1.21. Example 21: Compound I-31

### (S)-1-(1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-18-(5-(methyl((2-methyl-4-oxo-1,4-dihydroquinazolin-6-yl)methyl)amino)thiophene-2-carboxamido)-15-oxo-2,5,8,11-tetraoxa-14-azanonadecan-19-oic acid (Compound I-31)

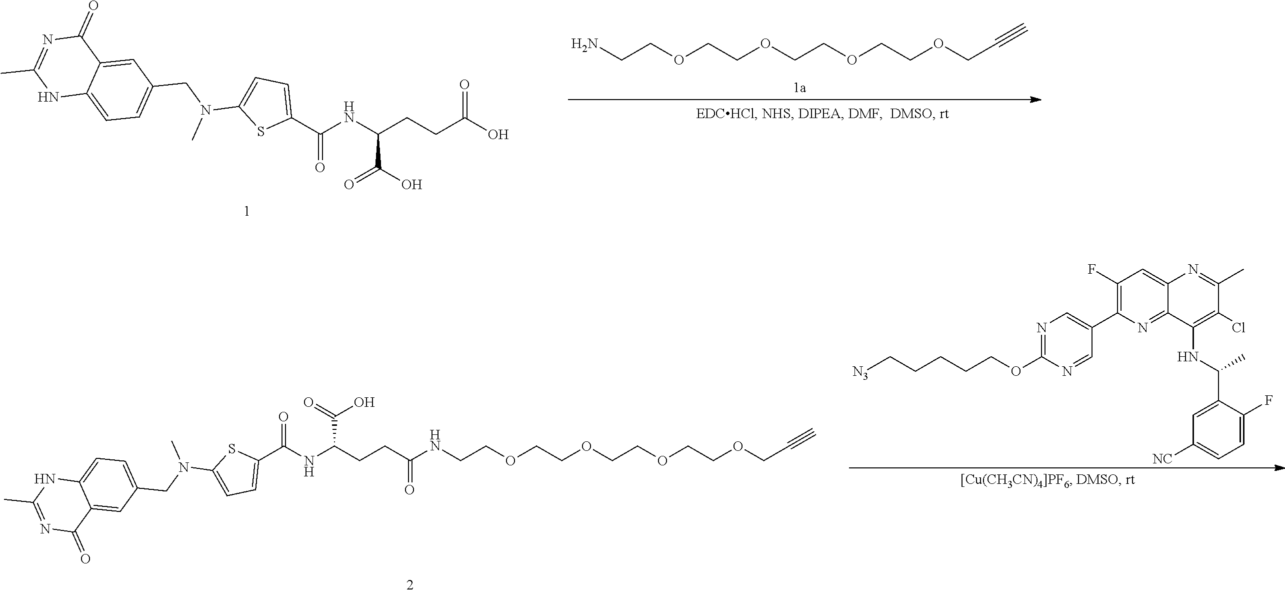 [SMILES: Cc1nc(=O)c2cc(CN(C)c3ccc(C(=O)N[C@@H](CCC(=O)O)C(=O)O)s3)ccc2[nH]1 ; C#CCOCCOCCOCCOCCN ; C#CCOCCOCCOCCOCCNC(=O)CC[C@H](NC(=O)c1ccc(N(C)Cc2ccc3[nH]c(C)nc(=O)c3c2)s1)C(=O)O ; Cc1nc2cc(F)c(-c3cnc(OCCCCCN=[N+]=[N-])nc3)nc2c(N[C@H](C)c2cc(C#N)ccc2F)c1Cl]

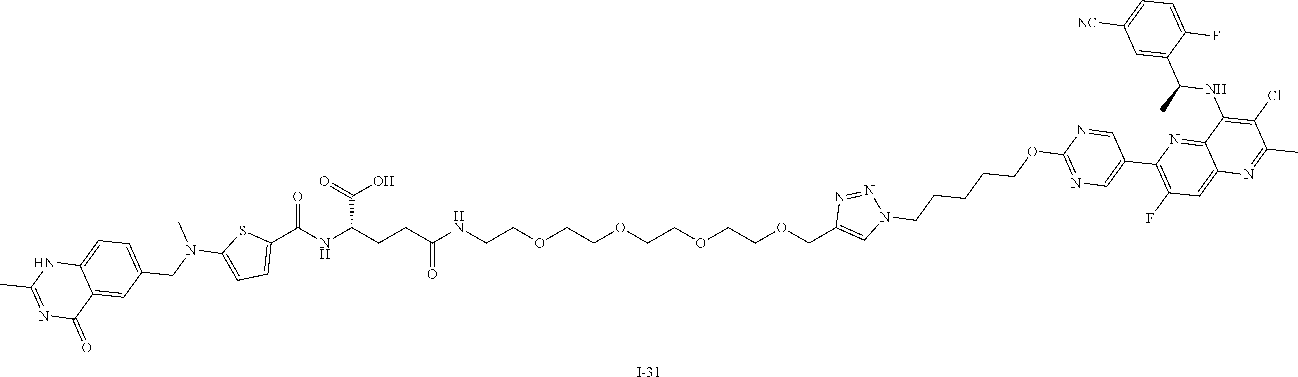

To a solution of (5-(methyl((2-methyl-4-oxo-1,4-dihydroquinazolin-6-yl)methyl)amino)thiophene-2-carbonyl)-L-glutamic acid (Raltitrexed) (1, 1.0 eq, 0.400 g, 0.872 mmol) in NN-dimethylformamide (8 mL) and dimethyl sulfoxide (8 mL), N-hydroxysuccinimide (1.1 eq, 0.110 g, 0.960 mmol), 1-(3-Dimethylaminopropyl)-3-ethylcarbodiimide hydrochloride (EDC·HCl) (1.1 eq, 0.184 g, 0.960 mmol) and NN-diisopropylethylamine (3.0 eq, 0.48 mL, 2.62 mmol) were added and the reaction mixture was stirred at room temperature for 1 h. Then, 3,6,9,12-tetraoxapentadec-14-yn-1-amine (1a, 1.0 eq, 0.202 g, 0.872 mmol) was added and the reaction mixture was stirred at room temperature for 16 h. After completion, the reaction mixture was directly purified by prep HPLC (24-47% acetonitrile in water with 0.1% acetic acid). Fractions containing the desired product were combined and lyophilized to dryness to afford (S)-20-(5-(methyl((2-methyl-4-oxo-1,4-dihydroquinazolin-6-yl)methyl)amino)thiophene-2-carboxamido)-17-oxo-4,7,10,13-tetraoxa-16-azahenicos-1-yn-21-oic acid (2) as a light yellow sticky solid. Yield: 0.050 g, 8.53%; LCMS m/z 672.57 [M+1]⁺.

To a solution of (S)-20-(5-(methyl((2-methyl-4-oxo-1,4-dihydroquinazolin-6-yl)methyl)amino)thiophene-2-carboxamido)-17-oxo-4,7,10,13-tetraoxa-16-azahenicos-1-yn-21-oic acid (2, 1.0 eq, 0.048 g, 0.071 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.040 g, 0.071 mmol) in dimethylsulfoxide (1.0 mL), tetrakis(acetonitrile)copper(I) hexafluorophosphate (2.8 eq, 0.074 g, 0.200 mmol) was added and reaction mixture was stirred at room temperature for 1 h. After completion, the reaction mixture was quenched with acetic acid (0.2 mL) and purified by prep HPLC (48-60% acetonitrile in water with 0.10% acetic acid). Fractions containing the desired product were combined and lyophilized to dryness to afford (S)-1-(1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-18-(5-(methyl((2-methyl-4-oxo-1,4-dihydroquinazolin-6-yl)methyl)amino)thiophene-2-carboxamido)-15-oxo-2,5,8,11-tetraoxa-14-azanonadecan-19-oic acid (Cpd. No. I-31) as an off white solid. Yield: 0.015 g, 17%; LCMS m/z 618.55 [M+2]⁺⁺; ¹H NMR (400 MHz, DMSO-d₆ with D₂O) δ8.93 (s, 2H), 8.01 (d, J=11.2 Hz, 2H), 7.90-7.85 (m, 2H), 7.72-7.68 (m, 1H), 7.61 (d, J=8.0 Hz, 1H), 7.51-7.47 (m, 2H), 7.22 (t, J=8.8 Hz, 1H), 6.30 (d, J=8.0 Hz, 1H), 5.91 (d, J=4.0 Hz, 1H), 4.57 (s, 2H), 4.46 (s, 2H), 4.35 (bs, 4H), 4.18-4.17 (m, 1H), 4.10-4.08 (m, 1H), 3.48-3.41 (m, 14H), 3.33-3.31 (m, 2H), 3.13 (d, J=6.0 Hz, 2H), 2.99 (s, 3H), 2.59 (s, 3H), 2.29 (s, 3H), 2.14-2.13 (m, 2H), 2.05-1.95 (m, 2H), 1.90-1.79 (m, 4H), 1.80-1.70 (m, 2H), 1.61 (d, J=6.8 Hz, 3H), 1.40-1.24 (m, 2H).

### 6.1.22. Example 22: Compound I-32

### (S)-18-(4-(2-(2-amino-4-oxo-4,7-dihydro-1H-pyrrolo[2,3-d]pyrimidin-5-yl)ethyl)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid

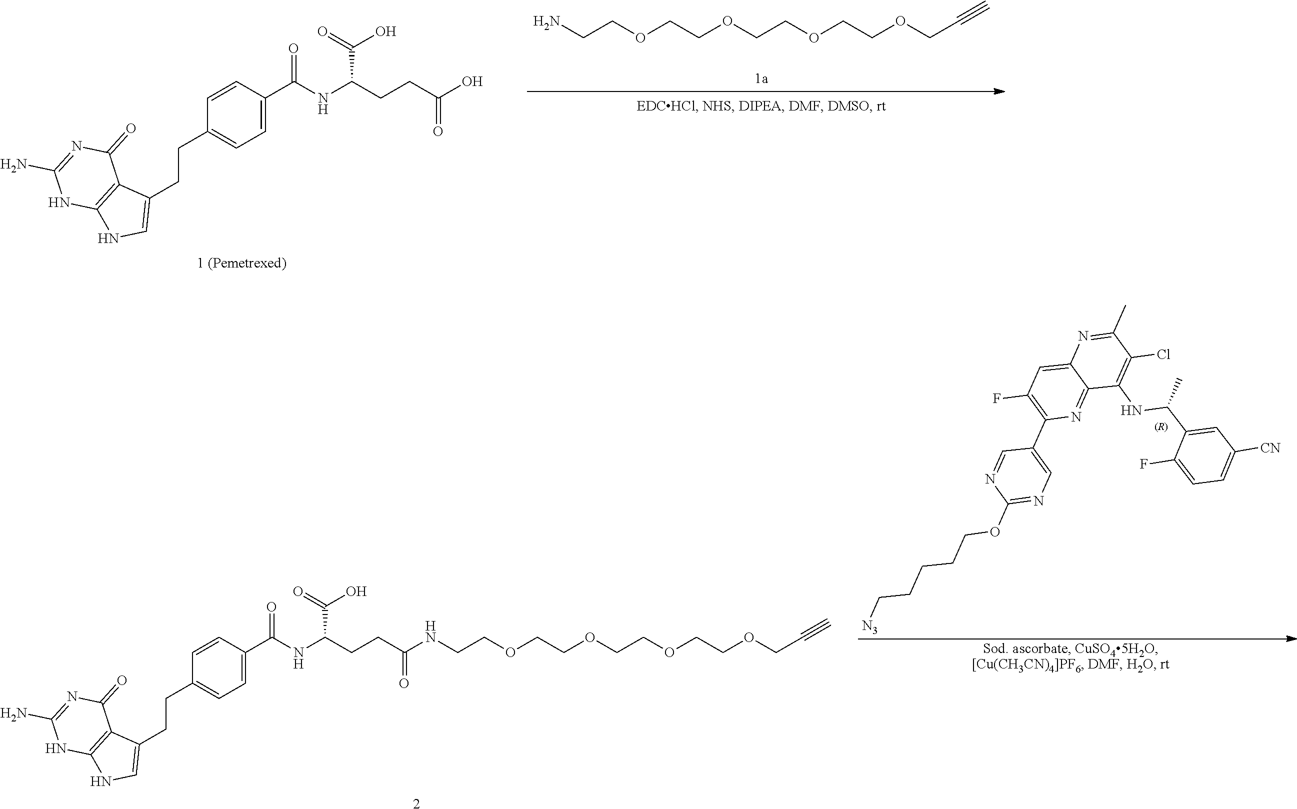

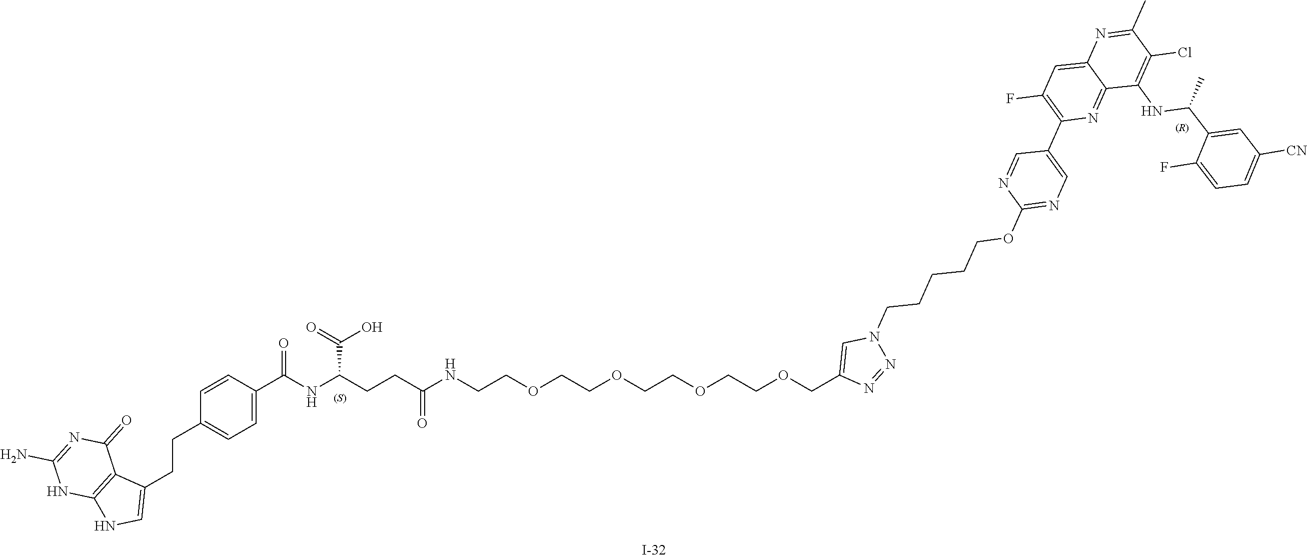

To a solution of (4-(2-(2-amino-4-oxo-4,7-dihydro-1H-pyrrolo[2,3-d]pyrimidin-5-yl)ethyl)benzoyl)-L-glutamic acid (Pemetrexed) (1, 1.0 eq, 0.500 g, 1.17 mmol) in N,N-dimethylformamide (10 mL) and dimethyl sulfoxide (10 mL), N-hydroxysuccinimide (1.1 eq, 0.148 g, 1.29 mmol), 1-(3-Dimethylaminopropyl)-3-ethylcarbodiimide hydrochloride (EDC·HCl) (1.1 eq, 0.247 g, 1.29 mmol) and N,N-diisopropylethylamine (3.0 eq, 0.64 mL, 3.51 mmol) were added and the reaction mixture was stirred at room temperature for 30 minutes. Then, 3,6,9,12-tetraoxapentadec-14-yn-1-amine (1a, 1.1 eq, 0.298 g, 1.29 mmol) was added and the reaction mixture was stirred at room temperature for 16 h. After completion, the reaction mixture was directly purified by prep HPLC (20-55% acetonitrile in water with 0.1% acetic acid). Fractions containing the desired product were combined and lyophilized to dryness to afford (S)-20-(4-(2-(2-amino-4-oxo-4,7-dihydro-1H-pyrrolo[2,3-d]pyrimidin-5-yl)ethyl)benzamido)-17-oxo-4,7,10,13-tetraoxa-16-azahenicos-1-yn-21-oic acid (2) as a yellow sticky solid. Yield: 0.100 g, 12.6%; LCMS m/z 641.2 [M+1]⁺.

To a solution of (S)-20-(4-(2-(2-amino-4-oxo-4,7-dihydro-1H-pyrrolo[2,3-d]pyrimidin-5-yl)ethyl)benzamido)-17-oxo-4,7,10,13-tetraoxa-16-azahenicos-1-yn-21-oic acid (2, 1.0 eq, 0.070 g, 0.109 mmol) and (R)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.061 g, 0.109 mmol) in N,N-dimethylformamide (1.8 mL) and water (0.2 mL), copper sulfate pentahydrate (0.05 eq, 0.001 g, 0.005 mmol) and sodium ascorbate (0.5 eq, 0.010 g, 0.054 mmol) were added and the reaction mixture was stirred at room temperature for 16 h. Then, tetrakis(acetonitrile)copper(I) hexafluorophosphate (1.0 eq, 0.040 g, 0.109 mmol) was added, and the reaction mixture was stirred at room temperature for another 1 h. After completion, the reaction mixture was quenched with acetic acid (0.2 mL) and directly purified by prep HPLC (33-65% acetonitrile in water with 0.1% formic acid). Fractions containing the desired compound were combined and lyophilized to afford (S)-18-(4-(2-(2-amino-4-oxo-4,7-dihydro-1H-pyrrolo[2,3-d]pyrimidin-5-yl)ethyl)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-15-oxo-2,5,8,11-tetraoxa-14-azanonadecan-19-oic acid (Cpd. No. I-32) as a light green solid. Yield: 0.015 g, 11.17%; LCMS m/z 602.76 [M+2]⁺; H NMR (400 MHz, DMSO-d₆) δ12.54 (bs, 1H), 10.60 (s, 1H), 10.14 (s, 1H), 9.04 (s, 2H), 8.56 (d, J=7.6 Hz, 1H), 8.17 (d, J=11.6 Hz, 1H), 8.10 (s, 1H), 8.01 (d, J=5.2 Hz, 1H), 7.91 (t, J=5.6 Hz, 1H), 7.77 (d, J=8.0 Hz, 3H), 7.33-7.26 (m, 3H), 6.98-6.96 (m, 1H), 6.35-6.29 (m, 2H), 6.00 (s, 2H), 4.50 (s, 2H), 4.42-4.36 (m, 4H), 4.35-4.29 (m, 1H), 3.52-3.46 (m, 14H), 3.19-3.16 (m, 2H), 2.98-2.94 (m, 2H), 2.85-2.82 (m, 2H), 2.63 (s, 3H), 2.24-2.20 (m, 2H), 2.10-2.06 (m, 1H), 1.94-1.88 (m, 3H), 1.83 (t, J=7.2 Hz, 2H), 1.65 (d, J=6.8 Hz, 3H), 1.40 (t, J=7.2 Hz, 2H).

### 6.1.23. Example 23: Compound I-33

### N5-(2-(2-((1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)-N2-(4-(((2,4-diaminopteridin-6-yl)methyl)(methyl)amino)benzoyl)-L-glutamine (Compound I-33)

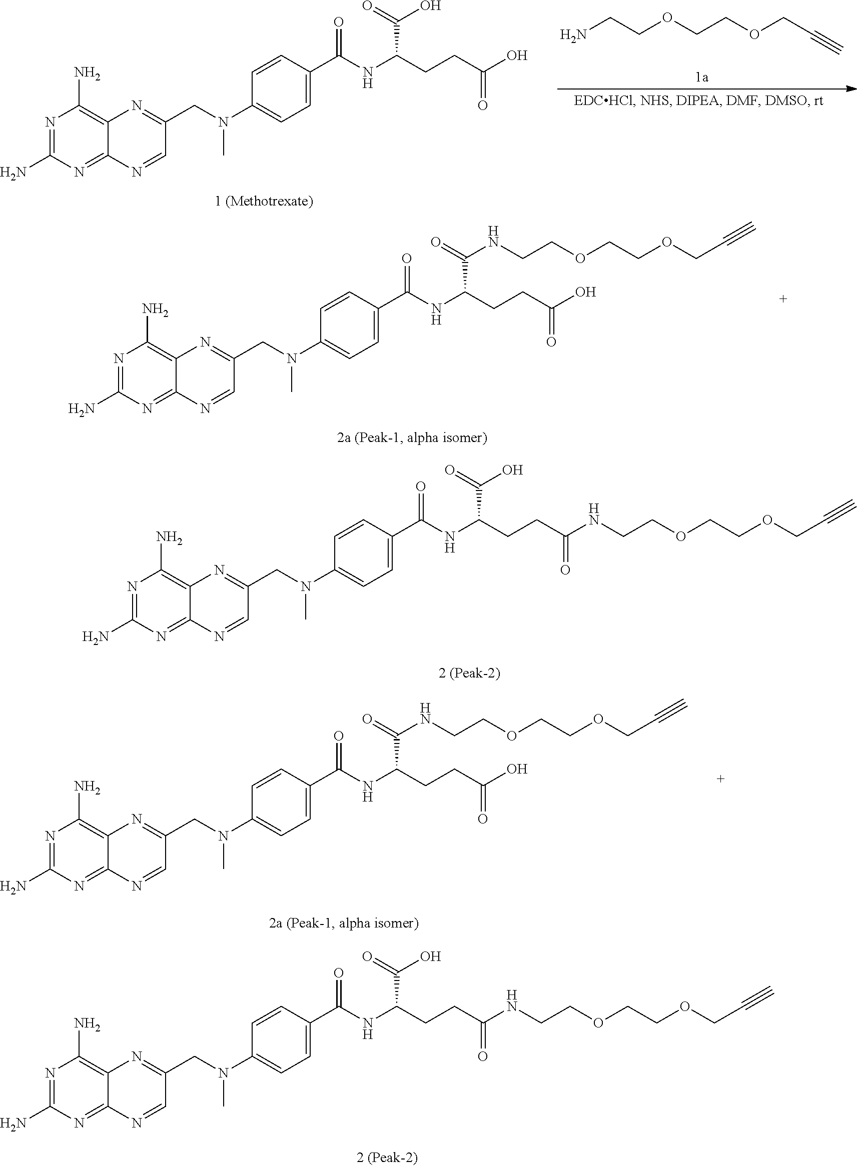

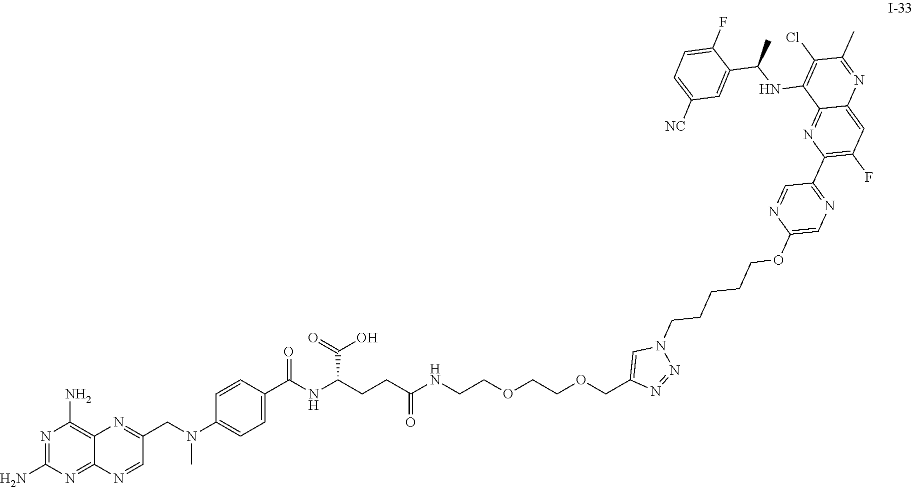 [SMILES: Cc1nc2cc(F)c(-c3cnc(OCCCCCn4cc(COCCOCCNC(=O)CC[C@H](NC(=O)c5ccc(N(C)Cc6cnc7nc(N)nc(N)c7n6)cc5)C(=O)O)nn4)cn3)nc2c(N[C@H](C)c2cc(C#N)ccc2F)c1Cl]

To a solution of (4-(((2,4-diaminopteridin-6-yl)methyl)(methyl)amino)benzoyl)-L-glutamic acid (Methotrexate) (1, 1.0 eq, 0.900 g, 1.98 mmol) in NN-dimethylformamide (18 mL) and dimethyl sulfoxide (18 mL), N-hydroxysuccinimide (1.1 eq, 0.251 g, 2.18 mmol), 1-(3-dimethylaminopropyl)-3-ethylcarbodiimide hydrochloride (EDC·HCl) (1.1 eq, 0.418 g, 2.18 mmol) and NN-diisopropylethylamine (3.0 eq, 1.1 mL, 5.94 mmol) were added and the reaction mixture was stirred at room temperature for 1 h. Then, 2-(2-(prop-2-yn-1-yloxy)ethoxy)ethan-1-amine (1a, 1.1 eq, 0.312 g, 2.18 mmol) was added and the reaction mixture was stirred at same temperature for 16 h. After completion, the reaction mixture was directly purified by prep HPLC (10-21% acetonitrile in water with 0.1% acetic acid). The faster eluting fractions were combined and lyophilized to dryness to afford (S)-4-(4-(((2,4-diaminopteridin-6-yl)methyl)(methyl)amino)benzamido)-5-oxo-5-((2-(2-(prop-2-yn-1 yloxy)ethoxy)ethyl)amino)pentanoic acid (2a, Peak-1, alpha isomer) as a yellow solid. Yield: 0.110 g, 9.6%; LCMS m/z 580.55 [M+1]. The slower eluting fractions were combined and lyophilized to dryness to afford N²-(4-(((2,4-diaminopteridin-6-yl)methyl)(methyl)amino)benzoyl)-N5-(2-(2-(prop-2-yn-1-yloxy)ethoxy)ethyl)-L-glutamine (2, Peak-2) as a yellow solid. Yield: 0.180 g, 15.7%; LCMS m/z 580.64 [M+1]⁺.

To a solution of N²-(4-(((2,4-diaminopteridin-6-yl)methyl)(methyl)amino)benzoyl)-N⁵-(2-(2-(prop-2-yn-1-yloxy)ethoxy)ethyl)-L-glutamine (2, 1.0 eq, 0.050 g, 0.086 mmol) and (S)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.048 g, 0.086 mmol) in dimethyl sulfoxide (1.5 mL), tetrakis(acetonitrile)copper(I) hexafluorophosphate (2.8 eq, 0.090 g, 0.242 mmol) was added and the reaction mixture was stirred at room temperature for 1 h. After completion, the reaction mixture was quenched with acetic acid (0.2 mL) and directly purified by prep HPLC (32-50% acetonitrile in water with 0.1% acetic acid). Fractions containing the desired product were combined and lyophilized to dryness to afford N⁵-(2-(2-((1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)-N²-(4-(((2,4-diaminopteridin-6-yl)methyl)(methyl)amino)benzoyl)-L-glutamine (Cpd. No. I-33) as a yellow solid. Yield: 0.008 g, 8.1%; LCMS m/z 572.71 [M+2]⁺⁺; ¹H NMR (400 MHz, DMSO-d₆ with D₂O) δ8.99 (s, 2H), 8.56 (bs, 1H), 8.09-8.06 (m, 2H), 7.94 (d, J=4.8 Hz, 1H), 7.72-7.67 (m, 3H), 7.26 (t, J=8.4 Hz, 1H), 6.78 (d, J=8.8 Hz, 2H), 6.30 (d, J=6.4 Hz, 1H), 4.77 (s, 2H), 4.47 (s, 2H), 4.36 (d, J=6.4 Hz, 4H), 4.25 (bs, 1H), 3.48-3.45 (m, 6H), 3.33-3.30 (m, 2H), 3.17 (s, 3H), 3.16-3.12 (m, 3H), 2.61 (s, 3H), 2.18-2.15 (m, 2H), 2.06-1.99 (m, 2H), 1.88-1.84 (m, 2H), 1.78-1.76 (m, 2H), 1.62 (d, J=6.8 Hz, 3H), 1.36-1.34 (m, 2H).

### 6.1.24. Example 24: Compound I-34

### (S)-22-(4-(((2-amino-4-hydroxypteridin-6-yl)methyl)amino)benzamido)-1-(1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-21-oxo-2,5,8,11,14,17-hexaoxa-20-azapentacosan-25-oic acid (Compound I-34)

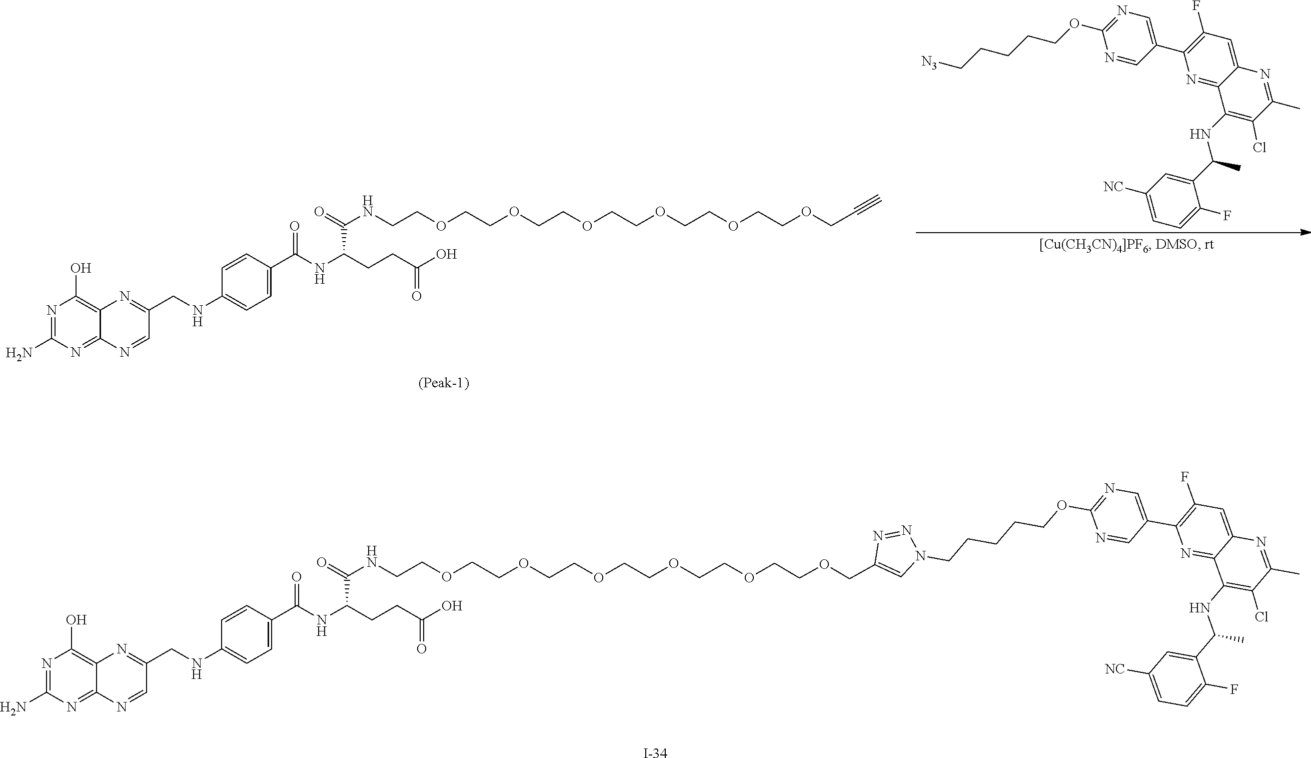

To a solution of (S)-24-(4-(((2-amino-4-hydroxypteridin-6-yl)methyl)amino)benzamido)-23-oxo-4,7,10,13,16,19-hexaoxa-22-azaheptacos-1-yn-27-oic acid (Peak-1, 4.0 eq, 0.132 g, 0.177 mmol) and (S)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.025 g, 0.0443 mmol) in dimethylsulfoxide (2 mL), tetrakis(acetonitrile)copper(I) hexafluorophosphate was added (2.8 eq, 0.0463 g, 0.124 mmol) was added and the reaction mixture was stirred at room temperature for 1 h. After completion, the reaction mixture was quenched with acetic acid (0.2 mL) and directly purified by prep HPLC (35-63% acetonitrile in water with 0.1% acetic acid). Fractions containing the desired compound were combined and lyophilized to afford (S)-22-(4-(((2-amino-4-hydroxypteridin-6-yl)methyl)amino)benzamido)-1-(1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)-21-oxo-2,5,8,11,14,17-hexaoxa-20-azapentacosan-25-oic acid (Cpd. No. I-34) as a yellow solid. Yield: 0.012 g, 20.7%; LCMS m/z 1306.74 [M+1]⁺; ¹H NMR (400 MHz, DMSO-d₆ with D₂O) δ8.85 (s, 2H), 8.58 (s, 1H), 8.01-7.90 (m, 2H), 7.79-7.78 (m, 1H), 7.65-7.58 (m, 1H), 7.55-7.53 (m, 2H), 7.21-7.10 (m, 1H), 6.57 (d, J=7.6 Hz, 1H), 6.29 (bs, 1H), 4.45-4.41 (m, 4H), 4.33 (bs, 4H), 3.44-3.36 (m, 26H), 3.16-3.14 (m, 2H), 2.24 (bs, 4H), 1.98-1.92 (m, 2H), 1.88-1.82 (m, 3H), 1.80-1.70 (m, 4H), 1.63-1.57 (m, 3H), 1.35-1.27 (m, 2H).

### 6.1.25. Example 25: Compound I-35

### (S)-4-(4-(((2-amino-4-hydroxypteridin-6-yl)methyl)amino)benzamido)-5-((2-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)amino)-5-oxopentanoic acid (Compound I-35)

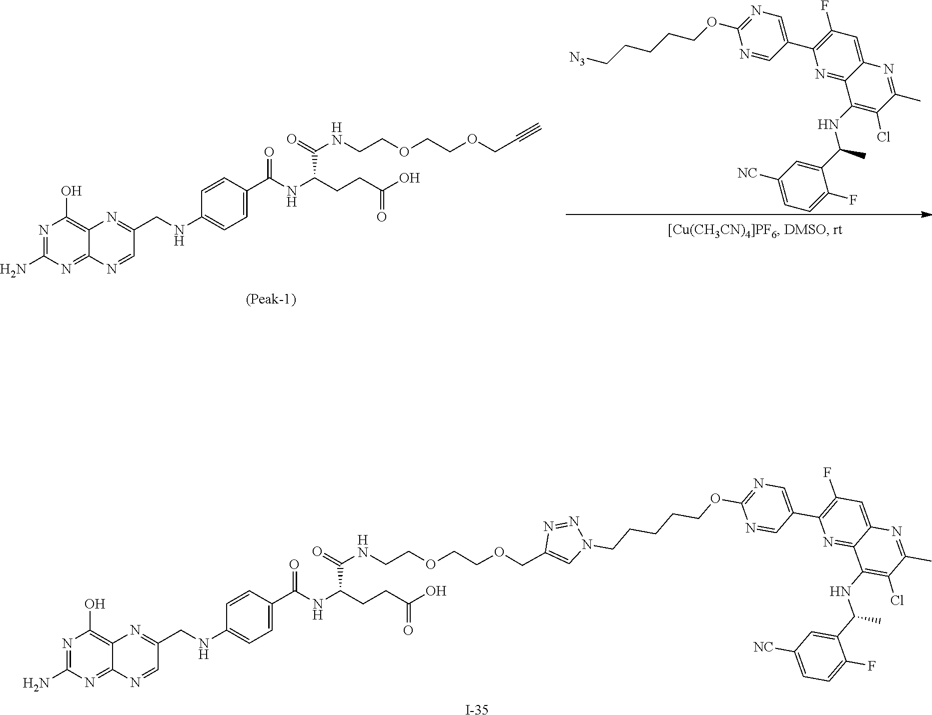

To a solution of (S)-4-(4-(((2-amino-4-hydroxypteridin-6-yl)methyl)amino)benzamido)-5-oxo-5-((2-(2-(prop-2-yn-1-yloxy)ethoxy)ethyl)amino)pentanoic acid (Peak-1, 4.0 eq, 0.092 g, 0.163 mmol) and (S)-3-(1-((6-(2-((5-azidopentyl)oxy)pyrimidin-5-yl)-3-chloro-7-fluoro-2-methyl-1,5-naphthyridin-4-yl)amino)ethyl)-4-fluorobenzonitrile (1.0 eq, 0.023 g, 0.040 mmol) in dimethylsulfoxide (2 mL), tetrakis(acetonitrile)copper(I) hexafluorophosphate was added (2.8 eq, 0.042 g, 0.114 mmol) and the reaction mixture was stirred at room temperature for 1 h. After completion, the reaction mixture was quenched with acetic acid (0.2 mL) and directly purified by prep HPLC (25-47% acetonitrile in water with 0.1% acetic acid). Fractions containing the desired compound were combined and lyophilized to afford (S)-4-(4-(((2-amino-4-hydroxypteridin-6-yl)methyl)amino)benzamido)-5-((2-(2-((1-(5-((5-(7-chloro-8-(((S)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)ethyl)amino)-5-oxopentanoic acid (Cpd. No. I-35) as a yellow solid. Yield: 0.003 g, 6.5%; LCMS m/z 566.27 [M+2]⁺⁺; ¹H NMR (400 MHz, DMSO-d₆ with D₂O) δ8.99 (s, 2H), 8.62 (s, 1H), 8.11 (d, J=11.2 Hz, 1H), 8.07 (s, 1H), 7.96-7.94 (m, 1H), 7.80-7.70 (m, 1H), 7.62 (d, J=8.4 Hz, 1H), 7.28 (t, J=8.4 Hz, 1H), 6.61 (d, J=8.4 Hz, 2H), 6.35-6.32 (m, 1H), 4.47 (s, 5H), 4.38-4.30 (m, 6H), 3.50-3.48 (m, 4H), 3.37-3.35 (m, 2H), 3.25-3.15 (m, 2H), 2.63 (bs, 4H), 2.25-2.23 (m, 4H), 1.95-1.88 (m, 5H), 1.80-1.75 (m, 4H), 1.64 (d, J=6.8 Hz, 3H), 1.40-1.30 (m, 2H).

### 6.1.26. Example 26: Compound I-36

### (S)-2-(4-(((2-amino-4-hydroxypteridin-6-yl)methyl)amino)benzamido)-3-(4-(2-((1-(5-((5-(7-chloro-8-(((R)-1-(5-cyano-2-fluorophenyl)ethyl)amino)-3-fluoro-6-methyl-1,5-naphthyridin-2-yl)pyrimidin-2-yl)oxy)pentyl)-1H-1,2,3-triazol-4-yl)methoxy)ethoxy)phenyl)propanoic acid (Compound I-36)

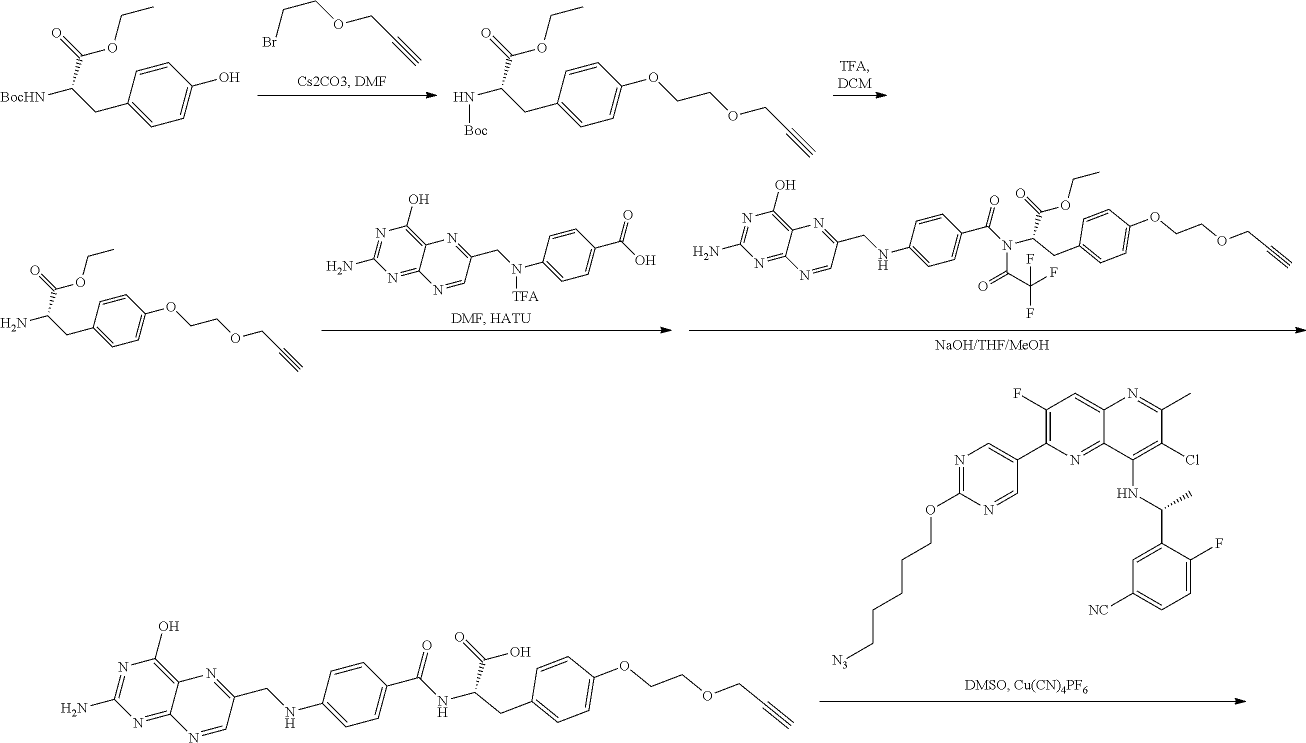

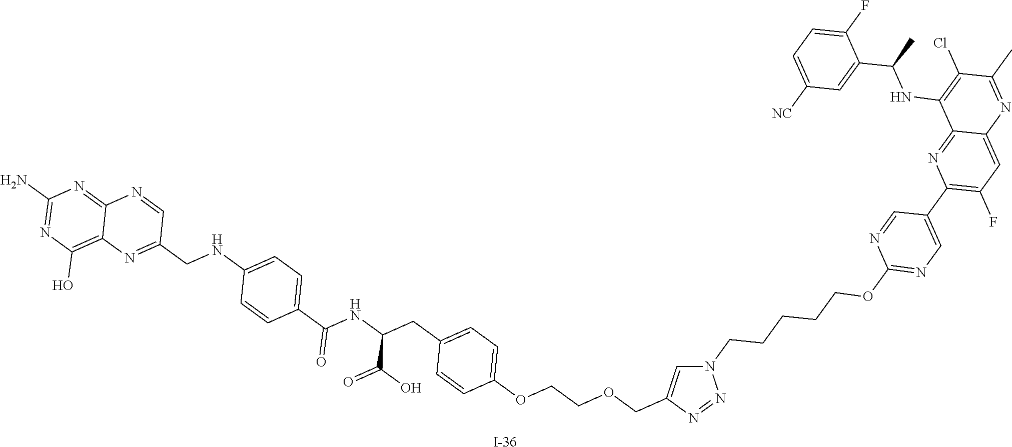

To a room temperature solution of (s)-ethyl 2-((tert-butoxycarbonyl)amino)-3-(4-hydroxyphenyl)propanoate (1.00 eq, 1000 mg, 3.23 mmol) in 8 mL of anhydrous DMF was added Cs₂CO₃ (1.50 eq, 1580 mg, 4.85 mmol) followed by 3-(2-bromoethoxy)prop-1-yne (1.50 eq, 790 mg, 4.85 mmol) and the reaction mixture was stirred at 45° C. for 20 hours. The mixture was diluted with EtOAc (150 mL) washed twice with water, once with brine solution, and the organic fraction dried over sodium sulfate and concentrated to 2 g yellow oil. The crude product was purified by column chromatography, eluting from 80 g silica gel with a gradient of 0-70% EtOAc/hexanes to give 1.2 g of a white solid. 95% yield; LCMS (m/z): 414.2 [M+Na]

Ethyl (2S)-2-(tert-butoxycarbonylamino)-3-[4-(2-prop-2-ynoxyethoxy)phenyl]propanoate (1.00 eq, 1200 mg, 3.07 mmol) in DCM (24 mL) was cooled to 0C and TFA (12 mL) as added. The resulting solution was stirred for 1 hr at 0° C. and 1 hr at RT. The solution was concentrated to a residue, which was diluted and concentrated from DCE twice. The crude oil was placed under high vacuum overnight to give 1.5 g of semi-solid. Quantitative yield; LCMS (m/z): 292.1 [M+1]

To a solution of 4-[(2-amino-4-hydroxy-pteridin-6-yl)methyl-(2,2,2-trifluoroacetyl)amino]benzoic acid (1.20 eq, 60.4 mg, 0.148 mmol) in anhydrous DMF (200 L) was added N,N-Diisopropylethylamine (3.00 eq, 0.064 mL, 0.370 mmol) and HATU (1.30 eq, 61.0 mg, 0.160 mmol) followed by a solution of ethyl (2S)-2-amino-3-[4-(2-prop-2-ynoxyethoxy)phenyl]propanoate;2,2,2-trifluoroacetic acid (1.00 eq, 50.0 mg, 0.123 mmol) and 25 μL N,N-Diisopropylethylamine in 123 μL anhydrous DMF. The mixture was stirred at room temperature for 4 hours. The crude reaction mixture was purified by HPLC eluting from a 20 mm×150 C18 column with 15-100% CH3CN/water to give 29 mg of a white solid. 34% yield; LCMS m/z=682.2 [M+1].

To a slurry of ethyl (2S)-2-[[4-[(2-amino-4-hydroxy-pteridin-6-yl)methylamino]benzoyl]-(2,2,2-trifluoroacetyl)amino]-3-[4-(2-prop-2-ynoxyethoxy)phenyl]propanoate (1.00 eq, 15.0 mg, 0.0220 mmol) in 100 μL MeOH and 100 μL THF was added 55 μL of 1N NaOH and the mixture was stirred at room temperature for 18 hours. And additional 22 μL of 1N NaOH was added, and the mixture was stirred for 20 hours. 75 μL of 1 N HCl was added to precipitate an orange solid. The solid was washed with water and dried under high-vacuum to give 10 mg of desired product. 81.5% yield; LCMS m/z: 558 [M+1]

To a solution of (2S)-2-[[4-[(2-amino-4-hydroxy-pteridin-6-yl)methylamino]benzoyl]amino]-3-[4-(2-prop-2-ynoxyethoxy)phenyl]propanoic acid (1.00 eq, 10.0 mg, 0.0179 mmol) and 3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (1.00 eq, 10.1 mg, 0.0179 mmol) in dimethylsulfoxide 200 uL, tetrakis(acetonitrile)copper(I) hexafluorophosphate (0.500 eq, 3.4 mg, 0.00894 mmol) was added and reaction mixture was stirred at room temperature for 1 h. After completion, reaction mixture was directly purified by prep HPLC (eluting from a C18 column with 35-100% acetonitrile in water with 0.1% FA). Fractions containing the desired product were combined and lyophilized to dryness to afford the desired product (Compound I-36). Yield: 5.8 mg, 27%; LCMS m/z 1221.3 [M+1].

### 6.1.27. Example 27: Compound I-37

### (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoic acid (Compound I-37)

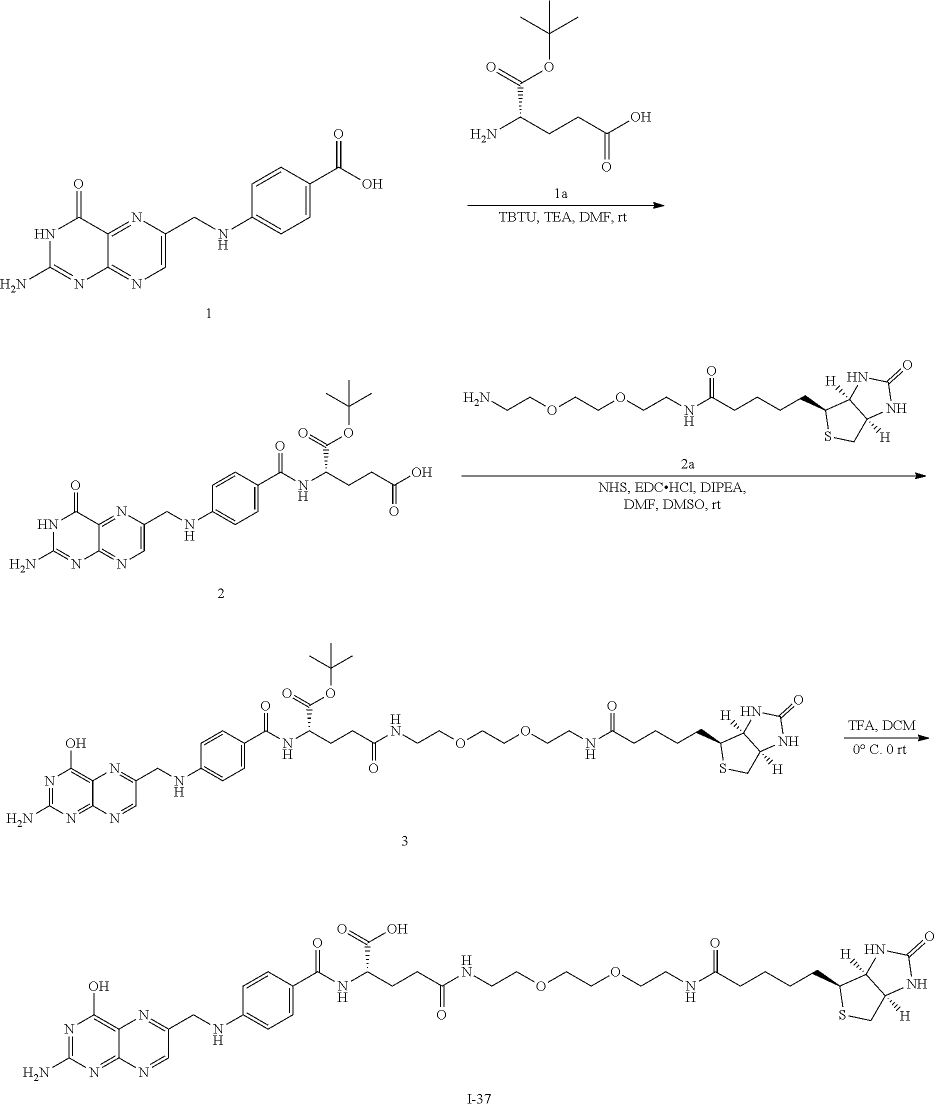

To a suspension of 4-(((2-amino-4-oxo-3,4-dihydropteridin-6-yl)methyl)amino)benzoic acid (pteroic acid) (1, 1.0 eq, 4.0 g, 12.8 mmol) in N,N-dimethylformamide (80 mL), triethylamine (2.0 eq, 3.57 mL, 25.6 mmol) and 2-(1H-Benzotriazole-1-yl)-1,1,3,3-tetramethylaminium tetrafluoroborate (TBTU) (1.0 eq, 4.11 g, 12.8 mmol) were added and reaction mixture was stirred at room temperature for 2.5 h. In another round bottom flask, a suspension of (S)-4-amino-5-(tert-butoxy)-5-oxopentanoic acid (1a, 1.15 eq, 2.99 g, 14.7 mmol) and triethylamine (1.2 eq, 2.13 mL, 15.4 mmol) in N,N-dimethylformamide (60 mL) was prepared. The active ester was added to this suspension and reaction mixture was stirred at room temperature for 16 h. After completion (monitored by LCMS), reaction mixture was concentrated, ethyl acetate was added and stirred for 30 minutes. The resulting brown solid was collected by filtration, washed with chloroform and dried to get crude (4.0 g). This crude was dissolved in aqueous 1N sodium hydroxide solution (16 mL) and water (24 mL) which was purified by prep. HPLC (22-55% acetonitrile in water with 5 mM ammonium bicarbonate). Fractions containing the desired product were combined and lyophilized to dryness to afford (S)-4-(4-(((2-amino-4-oxo-3,4-dihydropteridin-6-yl)methyl)amino)benzamido)-5-(tert-butoxy)-5-oxopentanoic acid (2) as a yellow solid. Yield: 1.3 g, 20.4%; LCMS m/z 498.13 [M+1]⁺.

To a solution of (S)-4-(4-(((2-amino-4-oxo-3,4-dihydropteridin-6-yl)methyl)amino)benzamido)-5-(tert-butoxy)-5-oxopentanoic acid (2, 1.0 eq, 0.100 g, 0.201 mmol) in N,N-dimethylformamide (2 mL) and dimethyl sulfoxide (2 mL), N-hydroxysuccinimide (1.5 eq, 0.034 g, 0.302 mmol), N-(3-Dimethylaminopropyl)-N′-ethylcarbodiimide hydrochloride (EDC·HCl) (1.5 eq, 0.057 g, 0.302 mmol) and N,N-diisopropylethylamine (3.0 eq, 0.1 mL, 0.603 mmol) were added and reaction mixture was stirred at room temperature for 30 minutes. Then, N-(2-(2-(2-aminoethoxy)ethoxy)ethyl)-5-((3aS,4S,6aR)-2-oxohexahydro-1H-thieno[3,4-d]imidazol-4-yl)pentanamide (2a, 1.2 eq, 0.090 g, 0.241 mmol) was added and reaction mixture was stirred at room temperature for 16 h. After that, reaction mixture was directly purified by prep HPLC (20-32% acetonitrile in water with 0.1% acetic acid). Fractions containing the desired product were combined and lyophilized to dryness to afford tert-butyl (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoate (3) as a yellow solid. Yield: 0.007 g, 3.68%; LCMS m/z 854.51 [M+1]⁺.

A solution of tert-butyl (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoate (3, 1.0 eq, 0.006 g, 0.007 mmol) in dichloromethane (0.5 mL) was cooled at 0° C., trifluoroacetic acid (0.5 ml) was added and reaction mixture was stirred at room temperature for 3 h. After completion, reaction mixture was concentrated to get crude which was purified by prep HPLC (10-30% acetonitrile in water with 0.1% TFA). Fractions containing the desired product were combined and lyophilized to dryness to afford (S)-2-(4-(((2-amino-4-hydroxypteridin-6-yl)methyl)amino)benzamido)-5,16-dioxo-20-((3aS,4S,6aR)-2-oxohexahydro-1H-thieno[3,4-d]imidazol-4-yl)-9,12-dioxa-6,15-diazaicosanoic acid (Cpd. No. I-37) as a yellow solid. Yield: 0.0015 g, 27.27%; LCMS m/z 798.56 [M+1]⁺; ¹H NMR (400 MHz, DMSO-d6 with D₂O) δ8.62 (s, 1H), 7.59 (d, J=8.8 Hz, 2H), 6.62 (d, J=8.8 Hz, 2H), 4.47 (s, 2H), 4.33-4.29 (m, 1H), 4.19-4.17 (m, 1H), 4.14-4.11 (m, 1H), 3.42 (s, 4H), 3.36-3.32 (m, 4H), 3.16-3.13 (m, 4H), 3.07-3.05 (m, 1H), 2.80-2.75 (m, 1H), 2.57-2.54 (m, 1H), 2.16-2.12 (m, 2H), 2.05-2.02 (m, 3H), 1.92-1.81 (m, 1H), 1.65-1.50 (m, 1H), 1.46-1.43 (m, 3H), 1.25-1.23 (m, 2H).

### 6.1.28. Example 28: Compound I-38

### N2-(4-(((2-amino-4-hydroxypteridin-6-yl)methyl)amino)benzoyl)-N5-(2-(2-((1-(2-(2-(2-(5-((3aS,4S,6aR)-2-oxohexahydro-1H-thieno[3,4-d]imidazol-4-yl)pentanamido)ethoxy)ethoxy)ethyl)-1H-1,2,3-triazol-4-yl)methoxy)ethoxy)ethyl)-L-glutamine (Compound I-38)

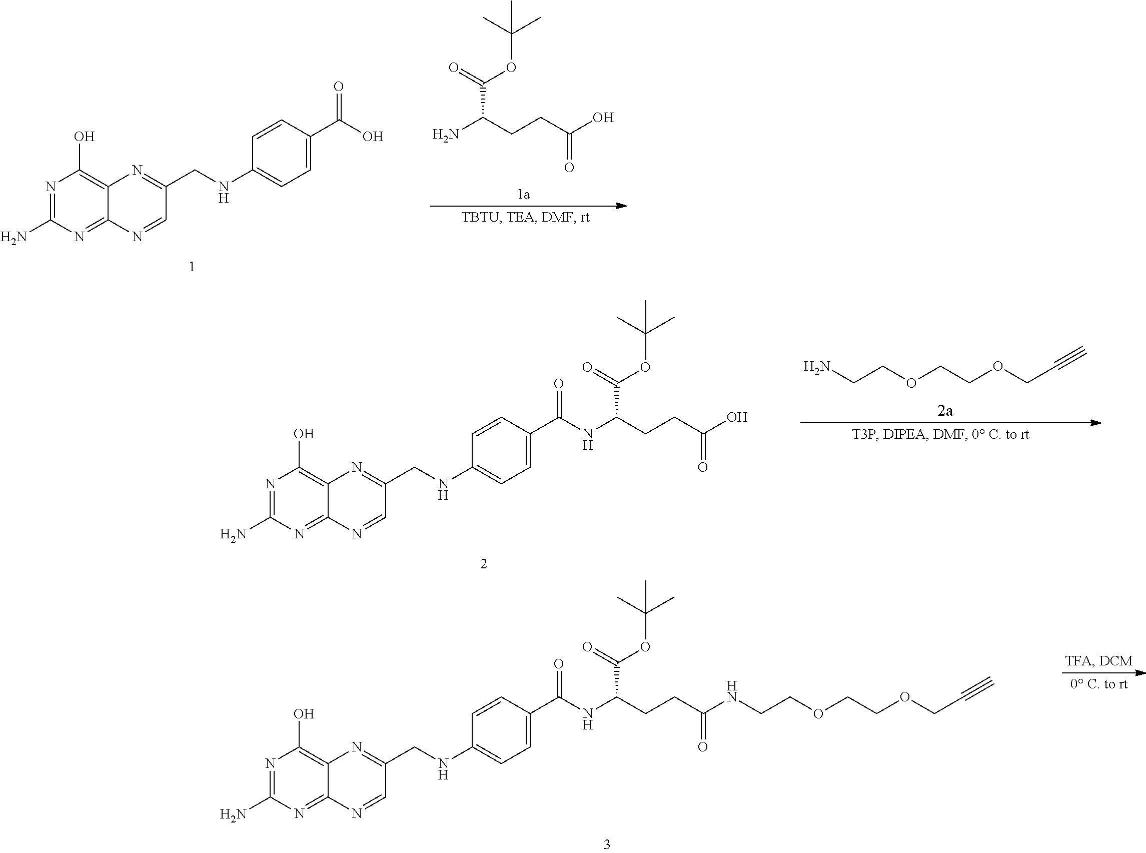

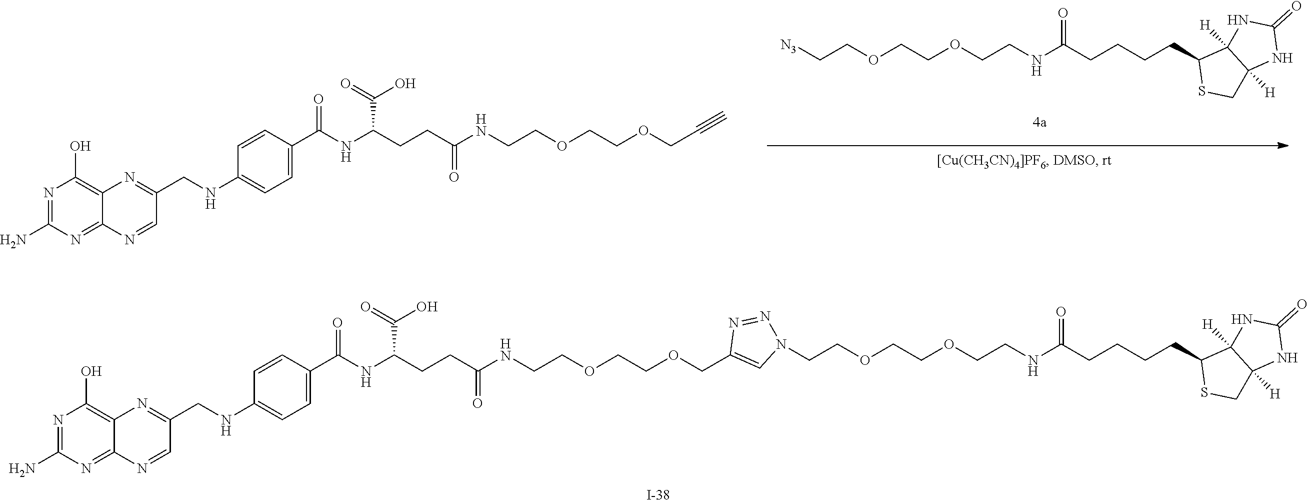

To a suspension of 4-(((2-amino-4-hydroxypteridin-6-yl)methyl)amino)benzoic acid (pteroic acid) (1, 1.0 eq, 4.0 g, 12.8 mmol) in N,N-dimethylformamide (80.0 mL), triethylamine (2.0 eq, 3.57 mL, 25.6 mmol) and 2-(1H-Benzotriazole-1-yl)-1,1,3,3-tetramethylaminium tetrafluoroborate (TBTU) (1.0 eq, 4.11 g, 12.8 mmol) were added and reaction mixture was stirred at room temperature for 2.5 h. In another round bottom flask, a suspension of (S)-4-amino-5-(tert-butoxy)-5-oxopentanoic acid (1a, 1.15 eq, 2.99 g, 14.7 mmol) and triethylamine (1.2 eq, 2.13 mL, 15.4 mmol) in N,N-dimethylformamide (60 mL) was prepared. The active ester was added to this suspension and reaction mixture was stirred at room temperature for 16 h. After completion, N,N-dimethylformamide was concentrated, ethyl acetate was added and stirred for 30 minutes. The solid was collected by filtration, washed with chloroform to get crude (4.0 g). This crude was dissolved in aqueous 1N sodium hydroxide solution (16 mL) and water (24 mL) which was purified by prep. HPLC (22-55% acetonitrile in water with 5 mM ammonium bicarbonate). Fractions containing the desired product were combined and lyophilized to dryness to afford (S)-4-(4-(((2-amino-4-hydroxypteridin-6-yl)methyl)amino)benzamido)-5-(tert-butoxy)-5-oxopentanoic acid (2) as a yellow solid. Yield: 1.3 g, 20.4%; LCMS m/z 498.13 [M+1]⁺.

A solution of (S)-4-(4-(((2-amino-4-hydroxypteridin-6-yl)methyl)amino)benzamido)-5-(tert-butoxy)-5-oxopentanoic acid (2, 1.0 eq, 0.100 g, 0.201 mmol) in N,N-dimethylformamide (2.0 mL) was cooled at 0° C., 2-(2-(prop-2-yn-1-yloxy)ethoxy)ethan-1-amine (2a, 1.5 eq, 0.043 g, 0.302 mmol), N,N-diisopropylethylamine (3.0 eq, 0.1 mL, 0.603 mmol) and 1-propanephosphonic anhydride (T3P) (1.5 eq, 0.18 mL, 0.302 mmol) were added and reaction mixture was stirred at room temperature for 72 h. After that, reaction mixture was directly purified by prep HPLC (20-32% acetonitrile in water with 0.1% acetic acid). Fractions containing the desired product were combined and lyophilized to dryness to afford tert-butyl N2-(4-(((2-amino-4-hydroxypteridin-6-yl)methyl)amino)benzoyl)-N5-(2-(2-(prop-2-yn-1-yloxy)ethoxy)ethyl)-L-glutaminate (3) as a yellow solid. Yield: 0.014 g, 11.19%; LCMS m/z 623.19 [M+1]⁺.

A solution of tert-butyl N2-(4-(((2-amino-4-hydroxypteridin-6-yl)methyl)amino)benzoyl)-N5-(2-(2-(prop-2-yn-1-yloxy)ethoxy)ethyl)-L-glutaminate (1.0 eq, 0.014 g, 0.022 mmol) in dichloromethane (0.5 mL) was cooled at 0° C., trifluoroacetic acid (0.5 mL) was added and reaction mixture was stirred at room temperature for 2 h. After completion, reaction mixture was concentrated, azeotroped with dichloromethane (2-3 times) and dried to afford N2-(4-(((2-amino-4-hydroxypteridin-6-yl)methyl)amino)benzoyl)-N5-(2-(2-(prop-2-yn-1-yloxy)ethoxy)ethyl)-L-glutamine as a yellow solid. Yield: 0.012 g (Crude); LCMS m/z 567.14 [M+1]⁺.

To a solution of N2-(4-(((2-amino-4-hydroxypteridin-6-yl)methyl)amino)benzoyl)-N5-(2-(2-(prop-2-yn-1-yloxy)ethoxy)ethyl)-L-glutamine (1.0 eq, 0.012 g, 0.021 mmol) and N-(2-(2-(2-azidoethoxy)ethoxy)ethyl)-5-((3aS,4S,6aR)-2-oxohexahydro-1H-thieno[3,4-d]imidazol-4-yl)pentanamide (4a, 1.0 eq, 0.008 g, 0.021 mmol) in dimethylsulfoxide (0.5 mL), tetrakis(acetonitrile)copper(I) hexafluorophosphate (2.8 eq., 0.022 g, 0.059 mmol) was added and reaction mixture was stirred at room temperature for 1 h. After completion, reaction mixture was directly purified by prep HPLC (13-25% acetonitrile in water with 0.1% TFA). Fractions containing the desired product were combined and lyophilized to dryness to afford N2-(4-(((2-amino-4-hydroxypteridin-6-yl)methyl)amino)benzoyl)-N5-(2-(2-((1-(2-(2-(2-(5-((3aS,4S,6aR)-2-oxohexahydro-1H-thieno[3,4-d]imidazol-4-yl)pentanamido)ethoxy)ethoxy)ethyl)-1H-1,2,3-triazol-4-yl)methoxy)ethoxy)ethyl)-L-glutamine (Cpd. No. I-38) as a yellow solid. Yield: 0.003 g, 14.15%; LCMS m/z 967.67 [M+1]+; ¹H NMR (400 MHz, DMSO-d6) δ12.46 (s, 1H), 11.40 (s, 1H), 8.64 (s, 1H), 8.19 (d, J=7.6 Hz, 1H), 8.04 (s, 1H), 7.91 (t, J=6.0 Hz, 1H), 7.80 (t, J=4.8 Hz, 1H), 7.64 (d, J=8.8 Hz, 2H), 6.93 (t, J=7.6 Hz, 1H), 6.63 (d, J=6.0 Hz, 1H), 6.42 (s, 1H), 6.36 (s, 1H), 4.50-4.47 (m, 6H), 4.32-4.25 (m, 2H), 4.13-4.10 (m, 1H), 3.79 (t, J=5.2 Hz, 2H), 3.59-3.45 (m, 10H), 3.39-3.35 (m, 4H), 3.20-3.3.15 (m, 5H), 3.10-3.07 (m, 1H), 2.82-2.78 (m, 1H), 2.24-2.18 (m, 3H), 2.05 (t, J=7.2 Hz, 2H), 1.95-1.85 (m, 1H), 1.61-1.57 (m, 1H), 1.52-1.42 (m, 3H), 1.28-1.23 (m, 2H).

### 6.1.29. Example 29: Compound I-39

### Synthesis of (S)-30-(4-(((2-amino-4-hydroxypteridin-6-yl)methyl)amino)benzamido)-27-oxo-1-(1-(2-(2-(2-(5-((3aS,4S,6aR)-2-oxohexahydro-1H-thieno[3,4-d]imidazol-4-yl)pentanamido)ethoxy)ethoxy)ethyl)-1H-1,2,3-triazol-4-yl)-2,5,8,11,14,17,20,23-octaoxa-26-azahentriacontan-31-oic acid (Compound I-39)

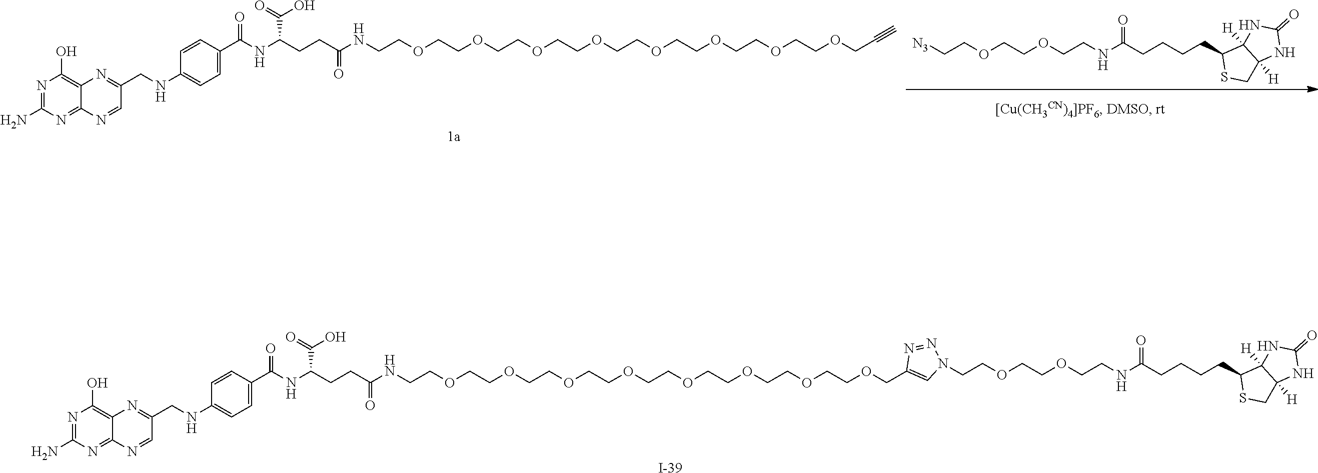

To a solution of (S)-32-(4-(((2-amino-4-hydroxypteridin-6-yl)methyl)amino)benzamido)-29-oxo-4,7,10,13,16,19,22,25-octaoxa-28-azatritriacont-1-yn-33-oic acid (1.0 eq, 0.040 g, 0.081 mmol) and N-(2-(2-(2-azidoethoxy)ethoxy)ethyl)-5-((3aS,4S,6aR)-2-oxohexahydro-1H-thieno[3,4-d]imidazol-4-yl)pentanamide (1a, 1.05 eq, 0.0202 g, 0.0505 mmol) in anhydrous dimethylsulfoxide (1.5 mL), tetrakis(acetonitrile)copper(I) hexafluorophosphate was added (2.8 eq, 0.0405 g, 0.226 mmol) and reaction mixture was stirred at room temperature for 0.5 h. After completion, the reaction mixture was quenched with acetic acid (0.2 mL) and directly purified by prep HPLC (20-42% acetonitrile in water with 0.1% trifluoroacetic acid). All the fractions containing desired compound were combined and lyophilized to afford (S)-30-(4-(((2-amino-4-hydroxypteridin-6-yl)methyl)amino)benzamido)-27-oxo-1-(1-(2-(2-(2-(5-((3aS,4S,6aR)-2-oxohexahydro-H-thieno[3,4-d]imidazol-4-yl)pentanamido)ethoxy)ethoxy)ethyl)-1H-1,2,3-triazol-4-yl)-2,5,8,11,14,17,20,23-octaoxa-26-azahentriacontan-31-oic acid (Cpd. No. I-39) as a yellow solid. Yield: 0.020 g, 33.73%; LCMS m/z 616.73 [M+2]⁺⁺; ¹H NMR (400 MHz, DMSO-d6 with D₂O) δ 8.66 (s, 1H), 8.01 (s, 1H), 7.62 (d, J=8.4 Hz, 2H), 6.63 (d, J=8.8 Hz, 2H), 4.48-4.46 (m, 6H), 4.31-4.28 (m, 1H), 4.25-4.23 (m, 1H), 4.13-4.10 (m, 1H), 3.78 (t, J=4.8 Hz, 2H), 3.53-3.49 (m, 6H), 3.47-3.45 (m, 26H), 3.37-3.32 (m, 4H), 3.17-3.13 (m, 4H), 3.08-3.06 (m, 1H), 2.80 (dd, J=12.6 and 4.8 Hz, 1H), 2.58 (s, 1H), 2.24-2.16 (m, 2H), 2.06-2.00 (m, 3H), 1.92-1.86 (m, 1H), 1.59-1.56 (m, 1H), 1.47-1.39 (m, 3H), 1.31-1.24 (m, 2H).

### 6.1.30. Example 30: Compound I-40

### N2-(4-(((2-amino-4-hydroxypteridin-6-yl)methyl)amino)benzoyl)-N5-(2-(2-((1-(2-((2-(5-((3aS,4S,6aR)-2-oxohexahydro-1H-thieno[3,4-d]imidazol-4-yl)pentanamido)ethyl)disulfaneyl)ethyl)-1H-1,2,3-triazol-4-yl)methoxy)ethoxy)ethyl)-L-glutamine (Compound I-40)

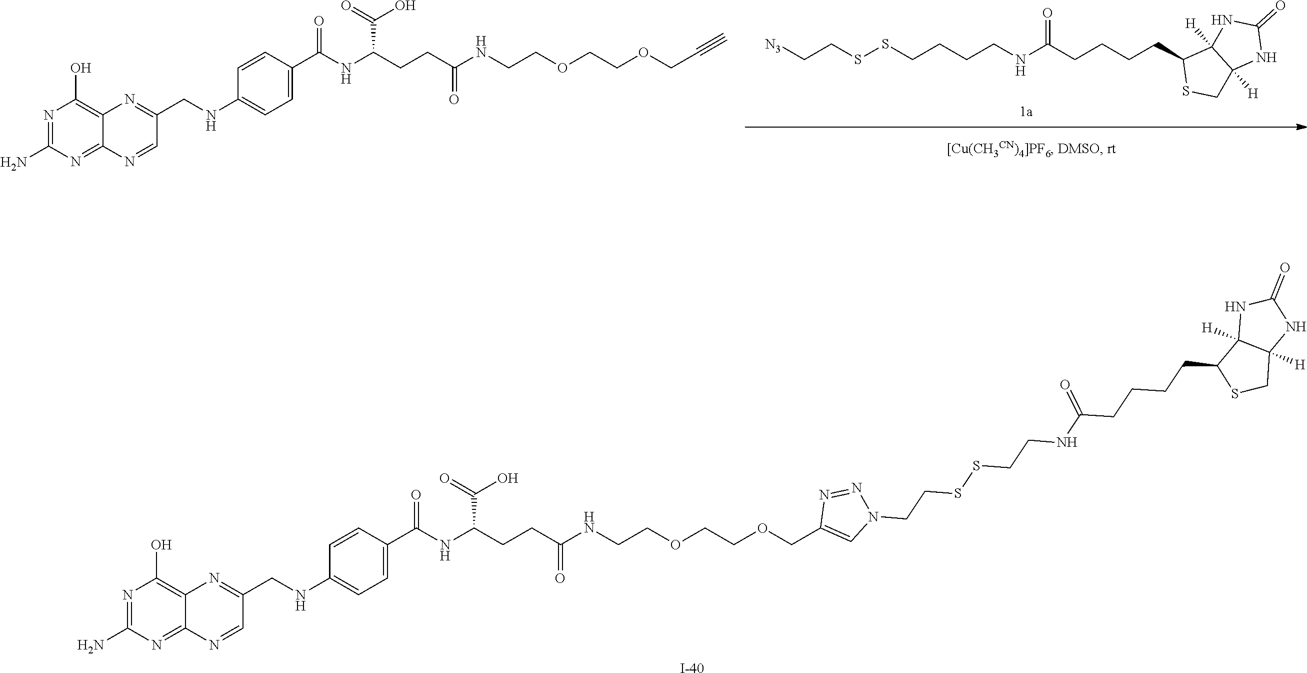

To a solution of N2-(4-(((2-amino-4-hydroxypteridin-6-yl)methyl)amino)benzoyl)-N5-(2-(2-(prop-2-yn-1-yloxy)ethoxy)ethyl)-L-glutamine (1.0 eq, 0.040 g, 0.007 mmol) and N-(2-((2-azidoethyl)disulfaneyl)ethyl)-5-((3aS,4S,6aR)-2-oxohexahydro-1H-thieno[3,4-d]imidazol-4-yl)pentanamide (1a, 1.0 eq, 0.028 g, 0.007 mmol) in anhydrous dimethylsulfoxide (1.5 mL), tetrakis(acetonitrile)copper(I) hexafluorophosphate (2.8 eq, 0.073 g, 0.198 mmol) was added and reaction mixture was stirred at room temperature for 30 minutes. After completion, the reaction mixture was quenched with acetic acid (0.2 mL) and directly purified by prep HPLC (14-25% acetonitrile in water with 0.1% trifluoroacetic acid). All the fractions containing desired compound were combined and lyophilized to afford N2-(4-(((2-amino-4-hydroxypteridin-6-yl)methyl)amino)benzoyl)-N5-(2-(2-((1-(2-((2-(5-((3aS,4S,6aR)-2-oxohexahydro-1H-thieno[3,4-d]imidazol-4-yl)pentanamido)ethyl)disulfaneyl)ethyl)-1H-1,2,3-triazol-4-yl)methoxy)ethoxy)ethyl)-L-glutamine as a yellow solid. Yield: 0.024 g, 35%; LCMS m/z 486.59 [M+2]**; ¹H-NMR (400 MHz, DMSO-d6 with D₂O) δ 8.66 (s, 1H), 8.06 (s, 1H), 7.62 (d, J=8.8 Hz, 2H), 6.63 (d, J=8.8 Hz, 2H), 4.61 (t, J=6.8 Hz, 2H), 4.49 (d, J=9.2 Hz, 4H), 4.30-4.28 (m, 1H), 4.29-4.26 (m, 1H), 4.13-4.12 (m, 1H), 3.55-3.45 (m, 4H), 3.34 (t, J=5.6 Hz, 2H), 3.28 (t, J=6.4 Hz, 2H), 3.19-3.13 (m, 4H), 3.10-3.05 (m, 1H), 2.79-2.74 (m, 3H), 2.57 (s, 1H), 2.18-2.16 (m, 2H), 2.07-2.03 (m, 3H), 1.95-1.85 (m, 1H), 1.65-1.55 (m, 1H), 1.53-1.37 (m, 3H), 1.32-1.22 (m, 2H).

### 6.1.31. Example 31: Compound I-78

### (S)-2-(4-(((2-amino-4-oxo-3,4-dihydropteridin-6-yl)methyl)(methyl)amino)benzamido)-5-(4-(14-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-14-oxo-3,6,9,12-tetraoxatetradecyl)piperazin-1-yl)-5-oxopentanoic acid (Compound I-78)

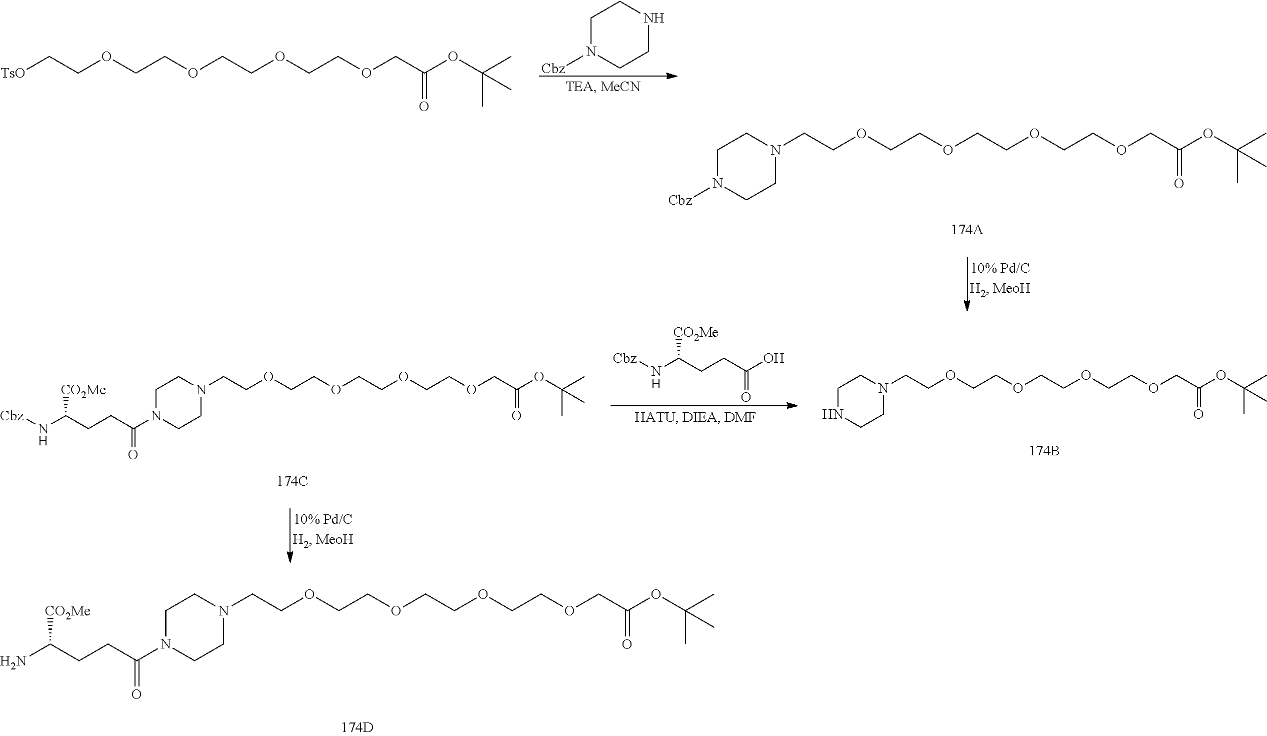

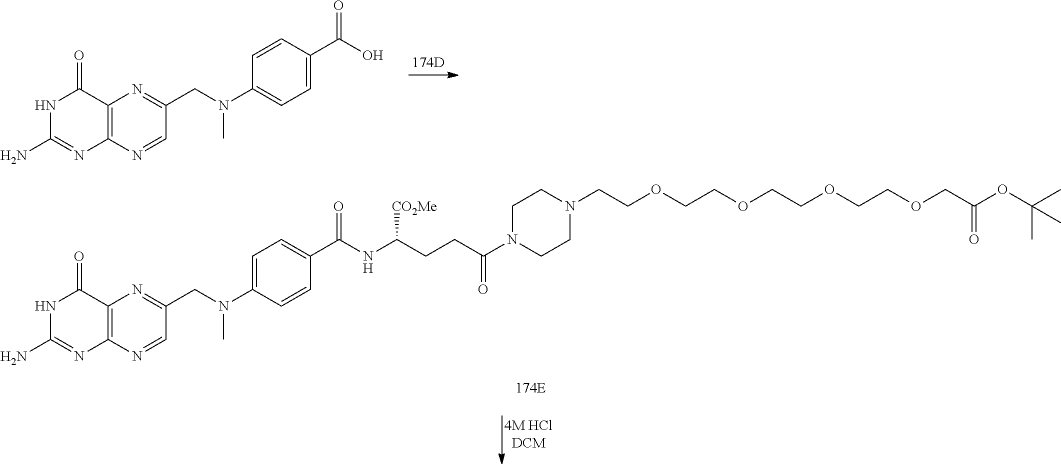 [SMILES: CN(Cc1cnc2nc(N)[nH]c(=O)c2n1)c1ccc(C(=O)O)cc1 ; COC(=O)[C@H](CCC(=O)N1CCN(CCOCCOCCOCCOCC(=O)OC(C)(C)C)CC1)NC(=O)c1ccc(N(C)Cc2cnc3nc(N)[nH]c(=O)c3n2)cc1]

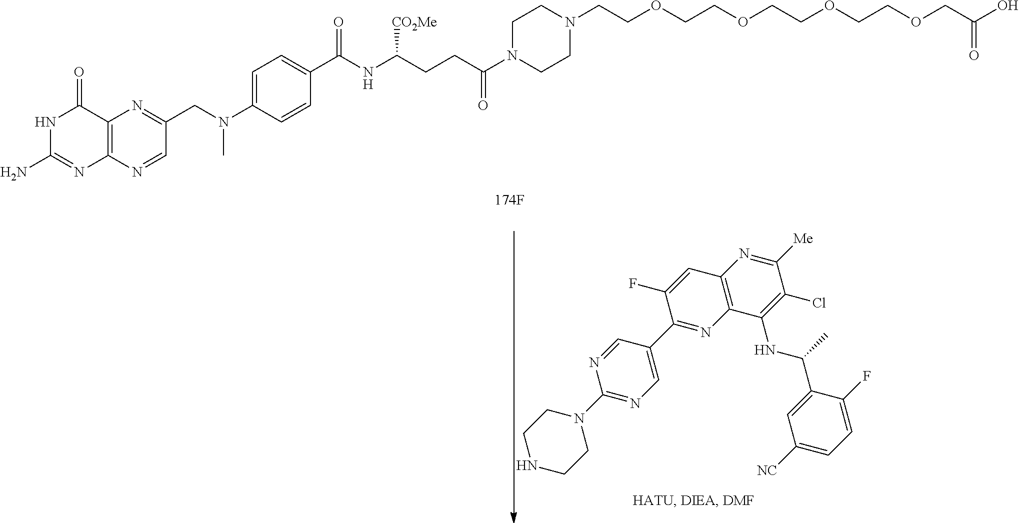 [SMILES: COC(=O)[C@H](CCC(=O)N1CCN(CCOCCOCCOCCOCC(=O)O)CC1)NC(=O)c1ccc(N(C)Cc2cnc3nc(N)[nH]c(=O)c3n2)cc1 ; Cc1nc2cc(F)c(-c3cnc(N4CCNCC4)nc3)nc2c(N[C@H](C)c2cc(C#N)ccc2F)c1Cl]

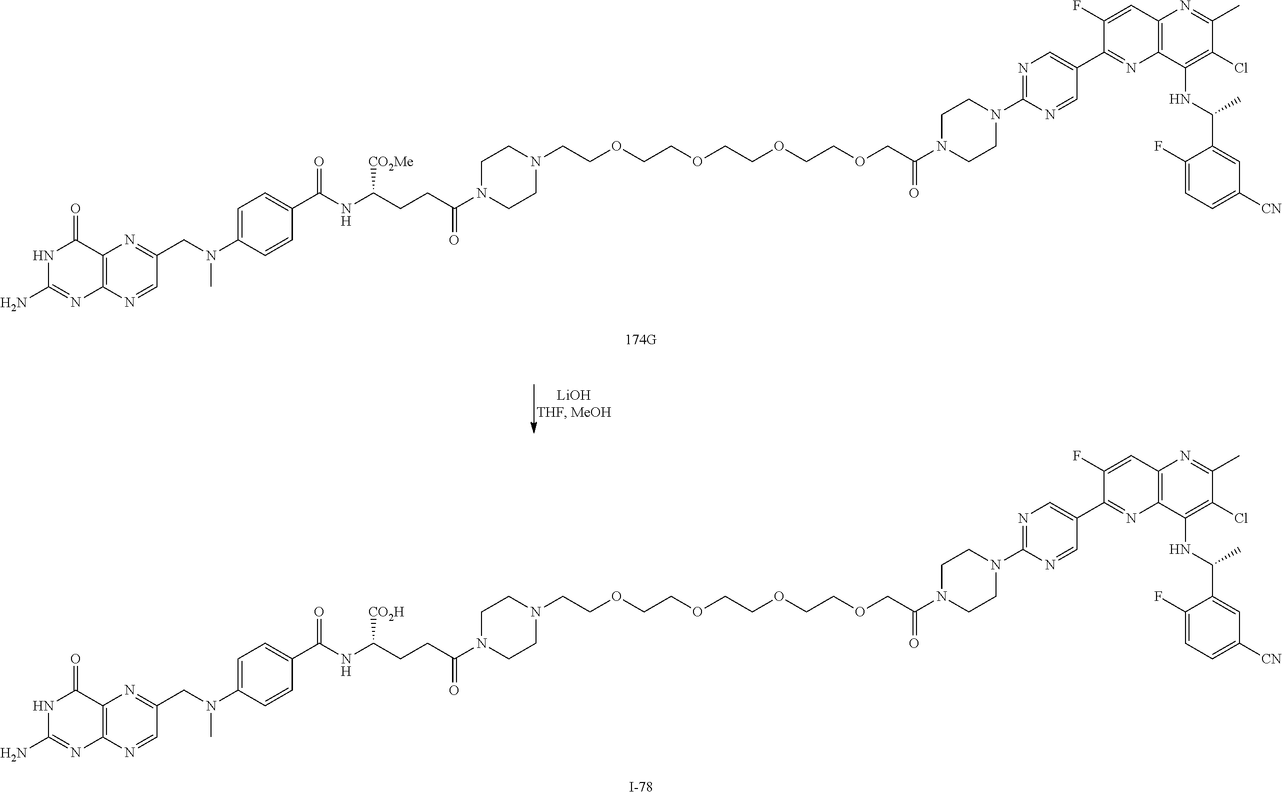

To a mixture of benzyl piperazine-1-carboxylate (1.10 eq, 53.4 mg, 0.242 mmol) and tert-butyl 2-[2-[2-[2-[2-(p-tolylsulfonyloxy)ethoxy]ethoxy]ethoxy]ethoxy]acetate (1.00 eq, 102 mg, 0.22 mmol) in MeCN (1 mL) was added N,N-diethylethanamine (2.00 eq, 0.061 mL, 0.44 mmol). The mixture was stirred at 70° C. overnight. The mixture was concentrated and purified by column (0-10% MeOH/DCM) to give 174A as clear syrup (85.7 mg, yield: 76%). LCMS m/z 511.5 [M+H]⁺.

To a mixture of benzyl 4-[2-[2-[2-[2-(2-tert-butoxy-2-oxo-ethoxy)ethoxy]ethoxy]ethoxy]ethyl]piperazine-1-carboxylate (174A, 1.00 eq, 85.7 mg, 0.17 mmol) in MeOH (4 mL) was added 10% Pd/C (20 mg). The mixture was stirred under H₂ at room temperature for 2 h, filtered, and concentrated to give 174B as clear syrup (66.5 mg, yield: 105%). LCMS m/z 377.4 [M+H]⁺.

To a mixture of (4S)-4-(benzyloxycarbonylamino)-5-methoxy-5-oxo-pentanoic acid (1.00 eq, 36.7 mg, 0.12 mmol) and tert-butyl 2-[2-[2-[2-(2-piperazin-1-ylethoxy)ethoxy]ethoxy]ethoxy]acetate (174B, 1.00 eq, 46.8 mg, 0.12 mmol) in DMF (1 mL) were added N, N-Diisopropylethylamine (3.00 eq, 0.065 mL, 0.37 mmol) and HATU (1.2 eq, 56.7 mg, 0.15 mmol). The mixture was stirred at room temperature for 1 h, concentrated, diluted with water, extracted with EtOAc (2×). The combined organic layers were washed with brine (1×), dried, concentrated, and purified by column (0-40% EtOAc/hexane, 0-10% MeOH/DCM) to give 174C as clear syrup (62.2 mg, yield: 77%). LCMS m/z 654.3 [M+H]f.

To a mixture of methyl (2S)-2-(benzyloxycarbonylamino)-5-[4-[2-[2-[2-[2-(2-tert-butoxy-2-oxo-ethoxy)ethoxy]ethoxy]ethoxy]ethyl]piperazin-1-yl]-5-oxo-pentanoate (174C, 1.00 eq, 62.2 mg, 0.095 mmol) in MeOH (4 mL) was added 10% Pd/C (20 mg). The mixture was stirred at room temperature under H₂ for 2 h, filtered, and concentrated to give 174D as clear syrup (48.8 mg, yield: 92%). LCMS m/z 520.3 [M+H]⁺.

To a mixture of 4-[(2-amino-4-oxo-3H-pteridin-6-yl)methyl-methyl-amino]benzoic acid; hydrobromide (1.00 eq, 19.1 mg, 0.047 mmol) and methyl (2S)-2-amino-5-[4-[2-[2-[2-[2-(2-tert-butoxy-2-oxo-ethoxy)ethoxy]ethoxy]ethoxy]ethyl]piperazin-1-yl]-5-oxo-pentanoate (174D, 1.00 eq, 24.4 mg, 0.047 mmol) in DMF (0.6 mL) were added N, N-Diisopropylethylamine (6.00 eq, 0.049 mL, 0.28 mmol) and HATU (1.2 eq, 21.4 mg. 0.056 mmol). The mixture was stirred at room temperature for 1 h and at 40° C. for 1 h. The mixture was purified by prep. HPLC (20-55% MeCN/water with 0.1% formic acid) to give 174E as a yellow solid (13.3 mg, yield: 34%). LCMS m/z 828.6 [M+H]f.

To a mixture of methyl (2S)-2-[[4-[(2-amino-4-oxo-3H-pteridin-6-yl)methyl-methyl-amino]benzoyl]amino]-5-[4-[2-[2-[2-[2-(2-tert-butoxy-2-oxo-ethoxy)ethoxy]ethoxy]ethoxy]ethyl]piperazin-1-yl]-5-oxo-pentanoate (174E, 1.00 eq, 13.3 mg, 0.016 mmol) in DCM (0.1 mL) was added 4N HCl in dioxane (0.5 mL). The mixture was stirred at room temperature for 2 h, concentrated, and lyophilized to give 174F dihydrochloride as a yellow solid (13.3 mg, yield: 98%). LCMS m/z 772.3 [M+H]⁺.

To a mixture of 2-[2-[2-[2-[2-[4-[(4S)-4-[[4-[(2-amino-4-oxo-3H-pteridin-6-yl)methyl-methyl-amino]benzoyl]amino]-5-methoxy-5-oxo-pentanoyl]piperazin-1-yl]ethoxy]ethoxy]ethoxy]ethoxy]acetic acid; dihydrochloride (174F, 1.00 eq, 13.3 mg, 0.016 mmol) and 3-[(1R)-1-[[3-chloro-7-fluoro-2-methyl-6-(2-piperazin-1-ylpyrimidin-5-yl)-1,5-naphthyridin-4-yl]amino]ethyl]-4-fluoro-benzonitrile;ihydrochloride (1.10 eq, 10.3 mg, 0.017 mmol) in DMF (0.4 mL) were added DIEA (10 eq, 27.4 uL, 0.16 mmol) and HATU (1.2 eq, 7.2 mg, 0.019 mmol). The mixture was purified by prep. HPLC (20-55% MeCN/H₂O with 0.1% formic acid) to give 174G as a yellow solid (11.1 mg, yield: 55%). LCMS m/z 1274.3 [M+H]f.

To a mixture of methyl (2S)-2-[[4-[(2-amino-4-oxo-3H-pteridin-6-yl)methyl-methyl-amino]benzoyl]amino]-5-[4-[2-[2-[2-[2-[2-[4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]piperazin-1-yl]-2-oxo-ethoxy]ethoxy]ethoxy]ethoxy]ethyl]piperazin-1-yl]-5-oxo-pentanoate (174G, 1.00 eq, 11.1 mg, 0.0087 mmol) in THF (0.3 mL) and MeOH (0.2 mL) was added 1N LiOH (17.4 mL, 0.017 mmol). The mixture was stirred at room temperature for 40 minutes and more 1N LiOH (8.7 mL, 0.0087 mmol) was added. The mixture was stirred at room temperature for 50 minutes and more 1N LiOH (8.7 mL, 0.0097 mmol) was added. The mixture was stirred at room temperature for 30 minutes and more 1N LiOH (2 mL, 0.002 mmol) was added. The mixture was stirred at room temperature for 30 minutes, acidified with formic acid (3 mL) and concentrated to remove organic solvent, and purified by prep. HPLC (20-40% MeCN/water with 0.1% formic acid) to give I-78 as a yellow solid (7 mg, yield: 64%). LCMS 631.0 [M/2+H]⁺.

### 6.1.32. Example 32: Compound I-79

### (S)-2-(4-(((2-amino-4-hydroxyquinazolin-6-yl)methyl)thio)benzamido)-4-(1-(20-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-20-oxo-3,6,9,12,15,18-hexaoxaicosyl-1H-1,2-triazol-4-yl)butanoic acid (Compound I-79)

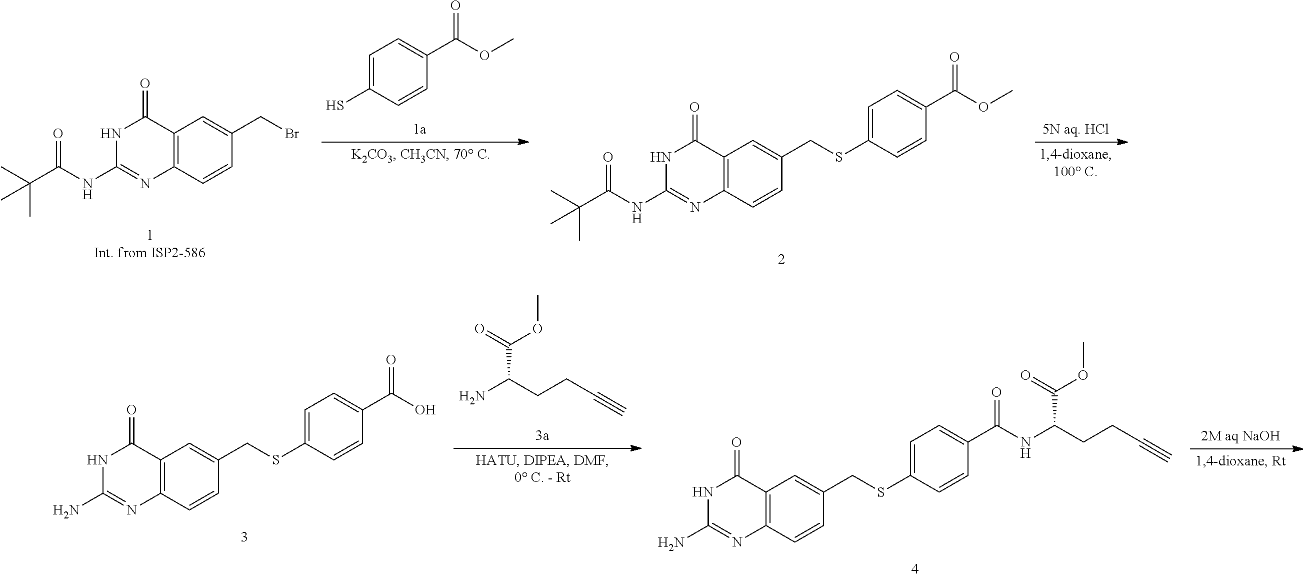

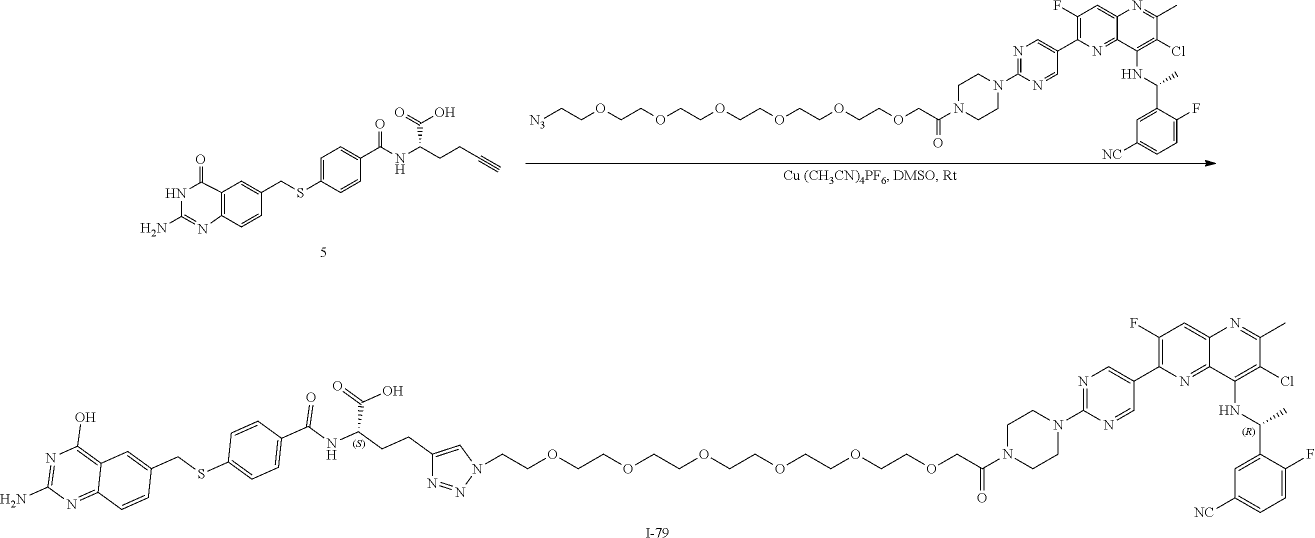

To a stirred solution of N,N-(6-(bromomethyl)-4-oxo-3,4-dihydroquinazolin-2-yl)pivalamide (1, 1.0 g, 1.0 eq., 2.37 mmol) in acetonitrile (10 mL), potassium carbonate (0.908 g, 2.5 eq., 6.57 mmol), and methyl 4-mercaptobenzoate (1a, 0.442 g, 1.0 eq., 2.63 mmol) was added at room temperature and then reaction mixture was stirred at 70° C. for 12 h. After completion, reaction mixture was concentrated under vacuum to give crude residue which was purified by silica gel flash column chromatography using 40-50% ethyl acetate in hexane to afford methyl 4-(((4-oxo-2-pivalamido-3,4-dihydroquinazolin-6-yl)methyl)thio)benzoate (2) as white solid. Yield: 0.70 g, 62.59%; LCMS m/z 426.05 [M+1]⁺.

To a solution of methyl 4-(((4-oxo-2-pivalamido-3,4-dihydroquinazolin-6-yl)methyl)thio)benzoate (2, 0.70 g, 1.0 eq, 1.64 mmol) in 1,4-dioxane (7.0 mL) was added 5N aq. hydrochloric acid (7.0 mL) at room temperature. Reaction mixture was then stirred at 100° C. for 8 h. After completion, reaction mixture was concentrated under vacuum to get crude which was washed with diethyl ether to afford 4-(((2-amino-4-oxo-3,4-dihydroquinazolin-6-yl)methyl)thio)benzoic acid (3) as white solid. Yield: 0.60 g (crude); LCMS m/z 327.95 [M+1]⁺.

To a solution of 4-(((2-amino-4-oxo-3,4-dihydroquinazolin-6-yl)methyl)thio)benzoic acid (3, 0.60 g, 1.0 eq., 1.37 mmol) and methyl (2S)-2-aminohex-5-ynoate (3a, 0.232 g, 1.2 eq., 2.75 mmol) in N,N-dimethylformamide (6.0 mL), were added 1-[Bis(dimethylamino)methylene]-1H-1,2,3-triazolo[4,5-b]pyridinium 3-oxide hexafluorophosphate (HATU)(0.780 g, 1.5 eq., 2.05 mmol) and N, N-Diisopropylethylamine (2.4 mL, 10 eq., 13.7 mmol) at 0° C. Then reaction mixture was stirred at room temperature for 16 h. After completion, water was added to reaction mixture and extracted with ethyl acetate. The organic layer was washed with brine, dried over anhydrous sodium sulfate, filtered and concentrated to get crude which was purified by silica gel flash column chromatography using 0-40% ethyl acetate in hexane as eluent to give methyl (S)-2-(4-(((2-amino-4-oxo-3,4-dihydroquinazolin-6-yl)methyl)thio)benzamido)hex-5-ynoate (4) as brown liquid. Yield: 0.60 g, 72.72%; LCMS m/z 450.90 [M+1]⁺.

To a solution of methyl (S)-2-(4-(((2-amino-4-oxo-3,4-dihydroquinazolin-6-yl)methyl)thio)benzamido)hex-5-ynoate (4, 0.6 g, 1.0 eq., 1.33 mmol) in 1,4-dioxane/water (3:2, v/v, 10 mL), was added sodium hydroxide (0.16 g, 3.0 eq., 4.0 mmol) at 0° C. Reaction mixture was then stirred for 8 h at room temperature, and after completion, reaction mixture was concentrated under vacuum to crude which was dissolved again in water, acidified with aq. hydrochloric acid resulted formation of solid precipitate, which was then filtered out, dried to give (S)-2-(4-(((2-amino-4-oxo-3,4-dihydroquinazolin-6-yl)methyl)thio)benzamido)hex-5-ynoic acid (5) as brown solid. Yield: 0.5 g, Crude; LCMS m/z 437.36 [M+1]⁺.

To a stirred solution of (S)-2-(4-(((2-amino-4-oxo-3,4-dihydroquinazolin-6-yl)methyl)thio)benzamido)hex-5-ynoic acid (5, 0.050 g, 1.0 eq, 0.115 mmol), and (R)-3-(1-((6-(2-(4-(20-azido-3,6,9,12,15,18-hexaoxaicosanoyl)piperazin-1-yl)pyrimidin-5-yl)-3-chloro-7-fluoro-2-methyl-1,5-naphthyridin-4-yl)amino)ethyl)-4-fluorobenzonitrile (5a, 0.10 g, 1.0 eq., 0.115 mmol) in dimethyl sulfoxide (2.0 mL), was added Tetrakis(acetonitrile)copper(I) hexafluorophosphate (0.120 g, 2.8 eq., 0.322 mmol), and stirred at room temperature for 30 minutes. Thereafter, acetic acid (0.5 mL) was added and reaction mixture was purified by prep HPLC (using 23-41% acetonitrile in water with 0.1% TFA). Fractions containing the desired product were combined and lyophilized to dryness to afford (S)-2-(4-(((2-amino-4-hydroxyquinazolin-6-yl)methyl)thio)benzamido)-4-(1-(20-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-20-oxo-3,6,9,12,15,18-hexaoxaicosyl)-1H-1,2,3-triazol-4-yl)butanoic acid (Compound I-79) as an off-white solid. Yield: 0.013 g, 8.65%; LCMS m/z 1304.85 [M+1]⁺. ¹H NMR (400 MHz, DMSO-d₆ with D₂O exchange) δ8.72 (s, 2H), 7.98-7.95 (m, 2H), 7.88 (d, J=4.8 Hz, 1H), 7.78-7.69 (m, 5H), 7.34-7.24 (m, 4H), 6.54 (d, J=6.4 Hz, 1H), 4.40 (t, J=5.2 Hz, 2H), 4.33-4.31 (m, 3H), 4.18 (s, 2H), 3.85-3.82 (m, 4H), 3.71 (t, J=5.2 Hz, 2H), 3.52 (d, J=6.8 Hz, 8H), 3.45-3.37 (m, 18H), 2.65 (s, 3H), 2.12-2.03 (m, 2H), 1.67 (d, J=6.8 Hz, 3H).

### 6.1.33. Example 33: Compound I-80

### 4-(((2-amino-4-hydroxypteridin-6-yl)methyl)amino)-N—((S)-3-((1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)methyl)-1-hydroxy-16-oxo-19-(2H-tetrazol-5-yl)-6,9,12-trioxa-3,15-diazanonadecan-19-yl)benzamide (Compound I-80)

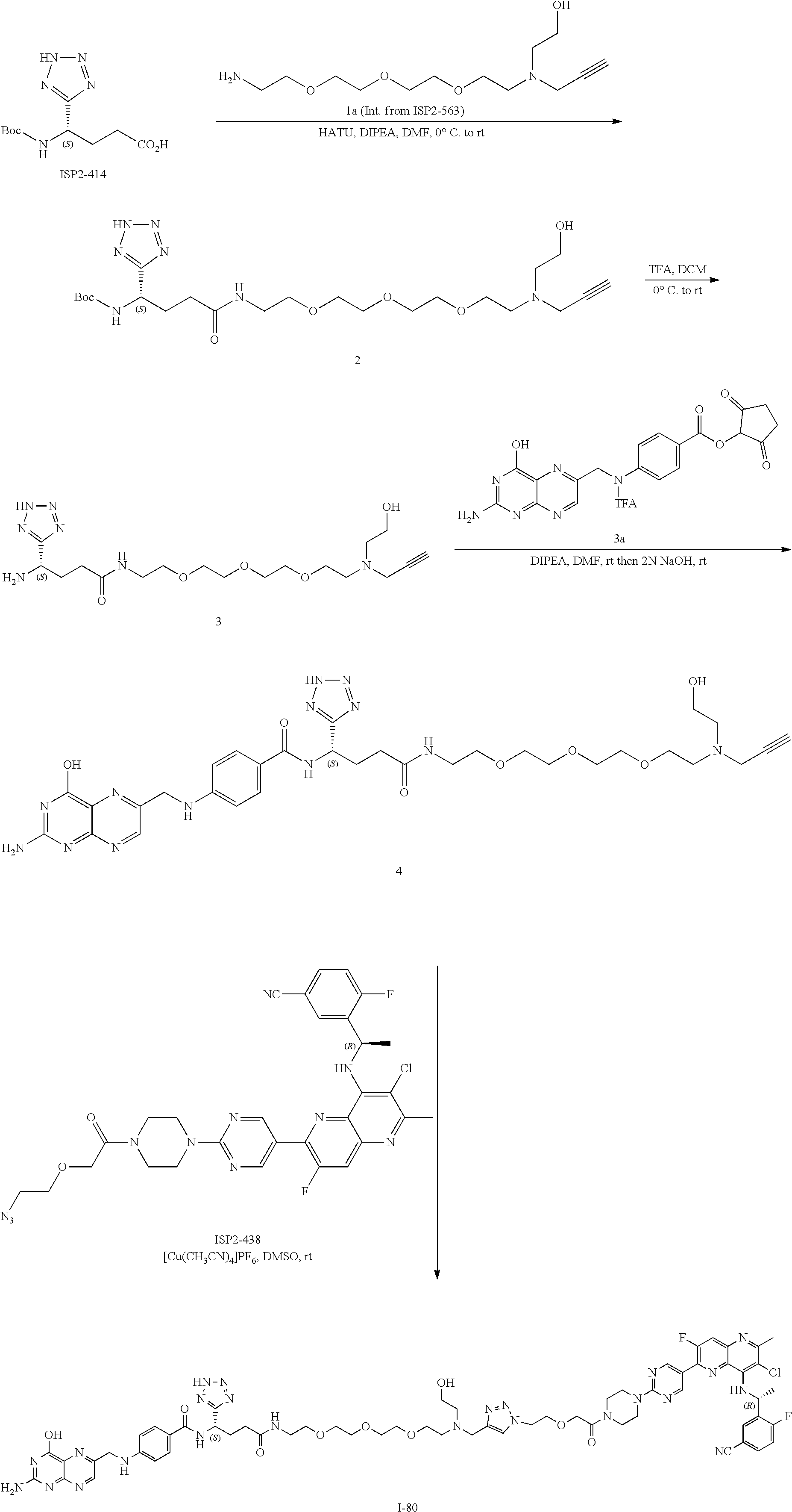

A solution of (S)-4-((tert-butoxycarbonyl)amino)-4-(2H-tetrazol-5-yl)butanoic acid (ISP2-414, 1.0 eq, 0.200 g, 0.737 mmol) and 14-amino-3-(prop-2-yn-1-yl)-6,9,12-trioxa-3-azatetradecan-1-ol (1a, 1.0 eq, 0.202 g, 0.737 mmol) in N,N-dimethylformamide (4 mL) was cooled at 0° C., N,N-diisopropylethylamine (5.0 eq, 0.67 mL, 3.68 mmol) and 1-[bis(dimethylamino)methylene]-1H-1,2,3-triazolo[4,5-b]pyridinium 3-oxid hexafluorophosphate (HATU) (1.5 eq, 0.418 g, 1.10 mmol) were added and reaction mixture was stirred at room temperature for 16 h. After completion, reaction mixture was concentrated to get crude which was purified by column chromatography using silica gel (100-200 mesh) and 0-5% methanol in dichloromethane to afford tert-butyl (S)-(4-(2-hydroxyethyl)-17-oxo-20-(2H-tetrazol-5-yl)-7,10,13-trioxa-4,16-diazaicos-1-yn-20-yl)carbamate (2) as a light yellow viscous liquid. Yield: 0.400 g (Crude); LCMS m/z 528.10 [M+1]⁺.

A solution of tert-butyl (S)-(4-(2-hydroxyethyl)-17-oxo-20-(2H-tetrazol-5-yl)-7,10,13-trioxa-4,16-diazaicos-1-yn-20-yl)carbamate (2, 1.0 eq, 0.400 g, 0.758 mmol) in dichloromethane (2 mL) was cooled at 0° C., trifluoroacetic acid (2 mL) was added and reaction mixture was stirred at room temperature for 3 h. After that, reaction mixture was concentrated. azeotroped with dichloromethane (2-3 times), washed with diethyl ether (2-3 times) and dried to afford (S)-4-amino-N-(12-(2-hydroxyethyl)-3,6,9-trioxa-12-azapentadec-14-yn-1-yl)-4-(2H-tetrazol-5-yl)butanamide (3) as a colourless viscous liquid. Yield: 0.650 g (Crude); LCMS m/z 428.15 [M+1]⁺.

To a suspension of 2,5-dioxopyrrolidin-1-yl 4-(N-((2-amino-4-hydroxypteridin-6-yl)methyl)-2,2,2-trifluoroacetamido)benzoate (3a, 1.0 eq, 0.200 g, 0.395 mmol) and (S)-4-amino-N-(12-(2-hydroxyethyl)-3,6,9-trioxa-12-azapentadec-14-yn-1-yl)-4-(2H-tetrazol-5-yl)butanamide (3, 4.0 eq, 0.675 g, 1.58 mmol) in N,N-dimethylformamide (4 mL) N,N-diisopropylethylamine (5.0 eq, 0.36 mL, 1.97 mmol) was added and reaction mixture was stirred at room temperature for 16 h. After that, 2N aqueous sodium hydroxide solution (2 mL) was added and reaction mixture was stirred at room temperature for 1 h. After completion, reaction mixture was directly purified by prep HPLC (30-55% acetonitrile in water with 0.05% trifluoroacetic acid). Fractions containing the desired compound were combined and lyophilized to afford (S)-4-(((2-amino-4-hydroxypteridin-6-yl)methyl)amino)-N-(4-(2-hydroxyethyl)-17-oxo-20-(2H-tetrazol-5-yl)-7,10,13-trioxa-4,16-diazaicos-1-yn-20-yl)benzamide (4) as a light yellow solid. Yield: 0.007 g, 2.5%; LCMS m/z 721.90 [M+1]⁺.

### Synthesis of 4-(((2-amino-4-hydroxypteridin-6-yl)methyl)amino)-N—((S)-3-((1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)methyl)-1-hydroxy-16-oxo-19-(2H-tetrazol-5-yl)-6,9,12-trioxa-3,15-diazanonadecan-19-yl)benzamide (Cpd. No. ISP2-564)

To a solution of (S)-4-(((2-amino-4-hydroxypteridin-6-yl)methyl)amino)-N-(4-(2-hydroxyethyl)-17-oxo-20-(2H-tetrazol-5-yl)-7,10,13-trioxa-4,16-diazaicos-1-yn-20-yl)benzamide (4, 1.0 eq, 0.007 g, 0.009 mmol) and (R)-3-(1-((6-(2-(4-(2-(2-azidoethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (ISP2-438, 1.0 eq, 0.006 g, 0.009 mmol) in dimethyl sulfoxide (0.5 mL), tetrakis(acetonitrile)copper(I) hexafluorophosphate (1.5 eq, 0.005 g, 0.014 mmol) was added and reaction mixture was stirred at room temperature for 1 h. After completion, reaction mixture was quenched with acetic acid (0.1 mL) and purified by prep HPLC (20-40% acetonitrile in water with 0.05% trifluoroacetic acid). Fractions containing the desired compound were combined and lyophilized to afford 4-(((2-amino-4-hydroxypteridin-6-yl)methyl)amino)-N—((S)-3-((1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)methyl)-1-hydroxy-16-oxo-19-(2H-tetrazol-5-yl)-6,9,12-trioxa-3,15-diazanonadecan-19-yl)benzamide (Compound I-80) as a yellow solid. Yield: 0.0026 g, 19.54%; LCMS m/z 685.54 [(M/2)+1]⁺; ¹H-NMR (400 MHz, DMSO-d₆ with D₂O exchange) δ8.84 (s, 2H), 8.59 (s, 1H), 7.99 (d, J=7.2 Hz, 1H), 7.95 (s, 1H), 7.92-7.88 (m, 1H), 7.75-7.68 (m, 1H), 7.61 (d, J=8.4 Hz, 2H), 7.27 (t, J=9.6 Hz, 1H), 6.60 (d, J=8.8 Hz, 2H), 6.35-3.630 (m, 1H), 4.53-4.51 (m, 2H), 4.43 (s, 2H), 4.19 (s, 2H), 3.53-3.51 (m, 3H), 3.48-3.39 (m, 17H), 3.35-3.28 (m, 6H), 3.11-3.06 (m, 5H), 2.57 (s, 3H), 2.05-2.02 (m, 2H), 1.63 (d, J=7.2 Hz, 3H), 1.19-1.15 (m, 4H).

### 6.1.34. Example 34: Compound I-81

### (S)-2-(4-(((2-amino-4-hydroxypteridin-6-yl)methyl)amino)benzamido)-4-(1-(20-(6-(5-(7-chloro-8-(((R)-1-(5-cyano-2-fluorophenyl)ethyl)amino)-3-fluoro-6-methyl-1,5-naphthyridin-2-yl)pyrimidin-2-yl)-2,6-diazaspiro[3.3]heptan-2-yl)-20-oxo-3,6,9,12,15,18-hexaoxaicosyl)-1H-1,2,3-triazol-4-yl)butanoic acid (Compound I-81)

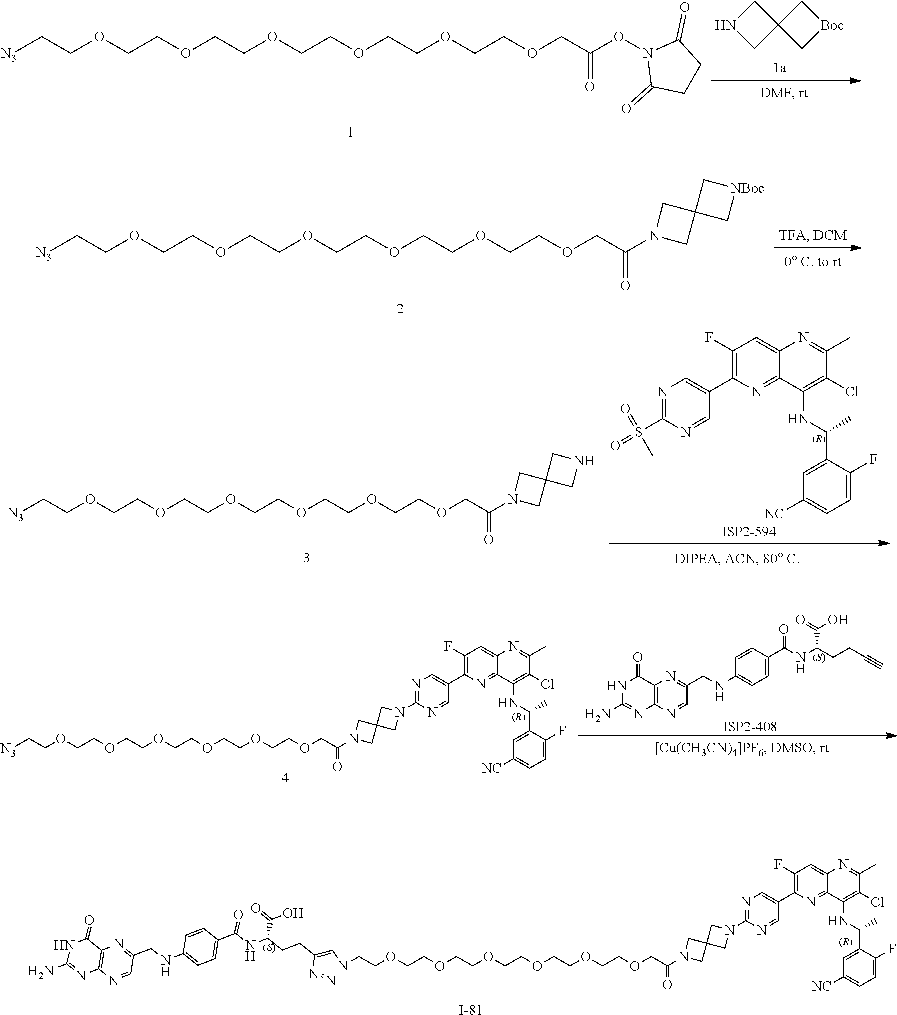

A solution of 2,5-dioxopyrrolidin-1-yl 20-azido-3,6,9,12,15,18-hexaoxaicosanoate (1, 1.0 eq, 0.100 g, 0.216 mmol) and tert-butyl 2,6-diazaspiro[3.3]heptane-2-carboxylate (1a, 1.0 eq, 0.042 g, 0.216 mmol) in N,N-dimethylformamide (1 mL) was stirred at room temperature for 16 h. After completion, reaction mixture was concentrated to get crude product which was purified by column chromatography using silica gel (100-200 mesh) and 0-20% methanol in dichloromethane to afford tert-butyl 6-(20-azido-3,6,9,12,15,18-hexaoxaicosanoyl)-2,6-diazaspiro[3.3]heptane-2-carboxylate (2) as a brown viscous syrup. Yield: 0.080 g, 67.8%: LCMS m/z 546.10 [M+1]⁺.

A solution of tert-butyl 6-(20-azido-3,6,9,12,15,18-hexaoxaicosanoyl)-2,6-diazaspiro[3.3]heptane-2-carboxylate (2, 1.0 eq, 0.080 g, 0.147 mmol) in dichloromethane (0.5 mL) was cooled at 0° C., trifluoroacetic acid (0.5 mL) was added and reaction mixture was stirred at room temperature for 3 h. After completion, reaction mixture was concentrated. azeotroped with dichloromethane (2-3 times), washed with diethyl ether (2-3 times) and dried to afford 20-azido-1-(2,6-diazaspiro[3.3]heptan-2-yl)-3,6,9,12,15,18-hexaoxaicosan-1-one (3) as a colorless viscous liquid. Yield: 0.080 g (Crude); LCMS m/z 446.15 [M+1]⁺.

A solution of (R)-3-(1-((3-chloro-7-fluoro-2-methyl-6-(2-(methylsulfonyl)pyrimidin-5-yl)-1,5-naphthyridin-4-yl)amino)ethyl)-4-fluorobenzonitrile (ISP2-594, 1.0 eq, 0.075 g, 0.146 mmol), 20-azido-1-(2,6-diazaspiro[3.3]heptan-2-yl)-3,6,9,12,15,18-hexaoxaicosan-1-one (3, 2.0 eq, 0.130 g, 0.291 mmol) and N,N-diisopropylethylamine (3.0 eq, 0.08 mL, 0.437 mmol) in acetonitrile (2 mL) was heated at 80° C. for 2 h. After completion, reaction mixture was cooled, concentrated to get crude which was purified by column chromatography using silica gel (100-200 mesh) and 0-70% ethyl acetate in hexane and lyophilized to afford (R)-3-(1-((6-(2-(6-(20-azido-3,6,9,12,15,18-hexaoxaicosanoyl)-2,6-diazaspiro[3.3]heptan-2-yl)pyrimidin-5-yl)-3-chloro-7-fluoro-2-methyl-1,5-naphthyridin-4-yl)amino)ethyl)-4-fluorobenzonitrile (4) as a light yellow sticky solid. Yield: 0.070 g, 54.59%: LCMS m/z 880.10 [M+1]⁺.

### Synthesis of (S)-2-(4-(((2-amino-4-hydroxypteridin-6-yl)methyl)amino)benzamido)-4-(1-(20-(6-(5-(7-chloro-8-(((R)-1-(5-cyano-2-fluorophenyl)ethyl)amino)-3-fluoro-6-methyl-1,5-naphthyridin-2-yl)pyrimidin-2-yl)-2,6-diazaspiro[3.3]heptan-2-yl)-20-oxo-3,6,9,12,15,18-hexaoxaicosyl)-1H-1,2,3-triazol-4-yl)butanoic acid (Cpd. No. ISP2-584)

To a solution of (R)-3-(1-((6-(2-(6-(20-azido-3,6,9,12,15,18-hexaoxaicosanoyl)-2,6-diazaspiro[3.3]heptan-2-yl)pyrimidin-5-yl)-3-chloro-7-fluoro-2-methyl-1,5-naphthyridin-4-yl)amino)ethyl)-4-fluorobenzonitrile (4, 1.0 eq, 0.070 g, 0.079 mmol) and (S)-2-(4-(((2-amino-4-oxo-3,4-dihydropteridin-6-yl)methyl)amino)benzamido)hex-5-ynoic acid (ISP2-408, 1.0 eq, 0.033 g, 0.079 mmol) in dimethyl sulfoxide (1.5 mL), tetrakis(acetonitrile)copper(I) hexafluorophosphate (1.5 eq, 0.044 g, 0.119 mmol) was added and reaction mixture was stirred at room temperature for 1 h. After completion, reaction mixture was quenched with acetic acid (0.2 mL) and purified by prep HPLC (15-30% acetonitrile in water with 0.05% trifluoroacetic acid). Fractions containing the desired compound were combined and lyophilized to afford (S)-2-(4-(((2-amino-4-hydroxypteridin-6-yl)methyl)amino)benzamido)-4-(1-(20-(6-(5-(7-chloro-8-(((R)-1-(5-cyano-2-fluorophenyl)ethyl)amino)-3-fluoro-6-methyl-1,5-naphthyridin-2-yl)pyrimidin-2-yl)-2,6-diazaspiro[3.3]heptan-2-yl)-20-oxo-3,6,9,12,15,18-hexaoxaicosyl)-1H-1,2,3-triazol-4-yl)butanoic acid (Compound I-81) as a light yellow solid. Yield: 0.036 g, 33.16%; LCMS m/z 651.50 [(M/2)+1]⁺; ¹H NMR (400 MHz, DMSO-d6 with D₂O exchange) δ8.75 (s, 2H), 8.63 (s, 1H), 7.97 (d, J=11.2 Hz, 1H), 7.84 (d, J=6.4 Hz, 1H), 7.69-7.63 (m, 2H), 7.60 (d, J=8.4 Hz, 2H), 7.24 (t, J=8.4 Hz, 1H), 7.64 (d, J=8.8 Hz, 2H), 6.47 (d, J=7.2 Hz, 1H), 4.48 (s, 2H), 4.45-4.35 (m, 4H), 4.28 (s, 4H), 4.20-4.05 (m, 2H), 3.94 (s, 2H), 3.74 (t, J=5.2 Hz, 2H), 3.49-3.42 (m, 16H), 2.64 (s, 3H), 2.11-2.06 (m, 2H), 1.68 (d, J=6.4 Hz, 3H).

### 6.1.35. Example 35: Compound I-82

### (S)-2-(4-(2-(2-amino-4-hydroxyquinazolin-6-yl)ethyl)benzamido)-4-(1-(20-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-20-oxo-3,6,9,12,15,18-hexaoxaicosyl)-1H-1,2,3-triazol-4-yl)butanoic acid (I-82)

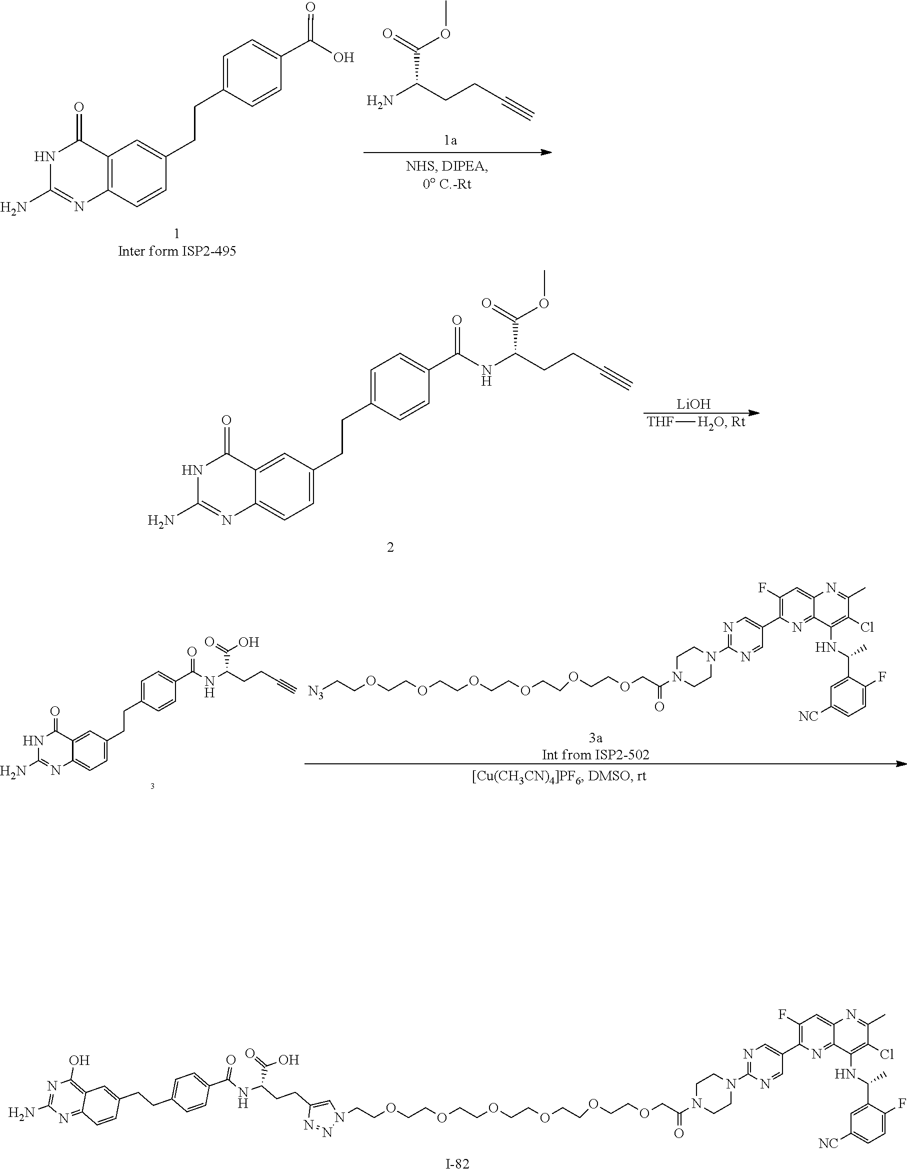

To a stirred solution of 4-(2-(2-amino-4-oxo-3,4-dihydroquinazolin-6-yl)ethyl)benzoic acid (0.20 g, 1.0 eq., 0.647 mmol) in N,N dimethylformamide (2 mL), N-hydroxysuccinimide (0.369 g, 1.5 eq., 0.970 mmol) followed by N,N-diisopropylethylamine (0.565 mL, 5.0 eq., 3.23 mmol) were added at 0° C., and then reaction mixture was stirred at room temperature for 2 h. Thereafter, methyl (S)-2-aminohex-5-ynoate (0.119 g, 1.3 eq., 0.841 mmol) was added to the reaction mixture, and again stirred for further 3 h. After completion, reaction mixture was concentrated under vacuum to get crude product which was purified by silica gel flash column chromatography using 5-8% methanol in dichloromethane to give methyl (S)-2-(4-(2-(2-amino-4-oxo-3,4-dihydroquinazolin-6-yl)ethyl)benzamido)hex-5-ynoate (2) as pale yellow solid. Yield: 0.150 g, 53.0%; LCMS m/z 431.05 [M−1]⁻.

To a stirred solution of methyl (S)-2-(4-(2-(2-amino-4-oxo-3,4-dihydroquinazolin-6-yl)ethyl)benzamido)hex-5-ynoate (2, 0.150 g, 1.0 eq., 0.347 mmol) in tetrahydrofuran:water (3 mL, 1:1, v/v) was added lithium hydroxide monohydrate (0.017 g, 2.0 eq., 0.694 mmol) at room temperature. The reaction mixture was stirred for 5 h and then neutralized with 1N aq. HCl to give white precipitate. The reaction mixture was then centrifuged to give white solid which was washed further with water, acetonitrile and diethyl ether sequentially to give crude as white solid which was used without further purification for next step. Yield: 0.075 g (Crude); LCMS m/z 419.05 [M+1].

To a solution of (S)-2-(4-(2-(2-amino-4-oxo-3,4-dihydroquinazolin-6-yl)ethyl)benzamido)hex-5-ynoic acid (0.025 g, 1.0 eq, 0.0597 mmol) and (R)-3-(1-((6-(2-(4-(20-azido-3,6,9,12,15,18-hexaoxaicosanoyl)piperazin-1-yl)pyrimidin-5-yl)-3-chloro-7-fluoro-2-methyl-1,5-naphthyridin-4-yl)amino)ethyl)-4-fluorobenzonitrile (0.0519 g, 1.0 eq, 0.0597 mmol) in dimethyl sulfoxide (2 ml) was added tetrakis(acetonitrile)copper(I) hexafluorophosphate (0.0334 g, 1.5 eq., 0.090 mmol). The reaction mixture was stirred for 1 h. The reaction mixture was quenched with acetic acid (0.2 ml) and purified by prep-HPLC (using 50-65% acetonitrile with 0.1% TFA). The desired fractions were collected and lyophilized to give (S)-2-(4-(2-(2-amino-4-hydroxyquinazolin-6-yl)ethyl)benzamido)-4-(1-(20-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-20-oxo-3,6,9,12,15,18-hexaoxaicosyl)-1H-1,2,3-triazol-4-yl)butanoic acid (Compound I-82) as a yellow solid. Yield: 0.016 g, 20.81%; LCMS: 1308.35 [M+Na]⁺. ¹H NMR (400 MHz, DMSO-d₆ with H₂O exchange) δ8.78 (s, 2H), 7.98 (d, J=11.2 Hz, 1H), 7.90 (d, J=6.8 Hz, 1H), 7.77-7.72 (m, 5H), 7.61 (dd, J=1.6, 8.4 Hz, 1H), 7.31-7.25 (m, 4H), 6.45 (d, J=6.8 Hz, 1H), 4.41 (t, J=4.8 Hz, 2H), 4.33 (dd, J=4.8, 9.2 Hz, 1H), 4.18 (s, 2H), 3.72 (t, J=4.8 Hz, 2H), 3.54-3.52 (m, 8H), 3.46-3.39 (m, 17H), 2.95-2.89 (m, 4H), 2.72-2.68 (m, 1H), 2.62 (s, 3H), 2.14-2.06 (m, 2H), 1.65 (d, J=6.8 Hz, 3H).

### 6.1.36. Example 36: Compound I-83

### (S)-2-(4-(((2-amino-4-oxo-3,4-dihydropteridin-6-yl)methyl)(methyl)amino)benzamido)-4-(1-(20-(4-(5-(1-(2-(difluoromethoxy)benzyl)-2-((3-(2-oxopyrrolidin-1-yl)phenoxy)methyl)-1H-benzo[d]imidazol-6-yl)pyrimidin-2-yl)piperazin-1-yl)-20-oxo-3,6,9,12,15,18-hexaoxaicosyl)-1H-1,2,3-triazol-4-yl)butanoic acid (Compound I-83)

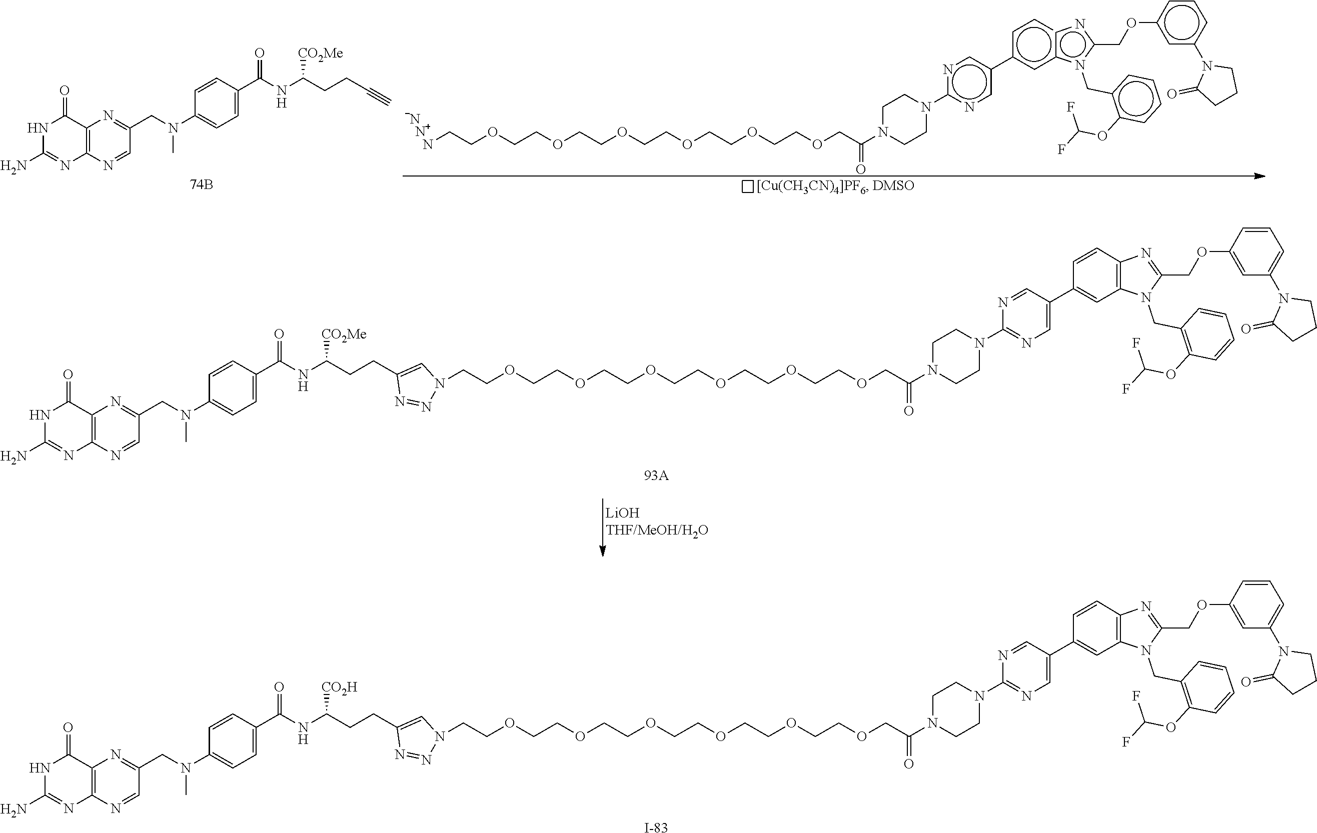

To a mixture of methyl (2S)-2-[[4-[(2-amino-4-oxo-3H-pteridin-6-yl)methyl-methyl-amino]benzoyl]amino]hex-5-ynoate (74B, 1.00 eq, 14.2 mg, 0.032 mmol) and 1-[3-[[6-[2-[4-[2-[2-[2-[2-[2-[2-(2-azidoethoxy)ethoxy]ethoxy]ethoxy]ethoxy]ethoxy]acetyl]piperazin-1-yl]pyrimidin-5-yl]-1-[[2-(difluoromethoxy)phenyl]methyl]benzimidazol-2-yl]methoxy]phenyl]pyrrolidin-2-one (1.00 eq, 30.7 mg, 0.032 mmol) in DMSO (0.7 mL) was added Tetrakis(acetonitrile)copper(I) hexafluorophosphate (0.5 eq, 5.9 mg, 0.016 mmol). The mixture was stirred at room temperature for 1 h. More Tetrakis(acetonitrile)copper(I) hexafluorophosphate (5.7 mg) was added and the mixture was stirred at room temperature for 1 h. More Tetrakis(acetonitrile)copper(I) hexafluorophosphate (5.1 mg) was added and the mixture was stirred at room temperature overnight. Then SiliaMetS Imidazole resin (210 mg, 1.11 mmol/g) was added. The mixture was stirred room temperature for 1 h and filtered. The filtrate was purified by prep. HPLC (20-55% MeCN/H₂O with 0.1% formic acid) to give 93A as a yellow solid (15.7 mg, yield: 35%). LCMS m/z 1422.7 [m+H]⁺.

To a mixture of methyl (2S)-2-[[4-[(2-amino-4-oxo-3H-pteridin-6-yl)methyl-methyl-amino]benzoyl]amino]-4-[1-[2-[2-[2-[2-[2-[2-[2-[4-[5-[3-[[2-(difluoromethoxy)phenyl]methyl]-2-[[3-(2-oxopyrrolidin-1-yl)phenoxy]methyl]benzimidazol-5-yl]pyrimidin-2-yl]piperazin-1-yl]-2-oxo-ethoxy]ethoxy]ethoxy]ethoxy]ethoxy]ethoxy]ethyl]triazol-4-yl]butanoate (93A, 1.00 eq, 15.7 mg, 0.0110 mmol) in THF (0.15 mL) and MeOH (0.1 mL) was added 1N LiOH (22 mL, 0.022 mmol) and the mixture was stirred at room temperature for 1 h. More 1N LiOH (11 mL, 0.011 mmol) was added and the mixture was stirred at room temperature for 30 minutes. More 1N LiOH (2.2 mL, 0.0022 mmol) was added and the mixture was stirred at room temperature for 30 minutes. formic acid (4 mL) was added. The mixture was concentrated to remove organic solvent and purified by prep. HPLC (20-55% MeCN/H₂O with 0.1% formic acid) to give Compound I-83 as a yellow solid (9.6 mg, yield: 62%). LCMS m/z 1408.5 [M+H]⁺.

### 6.1.37. Example 37: Compound I-84

### (S)-2-(4-(((2-amino-4-hydroxypteridin-6-yl)methyl)amino)benzamido)-5-(9-(2-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethoxy)ethyl)-3,9-diazaspiro[5.5]undecan-3-yl)-5-oxopentanoic acid (Compound I-84)

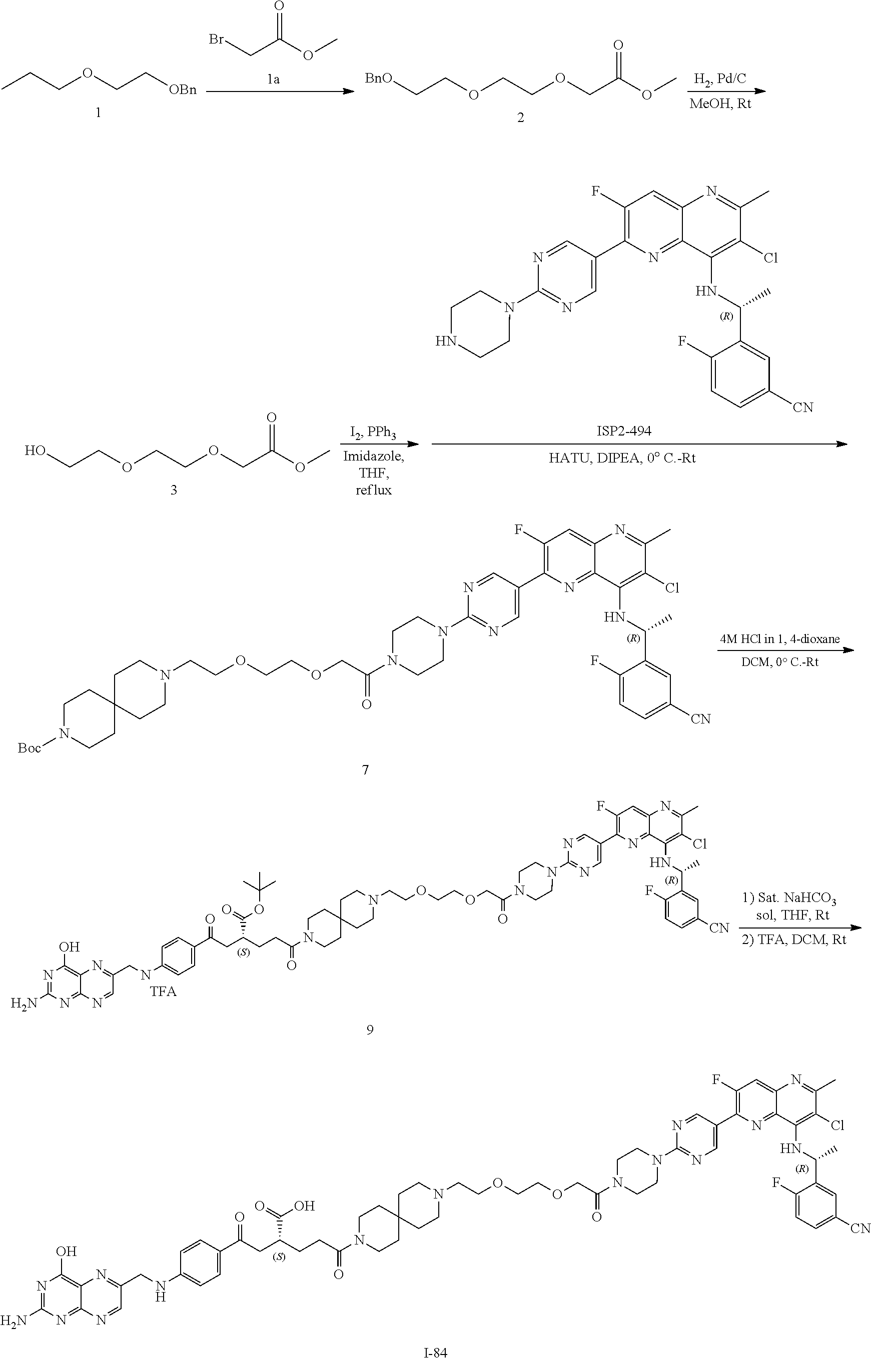

To a stirred solution of 2-(2-(benzyloxy)ethoxy)ethan-1-ol (2.0 g, 1.0 eq, 10.2 mmol) in tetrahydrofuran (25 0 mL) was added sodium hydride (60%, 0.611 g, 1.5 eq, 15.3 mmol) at 0° C. The reaction mixture was stirred at the same temperature for 10 minutes, and then a solution of methyl 2-bromoacetate (1.87 g, 1.2 eq, 12.2 mmol) in tetrahydrofuran (5.0 mL) was added to the reaction mixture slowly at 0° C., and stirred at room temperature for 16 h. After completion, reaction mixture was quenched with water and extracted with ethyl acetate. The organic layer was dried over anhydrous sodium sulfate, filtered and concentrated under reduced pressure to obtain crude product which was purified by silica gel column chromatography using 10-100% ethyl acetate in hexane to afford methyl 2-(2-(2-(benzyloxy)ethoxy)ethoxy)acetate (2) as a light yellow syrup Yield: 1.20 g, 43.89%; LCMS m/z 269.3 [M+1]⁺.

To a solution of methyl 2-(2-(2-(benzyloxy)ethoxy)ethoxy)acetate (2, 1.2 g, 1.0 eq, 4.47 mmol) in methanol (10 mL), 10% palladium on carbon (0.700 g) was added and reaction mixture was stirred at room temperature under hydrogen gas atmosphere for 3 h. After completion, reaction mixture was filtered through celite bed and bed was washed with methanol (2 times). The filtrate was concentrated and dried to give methyl 2-(2-(2-hydroxyethoxy)ethoxy)acetate (3) as a colorless viscous liquid. Yield: 0.720 g (Crude); LCMS m/z 179.10 [M+1]⁺.

To a solution of methyl 2-(2-(2-hydroxyethoxy)ethoxy)acetate (3, 0.720 g, 1.0 eq, 4.04 mmol) in tetrahydrofuran (30 mL), were added triphenylphosphine(1.27 g, 1.2 eq, 4.85 mmol), imidazole (0.440 g, 1.6 eq, 6.470 mmol) and molecular iodine (1.2 eq, 1.23 g, 4.85 mmol) at room temperature. Thereafter, reaction mixture was refluxed for 1 h. After completion, reaction mixture was cooled, water was added and extracted with ethyl acetate. The organic layer was dried over anhydrous sodium sulfate, filtered and concentrated to get crude which was purified by silica gel column chromatography using 0-25% ethyl acetate in hexane to afford methyl 2-(2-(2-iodoethoxy)ethoxy)acetate (4) as a white solid. Yield: 0.750 g, 64.43%; LCMS m/z 289.1 [M+1]⁺.

To a stirred the solution of methyl 2-(2-(2-iodoethoxy)ethoxy)acetate (4, 0.75 g, 1.0 eq., 2.6 mmol) in acetonitrile (7 mL), were added tert-butyl 3,9-diazaspiro[5.5]undecane-3-carboxylate (0.795 g, 1.2 eq, 3.12 mmol) and potassium carbonate (1.08 g, 3.0 eq., 7.81 mmol) and then the suspension was stirred at 60° C. for overnight. After completion, reaction mixture was cooled, water was added and extracted with ethyl acetate. The organic layer was dried over anhydrous sodium sulfate, filtered and concentrated to get crude which was purified by silica gel column chromatography using 0-60% ethyl acetate in hexane to afford tert-butyl 9-(2-(2-(2-methoxy-2-oxoethoxy)ethoxy)ethyl)-3,9-diazaspiro[5.5]undecane-3-carboxylate (5) as viscous liquid. Yield: 0.550 g, 50.96%. LCMS m/z 415.5 [M+1]⁺.

To a solution of tert-butyl 9-(2-(2-(2-methoxy-2-oxoethoxy)ethoxy)ethyl)-3,9-diazaspiro[5.5]undecane-3-carboxylate (5, 0.550 g, 1.0 eq, 1.33 mmol) in tetrahydrofuran:methanol:water (10 mL, 3:2:1, v/v), lithium hydroxide monohydrate (0.167 g, 3.0 eq, 3.98 mmol) was added, and reaction mixture was stirred at room temperature for 16 h. After completion, reaction mixture was concentrated, and water was added. Then, aqueous layer was neutralized with 1N hydrochloric acid and extracted with 10% methanol in dichloromethane. The organic layer was dried over anhydrous sodium sulfate, filtered, concentrated and dried to afford 2-(2-(2-(9-(tert-butoxycarbonyl)-3,9-diazaspiro[5.5]undecan-3-yl)ethoxy)ethoxy)acetic acid (6) as a colorless oil. Yield: 0.520 g (Crude); LCMS m/z 401.50 [M+1]⁺.

To a stirred solution of 2-(2-(2-(9-(tert-butoxycarbonyl)-3,9-diazaspiro[5.5]undecan-3-yl)ethoxy)ethoxy)acetic acid (6, 0.200 g, 1.0 eq, 0.499 mmol) and (R)-3-(1-((3-chloro-7-fluoro-2-methyl-6-(2-(piperazin-1-yl)pyrimidin-5-yl)-1,5-naphthyridin-4-yl)amino)ethyl)-4-fluorobenzonitrile (ISP2-494, 0.286 g, 1.10 eq., 0.549 mmol) in dimethylformamide (6 mL) were added hexafluoro-λ⁵-phosphanuide 1-[bis(dimethylamino)methylidene]-1H-1λ⁵-[1,2,3]triazolo[4,5-b]pyridin-3-ium-1-ylium-3-olate (HATU) (0.247 g, 1.3 eq., 0.649 mmol) and N, N-diisopropyethylamine (0.262 mL, 3 eq., 1.5 mmol) at 0° C. Then reaction mixture was stirred at room temperature for overnight. After completion, reaction mixture was diluted with ethyl acetate and the washed with water. Aqueous layer was extracted twice with ethyl acetate. The combined organic phase was washed with brine, dried over anhydrous sodium sulfate, filtered and the filtrate was concentrated under reduce pressure to obtain crude which was purified by silica gel flash column chromatography using 0-10% methanol in dichloromethane as eluent to afford tert-butyl (R)-9-(2-(2-(2-(4-(5-(7-chloro-8-((1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethoxy)ethyl)-3,9-diazaspiro[5.5]undecane-3-carboxylate (7) as a light yellow syrup. Yield: 0.220 g, 48.76%. LCMS m/z 903.49 [M+1].

To a solution of tert-butyl (R)-9-(2-(2-(2-(4-(5-(7-chloro-8-((1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethoxy)ethyl)-3,9-diazaspiro[5.5]undecane-3-carboxylate (7, 0.200 g, 1.0 eq, 0.221 mmol) in dichloromethane (1 mL) was cooled to 0° C., 4N HCl in 1,4-dioxane (3 mL) was added and reaction mixture was stirred at room temperature for 3 h. After completion, reaction mixture was concentrated, azeotroped with dichloromethane (2-3 times), washed with diethyl ether (2-3 times) and dried to afford (R)-3-(1-((6-(2-(4-(2-(2-(2-(3,9-diazaspiro[5.5]undecan-3-yl)ethoxy)ethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (8) as a yellow solid. Yield: 0.220 g (Crude); LCMS m/z 803.45 [M+1].

To a stirred solution of (R)-3-(1-((6-(2-(4-(2-(2-(2-(3,9-diazaspiro[5.5]undecan-3-yl)ethoxy)ethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (8, 0.08 g, 1.0 eq, 0.095 mmol) and (S)-4-(4-(N-((2-amino-4-hydroxypteridin-6-yl)methyl)-2,2,2-trifluoroacetamido)benzamido)-5-(tert-butoxy)-5-oxopentanoic acid (8a, 0.062 g, 1.1 eq, 0.105 mmol) in dimethylformamide (2 mL) were added hexafluoro-λ⁵-phosphanuide 1-[bis(dimethylamino)methylidene]-1H-1λ⁵-[1,2,3]triazolo[4,5-b]pyridin-3-ium-1-ylium-3-olate (HATU) (0.047 g, 1.3 eq., 0.124 mmol) and N, N-diisopropyethylamine (0.049 mL, 3.0 eq., 0.286 mmol) at 0° C., and then reaction mixture was stirred at room temperature for overnight. After completion of reaction, reaction mixture was concentrated under vaccum to get crude tert-butyl (S)-2-(4-(N-((2-amino-4-hydroxypteridin-6-yl)methyl)-2,2,2-trifluoroacetamido)benzamido)-5-(9-(2-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethoxy)ethyl)-3,9-diazaspiro[5.5]undecan-3-yl)-5-oxopentanoate (9) as yellow solid which was use as such for next step. Yield: 0.20 g (Crude); LCMS m/z 689.8 [M/2+1]⁺.

To a stirred suspension of tert-butyl (S)-2-(4-(N-((2-amino-4-hydroxypteridin-6-yl)methyl)-2,2,2-trifluoroacetamido)benzamido)-5-(9-(2-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethoxy)ethyl)-3,9-diazaspiro[5.5]undecan-3-yl)-5-oxopentanoate (9, 0.2 g, 0.145 mmol) in tetrahydrofuran (2 mL) was added sat. aq. Sodium bicarbonate solution and stirred for 12 h at room temperature. Thereafter, volatiles were evaporated out to give crude. To this, dichloromethane (3 mL) and trifluoroacetic acid (5 mL) were added and stirred at room temperature for 4 h. After completion, reaction mixture was purified by prep HPLC (using 25-50% acetonitrile in water with 0.1% TFA). Fractions containing the desired compound were combined and lyophilized to afford (S)-2-(4-(((2-amino-4-hydroxypteridin-6-yl)methyl)amino)benzamido)-5-(9-(2-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethoxy)ethyl)-3,9-diazaspiro[5.5]undecan-3-yl)-5-oxopentanoic acid (Compound I-84) as yellow solid. Yield: 0.009 g, 9.69%; LCMS m/z 1226.65 [M+1]⁺. ¹H NMR (400 MHz, DMSO-d₆ with H₂O exchange) δ8.86 (s, 2H), 8.64 (s, 8.04 (d, J=11.2 Hz, 1H), 7.97 (d, J=6.4 Hz, 1H), 7.62-7.58 (m, 1H), 7.61 (d, J=8.4 Hz, 2H), 7.31 (t, J=8.8 Hz, 1H), 6.63 (d, J=8.8 Hz, 2H), 6.44-6.43 (m, 1H), 4.47 (s, 2H), 4.31-4.29 (m, 1H), 4.26 (s, 2H), 3.89-3.87 (m, 4H), 3.77-3.75 (m, 2H), 3.62 (m, 4H), 3.42-3.27 (m, 8H), 3.08-3.04 (m, 2H), 2.64 (s, 3H), 2.39-2.37 (m, 2H), 2.03-1.90 (m, 2H), 1.80-1.75 (m, 2H), 1.67 (d, J=6.8 Hz, 3H), 1.59-1.42 (m, 4H), 1.59-1.42 (m, 4H).

### 6.1.38. Example 38: Compounds I-85 to I-89

### (S)-2-(4-(((2-amino-4-oxo-3,4-dihydroquinazolin-6-yl)methyl)(methyl)amino)benzamido)-4-(1-(20-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-20-oxo-3,6,9,12,15,18-hexaoxaicosyl)-1H-1,2,3-triazol-4-yl)butanoic acid (Compound I-85)

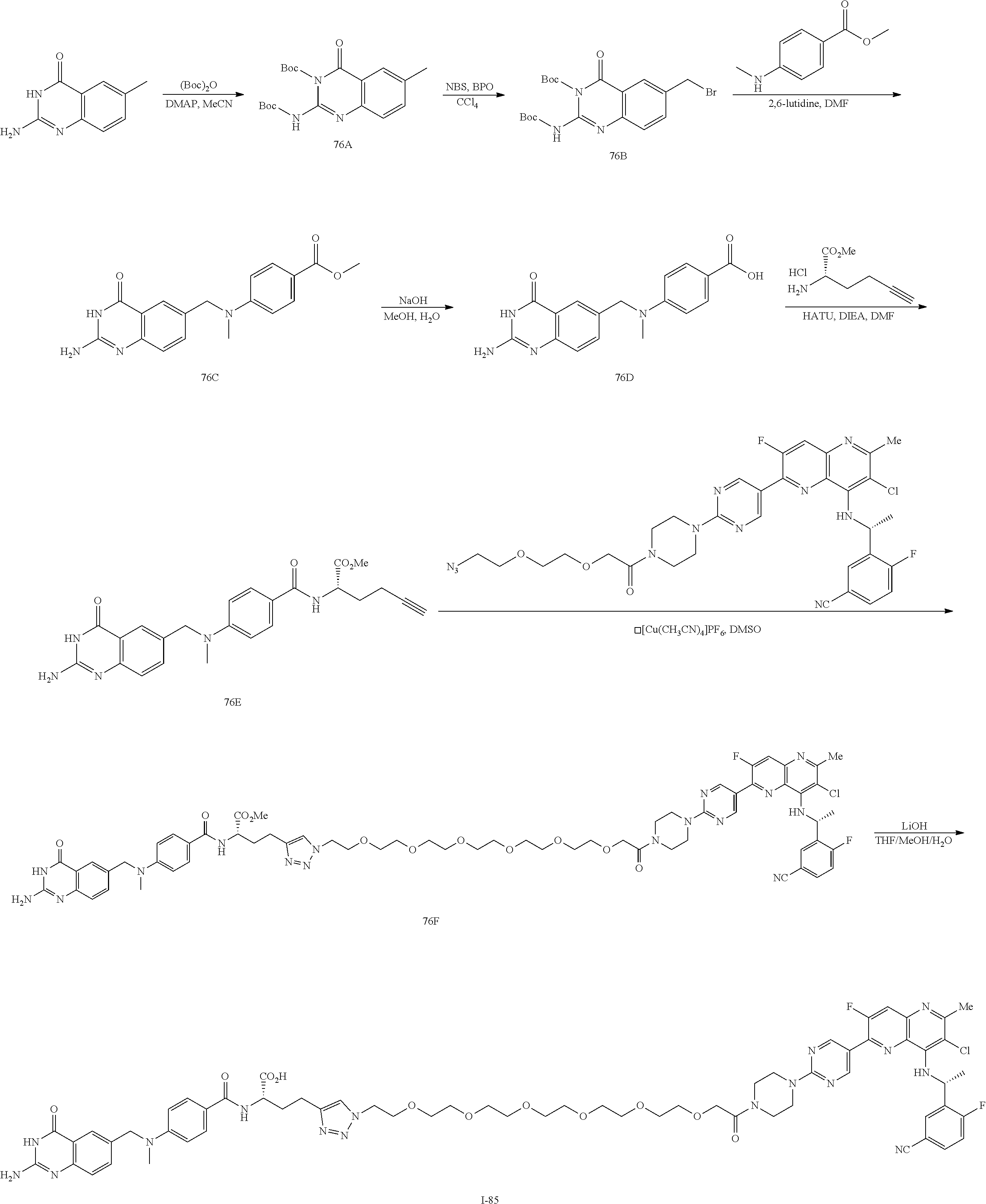

To a mixture of 2-amino-6-methyl-3H-quinazolin-4-one (1.00 eq, 250 mg, 1.43 mmol) in MeCN (10 mL) were added di-tert-buyl dicarbonate (4.00 eq, 1.25 g, 5.71 mmol) and 4-(Dimethylamino)pyridine (0.200 eq, 34.9 mg, 0.29 mmol). The mixture was stirred at room temperature overnight. The mixture was concentrated and purified by column (0-20% EtOAc/hexane) to give 76A as a white solid (267.7 mg, yield: 50%). LCMS m/z 376.3 [M+H]f.

To a mixture of tert-butyl N-tert-butoxycarbonyl-N-(6-methyl-4-oxo-3H-quinazolin-2-yl)carbamate (76A, 1.00 eq, 383 mg, 1.02 mmol) in carbon tetrachloride (6 mL) were added N-Bromosuccinimide (1.20 eq, 218 mg, 1.22 mmol) and Benzoyl Peroxide (0.027 eq, 6.7 mg, 0.028 mmol). The mixture was stirred at 80° C. for 8 h. The mixture was filtered, and the filtrate was concentrated and purified by column (0-5% EtOAc/DCM) to give 76B as a white solid (260 mg, yield: 56%).

To a mixture of tert-butyl N-[6-(bromomethyl)-4-oxo-3H-quinazolin-2-yl]-N-tert-butoxycarbonyl-carbamate (76B, 1.00 eq, 94.0 mg, 0.21 mmol) and methyl 4-(methylamino)benzoate (1.00 eq, 34.2 mg, 0.21 mmol) in DMF (0.7 mL) was added 2,6-Lutidine (3.00 eq, 0.072 mL, 0.62 mmol). The mixture was stirred at 50° C. overnight and concentrated. The residue was diluted with DCM (0.5 mL) and TFA (1 mL) was added. The mixture was stirred at room temperature for 1 h, concentrated, and purified by column (0-10% MeOH/DCM) to give 76C as a white solid (66 mg, yield: 94%). LCMS m/z 339.2 [M+H]⁺.

To a mixture of methyl 4-[(2-amino-4-oxo-3H-quinazolin-6-yl)methyl-methyl-amino]benzoate (76C, 1.00 eq, 29.0 mg, 0.086 mmol) in MeOH (0.9 mL) was added 1N NaOH (0.52 mL). The mixture was stirred at 50° C. overnight and concentrated to remove organic solvent. The aqueous solution was acidified with 1N HCl. The precipitate was collected via filtration, washed with water, dried under high vacuum to give 76D as a yellow solid (21.5 mg, yield: 77%). LCMS m/z 325.1 [M+H]⁺.

To a mixture of 4-[(2-amino-4-oxo-3H-quinazolin-6-yl)methyl-methyl-amino]benzoic acid (76D, 1.00 eq, 21.5 mg, 0.066 mmol) and methyl (2S)-2-aminohex-5-ynoate;hydrochloride (1.20 eq, 14.1 mg, 0.080 mmol) in DMF (0.6 mL) were added DIEA (46 mL, 0.27 mmol) and HATU (30.2 mg, 0.80 mmol). The mixture was stirred at room temperature for 1 h and purified by prep. HPLC (20-60% MeCN/H₂O with 0.1% formic acid) to give 76E as a yellow solid (16.8 mg, yield: 57%). LCMS m/z 448.2 [M+H]⁺.

To a mixture of methyl (2S)-2-[[4-[(2-amino-4-oxo-3H-quinazolin-6-yl)methyl-methyl-amino]benzoyl]amino]hex-5-ynoate (76E, 1.00 eq, 16.8 mg, 0.0375 mmol) and 3-[(1R)-1-[[6-[2-[4-[2-[2-[2-[2-[2-[2-(2-azidoethoxy)ethoxy]ethoxy]ethoxy]ethoxy]ethoxy]acetyl]piperazin-1-yl]pyrimidin-5-yl]-3-chloro-7-fluoro-2-methyl-1,5-naphthyridin-4-yl]amino]ethyl]-4-fluoro-benzonitrile (0.99 eq, 32.2 mg, 0.0371 mmol) in DMSO (0.6 mL) was added Tetrakis(acetonitrile)copper(I) hexafluorophosphate (0.5 eq, 7 mg, 0.019 mmol). The mixture was stirred at room temperature for 1 h. More Tetrakis(acetonitrile)copper(I) hexafluorophosphate (7.8 mg) was added and the mixture was stirred at room temperature for 45 minutes. More Tetrakis(acetonitrile)copper(I) hexafluorophosphate (8.1 mg) was added and the mixture was stirred at room temperature for 45 minutes. Then SiliaMetS Imidazole resin (240 mg, 1.11 mmol/g) was added. The mixture was stirred for 1 h and filtered. The filtrate was purified by prep. HPLC (20-55% MeCN/H₂O with 0.1% formic acid) to give 76F as a yellow solid (17.4 mg, yield: 35%). LCMS m/z 1315.5 [M+H]⁺.

To a mixture of methyl (2S)-2-[[4-[(2-amino-4-oxo-3H-quinazolin-6-yl)methyl-methyl-amino]benzoyl]amino]-4-[1-[2-[2-[2-[2-[2-[2-[2-[4-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]piperazin-1-yl]-2-oxo-ethoxy]ethoxy]ethoxy]ethoxy]ethoxy]ethoxy]ethyl]triazol-4-yl]butanoate (76F, 1.00 eq, 17.4 mg, 0.013 mmol) in THF (0.15 mL) and MeOH (0.1 mL) was added 1N LiOH (26.5 mL, 0.0265 mmol). The mixture was stirred at room temperature for 30 minutes. More 1N LiOH (13.3 mL, 0.0133 mmol) was added and the mixture was stirred at room temperature for 30 minutes. More 1N LiOH (2.6 mL, 0.0026 mmol) was added and the mixture was stirred at room temperature for 30 minutes. The mixture was acidified with formic acid (5 mL) and concentrated to remove organic solvent. The mixture was dissolved in DMSO (1.2 mL) and was purified by prep. HPLC (20-55% MeCN/H₂O with 0.1% formic acid) to give Compound I-85 as a yellow solid (11.8 mg, yield: 68.5%). LCMS m z 1301.6 [M+H]⁺.

Compound I-86 to I-89 were prepared by adapting the general method described above for compound I-85. The structures, names and data for compounds I-86 to I-89 are provided below in Table X:

TABLE X
Data for compounds I-XX to I-XX

| # | Compound structure, name and Data |
|---|---|
| I-86 | 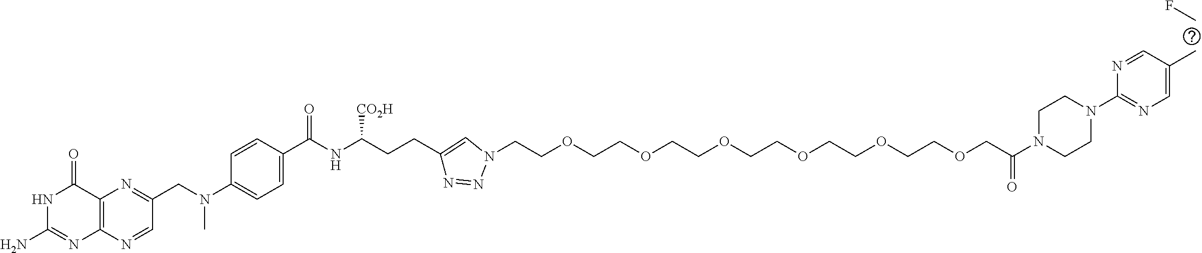 |
| | (S)-2-(4-(((2-amino-4-oxo-3,4-dihydropteridin-6-yl)methyl)(methyl)amino)benzamido)-4- |
| | (1-(20-(4-(5-(7-chloro-8-(((R)-1-(5-cyano-2-fluorophenyl)ethyl)amino)-3-fluoro-6-methyl- |
| | 1,5-naphthyridin-2-yl)pyrimidin-2-yl)piperazin-1-yl)-20-oxo-3,6,9,12,15,18- |
| | hexaoxaicosyl)-1H-1,2,3-triazol-4-yl)butanoic acid |
| | LCMS m/z 1303.4 [M + H]⁺. |
| I-87 | 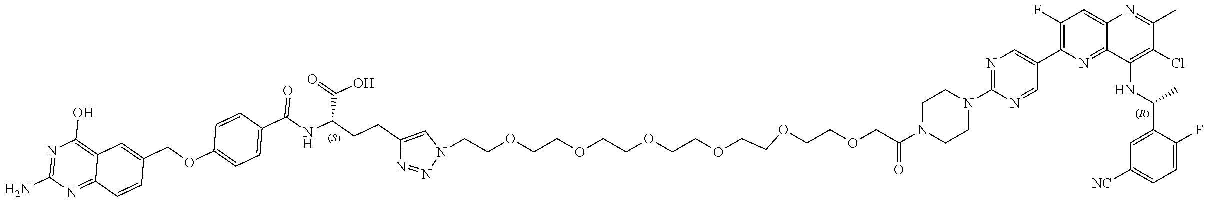 |
| | (S)-2-(4-((2-amino-4-hydroxyquinazolin-6-yl)methoxy)benzamido)-4-(1-(20-(4-(5-(7- |
| | chloro-8-(((R)-1-(5-cyano-2-fluorophenyl)ethyl)amino)-3-fluoro-6-methyl-1,5- |
| | naphthyridin-2-yl)pyrimidin-2-yl)piperazin-1-yl)-20-oxo-3,6,9,12,15,18-hexaoxaicosyl)- |
| | 1H-1,2,3-triazol-4-yl)butanoic acid |
| | LCMS m/z 645.10 [(M/2) + 1]⁺ |
| | ¹H-NMR (400 MHz, DMSO-d₆ with D₂O exchange) δ 8.77 (s, 2H), 7.96 (d, J = 10.8 Hz, 2H), |
| | 7.89 (d, J = 6.8 Hz, 1H), 7.81-7.77 (m, 4H), 7.75-7.70 (m, 1H), 7.35 (d, J = 8.4 Hz, 1H), 7.27 |
| | (t, J = 10.0 Hz, 1H), 7.03 (d, J = 8.4 Hz, 2H), 6.45-6.40 (m, 1H), 5.17 (s, 2H), 4.42-4.39 (m, |
| | 2H), 4.31-4.30 (m, 1H), 4.17 (s, 2H), 3.85-3.75 (m, 4H), 3.74-3.71 (m, 2H), 3.54-3.50 (m, 8H), |
| | 3.45-3.38 (m, 17H), 2.75-2.70 (m, 1H), 2.61 (s, 3H), 2.13-2.04 (m, 2H), 1.64 (d, J = 6.4 Hz, 3H) |
| I-88 | 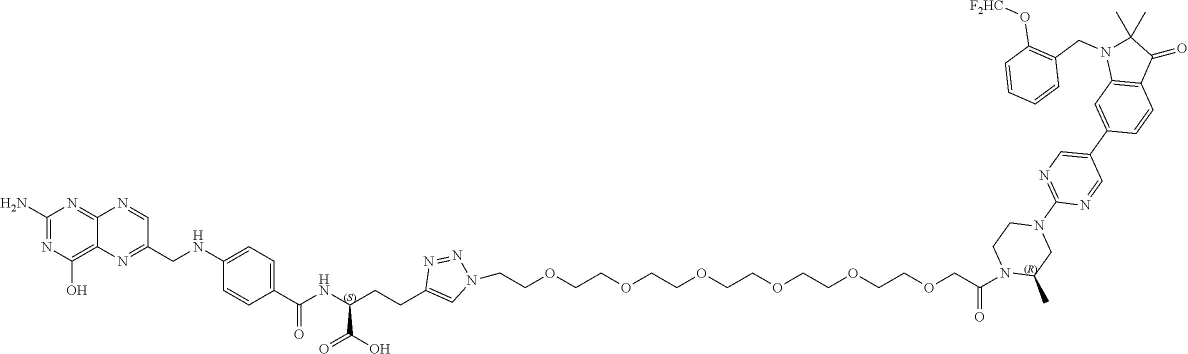 |
| | (S)-2-(4-(((2-amino-4-hydroxypteridin-6-yl)methyl)amino)benzamido)-4-(1-(20-((R)-4-(5- |
| | (1-(2-(difluoromethoxy)benzyl)-2,2-dimethyl-3-oxoindolin-6-yl)pyrimidin-2-yl)-2- |
| | methylpiperazin-1-yl)-20-oxo-3,6,9,12,15,18-hexaoxaicosyl)-1H-1,2,3-triazol-4- |
| | yl)butanoic acid |
| | LCMS m/z 631.90 [(M/2) + 1]⁺ |
| | ¹H NMR (400 MHZ, DMSO-d₆ with D₂O exchange) δ 8.62-8.60 (m, 2H), 7.75 (d, J = 7.2 Hz, |
| | 1H), 7.63-7.54 (m, 2H), 7.35-7.28 (m, 1H), 7.24-7.13 (m, 3H), 6.99 (d, J = 9.6 Hz, 1H), 6.90 (s, |
| | 1H), 6.62 (d, J = 8.8 Hz, 1H), 4.72 (s, 2H), 4.65-4.50 (m, 1H), 4.47 (s, 1H), 4.45-4.35 (m, 3H), |
| | 4.33-4.28 (m, 1H), 4.20-4.05 (m, 2H), 3.73-3.70 (m, 3H), 3.58-3.47 (m, 4H), 3.45-3.37 (m, |
| | 14H), 3.21-3.07 (m, 2H), 2.96-2.84 (m, 1H), 2.15-2.00 (m, 2H), 1.19-1.06 (m. 7H), 1.05-0.96 |
| | (m, 2H) |
| I-89 | 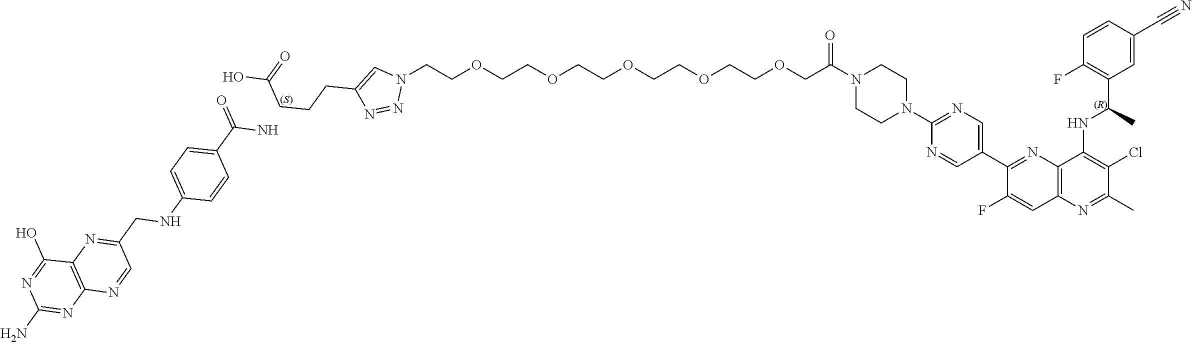 |
| | (S)-2-(4-(((2-amino-4-hydroxypteridin-6-yl)methyl)amino)benzamido)-4-(1-(17-(4-(5-(7- |
| | chloro-8-(((R)-1-(5-cyano-2-fluorophenyl)ethyl)amino)-3-fluoro-6-methyl-1,5- |
| | naphthyridin-2-yl)pyrimidin-2-yl)piperazin-1-yl)-17-oxo-3,6,9,12,15- |
| | pentaoxaheptadecyl)-1H-1,2,3-triazol-4-yl)butanoic acid |
| | LCMS m/z 1245.50 [M + 1] |
| | ¹H NMR (400 MHz, DMSO-d₆ with D₂O exchange) δ 8.78 (s,1H), 8.63 (s, 1H), 8.02 (d, J = 10.8 |
| | Hz, 1H), 7.94 (d, J = 5.6 Hz, 1H), 7.78-7.76 (m, 2H), 7.62 (d, J = 8.8 Hz, 2H), 7.31 (t, J = 10 |
| | Hz, 1H), 6.51 (d, J = 8.8 Hz, 2H), 6.54-6.51 (m, 1H), 4.68 (s, 2H), 4.40 (t, J = 4.8 Hz, 2H), |
| | 4.31-4.27 (m, 1H), 4.18 (s, 2H), 3.89-3.81 (m, 6H), 3.75-3.71 (m, 3H), 3.53-3.52 (m, 8H), 3.45- |
| | 3.41 (m, 12H), 2.67-2.62 (m, 4H), 2.10-2.02 (m, 2H), 1.67 (d, J = 6.8 Hz, 3H) |

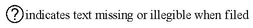

### 6.1.39. Example 39: Compound I-90

### (S)-2-(4-(((2-amino-4-hydroxypteridin-6-yl)methyl)amino)benzamido)-5-(9-(3-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)propyl)-3,9-diazaspiro[5.5]undecan-3-yl)-5-oxopentanoic acid (Compound I-90)

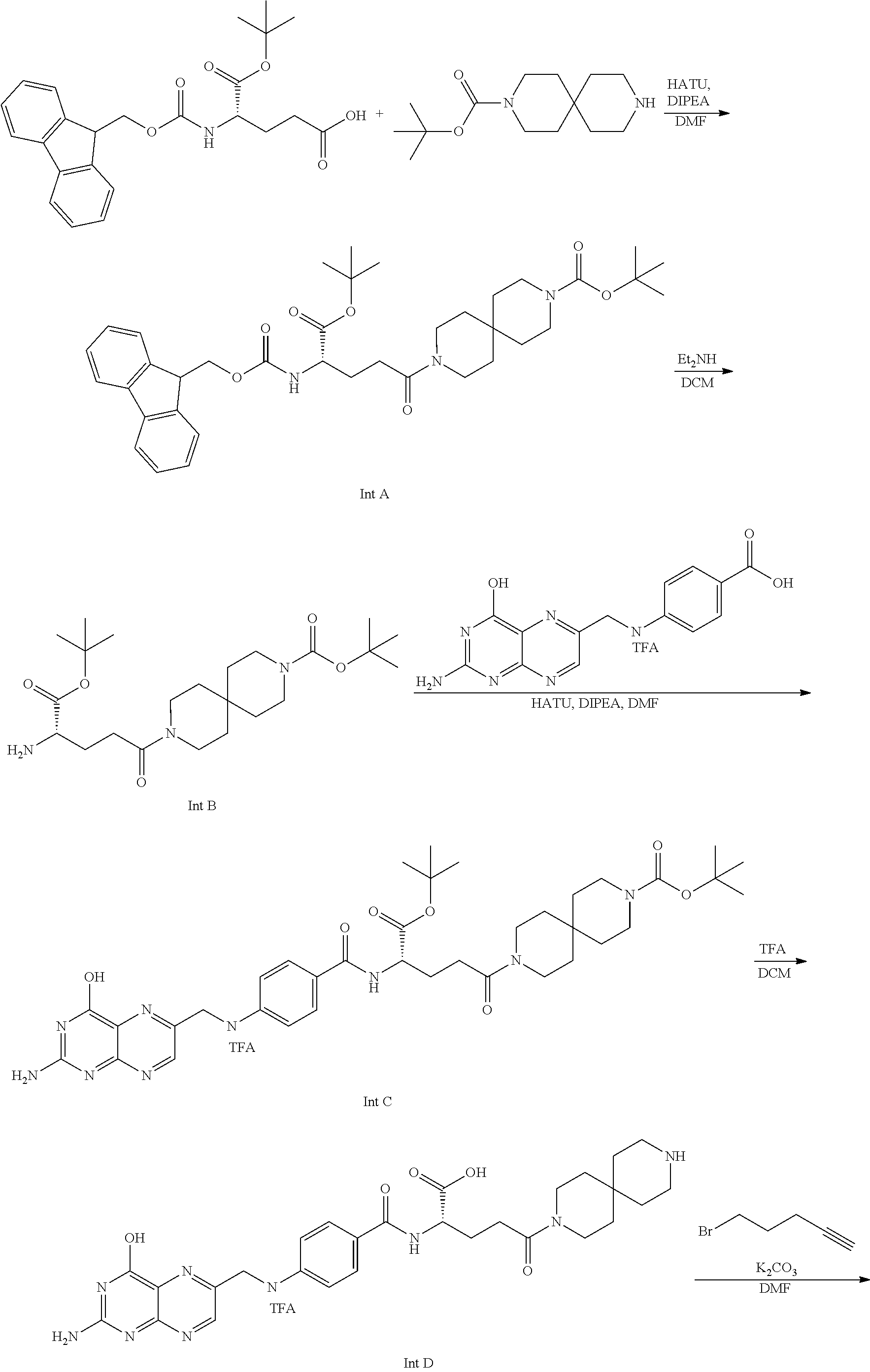

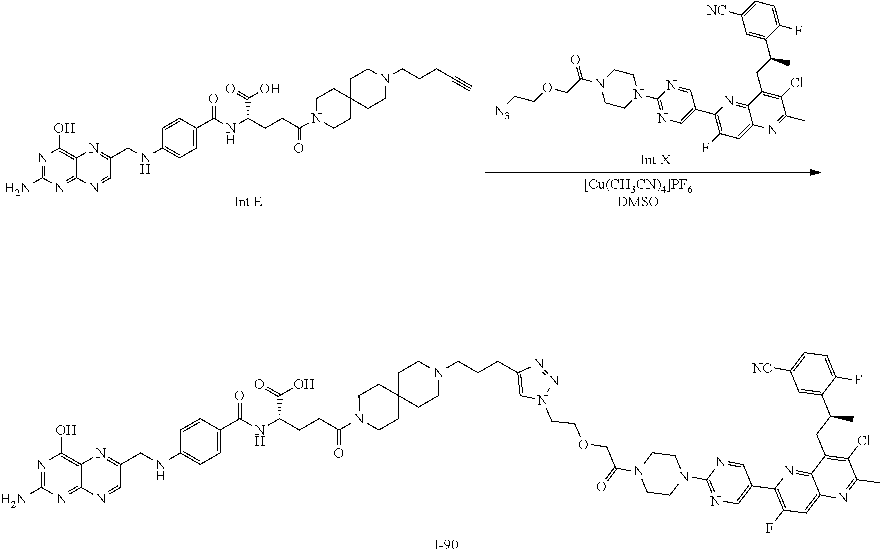

A mixture of (4S)-5-tert-butoxy-4-(9H-fluoren-9-yloxycarbonylamino)-5-oxo-pentanoic acid (1.00 eq, 121 mg, 0.288 mmol) and HATU (1.20 eq, 131 mg, 0.345 mmol) was dissolved in dimethylformamide (2.43 mL) and N,N-diisopropylethylamine (2.00 eq, 100 L, 0.575 mmol) while cooling in an ice bath. After 5 minutes, the ice bath was removed then solid tert-butyl 3,9-diazaspiro[5.5]undecane-3-carboxylate (1.03 eq, 78.5 mg, 0.296 mmol) was added. After 30 minutes, the reaction was diluted with ethyl acetate (25 mL) and washed consecutively with 2% citric acid (40 mL), saturated sodium bicarbonate (40 mL) then saturated sodium chloride (10 mL). The organic phase was then dried over sodium sulfate, filtered, and concentrated under reduced pressure. The residue was purified by flash chromatography (20-100% ethyl acetate in hexanes) to give tert-butyl (S)-9-(4-((((9H-fluoren-9-yl)methoxy)carbonyl)amino)-5-(tert-butoxy)-5-oxopentanoyl)-3,9-diazaspiro[5.5]undecane-3-carboxylate (Int A) Yield: 145 mg, 78%. LCMS m/z 662.4 [M+H]⁺.

A solution of tert-butyl (S)-9-(4-((((9H-fluoren-9-yl)methoxy)carbonyl)amino)-5-(tert-butoxy)-5-oxopentanoyl)-3,9-diazaspiro[5.5]undecane-3-carboxylate (Int A) (1.00 eq, 145 mg, 0.224 mmol) in DCM (0.746 mL) was treated with diethylamine (4.00 eq, 93 L, 0.895 mmol) and the reaction was stirred at room temperature. After 1 h, more diethylamine (10.8 eq, 250 L, 2.42 mmol) was added and the reaction was stirred another hour. Volatile components were removed under reduced pressure to give crude tert-butyl tert-butyl (S)-9-(4-amino-5-(tert-butoxy)-5-oxopentanoyl)-3,9-diazaspiro[5.5]undecane-3-carboxylate (Int B) which was used in the next step without further purification. Yield: 154 mg. LCMS m/z 440.2 [M+H]⁺.

A solution of 4-[(2-amino-4-oxo-1H-pteridin-6-yl)methyl-(2,2,2-trifluoroacetyl)amino]benzoic acid (2.00 eq, 182 mg, 0.446 mmol) and HATU (1.30 eq, 110 mg, 0.290 mmol) in DMA (1.1 mL) was treated with N,N-diisopropylethylamine (1.00 eq, 39 L, 0.223 mmol) followed by a solution of tert-butyl (S)-9-(4-amino-5-(tert-butoxy)-5-oxopentanoyl)-3,9-diazaspiro[5.5]undecane-3-carboxylate (Int B) (1.00 eq, 98.0 mg, 0.223 mmol) in DCM (1.1 mL) and the reaction was stirred at room temp. After 1 h, the reaction was diluted with ethyl acetate (20 mL) and 10% citric acid (20 mL) and the resulting organic phase was washed consecutively with saturated sodium bicarbonate and ½ saturated NaCl then filtered and concentrated under reduced pressure. The residue was purified by reversed-phase HPLC (10-100% ACN in water w/0.1% formic acid) to give tert-butyl (S)-9-(4-(4-(N-((2-amino-4-hydroxypteridin-6-yl)methyl)-2,2,2-trifluoroacetamido)benzamido)-5-(tert-butoxy)-5-oxopentanoyl)-3,9-diazaspiro[5.5]undecane-3-carboxylate (Int C). Yield: 142 mg, 77%. LCMS m/z 830.4 [M+H]⁺.

A solution of tert-butyl (S)-9-(4-(4-(N-((2-amino-4-hydroxypteridin-6-yl)methyl)-2,2,2-trifluoroacetamido)benzamido)-5-(tert-butoxy)-5-oxopentanoyl)-3,9-diazaspiro[5.5]undecane-3-carboxylate (Int C) (1.00 eq, 142 mg, 0.171 mmol) in DCM (856 L) was treated with trifluoroacetic acid (50.0 eq, 655 L, 8.56 mmol) for 1 hour. Volatile components were removed under reduced pressure and the residue was concentrated from isopropanol (10 mL) then again from DCM (20 mL) then dried under high vacuum to give (S)-2-(4-(N-((2-amino-4-hydroxypteridin-6-yl)methyl)-2,2,2-trifluoroacetamido)benzamido)-5-oxo-5-(3,9-diazaspiro[5.5]undecan-3-yl)pentanoic acid (Int D). Yield: 152 mg. LCMS m/z 674.2 [M+H]⁺.

A solution (S)-2-(4-(N-((2-amino-4-hydroxypteridin-6-yl)methyl)-2,2,2-trifluoroacetamido)benzamido)-5-oxo-5-(3,9-diazaspiro[5.5]undecan-3-yl)pentanoic acid (Int D) (1.00 eq, 100 mg, 0.148 mmol) in DMSO (0.145 mL) at room temperature was treated with potassium carbonate (3.00 eq, 61.6 mg, 0.445 mmol) before adding 5-bromopent-1-yne (1.05 eq, 18 L, 0.156 mmol). After 48 h and several additional charges of alkyl bromide later, the reaction solution was filtered and purified by reversed-phase HPLC (10-50% ACN in water w/0.1% TFA) to give (S)-2-(4-(((2-amino-4-hydroxypteridin-6-yl)methyl)amino)benzamido)-5-oxo-5-(9-(pent-4-yn-1-yl)-3,9-diazaspiro[5.5]undecan-3-yl)pentanoic acid (Int E). Yield: 30 mg, 30%. LCMS m/z 644.3 [M+H]⁺.

A solution of (S)-2-(4-(((2-amino-4-hydroxypteridin-6-yl)methyl)amino)benzamido)-5-oxo-5-(9-(pent-4-yn-1-yl)-3,9-diazaspiro[5.5]undecan-3-yl)pentanoic acid (Int E) (1.05 eq, 30.4 mg, 0.0489 mmol) and tetrakis(acetonitrile)copper(I) hexafluorophosphate (0.50 eq, 8.7 mg, 0.0233 mmol) in DMSO (233 L) was treated with a solution of (2S)-2-[4-[(2-amino-4-hydroxy-pteridin-6-yl)methylamino]benzoyl]oxy-5-oxo-5-(9-pent-4-ynyl-3,9-diazaspiro[5.5]undecan-3-yl)pentanoic acid (Int X) (1.00 eq, 30.0 mg, 0.0465 mmol) in DMSO (233 L) and the reaction was stirred at room temperature for several hours. The reaction solution was acidified with formic acid then purified by reversed-phase HPLC (5-50% acetonitrile in water w/0.1% formic acid) to give the title compound (S)-2-(4-(((2-amino-4-hydroxypteridin-6-yl)methyl)amino)benzamido)-5-(9-(3-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)propyl)-3,9-diazaspiro[5.5]undecan-3-yl)-5-oxopentanoic acid (Compound I-90) as a white solid. Yield: 14 mg, 24%. LCMS m/z 646.6 [M+2H/2]⁺.

### 6.1.40. Example 40: Compound I-91

### (S)-18-((1r,4S)-4-(((2-amino-4-hydroxypteridin-6-yl)methyl)amino)cyclohexane-1-carboxamido)-1-(1-(2-(2-(4-(5-(7-chloro-8-(((S)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)-15-oxo-2,5,8,11-tetraoxa-14-azanonadecan-19-oic acid (Compound I-91)

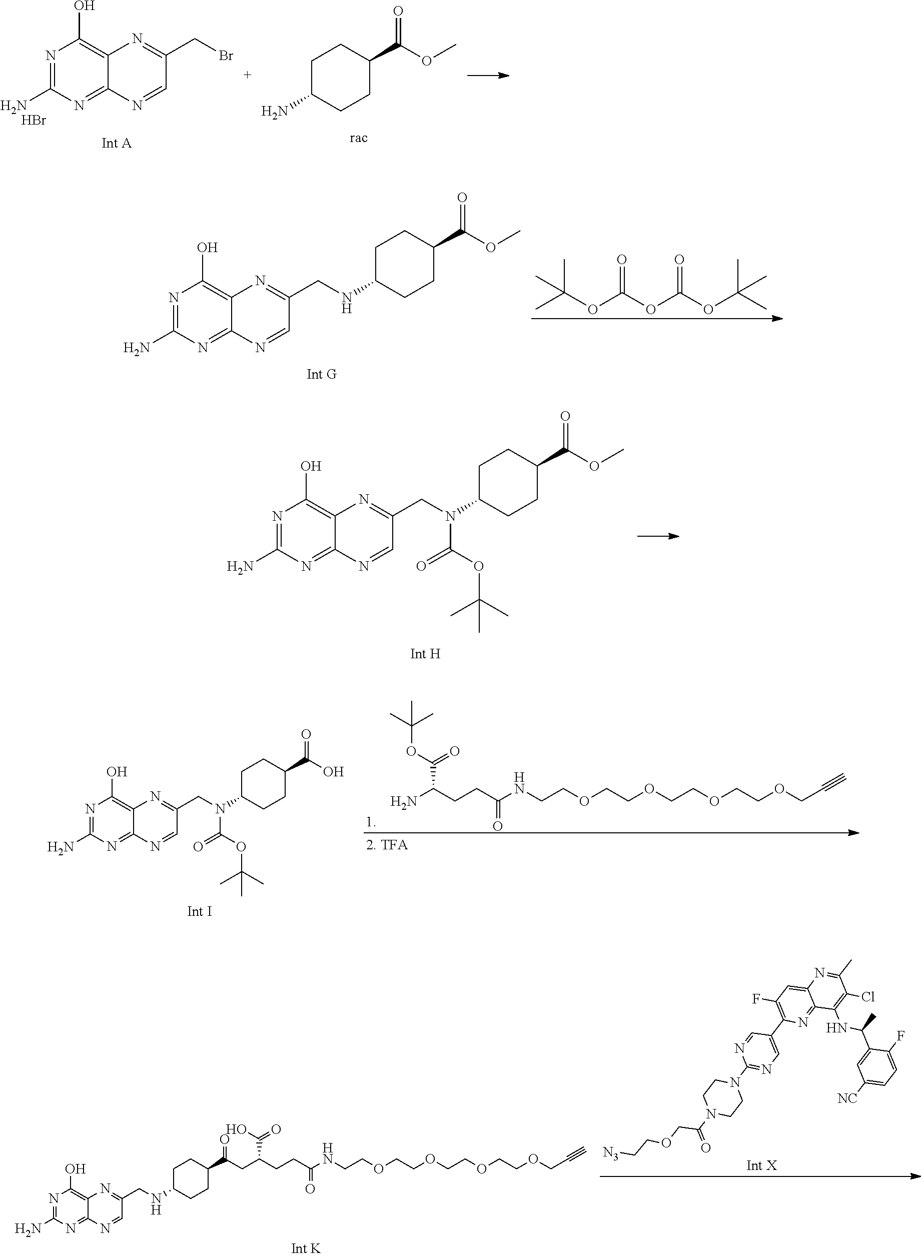

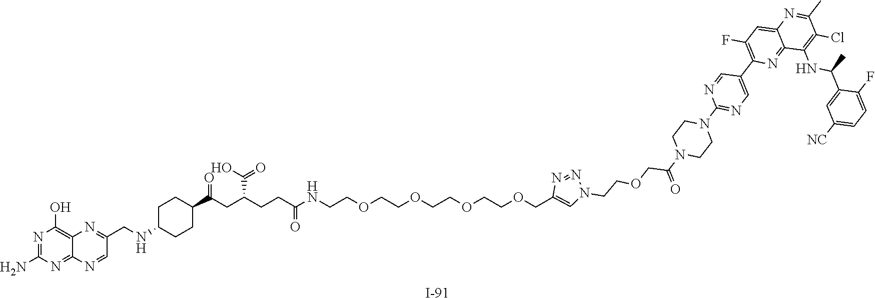

A solution of methyl 4-aminocyclohexanecarboxylate (3.0 eq, 196 mg, 1.25 mmol) in DMF (4.15 mL) was cooled in an ice bath then treated with 2-amino-6-(bromomethyl)pteridin-4-ol hydrobromide (Int A) (1.0 eq, 140 mg, 0.415 mmol) before removing the ice bath. After 25 minutes, the reaction was dripped into diethyl ether (40 mL) and the precipitate collected by filtration. The precipitate was dissolved in dimethyl sulfoxide then purified by reversed-phase HPLC (5-20% acetonitrile in water w/0.1% TFA) to give methyl (1r,4r)-4-(((2-amino-4-hydroxypteridin-6-yl)methyl)amino)cyclohexane-1-carboxylate (Int G) as the TFA salt. Yield: 140 mg, 76%. LCMS m/z 333.2 [M+H]⁺.

A mixture of Int G (1.0 eq, 135 mg, 0.302 mmol), cesium carbonate (3.0 eq, 296 mg, 0.907 mmol) and di-tert-butyl dicarbonate (1.7 eq, 112 mg, 0.514 mmol) in DMF (2.6 mL) water (1 mL) and THF (1 mL) was stirred at room temperature for 1 hour before adding a pinch of DMAP. The reaction was stirred for 18 hours then concentrated under high vacuum. The residue was dissolved in DMSO, filtered then purified by reversed-phase HPLC to give methyl (1r,4r)-4-(((2-amino-4-hydroxypteridin-6-yl)methyl)(tert-butoxycarbonyl)amino)cyclohexane-1-carboxylate (Int H). Yield: 41 mg, 31%. LCMS m/z 433.3 [M+H]⁺.

A solution of Int H (1.00 eq, 41.0 mg, 0.0948 mmol) in methanol (948 L) was treated with 1M aqueous sodium hydroxide (3.0 eq, 284 L, 0.284 mmol). After 3 hours, the solution was concentrated under reduced pressure to give (S)-20-((1r,4S)-4-(((2-amino-4-hydroxypteridin-6-yl)methyl)amino)cyclohexane-1-carboxamido)-17-oxo-4,7,10,13-tetraoxa-16-azahenicos-1-yn-21-oic acid (Int I) as a white solid. LCMS: m/z 419.3 [M+H]⁺. The residue was treated with HATU (1.30 eq, 46.1 mg, 0.121 mmol) and N,N-diisopropylethylamine (2.00 eq, 32 L, 0.186 mmol) and tert-butyl(2S)-2-amino-5-oxo-5-[2-[2-[2-(2-prop-2-ynoxyethoxy)ethoxy]ethoxy]ethylamino]pentanoate (1.20 eq, 46.6 mg, 0.112 mmol) in DMF (364 L). After 22 hours, the solution was filtered then purified by reversed-phase HPLC (10-100% acetonitrile in water w/0.1% FA) to give tert-butyl (S)-20-((1r,4S)-4-(((2-amino-4-hydroxypteridin-6-yl)methyl)(tert-butoxycarbonyl)amino)cyclohexane-1-carboxamido)-17-oxo-4,7,10,13-tetraoxa-16-azahenicos-1-yn-21-oate (Int J). Yield: 29 mg, 38%. LCMS m/z 817.4 [M+H]⁺.

A mixture of Int J (1.0 eq, 22.0 mg, 0.0269 mmol) and trifluoroacetic acid (80.0 eq, 164 L, 2.15 mmol) in DCM (164 L) was stirred at room temperature for 1 h before being concentrated under high vacuum. The crude material was dissolved in and concentrated from water (5 mL) then again from acetonitrile (5 mL) to give crude tert-butyl (S)-20-((1r,4S)-4-(((2-amino-4-hydroxypteridin-6-yl)methyl)(tert-butoxycarbonyl)amino)cyclohexane-1-carboxamido)-17-oxo-4,7,10,13-tetraoxa-16-azahenicos-1-yn-21-oate (Int K) which was used in next step without further purification.

A solution of Int K (1.05 eq, 12.7 mg, 0.0203 mmol) and tetrakis(acetonitrile)copper(I) hexafluorophosphate (0.500 eq, 3.6 mg, 0.00967 mmol) in DMSO (97 L) was treated with a solution of (S)-3-(1-((6-(2-(4-(2-(2-azidoethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (Int X) (1.0 eq, 15.0 mg, 0.0193 mmol) in DMSO (97 μL) and the reaction was stirred at room temp for several hours. The reaction was acidified, filtered then purified by reversed-phase HPLC (5-50% acetonitrile in water w/0.1% FA) to give (2S)-2-[4-[(2-amino-4-hydroxy-pteridin-6-yl)methylamino]cyclohexanecarbonyl]oxy-5-[2-[2-[2-[2-[[1-[2-[2-[4-[5-[7-chloro-3-fluoro-8-[[(1R)-1-(2-fluorophenyl)ethyl]amino]-6-methyl-1,5-naphthyridin-2-yl]pyrimidin-2-yl]piperazin-1-yl]-2-oxo-ethoxy]ethyl]triazol-4-yl]methoxy]ethoxy]ethoxy]ethoxy]ethylamino]-5-oxo-pentanoic acid (Compound I-91). Yield: 14 mg; 56% from Int D. LC-MS; m/z 654.8 [(M+2H/2)]⁺.

Compound I-45 was prepared by adapting the general method described above for compound I-91. The structure, name and data for compound I-45 is provided below:

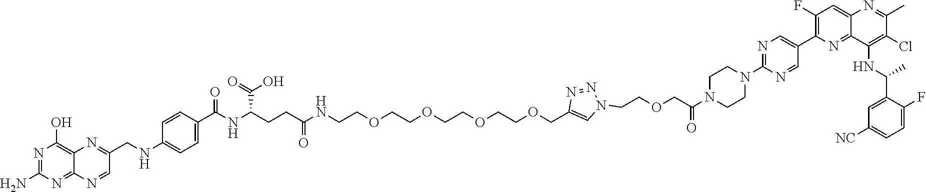 [SMILES: Cc1nc2cc(F)c(-c3cnc(N4CCN(C(=O)COCCn5cc(COCCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl]

### (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)-15-oxo-2,5,8,11-tetraoxa-14-azanonadecan-19-oic acid (Compound I-45)

LCMS m/z 651.76 [(M/2)+1]⁺.

¹H NMR (400 MHz, DMSO-d₆ with D₂O exchange) δ 8.75 (d, J=6.4 Hz, 2H), 8.59 (s, 1H), 8.06 (s, 1H), 7.97 (d, J=11.2 Hz, 1H), 7.87 (d, J=6.8 Hz, 1H), 7.72-7.69 (m, 1H), 7.56 (d, J=8.8 Hz, 2H), 7.27 (t, J=9.6 Hz, 1H), 6.60-6.54 (m, 2H), 6.46-6.42 (m, 1H), 4.53 (t, J=4.4 Hz, 2H), 4.47-4.44 (m, 4H), 4.24-4.21 (m, 1H) 4.19 (s, 2H), 3.85 (t, J=4.4 Hz, 2H), 3.79 (bs, 2H), 3.72 (bs, 2H), 3.50-3.45 (m, 4H), 3.44-3.39 (m, 11H), 3.32-3.29 (m, 4H), 3.12-3.10 (m, 2H), 2.61 (s, 3H), 2.17 (t, J=6.8 Hz, 2H), 1.99-1.88 (m, 2H), 1.65 (d, J=6.4 Hz, 3H).

### 6.1.41. Example 41: Compound I-96

### (R)-4-(((2-amino-4-hydroxypteridin-6-yl)methyl)amino)-N-(1-(1-(2-(2-(4-(5-(7-chloro-8-((1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)-15-oxo-2,5,8,11-tetraoxa-14-azaoctadecan-18-yl)benzamide (Compound I-96)

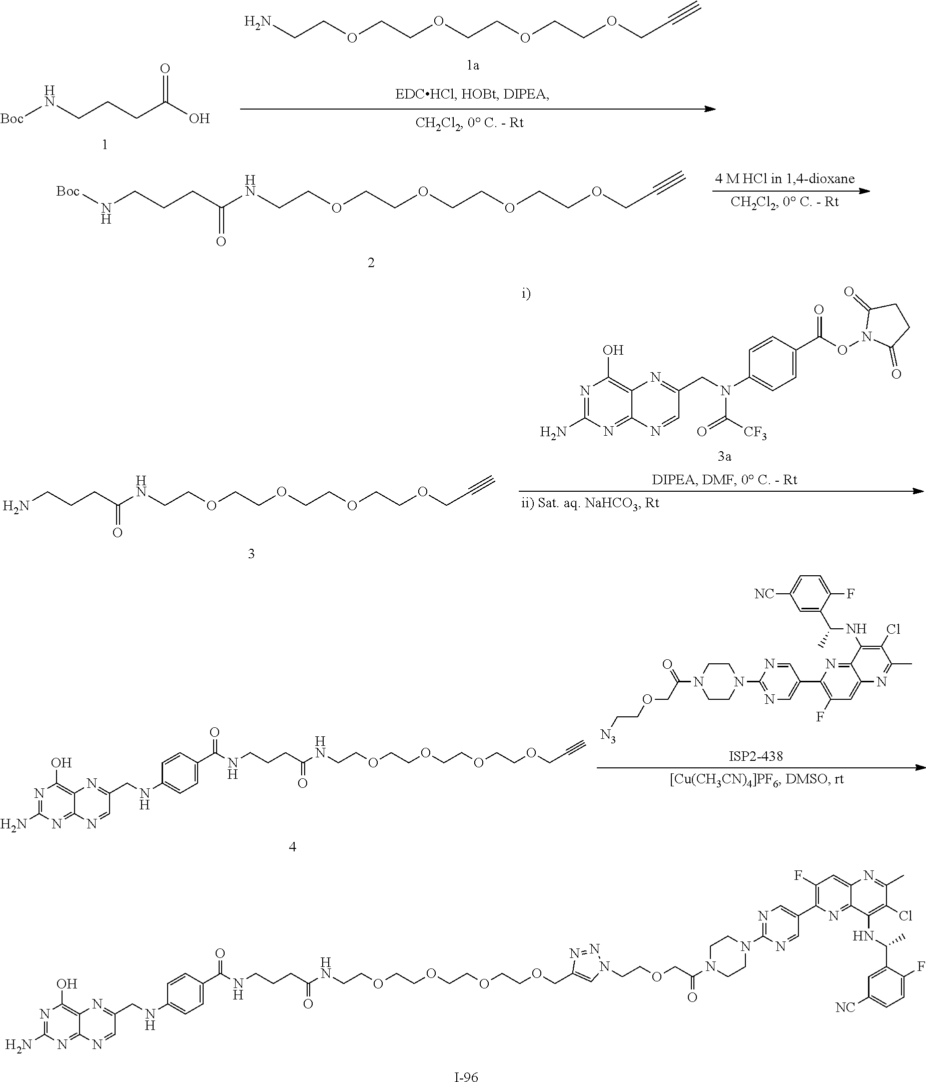

To a stirred solution of 4-((tert-butoxycarbonyl)amino)butanoic acid (1, 0.5 g, 2.46 mmol, 1.0 eq) in anhydrous dichloromethane (25 mL), was added 1H-1,2,3-benzotriazol-1-ol (0.499 g, 3.69 mmol, 1.5 eq.) at 0° C. under nitrogen atmosphere, and stirred for 15 minutes. Then N-(3-Dimethylaminopropyl)-N′-ethylcarbodiimide hydrochloride (EDC·HCl)(0.707 g, 3.69 mmol, 1.5 eq.) was added. After 15 minutes, N,N-Diisopropylethylamine (1.74 mL, 9.84 mmol, 4 eq.), and 3,6,9,12-tetraoxapentadec-14-yn-1-amine (0.626 g, 2.71 mmol, 1.1 eq) were added to the reaction mixture and stirred the reaction mixture for 12 h at room temperature. The volatilee were evaporated to give crude residue which was purified by silica gel flash column chromatography using 10% methanol in dichloromethane to afford tert-butyl (17-oxo-4,7,10,13-tetraoxa-16-azaicos-1-yn-20-yl)carbamate (2) as viscous liquid. Yield: 0.75 g 73.1%; LCMS-MS m/z 416.2 [M+1]⁺.

To a stirred solution of tert-butyl (17-oxo-4,7,10,13-tetraoxa-16-azaicos-1-yn-20-yl)carbamate (2, 0.9 g, 2.16 mmol, 1.0 eq.) in dichloromethane (10 mL) was added hydrogen chloride solution (4M in 1,4-dioxane, 3 mL) at room temperature and the reaction was stirred at same temperature for 3 h. The reaction was monitored by TLC and after completion, the volatiles were evaporated in high vaccuo, and azeotrope the mixture 3-4 times with anhydrous dichloromethane to obtained the crude 4-amino-N-(3,6,9,12-tetraoxapentadec-14-yn-1-yl)butanamide (3) as viscous liquid. Yield: 0.76 g, Crude; LCMS-MS 317.12 [M+1]⁺.

To a suspension of 2,5-dioxopyrrolidin-1-yl 4-(N-((2-amino-4-hydroxypteridin-6-yl)methyl)-2,2,2-trifluoroacetamido)benzoate (3a, 0.40 g, 0.791 mmol, 1.0 eq.), and 4-amino-N-(3,6,9,12-tetraoxapentadec-14-yn-1-yl)butanamide (3, 0.335 g, 0.950 mmol, 1.2 eq) in N,N-dimethylformamide (5 mL), N,N-diisopropylethylamine (0.689 mL, 3.96 mmol, 5.0 eq) was added and reaction mixture was stirred at room temperature for 16 h. The progress of the reaction was monitored by LCMS. After completion, saturated aq. sodium bicarbonate (2 mL) was added and the reaction mixture was stirred at room temperature for 2 h. After completion (monitored by LCMS), the reaction mixture was concentrated under reduced pressure and purified by prep-HPLC (with 8-35% acetonitrile in water with 5 mm ammonium acetate). The desired fractions were collected and lyophilized to dryness to afford 4-(((2-amino-4-hydroxypteridin-6-yl)methyl)amino)-N-(17-oxo-4,7,10,13-tetraoxa-16-azaicos-1-yn-20-yl)benzamide (4) as yellow solid. Yield: 0.2 g, 41.3%; LCMS m/z 611.24 [M+1]⁺

To a solution of 4-(((2-amino-4-hydroxypteridin-6-yl)methyl)amino)-N-(17-oxo-4,7,10,13-tetraoxa-16-azaicos-1-yn-20-yl)benzamide (4, 0.06 g, 0.98 mmol, 1.0 eq.) and (R)-3-(1-((6-(2-(4-(2-(2-azidoethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (ISP2-438, 0.0637 g, 0.98 mmol, 1.0 eq.) in dimethylsulfoxide (1 mL), tetrakis(acetonitrile)copper(I) hexafluorophosphate (0.0549 g, 0.14 mmol, 1.5 eq.) was added and reaction mixture was stirred at room temperature for 1 h. Thereafter, the reaction mixture was purified by prep. HPLC (using 20-40% acetonitrile in water with 0.10% TFA). Fractions containing the desired product were combined and lyophilized to dryness to afford (R)-4-(((2-amino-4-hydroxypteridin-6-yl)methyl)amino)-N-(1-(1-(2-(2-(4-(5-(7-chloro-8-((1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)-15-oxo-2,5,8,11-tetraoxa-14-azaoctadecan-18-yl)benzamide (Compound I-96) as yellow solid. Yield: 0.036 g, 29.1%; LCMS m/z 1258.55 [(M+1]⁺, ¹H NMR (400 MHz, DMSO-d₆) δ11.4 (brs, 1H), 8.94 (s, 2H), 8.62 (s, 1H), 8.14 (s, 1H), 8.08 (d, J 12.0 Hz, 1H), 8.04 (dd, J 6.8, 2.0 Hz, 1H), 8.00 (t, J 6.0 Hz, 1H), 7.85 (t, J 5.6 Hz, 1H), 7.80-7.78 (m, 1H), 7.59 (d, J 8.8 Hz, 1H), 7.35 (t, J=8.8 Hz, 1H), 6.95 (d, J=9.2 Hz, 1H), 6.85 (t, J=4.8 Hz, 1H), 6.60 (d, J 8.8 Hz, 2H), 6.33 (t, J=7.6 Hz, 1H), 4.50-4.49 (m, 2H), 4.51 (s, 2H), 4.48 (d, J=5.6 Hz, 2H), 4.25 (s, 2H), 3.91 (t, J=4.8 Hz, 2H), 3.89-3.82 (m, 4H), 3.56-3.53 (m, 6H), 3.51-3.47 (m, 8H), 3.41-3.34 (m, 6H), 3.17-3.14 (m, 4H), 2.61 (s, 3H), 2.08 (t, J=7.6 Hz, 2H), 1.69-1.66 (m, 5H).

### 6.1.42. Example 42: Compound I-98

### (S)-18-(4-((2-(2-amino-4-oxo-4,7-dihydro-3H-pyrrolo[2,3-d]pyrimidin-6-yl)ethyl)thio)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)-15-oxo-2,5,8,11-tetraoxa-14-azanonadecan-19-oic acid (Compound I-98)

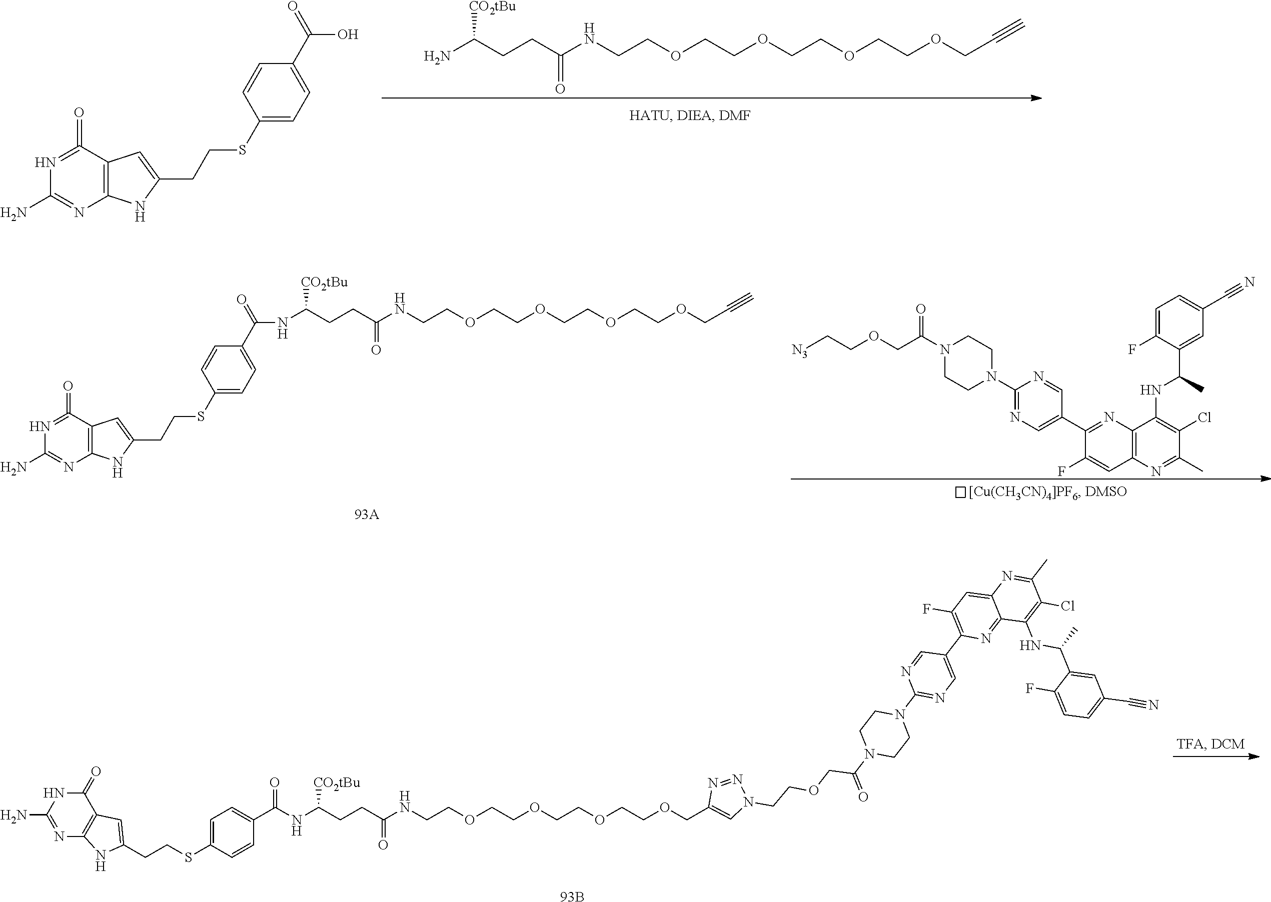

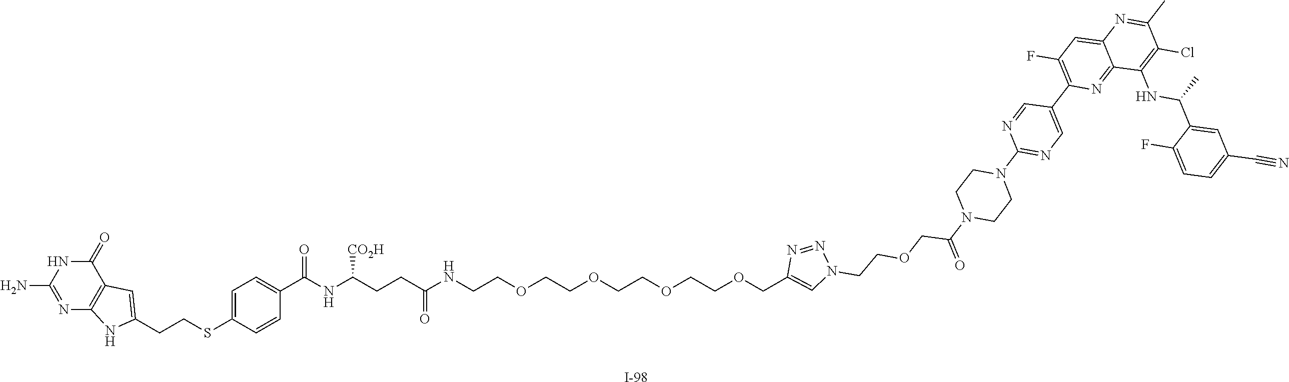

To a mixture of 4-[2-(2-amino-4-oxo-3,7-dihydropyrrolo[2,3-d]pyrimidin-6-yl)ethylsulfanyl]benzoic acid (1.00 eq, 36.8 mg, 0.11 mmol) and tert-butyl (2S)-2-amino-5-oxo-5-[2-[2-[2-(2-prop-2-ynoxyethoxy)ethoxy]ethoxy]ethylamino]pentanoate (1.2 eq, 55.7 mg, 0.13 mmol) in DMF (1 mL) were added DIEA (3.0 eq, 58.2 mL, 0.33 mmol) and HATU (1.2 eq, 50.8 mg, 0.13 mmol). The mixture was stirred at room temperature for 1 h and purified by prep. HPLC (20-55% MeCN/water with 0.1% formic acid) to give 93A as a pink solid (45.2 mg, yield: 55.7%). LCMS m/z 729.4 [M+H]⁺.

To a mixture of tert-butyl (2S)-2-[[4-[2-(2-amino-4-oxo-3,7-dihydropyrrolo[2,3-d]pyrimidin-6-yl)ethylsulfanyl]benzoyl]amino]-5-oxo-5-[2-[2-[2-(2-prop-2-ynoxyethoxy)ethoxy]ethoxy]ethylamino]pentanoate (93A, 1.00 eq, 23.2 mg, 0.032 mmol) and 3-[(1R)-1-[[6-[2-[4-[2-(2-azidoethoxy)acetyl]piperazin-1-yl]pyrimidin-5-yl]-3-chloro-7-fluoro-2-methyl-1,5-naphthyridin-4-yl]amino]ethyl]-4-fluoro-benzonitrile (1.00 eq, 20.6 mg, 0.032 mmol) in DMSO (0.4 mL) was added Tetrakis(acetonitrile)copper(I) hexafluorophosphate (0.5 eq, 5.9 mg, 0.016 mmol). The mixture was stirred at room temperature for 1 h, purified by prep. HPLC (20-55% MeCN/water with 0.1% formic acid) to give 93B as a yellow solid (33.2 mg, yield: 75.8%). LCMS m/z 1376.8 [M+H]⁺.

To a mixture of tert-butyl (2S)-2-[[4-[2-(2-amino-4-oxo-3,7-dihydropyrrolo[2,3-d]pyrimidin-6-yl)ethylsulfanyl]benzoyl]amino]-5-[2-[2-[2-[2-[[1-[2-[2-[4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]piperazin-1-yl]-2-oxo-ethoxy]ethyl]triazol-4-yl]methoxy]ethoxy]ethoxy]ethoxy]ethylamino]-5-oxo-pentanoate (93B, 1.00 eq, 33.2 mg, 0.024 mmol) in DCM (0.2 mL) was added TFA (0.4 mL). The mixture was stirred at room temperature for 1.5 h, concentrated, and purified by prep. HPLC (20-55% MeCN/water with 0.1% TFA) to give Compound I-98 TFA salt as a yellow solid (28.1 mg, yield: 81%). LCMS m/z 660.9 [M/2+H]⁺.

### 6.1.43. Example 43: Compound I-93

### (S)-19-(4-(((2-amino-4-hydroxypteridin-6-yl)methyl)amino)benzamido)-3-((1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)methyl)-1-hydroxy-16-oxo-6,9,12-trioxa-3,15-diazaicosan-20-oic acid (Compound I-93)

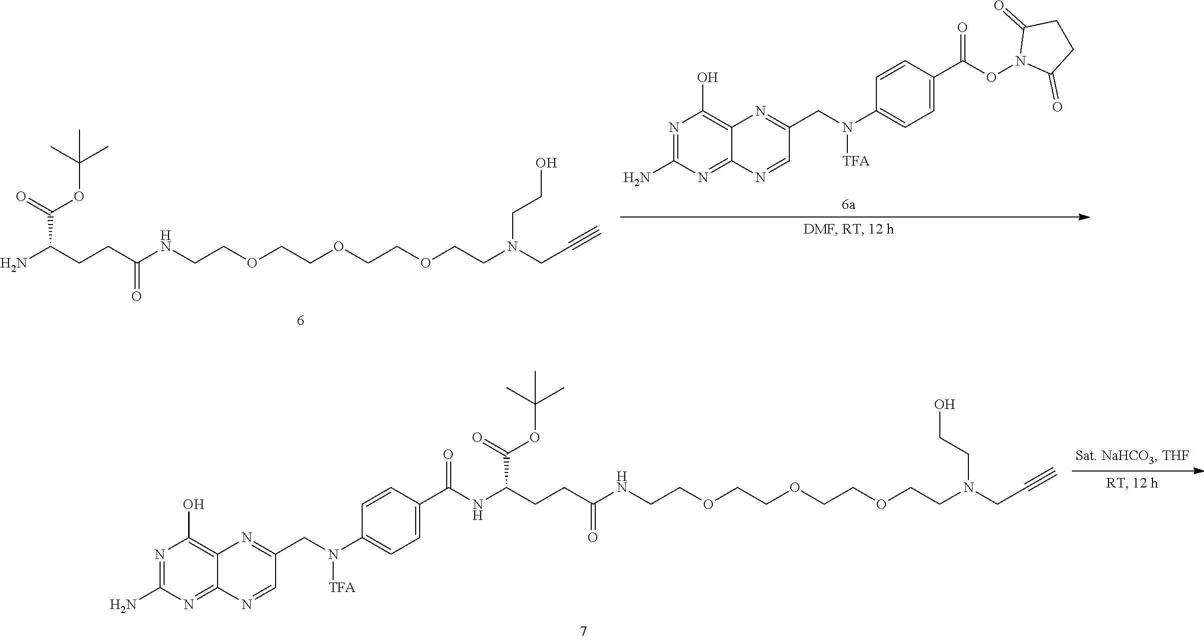

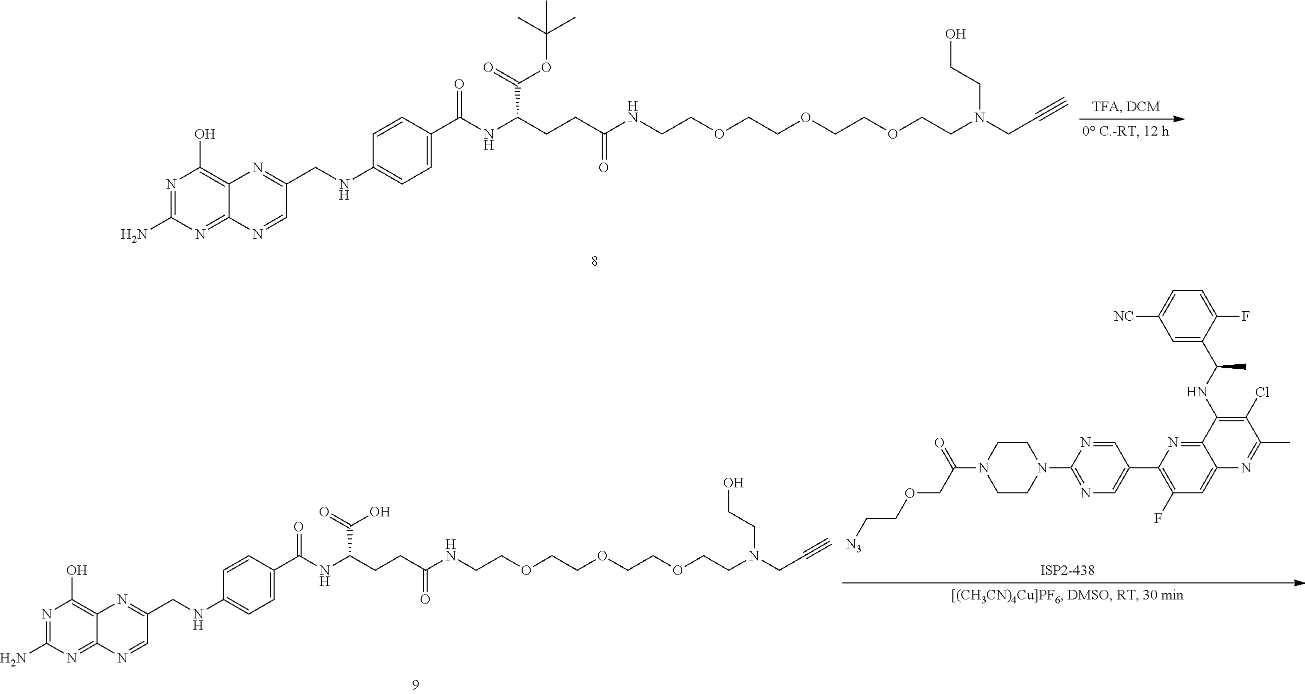

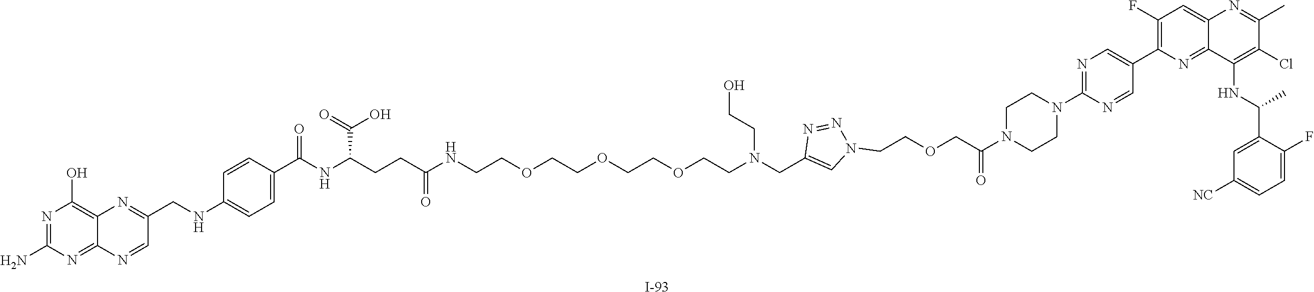

### Synthesis of tert-butyl (S)-20-amino-4-(2-hydroxyethyl)-17-oxo-7,10,13-trioxa-4,16-diazahenicos-1-yn-21-oate (6)

To a stirred solution of tert-butyl (2-(2-(2-(2-hydroxyethoxy)ethoxy)ethoxy)ethyl)carbamate (5 g, 17 mmol) in dichloromethane (50 mL), triethylamine (4.79 mL, 2 eq., 34.1 mmol) followed by methanesulfonyl chloride (1.98 mL, 1.5 eq., 25.6 mmol) was added at 0° C. and reaction mixture was stirred at room temperature for 1.5 h. After the completion of reaction, reaction mixture was concentrated under reduced pressure to get crude 2,2-dimethyl-4-oxo-3,8,11,14-tetraoxa-5-azahexadecan-16-yl methanesulfonate as yellow syrup. The crude was proceeded as such for the next step. Yield=6.3 g (Crude); LCMS-MS m/z 372.2 [M+1].

To a solution of 2,2-dimethyl-4-oxo-3,8,11,14-tetraoxa-5-azahexadecan-16-yl methanesulfonate (4.0 g, 10.8 mmol) in ethanol (80 mL, 1.37 mol) was added N,N-diisopropylethylamine (1.18 mL, 5 eq., 6.73 mmol) and 2-(prop-2-yn-1-ylamino)ethan-1-ol (0.961 g, 0.9 eq., 9.69 mmol). Reaction mixture was stirred at 80° C. for 12 h. After completion, reaction mixture was concentrated under vacuum and residue was purified by silica gel (100-200 mesh) column chromatography using 10-100% ethylacetate/hexane to afford tert-butyl (12-(2-hydroxyethyl)-3,6,9-trioxa-12-azapentadec-14-yn-1-yl)carbamate as yellow oil. Yield: 1.62 g, 40.17%. LCMS m/z 375.2 [M+1].

To a solution of 2-aminoethan-1-ol (3.05 mL, 2 eq., 50.4 mmol) in dichloromethane (150 ml) was added 3-bromoprop-1-yne (1.91 mL, 25.2 mmol) at 0° C. dropwise over 30 min. Then reaction mixture was stirred at RT for 6 h, After completion, mixture was concentrated and the residue was purified by silica gel (100-200 mesh) column chromatography (0 to 2% of methanol in dichloromethane as eluent) to give 2-[(prop-2-yn-1-yl)amino]ethan-1-ol as yellow oil. Yield: 1.00 g, 40.0%. ¹H NMR (400 MHz, CDCl₃) 3.67 (t, J=5.2 Hz, 2H), 3.45 (d, J=2.4 Hz, 2H), 2.85 (t, J=5.2 Hz, 2H), 2.64 (s, 2H), 2.23 (t, J=2.4 Hz, 1H).

To a solution of tert-butyl tert-butyl (12-(2-hydroxyethyl)-3,6,9-trioxa-12-azapentadec-14-yn-1-yl)carbamate (1.6 g, 4.27 mmol) in 1,4-dioxane (10 mL, 156.0 mmol) was added 4M HCl in 1,4-dioxane (10 mL) at 0° C. Reaction mixture was stirred for 2 h at room temperature, and after completion, reaction mixture was concentrated under vacuum to afford crude 14-amino-3-(prop-2-yn-1-yl)-6,9,12-trioxa-3-azatetradecan-1-ol hydrochloride as brown oil. Yield: 1.4 g, Crude; LCMS m/z 275.2 [M+1].

A solution of 1-(tert-butyl) 5-(2,5-dioxopyrrolidin-1-yl) (((9H-fluoren-9-yl)methoxy)carbonyl)-L-glutamate (2.37 g, 4.53 mmol) and 14-amino-3-(prop-2-yn-1-yl)-6,9,12-trioxa-3-azatetradecan-1-ol hydrochloride (1.4 g, 4.53 mmol) in tetrahydrofuran (4 mL) was cooled at 0° C., N,N-diisopropylethylamine (1.67 mL, 2 eq., 9.07 mmol) was added and reaction mixture was stirred at room temperature for 3 h. After completion, reaction mixture was concentrated and the residue was purified by silica gel (100-200 mesh) column chromatography using 1-10% methanol/dichloromethane to afford tert-butyl (S)-20-((((9H-fluoren-9-yl)methoxy)carbonyl)amino)-4-(2-hydroxyethyl)-17-oxo-7,10,13-trioxa-4,16-diazahenicos-1-yn-21-oate as colorless oil. Yield: 1.7 g, 39.6%; LCMS m/z 682.3 [M+1].

To a solution of tert-butyl (S)-20-((((9H-fluoren-9-yl)methoxy)carbonyl)amino)-4-(2-hydroxyethyl)-17-oxo-7,10,13-trioxa-4,16-diazahenicos-1-yn-21-oate (0.850 g, 1.25 mmol) in methanol (10 mL), diethyl amine (2.61 mL, 20 eq., 24.9 mmol) was added and reaction mixture was stirred at room temperature for 4 h. After completion, reaction mixture was concentrated under reduced pressure till dry to afford tert-butyl (S)-20-amino-4-(2-hydroxyethyl)-17-oxo-7,10,13-trioxa-4,16-diazahenicos-1-yn-21-oate (6) as a colorless viscous liquid. Yield: 0.650 g, Crude. LCMS-MS m/z 460.7 [M+1].

### Synthesis of Compound I-93

To a of solution tert-butyl (S)-20-amino-4-(2-hydroxyethyl)-17-oxo-7,10,13-trioxa-4,16-diazahenicos-1-yn-21-oate (6, 0.641 g, 1.5 eq., 1.4 mmol) in N,N-dimethylformamide (10 mL) was added 2,5-dioxopyrrolidin-1-yl 4-(N-((2-amino-4-hydroxypteridin-6-yl)methyl)-2,2,2-trifluoroacetamido)benzoate (6a, 0.470 g, 0.930 mmol) and reaction mixture was stirred at room temperature for overnight. After completion, reaction mixture was concentrated and residue was purified on silica gel (100-200 mesh) column chromatography using 1-10% methanol in dichloromethane to afford tert-butyl (S)-20-(4-(N-((2-amino-4-hydroxypteridin-6-yl)methyl)-2,2,2-trifluoroacetamido)benzamido)-4-(2-hydroxyethyl)-17-oxo-7,10,13-trioxa-4,16-diazahenicos-1-yn-21-oate (7) as colorless oil. Yield: 0.406 g, 51.37%; LCMS m/z 850.3 [M+1].

To a stirred solution tert-butyl (S)-20-(4-(N-((2-amino-4-hydroxypteridin-6-yl)methyl)-2,2,2-trifluoroacetamido)benzamido)-4-(2-hydroxyethyl)-17-oxo-7,10,13-trioxa-4,16-diazahenicos-1-yn-21-oate (7, 0.450 g, 0.529 mmol) in tetrahydrofuran (5.0 mL) was added Sat. aqueous sodium bicarbonate (5.0 mL). Reaction mixture was then stirred at room temperature for overnight. After completion, reaction mixture was concentrated under vacuum to obtained crude tert-butyl (S)-20-(4-(((2-amino-4-hydroxypteridin-6-yl)methyl)amino)benzamido)-4-(2-hydroxyethyl)-17-oxo-7,10,13-trioxa-4,16-diazahenicos-1-yn-21-oate (8) as yellow solid which was used as such for next step. Yield: 0.450 g, Crude. LCMS m/z 754.3 [M+1].

To a stirring solution tert-butyl (S)-20-(4-(((2-amino-4-hydroxypteridin-6-yl)methyl)amino)benzamido)-4-(2-hydroxyethyl)-17-oxo-7,10,13-trioxa-4,16-diazahenicos-1-yn-21-oate (8, 0.450 g, 0.579 mmol) in dichloromethane (4.5 mL, 70.3 mmol) was added trifluoroacetic acid (4.5 mL). Reaction mixture was then stirred at RT for 12 h. After completion, mixture was concentrated under vaccum and purified by prep HPLC (using 25-60% acetonitrile in water with 0.1% TFA) to get (S)-20-(4-(((2-amino-4-hydroxypteridin-6-yl)methyl)amino)benzamido)-4-(2-hydroxyethyl)-17-oxo-7,10,13-trioxa-4,16-diazahenicos-1-yn-21-oic acid (9) as yellow solid. Yield: 0.147 g, 35.29%. LCMS m/z 698.5 [M+1].

To a stirred solution of (S)-20-(4-(((2-amino-4-hydroxypteridin-6-yl)methyl)amino)benzamido)-4-(2-hydroxyethyl)-17-oxo-7,10,13-trioxa-4,16-diazahenicos-1-yn-21-oic acid (9, 0.050 g, 0.071 mol), and (R)-3-(1-((6-(2-(4-(2-(2-azidoethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (ISP2-438, 0.066 g, 2.5 eq., 0.179 mmol) in dimethyl sulfoxide (2 mL), was added Tetrakis(acetonitrile)copper(I) hexafluorophosphate (0.066 g, 2.5 eq., 0.179 mmol), and stirred at room temperature for 30 min. Thereafter, acetic acid (0.5 mL) was added and reaction mixture was diluted with acetonitrile and purified by prep HPLC (23-41% acetonitrile in water with 0.1% TFA). Fractions containing the desired product were combined and lyophilized to dryness to afford (S)-19-(4-(((2-amino-4-hydroxypteridin-6-yl)methyl)amino)benzamido)-3-((1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)methyl)-1-hydroxy-16-oxo-6,9,12-trioxa-3,15-diazaicosan-20-oic acid (Compound I-93) as an off white solid. Yield: 0.012 g, 12.44%; ¹H NMR (400 MHz, DMSO-d₆) δ8.94 (s, 2H), 8.63 (s, 1H), 8.10 (d, J=12.4 Hz, 1H), 8.03 (d, J=10.8 Hz, 2H), 7.88 (brs, 1H), 7.63 (d, J=16.8 Hz, 2H), 7.35 (dd, J=8.4 Hz, 1H), 6.96-6.89 (m, 2H), 6.63 (d, J=8.80 Hz, 2H), 6.33 (m, 1H), 4.55 (t, J=5.2 Hz, 2H), 4.47 (d, J=5.6 Hz, 2H), 4.24 (s, 3H), 3.90-3.81 (m, 6H), 3.74 (s, 2H), 3.55 (brs, 2H), 3.17-3.12 (m, 2H), 3.45 (m, 14H), 2.60 (s, 3H), 2.58 (s, 3H), 2.17 (t, J=6.0 Hz, 2H), 2.03-2.01 (m, 1H), 1.91-1.88 (m, 1H), 1.66 (d, J=6.0 Hz, 3H).

Compound I-94 was prepared by adapting the general method described above for compound I-93. The structure, names and data for Compound I-94 is provided below:

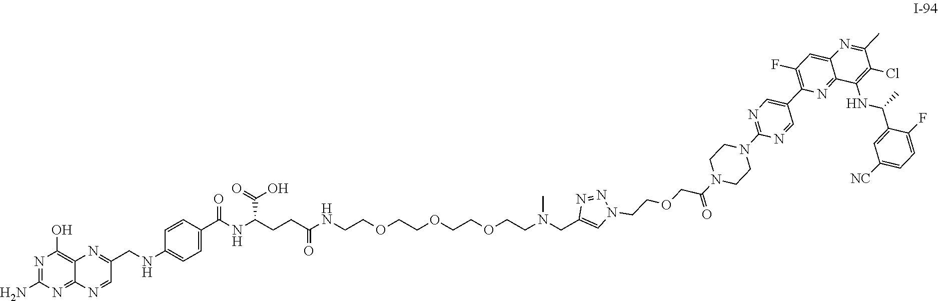 [SMILES: Cc1nc2cc(F)c(-c3cnc(N4CCN(C(=O)COCCn5cc(CN(C)CCOCCOCCOCCNC(=O)CC[C@H](NC(=O)c6ccc(NCc7cnc8nc(N)nc(O)c8n7)cc6)C(=O)O)nn5)CC4)nc3)nc2c(N[C@H](C)c2cc(C#N)ccc2F)c1Cl]

### (S)-18-(4-(((2-amino-4-hydroxypteridin-6-yl)methyl)amino)benzamido)-1-(1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)-2-methyl-15-oxo-5,811-trioxa-2,14-diazanonadecan-19-oic acid (Cpd. No. ISP2-501)

LCMS: m/z 1314.52 [M/2+1]⁺.

¹H NMR (400 MHz, DMSO-d6): δ12.5 (brs, 1H), 9.82 (brs, 1H), 8.92 (s, 2H), 8.65 (s, 1H), 8.36 (s, 1H), 8.17 (d, J=7.6 Hz, 1H), 8.12 (d, J=12.0 Hz, 1H), 8.03 (d, J=5.2 Hz, 1H), 7.86 (t, J=2.0 Hz 1H), 7.82-7.78 (m, 1H), 7.65 (d, J=8.8 Hz, 2H), 7.35 (t, J=9.6 Hz, 1H), 7.21-6.96 (m, 3H), 6.63 (d, J=8.8 Hz, 2H), 6.40 (t, J=8.0 Hz, 1H), 4.65 (t, J=4.4 Hz, 2H), 4.49-4.47 (m, 4H), 4.28 (s, 3H), 3.91 (t, J=4.8 Hz, 2H), 3.86 (bs, 4H), 3.76 (brs, 2H), 3.49-3.47 (m, 10H), 3.34 (t, J=6.4 Hz, 3H), 3.24-3.22 (m, 2H), 3.16 (t, J=5.6 Hz, 2H), 2.77 (s, 3H), 2.64 (m, 3H), 2.18 (t, J=7.6 Hz, 2H), 2.07-2.03 (m, 1H), 1.92-1.86 (m, 1H), 1.67 (d, J=6.8 Hz, 3H).

### 6.1.44. Example 44: Compound I-76

### (4S,4'S)-5,5′-((((12-(2-(2-(2-(3-oxo-3-(perfluorophenoxy)propoxy)ethoxy)ethoxy)ethyl)-3,6,9,15,18,21-hexaoxa-12-azatricosane-1,23-diyl)bis(1H-1,2,3-triazole-1,4-diyl))bis(butane-4,1-diyl))bis(azanediyl))bis(4-(4-(((2-amino-4-oxo-3,4-dihydropteridin-6-yl)methyl)amino)benzamido)-5-oxopentanoic acid) (Compound I-76)

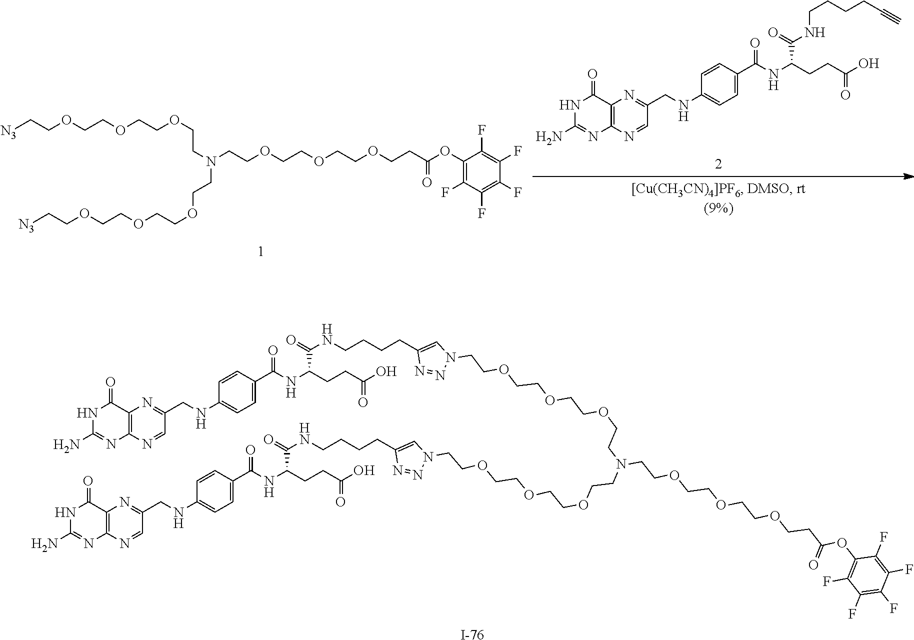

A solution of perfluorophenyl 1-azido-12-(2-(2-(2-(2-azidoethoxy)ethoxy)ethoxy)ethyl)-3,6,9,15,18,21-hexaoxa-12-azatetracosan-24-oate (1, 1.0 eq., 0.008 g, 0.010 mmol) and (S)-4-(4-(((2-amino-4-oxo-3,4-dihydropteridin-6-yl)methyl)amino)benzamido)-5-(hex-5-yn-1-ylamino)-5-oxopentanoic acid (2, 2.0 eq, 0.010 g, 0.020 mmol) in dimethylsulfoxide (0.3 mL) was stirred for 5 minutes. Then, tetrakis(acetonitrile)copper(I) hexafluorophosphate (5.6 eq., 0.021 g, 0.056 mmol) was added and reaction mixture was stirred at room temperature for 1 h. After completion, reaction mixture was diluted with acetonitrile and purified by prep HPLC (25-37% acetonitrile in water with 0.1% acetic acid). Fractions containing the desired product were combined and lyophilized to dryness to afford (4S,4'S)-5,5′-((((12-(2-(2-(2-(3-oxo-3-(perfluorophenoxy)propoxy)ethoxy)ethoxy)ethyl)-3,6,9,15,18,21-hexaoxa-12-azatricosane-1,23-diyl)bis(1H-1,2,3-triazole-1,4-diyl))bis(butane-4,1-diyl))bis(azanediyl))bis(4-(4-(((2-amino-4-oxo-3,4-dihydropteridin-6-yl)methyl)amino)benzamido)-5-oxopentanoic acid) (Compound I-76) as a yellow solid. Yield: 0.0022 g, 9.77%; LCMS m/z 1830.85 [M+1]⁺; ¹H NMR (400 MHz, DMSO-d₆) δ11.45 (bs, 1H), 8.63 (s, 1H), 7.92 (d, J=8.0 Hz, 2H), 7.83-7.81 (m, 2H), 7.76 (s, 2H), 7.64 (d, J=8.8 Hz, 4H), 7.20 (s, 2H), 7.07 (s, 2H), 6.95-6.92 (m, 5H), 6.62 (d, J=8.4 Hz, 4H), 4.47 (d, J=6.0 Hz, 4H), 4.44-4.41 (m, 4H), 4.33-4.31 (m, 2H), 3.77-3.69 (m, 8H), 3.53-3.46 (m, 36H), 3.05-2.99 (m, 6H), 2.60-2.56 (m, 3H), 2.30-2.20 (m, 4H), 2.00-1.95 (m, 2H), 1.88-1.83 (m, 2H), 1.57-1.53 (m, 4H), 1.43-1.39 (m, 4H).

### 6.1.45. Example 45: Compound I-77

### (S)-4-(4-(((2-amino-4-oxo-3,4-dihydropteridin-6-yl)methyl)amino)benzamido)-5-((4-(1-(39-(2,5-dioxo-2,5-dihydro-1H-pyrrol-1-yl)-37-oxo-3,6,9,12,15,18,21,24,27,30,33-undecaoxa-36-azanonatriacontyl)-1H-1,2,3-triazol-4-yl)butyl)amino)-5-oxopentanoic acid (Compound I-77)

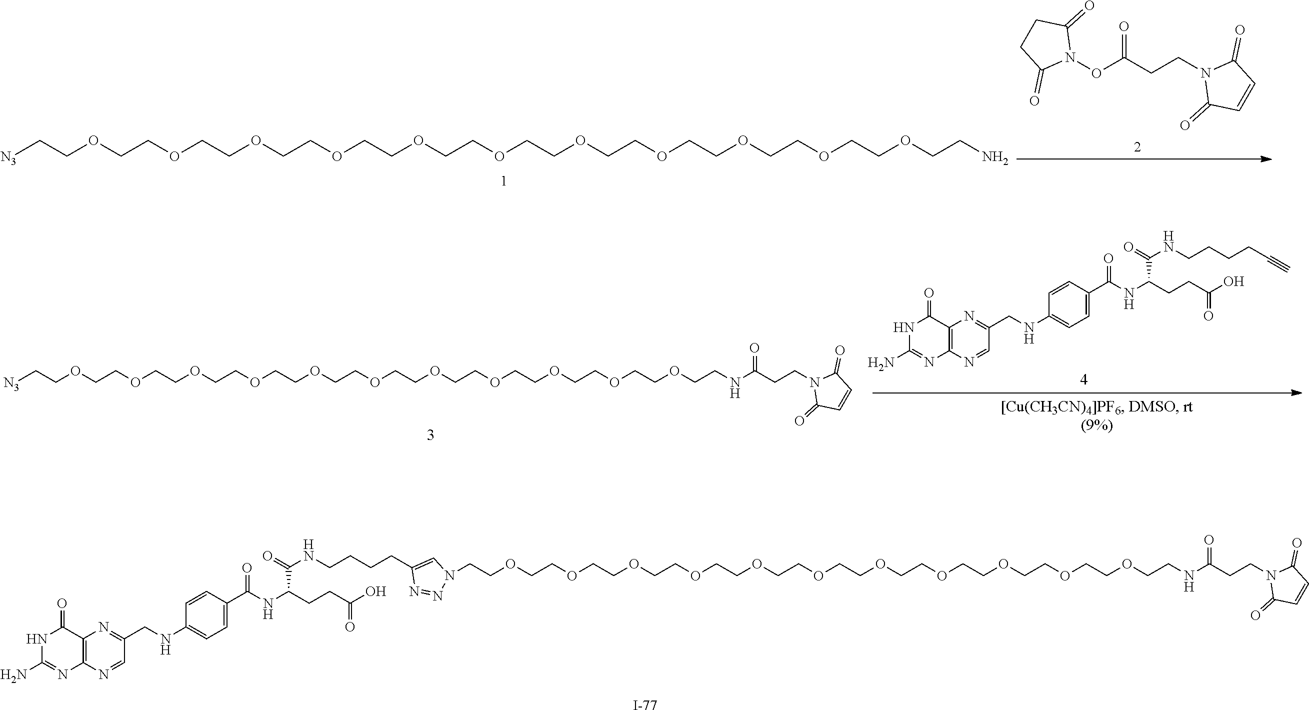

To a solution of 35-azido-3,6,9,12,15,18,21,24,27,30,33-undecaoxapentatriacontan-1-amine (1, 1.0 eq., 0.015 g, 0.026 mmol) in dimethylsulfoxide (0.5 mL), 2,5-dioxopyrrolidin-1-yl 3-(2,5-dioxo-2,5-dihydro-1H-pyrrol-1-yl)propanoate (2, 1.0 eq., 0.007 g, 0.026 mmol) was added and reaction mixture was stirred at room temperature for 1 h. Reaction was monitored by TLC. After completion of reaction, the solution of crude N-(35-azido-3,6,9,12,15,18,21,24,27,30,33-undecaoxapentatriacontyl)-3-(2,5-dioxo-2,5-dihydro-1H-pyrrol-1-yl)propanamide (3) was used as such for next reaction. To this solution, (S)-4-(4-(((2-amino-4-oxo-3,4-dihydropteridin-6-yl)methyl)amino)benzamido)-5-(hex-5-yn-1-ylamino)-5-oxopentanoic acid (4, 0.7 eq, 0.009 g, 0.018 mmol) was added and stirred for 5 minutes. Then, tetrakis(acetonitrile)copper(I) hexafluorophosphate (2.8 eq., 0.027 g, 0.073 mmol) was added and reaction mixture was stirred at room temperature for 1 h. After completion, reaction mixture was diluted with acetonitrile and purified by prep HPLC (23-40% acetonitrile in water with 0.1% acetic acid). Fractions containing the desired product were combined and lyophilized to dryness to afford (S)-4-(4-(((2-amino-4-oxo-3,4-dihydropteridin-6-yl)methyl)amino)benzamido)-5-((4-(1-(39-(2,5-dioxo-2,5-dihydro-1H-pyrrol-1-yl)-37-oxo-3,6,9,12,15,18,21,24,27,30,33-undecaoxa-36-azanonatriacontyl)-1H-1,2,3-triazol-4-yl)butyl)amino)-5-oxopentanoic acid (Compound I-77) as a yellow solid. Yield: 0.0042 g, 9.96%; LCMS m/z 1242.59 [M+1]⁺; ¹H NMR (400 MHz, DMSO-d₆) δ12.10 (bs, 1H), 8.63-8.62 (m, 1H), 8.02-7.94 (m, 2H), 7.83-7.81 (m, 1H), 7.76 (s, 1H), 7.65 (d, J=8.4 Hz, 2H), 7.00 (s, 1H), 6.93-6.91 (m, 1H), 6.62 (d, J=8.4 Hz, 2H), 4.44-4.42 (m, 4H), 4.32-4.31 (m, 1H), 3.78-3.75 (m, 2H), 3.65-3.56 (m, 2H), 3.49-3.48 (m, 44H), 3.32-3.31 (m, 2H), 3.16-3.11 (m, 2H), 3.06-3.04 (m, 2H), 2.60-2.50 (m, 2H), 2.34-2.30 (m, 2H), 2.30-2.20 (m, 2H), 1.96-1.94 (m, 1H), 1.86-1.82 (m, 1H), 1.56-1.51 (m, 2H), 1.45-1.38 (m, 2H).

### 6.1.46. Example 46: Compound I-95

### (S)-2-(4-(((2-amino-4-hydroxypteridin-6-yl)methyl)amino)benzamido)-5-(4-(2-(2-(2-((1-(2-(2-(4-(5-(7-chloro-8-(((R)-1-(5-cyano-2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol-4-yl)methoxy)ethoxy)ethoxy)ethyl)piperazin-1-yl)-5-oxopentanoic acid (Compound I-95)

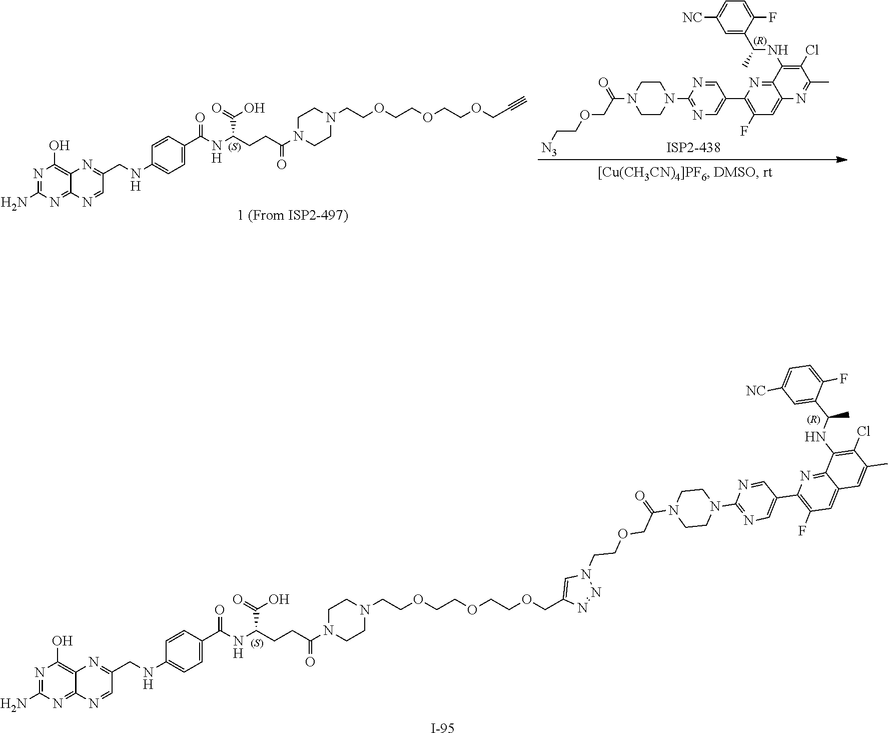

To a solution of (S)-2-(4-(((2-amino-4-hydroxypteridin-6-yl)methyl)amino)benzamido)-5-oxo-5-(4-(2-(2-(2-(prop-2-yn-1-yloxy)ethoxy)ethoxy)ethyl)piperazin-1-yl)pentanoic acid (1, 1.0 eq, 0.040 g, 0.058 mmol) and (R)-3-(1-((6-(2-(4-(2-(2-azidoethoxy)acetyl)piperazin-1-yl)pyrimidin-5-yl)-3-chloro-7-fluoro-2-methyl-1,5-naphthyridin-4-yl)amino)ethyl)-4-fluorobenzonitrile (ISP2-438, 1.0 eq, 0.038 g, 0.058 mmol) in dimethyl sulfoxide (1 mL), tetrakis(acetonitrile)copper(I) hexafluorophosphate (1.5 eq, 0.032 g, 0.088 mmol) was added and reaction mixture was stirred at room temperature for 1 h. After completion, reaction mixture was quenched with acetic acid (0.2 mL) and purified by prep HPLC (10-30% acetonitrile in water with 0.1% trifluoroacetic acid). Fractions containing the desired compound were combined and lyophilized to afford (S)-2-(4-(((2-amino-4-hydroxypteridin-6-yl)methyl)amino)benzamido)-5-(4-(2-(2-(2-((1-(2-(2-(4-(5-(7-chloro-8-(((R)-1-(5-cyano 2-fluorophenyl)ethyl)amino)-3-fluoro-6-methyl-1,5-naphthyridin-2-yl)pyrimidin-2-yl)piperazin-1-yl)-2-oxoethoxy)ethyl)-1H-1,2,3-triazol 4-yl)methoxy)ethoxy)ethoxy)ethyl)piperazin-1-yl)-5-oxopentanoic acid (Compound I-95) as a yellow solid. Yield: 0.031 g, 38.59%; LCMS m/z 664.65 [(M/2)+1]⁺; H NMR (400 MHz, DMSO-d6 with D₂O exchange) δ8.78 (s, 2H), 8.63 (s, 1H), 8.09 (s, 1H), 8.03 (d, J=11.2 Hz, 1H), 7.93 (d, J=11.2 Hz, 2H), 7.77-7.74 (m, 1H), 7.60 (d, J=8.8 Hz, 2H), 7.30 (t, J=9.6 Hz, 1H), 6.61 (d, J=8.8 Hz, 2H), 6.57-6.52 (m, 1H), 4.53-4.51 (m, 2H), 4.47 (d, J=6.8 Hz, 4H), 4.35-4.31 (m, 1H), 4.22 (s, 2H), 3.86 (t, J=4.4 Hz, 2H), 3.75-3.69 (m, 7H), 3.53-3.50 (m, 12H), 3.38 (bs, 2H), 3.24 (bs, 2H), 2.95-2.92 (m, 2H), 2.67 (s, 3H), 2.42 (s, 2H), 2.09-2.06 (m, 2H), 1.95-1.89 (m, 2H), 1.69 (d, J=6.8 Hz, 3H).

### 6.1.47. Example 47: Compound I-99

### (S)-2-(4-((2-(2-amino-4-oxo-3,4-dihydropteridin-6-yl)ethyl)amino)benzamido)hex-5-ynoic acid (Compound I-99)

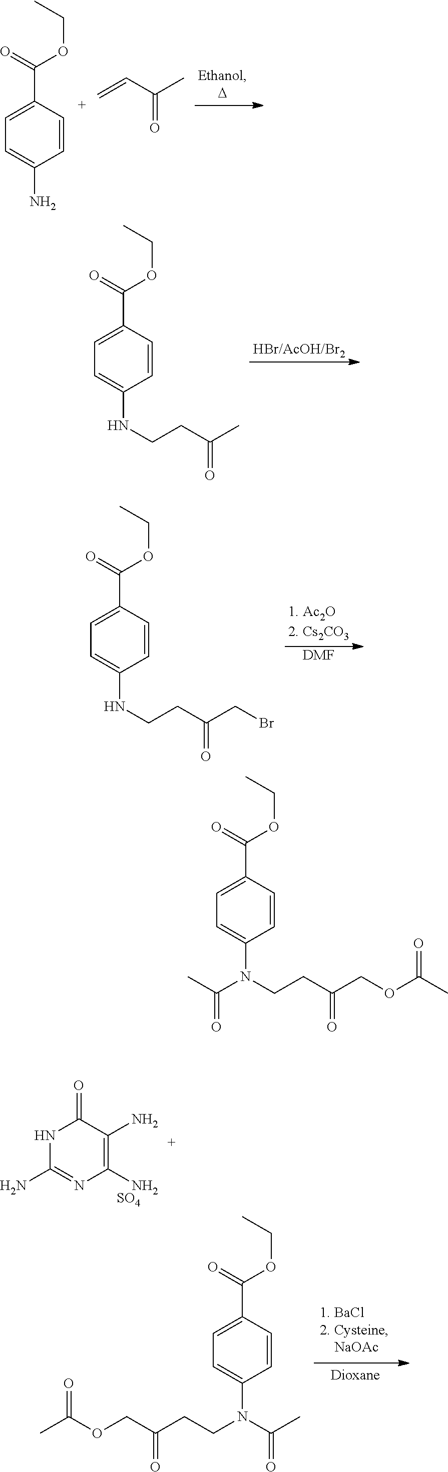

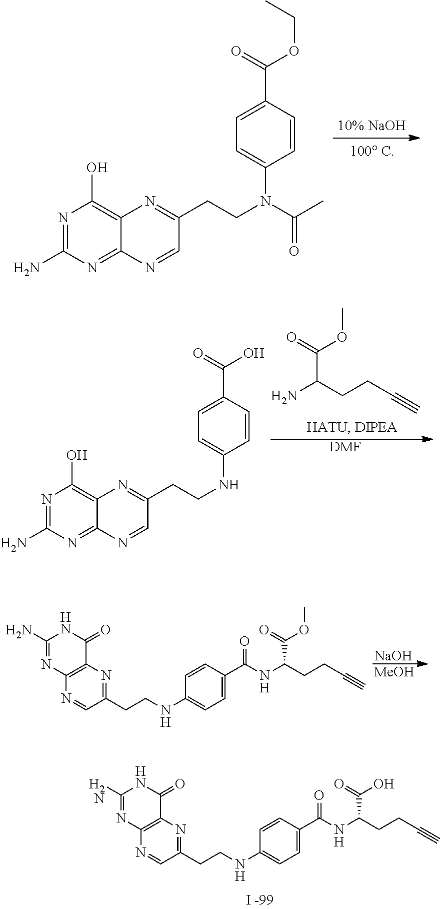

### Synthesis of ethyl 4-((3-oxobutyl)amino)benzoate

A solution of but-3-en-2-one (1.00 eq, 5.0 mL, 60.5 mmol) and ethyl 4-aminobenzoate (1.00 eq, 10.00 g, 60.5 mmol) in ethanol (20 mL) was heated to 60° C. for 18 hours. The reaction was cooled in an ice bath then filtered while rinsing with 3×5 mL cold ethanol to obtain ethyl 4-((3-oxobutyl)amino)benzoate as a white solid. Yield: 8.90 g, 62%.

### Synthesis of ethyl 4-((4-bromo-3-oxobutyl)amino)benzoate

A solution of ethyl 4-((3-oxobutyl)amino)benzoate (1.00 eq, 2.0 g, 8.50 mmol) in 33% HBr in glacial acetic acid (6.24 eq, 5.3 mL, 53.0 mmol) was treated with a solution of 33% v/v bromine in acetic acid (0.501 eq, 2.0 mL, 4.26 mmol). After 5 hours, the reaction was diluted with 20 volumes of diethyl ether (40 mL). The ether layer was decanted and the residue was further concentrated under vacuum. The residue was dissolved in 20 volumes of DCM (40 mL) then washed consecutively with saturated sodium bicarbonate (2×10 mL), brine, then dried over sodium sulfate, filtered and concentrated under reduced pressure. The residue was purified by silica gel chromatography (20-100% ethyl acetate in hexanes) to give ethyl 4-((4-bromo-3-oxobutyl)amino)benzoate. Yield: 1.00 g, 37%. LCMS: 313.9/315.9 [M+H]⁺.

### Synthesis of ethyl 4-(N-(4-acetoxy-3-oxobutyl)acetamido)benzoate

A solution of ethyl ethyl 4-((4-bromo-3-oxobutyl)amino)benzoate (1.00 eq, 850 mg, 2.15 mmol) in acetic anhydride (23.9 eq, 4857 μL, 51.4 mmol) was stirred at room temp for 2 hours before adding cesium carbonate (1.00 eq, 701 mg, 2.15 mmol). After 18 hours, the reaction was concentrated under high vacuum then diluted with DMSO and purified by reversed-phase HPLC (10-20-100% acetonitrile in water w/0.1% formic acid) to give ethyl 4-(N-(4-acetoxy-3-oxobutyl)acetamido)benzoate. Yield: 650 mg, 90%. LCMS: 336.1 [M+H]⁺.

### Synthesis of ethyl 4-(N-(2-(2-amino-4-hydroxypteridin-6-yl)ethyl)acetamido)benzoate

A slurry of 2,5,6-triaminopyrimidin-4(3H)-one sulfate hydrate (1.00 eq, 78.0 mg, 0.323 mmol) and ethyl 4-(N-(4-acetoxy-3-oxobutyl)acetamido)benzoate (1.03 eq, 112 mg, 0.333 mmol) in 23 volumes of water (1.79 mL) was stirred at 100° C. for 1 hour. The slurry was filtered and the ppt was washed with 12 volumes of water (0.900 mL). The combined filtrate was treated with L-cysteine hydrochloride (1.06 eq, 54.0 mg, 0.343 mmol) before adding 4 M aq. sodium acetate buffer (32.8 eq, 2.7 mL, 10.6 mmol). The pH was adjusted to -9.0 with triethylamine (4.44 eq, 0.20 mL, 1.43 mmol) then the reaction was purged with nitrogen and stirred at 60° C. for 18 hours. The solution was cooled to room temperature then the pH adjusted from −8 to 7 with 50 μL of 2 M aq. HCl. The slurry was then stored at 4° C. for 72 hours. The precipitate was filtered off and the filter cake washed with water and ether and the solids were dried under high vacuum to give ethyl 4-(N-(2-(2-amino-4-hydroxypteridin-6-yl)ethyl)acetamido)benzoate as a white solid. Yield: 82 mg, 64%. LCMS: 397.1 [M+H]⁺.

### Synthesis of sodium 4-((2-(2-amino-4-hydroxypteridin-6-yl)ethyl)amino)benzoate

A solution of ethyl 4-[acetyl-[2-(2-amino-4-hydroxy-pteridin-6-yl)ethyl]amino]benzoate (1.00 eq, 18.5 mg, 0.0467 mmol) in 10% sodium hydroxide (16.3 eq, 305 μL, 0.763 mmol) was stirred at 100° C. for 30 minutes. The reaction was then cooled to room temperature. The slurry was stored at −7° C. for 2 hours before filtering off crude product. The filter cake was dried under high vacuum to give sodium 4-((2-(2-amino-4-hydroxypteridin-6-yl)ethyl)amino)benzoate. Yield: 10 mg, 62%. LCMS: 327.1 [M+H]⁺.

### Synthesis of methyl (S)-2-(4-((2-(2-amino-4-oxo-3,4-dihydropteridin-6-yl)ethyl)amino)benzamido)hex-5-ynoate

A mixture of sodium 4-((2-(2-amino-4-hydroxypteridin-6-yl)ethyl)amino)benzoate (1.00 eq, 17.0 mg, 0.0488 mmol), methyl (S)-2-aminohex-5-ynoate hydrochloride (2.00 eq, 17.3 mg, 0.0976 mmol) and N,N-diisopropylethylamine (2.00 eq, 17 μL, 0.0976 mmol) in DMF (0.9762 mL) was treated with HATU (1.10 eq, 20.4 mg, 0.0537 mmol). After 20 minutes, the reaction was diluted with DMSO, acidified with formic acid then purified by reversed-phase HPLC (10-20-45% acetonitrile in water w/0.1% formic acid) to give methyl (S)-2-(4-((2-(2-amino-4-oxo-3,4-dihydropteridin-6-yl)ethyl)amino)benzamido)hex-5-ynoate. Yield: 16 mg, 73%. LCMS: 450.2 [M+H]⁺.

### Synthesis of (2S)-2-[[4-[2-(2-amino-4-oxo-3H-pteridin-6-yl)ethylamino]benzoyl]amino]hex-5-ynoic acid

A solution of methyl (S)-2-(4-((2-(2-amino-4-oxo-3,4-dihydropteridin-6-yl)ethyl)amino)benzamido)hex-5-ynoate (1.00 eq, 20.0 mg, 0.0445 mmol) in methanol (0.445 mL) was treated with 1 M sodium hydroxide (3.00 eq, 0.13 mL, 0.133 mmol). After several hours, the reaction was acidified with TFA then purified by reversed-phase HPLC (5-40% acetonitrile in water w/0.1% TFA) to give the title compound, (2S)-2-[[4-[2-(2-amino-4-oxo-3H-pteridin-6-yl)ethylamino]benzoyl]amino]hex-5-ynoic acid, as a white solid. Yield: 17 mg, 85%. LCMS: 436.2 [M+H]⁺.

### 6.2. Conjugation Examples

### 6.2.1. Example 48: Conjugation of Isothiocyanate-Based Ligand-Linker Compounds with Antibodies

This example provides a general protocol for the conjugation of the isothiocyanate-based ligand-linker compounds described herein with the primary amines on lysine residues of the antibodies described herein.

The antibody is buffer exchanged into 100 mM sodium bicarbonate buffer pH 9.0 at 5 mg/mL concentration, after which about 10-30 equivalents of the isothiocyanate-based ligand-linker compound (freshly prepared as 20 mM stock solution in DMSO) is added and incubated overnight at ambient temperature in a tube revolver at 10 rpm.

The conjugates containing on average eight ligand-linker moieties per antibody are purified using a PD-10 desalting column (GE Healthcare) and followed with formulating the final conjugate into PBS pH 7.4 with Amicon Ultra 15 mL Centrifugal Filters with 30 kDa molecular weight cutoff.

### 6.2.2. Example 49: Conjugation of Perfluorophenoxy-Based Ligand-Linker Compounds with Antibodies

This example provides a general protocol for the conjugation of the perfluorophenoxy-based ligand-linker compounds described herein with the primary amines on lysine residues of the antibodies described herein.

The antibody is buffer exchanged into 50 mM sodium phosphate buffer pH 8.0 at 5 mg/mL concentration, after which about 22 equivalents of perfluorophenoxy-based ligand-linker compound (freshly prepared as 20 mM stock solution in DMSO) is added and incubated for 3 hours at ambient temperature in a tube revolver at 10 rpm.

The conjugates containing on average eight ligand-linker moieties per antibody are purified using a PD-10 desalting column (GE Healthcare) and followed with formulating the final conjugate into PBS pH 7.4 with Amicon Ultra 15 mL Centrifugal Filters with 30 kDa molecular weight cutoff.

### 6.2.3. Example 50: Determination of Drug to Antibody Ratio (DAR) by Mass Spectrometry

This example provides the method for determining DAR values for the conjugates prepared as described in Examples 41 and 42. To determine the DAR value, 10 g of the antibody (unconjugated or conjugated) is treated 2 μL of non-reducing denaturing buffer (10×, New England Biolabs) for 10 minutes at 75° C. The denatured antibody solution is then deglycosylated by adding 1.5 μL of Rapid-PNGase F (New England Biolabs) and incubated for 10 minutes at 50° C. Deglycosylated samples are diluted 50-fold in water and analyzed on a Waters ACQUITY UPLC interfaced to Xevo G2-S QToF mass spectrometer. Deconvoluted masses are obtained using Waters MassLynx 4.2 Software. DAR values are calculated using a weighted average of the peak intensities corresponding to each loading species using the formula below:

$$D A R = \sum ( drug load distribution ( \% ) of each Ab with drug load n ) ( n ) / 100$$

### 6.2.4. Example 51: Determination of Purity of Conjugates by SEC Method

Purity of the conjugates prepared as described in Examples 48 and 49 are determined through size exclusion high performance liquid chromatography (SEC-HPLC) using a 20 minute isocratic method with a mobile phase of 0.2 M sodium phosphate, 0.2 M potassium chloride, 15 w/v isopropanol, pH 6.8. An injection volume of 10 μL is loaded to a TSKgel SuperSW3000 column, at a constant flow rate of 0.35 mL/min. Chromatographs are integrated based on elution time to calculate the purity of monomeric conjugate species.

### 6.2.5. Example 52: Antibody Disulfide Reduction and Ligand-Linker Conjugation to Antibody

This example provides an exemplary protocol for reduction of the disulfides of the antibodies described herein, and conjugation of the reduced antibodies to the ligand-linker compounds described herein.

### Protocol:

### Antibody Disulfide Reduction

A) Dilute antibody to 15 mg/mL (0.1 mM IgG) in PBS, pH 7.4.
B) Prepare a fresh 20 mM (5.7 mg/mL) stock solution of tris(2 carboxyethyl)phosphine (TCEP) in H₂O.
C) Add 25 μL of TCEP stock solution from step B) above to 1 mL of antibody from step A) above (0.5 mM final concentration TCEP).
D) Incubate at 37° C. for 2 hours (check for free thiols using 5,5′-dithiobis-(2-nitrobenzoic acid) (DTNB) test).
E) Aliquot the reduced antibody into 4 tubes (250 μL each).

### Ligand-Linker Conjugation to Antibody

A) Prepare 10 mM stock solution of ligand-linker compound in DMSO (DMA, DMF or CH₃CN are also acceptable).
B) Add 5 equivalents of 12.5 μL stock solution from step A) above to each tube of reduced antibody (0.5 mM final concentration ligand-linker compound stock solution).
C) Incubate overnight at 4° C. for 4 hours at room temperature; check for free thiols using DTNB test.
D) Run analytical hydrophobic interaction chromatography (HIC) to determine DAR and homogeneity.

### Biological Examples

### 6.2.6. Example 53: Folate Receptor α and Folate Receptor β KO Generation

This example provides the protocol for generation of folate receptor knockout (KO) cells. Cells are washed with PBS and detached using TrypLE. Media is added to the flask to deactivate trypsin. Cells are collected and counted. A total of 1×10⁶ cells are then centrifuged at 300×g for 5 minutes. The cell pellet is washed once with PBS and centrifuged at 300×g for 5 minutes. The cell pellet is resuspended in Lonza SE buffer supplemented with supplement 1 and electroporation enhancer (5 μM final). CRISPR RNP reaction begins by combining equal volumes of 100 μM crRNA and tracrRNA in a PCR tube. Using a thermocycler, this mixture is heated to 95° C. for 5 minutes and allowed slowly to cool to room temperature. Using sgRNA specific for either folate receptor α or folate receptor β, the annealed sgRNA product is combined with TrueCut Cas9 and allowed to incubate at RT for 15 minutes. Resuspended cells in SE buffer are mixed with the RNP reaction and allowed to incubate for 5 minutes. The entire reaction contents is then placed in a single well of a 16-well electroporation cuvette. Using a Lonza Amaxa cells are pulsed with code CA-163. After pulsing, cells are plated into a 10 cm dish. Six days post-RNP, a portion of cells are collected and lysates are prepared to test for knock-out by western.

### 6.2.7. Example 54: Alexa Fluor 647 Conjugation

Antibodies are conjugated to Alexa Fluor 647 using Alexa Fluor™ 647 Protein Labeling Kit (Invitrogen) per the manufacturer's protocol. In brief, antibodies to be labeled are diluted to 2 mg/mL in PBS to a total volume of 500 μL. A 15 DOL (degree of labeling) is used for the conjugation with the fluorophore. Free dye is removed by pre-wetting an Amicon 30 kDa filter with PBS. After incubation, the conjugation reaction is then added to the filter and spun at high speed for 10 minutes. Retained solution is then resuspended in PBS to a final volume of 1 mL and stored at 4° C. indefinitely.

### 6.2.8. Example 55: Live-Cell Surface Staining by Flow Cytometry

This example provides a protocol for the determination of the effect of the conjugates described herein on EGFR levels measured by surface staining using flow cytometry.

Hela parental or folate receptor knockout cells are plated in 6 well plates and treated with vehicle (PBS), unconjugated antibody (e.g., anti-EGFR), or the conjugates prepared as described in Examples 48 and 49 for the indicated period of time.

After incubation, media is aspirated and cells are washed three times with PBS, lifted using Accutase and pelleted by centrifugation at 300×g for 5 minutes. Cells are resuspended in cold FACS buffer and kept cold for the remainder of the staining procedure. A portion of cells are excluded from staining procedure as an unstained control. Cells are stained with either human IgG Isotype-AF647 or cetuximab-AF647 conjugates for 1 h on ice in the dark. Cells are then spun at 300×g for 5 min at 4° C. and washed with cold FACS buffer for a total of three washes. After the final wash, cells are resuspended in 100 μL of FACS buffer with DAPI added at a final concentration of 5 μg/mL. Cells are analyzed using a BioRad ZE5 flow cytometer and data is analyzed using Flowlo software. Cells are first gated to remove debris, doublets and dead cells (DAPI negative). EGFR cell surface levels are determined based on AF647 mean fluorescence intensity (MFI).

### 6.2.9. Example 56: Measurement of Total EGFR Levels by Traditional Western Blotting

This example provides the protocol for the measurement of the time course activity of the conjugates prepared as described in Examples 48 and 49 on total EGFR levels in Hela parental and folate receptor KO cells measured by traditional Western blotting.

Once all time-points are collected, all cell pellets are resuspended in 50 μL of radioimmunoprecipitation assay (RIPA) buffer (+protease/phosphatase inhibitor+nuclease).

Lysates are incubated on ice for 1 h.

Lysates are then spun at high-speed for 10 min at 4° C.

40 μL of cleared lysate is transferred to a 96 well plate.

All lysate concentrations are calculated using BCA assay (1:3 dilution).

All lysates are equalized to 2 mg/mL using RIPA as diluent.

Equal volumes (15 μL) of lysate are then mixed with LDS sample buffer (3×LDS+2.5× reducing agent).

Samples are incubated at 98° C. for mins and allowed to cool.

Samples are vortexed and spun down.

15 μL of sample is loaded onto a 26-well bis-tris 4-12% midi-gel.

Gel is allowed to run at 180V for 20 mins.

Gels are transferred to nitrocellulose membrane using iBlot 2 (20V constant, 7 mins).

Membranes are washed 1× in PBS and then placed in Odyssey blocking buffer for 1 h RT with shaking.

Primary antibodies mouse anti-β-actin (SCB) and rabbit anti-EGFR (CST) are diluted 1:1000 in blocking buffer and allowed to incubate overnight at 4° C. with shaking.

Membranes are washed thrice with PBS-T (Tween20 0.1%), at least 5 mins each wash.

Secondary antibodies anti-mouse 680rd and anti-rabbit 800 cw are diluted 1:5000 in blocking buffer and allowed to incubate for 1 h at RT with shaking.

Membranes are washed thrice with PBS-T (Tween20 0.1%), at least 5 mins each wash.

Membranes are imaged using licor odyssey scanner.

### 6.2.10. Example 57: Measurement of Cellular EGFR Protein Levels Evaluated by Immunocytochemistry

This example provides an exemplary protocol for the determination of the effect of the conjugates prepared as described in Examples 48 and 49 on cellular EGFR protein levels evaluated by immunocytochemistry.

Hela parental or folate receptor knockout cells are plated in 6 well plates and treated with vehicle (PBS), unconjugated antibody, or the conjugates prepared as described in Examples 41 and 42 at 37° C. for 24 hours. After incubation, media is aspirated and cells are washed thrice with PBS. Cells are fixed with 4% PFA for 10 minutes at room temperature, washed three times with PBS and then blocked with 5% BSA in PBS for 1 hour at RT. Cells are permeabilized with 0.2% Triton-X100 in PBS for 15 minutes. After washing, cells are stained with goat anti-EGFR (AF321; R&D Systems) in blocking buffer overnight at 4 C. After washing, cells are stained with anti-goat 800 CW secondary or CellTag700, and imaged on Licor scanner.

### 6.3. Biology Methods

### 6.3.1. Example 58: SPR Binding Kinetics of Subject Compounds to Folate Receptor

A Biacore 8K+ was used to measure the association and dissociation rates of example compounds to FOLR2 (Acro Biosystems, Cat: F02-H5223-100 μg). Briefly, FOLR2 was reconstituted according to the manufacture's instructions to 200 μg/mL. Both the active and reference surfaces of a Cytiva CM5 Series S Sensor Chip were activated with a 1:1 mixture of 400 mM EDC and 100 mM NHS for 420 s at 10 μL/min. 25 μg/mL FOLR2 diluted in Sodium Acetate pH 5.5 was injected on the active channel (Flow Cell 2) for 420 s at 10p/min. Both the reference and active surfaces were then capped with 1M ethanolamine, pH 8.5 for 420 s, achieving an immobilization level of ˜4200 RU. Example compounds were serially diluted 1:1 in running buffer (10 mM HEPES, 150 mM NaCl, 0.05% T20, pH 7.5, 2% DMSO) from 100 nM to 3.125 nM and flowed over the active and reference surfaces for 120 s, and then allowed to dissociate in running buffer for 600 s, at 30 uL/min. The FOLR2 immobilized active surface and the reference surface were regenerated with a 30 s pulse of Sodium Acetate pH 5.5 at 30 μL/min. Sensorgrams were solvent corrected, double-referenced, and the data fitted and analyzed in Biacore Insight Evaluation Software Version 3.0.11.15423.

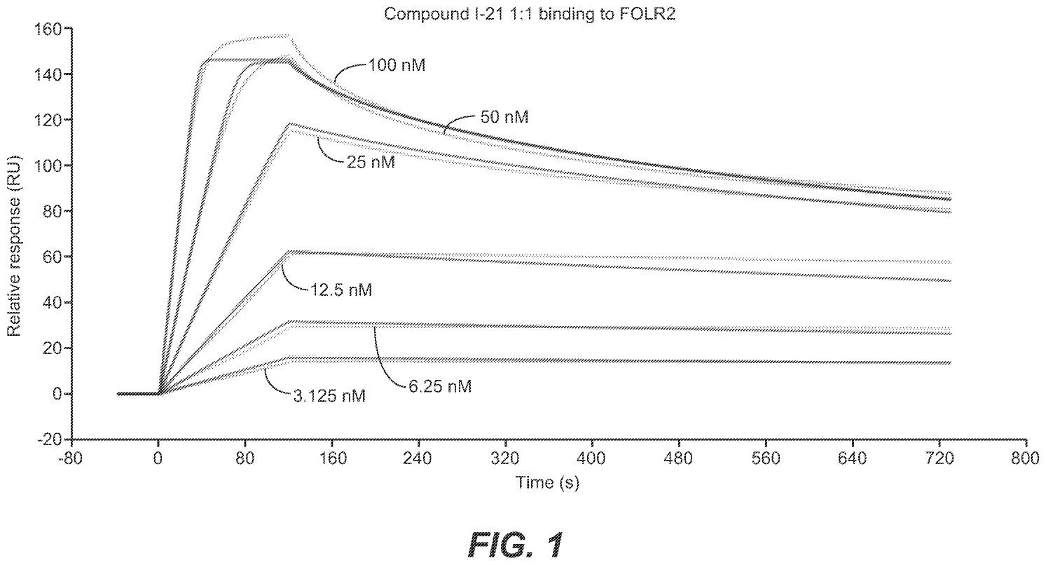

FIG. 1 illustrates the SPR sensorgrams for various concentrations of compound I-21 (3.125 nM to 100 nM) to folate receptor 2 (FOLR2), illustrating 1:1 binding.

TABLE 10
SPR results of compound I-21 biding to FOLR2

| | | Ligand | Amount | | |
|---|---|---|---|---|---|
| | Immobilized | MW | Immobilized | Analyte 1 | MW |
| Channel | ligand | (Da) | (RU) | Solution | (Da) |
| 1 | FOLR2 | 32500 | 4213.2 | Example # | 1218.7 |

| Kinetics Chi² | ka | kd | KD | Rmax | | Expected Rmax | |
|---|---|---|---|---|---|---|---|
| (RU²) | (1/Ms) | (1/s) | (M) | (RU) | tc | (RU) | Stoichiometry |
| 7.10E+00 | 5.57E+06 | 4.64E−03 | 8.33E−10 | 147.5 | 1.47E+07 | 158.0 | 0.9 |

### 6.3.2. Example 59: SPR Binding Kinetics of Subject Compounds to TNFα Trimer

Streptavidin 100 μg/mL Streptavidin (Invitrogen Cat #: 434302) diluted in 10 mM sodium acetate, pH 4.5) was immobilized to both the reference and active surfaces as described in Example 51. The surfaces were conditioned with three injections of running Buffer (10 mM HEPES, 150 mMNaCl, 0.05% T20, pH 7.5, 2% DMSO) at a flow rate of 10 μL/min, Contact time: 60 s. Flow rate: 10 μL/min. The active surface was treated with biotinylated linked TNFα trimer 20 μg/mL diluted in running buffer at a flow rate of 5 μL/min contact time: 600 s, yielding a final response of ˜3000 RU. Example compounds were then serially diluted 1:1 in running buffer (10 mM HEPES, 150 mM NaCl, 0.05% T20, pH 7.5, 2% DMSO) from 30 uM to 937.5 nM and flowed over the active and reference surfaces from lowest concentration to greatest concentration in the single-cycle kinetics format, and then allowed to dissociate in running buffer for 7200 s, at 30 μL/min. Sensorgrams were solvent corrected, double-referenced, and the data fitted and analyzed in Biacore Insight Evaluation Software Version 3.0.11.15423.

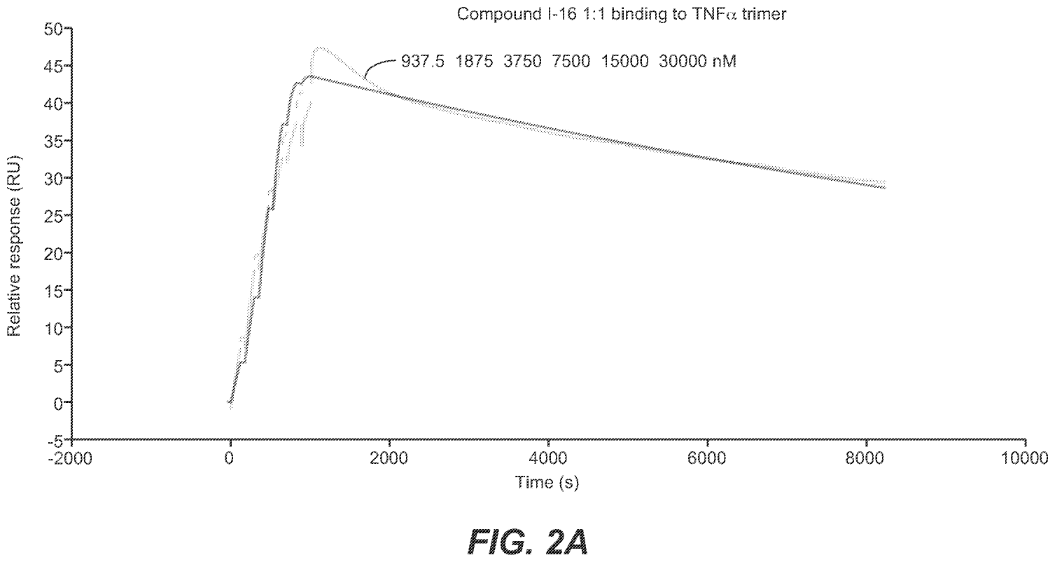

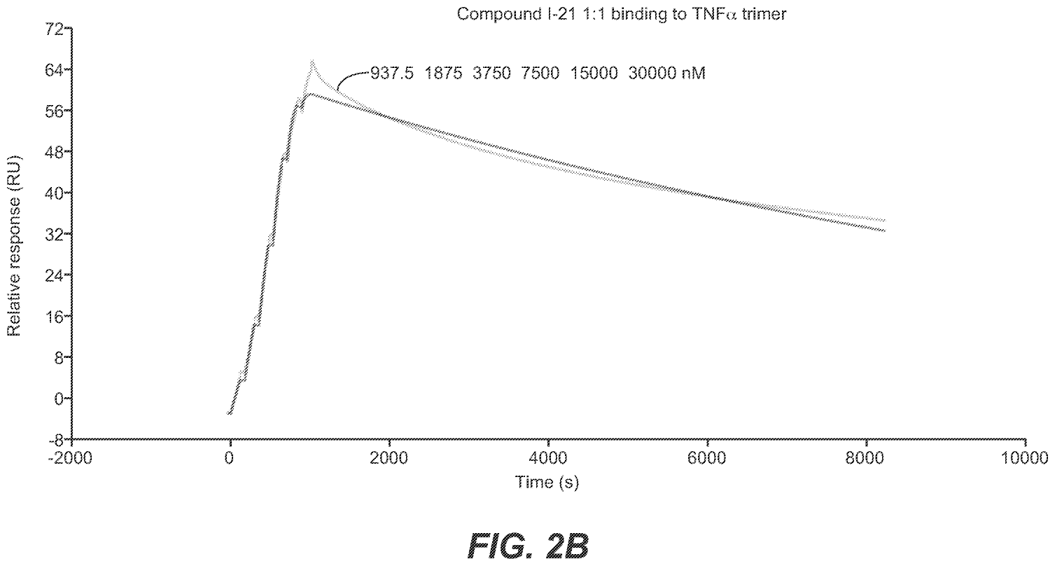

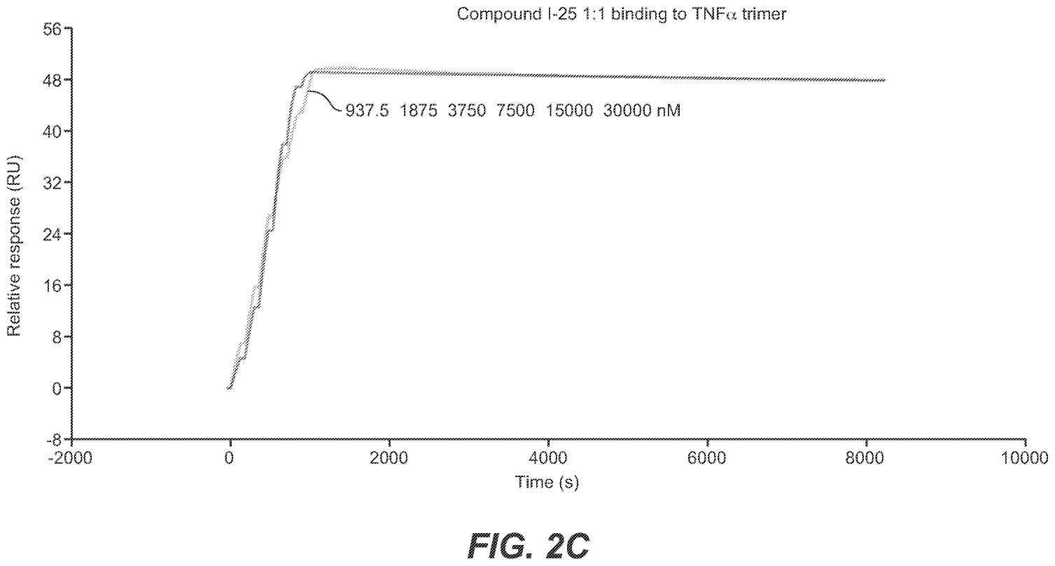

FIG. 2A illustrates the SPR sensorgrams for compound 1-16 binding to TNF-alpha trimer, illustrating 1:1 binding.

TABLE 11
SPR results of compound I-16 biding to TNF-alpha trimer

| | | Ligand | Amount | Single cycle | Analyte |
|---|---|---|---|---|---|
| | | MW | Immobilized | kinetics 1 | MW |
| Channel | Immobilized ligand | (Da) | (RU) | Solution | (Da) |
| 6 | (TNFalpha trimer) | 53729.8 | 2847.5 | Example # | 1130.5 |

| Kinetics Chi² | ka | kd | KD | Rmax | | Expected Rmax | |
|---|---|---|---|---|---|---|---|
| (RU²) | (1/Ms) | (1/s) | (M) | (RU) | tc | (RU) | Stoichiometry |
| 2.50E+00 | 1.16E+03 | 5.81E−05 | 5.02E−08 | 43.7 | 9.74E+09 | 59.9 | 0.7 |

FIG. 2B illustrates the SPR sensorgrams for compound 1-21 binding to TNF-alpha trimer, illustrating 1:1 binding.

TABLE 12
SPR results of compound I-21 biding to TNF-alpha trimer

| | | Ligand | | Single cycle | Analyte |
|---|---|---|---|---|---|
| | | MW | Amount | kinetics 1 | MW |
| Channel | Immobilized ligand | (Da) | Immobilized | Solution | (Da) |
| 3 | (TNFalpha trimer) | 53729.8 | 2960.9 | | 1218.7 |

| Kinetics Chi² | ka | kd | KD | Rmax | | Expected Rmax | |
|---|---|---|---|---|---|---|---|
| (RU²) | (1/Ms) | (1/s) | (M) | (RU) | tc | (RU) | Stoichiometry |
| 1.76E+00 | 9.63E+02 | 7.75E−05 | 8.05E−08 | 62.3 | 2.62E+09 | 67.2 | 0.9 |

FIG. 2C illustrates the SPR sensorgrams for compound 1-25 binding to TNF-alpha trimer, illustrating 1:1 binding.

TABLE 13
SPR results of compound I-25 biding to TNF-alpha trimer

| | | Ligand | | Single cycle | Analyte |
|---|---|---|---|---|---|
| | | MW | Amount | kinetics 1 | MW |
| Channel | Immobilized ligand | (Da) | Immobilized | Solution | (Da) |
| 4 | (TNFa trimer) | 53729.8 | 3023.6 | Example # | 1322.4 |

| Kinetics Chi² | ka | | | Rmax | | Expected Rmax | |
|---|---|---|---|---|---|---|---|
| (RU²) | (1/Ms) | kd (1/s) | KD (M) | (RU) | tc | (RU) | Stoichiometry |
| 9.56E−01 | 8.74E+02 | 3.65E−06 | 4.18E−09 | 49.3 | 1.10E+10 | 74.4 | 0.7 |

### 6.3.3. Example 60: SPR Co-Engagement of Subject Compound-TNFα Trimer Complex to Folate Receptor

The active (Flow cell 2) surfaces of a Cytiva CM5 Series S Sensor Chip functionalized with FOLR2 as described in Example 58. Example compounds were diluted to 10 nM with 50 and 100 nM TNFα single chain trimer or 150 nM and 300 nM TNFα native monomer in running buffer (10 mM HEPES, 150 mM NaCl, 0.05% T20, pH 7.5, 2% DMSO), allowed to incubate for 5 hours at 25° C. and then flowed over the active and reference surfaces for 110 s, and then allowed to dissociate in running buffer for 600 s, at a flow rate of 30 μL/min. The FOLR2 immobilized active surface and the reference surface were regenerated with a 30 s pulse of 4M MgCl₂ at 30 μL/min then allowed to equilibrate 240 s. Sensorgrams were solvent corrected, double-referenced, and the data fitted and analyzed in Biacore Insight Evaluation Software Version 3.0.11.15423.

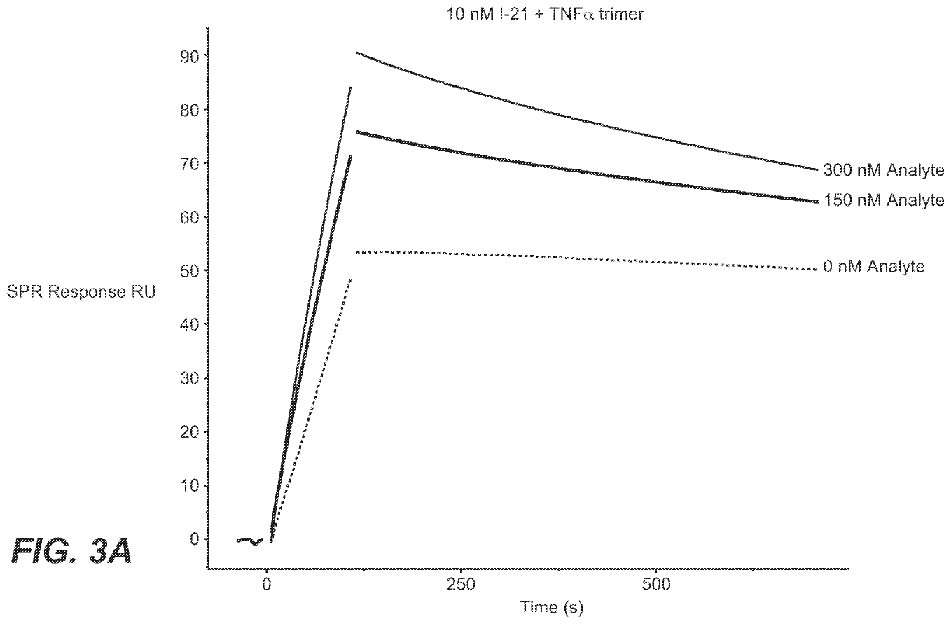

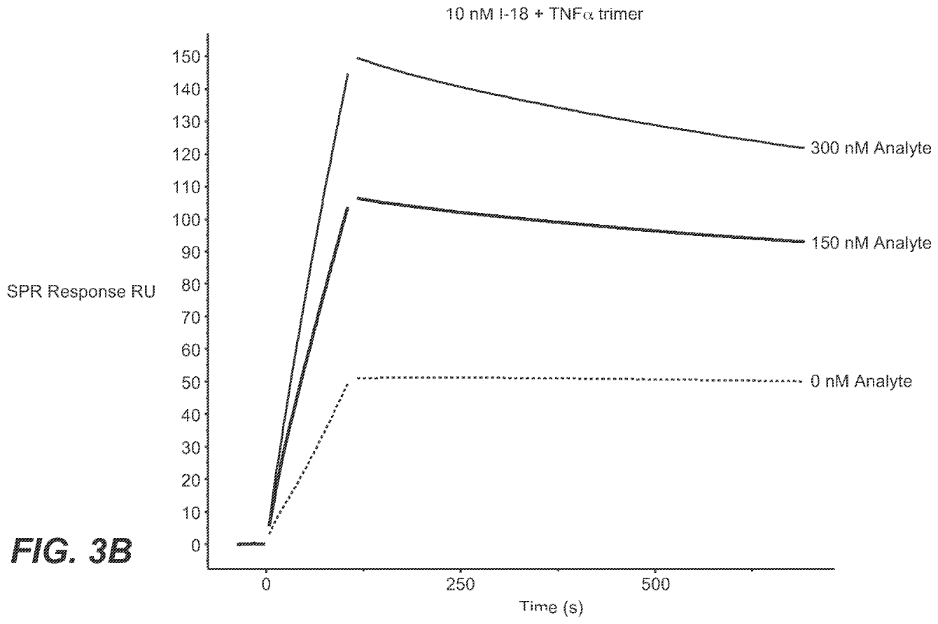

FIG. 3A-3B show SPR co-engagement of compound (I-21) TNFα trimer complex to folate receptor (FIG. 3A); and compound (1-18) TNFα trimer complex to folate receptor (FIG. 3B).

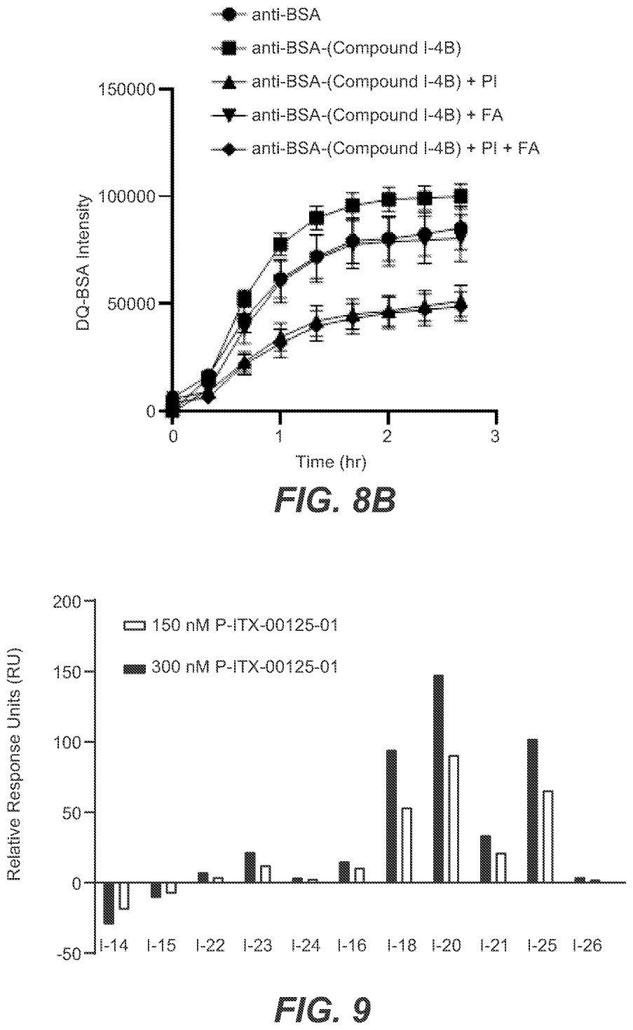

FIG. 9 shows complex formation observed on SPR for example compounds (TNFα and 10 nM compound binding FOLR2).

### 6.3.4. Example 61: Subject Compound Mediated Uptake by TNFα in THP-1 Cells

THP-1 cells, a monocytic leukemia cell line that endogenously express folate receptor beta, were used as a model system to measure example compound stimulated uptake of in-house produced TNFα conjugated to the pH sensitive fluorescent dye pHrodo green (ThermoFisher Scientific #P35369).

THP-1 cells were maintained in RPMI (Gibco #61870143) with 10% v/v FBS (VWR #89510-188) with 2 mM L-alanyl-L-glutamine dipeptide, 100 units/ml penicillin and 100 μg/ml streptomycin (Gibco #15140148). Cells were pelleted, resuspended in folate-free RPMI (Gibco #27016021) and seeded at 50,000 cells per well of a 96 well plate. Example compound was added to each well as a log 2 dilution series ranging from 20 μM to 125 nM final concentration. Cells were incubated for 24 hrs and washed/resuspended in phosphate buffered saline (pH 7.4) with 1% w/v BSA. Uptake was measured using a Novocyte Advanteon Flow Cytometer (Agilent Technologies) with the 488 nM laser and FITC detection configuration.

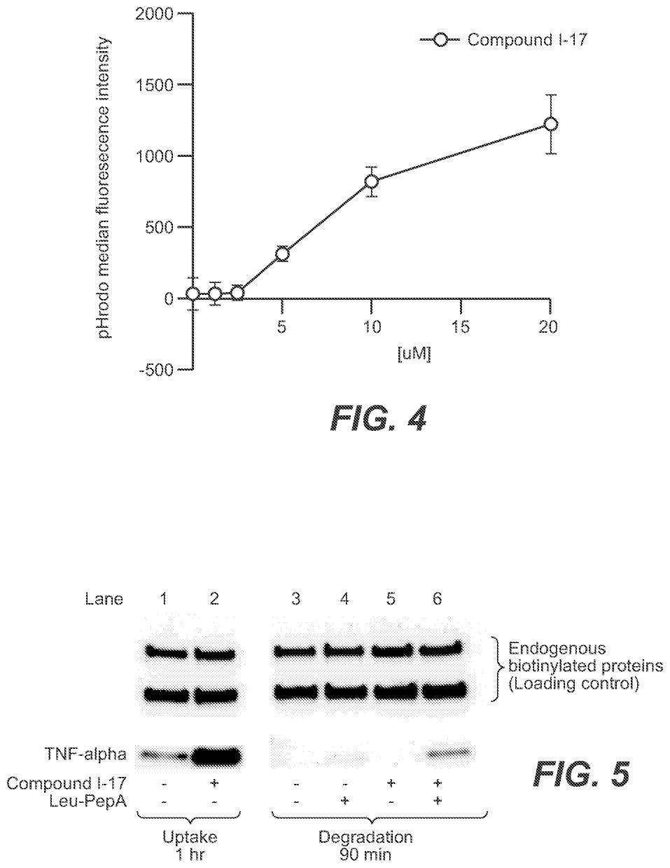

As shown in FIG. 4, treatment with an example compound stimulated uptake of TNFα-pHrodo in a dose-dependent manner as measured by median fluorescence intensity of pHrodo dye.

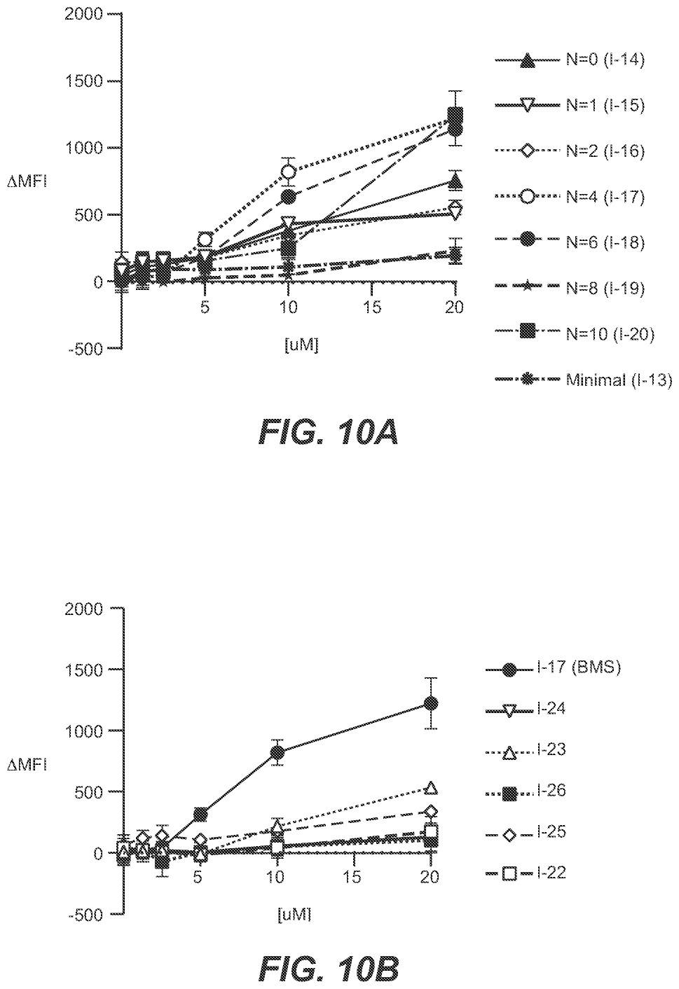

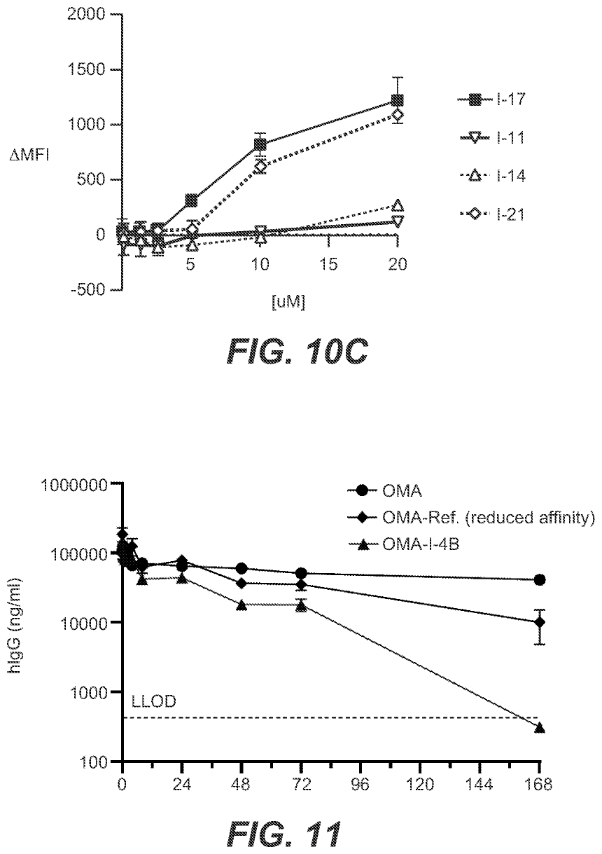

FIG. 10A shows compound stimulated uptake of TNFα for compounds having various linker lengths (where “N” represents the number of ethylene glycol moieties in the linker chain). FIG. 10B shows compound stimulated uptake of TNFα for compounds with various TNFα binding moieties (“Y”). FIG. 10C shows that compounds (1-21) and (1-17) (i.e., example compounds of formula (IIIA) and (IIIIB) respectively) both stimulate uptake of TNFα.

A summary of compound stimulated uptake of TNFα in THP1 cells, along with the compounds TNFα and folate (FOLR2) binding data is provided in Table 14.

TABLE 14
Example compound binding, and TNFα uptake data

| | | | Uptake in THP1 cells | | |
|---|---|---|---|---|---|
| Compound | TNFα binding | Folate binding | (run) | | |
| # | SPR | SPR Kd | 2 | 3 | 4 |
| (I-13) | A | — | + | ++ | ++ |
| (I-14) | A | 0.2 nM | + | ++ | − |
| (I-15) | A | 0.3 nM | − | +++ | ++ |
| (I-16) | A | 0.3 nM | +++ | +++ | ++ |
| (I-17) | B | — | ++ | ++ | +++ |
| (I-21) | A | 0.8 nM | | +++ | +++ |
| (I-18) | A | 0.4 nM | +++ | ++ | +++ |
| (I-19) | — | — | + | − | + |
| (I-20) | B | 0.2 nM | +++ | + | ++ |
| (I-22) | C | 0.3 nM | + | + | + |
| (I-23) | B | 0.3 nM | | ++ | ++ |
| (I-24) | C | 0.4 nM | | + | + |
| (I-25) | A | 0.5 nM | + | +++ | ++ |
| (I-26) | C | 0.2 nM | +++ | + | + |

Where: A = less than 100 nM, B = less than 1 μM, and C = less than 5 μM; where + = ΔMFI less than that of I-17; ++ = ΔMFI ≈ to that of I-17; and +++ = ΔMFI greater than that of I-17 comparisons of uptake at10 μM.

### 6.3.5. Example 62: Subject Compound Mediated Degradation of TNFα in THP-1 Cells

To measure degradation of internalized TNFα, a pulse-chase experiment was performed whereby cells were treated with +/− example compounds and/or +/− protease inhibitors and incubated for 1 hr in media containing TNFα-biotin to allow uptake and then washed and incubated 90 minutes in fresh media without TNFα to allow degradation. Samples were collected after the pulse phase (60 min) and chase phase (90 min) to measure uptake and degradation, respectively.

THP-1 cells were seeded at 100,000 cells per well of 12 well plate with 1 mL RPMI+10% w/w FBS+1× penicillin/streptomycin and differentiated using 20 ng/mL phorbol myristate acetate (Sigma-Aldrich #P8139) for 72 hrs. After 72 hrs media was replaced with 1 ml RPMI+10% v/v FBS+1× penicillin/streptomycin and TNFα-biotin (AcroBiosystems #50-201-9879) was added to 12.5 nM final concentration. Where noted example compound was added at 10 μM final concentration and protease inhibitors were added at 100 μg/ml final concentration for leupeptin (Sigma-Aldrich #L2884) or 1 μg/ml final concentration for pepstatin A (Sigma-Aldrich #BP26715). While cysteine protease inhibitors such as leupeptin alone have been used to inhibit lysosomal proteases, degradation early in the endocytic pathway in macrophages is initiated by the endosome resident aspartyl protease Cathepsin D that is inhibited by pepstatin A (Diment, J B C, 1985; Diment, J B C, 1988). The plate was incubated for 1 hr at 37° C. in with 5% CO₂. After 1 hr the plate was transferred to ice and washed 5× with cold phosphate buffered saline (pH 7.4). Cells for +/− example compound conditions were lysed in well with 50 ul RIPA buffer (Pierce #89900)+1 mM MgCl₂+25 U/ml benzonase (Sigma-Aldrich #E1014) for 10 min on ice. Lysates were frozen at −80° C. as the uptake samples. 1 ml pre-warmed RPMI+10% FBS+1× penicillin/streptomycin was added to each well and plates were incubated for 90 min at 37° C. in 5% CO₂. The plate was transferred to ice and washed 3× with cold phosphate buffered saline (pH 7.4). Cells were lysed by adding 50 ul RIPA+1 mM MgCl₂+25 U/ml benzonase and incubating on ice for 10 min. Lysates were frozen at −80° C. as the degradation samples. 3.66 ug of each sample was loaded onto to a 4-20% TGX pre-cast gradient gel (BioRad #4561093DC) and separated at 200V for 1 hr using a Mini-PROTEAN gel electrophoresis system (BioRad). Proteins were transferred to PVDF membrane using Transblot Turbo (BioRad) at 1.3 amps for 10 min. The membrane was blocked in tris-buffered saline (pH 7.4) with 5% w/v BSA for 2 hr and incubated overnight at 4° C. with Neutravidin-HRP (ThermoFisher #31001) at 0.4 μg/ml in tris-buffered saline (pH 7.4) with 5% w/v BSA. The blot was developed with ECL Plus Western Detection Substrate (Pierce #32132) and imaged using the ChemiDoc MP Imaging System (BioRad). Relative quantification of bands was performed using the ImageLab software (BioRad) with endogenously biotinylated proteins serving as endogenous controls.

As shown in FIG. 5, treatment with example compounds stimulated the uptake of TNFα during the pulse phase (lane 1 compared to lane 2). After the chase phase, the pool of internalized TNFα was undetectable in the absence of protease inhibitors (lanes 3 and 5), consistent with degradation of internalized TNFα occurring within 90 min. Partial rescue of TNFα level when uptake is in the presence of leupeptin and pepstatin A was consistent with degradation occurring within the endolysosomal pathway (lane 2 compared to lane 6).

### 6.3.6. Example 63: Compound Mediated Depletion of TNFα from the Medium of THP-1 Cells

An in-house recombinantly expressed and purified N-terminal fusion of HiBiT with TNFα was used to measure example compound-stimulated depletion from the media. HiBiT and largeBiT are split nanoluciferase polypeptides that result in complementation of luciferase activity in the presence of both polypeptides. The level of HiBit-TNFα in the media over time can be measure by sampling the media and mixing with the Nano-Glo HiBiT Extracellular Detection Reagent (Promega Corporation) containing the largeBiT polypeptide and nanoluciferase substrate.

THP-1 cells were seeded at 100,000 cells per well of 12 well plate with 1 mL RPMI+10% w/w FBS+1× penicillin/streptomycin and differentiated using 20 ng/mL phorbol myristate acetate (Sigma-Aldrich #P8139) for 72 hrs. After 72 hrs the growth medium was replaced with 1 ml RPMI+10% v/v FBS+1× penicillin/streptomycin containing 12.5 nM HiBit-TNFα. Where noted example compound was added at 10 μM final concentration. The medium was sampled (50 l) at 0, 24, 48 and 96 hr and luciferase activity was measured using the Nano-Glo HiBiT Extracellular Detection Reagent.

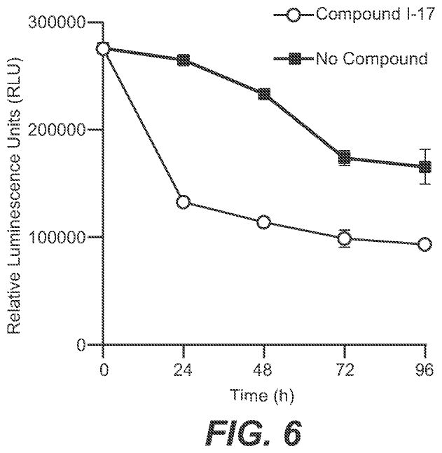

As shown in FIG. 6, treatment with an example compound (1-17) resulted in more rapid depletion of HiBiT-TNFα over the first 24 hours and greater total depletion of HiBiT-TNFα from the culture medium.

### 6.3.7. Example 64: Compound Mediated Uptake of Target Protein IgE in THP-1 Cells

Assessment of a folate receptor-mediated pathway for internalization of a target protein via an exemplary antibody/folate receptor-binding conjugate was performed. An exemplary folate receptor ligand—omalizumab conjugate was generated by non-specific conjugation to lysine residues of in-house produced omalizumab anti-IgE antibody with pentafluorophenyl ester compound (I-4B) N-2-(4-(((2-amino-4-oxo-3,4-dihydropteridin-6-yl)methyl)amino)benzoyl)-N5-(4-(1-(27-oxo-27-(perfluorophenoxy)-3,6,9,12,15,18,21,24-octaoxaheptacosyl)-1H-1,2,3-triazol-4-yl)butyl)-L-glutamine. The conjugate produced yielded an average ligand/antibody ratio (e.g., “DAR”) of 3.71.

IgE (BioRad #HCA171G) was labelled with Alexa-647 (Invitrogen #A²⁰¹⁷³) according to manufacturer's instructions to produce a fluorescently labelled target protein (IgE) with degree of labeling of 8.25. Uptake of the labelled IgE-Alexa647 was evaluated in a cell-based competition cell uptake assay using wild type (WT) THP-1 cells and folate receptor 2 (FOLR2)-overexpressing THP-1 cells. The THP-1 cells overexpressing FOLR2 were generated by stable transduction with lentivirus encoding FOLR2-IRES-GFP. THP-1 WT and FOLR2 overexpressing cells were maintained in RPMI (Gibco #61870143) with 10% v/v FBS (VWR #89510-188) with 2 mM L-alanyl-L-glutamine dipeptide, 100 units/ml penicillin and 100 μg/ml streptomycin (Gibco #27016021). Stable FOLR2 over-expression cells were mixed at a 50:50 ratio with WT THP-1 cells and seeded in a 96 well plate at 50,000 cells in 100 μl folate-free RPMI (Gibco #61870143) with 10% v/v FBS, 2 mM L-alanyl-L-glutamine dipeptide, 100 units/ml penicillin and 100 μg/ml streptomycin. In a separate 96 well plate, IgE-Alexa647 was mixed with unmodified omalizumab or the exemplary omalizumab-folate receptor ligand conjugate at a concentration of 600 nM each in 100 μl folate-free RPMI+10% FBS+2 μM non-targeting human IgG antibody (to block background binding due to Fc receptor expression in THP-1 cells) and incubated at room temperature for 30 mins. The immune complex was serially diluted 2-fold to produce a dose response range from 300 nM-9.375 nM. The IgE/FR ligand-omalizumab conjugate immune complex mixture was transferred to the plate containing a 50:50 mixture of THP-1 WT and FOLR2 over expressing cells and incubated for 2 h at 37° C. with 5% CO₂.

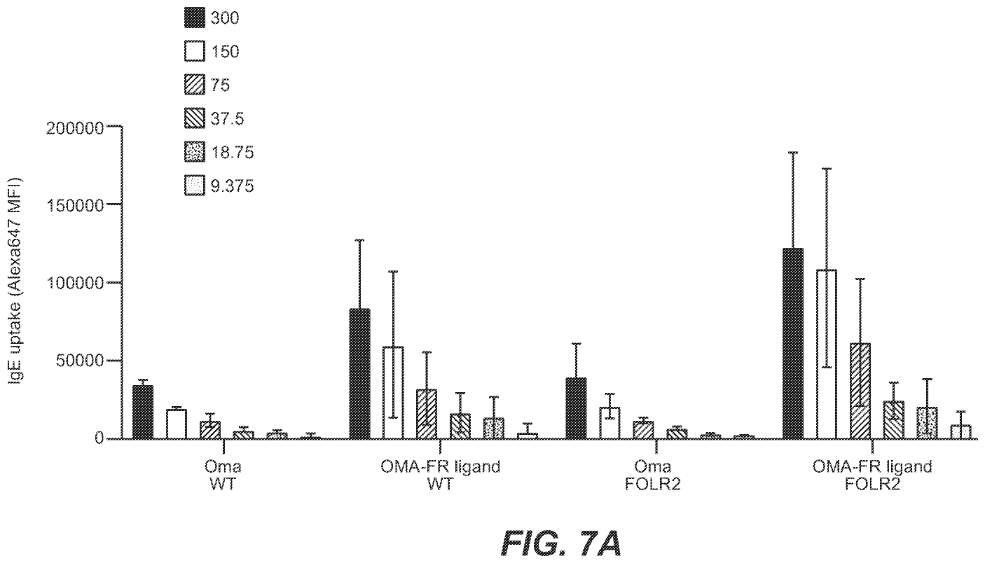

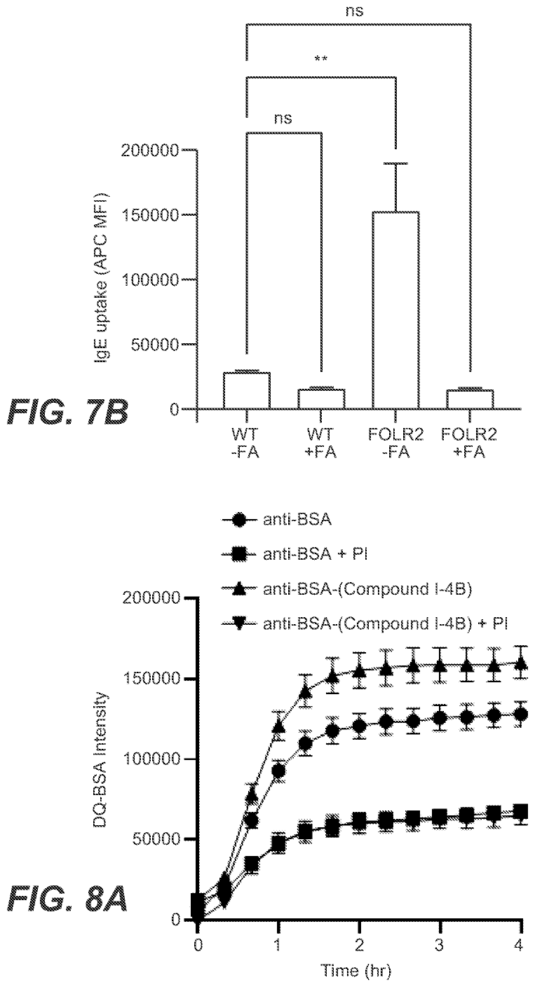

Uptake in WT and FOLR2 overexpressing cells was evaluated using flow cytometry by comparing the median fluorescence intensity of IgE-Alexa647 in the GFP negative (WT) and GFP positive (FOLR2) populations. As shown in FIG. 7A, uptake of IgE-Alexa647 was enhanced across the dose range in both the WT and FOLR2 over expressing cells with the exemplary omalizumab-folate receptor ligand conjugate as compared to unmodified omalizumab control.

To demonstrate that the enhanced IgE-Alexa647 uptake observed with the exemplary conjugate is dependent on folate receptor, the competition experiment, as described above, was performed with 100 nM omalizumab-folate receptor ligand conjugate in the presence or absence of excess folic acid (2 μM). Results were analyzed using one-way ANOVA with multiple comparisons with THP-1 WT and no folic acid condition as the control. As shown in FIG. 7B, increased uptake (p=0.0055) was observed with the exemplary conjugate in FOLR2 overexpressing THP-1 cells compared to WT cells, and addition of folic acid decreased uptake to the WT level. Taken together, these results demonstrated folate receptor dependent uptake of IgE using the omalizumab-folate receptor ligand conjugate.

### 6.3.8. Example 65: Degradation of Target Protein IgE Using an Exemplary Antibody-Folate Receptor Ligand Conjugate in PMA Differentiated THP-1 Cells

To determine that conjugate-mediated cell uptake leads to degradation of target protein the fate of a self-quenched fluorescently labeled target protein was monitored. DQ-BSA-FL (Invitrogen #D12050) is bovine serum albumin (BSA) conjugated with BODIPY dye with a high degree of labeling to produce a self-quenched fluorescent BSA reagent. Upon proteolysis within the endolysosomal pathway of the cell, dequenching of the BODIPY fluorophores results in the appearance of bright fluorescence which can be monitored by live cell imaging. Since DQ-BSA is generally used as a reagent to monitor endocytosis, it was expected that complexing DQ-BSA with anti-BSA antibody conjugated to folate receptor ligand of this disclosure would enhance cell uptake of DQ-BSA, and degradation would be inferred by increased signal that is diminished when treatment was performed in the presence of endolysomal protease inhibitors leupeptin (Sigma-Aldrich #L2884) and pepstatin A (Sigma-Aldrich #BP26715).

Folate receptor ligand-conjugated anti-BSA was generated by non-specific conjugation to lysine residues of anti-BSA antibody (Invitrogen #A¹¹¹³³) with Compound I-4B. To monitor uptake and degradation, 250,000 THP-1 cells were seeded into a 12 well tissue culture treated plate and differentiated to macrophage-like cells using 20 ng/ml phorbol myristate acetate (Sigma-Aldrich #P8139) for 72 hrs. PMA increases FOLR2 expression (Samaniego, 2020) and facilitates THP-1 live cell imaging by adopting an adherent phenotype upon differentiation. Cells were rinsed 3 times with PBS and 1 ml folate-free RPMI with no serum was added to each well. 100 nM of anti-BSA antibody with or without folate conjugation, 100 nM DQ-BSA and 1 μM of non-targeting antibody was added to each well in the presence or absence of pepstatin A (1 μg/ml) and leupeptin (10 μM) The plate was immediately transferred to the Incucyte live cell imaging system and imaged every 20 m for 4 h. As shown in FIG. 8A, in the presence of protease inhibitors (PI), the intracellular fluorescent signal was diminished. These results demonstrate that the exemplary conjugate enhanced uptake of DQ-BSA, and resulted in proteolysis and dequenching of BODIPY dye in the endolysosomal pathway.

To confirm folate receptor dependence, the experiment above was repeated in the presence or absence of folic acid (2 μM) and/or pepstatin A (1 μg/ml) and leupeptin (10 μM). Images were collected using the Incucyte live cell imaging system every 20 m for 2 h 40 m. As shown in FIG. 8B, in the presence of folic acid (FA), the fluorescent signal of anti-BSA control antibody without folate (anti-BSA) was the same as anti-BSA conjugate with the folate receptor ligand (anti-BSA/Compound I-4B). These results indicate that the stimulation of uptake and degradation of target protein DQ-BSA with the exemplary conjugate is folate receptor-mediated.

### 6.3.9. Example 66: Inhibition of IL-6 Induced by TNFα

TNF signaling leads to the induction of serum IL-6, which can be blocked with biologics such as etanercept (Enbrel). Eight-week-old female C57BL/6 are purchased from Charles River Labs and allowed to acclimate for one week prior to start of study. After the acclimation period, mice are ear tagged, weighed, and orally dosed with vehicle or 3, 10, or 30 mg/kg of example compound or dosed by intraperitoneal (IP) injection with 10 mg/kg positive control (mouse Enbrel). One hour later, mouse TNF-α is administered by intravenous (IV) injection. Two hours after mouse TNF-α treatment, blood is collected via cardiac puncture to measure for serum mouse IL-6 by ELISA from R&D systems.

### 6.3.10. Example 67: Efficacy in Mouse Model of Rheumatoid Arthritis Induced by Collagen Antibodies

Collagen antibodies can be used to induce rheumatoid arthritis in mice. Eight-week-old female BALB/c are purchased from Charles River Labs and allowed to acclimate for one week prior to start of study. After the acclimation period, mice are ear tagged, weighed, and dosed by intraperitoneal (IP) injection with a mixture of four monoclonal anti-mouse type II collagen antibodies at 1 mg per antibody. Three days after antibody administration, 10 g of lipopolysaccharide (E. coli O111:B4) from Sigma are dosed IP and six hours after lipopolysaccharide injection, vehicle or 3, 10, or 30 mg/kg of example compound is orally dosed twice a day. As a positive control, 10 mg/kg of mouse Enbrel is dosed SC twice a week until the end of the study. Mice are weighed and monitored daily for the development and severity of paw inflammation. The development and severity of paw inflammation is measured by two methods. The first method to measure paw inflammation is to visually evaluate the paws and score based on the severity of inflammation/swelling of the digits and paws. The clinical score is based on the following numbering system: (1) one or more swollen digits per paw; (2) mild paw swelling; (3) moderate paw swelling; (4) fusion of joints/ankylosis with a maximum score of 16 per mouse. The second method to measure paw inflammation is to use the plethysmometer from World Precision Instruments to measure each paw.

### 6.3.11. Example 68: Efficacy in Human Transgenic TNFα Mouse Model of Rheumatoid Arthritis

Human transgenic TNF-α mice express the human TNFα transgene, which develop severe chronic arthritis of the forepaws and hindpaw by approximately 20 weeks of age. Twelve week old female human transgenic TNFα mice are purchased from Taconic and allowed to acclimate for one week prior to start of study. After the acclimation period, mice are ear tagged, weighed, and orally dosed with vehicle or 3, 10, or 30 mg/kg of example compound is orally dosed twice a day or 10 mg/kg of a positive control (mouse Enbrel) is dosed SC twice a week until the end of the study. Mice are weighed and monitored daily for the development and severity of paw inflammation. The development and severity of paw inflammation is measured by two methods. The first method to measure the development and severity of paw inflammation is to visually evaluate the paws and score based on the severity of inflammation/swelling of the digits and paws. The clinical score is based on the following numbering system: (1) one or more swollen digits per paw; (2) mild paw swelling; (3) moderate paw swelling; (4) fusion of joints/ankylosis with a maximum score of 16 per mouse. The second method to measure the development and severity of paw inflammation is to use the plethysmometer from World Precision Instruments to measure each paw.

### 6.3.12. Example 69: Efficacy in Mouse Model of Colitis Induced by Dextran Sodium Sulfate

Dextran Sodium Sulfate is used to induce colitis in mice. Eight-week-old female C₅₇BL/6 are purchased from Charles River Labs and allowed to acclimate for one week prior to start of study. After the acclimation period, mice are ear tagged, weighed, and allowed to drink regular drinking water or 3% DSS in drinking water for the duration of the study. One week after switching the drinking water, vehicle or 3, 10, or 30 mg/kg of example compound is orally dosed twice a day or 10 mg/kg of a positive control (mouse Enbrel) is dosed SC twice a week until the end of the study. Mice are weighed and monitored daily for the development and severity of colitis. The development and severity of colitis is measured by a clinical score composed of body weight, rectal bleeding, and diarrhea and histological score of the colon.

### 6.3.13. Example 70: LYTAC Mediated Uptake of TNFα Using Hetero-Bifunctional Small Molecules Targeting Folate Receptor in THP-1 Cells

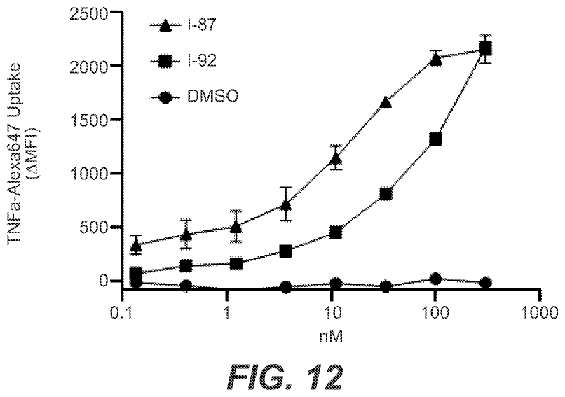

Hetero-bifunctional small molecule LYTACs consisting of a TNFα binding moiety, linker and folate receptor binding moiety were tested to determine if folate receptor can internalize TNFα. THP-1 cells overexpressing FOLR2 were generated by stable transduction with lentivirus encoding FOLR2-IRES-GFP. THP-1 WT and FOLR2 overexpressing cells were maintained in RPMI (Gibco #61870143) with 10% v/v FBS (VWR #89510-188) with 2 mM L-alanyl-L-glutamine dipeptide, 100 units/ml penicillin and 100 μg/ml streptomycin (Gibco #27016021). Stable FOLR2 over-expression cells were mixed at a 50:50 ratio with WT THP-1 cells and seeded in a 96 well plate at 50,000 cells in 100 μl folate-free RPMI (Gibco #61870143) with 10% v/v FBS, 2 mM L-alanyl-L-glutamine dipeptide, 100 units/ml penicillin and 100 μg/ml streptomycin. In a separate 96 well plate, TNFα/folate hetero-bifunctional small molecules were mixed with unmodified TNFα conjugated with Alexa647 by non-specific lysine chemistry. Small molecule LYTACs at a concentration of 600 nM (2× final concentration) in 100 μl folate-free RPMI+10% FBS were incubated at room temperature for 30 mins. The immune complex was serially diluted 3-fold to produce a dose response range from 300 nM-0.137 nM (final concentration). The TNFα/LYTAC complex mixture was transferred to the plate containing a 50:50 mixture of THP1 WT and FOLR2 over expressing cells and incubated for 2 h at 37° C. with 5% C₀₂. Uptake in WT and FOLR2 overexpressing cells was evaluated by flow cytometry by comparing the median fluorescence intensity of IgE-Alexa647 in the GFP negative (WT) and GFP positive (FOLR2) populations and was quantified as the difference between the mean fluorescence intensities in the APC channel for the GFP positive and GFP negative populations (ΔMFI). As shown in FIG. 12 compounds I-87 and I-92 both showed a dose-dependent uptake of TNFα compared the no compound control (DMSO) with compound I-87 showing greater potency (EC50=15.29 nM). These results demonstrate that hetero-bifunctional molecules targeting TNFα and folate receptor beta are sufficient to mediate LYTAC-dependent internalization of TNFα.

### TNFα Binding Assay

TNFα binding was measured in black 96-well plates using a time-resolved FRET assay using a previously described fluorescence acceptor TNFα probe (Xiao et al J. Med. Chem. 2020). Biotinylated TNFα was purchased from Acro Biosystems. Binding reactions were conducted in 20 mM Hepes (pH 7.5) 10 mM MgCl2 0.015% Tween-20 0.05 mg/ml BSA 2% DMSO with 1 nM TNFα 0.5 nM Streptavidin-Tb and 80 nM probe. Test compounds were resuspended in DMSO and 3-fold serial dilutions were made at 100× final concentrations. Fluorescence was measured using λex=340 nm, λem=490, 520 nm on an SpectraMax M5e plate reader (Molecular Devices) after 16 hr incubation time. Dose responses were conducted in duplicate and normalized to the response with DMSO (high) and 100 nM (R)-3-(1-((3-chloro-7-fluoro-6-(2-(2-hydroxypropan-2-yl)pyrimidin-5-yl)-2-methyl-1,5-naphthyridin-4-yl)amino)ethyl)-4-fluorobenzonitrile (low) on each plate. IC50 values were determined by fitting to 4-parameter curves in GraphPad Prism.

### Folate Receptor Binding Assay

Folate receptor binding was measured in black 96-well plates using a fluorescence polarization assay. A fluorescent probe consisting of a folate analog linked to Cy5 (FR-Cy) was custom synthesized.

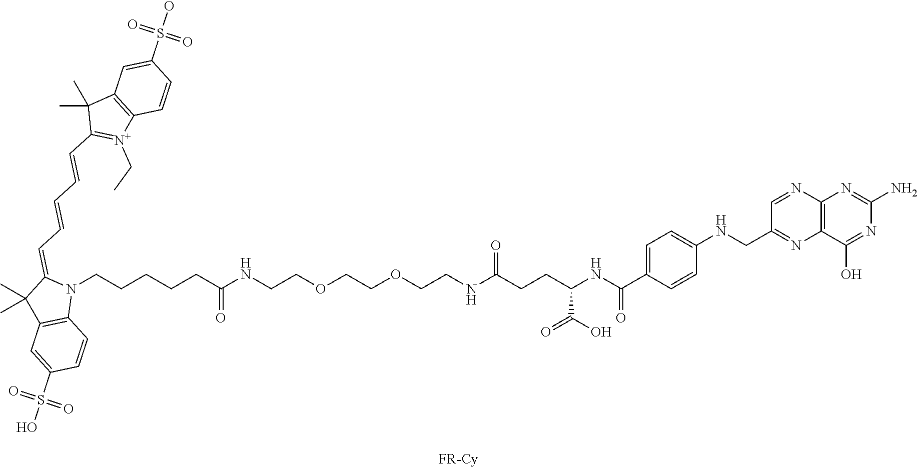 [SMILES: CC[N+]1=C(/C=C/C=C/C=C2\N(CCCCCC(=O)NCCOCCOCCNC(=O)CC[C@H](NC(=O)c3ccc(NCc4cnc5nc(N)nc(O)c5n4)cc3)C(=O)O)c3ccc(S(=O)(=O)O)cc3C2(C)C)C(C)(C)c2cc(S(=O)(=O)O)ccc21 ; "FR-Cy"]

Test compounds were resuspended in DMSO and 3-fold serial dilutions were made from 300-0.015 μM (100× final concentrations). Binding reactions were conducted in 200 μl final volume in 20 mM Hepes (pH 7.5) 250 mM NaCl 0.0150 Tween-20 1% DMSO with 2.5 nM folate receptor beta (Acro Biosystems) and 2 nM probe. Fluorescence polarization was measured using λex=620 nm, λem=688 nm on an Envision plate reader (Perkin Elmer) after 2 hr incubation time. Dose responses were conducted in duplicate and normalized to the response with DMSO (high) and 1 μM folic acid (low) on each plate. IC50 values were determined by fitting to 4-parameter curves in GraphPad Prism.

A summary of the binding and uptake results obtained for various example compounds is provided in Table 15.

TABLE 15
Summary of Example Compound Binding and Uptake

| | | TNF- | | | |
|---|---|---|---|---|---|
| Com- | | HTRF- | FOLR2- | New-uptake | New-uptake |
| pound | Example | biotin | FP | 100 nM-% | 10 nm-% |
| # | # | (nM) | (nM) | Example 46 | Example 46 |
| I-78 | Example 31 | 13 | 9.2 | 0.37 | 0.57 |
| I-79 | Example 32 | 17 | 3.2 | 1.51 | 1.71 |
| I-80 | Example 33 | 36 | 12 | 1.02 | 0.89 |
| I-81 | Example 34 | 39 | 7.1 | 0.32 | 0.19 |
| I-82 | Example 35 | 19 | 4.9 | 0.83 | 0.48 |
| I-83 | Example 36 | 28 | 5.3 | 1.04 | 0.88 |
| I-84 | Example 37 | 9 | 6.9 | 0.65 | 0.84 |
| I-85 | Example 38 | 13 | 12 | 0.32 | 0.55 |
| I-86 | Example 38 | 17 | 7.7 | 0.62 | 1.25 |
| I-87 | Example 38 | 22 | 8 | 1.57 | 2.50 |
| I-88 | Example 38 | 60 | 6.9 | 0.70 | 0.52 |
| I-89 | Example 38 | 42 | 8.4 | 0.96 | 1.44 |
| I-90 | Example 39 | 7.6 | 4.4 | 0.86 | 1.66 |
| I-91 | Example 40 | 17.5 | 138 | 0.25 | 0.40 |
| I-98 | Example 42 | 19 | 133 | 0.26 | 0.31 |
| I-92 | | 20 | 7.6 | 1.00 | 1.00 |
| I-93 | Example 43 | 18 | 6.6 | 1.09 | 1.65 |
| I-97 | | ND | 17 | 0.16 | 0.20 |
| I-94 | Example 43 | 22 | 9.3 | 0.81 | 0.83 |
| I-95 | Example 46 | 26 | 11 | 0.34 | 0.32 |
| I-96 | Example 51 | 56 | 20 | 0.39 | 0.34 |
| I-45 | Example 40 | 25 | 4.2 | 0.80 | 1.44 |

ND: No Data

### Folate Receptor SPR Assay

Folate receptor SPR binding assays were conducted on a Biacore 8K instrument (Cytiva) using biotinylated folate receptor beta (Acro Biosystems). Briefly, folate receptor was immobilized at -550 RU on a streptavidin chip (Cytiva) in 10 mM HEPES pH 7.5, 150 mM NaCl, 0.05% Tween 20. Multi-cycle kinetic studies were conducted with 8 compound concentrations using 2-fold dilutions in 10 mM HEPES pH 7.5, 150 mM NaCl, 0.05% Tween 20, 2% DMSO at 25° C. A flow rate or 75 μl/mmn with an association time of 120 s and a dissociation time of 600 s were used. Surfaces were regenerated with 4 M MgCl₂ at 30 μl/min for 30 s. Data were fit to a 1:1 model to determine the reported binding parameters.

A summary of the folate receptor SPR assay results obtained for various example compounds is provided in Table 16.

TABLE 16
Example compound FOLR2 binding

| | | | | | | Expected | |
|---|---|---|---|---|---|---|---|
| | | FOLR2Biotin - | ka | kd | Rmax | Rmax | Chi2 |
| Compound # | Example # | SPR Kd (M) | (1/Ms) | (1/s) | (RU) | (RU) | (RU2) |
| I-82 | Example 35 | 3.1E−11 | 1.91E+07 | 5.91E−04 | 14.5 | 23.2 | 1.90E−02 |
| I-84 | Example 37 | 5.9E−12 | 2.59E+07 | 1.53E−04 | 19.7 | 22.1 | 5.66E−03 |
| I-85 | Example 38 | 8.8E−11 | 1.27E+07 | 1.12E−03 | 11.9 | 23.5 | 4.88E−03 |
| I-86 | Example 38 | 2.3E−11 | 1.53E+07 | 3.44E−04 | 18.1 | 23.5 | 5.47E−02 |
| I-87 | Example 38 | 3.5E−11 | 1.97E+07 | 6.92E−04 | 18.7 | 23.3 | 3.16E−02 |
| I-89 | Example 38 | 2.23E−10 | 1.51E+06 | 3.37E−04 | 31.1 | 26.4 | 3.04E−02 |
| I-90 | Example 39 | 4.96E−12 | 2.62E+07 | 1.30E−04 | 13.3 | 21 | 3.66E−02 |
| I-91 | Example 40 | 2.01E−10 | 2.58E+06 | 5.17E−04 | 13.6 | 22.3 | 2.90E−02 |
| I-98 | Example 42 | 1.72E−10 | 4.49E+06 | 7.74E−04 | 14.8 | 22.9 | 2.05E−01 |
| I-92 | | 8.67E−12 | 1.00E+07 | 8.70E−05 | 17.1 | 21.4 | 1.83E−01 |
| I-93 | Example 43 | 1.09E−11 | 1.63E+07 | 1.78E−04 | 17.7 | 23.3 | 1.06E−01 |
| I-97 | | 9.14E−11 | 8.74E+06 | 7.98E−04 | 17.1 | 23.5 | 5.53E−02 |
| I-94 | Example 43 | 1.94E−11 | 8.72E+06 | 1.69E−04 | 15.4 | 23.3 | 9.32E−03 |
| I-95 | Example 46 | 2.15E−11 | 1.56E+07 | 3.37E−04 | 14.8 | 22 | 2.07E−01 |
| I-96 | Example 41 | 1.01E−10 | 1.27E+07 | 1.29E−03 | 17.1 | 22.7 | 3.26E−02 |

ND: No Data

### Ternary Complex SPR Assay

Folate receptor-TNFα ternary complex SPR assays were conducted on a Biacore 8K instrument (Cytiva) using biotinylated folate receptor beta (Acro Biosystems). Folate receptor was immobilized at -1800 RU on a streptavidin chip (Cytiva) in 10 mM HEPES pH 7.5, 150 mM NaCl, 0.05% Tween 20. Untagged TNF was preincubated with 10 nM compound for 6 hr in 10 mM HEPES pH 7.5, 150 mM NaCl, 0.05 Tween 20, 2% DMSO at 25° C. Samples were then injected over the folate receptor surface at 30 μl/min with an association time of 110 s and a dissociation time of 600 s. Surfaces were regenerated with 4 M MgCl₂ at 30 μl/mm for 30 s. The maximum measured response was normalized by comparison to the maximum expected response for each compound.

A summary of the ternary complex SPR assay results obtained for various example compounds is provided in Table 17.

TABLE 17
Example compound ternary complex formation
with TNFα and streptavidin immobilized FOL2R

| | | TC 60 nM | TC 150 nM- | TC 300 nM- |
|---|---|---|---|---|
| | | taggless TNF | tagless TNF | tagless TNF |
| Compound | Example | normalized | Normalized | Normalized |
| # | # | (%) | (%) | (%) |
| I-81 | Example 34 | 4 | ND | 12.1 |
| I-82 | Example 35 | 15.7 | ND | 24.9 |
| I-83 | Example 36 | 14.3 | ND | 17.5 |
| I-84 | Example 37 | 10.8 | 17.4 | 17.3 |
| I-85 | Example 38 | 9.7 | ND | 15.1 |
| I-86 | Example 38 | ND | 20.1 | 22.8 |
| I-87 | Example 38 | ND | 25.7 | 32.8 |
| I-89 | Example 38 | 11.6 | ND | 15 |
| I-90 | Example 39 | ND | 20.4 | 20.6 |
| I-91 | Example 40 | ND | 8.8 | 9.3 |
| I-98 | Example 42 | ND | 16.7 | 19.4 |
| I-92 | | 15.3 | 16.9 | 18.7 |
| I-93 | Example 43 | ND | 16.1 | 18.2 |
| I-97 | | ND | 18.4 | 21.1 |
| I-94 | Example 43 | ND | 16.3 | 18.5 |
| I-95 | Example 46 | ND | 17.3 | 18.6 |
| I-96 | Example 41 | 12.3 | 17.1 | 19.2 |
| I-45 | Example 40 | ND | 16.3 | 17.6 |

ND: No Data

### 6.3.14. Example 71: Pharmacokinetic Study in Mice with OMA-Folate Compound Vs OMA-Attenuated Folate Compound

Pharmacokinetic studies were undertaken to determine the pharmacokinetic profile of an OMA linked example folate compound (compound I-4B) vs an OMA linked attenuated folate compound having reduced affinity for the folate receptor (also referred to herein as “OMA-reference” or “OMA-ref.”).

The attenuated folate compound (reference compound) has the following structure:

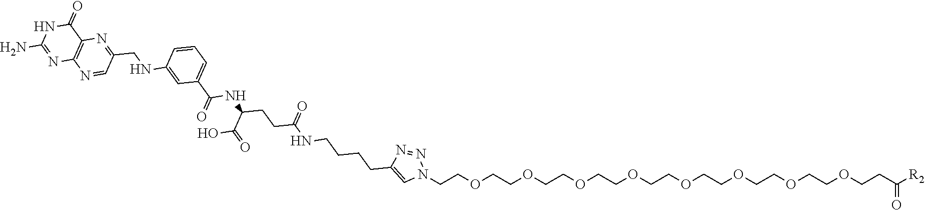

where R² is pentafluorophenyl ester (OPFP)

Protocol: Female C57BL/6 mice were dosed via IV with 5 mg/kg of OMA-I-4B, and OMA-reference. Serum samples were collected at 0.083, 0.25, 0.5, 1, 2, 4, 8, 24, 48, 72, and 168 hours after dosing with 3 animals per timepoint.

Serum hIgG1 concentrations were measured by ELISA.

FIG. 11 illustrates affinity-dependent clearance of an OMA-folate example compound (OMA-I-4B) as compared to OMA by itself and an OMA linked attenuated folate compound having reduced affinity for the folate receptor (OMA-ref).

These results demonstrate that increased clearance is observed with higher affinity binding to FR.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A cell surface folate receptor binding compound of formula (I): 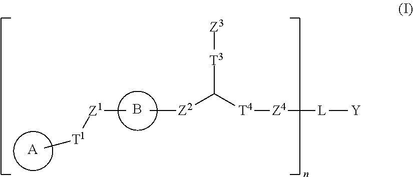
or a salt thereof,
wherein:
T¹ is an optionally substituted (C₁-C₃)alkylene;
Z¹ is selected from —NR²³—, —O—, —S—, and optionally substituted (C₁-C₃)alkylene, where R²³ is H, optionally substituted (C₁-C₆)alkyl, or R²³ forms a 5 or 6 membered cycle together with an atom of the B-ring;
B is a ring system selected from optionally substituted aryl, optionally substituted heteroaryl, optionally substituted heterocycle, optionally substituted cycloalkyl, and optionally substituted bridged bicycle;
Z² is absent, or a linking moiety selected from optionally substituted amide, optionally substituted sulfonamide, optionally substituted urea, optionally substituted thiourea, —NR²¹—, —O—, —S—, and optionally substituted (C₁-C₆)alkylene;
Z³ is absent, carboxyl, or carboxyl bioisostere, or a prodrug thereof;
T³ is absent, or is selected from optionally substituted (C₁-C₆)alkylene;
T⁴ is optionally substituted (C₁-C₆)alkylene (e.g., —CH₂CH₂—), or is absent;
Z⁴ is a linking moiety;
each R²¹ is independently selected from H, and optionally substituted (C₁-C₆)alkyl;
n is 1 to 100;
L is a linker;
Y is a moiety of interest; and
A is a ring system of formula (II): 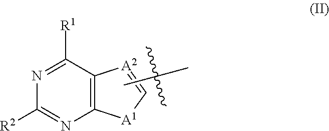
or a tautomer thereof, wherein:
R¹ and R² are independently selected from H, OH, NR²¹, and optionally substituted (C₁-C₆)alkyl;
A¹ is selected from —N═CR³—, —CR³═N—, —CR³═CR³—, NR²¹, S, O, and C(R⁴)₂;
A² is selected from N, and CR³;
each R³ is independently selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each R⁴ is independently selected from H, halogen, and optionally substituted (C₁-C₆)alkyl;
with the proviso that at least one of following applies:
6) T³ is optionally substituted (C₁-C₆)alkylene;
7) L is a non-cleavable linker and Y is an extracellular target-binding moiety;
8) when A is of formula (II-A) or (II-A′), or a tautomer thereof: 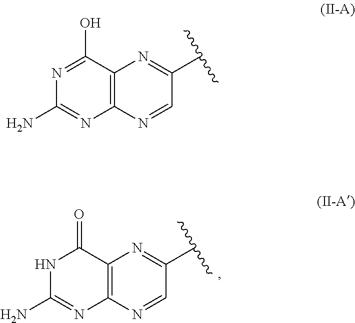 [SMILES: *c1cnc2nc(N)nc(O)c2n1 ; *c1cnc2nc(N)[nH]c(=O)c2n1]
then Z¹ is not NR²¹, and/or B is not 1,4-linked phenyl;
9) when A is of formula (II-B), or a tautomer thereof: 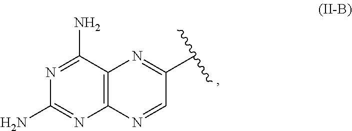 [SMILES: *c1cnc2nc(N)nc(N)c2n1]
then Z¹ is not NR²¹, and/or B is not 1,4-linked phenyl; and/or
10) when A is of formula (II-C) or (II-C′), or a tautomer thereof: 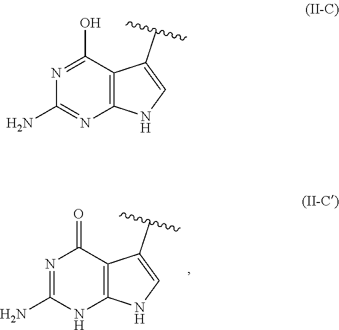 [SMILES: *c1c[nH]c2nc(N)nc(O)c12 ; *c1c[nH]c2[nH]c(N)nc(=O)c12]
then T¹-Z¹ is not —CH₂CH₂—, and/or B is not phenyl.

2. The compound of claim 1, wherein T³ is optionally substituted (C₁-C₆)alkylene.

3. The compound of claim 2, wherein T³ is (C₁-C₃)alkylene.

4. The compound of claim 3, wherein T³ is —CH₂CH₂—.

5. The compound of any one of claims 1 to 4, wherein T⁴ is absent.

6. The compound of claim 5, wherein the compound is of formula (IIIA): 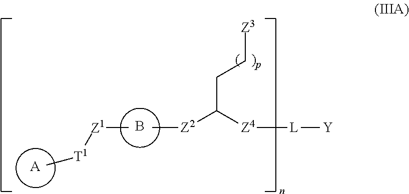
wherein p is 0 or 1.

7. The compound of claim 1, wherein T³ is absent.

8. The compound of claim 7, wherein T⁴ is optionally substituted (C₁-C₆)alkylene.

9. The compound of claim 8, wherein T⁴ is (C₁-C₃)alkylene.

10. The compound of claim 9, wherein T⁴ is —CH₂CH₂—.

11. The compound of any one of claims 7 to 10, wherein the compound is of formula (IIIB): 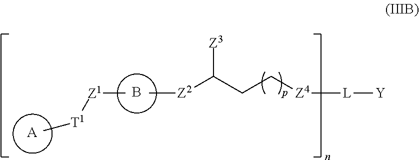
wherein p is 0 or 1.

12. The compound of any one of claims 1 to 11, wherein Z³ is selected from —COOH, —COOR²², —CH₂OH, —CH₂OR²², —CN, and tetrazole, wherein R²² is optionally substituted (C₁-C₆)alkyl.

13. The compound of claim 12, wherein Z³ is selected from: 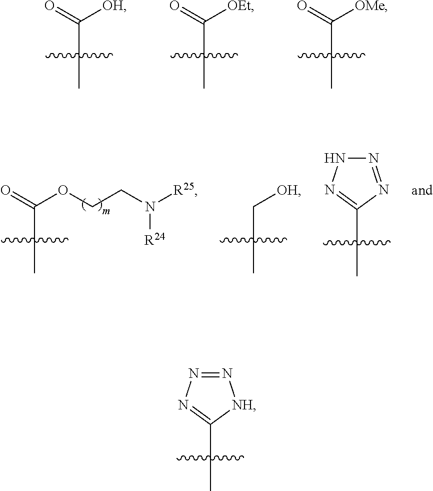
wherein:
R²⁴ and R²⁵ are independently selected from H and optionally substituted (C₁-C₆)alkyl, or R²⁴ and R²⁵ are cyclically linked to provide an optionally substituted 5 or 6-membered heterocycle; and
m is 1 to 5.

14. The compound of claim 13, wherein Z³ is COOH.

15. The compound of claim 13, wherein Z³ is 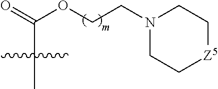
wherein
Z⁵ is O, NH or NR²¹; and
R²¹ is (C₁-C₆)alkyl.

16. The compound of claim 15, wherein Z⁵ is O, NH or NMe, and m is 1.

17. The compound of any one of claims 1 to 10, wherein Z³ is absent, and the compound is of formula (IIIC): 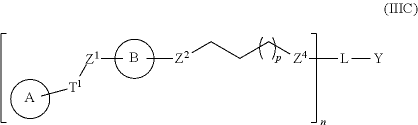
wherein p is 0 or 1.

18. The compound of claim 10, wherein Z² is —CONR²¹—, wherein R²¹ is selected from H, and optionally substituted (C₁-C₆)alkyl.

19. The compound of any one of claims 12 to 18, wherein Z² is —CONR²¹—, —NR²¹CO—, —SO₂NR²¹—, —NR²¹C(═O)NR²¹—, or —NR²¹C(═S)NR²¹, wherein each R²¹ is independently selected from H, and optionally substituted (C₁-C₆)alkyl.

20. The compound of any one of claims 12 to 19, wherein Z⁴ is a linking moiety selected from —CONR²¹—, —NR²¹—, —O—, —S—, optionally substituted aryl optionally substituted heterocycle, optionally substituted spiroheterocycle, and optionally substituted heteroaryl, wherein R²¹ is selected from H, and optionally substituted (C₁-C₆)alkyl.

21. The compound of claim 20, wherein Z⁴ is a linking group selected from:

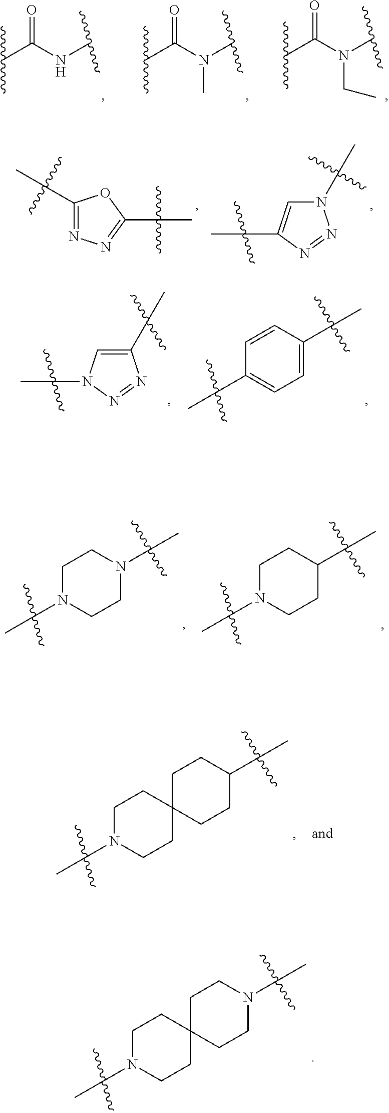

22. The compound of any one of claims 12 to 21, wherein —Z²CH(-T³-Z³)T⁴Z⁴—of formula (I) is selected from the following structures: 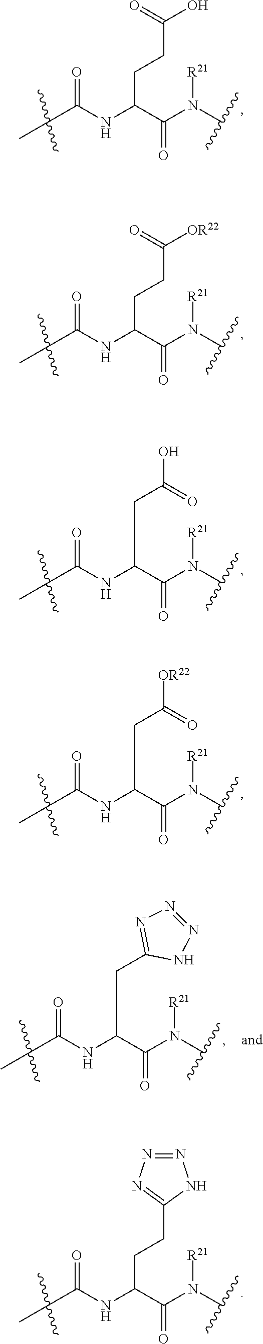
or a tautomer thereof, or a salt thereof.

23. The compound of any one of claims 12 to 21, wherein —Z²CH(-T³-Z³)T⁴Z⁴—of formula (I) is selected from the following structures: 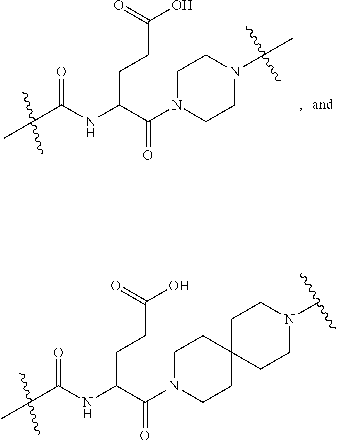 [SMILES: *C(=O)NC(CCC(=O)O)C(=O)N1CCN(*)CC1 ; *C(=O)NC(CCC(=O)O)C(=O)N1CCC2(CCN(*)CC2)CC1]
or a tautomer thereof, or a salt thereof.

24. The compound of any one of claims 12 to 21, wherein —Z²CH(-T³-Z³)T⁴Z⁴—of formula (I) is selected from the following structures: 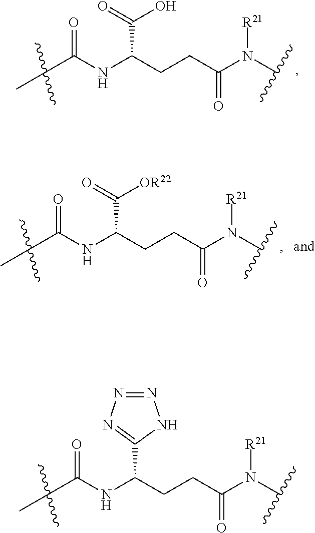
or a tautomer thereof, or a salt thereof.

25. The compound of any one of claims 12 to 21, wherein —Z²CH(-T³-Z³)T⁴Z⁴—of formula (I) is selected from the following structures: 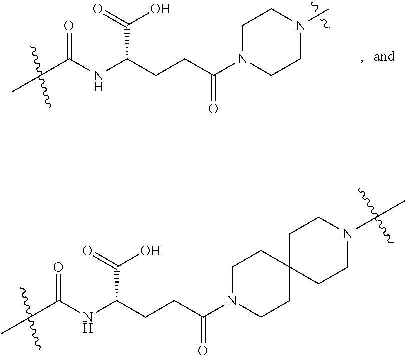
or a tautomer thereof, or a salt thereof.

26. The compound of any one of claims 12 to 21, wherein —Z²CH(-T³-Z³)T⁴Z⁴—of formula (I) is selected from the following structures: 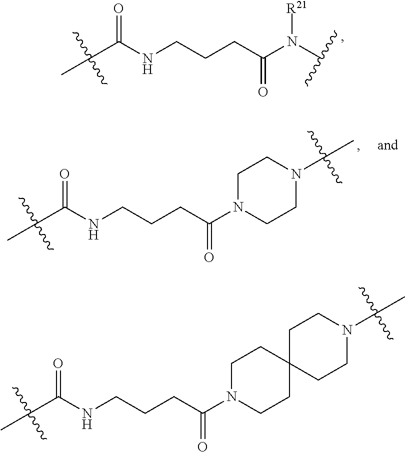
or a tautomer thereof, or a salt thereof.

27. The compound of any one of claims 1 to 26, wherein A¹ of ring system A is independently —N═CR³—, —CR³═N—, or —CR³═CR³—.

28. The compound of claim 27, wherein A is of formula (IIA): 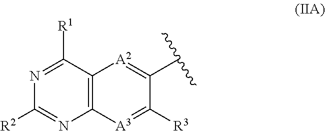
or a tautomer thereof, or a salt thereof, wherein:
A² is selected from N, and CR³;
A³ is independently selected from N, and CR²¹.

29. The compound of claim 28, wherein A² and A³ are each N.

30. The compound of claim 28, wherein A² and A³ are each independently CR³.

31. The compound of any one of claims 27 to 30, wherein each R³ is H.

32. The compound of any one of claims 27 to 31, wherein R² is —NH₂.

33. The compound of any one of claims 27 to 31, wherein R² is optionally substituted (C₁-C₆)alkyl.

34. The compound of any one of claims 27 to 31, wherein R² is —CH₃ or —CH₂OH.

35. The compound of any one of claims 27 to 34, wherein R¹ is OH.

36. The compound of any one of claims 27 to 35, wherein A is selected from: 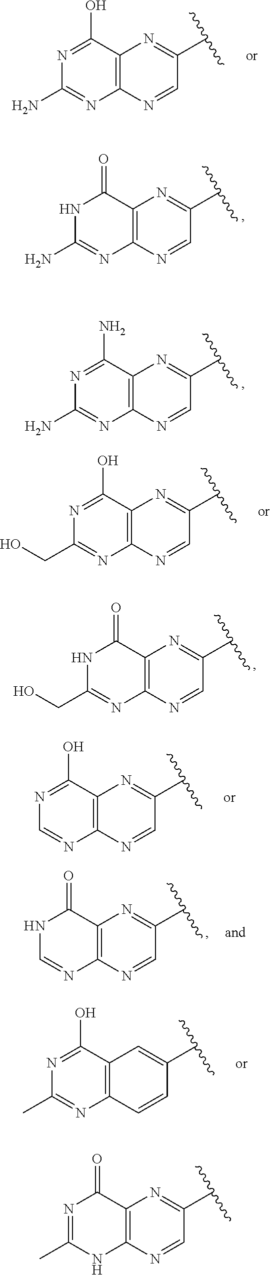
or a tautomer thereof.

37. The compound of any one of claims 1 to 26, wherein A¹ of ring system A is NR²¹, S, O, or C(R²¹)₂.

38. The compound of claim 37, wherein A is of formula (IIB) or (IIC): 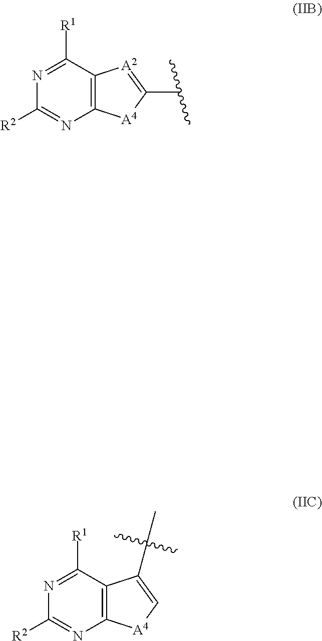
or a tautomer thereof, or a salt thereof, wherein A⁴ is selected from NR²¹, S, and O.

39. The compound of claim 38, wherein A⁴ is NR²¹.

40. The compound of claim 38 or 39, wherein A² is CR³.

41. The compound of any one of claims 37 to 40, wherein R² is —NH₂.

42. The compound of any one of claims 37 to 40, wherein R² is optionally substituted (C₁-C₆)alkyl.

43. The compound of any one of claims 37 to 42, wherein R¹ is OH.

44. The compound of any one of claims 37 to 43, wherein A is: 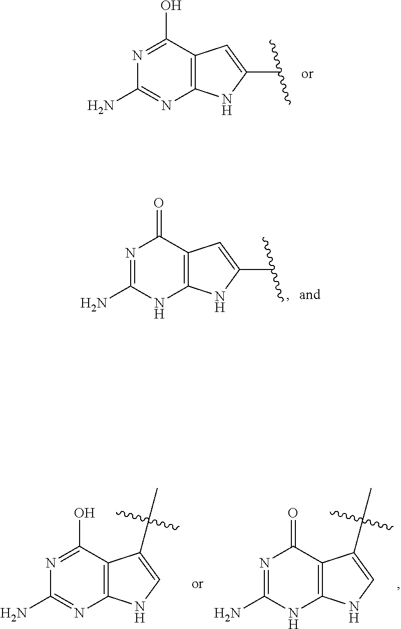 [SMILES: *c1cc2c(O)nc(N)nc2[nH]1 ; *c1cc2c(=O)nc(N)[nH]c2[nH]1 ; *c1c[nH]c2nc(N)nc(O)c12 ; *c1c[nH]c2[nH]c(N)nc(=O)c12]
or a tautomer thereof.

45. The compound of any one of claims 27 to 44, wherein T¹ is CH₂ or CH₂CH₂.

46. The compound of any one of claims 27 to 45, wherein Z¹ is NR.

47. The compound of claim 46, wherein R²¹ is H.

48. The compound of claim 46, wherein R²¹ is methyl, ethyl, propyl, or propargyl.

49. The compound of any one of claims 27 to 45, wherein Z¹ is O or S.

50. The compound of any one of claims 27 to 45, wherein T¹-Z¹ is optionally substituted (C₁-C₆)alkylene.

51. The compound of claim 50, wherein T¹-Z¹ is —CH₂CH₂—.

52. The compound of claim 50, wherein T¹-Z¹ is —CH₂CH₂CH₂CH₂—or —CH₂CH₂CH₂—.

53. The compound of any one of claims 1 to 52, wherein B is selected from optionally substituted phenyl, optionally substituted pyridyl, optionally substituted pyrimidine, optionally substituted thiophene, optionally substituted pyrrole, optionally substituted furan, optionally substituted oxazole, optionally substituted thiazole, optionally substituted cyclohexyl, optionally substituted cyclopentyl, optionally substituted indole, and optionally substituted bicycloalkyl.

54. The compound of claim 53, wherein B is selected from optionally substituted 1,4-phenylene, optionally substituted 1,3-phenylene, optionally substituted 2,5-pyridylene, optionally substituted 2,5-thiophene, optionally substituted 1,4-cyclohexyl, and optionally substituted 1,3-bicyclo[1.1.1]pentane.

55. The compound of claim 53 or 54, wherein —B—Z²—is selected from: 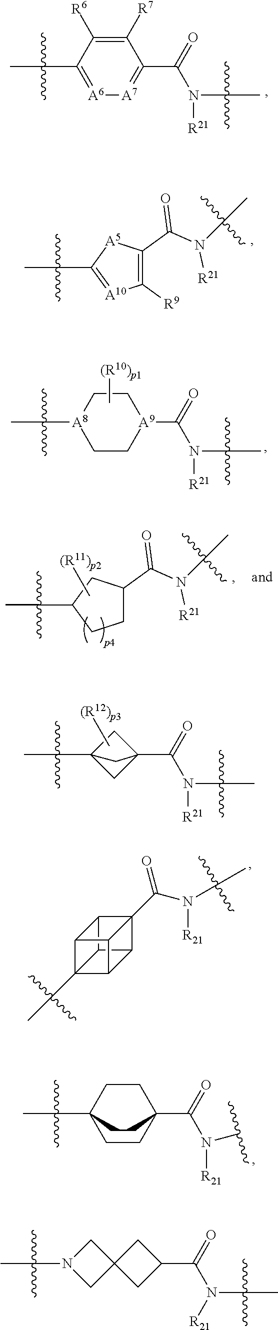
wherein:
A⁵ is selected from NR²¹, S, O, C(R⁵)₂;
A⁶-A⁹ are independently selected from N, and CR; R²¹ is selected from H, and optionally substituted (C₁-C₆)alkyl;
A¹⁰ is selected from N, and CR;
each R⁵ to R¹² is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
p1 is 0 to 10;
p2 is 0 to 14;
p3 is 0 to 4; and
p4 0 to 4.

56. The compound of claim 55, wherein B—Z² is: 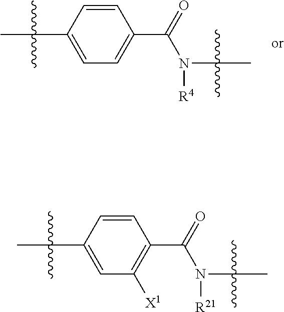
wherein X¹ is halogen.

57. The compound of claim 50, wherein A-T¹-Z¹—B—is selected from one of the following: 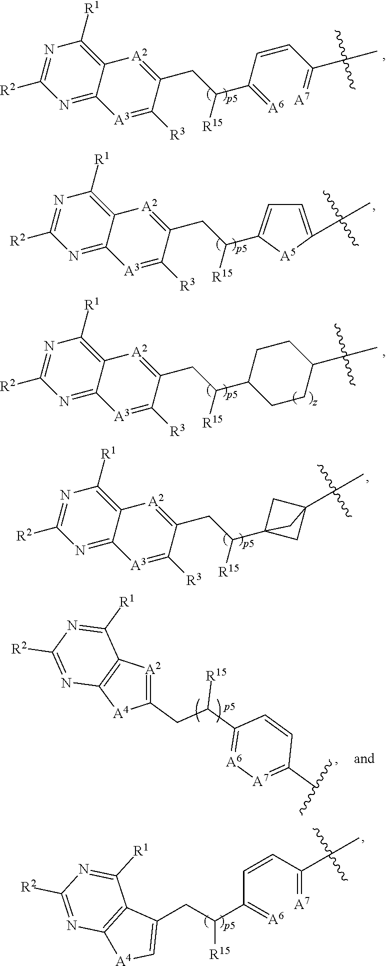
wherein:
A⁵ is selected from NR²¹, S, O, C(R⁵)₂;
A⁶ and A⁷ are independently selected from N, and, CR⁵;
z is 0 to 3
each R⁵ and R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each p5 is independently 1 to 3.

58. The compound of claim 1, wherein the compound comprises a cell surface folate receptor ligand selected from: 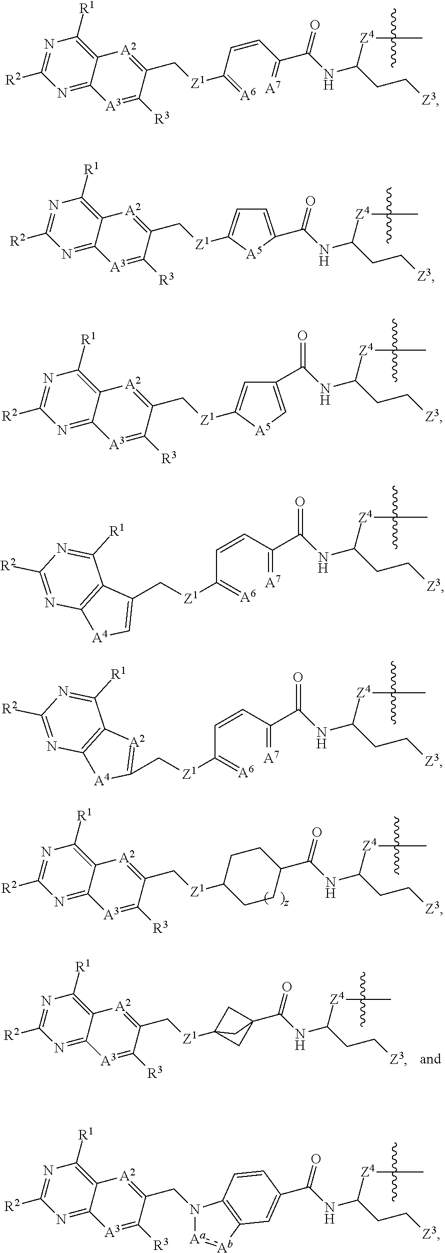
wherein:
A⁵ is selected from NR²¹, S, O, C(R⁵)₂;
A⁶ and A⁷ are independently selected from N, and, CR⁵;
z is 0 to 3; [symbol]
is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

59. The compound of claim 1, wherein the compound comprises a cell surface folate receptor ligand selected from: 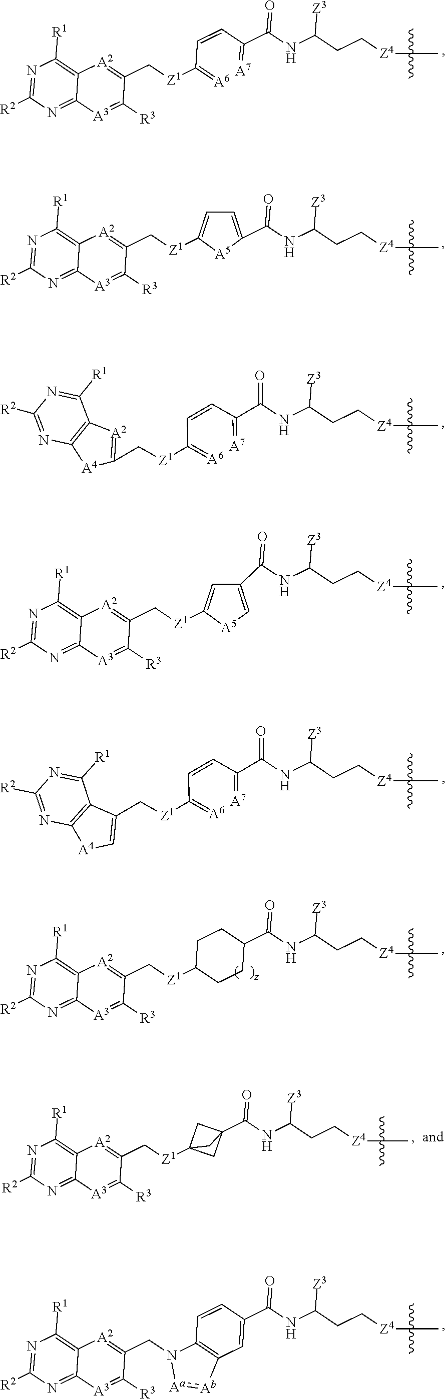
wherein:
A⁵ is selected from NR²¹, S, O, C(R⁵)₂;
A⁶ and A⁷ are each independently selected from N, and, CR⁵;
z is 0 to 3; [symbol]
is a single bond or a double bond;
wherein when [symbol] is a single bond A^{a} is selected from C(R⁵)₂, and C═O, and A^{b} is selected from C(R⁵)₂, and NR²¹; and
when [symbol] is a double bond A^{a} is CR⁵, and A^{b} is selected from CR⁵ and N; and
wherein each R is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵; —CONHR²⁵, and —NHCOR²⁵.

60. The compound of any one of claims 1 to 59, wherein n is 1.

61. The compound of any one of claims 1 to 59, wherein n is at least 2.

62. The compound of claim 61, wherein n is 2 to 20.

63. The compound of any one of claims 1 to 62, wherein L comprises a backbone of at least 10 consecutive atoms.

64. The compound of any one of claims 61 to 63, wherein L comprises one or more linking moieties independently selected from —C₁₋₆-alkylene-, —NHCO—C₁₋₆-alkylene-, —CONH—C₁₋₆-alkylene-, —NH C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, —NH—, and —NMe—, wherein p is 1 to 10.

65. The compound of claim 63, wherein L comprises repeating ethylene glycol moieties.

66. The compound of claim 63 or 64, wherein L comprises 1 to 20 ethylene glycol moieties.

67. The compound of any one of claims 1 to 66, wherein L is of formula (IV): 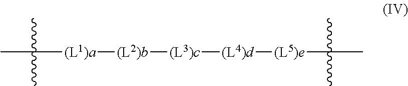
wherein
each L¹ to L⁵ is independently a linking moiety which together provide a linear or branched linker between Z⁴ and Y;
a is 1 or 2; and
b, c, d, and e are each independently 0, 1, or 2.

68. The compound of claim 67, wherein -(L¹)ₐ- comprises an optionally substituted alkyl or ethylene glycol linking moiety.

69. The compound of claim 67 or 68, wherein each L¹ is independently selected from:
—C₁₋₆-alkylene-, —(CH₂CH₂O)ₜ—, —C₁₋₆-alkylene-NR⁴CO—, —C₁₋₆-alkyleneCONH—, or OCH₂, wherein t is 1 to 20; and
R⁴ is independently selected from H, and optionally substituted (C₁-C₆)alkyl.

70. The compound of any one of claims 67 to 69, wherein:
each L² is independently selected from —NR⁴CO—C₁₋₆-alkylene-, —CONR⁴—C₁₋₆-alkylene, 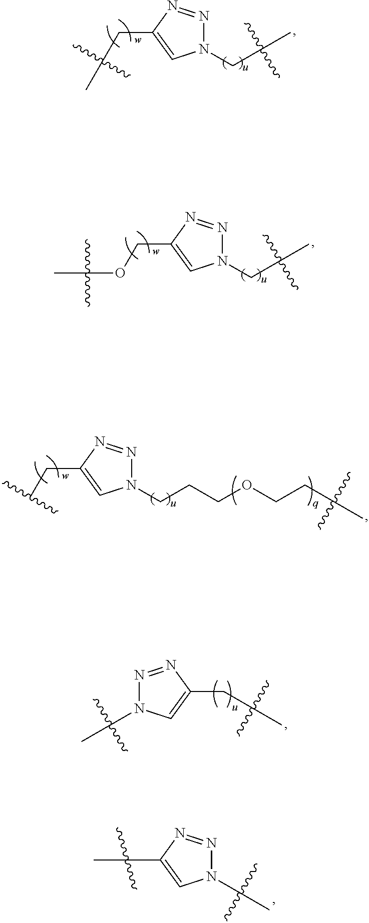
—OCH₂—, and —(OCH₂CH₂)_{q}—, wherein q is 1 to 10, u is 0 to 10, w is 1 to 10, and
R⁴ is independently selected from H, and optionally substituted (C₁-C₆)alkyl; and
each L⁴ is absent or independently selected from —C₁₋₆-alkylene-, —(CH₂CH₂O)ₜ—, —C₁₋₆-alkylene-NHCO—, —C₁₋₆-alkyleneCONH—, or OCH₂,
wherein t is 1 to 20.

71. The compound of any one of claims 67 to 70, wherein when n is 2 or more, at least one L³ is present and is a branched linking moiety.

72. The compound of any one of claims 67 to 71, wherein each L³ is independently selected from:
—OCH₂CH₂—, 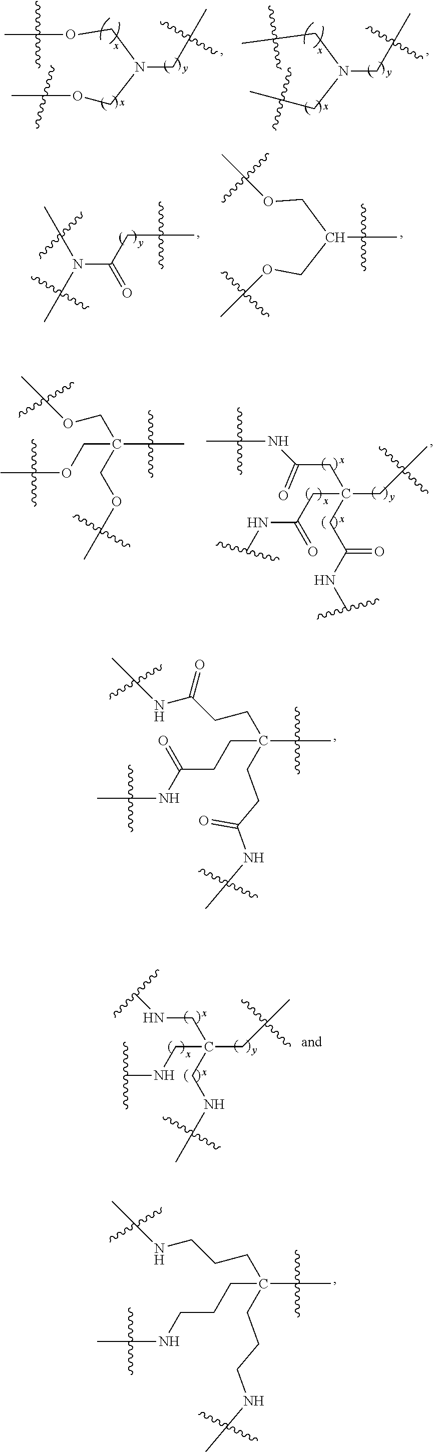
wherein each x and y are each independently 1 to 10.

73. The compound of any one of claims 67 to 72, wherein:
each L⁵ is independently —CH₂O—; —(CH₂CH₂O)ₜ—, —NR⁴CO—, —C₁₋₆-alkylene-, 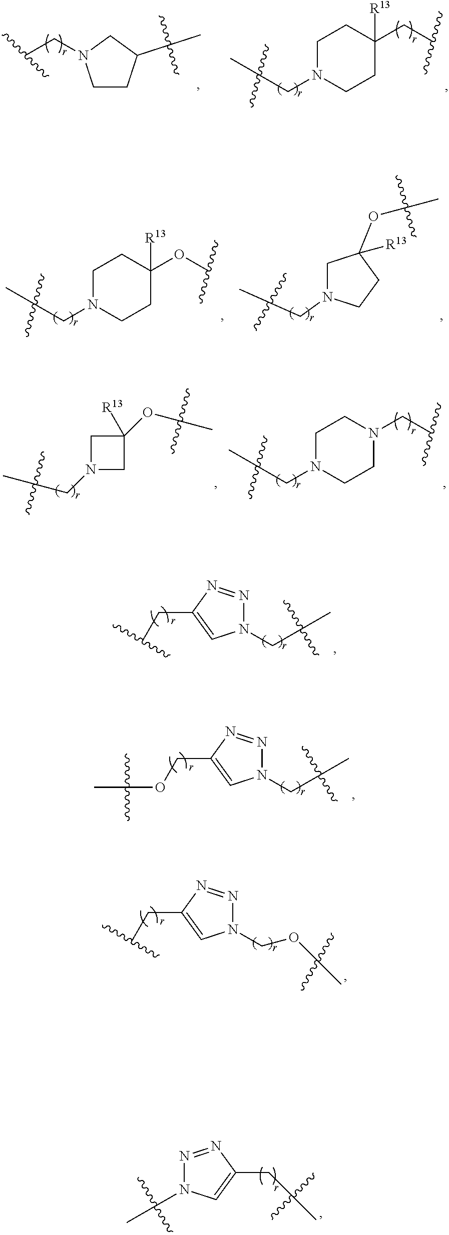
wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; and
each r independently 0 to 20.

74. The compound of any one of claims 67 to 73, wherein a is 1.

75. The compound of any one of claims 67 to 74, wherein at least one of b, c, d, and e is not 0.

76. The compound of any one of claims 67 to 75, wherein b, d, and e are each independently 1 or 2.

77. The compound of any one of claims 67 to 76, wherein a, b, d, and e are each 1, and c is 0.

78. The compound of any one of claims 67 to 77, wherein the linker L is selected from any one of the structures of Table 3.

79. The compound of any one of claims 1 to 78, wherein the compound comprises a cell surface folate receptor ligand of one of the structures of Tables 1 or 2.

80. The compound of any one of claims 1 to 79, wherein Y is selected from small molecule, dye, fluorophore, monosaccharide, polysaccharide, lipid, enzyme, enzyme substrate and chemoselective ligation group or precursor thereof.

81. The compound of any one of claims 1 to 80, wherein Y is a moiety that specifically binds an extracellular target protein.

82. The compound of claim 81, wherein the target protein is a membrane bound protein.

83. The compound of claim 81, wherein the target protein is a soluble extracellular protein.

84. The compound of any one of claims 1 to 83, wherein Y is a target-binding small molecule.

85. The compound of claim 84, wherein Y is a small molecule inhibitor or ligand of the target protein.

86. The compound of claim 85, wherein Y is an allosteric desymmetrization TNFα inhibitor.

87. The compound of any one of claims 1 to 85, wherein Y is a target-binding biomolecule.

88. The compound of claim 87, wherein the biomolecule is selected from peptide, protein, glycoprotein, polynucleotide, aptamer, and antibody or antibody fragment.

89. The compound of claim 88, wherein Y is selected from antibody, antibody fragment, chimeric fusion protein, an engineered protein domain, and D-protein binder of target protein.

90. The compound of claim 89, wherein Y is antibody or antibody fragment that specifically binds the target protein and the compound is of formula (VIIIa): 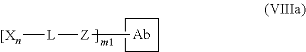
or a pharmaceutically acceptable salt thereof, wherein:
n is 1 to 20;
m1 is an average loading of 1 to 80;
each X is a moiety that binds to a cell surface folate receptor;
each L is a linker;
each Z is a residual moiety resulting from the covalent linkage of a chemoselective ligation group to a compatible group of Ab; and
A^{b} is the antibody or antibody fragment that specifically binds the target protein.

91. The compound of claim 90, wherein X is not folic acid, methotrexate, or pemetrexed.

92. The compound of claim 90 or 91, wherein X is selected from: 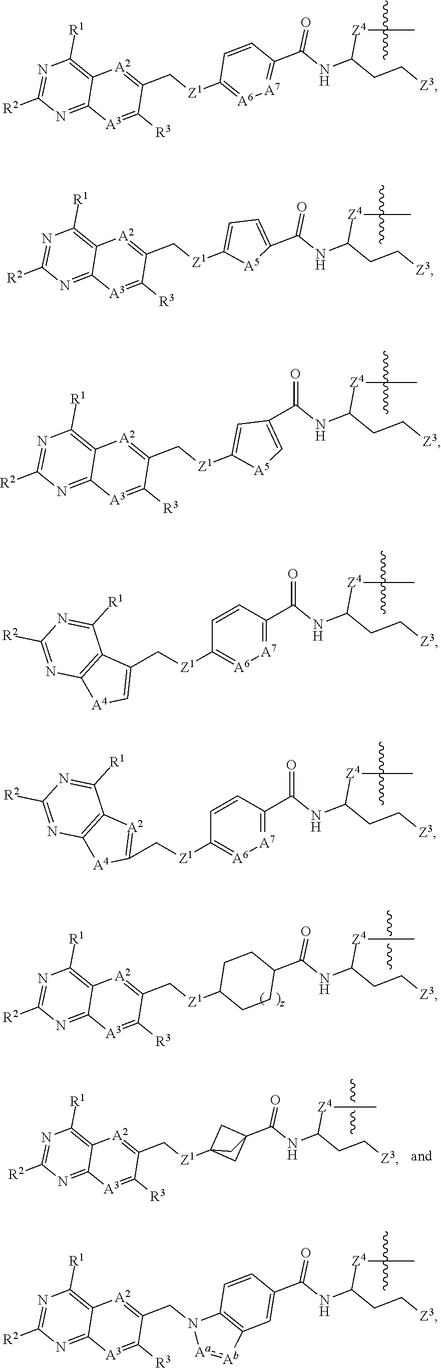
wherein:
A⁵ is selected from NR²¹, S, O, C(R⁵)₂;
A⁶ and A⁷ are independently selected from N, and, CR⁵;
z is 0 to 3; [symbol]
is a single bond or a double bond;
wherein when [symbol] is a single bond A^{a} is selected from C(R⁵)₂, and C═O, and A^{b} is selected from C(R⁵)₂, and NR²¹;
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵.

93. The compound of claim 90 or 91, wherein X is selected from: 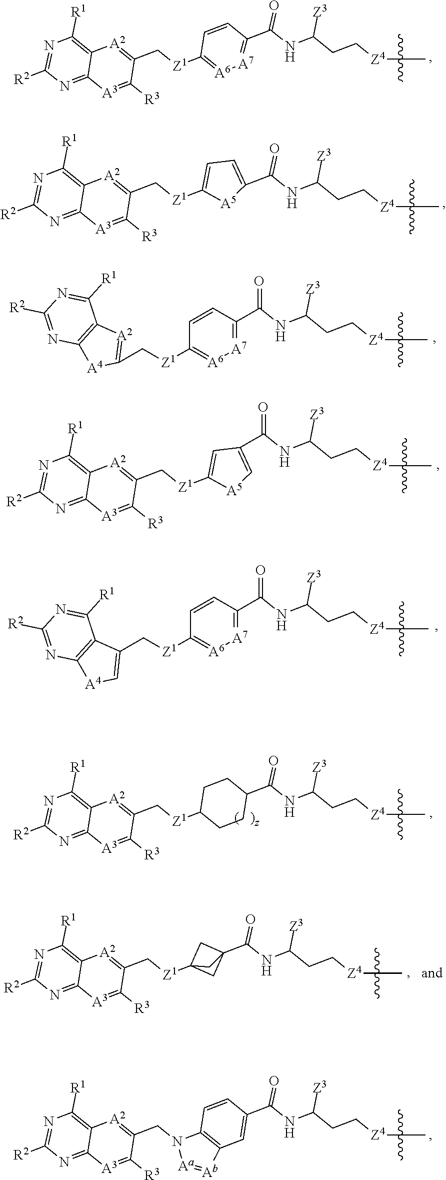
wherein:
A⁵ is selected from NR²¹, S, O, C(R⁵)₂;
A⁶ and A⁷ are each independently selected from N, and, CR⁵;
z is 0 to 3; [symbol]
is a single bond or a double bond;
wherein when [symbol] is a single bond A^{a} is selected from C(R⁵)₂, and C═O, and A^{b} is selected from C(R⁵)₂, and NR²¹;
when [symbol] is a double bond A^{a} is CR⁵, and A^{b} is selected from CR⁵ and N; and
wherein each R⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵, The compound of any one of claims 85 to 88, wherein n is 1 to 6.

94. The compound of claim 93, wherein n is 2 or less.

95. The compound of claim 94, wherein n is 1.

96. The compound of any one of claims 91 to 94, wherein n is at least 2.

97. The compound of claim 96, wherein n is 2.

98. The compound of claim 96, wherein n is 3.

99. The compound of claim 96, wherein n is 4.

100. The compound of any one of claims 91 to 99, wherein m1 is 1 to 20.

101. The compound of claim 100, wherein m1 is 1 to 12.

102. The compound of claim 100 or 101, wherein m1 is 2 or more.

103. The compound of claim 100 or 101, wherein m1 is at 3 or more.

104. The compound of claim 100 or 101, wherein m1 is 4 or more.

105. The compound of any one of claims 91 to 104, wherein Z is a residual moiety resulting from the covalent linkage of a thiol-reactive chemoselective ligation group to one or more cysteine residue(s) of Ab.

106. The compound of any one of claims 91 to 104, wherein Z is a residual moiety resulting from the covalent linkage of an amine-reactive chemoselective ligation group to one or more lysine residue(s) of Ab.

107. The compound of any one of claims 91 to 106, wherein the antibody or antibody fragment is an IgG antibody.

108. The compound of any one of claims 91 to 106, wherein the antibody or antibody fragment is a humanized antibody.

109. The compound of any one of claims 91 to 108, wherein the antibody or antibody fragment specifically binds to a secreted or soluble protein.

110. The compound of any one of claims 91 to 108, wherein the antibody or antibody fragment specifically binds to a cell surface receptor.

111. A method of internalizing a target protein in a cell comprising a cell surface folate receptor, the method comprising:
contacting a cellular sample comprising the cell and the target protein with an effective amount of a compound according to any one of claims 1 to 110, wherein the compound specifically binds the target protein and specifically binds the cell surface folate receptor to facilitate cellular uptake of the target protein.

112. The method of claim 111, wherein the target protein is a membrane bound protein.

113. The method of claim 111, wherein the target protein is an extracellular protein.

114. The method of any one of claims 111 to 113, wherein the compound or conjugate comprises an antibody or antibody fragment (Ab) that specifically binds the target protein.

115. A method of reducing levels of a target protein in a biological system, the method comprising:
contacting the biological system with an effective amount of a compound according to any one of claims 1 to 110, wherein the compound specifically binds the target protein and specifically binds a cell surface receptor of cells in the biological system to facilitate cellular uptake and degradation of the target protein.

116. The method of claim 115, wherein the biological system comprises cells that comprise a folate cell surface receptor.

117. The method of claim 115 or 116, wherein the biological system is a human subject.

118. The method of any one of claims 115 to 117, wherein the biological system is an in vitro cellular sample.

119. The method of any one of claims 115 to 118, wherein the target protein is a membrane bound protein.

120. The method of any one of claims 115 to 118, wherein the target protein is an extracellular protein.

121. A method of treating a disease or disorder associated with a target protein, the method comprising:
administering to a subject in need thereof an effective amount of a compound according to any one of claims 1 to 110, wherein the compound specifically binds the target protein.

122. The method of claim 121, wherein the disease or disorder is an inflammatory disease.

123. The method of claim 121, wherein the disease or disorder is an autoimmune disease.

124. The method of claim 121, wherein the disease or disorder is a cancer.
